(19) United States
(12) Patent Application Publication
(10) Pub. No.: US 2025/0250584 A1
(43) Pub. Date: Aug. 7, 2025

(54) METHODS AND COMPOSITIONS FOR GENOMIC INTEGRATION

(71) Applicant: Myeloid Therapeutics, Inc., Cambridge, MA (US)

(72) Inventors: Daniel Getts, Medfield, MA (US); Yuxiao Wang, Belmont, MA (US); Namita Bisaria, Somerville, MA (US); Inna Shcherbakova, Holliston, MA (US); Socheata Ly, North Billerica, MA (US)

(21) Appl. No.: 19/187,426

(22) Filed: Apr. 23, 2025

Related U.S. Application Data
(63) Continuation of application No. 18/157,052, filed on Jan. 19, 2023, now Pat. No. 12,319,925.
Application No. 18/157,052 is a continuation of application No. PCT/US22/28831, filed on May 11, 2022.
(60) Provisional application No. 63/187,117, filed on May 11, 2021.
(60) Provisional application No. 63/254,791, filed on Oct. 12, 2021.
(60) Provisional application No. 63/274,907, filed on Nov. 2, 2021.

(51) Int. Cl. C12N 15/85; A61K 31/713; C12N 15/90

(52) U.S. Cl. CPC C12N 15/85; A61K 31/713; C12N 15/90; C07K 2319/03; C07K 2319/09; C07K 2319/30; C07K 2319/33; C12N 2310/20; C12N 2800/80; C12N 2800/90; C12N 2830/50; C12N 2840/203

(57) ABSTRACT

Methods and composition for modulating a target genome and stable integration of a transgene of interest into the genome of a cell are disclosed.

### CROSS REFERENCE

This application is a continuation application of U.S. application Ser. No. 18/157,052, filed on Jan. 19, 2023, which is a continuation application of the international application, PCT/US22/28831, filed on May 11, 2022; which claims the benefit of priority to U.S. Provisional Application No. 63/187,117, filed on May 11, 2021, U.S. Provisional Application No. 63/254,791, filed on Oct. 12, 2021, and U.S. Provisional Application No. 63/274,907, filed on Nov. 2, 2021, each of which is hereby incorporated by reference in its entirety.

### SEQUENCE LISTING

The instant application contains a Sequence Listing which has been filed electronically in XML format and is hereby incorporated by reference in its entirety. Said XML copy, created Jan. 27, 2023 is named 56371-723-301_SL_1-19-23_revl-27-23.xml and is 595,456 bytes in size.

### BACKGROUND

Cell therapy is a rapidly developing field for addressing difficult to treat diseases, such as cancer, persistent infections and certain diseases that are refractory to other forms of treatment. Cell therapy often utilizes cells that are engineered ex vivo and administered to an organism to correct deficiencies within the body. An effective and reliable system for manipulation of a cell's genome is crucial, in the sense that when the engineered cell is administered into an organism, it functions optimally and with prolonged efficacy. Likewise, reliable mechanisms of genetic manipulation form the cornerstone in the success of gene therapy. However, severe deficiencies exist in methods for delivering nucleic acid cargo (e.g., large cargo) in a therapeutically safe and effective manner. Viral delivery mechanisms are frequently used to deliver large nucleic acid cargo in a cell but are tied to safety issues and cannot be used to express the cargo in some cell types. Additionally, subjecting a cell to repeated gene manipulation can affect cell health, induce alterations of cell cycle and render the cell unsuitable for therapeutic use. Advancements are continually sought in the area for efficacious delivery and stabilization of an exogenously introduced genetic material for therapeutic purposes.

### SUMMARY

Provided herein is a pharmaceutical composition comprising a therapeutically effective amount of one or more polynucleic acids, or at least one vector encoding the one or more polynucleic acids, the one or more polynucleic acids comprising: a mobile genetic element comprising a sequence encoding a polypeptide; and an insert sequence, wherein the insert sequence comprises a sequence that is a reverse complement of a sequence encoding an exogenous therapeutic polypeptide, wherein the polypeptide encoded by the sequence of the mobile genetic element promotes integration of the insert sequence into a genome of a cell; and wherein the pharmaceutical composition is substantially non-immunogenic to a human subject.

In some embodiments, the polypeptide encoded by the sequence of the mobile genetic element comprises one or more long interspersed nuclear element (LINE) polypeptides, wherein the one or more LINE polypeptides comprises: human ORF1p or a functional fragment thereof, and human ORF2p or a functional fragment thereof.

In some embodiments, the insert sequence stably integrates and/or is retrotransposed into the genome of a human cell.

In some embodiments, the human cell is an immune cell selected from the group consisting of a T cell, a B cell, a myeloid cell, a monocyte, a macrophage and a dendritic cell.

In some embodiments, the insert sequence is integrated into the genome (i) by cleavage of a DNA strand of a target site by an endonuclease encoded by the one or more polynucleic acids, (ii) via target-primed reverse transcription (TPRT) or (iii) via reverse splicing of the insert sequence into a DNA target site of the genome. In some embodiments, the insert sequence is integrated into the genome at a poly T site using specificity of an endonuclease domain of the human ORF2p. In some embodiments, the poly T site comprises the sequence TTTTTA. In some embodiments, the one or more polynucleic acids comprises homology arms complementary to a target site in the genome. In some embodiments, the insert sequence integrates into: (a) the genome at a locus that is not a ribosomal RNA locus; (b) a gene or regulatory region of a gene of the genome, thereby disrupting the gene or downregulating expression of the gene; (c) a gene or regulatory region of a gene of the genome, thereby upregulating expression of the gene; or (d) the genome and replaces a gene of the genome. In some embodiments, the pharmaceutical composition further comprises (i) one or more siRNAs and/or (ii) an RNA guide sequence or a polynucleic acid encoding the RNA guide sequence, and wherein the RNA guide sequence targets a DNA target site of the genome and the insert sequence is integrated into the genome at the DNA target site of the genome. In some embodiments, the one or more polynucleic acids have a total length of from 3 kb to 20 kb. In some embodiments, the one or more polynucleic acids comprises one or more polyribonucleic acids, one or more RNAs or one or more mRNAs. In some embodiments, the exogenous therapeutic polypeptide is selected from the group consisting of a ligand, an antibody, a receptor, an enzyme, a transport protein, a structural protein, a hormone, a contractile protein, a storage protein and a transcription factor. In some embodiments, the exogenous therapeutic polypeptide is a receptor selected from the group consisting of a chimeric antigen receptor (CAR) and a T cell receptor (TCR). In some embodiments, the one or more polynucleic acids comprises a first expression cassette comprising a promoter sequence, a 5′ UTR sequence, a 3′ UTR sequence and a poly A sequence; wherein: the promoter sequence is upstream of the 5′ UTR sequence, the 5′ UTR sequence is upstream of the sequence of the mobile genetic element encoding a polypeptide, the 3′ UTR sequence is downstream of the insert sequence; and the 3′ UTR is upstream of the poly A sequence; and wherein the 5′ UTR sequence, the 3′ UTR sequence or the poly A sequence comprises a binding site for a human ORF2p or a functional fragment thereof. In some embodiments, the insert sequence comprises a second expression cassette comprising a sequence that is a reverse complement of a promoter sequence, a sequence that is a reverse complement of a 5′ UTR sequence, a sequence that is a reverse complement of a 3′ UTR sequence and a sequence that is a reverse complement of a poly A sequence; wherein: (i) the sequence that is a reverse complement of a promoter sequence is downstream of the sequence that is a reverse complement of a 5′ UTR sequence, (ii) the sequence that is a reverse complement of a 5′ UTR sequence is downstream of the sequence that is a reverse complement of a sequence encoding an exogenous therapeutic polypeptide, (iii) the sequence that is a reverse complement of a 3′ UTR sequence is upstream of the sequence that is a reverse complement of a sequence encoding an exogenous therapeutic polypeptide, and (iv) the sequence that is a reverse complement of a poly A sequence is upstream of the sequence that is a reverse complement of a 3′ UTR sequence and downstream of the sequence of the mobile genetic encoding a polypeptide. In some embodiments, the promoter sequence of the first expression cassette is different from the promoter sequence of the second expression cassette. In some embodiments, the one or more LINE polypeptides comprises a first LINE polypeptide comprising the human ORF1p or functional fragment thereof and a second LINE polypeptide comprising the human ORF2p or functional fragment thereof, wherein the first LINE polypeptide and the second LINE polypeptide are translated from different open reading frames (ORFs). In some embodiments, the one or more polynucleic acids comprises a first polynucleic acid molecule encoding the human ORF1p or functional fragment thereof and a second polynucleic acid molecule encoding the human ORF2p or functional fragment thereof. In some embodiments, the one or more polynucleic acids comprises a 5′ UTR sequence and a 3′ UTR sequence, wherein the 5′ UTR comprises a 5′ UTR from LINE-1 or a sequence with at least 80% sequence identity to ACUCCUCCCCAUCCUCUCCCUCUGUCCCUCUGUCCCUCUGACCCUGCACUGUCCCAGC ACC (SEQ ID NO: 51); and/or the 3′ UTR comprises a 3′ UTR from LINE-1 or a sequence with at least 80% sequence identity to CAGGACACAGCCUUGGAUCAGGACAGAGACUUGGGGGCCAUCCUGCCCCUCCAACCC GACAUGUGUACCUCAGCUUUUUCCCUCACUUGCAUCAAUAAAGCUUCUGUGUUUGGA ACAG (SEQ ID NO: 52). In some embodiments, the sequence encoding the exogenous therapeutic polypeptide does not comprise introns. In some embodiments, the polypeptide encoded by the sequence of the mobile genetic element comprises a C-terminal nuclear localization signal (NLS), an N-terminal NLS or both. In some embodiments, the sequence encoding the exogenous polypeptide is not in frame with a sequence encoding the ORF1p or functional fragment thereof and/or is not in frame with a sequence encoding the ORF2p or functional fragment thereof. In some embodiments, the one or more polynucleic acids comprises a sequence encoding a nuclease domain, a nuclease domain that is not derived from ORF2p, a megaTAL nuclease domain, a TALEN domain, a Cas9 domain, a Cas6 domain, a Cas7 domain, a Cas8 domain, a zinc finger binding domain from an R2 retroelement, or a DNA binding domain that binds to repeat sequences. In some embodiments, the one or more polynucleic acids comprises a sequence encoding the nuclease domain, wherein the nuclease domain does not have nuclease activity or comprises a mutation that reduces activity of the nuclease domain compared to the nuclease domain without the mutation. In some embodiments, the ORF2p or functional fragment thereof lacks endonuclease activity or comprises a mutation selected from the group consisting of S228P and Y1180A, and/or wherein the ORF1p or functional fragment comprises a K3R mutation. In some embodiments, the insert sequence comprises a sequence that is a reverse complement of a sequence encoding two or more exogenous therapeutic polypeptides. In some embodiments, the one or more polynucleic acids comprises one or more polyribonucleic acids, wherein the exogenous therapeutic polypeptide is a receptor selected from the group consisting of a chimeric antigen receptor (CAR) and a T cell receptor (TCR), and wherein the pharmaceutical composition is formulated for systemic administration to a human subject. In some embodiments, the one or more polynucleic acids are formulated in a nanoparticle selected from the group consisting of (i) a lipid nanoparticle and a polymeric nanoparticle; and/or (ii) comprises one or more polynucleic acids selected from the group consisting of glycosylated RNAs, circular RNAs and self-replicating RNAs.

Provided herein is a method, wherein the method is: (i) a method of treating a disease or condition in a human subject in need thereof comprising administering a pharmaceutical composition described herein to the human subject; or (ii) a method of modifying a population of human cells ex vivo comprising contacting a composition to a population of human cell ex vivo, thereby forming an ex vivo modified population of human cells, the composition comprising one or more polynucleic acids, or at least one vector encoding the one or more polynucleic acids, the one or more polynucleic acids comprising: a mobile genetic element comprising a sequence encoding a polypeptide; and an insert sequence, wherein the insert sequence is a reverse complement of a sequence encoding an exogenous therapeutic polypeptide, wherein the ex vivo modified population of human cells is substantially non-immunogenic to a human subject. In some embodiments, the one or more polynucleic acids further comprise (i) a sequence encoding an integrase or a fragment thereof for site directed integration of the insert sequence into the genome and (ii) an integrase genomic landing site sequence that operable by the integrase, wherein the genome landing sequence is greater than 4 consecutive nucleotides long. In some embodiments, the ORF2 and the integrase are on separate polynucleotides. In some embodiments, the ORF2 and the integrase are on a single polynucleotide. In some embodiments, the integrase is not integrated into the genome of the cell. In some embodiments, the integrase is a mutated or truncated recombinant protein. In some embodiments, the integrase genomic landing sequence that is operable by the integrase is greater than 20 nucleotides long, or greater than 30 nucleotides long. In some embodiments, the insert sequence comprises an attachment site operable by the integrase. In some embodiments, the integrase genomic landing site is inserted into the genome using a guide RNA and a Cas system. In some embodiments, the guide RNA, the CAS system and the genomic landing sequence are in a polynucleotide that is separate from the polynucleotide comprising the sequence encoding the LINE 1-ORFs and the insert sequence. In some embodiments, one or more ORF polypeptide sequence comprises a mutation. A method for a site-specific integration of a heterologous genomic insert sequence into the genome of a mammalian cell, the method comprising: (i) introducing into the cell (a) a polynucleotide comprising sequences encoding one or more human retrotransposon elements associated with the heterologous insert sequence, and (b) a polynucleotide comprising sequence encoding a guide RNA, an RNA guided integrase or a fragment thereof and a landing sequence operable by the integrase; (ii) verifying the integration of the heterologous insert sequence into the site of the genome.

Provided herein is a method for site-specific integration of a heterologous genomic insert using a LINE retrotransposon system, wherein the LINE retrotransposon system is modified to incorporate a fragment of an integrase protein that can recognize a genomic landing sequence of greater than 10 consecutive nucleotides long, and wherein the LINE retrotransposon system integrates the heterologous genomic insert into the genomic landing sequence recognized by the fragment of the integrase protein. In some embodiments, the method further comprises a step of incorporating into the genome the genomic landing sequence of greater than 4 consecutive nucleotides long. In some embodiments, the step of incorporating into the genome the genomic landing sequence is performed by an RNA-guided CRISPR-Cas system. In some embodiments, the RNA-guided CRISPR-Cas system has an editing function capable of incorporating a sequence of greater than 4 consecutive nucleotides long into a specific genome site. In some embodiments, the RNA-guided CRISPR-Cas system incorporates an ORF-mRNA binding sequence into a specified location within the genome that has sequence homology to the sequence of the guide RNA. In some embodiments, the insert is about 10 kilobases or greater than 10 kilobases. In some embodiments, the polynucleotide is mRNA.

Provided herein is a method of stably integrating an insert sequence into genomic DNA of a target cell, the method comprising: contacting a composition to the target cell, the composition comprising a polynucleic acid, wherein the polynucleic acid comprises: an insert sequence, wherein the insert sequence comprises a sequence that is a reverse complement of a sequence encoding an exogenous polypeptide, and a mobile genetic element comprising a sequence encoding a polypeptide, wherein the polypeptide encoded by the sequence of the mobile genetic element promotes integration of the insert sequence into genomic DNA; stably integrating the insert sequence into the genomic DNA of the target cell; and expressing an exogenous polypeptide in the target cell, wherein the target cell is a human hepatocyte. In some embodiments, the human hepatocyte is a primary cell. In some embodiments, the human hepatocyte is a from a cultured hepatocyte cell line. In some embodiments, incorporating comprises electroporating under conditions optimum for a human hepatocyte. In some embodiments, the method further comprises culturing the human hepatocyte in vitro after incorporating for about 2 hours, about 3 hours, about 4 hours, about 5 hours, about 6 hours, about 8 hours, about 10 hours or about 24 hours. In some embodiments, the method further comprises introducing the human hepatocyte expressing the exogenous polypeptide into a human subject in need thereof. In some embodiments, at least 2% of the human hepatocytes express the exogenous polypeptide at day 10 after incorporating.

Provided herein is a method of stably integrating an insert sequence into genomic DNA of a target cell, the method comprising: contacting a composition to the target cell, the composition comprising a polynucleic acid, wherein the polynucleic acid comprises: an insert sequence, wherein the insert sequence comprises a sequence that is a reverse complement of a sequence encoding an exogenous polypeptide, and a mobile genetic element comprising a sequence encoding a polypeptide, wherein the polypeptide encoded by the sequence of the mobile genetic element promotes integration of the insert sequence into genomic DNA; stably integrating the insert sequence into the genomic DNA of the target cell; and expressing an exogenous polypeptide in the target cell, wherein the target cell is a human cardiomyocyte. In some embodiments, the human cardiomyocyte is a primary cell. In some embodiments, the human cardiomyocyte is a from a cultured cardiomyocyte cell line. In some embodiments, incorporating comprises electroporating under conditions optimum for a human cardiomyocyte. In some embodiments, the method further comprises culturing the cardiomyocyte in vitro after incorporating for about 2 hours, about 3 hours, about 4 hours, about 5 hours, about 6 hours, about 8 hours, about 10 hours or up to 24 hours. In some embodiments, the method further comprises introducing the human cardiomyocyte expressing the exogenous polypeptide into a human subject in need thereof. In some embodiments, at least 2% of the human cardiomyocytes express the exogenous polypeptide at day 10 after incorporating.

Provided herein is a method of stably integrating an insert sequence into genomic DNA of a target cell, the method comprising contacting a composition to the target cell, the composition comprising a polynucleic acid, wherein the polynucleic acid comprises: an insert sequence, wherein the insert sequence comprises a sequence that is a reverse complement of a sequence encoding an exogenous polypeptide, and a mobile genetic element comprising a sequence encoding a polypeptide, wherein the polypeptide encoded by the sequence of the mobile genetic element promotes integration of the insert sequence into genomic DNA; stably integrating the insert sequence into the genomic DNA of the target cell; and expressing an exogenous polypeptide in the target cell, wherein the target cell is a human retinal pigment epithelial cell. In some embodiments, the human retinal pigment epithelial cell is a primary cell. In some embodiments, the human retinal pigment epithelial is a from a cultured retinal pigment epithelial cell line. In some embodiments, incorporating comprises electroporating under conditions optimum for a human retinal pigment epithelial cell. In some embodiments, the method further comprises culturing the retinal pigment epithelial cell in vitro after incorporating for about 2 hours, about 3 hours, about 4 hours, about 5 hours, about 6 hours, about 8 hours, about 10 hours or up to 24 hours. In some embodiments, the method further comprises introducing the human retinal pigment epithelial cell expressing the exogenous polypeptide into a human subject in need thereof. In some embodiments, at least 2% of the human RPE express the exogenous polypeptide at day 10 after incorporating.

Provided herein is a method of stably integrating an insert sequence into genomic DNA of a target cell, the method comprising contacting a composition to the target cell, the composition comprising a polynucleic acid, wherein the polynucleic acid comprises: an insert sequence, wherein the insert sequence comprises a sequence that is a reverse complement of a sequence encoding an exogenous polypeptide, and a mobile genetic element comprising a sequence encoding a polypeptide, wherein the polypeptide encoded by the sequence of the mobile genetic element promotes integration of the insert sequence into genomic DNA: stably integrating the insert sequence into the genomic DNA of the target cell; and expressing an exogenous polypeptide in the target cell, wherein the target cell is a human neuronal cell. In some embodiments, the human neuronal cell is a primary cell. In some embodiments, the human neuronal cell is a from a cultured neuronal cell line. In some embodiments, incorporating comprises electroporating under conditions optimum for a human neuronal cell. In some embodiments, the method further comprises culturing the neuronal cell in vitro after incorporating for about 2 hours, about 3 hours, about 4 hours, about 5 hours, about 6 hours, about 8 hours, about 10 hours or up to 24 hours. In some embodiments, the method further comprises introducing the human neuronal cell expressing the exogenous polypeptide into a human. In some embodiments, at least 2% of the human neuronal cells express the exogenous polypeptide at day 10 after incorporating. In some embodiments, the insert sequence is a human insert sequence. In some embodiments, the exogenous polypeptide is an exogenous therapeutic polypeptide. In some embodiments, the exogenous polypeptide is an exogenous human polypeptide. In some embodiments, the polypeptide encoded by the sequence of the mobile genetic element promotes integration of the insert sequence into genomic DNA via target-primed reverse transcription (TPRT). In some embodiments, the polynucleic acid is an mRNA or an mRNA molecule. In some embodiments, the mobile genetic element comprises a human LINE 1 retrotransposon element. In some embodiments, the ORF2p is selected from a non-human species. In some embodiments, the ORF2p selected from a non-human species is further modified to enhance retrotransposition efficiency and/or translation efficiency. In some embodiments, the cell is an immune cell, a hepatocyte, a cardiomyocyte, a retinal pigment epithelial cell or a neuron. In some embodiments, the ORF2p comprises an nuclear localization sequence (NLS). In some embodiments, the ORF2p comprises at least 2 NLSs that are the same or different. In some embodiments, the NLS is N-terminal to a sequence encoding ORF1p, ORF2p or both. In some embodiments, the NLS is C-terminal to a sequence encoding ORF1p, ORF2p or both. In some embodiments, the NLS is from SV40. In some embodiments, the NLS is from nucleoplasmin. In some embodiments, a first NLS of the at least 2 NLSs is from SV40 and a second NLS of the at least 2 NLSs is from nucleoplasmin. In some embodiments, a first and a second NLS of the at least 2 NLSs are from SV40. In some embodiments, a first and a second NLS of the at least 2 NLSs are from nucleoplasmin. In some embodiments, each of the at least 2 NLSs are the same.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “FIG.” herein), of which:

FIG. 1A illustrates a general mechanism of action of retrotransposons. (I) is a schematic representing the overall lifecycle of an autonomous retrotransposon. (II) LINE-1 retrotransposon comprises LINE-1 elements, which encode two proteins ORF1p and ORF2p that are expressed as mRNAs. The bicistronic mRNA is translated into the two proteins, and when ORF2p is translated by a read-through event by the ribosome, it binds the 3′end of its own mRNA through the poly A tail (III). ORF2p cleaves at a consensus sequence TAAAA, where the poly A at the 3′ end of the mRNA hybridizes and primes the reverse transcriptase activity of the ORF2 protein. The protein reverse-transcribes the mRNA back into DNA leading to an insertion of the LINE-1 sequence back into a new location in the genome (IV).

FIG. 1B is an illustration of a schematic diagram of an mRNA construct that comprises a genetic payload (left) that can be designed for integration into the genome (right).

FIG. 1C illustrates various exemplary designs for integrating an mRNA encoding a transgene into the genome of a cell. GFP shown here in a box is an exemplary transgene.

FIG. 1D illustrates various exemplary designs for integrating an mRNA encoding a transgene into the genome of a cell. GFP shown here in a box is an exemplary transgene.

FIG. 1E is an illustration a schematic of the LINE-1 retrotransposition cycle showing the mechanism of action of the LINE transposons and introduction of a transgene cargo into a retrotransposon cite. LINE-1 retrotransposons are genomic sequences that encode for two proteins, ORF1 and ORF2. These elements are transcribed and translated into proteins that form an RNA-protein complex with the LINE-1 mRNA, ORF1 trimers, and ORF2, a reverse-transcriptase endonuclease. This complex translocates back into the nuclease where it cleaves DNA at a 5′-TTTT N-3′ motif and is primed for reverse-transcription of the LINE-1 RNA by the ORF2 protein by making an RNA-DNA hybrid with the poly A tail of the mRNA and the resected cleaved DNA. Reverse-transcription of the LINE-1 into cDNA leads to a new LINE-1 integration event.

FIG. 2A illustrates three exemplary designs for expressing an exemplary transgene GFP by stably incorporating the sequence encoding GFP using the constructs. Expected GFP expression levels at 72 hours are shown on the right side.

FIG. 2B illustrates three exemplary designs for expressing an exemplary transgene GFP by stably incorporating the sequence encoding RFP, RFP and GFP or ORF2p and GFP using the constructs. Expected GFP and RFP expression levels at 72 hours are shown on the right side.

FIG. 3A illustrates an exemplary diagram of conventional circRNA structure and formation.

FIG. 3B illustrates two views of an exemplary RL-GAAA tectoRNA motif designs. FIG. 3B discloses SEQ ID NOS 111-112, respectively, in order of appearance.

FIG. 3C illustrates exemplary structures of chip-flow piece RNAs as platforms for testing potential tectoRNA.

FIG. 4A illustrates an exemplary schematic showing ORF2p binding to an ORF2 poly A region.

FIG. 4B illustrates an exemplary schematic showing how a fusion of ORF2p with an MS2 RNA binding domain binds to an MS2 binding RNA sequence in the 3′UTR of an mRNA encoding the ORF2 an increase specificity.

FIG. 4C illustrates exemplary designs of retrotransposon systems for stably integrating a nucleic acid into the genome of a cell at specific sites. The upper panel shows a design using an ORFp2-MegaTAL DNA binding domain fusion where the DNA binding and endonuclease activity of ORF2p is mutated to be inactive. The middle panel shows a chimeric ORF2p where the endonuclease domain has been replaced with a high specificity and high-fidelity nuclease domain of another protein. The lower panel shows a fusion of a DNA binding domain of a heterologous protein with ORF2p such that the fusion protein binds to ORF2 binding site as well additional DNA sequences in the vicinity of the ORF2 site.

FIG. 5 illustrates exemplary constructs (I)-(X) for integrating an mRNA encoding a transgene into the genome of a cell.

FIG. 6A illustrates an exemplary construct with a sequence encoding ORF1p for integrating an mRNA encoding a transgene into the genome of a cell.

FIG. 6B illustrates an exemplary construct without a sequence encoding ORF1p for integrating an mRNA encoding a transgene into the genome of a cell.

FIG. 7A illustrates exemplary methods of improving mRNA half-life by inhibiting degradation by 5′-3′ exonucleases, such as XRN1, or 3′-5′ exosomal degradation, by introducing structures corresponding to a G-quadruplex, or, a pseudoknot (SEQ ID NO: 113) in the 5′UTR; and/or xrRNAs, a triplex motifs (SEQ ID NOS: 74, 54, and 53, respectively, in order of appearance) and/or a non-A nucleotide residues in the 3′UTR.

FIG. 7B illustrates an exemplary schematic of a myeloid cell expressing a transgene encoding a chimeric receptor that binds a cancer cell and induces anti-cancer activity.

FIG. 7C shows expected results of introducing bulk or purified RNA encoding a chimeric receptor that binds a cancer cell as described in FIG. 7B on increased and prolonged expression of the chimeric receptors.

FIG. 8A shows an exemplary plasmid design and expected LINE-1 mRNA transcript with a cargo nucleic acid sequence. The plasmid has a LINE-1 sequence (comprising ORF1 and ORF2 protein encoding sequences) and a cargo sequence which is a nucleic acid sequence encoding GFP, where the coding sequence of GFP is interrupted with an intron. The GFP is not expressed until the sequence is integrated in the genome and the intron is spliced.

FIG. 8B shows exemplary results showing successful integration of the mRNA transcript encoded by the plasmid shown in FIG. 8A and expression of GFP relative to mock-transfected cells (fold increase in mean fluorescence intensity of GFP positive cells is shown). Mock transfected cells were transfected by the vector lacking the GFP cargo sequence.

FIG. 8C shows exemplary flow cytometry results from the results shown in FIG. 8B.

FIG. 9A shows an exemplary plasmid design and expected LINE-1 mRNA transcript with a cargo nucleic acid sequence. The plasmid has a LINE-1 sequence (comprising ORF1 and ORF2 protein encoding sequences) and a cargo sequence which is a nucleic acid sequence encoding a recombinant chimeric fusion receptor protein (ATAK receptor) that has extracellular region capable of binding to CD5 and an intracellular region comprising an FCR intracellular domain and a PI3 kinase recruitment domain. The coding sequence of the ATAK receptor is interrupted with an intron.

FIG. 9B shows exemplary results showing successful integration of the mRNA transcript encoded by the plasmid shown in FIG. 9A and expression of ATAK relative to mock-transfected cells (fold increase in mean fluorescence intensity of ATAK positive cells is shown). Mock transfected cells were transfected by the vector lacking the ATAK cargo sequence. Expression of ATAK receptor protein was detected by binding with a labeled CD5 antibody.

FIG. 9C shows exemplary flow cytometry results from the results shown in FIG. 9B.

FIG. 10A shows an exemplary plasmid design and expected LINE-1 mRNA transcript with a cargo nucleic acid sequence. The plasmid has a LINE-1 sequence (comprising ORF1 and ORF2 protein encoding sequences) and a cargo sequence which is a nucleic acid sequence encoding a recombinant chimeric fusion receptor protein (ATAK receptor) followed by a T2A self-cleavage sequence followed by a split GFP sequence (all in a reverse orientation relative to the LINE-1 sequence). The coding sequence of the GFP is interrupted with an intron. Expected mRNA after reverse transcription and integration of the cargo are depicted.

FIG. 10B shows exemplary results showing successful integration of the mRNA transcript encoded by the plasmid shown in FIG. 10A and expression of ATAK-T2A-GFP relative to mock-transfected cells (fold change in GFP and ATAK double positive cells is shown). Mock transfected cells were transfected by the vector lacking the ATAK cargo sequence. Expression of ATAK receptor protein was detected by binding with a labeled CD5 antibody.

FIG. 10C shows representative flow cytometry data from two separate experimental runs for expression of both GFP and CD5 binder (ATAK) using the experimental setup shown in FIG. 10A.

FIG. 10D shows representative flow cytometry data from two separate experimental runs for expression of both GFP and CD5 binder (ATAK) using the experimental setup shown in FIG. 10A.

FIG. 11A shows exemplary mRNA constructs for retrotransposition-based gene delivery. The ORF1 and ORF2 sequences are in two difference mRNA molecules. The ORF2p (ORF2) coding mRNA comprises and inverted GFP coding sequence.

FIG. 11B depicts exemplary data showing expression of GFP (fold increase in mean fluorescence intensity of GFP positive cells is shown) upon electroporating both ORF1-mRNA and ORF2-FLAG-GFPai mRNA normalized to electroporation of ORF2-FLAG-GFPai mRNA only.

FIG. 12A depicts exemplary data showing expression of GFP (fold increase in mean fluorescence intensity of GFP positive cells is shown) upon electroporating ORF1-mRNA and ORF2-FLAG-GFPai mRNA at different amounts. Fold increase is relative to 1× ORF2-GFPao and 1× ORF1 mRNA.

FIG. 12B shows an exemplary fluorescent microscopy image of GFP⁺ cells following electroporation of the mRNA depicted in FIG. 11A.

FIG. 13A shows exemplary mRNA constructs where the ORF1 and ORF2 sequences are in two difference mRNA molecules (top panel) and a LINE-1 mRNA transcript comprising ORF1 and ORF2 protein encoding sequences on a single mRNA molecule (bottom panel) for gene delivery. mRNA contains the bicistronic ORF1 and ORF2 sequence with a CMV-GFP sequence in the 3′UTR going from 3′-5′. Upon retrotransposition of the delivered ORF2-cmv-GFP antisense (LINE-1 mRNA), cells are expected to express GFP.

FIG. 13B depicts exemplary data showing expression of GFP (fold increase in mean fluorescence intensity of GFP positive cells is shown) upon electroporating the constructs depicted in FIG. 13A.

FIG. 14A shows an exemplary experimental design for testing whether multiple electroporations increases retrotransposition efficiency. HEK293T cells were electroporated every 48 hours with the Maxcyte system and assessed for GFP positive cells using flow after culturing for 24-72 hrs.

FIG. 14B depicts exemplary data showing expression of GFP at the indicated times (fold increase in mean fluorescence intensity of GFP positive cells is shown) upon electroporating 1-5 times according to FIG. 14A.

FIG. 15A depicts exemplary constructs to enhance retrotransposition via mRNA delivery. In one construct a nuclear localization signal (NLS) sequence is fused to the C terminus of the ORF2 sequence (ORF2-NLS fusion). In one construct a Minke whale ORF2 sequence was used in place of the human ORF2. In one construct a minimal sequence of the Alu element (AJL-H33delta) is inserted in the 3′UTR of the LINE-1 sequence. In one construct MS2 hairpins are inserted in the 3′UTR of the LINE-1 sequence and an MS2 hairpin binding protein (MCP) sequence is fused to the ORF2 sequence.

FIG. 15B depicts exemplary data showing expression of GFP (fold increase in mean fluorescence intensity of GFP positive cells is shown) using the constructs depicted in FIG. 15A.

FIG. 16A shows exemplary plasmid constructs where the ORF1 and ORF2 sequences are in two difference plasmid molecules (top panel) and a plasmid encoding a LINE-1 mRNA transcript comprising ORF1 and ORF2 protein encoding sequences on a single mRNA molecule with various replacements of the inter-ORF sequence between ORF1 and ORF2 (bottom panel) for gene delivery.

FIG. 16B depicts exemplary data showing expression of GFP (fold increase in mean fluorescence intensity of GFP positive cells is shown) using the constructs depicted in FIG. 16A.

FIG. 17A depicts an exemplary plasmid construct encoding a LINE-1 mRNA transcript comprising ORF1 and ORF2 protein encoding sequences on a single mRNA molecule with a GFP sequence (top panel) and an exemplary LINE-1 mRNA transcript comprising ORF1 and ORF2 protein encoding sequences on a single mRNA molecule with a GFP sequence.

FIG. 17B depicts exemplary data showing expression of GFP (fold increase in mean fluorescence intensity of GFP positive cells is shown) in Jurkat cells using the constructs depicted in FIG. 17A. The plasmid construct was transfected, and the mRNA construct was electroporated.

FIG. 18A shows an exemplary plasmid design and expected LINE-1 mRNA transcript with a cargo nucleic acid sequence. The plasmid has a LINE-1 sequence (comprising ORF1 and ORF2 protein encoding sequences) and a cargo sequence which is a nucleic acid sequence encoding a recombinant chimeric fusion receptor protein (ATAK receptor) followed by a T2A self-cleavage sequence followed by a split GFP sequence (all in a reverse orientation relative to the LINE-1 sequence). The coding sequence of the GFP is interrupted with an intron. Expected mRNA after reverse transcription and integration of the cargo are depicted.

FIG. 18B shows exemplary results showing successful integration of the mRNA transcript encoded by the plasmid shown in FIG. 10A and expression of ATAK-T2A-GFP relative to mock-transfected cells (fold change in GFP and ATAK double positive cells is shown) in a myeloid cell line (THP-1). Data represents expression at 6 days post transfection, normalized over mock plasmid transfected cells wherein the mock plasmid does not have GFP coding sequence.

FIG. 19 illustrates an exemplary experimental set up for cell synchronization. A heterogenous cell population is sorted based on cell cycle stage, prior to delivery of an exogenous nucleic acid. Cell cycle synchronization is expected to result in higher expression and stabilization of the exogenous nucleic acid delivered. If cells are not homogeneous after cell sorting, then cells can be further incubated with a suitable agent that arrests cell cycle at a stage.

FIG. 20 illustrates an exemplary method for increasing retrotransposon efficiency by inducing DNA double stranded breaks, with or without inhibiting DNA repair pathways, such as by inducing DNA ligase inhibitor SCR7 or inhibiting host surveillance proteins, for example, using miRNA to HUSH complex TASOR protein.

FIG. 21 illustrates exemplary constructs for integrating an mRNA encoding a transgene into the genome of a cell.

FIG. 22 illustrates exemplary constructs for integrating an mRNA encoding a transgene into the genome of a cell.

FIG. 23 illustrates exemplary constructs for integrating an mRNA encoding a transgene into the genome of a cell.

FIG. 24 illustrates exemplary constructs for integrating an mRNA encoding a transgene into the genome of a cell.

FIG. 25 illustrates exemplary constructs for integrating an mRNA encoding a transgene into the genome of a cell.

FIG. 26 illustrates exemplary constructs for integrating an mRNA encoding a transgene into the genome of a cell.

FIG. 27 illustrates exemplary constructs for integrating an mRNA encoding a transgene into the genome of a cell.

FIG. 28 illustrates exemplary constructs for integrating an mRNA encoding a transgene into the genome of a cell.

FIG. 29 illustrates exemplary retrotransposon constructs (left) with a 2.4 kb cargo with a general mechanism of action of the retrotransposon, and a representative data (right) for expression of a fluorescent GFP marker encoded by the cargo from a nucleic acid sequence integrated into the genome in HEK293 cells. Placement of an antisense GFP gene split with an intron in the sense direction and a promoter sequence in the 3′UTR of the LINE-1 leads to reconstitution and retrotransposition of the GFP cargo. GFP expression in 293T cells transfected with the construct shown on the left, as measured by flow cytometry (right) and quantitated bar graphs (bottom left). Data collected 35 days after doxycycline induction of the ORF.

FIG. 30 illustrates exemplary retrotransposon constructs (left) with a 3.0 kb cargo comprising a membrane protein (CD5 binder chimeric antigen receptor, CD5-CAR), and are presentative flow cytometry data for expression of the CD5 binder (right) from the nucleic acid sequence integrated into the genome in HEK293 cells. % of CD5 binder positive (+) cells is indicated in the inset.

FIG. 31 illustrates an exemplary retrotransposon construct (top) with a 3.7 kb cargo comprising a membrane protein (CD5 binder chimeric antigen receptor, CD5-CAR and a GFP separated by an auto-cleavable T2A element), and a representative flow cytometry data (bottom) demonstrating the expression of the CD5 binder and GFP.

FIG. 32 illustrates an exemplary retrotransposon construct (top) with a 3.9 kb cargo comprising a membrane protein (HER2 binder chimeric antigen receptor, and a GFP separated by an auto-cleavable T2A element), and a representative flow cytometry data (bottom) demonstrating the expression of the HER2 binder and GFP.

FIG. 33A shows exemplary data for delivery of retrotransposon elements delivered as mRNA.

FIG. 33B shows schematic diagram showing a trans and a cis mRNA design for delivery of LINE1 mRNA with GFP cargo (top panel). Representative results of electroporation of 293T cells with trans mRNAs with separate ORF1 and ORF2 mRNAs. 293T cells were electroporated with 100 μg/mL of mRNA either with ORF2 alone, ORF1+ ORF2 mRNAs, each at 100 μg/mL, or a GFP-encoding mRNA with the same 5′ and 3′UTRs as the ORF1 mRNA (left panel of data plots). Retrotransposition events result in GFP-positive cells. Cells were assayed for GFP fluorescence by flow cytometry 4 days and 10 days post-electroporation. Mock electroporated cells serve as the negative control population for gating. Bar graph on the right shows results from a representative experiment indicating titration of trans mRNAs and cis ORF1 and ORF2 containing mRNA concentration during electroporation. Trans mRNAs solid bars and cis mRNA stripes. 20× is 2000 μg/mL in the electroporation reaction.

FIG. 33C shows titration of the ORF1 and ORF2-GFPai trans mRNAs. Increasing the concentration separately and together during the electroporation to 200 μg/mL increases retrotransposition of the GFP gene cargo.

FIG. 33D illustrates an exemplary data for the different constructs indicated above each flow cytometry data plot in the figure, the top panel on day 4, and the bottom panel on day 13. Right hand figures illustrate light and fluorescent microscopic images of a the GFP expressing cells in culture. Copies of integrated cargo per construct is demonstrated in the bottom right at day 13. qPCR assay for genomic DNA integration from different LINE-1 plasmid transfected, LINE-1 mRNA (retro-mRNA), and ORF1 and ORF2-GFP mRNA electroporated cells is shown. Two qPCR primer-probe sets were used, one for the housekeeping gene RPS30 and the other for the GFP gene. Plasmid-transfected cells use a plasmid that does not contain and SV40 maintenance sequence. Integration per cell is calculated from determining copy numbers per samples through interpolation of a standard curve of plasmid and genomic DNA, and normalizing for the two copies of RPS30 per 293T cell. Error bars denote standard deviation of three technical replicate measurements.

FIG. 34 illustrates exemplary retrotransposon construct (left) and expression data (right) in the indicated cell lines.

FIG. 35 illustrates flow cytometry data showing expression of LINE 1 GFP constructs in K562, 293T and THP1 cells (upper panel); and number of integrations of LINE-2-GFP mRNA per cell in K562 and THP-1 cell lines (lower panel).

FIG. 36 illustrates flow cytometry data showing expression of LINE 1 GFP constructs in primary T cells (left). Integrations per cell are indicated in the graph on the right. Data was collected on day 6 after electroporation.

FIG. 37A shows a schematic of activation, culture times, electroporation, and GFP expression assay of isolated primary T cells.

FIG. 37B illustrates flow cytometry data showing expression of LINE 1 GFP mRNA constructs in primary T cells at the indicated concentrations and before and after freeze-thaw as indicated in the figure. Integrations per cell is shown in the bar diagram. GFP expression using a retro-mRNA electroporation with a GFP cargo. GFP expression was assayed 4 days post electroporation and 15 days of culturing post electroporation. Primary T cells were cryo-preserved and thawed during this time. qPCR integration assay for GFP integration. Genomic DNA from the 20× sample was isolated and assayed for copies of GFP.

FIG. 38 demonstrates a summary of results of retrotransposon integration and expression across cell types.

FIG. 39 shows various applications of the technology described herein, including but not limited to use of CART cells, NK cells, neurons and other cells for cell therapy, and use of in vivo applications in including but not limited to gene therapy, gene editing, transcription regulation, and genome engineering.

FIG. 40 depicts exemplary flow cytometry data showing sorting and enriching GFP+ 293T cells electroporated with 2000 ng/μL LINE 1-GFP mRNA. The first panel shows flow cytometry data for mock electroporated cells in the absence of LINE1-GFP mRNA. The second panel shows flow cytometry data collected 5 days post electroporation for unsorted cells electroporated with LINE1-GFP mRNA. The GFP+ cells from the second panel were sorted and the flow cytometry data are shown in the third panel. The GFP+ cells from the third panel were cultured for 9 days post sorting and resorted using 10′ or 10∝GFP fluorescence intensity gate. The fourth panel shows flow cytometry data for cells resorted using GFP+at 10′GFP gate collected 4 days after resorting. The fifth panel shows flow cytometry data for cells resorted using GFP+at 10′GFP gate collected 4 days after resorting.

FIG. 41A shows a standard curve for GFP (NB2 plasmid) and a housekeeping gene (FAU) for evaluating genomic integration of GFP-encoding nucleic acid per cell using quantitative PCR.

FIG. 41B shows results of an exemplary graph depicting interpolation of the standard curves of FIG. 41A for quantitation of genomic integration.

FIG. 41C shows the number of the GFP gene integrated into genome of 293T cells following LINE1-GFP mRNA electroporation and double sorting as shown in FIG. 40. The average number of GFP integrations per cell when gated at 10′GFP+ cells and at 10∝GFP+ cells according to qPCR are shown.

FIG. 42 depicts exemplary flow cytometry data showing GFP+ 293T cells electroporated with the indicated titrated amounts of LINE1-GFP mRNA, in ng/μL in electroporation solution, after culturing for 3 days post-electroporation.

FIG. 43 depicts exemplary flow cytometry data showing GFP+ 293T cells electroporated with the indicated titrated amounts of LINE1-GFP mRNA, in ng/μL in electroporation solution, after culturing for 5 days post-electroporation.

FIG. 44 depicts exemplary flow cytometry data showing GFP+ 293T cells electroporated with the indicated titrated amounts of LINE1-GFP mRNA, in ng/μL in electroporation solution, after culturing for 7 days post-electroporation.

FIG. 45 shows a graph of the number of GFP integrations per genome of 293T cells electroporated with the indicated titrated amounts of LINE1-GFP mRNA, in ng/μL in electroporation solution, according to qPCR after culturing for 3, 5 or 7 days post-electroporation according to FIGS. 42-44 (top) and a graph of the integration kinetics (bottom) according to the data from FIGS. 42-44.

FIG. 46 depicts exemplary flow cytometry data (right) showing GFP+K562 cells electroporated with the indicated titrated amounts of LINE1-GFP mRNA, in ng/μL in electroporation solution, after culturing for 6 days post-electroporation, and a graph of the number of GFP integrations per genome according to qPCR (left).

FIG. 47 depicts exemplary flow cytometry data (top) showing GFP+ human primary monocytes electroporated with the indicated titrated amounts of LINE1-GFP mRNA after culturing for 3 days post-electroporation, and a graph of the number of GFP integrations per genome according to qPCR (bottom).

FIG. 48 depicts exemplary flow cytometry data (bottom) showing GFP+ 293T cells electroporated with 2000 ng/μL LINE1-GFP mRNA and 100 ng/μL, 200 ng/μL or 300 ng/μL of an siRNA targeting BRCA1 (siBRCA1) after culturing for 4 days post-electroporation and a graph of the number of GFP integrations per genome according to qPCR (top).

FIG. 49 depicts exemplary flow cytometry data (bottom) showing GFP+ 293T cells electroporated with 2000 ng/μL LINE1-GFP mRNA and 100 ng/μL of an siRNA targeting RNASEL (siRNASEL), ADAR1 (siADAR1), or ADAR2 (siADAR2) after culturing for 6 days post-electroporation and a graph of the number of GFP integrations per genome according to qPCR (top).

FIG. 50 depicts exemplary flow cytometry data (bottom) showing GFP+ 293T cells electroporated with 2000 ng/μL LINE1-GFP mRNA and 100 ng/μL of an siRNA targeting APOBEC3C (siAPOBEC3C) or FAM208A (siFAM208A) after culturing for 6 days post-electroporation and a graph of the number of GFP integrations per genome according to qPCR (top).

FIG. 51 depicts exemplary flow cytometry data (bottom) showing GFP+ 293T cells electroporated with 1000 ng/μL or 1500 ng/μL LINE 1-GFP mRNA and an siRNA cocktail with 25 ng/μL, 50 ng/μL or 75 ng/μL of each siRNA targeting RNASEL (siRNASEL), ADAR1 (siADAR1), ADAR2 (siADAR2) and BRCA1 (siBRCA1) after culturing for 6 days post-electroporation and a graph of the number of GFP integrations per genome according to qPCR (top).

FIG. 52 depicts exemplary flow cytometry data (bottom) showing GFP+K562 cells electroporated with 1000 ng/μL LINE1-GFP mRNA and an siRNA cocktail with 25 ng/μL, 50 ng/μL or 75 ng/μL of each siRNA targeting RNASEL (siRNASEL), ADAR1 (siADAR1), ADAR2 (siADAR2) and BRCA1 (siBRCA1) after culturing for 5 days post-electroporation and a graph of the number of GFP integrations per cell according to qPCR (top).

FIG. 53 depicts a schematic showing exemplary locations of extraneous nuclear localization sequences (NLS) and exemplary ORF1p and ORF2p mutations of an exemplary LINE1-GFP mRNA construct.

FIG. 54A depicts a schematic showing an exemplary LINE1-GFP construct in which an NLS was inserted at the N-terminal end of the sequence encoding ORF1.

FIG. 54B depicts a bar graph showing GFP integrations per cell on day 4 post electroporation of the indicated constructs into 293T cells.

FIG. 54C depicts exemplary flow cytometry showing GFP+ 293T cells on day 4 post electroporation of the indicated constructs.

FIG. 55A depicts a schematic showing an exemplary LINE1-GFP construct in which an NLS was inserted at the C-terminal end of the sequence encoding ORF1.

FIG. 55B depicts a bar graph showing GFP integrations per cell on day 4 post electroporation of the indicated constructs into 293T cells.

FIG. 55C depicts exemplary flow cytometry showing GFP+ 293T cells on day 4 post electroporation of the indicated constructs.

FIG. 56A depicts a schematic showing an exemplary LINE1-GFP construct in which an NLS was inserted at the N-terminal end of the sequence encoding ORF2.

FIG. 56B depicts a bar graph showing GFP integrations per cell on day 4 post electroporation of the indicated constructs into 293T cells.

FIG. 56C depicts exemplary flow cytometry showing GFP+ 293T cells on day 4 post electroporation of the indicated constructs.

FIG. 57A depicts a schematic showing an exemplary LINE 1-GFP construct in which an NLS and a linker was inserted at the N-terminal end of the sequence encoding ORF2.

FIG. 57B depicts a bar graph showing GFP integrations per cell on day 5 post electroporation of the indicated constructs into 293T cells.

FIG. 57C depicts exemplary flow cytometry showing GFP+ 293T cells on day 5 post electroporation of the indicated constructs.

FIG. 58A depicts a schematic showing an exemplary LINE1-GFP construct in which an NLS was inserted at the C-terminal end of the sequence encoding ORF2.

FIG. 58B depicts a bar graph showing GFP integrations per cell on day 5 post electroporation of the indicated constructs into 293T cells.

FIG. 58C depicts exemplary flow cytometry showing GFP+ 293T cells on day 5 post electroporation of the indicated constructs.

### DETAILED DESCRIPTION

The present invention arises in part from the exciting discovery that a polynucleotide could be designed and developed to accomplish transfer and integration of a genetic cargo (e.g., large genetic cargo) into the genome of a cell. In some embodiments, the polynucleotide comprises (i) a genetic material for stable expression, and (ii) a self-integrating genomic integration machinery that allows stable integration of the genetic material into a cell by non-viral means, that is both safe and efficacious. Moreover, the genetic material may be integrated at a locus other than a ribosomal locus; the genetic material may be integrated site-specifically; and/or the integrated genetic material appear to express without triggering a cell's natural silencing machinery.

Clustered Regularly-Interspaced Short Palindromic Repeats (CRISPR) revolutionized the molecular biology field and has developed into a potent gene editing too. It utilizes homology-directed repair (HDR) and can be directed to a genomic site. CRISPR/Cas9 is a naturally occurring RNA-guided endonuclease. While the CRISPR/Cas9 system has demonstrated great promise for site-specific gene editing and other applications, there are several factors that influence its efficacy which must be addressed, especially if it is to be used for in vivo human gene therapy. These factors include target DNA site selection, sgRNA design, off-target cutting, incidence/efficiency of HDR vs. NHEJ, Cas9 activity, and the method of delivery. Delivery remains the major obstacle for use of CRISPR for in vivo applications. Zinc finger nucleases ZFNs are a fusion protein of Cys2-His2 zinc finger proteins (ZFPs) and a non-specific DNA restriction enzyme derived from FokI endonucleases. Challenges with ZFPs include design and engineering of the ZFP for high-affinity binding of the desired sequence, which is non-trivial. Also, not all sequences are available for ZFP binding, so site selection is limited. Another significant challenge is off-target cutting. Transcription activator-like effector nucleases (TALENs) are a fusion protein comprised of a TALE and a FokI nuclease. While off-target cutting remains a concern, TALENs have been shown in one side-by-side comparison study to be more specific and less cytotoxic than ZFNs. However, TALENs are substantially larger, and the cDNA encoding TALEN only is 3kb. This makes delivery of a pair of TALENs more challenging than a pair of ZFNs due to delivery vehicle cargo size limitations. Further, packaging and delivery of TALENs in some viral vectors may be problematic due to the high level of repetition in the TALENs sequence. A mutant Cas9 system, a fusion protein of inactive dCas9 and a FokI nuclease dimer increase specificity and reduce off-target cutting, the number of potential target sites is lower due to PAM and other sgRNA design constraints.

The present invention addresses the problems described above by providing new, effective and efficient compositions comprising transposon-based vectors for providing therapy, including gene therapy, to animals and humans. The present invention provides methods of using these compositions for providing therapy to animals and humans. These transposon-based vectors can be used in the preparation of a medicament useful for providing a desired effect to a recipient following administration. Gene therapy includes, but is not limited to, introduction of a gene, such as an exogenous gene, into an animal using a transposon-based vector. These genes may serve a variety of functions in the recipient such as coding for the production of nucleic acids, for example RNA, or coding for the production of proteins and peptides. The present invention can facilitate efficient incorporation of the polynucleotide sequences, including the genes of interest, promoters, insertion sequences, poly A and any regulatory sequences. The invention is based on the finding that human LINE-1 elements are capable of retrotransposition in human cells as well as cells of other animal species and can be manipulated in a versatile manner to achieve efficient delivery and integration of a genetic cargo into the genome of a cell. Such LINE-1 elements have a variety of uses in human and animal genetics including, but not limited to, uses in diagnosis and treatment of genetic disorders and in cancer. The LINE-1 elements of the invention are also useful for the treatment of various phenotypic effects of various diseases. For example, LINE-1 elements may be used for transfer of DNA encoding anti-tumorigenic gene products into cancer cells. Other uses of the LINE-1 elements of the invention will become apparent to the skilled artisan upon a reading of the present specification.

In general, a human LINE-1 element comprises a 5′ UTR with an internal promoter, two non-overlapping reading frames (ORF1 and ORF2), a 200 bp 3′ UTR and a 3′ poly A tail. The LINE-1 retrotransposon can also comprise an endonuclease domain at the LINE-1 ORF2 N-terminus. The finding that LINE-1 encodes an endonuclease demonstrates that the element is capable of autonomous retrotransposition. LINE-1 is a modular protein that contains non-overlapping functional domains which mediate its reverse transcription and integration. In some embodiments, the sequence specificity of the LINE-1 endonuclease itself can be altered or the LINE-1 endonuclease can be replaced with another site-specific endonuclease.

The LINE-1 retrotransposon may be manipulated using recombinant technology to comprise and/or be contiguous with, other nucleic acid elements which render the retrotransposon suitable for insertion of substantial lengths (up to 1 kb, or greater than 1 kb, e.g. greater than 5, 6, 7, 8, 9, or 10 kb) of heterologous or homologous nucleic acid sequence into the genome of a cell. The LINE-1 retrotransposon may also be manipulated using the same type of technology such that insertion of the nucleic acid sequence of heterologous or homologous nucleic acid into the genome of a cell is site-directed (site into which such DNA is inserted is known). Alternatively, the LINE-1 retrotransposon may be manipulated such that the insertion site of the DNA is random. The retrotransposon may also be manipulated to effect insertion of a desired DNA sequence into regions of DNA which are normally transcriptionally silent, wherein the DNA sequence is expressed in a manner such that it does not disrupt the normal expression of genes in the cell. In some embodiments, the integration or retrotransposition is in the trans orientation. In some embodiments, the integration or retrotransposition occurs in the cis orientation.

Since LINE-1 is native to human cells, when the constructs are placed into human cells, they should not be rejected by the immune system as foreign. In addition, the mechanism of LINE-1 retro-integration ensures that only one copy of the gene is integrated at any specific chromosomal location.

Accordingly, there is a copy number control built into the system. In contrast, gene transfer procedures using ordinary plasmids offer little or no control regarding copy number and often result in complex arrays of DNA molecules tandemly integrated into the same genomic location.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, may be used interchangeably. These terms may convey that any combination is specifically contemplated. Solely for illustrative purposes, the following phrases “A, B, and/or C” or “A, B, C, or any combination thereof” may mean “A individually; B individually; C individually; A and B; B and C; A and C; and A, B, and C.” The term “or” may be used conjunctively or disjunctively unless the context specifically refers to a disjunctive use.

The term “about” or “approximately” may mean within an acceptable error range for the particular value as determined by one of ordinary skill in the art, which will depend in part on how the value is measured or determined, i.e., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Alternatively, particularly with respect to biological systems or processes, the term may mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification may be implemented with respect to any method or composition of the present disclosure, and vice versa.

Furthermore, compositions of the present disclosure may be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

Applications of the present disclosure encompasses, but are not limited to methods and compositions related to expression of an exogenous nucleic acid in a cell. In some embodiments, the exogenous nucleic acid is configured for stable integration in the genome of a cell, such as a myeloid cell. In some embodiments, the stable integration of the exogenous nucleic acid may be at specific targets within the genome. In some embodiments, the exogenous nucleic acid comprises one or more coding sequences. In some embodiments, the exogenous nucleic acid may comprise one or more coding comprising a nucleic acid sequence encoding an immune receptor. In some embodiments, the present disclosure provides methods and compositions for a stable incorporation of a nucleic acid encoding a transmembrane receptor implicated in an immune response function (e.g. a phagocytic receptor or synthetic chimeric antigen receptor) into human macrophage or dendritic cell or a suitable myeloid cell or a myeloid precursor cell. An exogenous nucleic acid can refer to a nucleic acid that was not originally in a cell and is added from outside the cell, irrespective of whether it comprises a sequence that may already be present in the cell endogenously. An exogenous nucleic acid may be a DNA or an RNA molecule. An exogenous nucleic acid may comprise a sequence encoding a transgene. An exogenous nucleic acid may encode a recombinant protein, such as a recombinant receptor, or a chimeric antigen receptor (CAR). An exogenous nucleic acid may be referred to as a “genetic cargo” in the context of the exogenous nucleic acid being delivered inside a cell. The genetic cargo may be a DNA or an RNA. Genetic material can generally be delivered inside a cell ex vivo by a few different known techniques using either chemical (CaCl₂—medicated transfection), or physical (electroporation), or biological (e.g. viral infection or transduction) means.

Provided herein are compositions and methods for stable, non-viral transfer and integration of genetic material into a cell. In one aspect, the genetic material is a self-integrating polynucleotide. The genetic material can be stably integrated in the genome of the cell. The cell may be a human cell. The method is designed for a safe and reliable integration of a genetic material into the genome of a cell.

Provided herein is pharmaceutical composition comprising a therapeutically effective amount of one or more polynucleic acids, or at least one vector encoding the one or more polynucleic acids, the one or more polynucleic acids comprising: (a) a mobile genetic element comprising a sequence encoding a polypeptide; and (b) an insert sequence, wherein the insert sequence comprises a sequence that is a reverse complement of a sequence encoding an exogenous therapeutic polypeptide, wherein the polypeptide encoded by the sequence of the mobile genetic element promotes integration of the insert sequence into a genome of a cell; and wherein the pharmaceutical composition is substantially non-immunogenic to a human subject.

In some embodiments, the polypeptide encoded by the sequence of the mobile genetic element comprises one or more long interspersed nuclear element (LINE) polypeptides, wherein the one or more LINE polypeptides comprises: (i) human ORF1p or a functional fragment thereof, and (ii) human ORF2p or a functional fragment thereof.

In some embodiments, the insert sequence stably integrates and/or is retrotransposed into the genome of a human cell.

In some embodiments, the human cell is an immune cell selected from the group consisting of a T cell, a B cell, a myeloid cell, a monocyte, a macrophage and a dendritic cell.

In some embodiments, the insert sequence is integrated into the genome (i) by cleavage of a DNA strand of a target site by an endonuclease encoded by the one or more polynucleic acids, (ii) via target-primed reverse transcription (TPRT) or (iii) via reverse splicing of the insert sequence into a DNA target site of the genome.

In some embodiments, the insert sequence is integrated into the genome at a poly T site using specificity of an endonuclease domain of the human ORF2p.

In some embodiments, the poly T site comprises the sequence TTTTTA.

In some embodiments, the one or more polynucleic acids comprises homology arms complementary to a target site in the genome.

In some embodiments, the insert sequence integrates into: (a) the genome at a locus that is not a ribosomal locus; (b) a gene or regulatory region of a gene of the genome, thereby disrupting the gene or downregulating expression of the gene; (c) a gene or regulatory region of a gene of the genome, thereby upregulating expression of the gene; or (d) the genome and replaces a gene of the genome.

In some embodiments, the pharmaceutical composition further comprises (i) one or more siRNAs and/or (ii) an RNA guide sequence or a polynucleic acid encoding the RNA guide sequence, and wherein the RNA guide sequence targets a DNA target site of the genome and the insert sequence is integrated into the genome at the DNA target site of the genome.

In some embodiments, one or more genes are knocked down in the methods provided herein. In some embodiments, one or more siRNAs are employed in the compositions or methods described herein. For example, one or more genes can be knocked down to enhance integration, such as through modulating a pathway that may inhibit LINE-1. In some embodiments, the one or more genes knocked down include ADAR1, ADAR2 (ADAR1B), APOBEC3C, BRCA1, let-7 miRNA, RNase L, TASHOR (HUSH complex) and/or RAD51. For example, knock down of RNase L can be used to enhance integration by inhibiting or preventing degradation of an mRNA, such as an mRNA transcribed from a LINE-1. For example, knock down of ADAR1, ADAR2 (ADAR1B), and/or BRCA1 can be used to enhance integration by inhibiting or preventing ADAR1, ADAR2 (ADAR1B), and/or BRCA1 from inhibiting the cis binding of ORF2p to a poly A tail for L1 RNP assembly. For example, knock down of let-7 miRNA can be used to enhance integration by inhibiting or preventing let-7 miRNA from inhibiting translation, such as translation of ORF2p. let-7 miRNA. For example, knock down of RAD51 and/or BRCA1 can be used to enhance integration by inhibiting or preventing repair of cleaved DNA by RAD51 and/or BRCA1.

In some embodiments, the one or more polynucleic acids have a total length of from 3 kb to 20 kb.

In some embodiments, the one or more polynucleic acids comprises one or more polyribonucleic acids, one or more RNAs or one or more mRNAs.

In some embodiments, the exogenous therapeutic polypeptide is selected from the group consisting of a ligand, an antibody, a receptor, an enzyme, a transport protein, a structural protein, a hormone, a contractile protein, a storage protein and a transcription factor.

In some embodiments, the exogenous therapeutic polypeptide is a receptor selected from the group consisting of a chimeric antigen receptor (CAR) and a T cell receptor (TCR).

In some embodiments, the one or more polynucleic acids comprises a first expression cassette comprising a promoter sequence, a 5′ UTR sequence, a 3′ UTR sequence and a poly A sequence; wherein: (i) the promoter sequence is upstream of the 5′ UTR sequence, (ii) the 5′ UTR sequence is upstream of the sequence of the mobile genetic element encoding a polypeptide, (iii) the 3′ UTR sequence is downstream of the insert sequence; and (iv) the 3′ UTR is upstream of the poly A sequence; and wherein the 5′ UTR sequence, the 3′ UTR sequence or the poly A sequence comprises a binding site for a human ORF2p or a functional fragment thereof.

In some embodiments, the insert sequence comprises a second expression cassette comprising a sequence that is a reverse complement of a promoter sequence, a sequence that is a reverse complement of a 5′ UTR sequence, a sequence that is a reverse complement of a 3′ UTR sequence and a sequence that is a reverse complement of a poly A sequence; wherein: (i) the sequence that is a reverse complement of a promoter sequence is downstream of the sequence that is a reverse complement of a 5′ UTR sequence, (ii) the sequence that is a reverse complement of a 5′ UTR sequence is downstream of the sequence that is a reverse complement of a sequence encoding an exogenous therapeutic polypeptide (iii) the sequence that is a reverse complement of a 3′ UTR sequence is upstream of the sequence that is a reverse complement of a sequence encoding an exogenous therapeutic polypeptide, and (iv) the sequence that is a reverse complement of a poly A sequence is upstream of the sequence that is a reverse complement of a 3′ UTR sequence and downstream of the sequence of the mobile genetic encoding a polypeptide.

In some embodiments, the promoter sequence of the first expression cassette is different from the promoter sequence of the second expression cassette.

In some embodiments, the one or more LINE polypeptides comprises a first LINE polypeptide comprising the human ORF1p or functional fragment thereof and a second LINE polypeptide comprising the human ORF2p or functional fragment thereof, wherein the first LINE polypeptide and the second LINE polypeptide are translated from different open reading frames (ORFs).

In some embodiments, the one or more polynucleic acids comprises a first polynucleic acid molecule encoding the human ORF1p or functional fragment thereof and a second polynucleic acid molecule encoding the human ORF2p or functional fragment thereof.

In some embodiments, the one or more polynucleic acids comprises a 5′ UTR sequence and a 3′ UTR sequence, wherein (a) the 5′ UTR comprises a 5′ UTR from LINE-1 or a sequence with at least 80% sequence identity to ACUCCUCCCCAUCCUCUCCCUCUGUCCCUCUGUCCCUCUGACCCUGCACUGUCCCAGCACC (SEQ ID NO: 51); and/or (b) the 3′ UTR comprises a 3′ UTR from LINE-1 or a sequence with at least 80% sequence identity to

(SEQ ID NO: 52)
CAGGACACAGCCUUGGAUCAGGACAGAGACUUGGG
GGCCAUCCUGCCCCUCCAACCCGACAUGUGUACCU
CAGCUUUUUCCCUCACUUGCAUCAAUAAAGCUUCU
GUGUUUGGAACAG.

In some embodiments, the sequence encoding the exogenous therapeutic polypeptide does not comprise introns.

In some embodiments, the polypeptide encoded by the sequence of the mobile genetic element comprises a C-terminal nuclear localization signal (NLS), an N-terminal NLS or both.

In some embodiments, the sequence encoding the exogenous polypeptide is not in frame with a sequence encoding the ORF1p or functional fragment thereof and/or is not in frame with a sequence encoding the ORF2p or functional fragment thereof.

In some embodiments, the one or more polynucleic acids comprises a sequence encoding a nuclease domain, a nuclease domain that is not derived from ORF2p, a megaTAL nuclease domain, a TALEN domain, a Cas9 domain, a Cas6 domain, a Cas7 domain, a Cas8 domain, a zinc finger binding domain from an R2 retroelement, or a DNA binding domain that binds to repeat sequences.

In some embodiments, the one or more polynucleic acids comprises a sequence encoding the nuclease domain, wherein the nuclease domain does not have nuclease activity or comprises a mutation that reduces activity of the nuclease domain compared to the nuclease domain without the mutation.

In some embodiments, the ORF2p or functional fragment thereof lacks endonuclease activity or comprises a mutation selected from the group consisting of S228P and Y1 180A, and/or wherein the ORF1p or functional fragment comprises a K3R mutation.

In some embodiments, the insert sequence comprises a sequence that is a reverse complement of a sequence encoding two or more exogenous therapeutic polypeptides.

In some embodiments, the one or more polynucleic acids comprises one or more polyribonucleic acids, wherein the exogenous therapeutic polypeptide is a receptor selected from the group consisting of a chimeric antigen receptor (CAR) and a T cell receptor (TCR), and wherein the pharmaceutical composition is formulated for systemic administration to a human subject.

In some embodiments, the one or more polynucleic acids (i) are formulated in a nanoparticle selected from the group consisting of a lipid nanoparticle and a polymeric nanoparticle; and/or (ii) comprises one or more polynucleic acids selected from the group consisting of glycosylated RNAs, circular RNAs and self-replicating RNAs.

Also provided herein is a method of treating a disease or condition in a human subject in need thereof comprising administering a pharmaceutical composition described herein to the human subject.

Also provided herein is a method of modifying a population of human cells ex vivo comprising contacting a composition to a population of human cell ex vivo, thereby forming an ex vivo modified population of human cells, the composition comprising one or more polynucleic acids, or at least one vector encoding the one or more polynucleic acids, the one or more polynucleic acids comprising: (a) a mobile genetic element comprising a sequence encoding a polypeptide; and (b) an insert sequence, wherein the insert sequence is a reverse complement of a sequence encoding an exogenous therapeutic polypeptide, wherein the ex vivo modified population of human cells is substantially non-immunogenic to a human subject.

In one aspect, provided herein are compositions and methods that allow integration of genetic material into the genome of a cell, wherein the genetic material that can be integrated is not specifically restricted by size. In some aspects, the method described herein provides a one-step, single polynucleotide-mediated delivery and integration of genetic “cargo” in the genome of a cell. The genetic material may comprise a coding sequence, e.g., a sequence encoding a transgene, a peptide, a recombinant protein, or an antibody or fragments thereof, wherein the method and compositions ensure stable expression of the transcribed product encoded by the coding sequence. The genetic material may comprise a non-coding sequence, for example, a regulatory RNA sequences, e.g., a regulatory small inhibitory RNA (siRNA), microRNA (miRNA), long non-coding RNA (lncRNA), or one or more transcription regulators such as a promoter and/or an enhancer, and may also include, but not limited to structural biomolecules such as ribosomal RNA (rRNA), transfer RNA (tRNA) or a fragment thereof or a combination thereof.

In another aspect, provided herein are methods and compositions for site-specific integration of a genetic material that may not be specifically restricted by size, into the genome of a cell via a non-viral delivery that ensures both safety and efficacy of the transfer. Provided methods and compositions may be particularly useful in developing a therapeutic, such as a therapeutic comprising a polynucleotide comprising a genetic material and a machinery that allows transfer into a cell and stable integration into the genome of the cell into which the polynucleotide or an mRNA encoding the polynucleotide is transferred. In some embodiments, the therapeutic may be a cell that comprises a polynucleotide that has been stably integrated into the genome of the cell using the methods and compositions described herein.

In one aspect, the present disclosure provides compositions and methods for stable gene transfer into a cell. In some embodiments, the compositions and methods are for stable gene transfer into an immune cell. In some cases, the immune cell is a myeloid cell. In some cases, the methods described herein relate to development of myeloid cells for immunotherapy.

Provided herein is a method of treating a disease in a subject in need thereof, comprising: administering a pharmaceutical composition to the subject wherein the pharmaceutical composition comprises a polycistronic mRNA sequence encoding a gene or fragment thereof, operably linked to a sequence encoding an L1 retrotransposon; wherein the gene or the fragment thereof is at least 10.1 kb in length.

Provided herein is a method for integrating a nucleic acid sequence into the genome of a cell, comprising contacting the cell with a composition comprising a polycistronic mRNA sequence encoding a gene or fragment thereof, operably linked to a sequence encoding an L1 retrotransposon; wherein the gene or the fragment thereof is at least 10.1 kb in length. In some embodiments, the gene or the fragment thereof (e.g., the payload) is at least about 10.2 kb, 10.3kb, 10.4kb, 10.5 kb, 10.6 kb, 10.7 kb, 10.8 kb, 10.9 kb, 11kb, 12 kb, 13kb, 14kb, 15kb, 16kb, 17kb, 18kb, 19kb, 20 kb or more in length.

Provided herein is a method for integrating a nucleic acid sequence into the genome of a cell, comprising contacting the cell with a composition comprising a polycistronic mRNA sequence encoding a gene or fragment thereof, operably linked to a sequence encoding an L1 retrotransposon; wherein the gene or the fragment thereof is selected from a group consisting of ABCA4, MY07A, CEP290, CDH23, EYS, USH2a, GPR98, ALMS1, GDE, OTOF, and F8.

Provided herein is a method of expressing a protein encoded by a recombinant nucleic acid in a cell, the method comprising integrating a nucleic acid sequence into the genome of a cell by contacting the cell with a composition comprising a polycistronic mRNA sequence encoding a gene or fragment thereof, operably linked to a sequence encoding an L1 retrotransposon; and expressing a protein encoded by the gene or fragment thereof, wherein expression of the protein is detectable more than 30 days after (a).

In one embodiment of a method described herein, the disease is a genetic disease.

Provided herein is a method of treating Stargardt disease, LCA10, USH1D, DFNB12, retinitis pigmentosa (RP) USH2A, USH2C, Alstrom syndrome, Glycogen storage disease III, Non-syndromic deafness, Hemophilia A, or Leber congenital amaurosis in a subject, the method comprising: (i) introducing into the subject an mRNA encoding a suitable gene or a fragment thereof, operably linked to a human L1 transposon, or (ii) introducing to the subject a population of cells comprising an mRNA encoding a suitable gene or a fragment thereof, operably linked to a human L1 transposon.

In one embodiment of a method described herein, the method comprises treating Stargardt disease in a subject in need thereof, and wherein the mRNA encodes an ABCA4 gene, or a fragment thereof.

In one embodiment of a method described herein, the method comprises treating Usher Syndrome Type 1b (Usher 1b) disease in a subject in need thereof, and wherein the mRNA encodes an MY07A gene, or a fragment thereof.

In one embodiment of a method described herein, the method comprises treating Leber congenital amaurosis (LCA)10 disease in a subject in need thereof, and wherein the mRNA encodes a CEP290 gene, or a fragment thereof.

In one embodiment of a method described herein, the method comprises treating a User Syndrome Type 1D (USH1D) non-syndromic deafness or hearing loss USH1D, DFN12 disease in a subject in need thereof, and wherein the mRNA encodes a CDH23 gene, or a fragment thereof.

In one embodiment of a method described herein, the method comprises treating a retinitis pigmentosa (RP) disease in a subject in need thereof, and wherein the mRNA encodes an EYS gene, or a fragment thereof.

In one embodiment of a method described herein, the method comprises treating a User Syndrome Type 2A (USH2A) and wherein the mRNA encodes an USH2a gene, or a fragment thereof.

In one embodiment of a method described herein, the method comprises treating a User Syndrome Type 2C (USH2C) and wherein the mRNA encodes a GPR98 gene, or a fragment thereof.

In one embodiment of a method described herein, the method comprises treating an Alstrom Syndrome, and wherein the mRNA encodes an ALMS1 gene, or a fragment thereof.

In one embodiment of a method described herein, the method comprises treating a Glycogen Storage Disease III, and wherein the mRNA encodes a GDE gene, or a fragment thereof.

In one embodiment of a method described herein, the method comprises treating a non-syndromic deafness or hearing loss and wherein the mRNA encodes an OTOF gene, or a fragment thereof.

In one embodiment of a method described herein, the method comprises treating Hemophilia A, and the mRNA encodes an Factor VIII (F8) gene, or a fragment thereof.

Provided herein is a method for targeted replacement of a genomic nucleic acid sequence of a cell, the method comprising: (A) introducing to the cell a polynucleotide sequence encoding a first protein complex comprising a targeted excision machinery for excising from the genome of the cell a nucleic acid sequence comprising one or more mutations; and (B) a recombinant mRNA encoding a second protein complex, wherein the recombinant mRNA comprises: (i) a nucleic acid sequence comprising the excised nucleic acid sequence in (A) that does not contain the one or more mutations, and (ii) a sequence encoding an L1 retrotransposon ORF2 protein under the influence of an independent promoter.

In one embodiment of a method described herein, the nucleic acid sequence comprising the one or more mutations comprises a pathogenic variant of a cellular gene.

In one embodiment of a method described herein, the a nucleic acid sequence in (B) comprising the nucleic acid sequence that does not contain the one or more mutations is operably linked to the ORF2 sequence.

In one embodiment of a method described herein, the method further comprising introducing a sequence comprising a plurality of thymidine residues at the excision site.

In some embodiment, introducing the sequence comprises introducing at least four thymidine residues.

In one embodiment of a method described herein, the targeted excision machinery comprises a sequence guided site-specific excision endonuclease.

In one embodiment of a method described herein, the targeted excision machinery comprises a CRISPR-CAS system.

In some embodiments, the targeted excision machinery is a modified recombinant LINE 1 (L1) endonuclease.

In some embodiments, introducing the sequence comprising a plurality of thymidine residues comprises base extension by prime editing at the excision site.

In some embodiments, the mRNA sequence encoding an L1 retrotransposon ORF2 protein further comprises a sequence encoding the L1 retrotransposon ORF1 protein.

In some embodiments, the mRNA comprises a sequence for an inducible promoter.

In one embodiment of a method described herein, the excised sequence is greater than 1000 bases.

In one embodiment of a method described herein, the excised sequence is greater than 6kb.

In one embodiment of a method described herein, the excised sequence is about 10kb.

In some embodiments, the cell is a lymphocyte. In some embodiments, the cell is a myeloid cell. In some embodiments, the cell is an epithelial cell. In some embodiments, the cell is a cancer cell.

In some embodiments, the nucleic acid sequence encodes an ATP-binding cassette (ABC) transporter gene, (ABCA4) gene, or a fragment thereof.

In some embodiments, the nucleic acid sequence encodes an MY07A, CEP290, CDH23, EYS, USH2a, GPR98, ALMS1, GDE, OTOF or an F8 gene or a fragment thereof.

In some embodiments, introducing comprises introducing to the cell ex vivo. In some embodiments, introducing comprises electroporation. In some embodiments, introducing comprises introducing to the cell in vivo. In some embodiments, expression of the nucleic acid sequence comprising the sequence that does not contain the one or more mutations, is detectable at least 35 days after introducing to the cell. In some embodiments, introducing into the subject comprises direct administration of the mRNA systemically.

In some embodiments, introducing into the subject comprises local administration of the mRNA.

In some embodiments, the mRNA sequence comprises a cell targeting moiety.

In some embodiments, the cell targeting moiety is an aptamer.

In some embodiments, introducing into the subject comprises introducing the mRNA in the retina of the subject.

Provided herein is a method of integrating a nucleic acid sequence into a genome of a cell, the method comprising introducing a recombinant mRNA or a vector encoding an mRNA into the cell, wherein the mRNA comprises: (a) an insert sequence, wherein the insert sequence comprises (i) an exogenous sequence, or (ii) a sequence that is a reverse complement of the exogenous sequence; (b) a 5′ UTR sequence and a 3′ UTR sequence downstream of the 5′ UTR sequence; wherein the 5′ UTR sequence or the 3′ UTR sequence comprises a binding site for a human ORF protein, and wherein the insert sequence is integrated into the genome of the cell, wherein the insert sequence is a gene selected from a group consisting of ABCA4, MY07A, CEP290, CDH23, EYS, USH2a, GPR98, ALMS1, GDE, OTOF, and F8.

In some embodiments, the 5′ UTR sequence or the 3′ UTR sequence comprises a binding site for human ORF2p.

Provided herein is a method for integrating a nucleic acid sequence into the genome of an immune cell, the method comprising introducing a recombinant mRNA or a vector encoding an mRNA, wherein the mRNA comprises: (a) an insert sequence, wherein the insert sequence comprises (i) an exogenous sequence or (ii) a sequence that is a reverse complement of the exogenous sequence; (b) 5′ UTR sequence and a 3′ UTR sequence downstream of the 5′ UTR sequence, wherein the 5′ UTR sequence or the 3′ UTR sequence comprises an endonuclease binding site and/or a reverse transcriptase binding site, and wherein the insert sequence is integrated into the genome of the immune cell, wherein the insert sequence is a gene selected from a group consisting of ABCA4, MY07A, CEP290, CDH23, EYS, USH2a, GPR98, ALMS1, GDE, OTOF, and F8.

Provided herein is a method for integrating a nucleic acid sequence into the genome of a cell, the method comprising introducing a recombinant mRNA or a vector encoding an mRNA, wherein the mRNA comprises: (a) an insert sequence, wherein the insert sequence comprises (i) an exogenous sequence or (ii) a sequence that is a reverse complement of the exogenous sequence; (b) a 5′ UTR sequence, a sequence of a human retrotransposon downstream of the 5′ UTR sequence, and a 3′ UTR sequence downstream of the sequence of a human retrotransposon; wherein the 5′ UTR sequence or the 3′ UTR sequence comprises an endonuclease binding site and/or a reverse transcriptase binding site, and wherein the sequence of a human retrotransposon encodes for two proteins that are translated from a single RNA containing two ORFs, and wherein the insert sequence is integrated into the genome of the cell, wherein the insert sequence is a gene selected from a group consisting of ABCA4, MY07A, CEP290, CDH23, EYS, USH2a, GPR98, ALMS1, GDE, OTOF, and F8.

In some embodiments, the 5′ UTR sequence or the 3′ UTR sequence comprises an ORF2p binding site. In some embodiments, the ORF2p binding site is a poly A sequence in the 3′ UTR sequence.

In some embodiments, the mRNA comprises a sequence of a human retrotransposon. In some embodiments, the sequence of a human retrotransposon is downstream of the 5′ UTR sequence.

In some embodiments, the sequence of a human retrotransposon is upstream of the 3′ UTR sequence. In some embodiments, the sequence of a human retrotransposon encodes for two proteins that are translated from a single RNA containing two ORFs. In some embodiments, the two ORFs are non-overlapping ORFs.

In some embodiments, the sequence of a human retrotransposon comprises a sequence of a non-LTR retrotransposon. In some embodiments, the sequence of a human retrotransposon encodes comprises a LINE-1 retrotransposon. In some embodiments, the LINE-1 retrotransposon is a human LINE-1 retrotransposon. In some embodiments, the sequence of a human retrotransposon comprises a sequence encoding an endonuclease and/or a reverse transcriptase.

In some embodiments, the endonuclease and/or a reverse transcriptase is ORF2p.

In some embodiments, the reverse transcriptase is a group II intron reverse transcriptase domain.

In some embodiments, the endonuclease and/or a reverse transcriptase is a minke whale endonuclease and/or a reverse transcriptase.

In some embodiments, the sequence of a human retrotransposon comprises a sequence encoding ORF2p. In some embodiments, the insert sequence is integrated into the genome at a poly T site using specificity of an endonuclease domain of the ORF2p. In some embodiments, the poly T site comprises the sequence TTTTTA. In some embodiments, the retrotransposon comprises an ORF1p and/or the ORF2p fused to a nuclear retention sequence. In some embodiments, the nuclear retention sequence is an Alu sequence. In some embodiments, the ORF1p and/or the ORF2p is fused to an MS2 coat protein. In some embodiments, the 5′ UTR sequence or the 3′ UTR sequence comprises at least one, two, three or more MS2 hairpin sequences.

Provided herein is a composition comprising a recombinant mRNA or vector encoding an mRNA, wherein the mRNA comprises a human LINE-1 transposon sequence comprising: (i) a human LINE-1 transposon 5′ UTR sequence, (ii) a sequence encoding ORF1p downstream of the human LINE-1 transposon 5′ UTR sequence, (iii) an inter-ORF linker sequence downstream of the sequence encoding ORF1p, (iv) a sequence encoding ORF2p downstream of the inter-ORF linker sequence, and (v) a 3′ UTR sequence derived from a human LINE-1 transposon downstream of the sequence encoding ORF2p; wherein the 3′ UTR sequence comprises an insert sequence, wherein the insert sequence is a reverse complement of a sequence encoding an exogenous polypeptide or a reverse complement of a sequence encoding an exogenous regulatory element, wherein the insert sequence is a gene selected from a group consisting of ABCA4, MY07A, CEP290, CDH23, EYS, USH2a, GPR98, ALMS1, GDE, OTOF, and F8.

Provided herein is a composition comprising a nucleic acid comprising a nucleotide sequence encoding (a) a long interspersed nuclear element (LINE) polypeptide, wherein the LINE polypeptide includes human ORF1p and human ORF2p; and (b) an insert sequence, wherein the insert sequence is a reverse complement of a sequence encoding an exogenous polypeptide or a reverse complement of a sequence encoding an exogenous regulatory element, wherein the composition is substantially non-immunogenic, wherein the insert sequence is a gene selected from a group consisting of ABCA4, MY07A, CEP290, CDH23, EYS, USH2a, GPR98, ALMS1, GDE, OTOF, and F8.

Immunotherapy using phagocytic cells involves making and using engineered myeloid cells, such as macrophages or other phagocytic cells that attack and kill diseased cells, such as cancer cells, or infected cells. Engineered myeloid cells, such as macrophages and other phagocytic cells are prepared by incorporating in them via recombinant nucleic acid technology, a synthetic, recombinant nucleic acid encoding an engineered protein, such as a chimeric antigen receptor, that comprises a targeted antigen binding extracellular domain that is designed to bind to specific antigens on the surface of a target, such as a target cell, such as a cancer cell. Binding of the engineered chimeric receptor to an antigen on a target, such as cancer antigen (or likewise, a disease target), initiates phagocytosis of the target. This triggers two fold action: one, phagocytic engulfment and lysis of the target destroys the target and eliminates it as a first line of immune defense; two, antigens from the target are digested in the phagolysosome of the myeloid cell, are presented on the surface of the myeloid cell, which then leads to activation of T cells and further activation of the immune response and development of immunological memory. Chimeric receptors are engineered for enhanced phagocytosis and immune activation of the myeloid cell in which it is incorporated and expressed. Chimeric antigen receptors of the disclosure are variously termed herein as a chimeric fusion protein, CFP, phagocytic receptor (PR) fusion protein (PFP), or chimeric antigen receptor for phagocytosis (CAR-P), while each term is directed to the concept of a recombinant chimeric and/or fusion receptor protein. In some embodiments, genes encoding non-receptor proteins are also co-expressed in the myeloid cells, typically for an augmentation of the chimeric antigen receptor function. In summary, contemplated herein are various engineered receptor and non-receptor recombinant proteins that are designed to augment phagocytosis and or immune response of a myeloid cell against a disease target, and methods and compositions for creating and incorporating recombinant nucleic acids that encode the engineered receptors or non-receptor recombinant protein, such that the methods and compositions are suitable for creating an engineered myeloid cell for immunotherapy.

In one aspect, the present disclosure provides compositions and methods for stable gene transfer into a cell, where the cell can be any somatic cell. In some embodiments the compositions and methods are designed for cell-specific or tissue-specific delivery. In some cases, the methods described herein relate to supplying a functional protein or a fragment thereof to compensate for an absent or defective (mutated) protein in vivo, e.g., for a protein replacement therapy.

Incorporation of a recombinant nucleic acid in a cell can be accomplished by one or more gene transfer techniques that are available in the state of the art. However, incorporation of exogenous genetic (e.g., nucleic acid) elements into the genome for therapeutic purposes still faces several challenges.

Achieving stable integration in a safe and dependable manner, and efficient and prolonged expression are a few among them. Most of the successful gene transfer systems aimed at genomic integration of the cargo nucleic acid sequence rely on viral delivery mechanisms, which have some inherent safety and efficacy issues. Delivery and integration of long nucleic acid sequences cannot be achieved by current gene editing systems.

Little attention has so far been devoted to making and using engineered myeloid cells for stable long-term gene transfer and expression of the transgene. For example, gene transfer to differentiated mammalian cells ex vivo for cell therapy can be accomplished via viral gene transfer mechanisms.

However, there are several strategic disadvantages associated with the use of viral gene-transfer vectors, including an undesired potential for transgene silencing over time, the preferential integration into transcriptionally active sites of the genome with associated undesired activation of other genes (e.g. oncogenes) and genotoxicity. In addition to the safety issues increased expense and cumbersome effort of manufacturing, storing and handling integrating viruses often stand in the way of large-scale use of viral vector mediated of gene-modified cells in therapeutic applications. These persistent concerns associated with viral vectors regarding safety, as well as cost and scale of vector production necessitates alternative methods for effective therapy.

Integration of a transgene into the genome of a cell to be used for an immunotherapy can be advantageous in the sense that it is stable and a lower number of cells is required for delivery during the therapy. On the other hand, integrating a transgene in a non-dividing cell can be challenging in both affecting the health and function of the cell as well as the ultimate lifespan of the cell in vivo, and therefore affects its overall utility as the therapeutic. In some embodiments, the methods described herein for generating a myeloid cell for immunotherapy can be a cumulative product of a number of steps and compositions involving but not limited to, for example, selecting a myeloid cell for modifying; method and compositions for incorporating a recombinant nucleic acid in a myeloid cell; methods and compositions for enhancing expression of the recombinant nucleic acid; methods and compositions for selecting and modifying vectors; methods of preparing a recombinant nucleic acid suitable for in vivo administration for uptake and incorporation of the recombinant nucleic acid by a myeloid cell in vivo and therefore generating a myeloid cell for therapy. In some aspects, one or more embodiments of the various inventions described herein are transferrable among each other, and one of skill in the art is expected to use them in alternatives, combinations or interchangeably without the necessity of undue experimentation. All such variations of the disclosed elements are contemplated and fully encompassed herein.

In one aspect, transposons, or transposable elements (TEs) are considered herein, for means of incorporating a heterologous, synthetic or recombinant nucleic acid encoding a transgene of interest in a myeloid cell. Transposon, or transposable elements are genetic elements that have the capability to transpose fragments of genetic material into the genome by use of an enzyme known as transposase.

Mammalian genomes contain a high number of transposable element (TE)-derived sequences, and up to 70% of our genome represents TE-derived sequences (de Koning et al. 2011; Richardson et al. 2015).

These elements could be exploited to introduce genetic material into the genome of a cell. The TE elements are capable of mobilization, often termed as “jumping” genetic material within the genome. TEs generally exist in eukaryotic genomes in a reversibly inactive, epigenetically silenced form. In the present disclosure methods and compositions for efficient and stable integration of transgenes into macrophages and other phagocytic cells. The method is based on use of a transposase and transposable elements mRNA-encoded transposase. In some embodiments, Long Interspersed Element-1 (L 1) RNAs are used for stable integration and/or retrotransposition of the transgene into a cell (e.g., a macrophage or phagocytic cell.

Contemplated herein are methods for retrotransposon mediated stable integration of an exogenous nucleic acid sequence into the genome of a cell. The method may take advantage of the random genomic integration machinery of the retrotransposon into the cell without creating an adverse effect. Methods described herein can be used for robust and versatile incorporation of an exogenous nucleic acid sequence into a cell, such that the exogenous nucleic acid is incorporated at a safe locus within the genome and is expressed without being silenced by the cell's inherent defense mechanism. The method described herein can be used to incorporate an exogenous nucleic acid that is about 1 kb, about 2 kb, about 3 kb, about 4 kb, about 5 kb, about 6 kb, about 7kb about 8 kb, about 9 kb, about 10 kb, or more in size. In some embodiments, the exogenous nucleic acid is not incorporated within a ribosomal locus. In some embodiments, the exogenous nucleic acid is not incorporated within a ROSA26 locus, or another safe harbor locus. In some embodiments, the methods and compositions described herein can incorporate an exogenous nucleic acid sequence anywhere within the genome of the cell. Furthermore, contemplated herein is a retrotransposition system that is developed to incorporate an exogenous nucleic acid sequence into a specific predetermined site within the genome of a cell, without creating an adverse effect. The disclosed methods and compositions incorporate several mechanisms of engineering the retrotransposons for highly specific incorporation of the exogenous nucleic acid into a cell with high fidelity. Retrotransposons chosen for this purpose may be a human retrotransposon.

Methods and compositions described herein represent a salient breakthrough in the molecular systems and mechanisms for manipulating the genome of a cell. Shown here for the first time is a method that exploits a human retrotransposon system into non-virally delivering and stably integrating a large fragment of exogenous nucleic acid sequence (at least greater than 100 nucleobases, at least greater than 1 kb, at least greater than 2 kb, at least greater than 3 kb, etc.) into a non-conserved region of the genome that is not an rDNA or a ribosomal locus or a designated safe-harbor locus such as the ROSA 26 locus.

In some embodiments, a retrotransposable system is used to stably incorporate into the genome and express a non-endogenous nucleic acid, where the non-endogenous nucleic acid comprises retrotransposable elements within the nucleic acid sequence. In some embodiments, a cell's endogenous retrotransposable system (e.g., proteins and enzymes) is used to stably express a non-endogenous nucleic acid in the cell. In some embodiments, a cell's endogenous retrotransposable system (e.g., proteins and enzymes, such as a LINE-1 retrotransposition system) is used, but may further express one or more components of the retrotransposable system to stably express a non-endogenous nucleic acid in the cell.

In some embodiments, a synthetic nucleic acid is provided herein, the synthetic nucleic acid encoding a transgene, and encoding one or more components for genomic integration and/or retrotransposition.

In one aspect, provided herein is a method of integrating a nucleic acid sequence into a genome of a cell, the method comprising introducing a recombinant mRNA or a vector encoding an mRNA into the cell, wherein the mRNA comprises: an insert sequence, wherein the insert sequence comprises an exogenous sequence, or a sequence that is a reverse complement of the exogenous sequence; a 5′ UTR sequence and a 3′ UTR sequence downstream of the 5′ UTR sequence; wherein the 5′ UTR sequence or the 3′ UTR sequence comprises a binding site for a human ORF protein, and wherein the insert sequence is integrated into the genome of the cell. In some embodiments, the 5′ UTR sequence or the 3′ UTR sequence comprises a binding site for human ORF2p.

In one aspect, provided herein is a method for integrating a nucleic acid sequence into the genome of an immune cell, the method comprising introducing a recombinant mRNA or a vector encoding an mRNA, wherein the mRNA comprises an insert sequence, wherein the insert sequence comprises (i) an exogenous sequence or (ii) a sequence that is a reverse complement of the exogenous sequence; 5′ UTR sequence and a 3′ UTR sequence downstream of the 5′ UTR sequence, wherein the 5′ UTR sequence or the 3′ UTR sequence comprises an endonuclease binding site and/or a reverse transcriptase binding site, and wherein the transgene sequence is integrated into the genome of the immune cell.

In one aspect, provided herein is a method for integrating a nucleic acid sequence into the genome of a cell, the method comprising introducing a recombinant mRNA or a vector encoding an mRNA, wherein the mRNA comprises an insert sequence, wherein the insert sequence comprises (i) an exogenous sequence or (ii) a sequence that is a reverse complement of the exogenous sequence; a 5′ UTR sequence, a sequence of a human retrotransposon downstream of the 5′ UTR sequence, and a 3′ UTR sequence downstream of the sequence of a human retrotransposon; wherein the 5′ UTR sequence or the 3′ UTR sequence comprises an endonuclease binding site and/or a reverse transcriptase binding site, and wherein the sequence of a human retrotransposon encodes for two proteins that are translated from a single RNA containing two ORFs, and wherein the insert sequence is integrated into the genome of the cell.

In some embodiments, the 5′ UTR sequence or the 3′ UTR sequence comprises an ORF2p binding site. In some embodiments, the ORF2p binding site is a poly A sequence in the 3′ UTR sequence.

In some embodiments, the mRNA comprises a sequence of a human retrotransposon. In some embodiments, the sequence of a human retrotransposon is downstream of the 5′ UTR sequence. In some embodiments, the sequence of a human retrotransposon is upstream of the 3′ UTR sequence. In some embodiments, the polynucleotide sequence that is desired to be transferred and incorporated into the genome of a cell (e.g., the insert) is inserted at a site 3′ to the sequence encoding ORF1 in a recombinant nucleic acid construct. In some embodiments, the polynucleotide sequence that is desired to be transferred and incorporated into the genome of a cell is inserted at a site 3′ to the sequence encoding ORF2 in a recombinant nucleic acid construct. In some embodiments the sequence that is desired to be transferred and incorporated into the genome of a cell is inserted within the 3′-UTR of ORF1 or ORF2, or both. In some embodiments, the polynucleotide sequence that is sequence that is desired to be transferred and incorporated into the genome of a cell is inserted upstream of the poly A tail of ORF2 in a recombinant nucleic acid construct.

In some embodiments, the sequence of a human retrotransposon encodes for two proteins that are translated from a single RNA containing two ORFs. In some embodiments, the two ORFs are non-overlapping ORFs. In some embodiments, the two ORFs are ORF1 and ORF2. In some embodiments, the ORF1 encodes ORF1p and ORF2 encodes ORF2p.

In some embodiments, the sequence of a human retrotransposon comprises a sequence of a non-LTR retrotransposon. In some embodiments, the sequence of a human retrotransposon comprises a LINE-1 retrotransposon. In some embodiments, the LINE-1 retrotransposon is a human LINE-1 retrotransposon.

In some embodiments, the sequence of a human retrotransposon comprises a sequence encoding an endonuclease and/or a reverse transcriptase. In some embodiments, the endonuclease and/or a reverse transcriptase is ORF2p. In some embodiments, the reverse transcriptase is a group II intron reverse transcriptase domain. In some embodiments, the endonuclease and/or a reverse transcriptase is a minke whale endonuclease and/or a reverse transcriptase. In some embodiments, the sequence of a human retrotransposon comprises a sequence encoding ORF2p. In some embodiments, the insert sequence is integrated into the genome at a poly T site using specificity of an endonuclease domain of the ORF2p. In some embodiments, the poly T site comprises the sequence TTTTTA.

In some embodiments, provided herein is a polynucleotide construct comprising an mRNA wherein the mRNA comprises a sequence encoding a human retrotransposon, wherein, (i) the sequence of a human retrotransposon comprises a sequence encoding ORF1p, (ii) the mRNA does not comprise a sequence encoding ORF1p, or (iii) the mRNA comprises a replacement of the sequence encoding ORF1p with a 5′ UTR sequence from the complement gene. In some embodiments, the mRNA comprises a first mRNA molecule encoding ORF1p, and a second mRNA molecule encoding an endonuclease and/or a reverse transcriptase. In some embodiments, the mRNA is an mRNA molecule comprising a first sequence encoding ORF1p, and a second sequence encoding an endonuclease and/or a reverse transcriptase. In some embodiments, the first sequence encoding ORF1p and the second sequence encoding an endonuclease and/or a reverse transcriptase are separated by a linker sequence.

In some embodiments, the linker sequence comprises an internal ribosome entry sequence (IRES). In some embodiments, the IRES is an IRES from CVB3 or EV71. In some embodiments, the linker sequence encodes a self-cleaving peptide sequence. In some embodiments, the linker sequence encodes a T2A, a E2A or a P2A sequence

In some embodiments, the sequence of a human retrotransposon comprises a sequence that encodes ORF1p fused to an additional protein sequence and/or a sequence that encodes ORF2p fused to an additional protein sequence. In some embodiments, the ORF1p and/or the ORF2p is fused to a nuclear retention sequence. In some embodiments, the nuclear retention sequence is an Alu sequence. In some embodiments, the ORF1p and/or the ORF2p is fused to an MS2 coat protein. In some embodiments, the 5′ UTR sequence or the 3′ UTR sequence comprises at least one, two, three or more MS2 hairpin sequences.

In some embodiments, the 5′ UTR sequence or the 3′ UTR sequence comprises a sequence that promotes or enhances interaction of a poly A tail of the mRNA with the endonuclease and/or a reverse transcriptase.

In some embodiments, the 5′ UTR sequence or the 3′ UTR sequence comprises a sequence that promotes or enhances interaction of a poly-A-binding proteins (e.g., PABP) with the endonuclease and/or a reverse transcriptase. In some embodiments, the 5′ UTR sequence or the 3′ UTR sequence comprises a sequence that increases specificity of the endonuclease and/or a reverse transcriptase to the mRNA relative to another mRNA expressed by the cell. In some embodiments, the 5′ UTR sequence or the 3′ UTR sequence comprises an Alu element sequence.

In some embodiments, the first sequence encoding ORF1p and the second sequence encoding an endonuclease and/or a reverse transcriptase have the same promoter. In some embodiments, the insert sequence has a promoter that is different from the promoter of the first sequence encoding ORF1p. In some embodiments, the insert sequence has a promoter that is different from the promoter of the second sequence encoding an endonuclease and/or a reverse transcriptase. In some embodiments, the first sequence encoding ORF1p and/or the second sequence encoding an endonuclease and/or a reverse transcriptase have a promoter or transcription initiation site selected from the group consisting of an inducible promoter, a CMV promoter or transcription initiation site, a T7 promoter or transcription initiation site, an EF1a promoter or transcription initiation site and combinations thereof. In some embodiments, the insert sequence has a promoter or transcription initiation site selected from the group consisting of an inducible promoter, a CMV promoter or transcription initiation site, a T7 promoter or transcription initiation site, an EF1a promoter or transcription initiation site and combinations thereof.

In some embodiments, the first sequence encoding ORF1p and the second sequence encoding an endonuclease and/or a reverse transcriptase are codon optimized for expression in a human cell.

In some embodiments, the mRNA comprises a WPRE element. In some embodiments, the mRNA comprises a selection marker. In some embodiments, the mRNA comprises a sequence encoding an affinity tag. In some embodiments, the affinity tag is linked to the sequence encoding an endonuclease and/or a reverse transcriptase.

In some embodiments, the 3′ UTR comprises a poly A sequence or wherein a poly A sequence is added to the mRNA in vitro. In some embodiments, the poly A sequence is downstream of a sequence encoding an endonuclease and/or a reverse transcriptase. In some embodiments, the insert sequence is upstream of the poly A sequence.

In some embodiments, the 3′ UTR sequence comprises the insert sequence. In some embodiments, the insert sequence comprises a sequence that is a reverse complement of the sequence encoding the exogenous polypeptide. In some embodiments, the insert sequence comprises a polyadenylation site. In some embodiments, the insert sequence comprises an SV40 polyadenylation site. In some embodiments, the insert sequence comprises a polyadenylation site upstream of the sequence that is a reverse complement of the sequence encoding the exogenous polypeptide. In some embodiments, the insert sequence is integrated into the genome at a locus that is not a ribosomal locus. In some embodiments, the insert sequence is integrated into the genome at a locus that is not a rDNA locus. In some embodiments, the insert sequence integrates into a gene or regulatory region of a gene, thereby disrupting the gene or downregulating expression of the gene. In some embodiments, the insert sequence integrates into a gene or regulatory region of a gene, thereby upregulating expression of the gene. In some embodiments, the insert sequence integrates into the genome and replaces a gene. In some embodiments, the insert sequence is stably integrated into the genome. In some embodiments, the insert sequence is retrotransposed into the genome. In some embodiments, the insert sequence is integrated into the genome by cleavage of a DNA strand of a target site by an endonuclease encoded by the mRNA. In some embodiments, the insert sequence is integrated into the genome via target-primed reverse transcription (TPRT). In some embodiments, the insert sequence is integrated into the genome via reverse splicing of the mRNA into a DNA target site of the genome.

In some embodiments, the cell is an immune cell. In some embodiments, the immune cell is a T cell or a B cell. In some embodiments, the immune cell is a myeloid cell. In some embodiments, the immune cell is selected from a group consisting of a monocyte, a macrophage, a dendritic cell, a dendritic precursor cell, and a macrophage precursor cell.

In some embodiments, the mRNA is a self-integrating mRNA. In some embodiments, the method comprises introducing into the cell the mRNA. In some embodiments, the method comprises introducing into the cell the vector encoding the mRNA. In some embodiments, the method comprises introducing the mRNA or the vector encoding the mRNA into a cell ex vivo. In some embodiments, the method further comprises administering the cell to a human subject. In some embodiments, the method comprises administering the mRNA or the vector encoding the mRNA to a human subject. In some embodiments, an immune response is not elicited in the human subject. In some embodiments, the mRNA or the vector is substantially non-immunogenic.

In some embodiments, the vector is a plasmid or a viral vector. In some embodiments, the vector comprises a non-LTR retrotransposon. In some embodiments, the vector comprises a human L1 element.

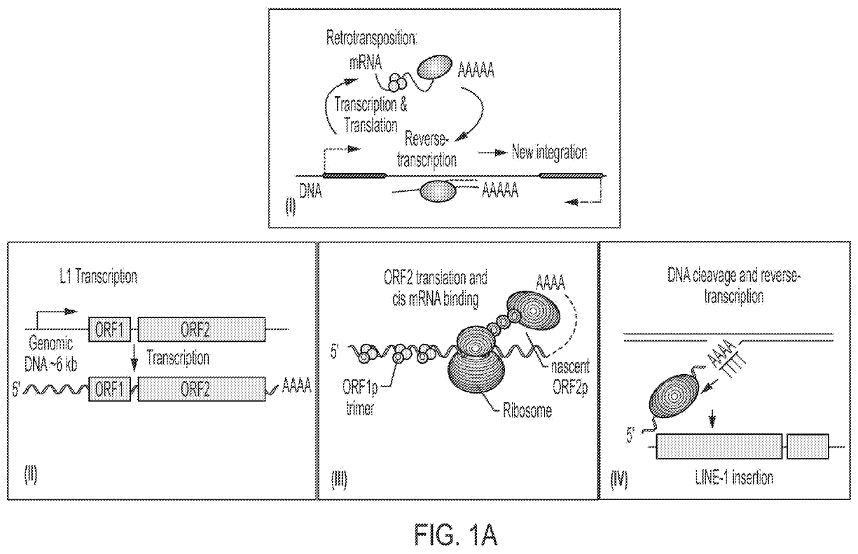

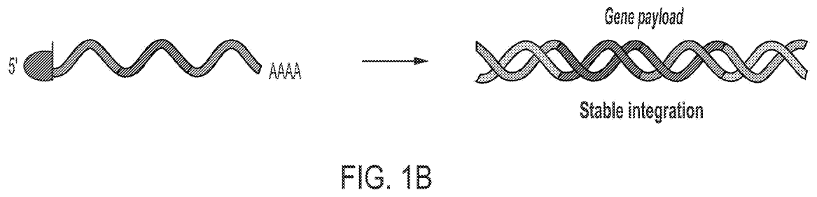

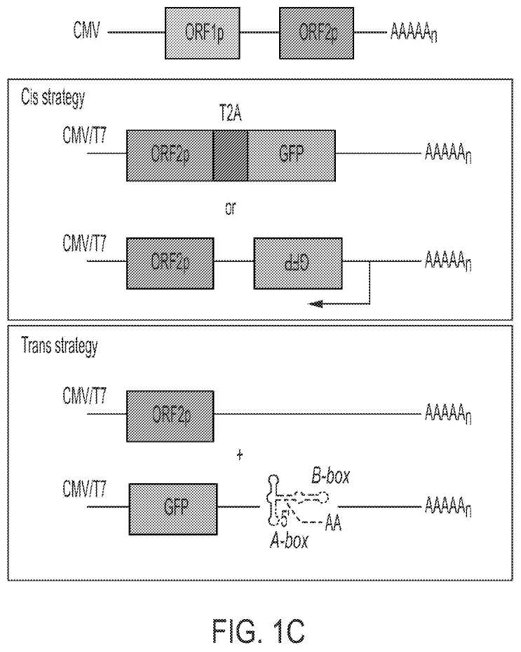

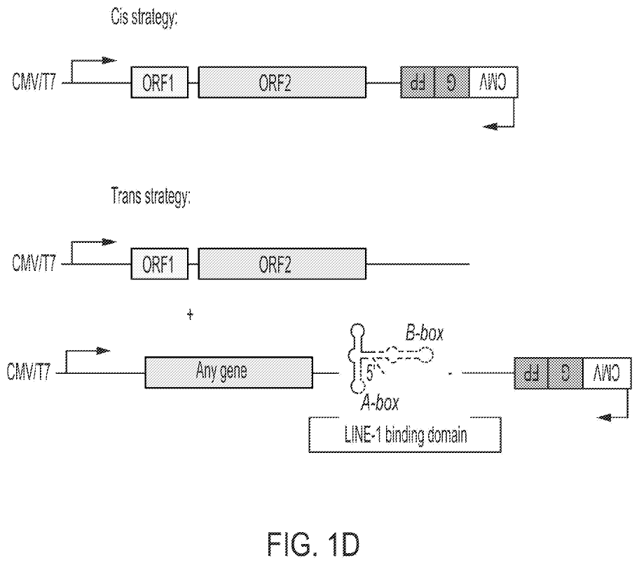

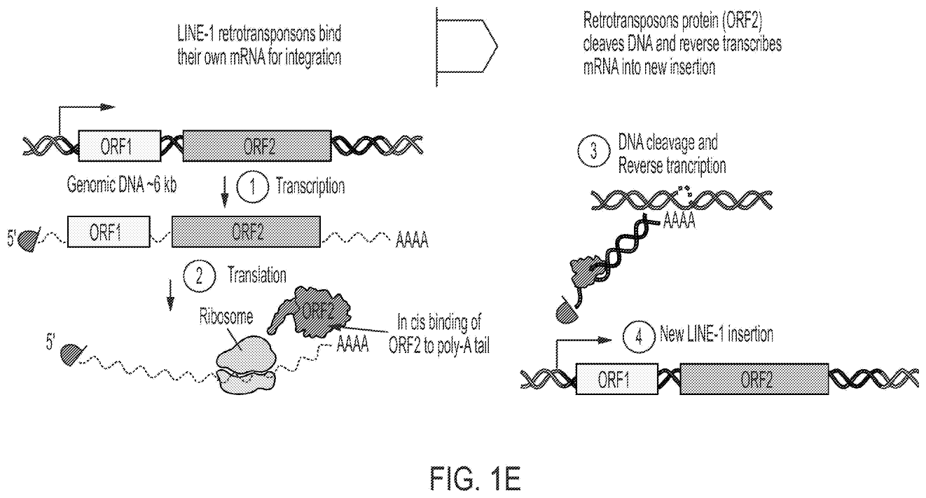

In some embodiments, the vector comprises a L1 retrotransposon ORF1 gene. In some embodiments, the vector comprises a L1 retrotransposon ORF2 gene. In some embodiments, the vector comprises a L1 retrotransposon. In some embodiments, provided herein is an mRNA comprising sequences encoding human LINE 1 retrotransposition elements, and a payload comprising a nucleic acid sequence which can be retrotransposed and integrated into a genome of a cell comprising the mRNA. In some embodiments, provided herein is an mRNA that can be delivered into a living cell, e.g., a human cell, wherein, the mRNA comprises sequences encoding human LINE 1 retrotransposition elements, and a payload comprising a nucleic acid sequence which can be retrotransposed and integrated into the genome of the cell. In some embodiments, the sequences encoding human LINE 1 retrotransposition elements comprise a L1 retrotransposon ORF1 sequence or a fragment thereof. In some embodiments, the sequences encoding human LINE 1 retrotransposition elements comprise a L1 retrotransposon ORF2 sequence or a fragment thereof. In some embodiments, the sequences encoding human LINE 1 retrotransposition elements comprise a L1 retrotransposon ORF1 sequence or a fragment thereof and a L1 retrotransposon ORF2 sequence or a fragment thereof, and a nucleic acid “payload” sequence which is a heterologous sequence which is integrated into the genome of cell by retrotransposition. (See, for example, FIG. 1B).

In some embodiments, the mRNA is at least about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 kilobases. In some embodiments, the mRNA is a most about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 kilobases. In some embodiments, the mRNA is at least about 5.1, 5.2, 5.3, 5.4, 5.5, 5.6, 5.7, 5.8, 5.9 or 6 kilobases. In some embodiments, the mRNA is at least about 6.1, 6.2, 6.3, 6.4, 6.5, 6.6, 6.7, 6.8, 6.9 or 7 kilobases. In some embodiments, the mRNA is at least about 7.1, 7.2, 7.3, 7.4, 7.5, 7.6, 7.7, 7.8, 7.9 or 8 kilobases. In some embodiments, the mRNA is at least about 8.1, 8.2, 8.3, 8.4, 8.5, 8.6, 8.7, 8.8, 8.9 or 9 kilobases. In some embodiments, the mRNA is at least about 9.1, 9.2, 9.3, 9.4, 9.5, 9.6, 9.7, 9.8, 9.9 or 10 kilobases.

In some embodiments, the mRNA comprises a sequence that inhibits or prevents degradation of the mRNA. In some embodiments, the sequence that inhibits or prevents degradation of the mRNA inhibits or prevents degradation of the mRNA by an exonuclease or an RNAse. In some embodiments, the sequence that inhibits or prevents degradation of the mRNA is a G quadruplex, pseudoknot or triplex sequence. In some embodiments, the sequence the sequence that inhibits or prevents degradation of the mRNA is an exoribonuclease-resistant RNA structure from a flavivirus RNA or an ENE element from KSV. In some embodiments, the sequence that inhibits or prevents degradation of the mRNA inhibits or prevents degradation of the mRNA by a deadenylase. In some embodiments, the sequence that inhibits or prevents degradation of the mRNA comprises non-adenosine nucleotides within or at a terminus of a poly A tail of the mRNA. In some embodiments, the sequence that inhibits or prevents degradation of the mRNA increases stability of the mRNA. In some embodiments, the exogenous sequence comprises a sequence encoding an exogenous polypeptide. In some embodiments, the sequence encoding an exogenous polypeptide is not in frame with a sequence encoding an endonuclease and/or a reverse transcriptase. In some embodiments, the sequence encoding an exogenous polypeptide is not in frame with a sequence encoding an endonuclease and/or a reverse transcriptase. In some embodiments, the exogenous sequence does not comprise introns. In some embodiments, the exogenous sequence comprises a sequence encoding an exogenous polypeptide selected from the group consisting of an enzyme, a receptor, a transport protein, a structural protein, a hormone, an antibody, a contractile protein and a storage protein. In some embodiments, the exogenous sequence comprises a sequence encoding an exogenous polypeptide selected from the group consisting of a chimeric antigen receptor (CAR), a ligand, an antibody, a receptor, and an enzyme. In some embodiments, the exogenous sequence comprises a regulatory sequence. In some embodiments, the regulatory sequence comprises a cis-acting regulatory sequence. In some embodiments, the regulatory sequence comprises a cis-acting regulatory sequence selected from the group consisting of an enhancer, a silencer, a promoter or a response element. In some embodiments, the regulatory sequence comprises a trans-acting regulatory sequence. In some embodiments, the regulatory sequence comprises a trans-acting regulatory sequence that encodes a transcription factor.

In some embodiments, integration of the insert sequence does not adversely affect cell health. In some embodiments, the endonuclease, the reverse transcriptase or both are capable of site-specific integration of the insert sequence.

In some embodiments, the retrotransposon system used herein is further engineered for precise site-specific integration. In some embodiments the retrotransposon system used herein is paired with a CRISPR-Cas system to increase specificity. In some embodiments, the ORF polypeptide-binding sequence, e.g., TTTTTA may be engineered site-specifically into a genomic sequence of a cell.

In some embodiments, the mRNA comprises a sequence encoding an additional nuclease domain or a nuclease domain that is not derived from ORF2. In some embodiments, the mRNA comprises a sequence encoding a megaTAL nuclease domain, a TALEN domain, a Cas9 domain, a zinc finger binding domain from an R2 retroelement, or a DNA binding domain that binds to repetitive sequences such as a Rep78 from AAV. In some embodiments, the endonuclease comprises a mutation that reduces activity of the endonuclease compared to the endonuclease without the mutation. In some embodiments, the endonuclease is an ORF2p endonuclease and the mutation is S228P. In some embodiments, the mRNA comprises a sequence encoding a domain that increases fidelity and/or processivity of the reverse transcriptase. In some embodiments, the reverse transcriptase is a reverse transcriptase from a retroelement other than ORF2 or reverse transcriptase that has higher fidelity and/or processivity compared to a reverse transcriptase of ORF2p. In some embodiments, the reverse transcriptase is a group II intron reverse transcriptase. In some embodiments, the group II intron reverse transcriptase is a group IIA intron reverse transcriptase, a group IIB intron reverse transcriptase, or a group IIC intron reverse transcriptase. In some embodiments, the group II intron reverse transcriptase is TGIRT-II or TGIRT-III.

In some embodiments, the mRNA comprises a sequence comprising an Alu element and/or a ribosome binding aptamer. In some embodiments, the mRNA comprises a sequence encoding a polypeptide comprising a DNA binding domain. In some embodiments, the 3′ UTR sequence is derived from a viral 3′ UTR or a beta-globin 3′ UTR.

In one aspect, provided herein is a composition comprising a recombinant mRNA or vector encoding an mRNA, wherein the mRNA comprises a human LINE-1 transposon sequence comprising a human LINE-1 transposon 5′ UTR sequence, a sequence encoding ORF1p downstream of the human LINE-1 transposon 5′ UTR sequence, an inter-ORF linker sequence downstream of the sequence encoding ORF1p,a sequence encoding ORF2p downstream of the inter-ORF linker sequence, and a 3′ UTR sequence derived from a human LINE-1 transposon downstream of the sequence encoding ORF2p; wherein the 3′ UTR sequence comprises an insert sequence, wherein the insert sequence is a reverse complement of a sequence encoding an exogenous polypeptide or a reverse complement of a sequence encoding an exogenous regulatory element.

In some embodiments, the insert sequence integrates into the genome of a cell when introduced into the cell. In some embodiments, the insert sequence integrates into a gene associated a condition or disease, thereby disrupting the gene or downregulating expression of the gene. In some embodiments, the insert sequence integrates into a gene, thereby upregulating expression of the gene. In some embodiments, the recombinant mRNA or vector encoding the mRNA is isolated or purified.

In one aspect, provided herein is a composition comprising a nucleic acid comprising a nucleotide sequence encoding (a) a long interspersed nuclear element (LINE) polypeptide, wherein the LINE polypeptide includes human ORF1p and human ORF2p; and (b) an insert sequence, wherein the insert sequence is a reverse complement of a sequence encoding an exogenous polypeptide or a reverse complement of a sequence encoding an exogenous regulatory element, wherein the composition is substantially non-immunogenic. In some embodiments, integration of the insert sequence does not adversely affect cell health.

In some embodiments, the composition comprises human ORF1p and human ORF2p proteins. In some embodiments, the composition comprises a ribonucleoprotein (RNP) comprising human ORF1p and human ORF2p complexed to the nucleic acid. In some embodiments, the nucleic acid is mRNA.

In one aspect, provided herein is a composition comprising a cell comprising a composition described herein. In some embodiments, the cell is an immune cell. In some embodiments, the immune cell is a T cell or a B cell. In some embodiments, the immune cell is a myeloid cell. In some embodiments, the immune cell is selected from a group consisting of a monocyte, a macrophage, a dendritic cell, a dendritic precursor cell, and a macrophage precursor cell. In some embodiments, the insert sequence is a reverse complement of a sequence encoding an exogenous polypeptide and the exogenous polypeptide is a chimeric antigen receptor (CAR).

In one aspect, provided herein is a pharmaceutical composition comprising a composition described herein, and a pharmaceutically acceptable excipient. In some embodiments, the pharmaceutical composition is for use in gene therapy. In some embodiments, the pharmaceutical composition is for use in the manufacture of a medicament for treating a disease or condition. In some embodiments, the pharmaceutical composition is for use in treating a disease or condition. In one aspect, provided herein is a method of treating a disease in a subject, comprising administering a pharmaceutical composition described herein to a subject with a disease or condition. In some embodiments, the method increases an amount or activity of a protein or functional RNA in the subject. In some embodiments, the subject has a deficient amount or activity of a protein or functional RNA. In some embodiments, the deficient amount or activity of a protein or functional RNA is associated with or causes the disease or condition.

In some embodiments, the method further comprising administering an agent that inhibits human silencing hub (HUSH) complex, an agent that inhibits FAM208A, or an agent that inhibits TRIM28. In some embodiments, the agent that inhibits human silencing hub (HUSH) complex is an agent that inhibits Periphilin, TASOR and/or MPP8. In some embodiments, the agent that inhibits human silencing hub (HUSH) complex inhibits assembly of the HUSH complex. In some embodiments, the agent inhibits the Fanconi anemia complex. In some embodiments, the agent inhibits FANCD2-FANC1 heterodimer monoubiquitylation. In some embodiments, the agent inhibits FANCD2-FANC1 heterodimer formation.

In some embodiments the agent inhibits the Fanconi Anemia (FA) core complex. FA core complex is a component of the Fanconi anemia DNA damage repair pathway, e.g., in chemotherapy induced DNA inter-strand crosslinks. The FA core complex comprises two central dimers of the FANCB and FA-associated protein of 100 kDa (FAAP100) subunits, flanked by two copies of the RING finger subunit, FANCL. These two heterotrimers act as a scaffold to assemble the remaining five subunits, resulting in an extended asymmetric structure. Destabilization of the scaffold would disrupt the entire complex. resulting in a non-functional FA pathway. Examples of agents that can inhibit the FA core complex include Bortezomib and curcumin analogs EF24 and 4H-TTD.

Accordingly, it is an object of the present invention to provide novel transposon-based vectors useful in providing gene therapy to an animal. It is an object of the present invention to provide novel transposon-based vectors for use in the preparation of a medicament useful in providing gene therapy to an animal or human. It is another object of the present invention to provide novel transposon-based vectors that encode for the production of desired proteins or peptides in cells. Yet another object of the present invention to provide novel transposon-based vectors that encode for the production of desired nucleic acids in cells. It is a further object of the present invention to provide methods for cell and tissue specific incorporation of transposon-based DNA or RNA constructs comprising targeting a selected gene to a specific cell or tissue of an animal. It is yet another object of the present invention to provide methods for cell and tissue specific expression of transposon-based DNA or RNA constructs comprising designing a DNA or RNA construct with cell specific promoters that enhance stable incorporation of the selected gene by the transposase and expressing the selected gene in the cell. It is an object of the present invention to provide gene therapy for generations through germ line administration of a transposon-based vector.

Another object of the present invention is to provide gene therapy in animals through non germ line administration of a transposon-based vector. Another object of the present invention is to provide gene therapy in animals through administration of a transposon-based vector, wherein the animals produce desired proteins, peptides or nucleic acids. Yet another object of the present invention is to provide gene therapy in animals through administration of a transposon-based vector, wherein the animals produce desired proteins or peptides that are recognized by receptors on target cells. Still another object of the present invention is to provide gene therapy in animals through administration of a transposon-based vector, wherein the animals produce desired fusion proteins or fusion peptides, a portion of which are recognized by receptors on target cells, in order to deliver the other protein or peptide component of the fusion protein or fusion peptide to the cell to induce a biological response. Yet another object of the present invention is to provide a method for gene therapy of animals through administration of transposon-based vectors comprising tissue specific promoters and a gene of interest to facilitate tissue specific incorporation and expression of a gene of interest to produce a desired protein, peptide or nucleic acid. Another object of the present invention is to provide a method for gene therapy of animals through administration of transposon-based vectors comprising cell specific promoters and a gene of interest to facilitate cell specific incorporation and expression of a gene of interest to produce a desired protein, peptide or nucleic acid. Still another object of the present invention is to provide a method for gene therapy of animals through administration of transposon-based vectors comprising cell specific promoters and a gene of interest to facilitate cell specific incorporation and expression of a gene of interest to produce a desired protein, peptide or nucleic acid, wherein the desired protein, peptide or nucleic acid has a desired biological effect in the animal.

In one aspect, provided herein are methods and compositions for delivery inside a cell, for example a myeloid cell and stable incorporation of one or more nucleic acids, comprising nucleic acid sequences encoding one or more proteins, wherein the stable incorporation may be via non-viral mechanisms. In some embodiments, the delivery of a nucleic acid composition into a myeloid cell is via a non-viral mechanism. In some embodiments, the delivery of the nucleic acids may further bypass plasmid mediated delivery. A “plasmid,” as used herein, refers to a non-viral expression vector, e.g., a nucleic acid molecule that encodes for genes and/or regulatory elements necessary for the expression of genes. A “viral vector,” as used herein, refers to a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

In some embodiments, provided herein is a method of delivering a composition inside a cell, such as in a myeloid cell, the composition comprising one or more nucleic acid sequences encoding one or more proteins, wherein the one or more nucleic acid sequences is an RNA. In some embodiments, the RNA is mRNA.

In some embodiments, one or more mRNA comprising one or more nucleic acid sequences are delivered. In some embodiments, the one or more mRNA may comprise at least one modified nucleotide. The term “nucleotide,” as used herein, refers to a base-sugar-phosphate combination. A nucleotide may comprise a synthetic nucleotide. A nucleotide may comprise a synthetic nucleotide analog. Nucleotides may be monomeric units of a nucleic acid sequence (e.g. deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, or derivatives thereof. Such derivatives may include, for example, [aS]dATP, 7-deaza-dGTP and 7-deaza-dATP, and nucleotide derivatives that confer nuclease resistance on the nucleic acid molecule containing them. The term nucleotide as used herein may refer to dideoxyribonucleoside triphosphates (ddNTPs) and their derivatives. Illustrative examples of dideoxyribonucleoside triphosphates may include, but are not limited to, ddATP, ddCTP, ddGTP, ddITP, and ddTTP. A nucleotide may be unlabeled or detectably labeled by well-known techniques. Labeling may also be carried out with quantum dots. Detectable labels may include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,NcN′-tetramethyl-6-carboxyrhodamine (TAMRA), 6-carboxy-X-rhodamine (ROX), 4-(4′dimethylaminophenylazo)benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides may include [R6G]dUTP, [TAMRA]dUTP, [RI1O]dCTP, [R6G]dCTP, [TAMRA]dCTP, [JOE]ddATP, [R6G]ddATP, [FAM]ddCTP, [R110]ddCTP, [TANIRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-I2-dUTP, Tetramethyl-rodamine-6-dUTP, TR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-1 4-UTP, BODIPY-FL-4-UTP, BODIPY-TMR-i4-UTP, BODIPY-TMR-14-dUTP, BODIPY-TR-i4-UTP, BODIPY-TR-14-dUTP, Cascade Blue-7-UTP, Cascade Blue-7-dUTP, fluorescein-12-UTP, fluorescein-I2-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides may also be labeled or marked by chemical modification.

A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-ii-cICTP, biotin-14-dCTP), and biotin-dUTP (e.g. biotin-11-dUTP, biotin-1.6-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure, and may perform any function, known or unknown. A polynucleotide may comprise one or more analogs (e.g. altered backbone, sugar, or nucleobase). If present, modifications to the nucleotide structure may be imparted before or after assembly of the polymer. Some non-limiting examples of modified nucleotides or analogs include: pseudouridine, 5-bromouracil, 5-methylcytosine, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, eDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides may be interrupted by non-nucleotide components.

In some embodiments, the nucleic acid composition may comprise one or more mRNA, comprising at least one mRNA encoding a transmembrane receptor implicated in an immune response function (e.g. a phagocytic receptor or synthetic chimeric antigen receptor) into human macrophage or dendritic cell or a suitable myeloid cell or a myeloid precursor cell. In some embodiments, the nucleic acid composition comprises one or more mRNA, and one or more lipids for delivery of the nucleic acid into a cell of hematopoietic origin, such as a myeloid cell or a myeloid cell precursor cell. In some embodiments, the one or more lipids may form a liposomal complex.

As used herein, the composition described herein may be used for delivery inside a cell. A cell may originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell may not be originating from a natural organism (e.g. a cell may be a synthetically made, sometimes termed an artificial cell). In some embodiments, the cell referred to herein is a mammalian cell. In some embodiments, the cell is a human cell. The methods and compositions described herein relates to incorporating a genetic material in a cell, more specifically a human cell, wherein the human cell can be any human cell. As used herein, a human cell may be of any origin, for example, a somatic cell, a neuron, a fibroblast, a muscle cell, an epithelial cell, a cardiac cell, or a hematopoietic cell. The methods and compositions described herein can also be applicable to and useful for incorporating exogenous nucleic acid in hard-to-transfect human cell. The methods are simple and universally applicable once a suitable exogenous nucleic acid construct has been designed and developed.

The methods and compositions described herein are applicable to incorporate an exogenous nucleic acid in a cell ex vivo. In some embodiments, the compositions may be applicable for systemic administration in an organism, where the nucleic acid material in the composition may be taken up by a cell in vivo, whereupon it is incorporated in cell in vivo.

In some embodiments, the methods and compositions described herein may be directed to incorporating an exogenous nucleic acid in a human hematopoietic cell, for example, a human cell of hematopoietic origin, such as a human myeloid cell or a myeloid cell precursor. However, the methods and compositions described herein can be used or made suitable for use in any biological cell with minimum modifications. Therefore, a cell as may refer to any cell that is a basic structural, functional and/or biological unit of a living organism.

In one aspect, provided herein are methods and compositions for utilizing transposable elements for stable incorporation of one or more nucleic acids into the genome of a cell, where the cell is a member of a hematopoietic cells, for example a myeloid cell. In some embodiments, the one or more nucleic acids comprise at least one nucleic acid sequence encoding a transmembrane receptor protein having a role in immune response. In some embodiments, the methods and compositions are directed to using a retrotransposable element for incorporating one or more nucleic acid sequences into a myeloid cell. The nucleic acid composition may comprise one or more nucleic sequences, such as a gene, where the gene is a transgene. The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and may include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene may refer to an “endogenous gene” or a native gene in its natural location in the genome of an organism. A gene may refer to an “exogenous gene” or a non-native gene. A non-native gene may refer to a gene not normally found in the host organism, but which is introduced into the host organism by gene transfer. A non-native gene may also refer to a gene not in its natural location in the genome of an organism. A non-native gene may also refer to a naturally occurring nucleic acid or polypeptide sequence that comprises mutations, insertions and/or deletions (e.g., non-native sequence).

The term “transgene” refers to any nucleic acid molecule that is introduced into a cell, that may be intermittently termed herein as a recipient cell. The resultant cell after receiving a transgene may be referred to a transgenic cell. A transgene may include a gene that is partly or entirely heterologous (i.e., foreign) to the transgenic organism or cell, or may represent a gene homologous to an endogenous gene of the organism or cell. In some cases, transgenes include any polynucleotide, such as a gene that encodes a polypeptide or protein, a polynucleotide that is transcribed into an inhibitory polynucleotide, or a polynucleotide that is not transcribed (e.g., lacks an expression control element, such as a promoter that drives transcription). Transcripts and encoded polypeptides may be collectively referred to as “gene product.” If the polynucleotide is derived from genomic DNA, expression may include splicing of the mRNA in a eukaryotic cell. “Up-regulated,” with reference to expression, refers to an increased expression level of a polynucleotide (e.g., RNA such as mRNA) and/or polypeptide sequence relative to its expression level in a wild-type state while “down-regulated” refers to a decreased expression level of a polynucleotide (e.g., RNA such as mRNA) and/or polypeptide sequence relative to its expression in a wild-type state. Expression of a transfected gene may occur transiently or stably in a cell. During “transient expression” the transfected gene is not transferred to the daughter cell during cell division. Since its expression is restricted to the transfected cell, expression of the gene is lost over time. In contrast, stable expression of a transfected gene may occur when the gene is co-transfected with another gene that confers a selection advantage to the transfected cell. Such a selection advantage may be a resistance towards a certain toxin that is presented to the cell. Where a transfected gene is required to be expressed, the application envisages the use of codon-optimized sequences. An example of a codon optimized sequence may be a sequence optimized for expression in a eukaryote, e.g., humans (i.e. being optimized for expression in humans), or for another eukaryote, animal or mammal.

Codon optimization for a host species other than human, or for codon optimization for specific organs is known. In some embodiments, the coding sequence encoding a protein may be codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. Codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell may generally reflect the codons used most frequently in peptide synthesis. Accordingly, genes may be tailored for optimal gene expression in a given organism based on codon optimization. Codon usage tables are readily available, for example, at the “Codon Usage Database” available at www.kazusa.orjp/codon/and these tables may be adapted in a number of ways. Computer algorithms for codon optimizing a particular sequence for expression in a particular host cell are also available, such as Gene Forge (Aptagen; Jacobus, PA), are also available.

A “multicistronic transcript” as used herein refers to an mRNA molecule that contains more than one protein coding region, or cistron. A mRNA comprising two coding regions is denoted a “bicistronic transcript.” The “5′-proximal” coding region or cistron is the coding region whose translation initiation codon (usually AUG) is closest to the 5′ end of a multicistronic mRNA molecule. A “5′-distal” coding region or cistron is one whose translation initiation codon (usually AUG) is not the closest initiation codon to the 5′ end of the mRNA.

The terms “transfection” or “transfected” refer to introduction of a nucleic acid into a cell by non-viral or viral-based methods. The nucleic acid molecules may be gene sequences encoding complete proteins or functional portions thereof. See, e.g., Sambrook et al., 1989, Molecular Cloning: A Laboratory Manual, 18.1-18.88.

The term “promoter,” as used herein, refers to a polynucleotide sequence capable of driving transcription of a coding sequence in a cell. Thus, promoters used in the polynucleotide constructs of the disclosure include cis-acting transcriptional control elements and regulatory sequences that are involved in regulating or modulating the timing and/or rate of transcription of a gene. For example, a promoter may be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions, developmental conditions, or drug or chemical conditions. Exemplary inducible promoter may be a doxycycline or a tetracycline inducible promoter. Tetracycline regulated promoters may be both tetracycline inducible or tetracycline repressible, called the tet-on and tet-off systems. The tet regulated systems rely on two components, i.e., a tetracycline-controlled regulator (also referred to as transactivator) (tTA or rtTA) and a tTA/rtTA-dependent promoter that controls expression of a downstream cDNA, in a tetracycline-dependent manner. tTA is a fusion protein containing the repressor of the Tn10 tetracycline-resistance operon of Escherichia coli and a carboxyl-terminal portion of protein 16 of herpes simplex virus (VP16).

The tTA-dependent promoter consists of a minimal RNA polymerase II promoter fused to tet operator (tetO) sequences (an array of seven cognate operator sequences). This fusion converts the tet repressor into a strong transcriptional activator in eukaryotic cells. In the absence of tetracycline or its derivatives (such as doxycycline), tTA binds to the tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. In contrast, in the tet-ON system, a mutant form of tTA, termed rtTA, has been isolated using random mutagenesis. In contrast to tTA, rtTA is not functional in the absence of doxycycline but requires the presence of the ligand for transactivation. The term “exon” refers to a nucleic acid sequence found in genomic DNA that is bioinformatically predicted and/or experimentally confirmed to contribute contiguous sequence to a mature mRNA transcript. The term “intron” refers to a sequence present in genomic DNA that is bioinformatically predicted and/or experimentally confirmed to not encode part of or all of an expressed protein, and which, in endogenous conditions, is transcribed into RNA (e.g. pre-mRNA) molecules, but which is spliced out of the endogenous RNA (e.g. the pre-mRNA) before the RNA is translated into a protein.

The term “splice acceptor site” refers to a sequence present in genomic DNA that is bioinformatically predicted and/or experimentally confirmed to be the acceptor site during splicing of pre-mRNA, which may include identified and unidentified natural and artificially derived or derivable splice acceptor sites.

An “internal ribosome entry site” or “IRES” refers to a nucleotide sequence that allows for 5′-end/cap-independent initiation of translation and thereby raises the possibility to express 2 proteins from a single messenger RNA (mRNA) molecule. IRESs are commonly located in the 5′ UTR of positive-stranded RNA viruses with uncapped genomes. Another means to express 2 proteins from a single mRNA molecule is by insertion of a 2A peptide(-like) sequence in between their coding sequence. 2A peptide(-like) sequences mediate self-processing of primary translation products by a process variously referred to as “ribosome skipping”, “stop-go” translation and “stop carry-on” translation. 2A peptide(-like) sequences are present in various groups of positive- and double-stranded RNA viruses including Picornaviridae, Flaviviridae, Tetraviridae, Dicistroviridae, Reoviridae and Totiviridae.

The term “2A peptide” refers to a class of 18-22 amino-acid (AA)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” is believed to be ribosome skipping the formation of a glycyl-prolyl peptide bond at the C-terminus of the 2A sequence. 2A peptide(-like) sequences mediate self-processing of primary translation products by a process variously referred to as “ribosome skipping”, “stop-go” translation and “stop carry-on” translation. 2A peptide(-like) sequences are present in various groups of positive- and double-stranded RNA viruses including Picornaviridae, Flaviviridae, Tetraviridae, Dicistroviridae, Reoviridae and Totiviridae.

As used herein, the term “operably linked” refers to a functional relationship between two or more segments, such as nucleic acid segments or polypeptide segments. Typically, it refers to the functional relationship of a transcriptional regulatory sequence to a transcribed sequence.

The term “termination sequence” refers to a nucleic acid sequence which is recognized by the polymerase of a host cell and results in the termination of transcription. The termination sequence is a sequence of DNA that, at the 3′ end of a natural or synthetic gene, provides for termination of mRNA transcription or both mRNA transcription and ribosomal translation of an upstream open reading frame. Prokaryotic termination sequences commonly comprise a GC-rich region that has a two-fold symmetry followed by an AT-rich sequence. A commonly used termination sequence is the T7 termination sequence. A variety of termination sequences are known in the art and may be employed in the nucleic acid constructs of the present invention, including the TINT3, TL13, TL2, TR1, TR2, and T6S termination signals derived from the bacteriophage lambda, and termination signals derived from bacterial genes, such as the trp gene of E. coli.

The terms “polyadenylation sequence” (also referred to as a “poly A site” or “poly A sequence”) refers to a DNA sequence which directs both the termination and polyadenylation of the nascent RNA transcript. Efficient polyadenylation of the recombinant transcript is desirable, as transcripts lacking a poly A tail are typically unstable and rapidly degraded. The poly A signal utilized in an expression vector may be “heterologous” or “endogenous”. An endogenous poly A signal is one that is found naturally at the 3′ end of the coding region of a given gene in the genome. A heterologous poly A signal is one which is isolated from one gene and placed 3′ of another gene, e.g., coding sequence for a protein. A commonly used heterologous poly A signal is the SV40 poly A signal. The SV40 poly A signal is contained on a 237 bp BamHI/BcII restriction fragment and directs both termination and polyadenylation; numerous vectors contain the SV40 poly A signal. Another commonly used heterologous poly A signal is derived from the bovine growth hormone (BGH) gene; the BGH poly A signal is also available on a number of commercially available vectors. The poly A signal from the Herpes simplex virus thymidine kinase (HSV tk) gene is also used as a poly A signal on a number of commercial expression vectors. The polyadenylation signal facilitates the transportation of the RNA from within the cell nucleus into the cytosol as well as increases cellular half-life of such an RNA. The polyadenylation signal is present at the 3′-end of an mRNA.

The terms “complement,” “complements,” “complementary,” and “complementarity,” as used herein, refer to a sequence that is complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g. thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, may be measured by any suitable alignment algorithm, including but not limited to the Needleman-Wunsch algorithm (see e.g. the EMBOSS Needle aligner available at www.ebi.ac.uk/Tools/psa/embossneedle/nucleotide.html), the BLAST algorithm (see e.g. the BLAST alignment tool available at blast.ncbi.nlm.nih.gov/Blast.cgi, optionally with default settings), or the Smith-Waterman algorithm (see e.g. the EMBOSS Water aligner available at www.ebi.ac.ukaools/psa/emboss_water/nucleotide.html, optionally with default settings). Optimal alignment can be assessed using any suitable parameters of a chosen algorithm, including default parameters.

Complementarity may be perfect or substantial/sufficient. Perfect complementarity between two nucleic acids may mean that the two nucleic acids may form a duplex in which every base in the duplex is bonded to a complementary base by Watson-Crick pairing. Substantial or sufficient complementary may mean that, a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions may be predicted by using the sequences and standard mathematical calculations to predict the melting temperature (Tₘ) of hybridized strands, or by empirical determination of Tₘ by using routine methods. [00282]“Transposons” as used herein are segments within the chromosome that can translocate within the genome, also known as “jumping gene”. There are two different classes of transposons: class 1, or retrotransposons, that mobilize via an RNA intermediate and a “copy-and-paste” mechanism, and class II, or DNA transposons, that mobilize via excision integration, or a “cut-and-paste” mechanism (Ivics Nat Methods 2009). Bacterial, lower eukaryotic (e.g. yeast) and invertebrate transposons appear to be largely species specific, and cannot be used for efficient transposition of DNA in vertebrate cells. “Sleeping Beauty” (Ivics Cell 1997), was the first active transposon that was artificially reconstructed by sequence shuffling of inactive TEs from fish. This made it possible to successfully achieve DNA integration by transposition into vertebrate cells, including human cells. Sleeping Beauty is a class II DNA transposon belonging to the Tcl/mariner family of transposons (Ni Genomics Proteomics 2008). In the meantime, additional functional transposons have been identified or reconstructed from different species, including Drosophila, frog and even human genomes, that all have been shown to allow DNA transposition into vertebrate and also human host cell genomes. Each of these transposons have advantages and disadvantages that are related to transposition efficiency, stability of expression, genetic payload capacity etc. Exemplary class II transposases that have been created include Sleeping Beauty, PiggyBac, Frog Prince, Himarl, Passport, Minos, hAT, Toll, To12, AciDs, PIF, Harbinger, Harbinger3-DR, and Hsmarl.

“Heterologous” as used herein, includes molecules such as DNA and RNA which may not naturally be found in the cell into which it is inserted. For example, when mouse or bacterial DNA is inserted into the genome of a human cell, such DNA is referred to herein as heterologous DNA. In contrast, the term “homologous” as used herein, denotes molecules such as DNA and RNA that are found naturally in the cell into which it is inserted. For example, the insertion of mouse DNA into the genome of a mouse cell constitutes insertion of homologous DNA into that cell. In the latter case, it is not necessary that the homologous DNA be inserted into a site in the cell genome in which it is naturally found; rather, homologous DNA may be inserted at sites other than where it is naturally found, thereby creating a genetic alteration (a mutation) in the inserted site.

A “transposase” is an enzyme that is capable of forming a functional complex with a transposon end-containing composition (e.g., transposons, transposon ends), and catalyze insertion or transposition of the transposon end-containing composition into double stranded DNA which is incubated with an in vitro transposon reaction. The term “transposon end” means a double-stranded DNA that contains the nucleotide sequences (the “transposon end sequences”) necessary to form the complex with the transposase or integrase enzyme that is functional in an in vitro transposition reaction.

A transposon end forms a complex or a synaptic complex or a transposon complex or a transposon composition with a transposase or integrase that recognizes and binds to the transposon end, and which complex is capable of inserting or transposing the transposon end into target DNA with which it is incubated in an in vitro transposition reaction. A transposon end exhibits two complementary sequences consisting of a transferred transposon end sequence or transferred strand and a non-transferred transposon end sequence, or non-transferred strand For example, one transposon end that forms a complex with a hyperactive Tn5 transposase that is active in an in vitro transposition reaction comprises a transferred strand that exhibits a transferred transposon end sequence as follows: 5′ AGATGTGTATAAGAGACAG 3′ (SEQ ID NO: 55), and a non-transferred strand that exhibits a “non-transferred transposon end sequence” as follows: (5′ CTGTCTCTTATACACATCT 3′ (SEQ ID NO: 56). The 3′-end of a transferred strand is joined or transferred to target DNA in an in vitro transposition reaction. The non-transferred strand, which exhibits a transposon end sequence that is complementary to the transferred transposon end sequence, is not joined or transferred to the target DNA in an in vitro transposition reaction.

In some embodiments, the transferred strand and non-transferred strand are covalently joined. For example, in some embodiments, the transferred and non-transferred strand sequences are provided on a single oligonucleotide, e.g., in a hairpin configuration. As such, although the free end of the non-transferred strand is not joined to the target DNA directly by the transposition reaction, the non-transferred strand becomes attached to the DNA fragment indirectly, because the non-transferred strand is linked to the transferred strand by the loop of the hairpin structure. As used herein an “cleavage domain” refers to a nucleic acid sequence that is susceptible to cleavage by an agent, e.g., an enzyme.

A “restriction site domain” means a tag domain that exhibits a sequence for the purpose of facilitating cleavage using a restriction endonuclease. For example, in some embodiments, the restriction site domain is used to generate di-tagged linear ssDNA fragments. In some embodiments, the restriction site domain is used to generate a compatible double-stranded 5′-end in the tag domain so that this end can be ligated to another DNA molecule using a template-dependent DNA ligase. In some embodiments, the restriction site domain in the tag exhibits the sequence of a restriction site that is present only rarely, if at all, in the target DNA (e.g., a restriction site for a rare-cutting restriction endonuclease such as NotI or AscI).

As used herein, the term “recombinant nucleic acid molecule” refers to a recombinant DNA molecule or a recombinant RNA molecule. A recombinant nucleic acid molecule is any nucleic acid molecule containing joined nucleic acid molecules from different original sources and not naturally attached together. Recombinant RNA molecules include RNA molecules transcribed from recombinant DNA molecules. A recombinant nucleic acid may be synthesized in the laboratory. A recombinant nucleic acid can be prepared by using recombinant DNA technology by using enzymatic modification of DNA, such as enzymatic restriction digestion, ligation, and DNA cloning. A recombinant DNA may be transcribed in vitro, to generate a messenger RNA (mRNA), the recombinant mRNA may be isolated, purified and used to transfect a cell. A recombinant nucleic acid may encode a protein or a polypeptide. A recombinant nucleic acid, under suitable conditions, can be incorporated into a living cell, and can be expressed inside the living cell. As used herein, “expression” of a nucleic acid usually refers to transcription and/or translation of the nucleic acid. The product of a nucleic acid expression is usually a protein but can also be an mRNA. Detection of an mRNA encoded by a recombinant nucleic acid in a cell that has incorporated the recombinant nucleic acid, is considered positive proof that the nucleic acid is “expressed” in the cell. The process of inserting or incorporating a nucleic acid into a cell can be via transformation, transfection or transduction. Transformation is the process of uptake of foreign nucleic acid by a bacterial cell. This process is adapted for propagation of plasmid DNA, protein production, and other applications. Transformation introduces recombinant plasmid DNA into competent bacterial cells that take up extracellular DNA from the environment. Some bacterial species are naturally competent under certain environmental conditions, but competence is artificially induced in a laboratory setting. Transfection is the forced introduction of small molecules such as DNA, RNA, or antibodies into eukaryotic cells. Just to make life confusing, ‘transfection’ also refers to the introduction of bacteriophage into bacterial cells. ‘Transduction’ is mostly used to describe the introduction of recombinant viral vector particles into target cells, while ‘infection’ refers to natural infections of humans or animals with wild-type viruses.

A “stem-loop” sequence refers to a nucleic acid sequence (e.g., RNA sequence) with sufficient self-complementarity to hybridize and form a stem and the regions of non-complementarity that bulges into a loop. The stem may comprise mismatches or bulges.

The term “vector” refers to a nucleic acid molecule capable of transporting or mediating expression of a heterologous nucleic acid. A “vector sequence” as used herein, refers to a sequence of nucleic acid comprising at least one origin of replication and at least one selectable marker gene. Vectors capable of directing the expression of genes and/or nucleic acid sequence to which they are operatively linked are referred to herein as “expression vectors”.

A plasmid is a species of the genus encompassed by the term “vector.” In general, expression vectors of utility are often in the form of “plasmids” which refer to circular double stranded DNA molecules which, in their vector form are not bound to the chromosome, and typically comprise entities for stable or transient expression of the encoded DNA. Other expression vectors that can be used in the methods as disclosed herein include, but are not limited to plasmids, episomes, bacterial artificial chromosomes, yeast artificial chromosomes, bacteriophages or viral vectors, and such vectors can integrate into the host's genome or replicate autonomously in the cell. A vector can be a DNA or RNA vector. Other forms of expression vectors known by those skilled in the art which serve the equivalent functions can also be used, for example, self-replicating extrachromosomal vectors or vectors capable of integrating into a host genome. Exemplary vectors are those capable of autonomous replication and/or expression of nucleic acids to which they are linked. A safe harbor locus is a region within the genome where additional exogenous or heterologous nucleic acid sequence can be inserted, and the host genome is able to accommodate the inserted genetic material. Exemplary safe harbor sites include but are not limited to: AAVS1 site, GGTA1 site, CMAH site, B4GALNT2 site, B2M site, ROSA26 site, COLA1 site, and TIGRE site. For example, the heterologous nucleic acid described in this disclosure may be integrated at one or more sites in the genome of the cell, wherein the one or more locations is selected from the group consisting of: AAVS1 site, GGTA1 site, CMAH site, B4GALNT2 site, B2M site, ROSA26 site, COLA1 site, and TIGRE site. In some embodiments, the nucleic acid cargo comprising the transgene may be delivered to a R2D locus.

In some embodiments, the nucleic acid cargo comprising the transgene may be delivered to the genome in an intergenic or intragenic region. In some embodiments the nucleic acid cargo comprising the transgene is integrated into the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous active gene. In some embodiments the nucleic acid cargo comprising the transgene is integrated into the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous promoter or enhancer. In some embodiments the nucleic acid cargo comprising the transgene is 50-50,000 base pairs, e.g., between 50-40,000 bp, between 500-30,000 bp between 500-20,000 bp, between 100-15,000 bp, between 500-10,000 bp, between 50-10,000 bp, between 50-5,000 bp.

In some embodiments the nucleic acid cargo comprising the transgene is less than 1,000, 1,300, 1500, 2,000, 3,000, 4,000, 5,000, or 7,500 nucleotides in length.

L1 and Non-L1 Retrotransposon Systems

Retrotransposons can contain transposable elements that are active participants in reorganizing their resident genomes. Broadly, retrotransposons can refer to DNA sequences that are transcribed into RNA and translated into protein and have the ability to reverse-transcribe themselves back into DNA.

Approximately 45% of the human genome is comprised of sequences that result from transposition events.

Retrotransposition occasionally generates target site deletions or adds non-retrotransposon DNA to the genome by processes termed 5′- and 3′-transduction. Recombination between non-homologous retrotransposons causes deletions, duplications or rearrangements of gene sequence. Ongoing retrotransposition can generate novel splice sites, polyadenylation signals and promoters, and so builds new transcription modules.

Generally, retrotransposons may be grouped into two classes, the retrovirus-like LTR retrotransposons, and the non-LTR elements such as human L1 elements, Neurospora TAD elements (Kinsey, 1990, Genetics 126:317-326), I factors from Drosophila (Bucheton et al., 1984, Cell 38:153-163), and R2Bm from Bombyx mori (Luan et al., 1993, Cell 72: 595-605). These two types of retrotransposons are structurally different and also retrotranspose using radically different mechanisms. Exemplary, non-limiting examples of LINE-encoded polypeptides are found in GenBank Accession Nos. AAC51261, AAC51262, AAC51263, AAC51264, AAC51265, AAC51266, AAC51267, AAC51268, AAC51269, AAC51270, AAC51271, AAC51272, AAC51273, AAC51274, AAC51275, AAC51276, AAC51277, AAC51278 and AAC51279.

The decision to focus on LINE-1 to develop into a system as described in the disclosure for a number of reasons at least some of which are exemplified below: (a) LINE-1 (or L1-) elements are autonomous as they encode all of the machinery alone to complete this reverse transcription and integration process; (b) L1 elements are abundant in the human genome, such that these elements may be considered as a naturalized element of the genome; (c) L1 retrotransposon retrotransposes its own mRNA with high degree of specificity, compared to other mRNAs floating around in the cells.

The L1 expresses a 6-kb bicistronic RNA that encodes the 40 kDa Open Reading Frame-1 RNA-binding protein (ORF1p) of essential but uncertain function, and a 150 kDa ORF2 protein with endonuclease and reverse transcriptase (RT) activities. L1 retrotransposition is a complex process involving transcription of the L1, transport of its RNA to the cytoplasm, translation of the bicistronic RNA, formation of a ribonucleoprotein (RNP) particle, its re-import to the nucleus and target-primed reverse transcription at the integration site. A few transcription factors that interact with L1s have been identified.

Transcribed L1 RNA forms an RNP in cis with the proteins that are translated from the transcript. L1 integrates into genomic DNA by target-site primer reverse transcription (TPRT) by ORF2p cleavage at the 5′-TTTT-3′ where a poly A sequence of L1 RNA anneals and primes reverse transcriptase (RT) activity to make L1 cDNA.

Other mobile elements of the genome can “hijack” the L1 ORF for retrotransposition. For example, Alu elements are such mobile DNA elements that belong to the class of short interspersed elements (SINEs) that are non-autonomous retrotransposons and acquire trans-factors to integrate. Alu elements and SINE-1 elements can associate with the L1 ribonucleoproteins in trans to be also retrotransposed by ORF1p and ORF2p. Somewhat similar to the L1 RNA, the Alu element ends with a long A-run, often referred to as the A-tail, and it also has a smaller A-rich region (indicated by AA) separating the two halves of a diverged dimer structure. Alu elements are likely to have the internal components of an RNA polymerase III promoter (such as, commonly designated as an A box and a B box promoters), but they do not encode a terminator for RNA polymerase III. They may utilize a stretch of T nucleotides at various distances downstream of the Alu element to terminate a transcription. A typical Alu transcript encompasses the entire Alu, including the A-tail, and has a 3′ region that is unique for each locus. The Alu RNA folds into separate structures for each monomer unit. The RNA has been shown to bind the 7SL RNA SRP9 and 14 heterodimer, as well as poly A-binding protein (PABP). The poly A tail of Alu primes with T rich (TTTT) region of the genome and attracts ORF2p to bind to the primed region and cleaves at the T rich region via its endonuclease activity. The T-rich region primes reverse transcription by ORF2p on the 3′ A-tail region of the Alu element. This creates a cDNA copy of the body of the Alu element.

A nick occurs by an unknown mechanism on the second strand and second-strand synthesis is primed. The new Alu element is then flanked by short direct repeats that are duplicates of the DNA sequence between the first and second nicks. Alu elements are extremely prevalent within RNA molecules, owing to their preference for gene-rich regions. A full-length Alu (300 bp) is derived from the signal recognition particle RNA 7SL and consists of two similar monomers with an A-rich linker in-between, A- and B-boxes present in the 5′ monomer, and a poly-A tail lacking the preceding polyadenylation signal resulting in an elongated tail (up to 100 bp in length). Alus can be transcribed by RNA polymerase III using the internal promoters within the A- and B-boxes; however, Alus contain no ORFs and therefore do not encode for protein products.

Other non-L1 transposons include SVAs and HERV-Ks. A full-length SVA (SINE-VNTR-Alu) element (˜2-3 kb) is a composite unit that contains a CCCTCT repeat, two Alu-like sequences, a VNTR, a SINE-R region with env (envelope) gene, the 3′ LTR of HERV-K10, and a polyadenylation signal followed by a poly-A tail. It is most likely that SVAs are transcribed by RNA polymerase II, although it is unknown whether SVA elements carry an internal promoter.

A full-length HERV-K element (˜9-10 kb) is comprised of ancient remnants of endogenous retroviral sequences and includes two flanking LTR regions surrounding three retroviral ORFs: (1) gag encoding the structural proteins of a retroviral capsid; (2) pol-pro encoding the enzymes: protease, RT, and integrase; and (3) env encoding proteins allowing for horizontal transfer. The LTR of HERV-K contains an internal, bidirectional promoter that appears to be under the transcriptional control of RNA polymerase II.

L1 retrotransposition and RNA binding can take place at or near poly-A tail. The 3′-UTR plays a role in the recognition of stringent-type LINE RNA of ORF1 protein (ORF1p). Stringent-type LINEs can contain a stem-loop structure located at the end of the 3′UTR. Branched molecules consisting of junctions between transposon 3′-end cDNA and the target DNA, as well as specific positioning of L1 RNA within ORF2 protein (ORF2p), were detected during initial stages of L1 retrotransposition in vitro. Secondary or tertiary RNA structure shared by L1 and Alu are likely to be responsible for recognition by and binding of ORF2, possibly along with a poly-A tail. In some embodiments, the stem-loop structure located downstream of the poly-A sequence correlates with cleavage intensity.

Mechanisms for restricting or resolving L1 integration have also evolved for the sake of maintaining genetic integrity and stability of the genome. Non-homologous end-joining repair proteins, such as XRCC1, Ku70 and DNA-PK, have been implicated in resolution of the L1 integrate at the time of insertion. In addition, the cell has evolved a number of proteins that stand against unrestricted retrotransposition, including the APOBEC3 family of cytosine deaminases, adenosine deaminase ADAR1, chromatin-remodeling factors and members of the piRNA pathway for post-transcription gene silencing that functions in the male germ line.

### L. Compositions Comprising Nucleic Acid Constructs and Methods Involved for Stable Expression of Encoded Protein

Provided herein is a recombinant nucleic acid encoding one or more proteins for expression in a cell, such as a myeloid cell. In one embodiment, the recombinant nucleic acid is designed for stable expression of the one or more proteins or polypeptides encoded by the recombinant nucleic acid. In some embodiments, the stable expression is achieved by incorporation of recombinant nucleic acid within the genome of the cell.

It can be easily understood by one of skill in the art that the compositions and methods described herein can be utilized to design products in which the recombinant nucleic acid may comprise one or more sequences that do not translate as a protein or a polypeptide component, but may encode an oligonucleotide that can be a regulatory nucleic acid, such as an inhibitor oligonucleotide product, such as an activator oligonucleotide.

In one aspect, provided herein is a composition comprising a synthetic nucleic acid, comprising a nucleic acid sequence encoding a gene of interest and one or more retrotransposable elements to stably incorporate a non-endogenous nucleic acid into a cell. In some embodiments, the cell is a hematopoietic cell. In some embodiments, the cell is a myeloid cell. In some embodiments, the cell is a precursor cell. In some embodiments, the cell is undifferentiated. In some embodiments, the cell has further differentiation potential. In some embodiments, the cell is not a stem cell.

### A. LINE/Alu Retrotransposon Construct

In some embodiments, the present disclosure may utilize a retrotransposable system to stably incorporate into the genome and express a non-endogenous nucleic acid, where the non-endogenous nucleic acid comprises retrotransposable elements within the nucleic acid sequence. In some embodiments, the present disclosure may utilize a cell's endogenous retrotransposable system (e.g., proteins and enzymes), to stably express a non-endogenous nucleic acid in the cell. In some embodiments, the present disclosure may utilize a cell's endogenous retrotransposable system (e.g., proteins and enzymes, such as a LINE1 retrotransposition system), but may further express one or more components of the retrotransposable system to stably express a non-endogenous nucleic acid in the cell.

In some embodiments, a synthetic nucleic acid is provided herein, the synthetic nucleic acid encoding a transgene, and encoding one or more components for retrotransposition. The synthetic nucleic acid described herein is interchangeably termed as a nucleic acid construct, transgene or the exogenous nucleic acid.

In one aspect, provided herein is a method of integrating a nucleic acid sequence into a genome of a cell, the method comprising introducing a recombinant mRNA or a vector encoding an mRNA into the cell, wherein the mRNA comprises: an insert sequence, wherein the insert sequence comprises an exogenous sequence, or a sequence that is a reverse complement of the exogenous sequence; a 5′ UTR sequence and a 3′ UTR sequence downstream of the 5′ UTR sequence; wherein the 5′ UTR sequence or the 3′ UTR sequence comprises a binding site for a human ORF protein, and wherein the insert sequence is integrated into the genome of the cell.

In some embodiments, the 5′ UTR sequence or the 3′ UTR sequence comprises a binding site for human ORF2p.

In one aspect, provided herein is a method for integrating a nucleic acid sequence into the genome of an immune cell, the method comprising introducing a recombinant mRNA or a vector encoding an mRNA, wherein the mRNA comprises an insert sequence, wherein the insert sequence comprises (i) an exogenous sequence or (ii) a sequence that is a reverse complement of the exogenous sequence; 5′ UTR sequence and a 3′ UTR sequence downstream of the 5′ UTR sequence, wherein the 5′ UTR sequence or the 3′ UTR sequence comprises an endonuclease binding site and/or a reverse transcriptase binding site, and wherein the transgene sequence is integrated into the genome of the immune cell.

In one aspect, provided herein is a method for integrating a nucleic acid sequence into the genome of a cell, the method comprising introducing a recombinant mRNA or a vector encoding an mRNA, wherein the mRNA comprises an insert sequence, wherein the insert sequence comprises (i) an exogenous sequence or (ii) a sequence that is a reverse complement of the exogenous sequence; a 5′ UTR sequence, a sequence of a human retrotransposon downstream of the 5′ UTR sequence, and a 3′ UTR sequence downstream of the sequence of a human retrotransposon; wherein the 5′ UTR sequence or the 3′ UTR sequence comprises an endonuclease binding site and/or a reverse transcriptase binding site, and wherein the sequence of a human retrotransposon encodes for two proteins that are translated from a single RNA containing two ORFs, and wherein the insert sequence is integrated into the genome of the cell.

In some embodiments, the 5′ UTR sequence or the 3′ UTR sequence comprises an ORF2p binding site. In some embodiments, the ORF2p binding site is a poly A sequence in the 3′ UTR sequence.

In some embodiments, the mRNA comprises a sequence of a human retrotransposon. In some embodiments, the sequence of a human retrotransposon is downstream of the 5′ UTR sequence. In some embodiments, the sequence of a human retrotransposon is upstream of the 3′ UTR sequence.

In some embodiments, the sequence of a human retrotransposon encodes for two proteins that are translated from a single RNA containing two ORFs. In some embodiments, the two ORFs are non-overlapping ORFs. In some embodiments, the two ORFs are ORF1 and ORF2. In some embodiments, the ORF1 encodes ORF1p and ORF2 encodes ORF2p.

In some embodiments, the sequence of a human retrotransposon comprises a sequence of a non-LTR retrotransposon. In some embodiments, the sequence of a human retrotransposon encodes comprises a LINE-1 retrotransposon. In some embodiments, the LINE-1 retrotransposon is a human LINE-1 retrotransposon. In some embodiments, the sequence of a human retrotransposon comprises a sequence encoding an endonuclease and/or a reverse transcriptase. In some embodiments, the endonuclease and/or a reverse transcriptase is ORF2p. In some embodiments, the reverse transcriptase is a group II intron reverse transcriptase domain. In some embodiments, the endonuclease and/or a reverse transcriptase is a minke whale endonuclease and/or a reverse transcriptase. In some embodiments, the sequence of a human retrotransposon comprises a sequence encoding ORF2p. In some embodiments, the insert sequence is integrated into the genome at a poly T site using specificity of an endonuclease domain of the ORF2p. In some embodiments, the poly T site comprises the sequence TTTTTA.

In some embodiments, (i) the sequence of a human retrotransposon comprises a sequence encoding ORF1p, (ii) the mRNA does not comprise a sequence encoding ORF1p, or (iii) the mRNA comprises a replacement of the sequence encoding ORF1p with a 5′ UTR sequence from the complement gene. In some embodiments, the mRNA comprises a first mRNA molecule encoding ORF1p, and a second mRNA molecule encoding an endonuclease and/or a reverse transcriptase. In some embodiments, the mRNA is an mRNA molecule comprising a first sequence encoding ORF1p, and a second sequence encoding an endonuclease and/or a reverse transcriptase. In some embodiments, the first sequence encoding ORF1p and the second sequence encoding an endonuclease and/or a reverse transcriptase are separated by a linker sequence.

In some embodiments, the linker sequence comprises an internal ribosome entry sequence (IRES).

In some embodiments, the IRES is an IRES from CVB3 or EV71. In some embodiments, the linker sequence encodes a self-cleaving peptide sequence. In some embodiments, the linker sequence encodes a T2A, a E2A or a P2A sequence

In some embodiments, the sequence of a human retrotransposon comprises a sequence that encodes ORF1p fused to an additional protein sequence and/or a sequence that encodes ORF2p fused to an additional protein sequence. In some embodiments, the ORF1p and/or the ORF2p is fused to a nuclear retention sequence. In some embodiments, the nuclear retention sequence is an Alu sequence. In some embodiments, the ORF1p and/or the ORF2p is fused to an MS2 coat protein. In some embodiments, the 5′ UTR sequence or the 3′ UTR sequence comprises at least one, two, three or more MS2 hairpin sequences.

In some embodiments, the 5′ UTR sequence or the 3′ UTR sequence comprises a sequence that promotes or enhances interaction of a poly A tail of the mRNA with the endonuclease and/or a reverse transcriptase.

In some embodiments, the 5′ UTR sequence or the 3′ UTR sequence comprises a sequence that promotes or enhances interaction of a poly-A-binding protein (PABP) with the endonuclease and/or a reverse transcriptase. In some embodiments, the 5′ UTR sequence or the 3′ UTR sequence comprises a sequence that increases specificity of the endonuclease and/or a reverse transcriptase to the mRNA relative to another mRNA expressed by the cell. In some embodiments, the 5′ UTR sequence or the 3′ UTR sequence comprises an Alu element sequence.

In some embodiments, the first sequence encoding ORF1p and the second sequence encoding an endonuclease and/or a reverse transcriptase have the same promoter. In some embodiments, the insert sequence has a promoter that is different from the promoter of the first sequence encoding ORF1p. In some embodiments, the insert sequence has a promoter that is different from the promoter of the second sequence encoding an endonuclease and/or a reverse transcriptase. In some embodiments, the first sequence encoding ORF1p and/or the second sequence encoding an endonuclease and/or a reverse transcriptase have a promoter or transcription initiation site selected from the group consisting of an inducible promoter, a CMV promoter or transcription initiation site, a T7 promoter or transcription initiation site, an EF1a promoter or transcription initiation site and combinations thereof. In some embodiments, the insert sequence has a promoter or transcription initiation site selected from the group consisting of an inducible promoter, a CMV promoter or transcription initiation site, a T7 promoter or transcription initiation site, an EF1a promoter or transcription initiation site and combinations thereof.

In some embodiments, the first sequence encoding ORF1p and the second sequence encoding an endonuclease and/or a reverse transcriptase are codon optimized for expression in a human cell.

In some embodiments, the mRNA comprises a WPRE element. In some embodiments, the mRNA comprises a selection marker. In some embodiments, the mRNA comprises a sequence encoding an affinity tag. In some embodiments, the affinity tag is linked to the sequence encoding an endonuclease and/or a reverse transcriptase.

In some embodiments, the 3′ UTR comprises a poly A sequence or wherein a poly A sequence is added to the mRNA in vitro. In some embodiments, the poly A sequence is downstream of a sequence encoding an endonuclease and/or a reverse transcriptase. In some embodiments, the insert sequence is upstream of the poly A sequence.

In some embodiments, the 3′ UTR sequence comprises the insert sequence. In some embodiments, the insert sequence comprises a sequence that is a reverse complement of the sequence encoding the exogenous polypeptide. In some embodiments, the insert sequence comprises a polyadenylation site. In some embodiments, the insert sequence comprises an SV40 polyadenylation site. In some embodiments, the insert sequence comprises a polyadenylation site upstream of the sequence that is a reverse complement of the sequence encoding the exogenous polypeptide. In some embodiments, the insert sequence is integrated into the genome at a locus that is not a ribosomal locus. In some embodiments, the insert sequence integrates into a gene or regulatory region of a gene, thereby disrupting the gene or downregulating expression of the gene. In some embodiments, the insert sequence integrates into a gene or regulatory region of a gene, thereby upregulating expression of the gene. In some embodiments, the insert sequence integrates into the genome and replaces a gene. In some embodiments, the insert sequence is stably integrated into the genome. In some embodiments, the insert sequence is retrotransposed into the genome. In some embodiments, the insert sequence is integrated into the genome by cleavage of a DNA strand of a target site by an endonuclease encoded by the mRNA. In some embodiments, the insert sequence is integrated into the genome via target-primed reverse transcription (TPRT). In some embodiments, the insert sequence is integrated into the genome via reverse splicing of the mRNA into a DNA target site of the genome.

In some embodiments, the cell is an immune cell. In some embodiments, the immune cell is a T cell or a B cell. In some embodiments, the immune cell is a myeloid cell. In some embodiments, the immune cell is selected from a group consisting of a monocyte, a macrophage, a dendritic cell, a dendritic precursor cell, and a macrophage precursor cell.

In some embodiments, the mRNA is a self-integrating mRNA. In some embodiments, the method comprises introducing into the cell the mRNA. In some embodiments, the method comprises introducing into the cell the vector encoding the mRNA. In some embodiments, the method comprises introducing the mRNA or the vector encoding the mRNA into a cell ex vivo. In some embodiments, the method further comprises administering the cell to a human subject. In some embodiments, the method comprises administering the mRNA or the vector encoding the mRNA to a human subject. In some embodiments, an immune response is not elicited in the human subject. In some embodiments, the mRNA or the vector is substantially non-immunogenic.

In some embodiments, the vector is a plasmid or a viral vector. In some embodiments, the vector comprises a non-LTR retrotransposon. In some embodiments, the vector comprises a human L1 element.

In some embodiments, the vector comprises a L1 retrotransposon ORF1 gene. In some embodiments, the vector comprises a L1 retrotransposon ORF2 gene. In some embodiments, the vector comprises a L1 retrotransposon.

In some embodiments, the mRNA is at least about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 kilobases. In some embodiments, the mRNA is a most about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 kilobases.

In some embodiments, the mRNA comprises a payload that is at least about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 kilobases. In some embodiments, the mRNA is a most about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9 or 5 kilobases. In some embodiments, the mRNA is at least about 5.1, 5.2, 5.3, 5.4, 5.5, 5.6, 5.7, 5.8, 5.9 or 6 kilobases. In some embodiments, the mRNA is at least about 6.1, 6.2, 6.3, 6.4, 6.5, 6.6, 6.7, 6.8, 6.9 or 7 kilobases. In some embodiments, the mRNA is at least about 7.1, 7.2, 7.3, 7.4, 7.5, 7.6, 7.7, 7.8, 7.9 or 8 kilobases. In some embodiments, the mRNA is at least about 8.1, 8.2, 8.3, 8.4, 8.5, 8.6, 8.7, 8.8, 8.9 or 9 kilobases. In some embodiments, the mRNA is at least about 9.1, 9.2, 9.3, 9.4, 9.5, 9.6, 9.7, 9.8, 9.9 or 10 kilobases. In some embodiments, the mRNA is at least about 10.1, 10.2, 10.3, 10.4, 10.5, 10.6, 10.7, 10.8, 10.9 or 11 kilobases. In some embodiments, the mRNA is at least about 11.1, 11.2, 11.3, 11.4, 11.5, 11.6, 11.7, 11.8, 11.9 or 12 kilobases. In some embodiments, the mRNA comprises a payload of about 6.8 kB, e.g., a sequence encoding a ABCA4 gene product. In some embodiments, the mRNA comprises a payload of about 6.7 kB, e.g., a sequence encoding a MY07A gene product. In some embodiments, the mRNA comprises a payload of about 7.5 kB, e.g., a sequence encoding a CEP290 gene product. In some embodiments, the mRNA comprises a payload of about 10.1 kB, e.g., a sequence encoding a CDH23 gene product. In some embodiments, the mRNA comprises a payload of about 9.4 kB, e.g., a sequence encoding a EYS gene product. In some embodiments, the mRNA comprises a payload of about 15.6 kB, e.g., a sequence encoding a USH2a gene product. In some embodiments, the mRNA comprises a payload of about 12.5 kB, e.g., a sequence encoding a ALMS1 gene product. In some embodiments, the mRNA comprises a payload of about 4.6 kB, e.g., a sequence encoding a GDE gene product. In some embodiments, the mRNA comprises a payload of about 6 kB, e.g., a sequence encoding the OTOF gene product. In some embodiments, the mRNA comprises a payload of about 7.1 kB, e.g., a sequence encoding a F8 gene product.

One of the advantages of using the method of integration of a nucleic acid into the genome using retrotransposition is that it can be designed as described herein to deliver a nucleic acid cargo that is much larger than that using any other existing methods. For example, lentiviral and adeno-associated viral (AAV) gene delivery method are not expected to deliver a nucleic acid cargo of greater than 4 kB. In addition, lentiviral delivery entails risk of insertional mutagenesis and other toxicities. AAV mediated delivery entails unresolved liver and CNS toxicity. On the other hand, retrotransposition mediated method (Retro-T) using mRNA as described herein is rapid, safer and less complex than these viral methods.

In some embodiments, the mRNA comprises a sequence that inhibits or prevents degradation of the mRNA. In some embodiments, the sequence that inhibits or prevents degradation of the mRNA inhibits or prevents degradation of the mRNA by an exonuclease or an RNAse. In some embodiments, the sequence that inhibits or prevents degradation of the mRNA is a G quadruplex, pseudoknot or triplex sequence. In some embodiments, the sequence the sequence that inhibits or prevents degradation of the mRNA is an exoribonuclease-resistant RNA structure from a flavivirus RNA or an ENE element from KSV. In some embodiments, the sequence that inhibits or prevents degradation of the mRNA inhibits or prevents degradation of the mRNA by a deadenylase. In some embodiments, the sequence that inhibits or prevents degradation of the mRNA comprises non-adenosine nucleotides within or at a terminus of a poly A tail of the mRNA. In some embodiments, the sequence that inhibits or prevents degradation of the mRNA increases stability of the mRNA. In some embodiments, the exogenous sequence comprises a sequence encoding an exogenous polypeptide. In some embodiments, the sequence encoding an exogenous polypeptide is not in frame with a sequence encoding an endonuclease and/or a reverse transcriptase. In some embodiments, the sequence encoding an exogenous polypeptide is not in frame with a sequence encoding an endonuclease and/or a reverse transcriptase. In some embodiments, the exogenous sequence does not comprise introns. In some embodiments, the exogenous sequence comprises a sequence encoding an exogenous polypeptide selected from the group consisting of an enzyme, a receptor, a transport protein, a structural protein, a hormone, an antibody, a contractile protein and a storage protein. In some embodiments, the exogenous sequence comprises a sequence encoding an exogenous polypeptide selected from the group consisting of a chimeric antigen receptor (CAR), a ligand, an antibody, a receptor, and an enzyme. In some embodiments, the exogenous sequence comprises a regulatory sequence. In some embodiments, the regulatory sequence comprises a cis-acting regulatory sequence. In some embodiments, the regulatory sequence comprises a cis-acting regulatory sequence selected from the group consisting of an enhancer, a silencer, a promoter or a response element. In some embodiments, the regulatory sequence comprises a trans-acting regulatory sequence. In some embodiments, the regulatory sequence comprises a trans-acting regulatory sequence that encodes a transcription factor.

In some embodiments, integration of the insert sequence does not adversely affect cell health. In some embodiments, the endonuclease, the reverse transcriptase or both are capable of site-specific integration of the insert sequence.

In some embodiments, the mRNA comprises a sequence encoding an additional nuclease domain or a nuclease domain that is not derived from ORF2. In some embodiments, the mRNA comprises a sequence encoding a megaTAL nuclease domain, a TALEN domain, a Cas9 domain, a zinc finger binding domain from an R2 retroelement, or a DNA binding domain that binds to repetitive sequences such as a Rep78 from AAV. In some embodiments, the endonuclease comprises a mutation that reduces activity of the endonuclease compared to the endonuclease without the mutation. In some embodiments, the endonuclease is an ORF2p endonuclease and the mutation is S228P. In some embodiments, the mRNA comprises a sequence encoding a domain that increases fidelity and/or processivity of the reverse transcriptase. In some embodiments, the reverse transcriptase is a reverse transcriptase from a retroelement other than ORF2 or reverse transcriptase that has higher fidelity and/or processivity compared to a reverse transcriptase of ORF2p. In some embodiments, the reverse transcriptase is a group II intron reverse transcriptase. In some embodiments, the group II intron reverse transcriptase is a group IIA intron reverse transcriptase, a group IIB intron reverse transcriptase, or a group IIC intron reverse transcriptase. In some embodiments, the group II intron reverse transcriptase is TGIRT-II or TGIRT-III.

In some embodiments, the mRNA comprises a sequence comprising an Alu element and/or a ribosome binding aptamer. In some embodiments, the mRNA comprises a sequence encoding a polypeptide comprising a DNA binding domain. In some embodiments, the 3′ UTR sequence is derived from a viral 3′ UTR or a beta-globin 3′ UTR.

In one aspect, provided herein is a composition comprising a recombinant mRNA or vector encoding an mRNA, wherein the mRNA comprises a human LINE-1 transposon sequence comprising a human LINE-1 transposon 5′ UTR sequence, a sequence encoding ORF1p downstream of the human LINE-1 transposon 5′ UTR sequence, an inter-ORF linker sequence downstream of the sequence encoding ORF1p,a sequence encoding ORF2p downstream of the inter-ORF linker sequence, and a 3′ UTR sequence derived from a human LINE-1 transposon downstream of the sequence encoding ORF2p; wherein the 3′ UTR sequence comprises an insert sequence, wherein the insert sequence is a reverse complement of a sequence encoding an exogenous polypeptide or a reverse complement of a sequence encoding an exogenous regulatory element.

In some embodiments, the insert sequence integrates into the genome of a cell when introduced into the cell. In some embodiments, the insert sequence integrates into a gene associated a condition or disease, thereby disrupting the gene or downregulating expression of the gene. In some embodiments, the insert sequence integrates into a gene, thereby upregulating expression of the gene. In some embodiments, the recombinant mRNA or vector encoding the mRNA is isolated or purified.

In one aspect, provided herein is a composition comprising a nucleic acid comprising a nucleotide sequence encoding (a) a long interspersed nuclear element (LINE) polypeptide, wherein the LINE polypeptide includes human ORF1p and human ORF2p; and (b) an insert sequence, wherein the insert sequence is a reverse complement of a sequence encoding an exogenous polypeptide or a reverse complement of a sequence encoding an exogenous regulatory element, wherein the composition is substantially non-immunogenic.

In some embodiments, the composition comprises human ORF1p and human ORF2p proteins. In some embodiments, the composition comprises a ribonucleoprotein (RNP) comprising human ORF1p and human ORF2p complexed to the nucleic acid. In some embodiments, the nucleic acid is mRNA.

In one aspect, provided herein is a composition comprising a cell comprising a composition described herein. In some embodiments, the cell is an immune cell. In some embodiments, the immune cell is a T cell or a B cell. In some embodiments, the immune cell is a myeloid cell. In some embodiments, the immune cell is selected from a group consisting of a monocyte, a macrophage, a dendritic cell, a dendritic precursor cell, and a macrophage precursor cell. In some embodiments, the insert sequence is a reverse complement of a sequence encoding an exogenous polypeptide and the exogenous polypeptide is a chimeric antigen receptor (CAR).

In one aspect, provided herein is a pharmaceutical composition comprising a composition described herein, and a pharmaceutically acceptable excipient. In some embodiments, the pharmaceutical composition is for use in gene therapy. In some embodiments, the pharmaceutical composition is for use in the manufacture of a medicament for treating a disease or condition. In some embodiments, the pharmaceutical composition is for use in treating a disease or condition. In one aspect, provided herein is a method of treating a disease in a subject, comprising administering a pharmaceutical composition described herein to a subject with a disease or condition. In some embodiments, the method increases an amount or activity of a protein or functional RNA in the subject. In some embodiments, the subject has a deficient amount or activity of a protein or functional RNA. In some embodiments, the deficient amount or activity of a protein or functional RNA is associated with or causes the disease or condition.

In some embodiments, the method further comprising administering an agent that inhibits human silencing hub (HUSH) complex, an agent that inhibits FAM208A, or an agent that inhibits TRIM28. In some embodiments, the agent that inhibits human silencing hub (HUSH) complex is an agent that inhibits Periphilin, TASOR and/or MPP8. In some embodiments, the agent that inhibits human silencing hub (HUSH) complex inhibits assembly of the HUSH complex.

In some embodiments, the agent inhibits the Fanconi anemia complex. In some embodiments, the agent inhibits FANCD2-FANC1 heterodimer monoubiquitylation. In some embodiments, the agent inhibits FANCD2-FANC1 heterodimer formation. In some embodiments the agent inhibits the Fanconi Anemia (FA) core complex. FA core complex is a component of the Fanconi anemia DNA damage repair pathway, e.g., in chemotherapy induced DNA inter-strand crosslinks. The FA core complex comprises two central dimers of the FANCB and FA-associated protein of 100 kDa (FAAPI00) subunits, flanked by two copies of the RING finger subunit, FANCL. These two heterotrimers act as a scaffold to assemble the remaining five subunits, resulting in an extended asymmetric structure. Destabilization of the scaffold would disrupt the entire complex, resulting in a non-functional FA pathway. Examples of agents that can inhibit the FA core complex include Bortezomib and curcumin analogs EF24 and 4l-TTD.

In some embodiments, the sequences to be inserted may be placed under the control of tissue-specific elements, such that the entire inserted DNA is only functional in those cells in which the tissue-specific element is active.

In one aspect, provided herein are method and compositions for stable gene transfer to a cell by introducing to the cell a heterologous nucleic acid or gene of interest (e.g., a transgene, a regulatory sequence, for example, a sequence for an inhibitory nucleic acid, an siRNA, a miRNA), flanked by sequences that cause retrotransposition of the heterologous nucleic acid sequence into the genome of the cell. In some embodiments, the heterologous nucleic acid is termed insert for the purpose of the description in this document, where the insert is the nucleic acid sequence that will be reverse transcribed and inserted into the genome of the cell by the intended design of the constructs described herein. In some embodiments, the heterologous nucleic acid is also termed the cargo, or cargo sequence for the purpose of the description in this document. The cargo can comprise the sequence of the heterologous nucleic acid that that is inserted in the genome. In some embodiments, the cell may be a cell mammalian cell. The mammalian cell may be of epithelial, mesothelial or endothelial origin. In some embodiments, the cell may be a stem cell. In some embodiments, the cell may be a precursor cell. In some embodiments, the cell may be a cell that is terminally differentiated. In some embodiments, the cell may be a muscle cell, a cardiac cell, an epithelial cell, a hematopoietic cell, a mucous cell, an epidermal cell, a squamous cell, a cartilage cell, a bone cell, or any cell of mammalian origin. In some embodiments, the cell is of hematopoietic lineage. In some embodiments, he cell is of myeloid lineage, or a phagocytic cell, for example a monocyte, macrophage, a dendritic cell or a myeloid precursor cell. In some embodiments, the nucleic acid encoding the transgene is an mRNA.

In some embodiments, the retrotransposable elements may be derived from a non-LTR retrotransposon.

Provided herein is a method of integrating a nucleic acid sequence into a genome of a cell, the method comprising introducing a recombinant mRNA or a vector encoding an mRNA into the cell, wherein the mRNA comprises an insert sequence and wherein the insert sequence is integrated into the genome of the cell. In some embodiments, the insert sequence comprises (i) an exogenous sequence, or (ii) a sequence that is a reverse complement of the exogenous sequence; a 5′ UTR sequence and a 3′ UTR sequence downstream of the 5′ UTR sequence; wherein the 5′ UTR sequence or the 3′ UTR sequence comprises a binding site for a human ORF protein. In some embodiments, the ORF protein is a human LINE 1 ORF2 protein. In some embodiments, the ORF protein is a non-human ORF protein. In some embodiments, the ORF protein is a chimeric protein, a recombinant protein or an engineered protein.

Provided herein is a method for integrating a nucleic acid sequence into the genome of an immune cell, the method comprising introducing a recombinant mRNA or a vector encoding an mRNA, wherein the mRNA comprises, (a) an insert sequence, wherein the insert sequence comprises (i) an exogenous sequence or (ii) a sequence that is a reverse complement of the exogenous sequence; (b) 5′ UTR sequence and a 3′ UTR sequence downstream of the 5′ UTR sequence, wherein the 5′ UTR sequence or the 3′ UTR sequence comprises an endonuclease binding site and a reverse transcriptase binding site, and wherein the transgene sequence is integrated into the genome of the immune cell.

In some embodiments, the structural elements that mediate RNA integration or transposition may be encoded in a synthetic construct and are relied upon to deliver a heterologous gene of interest to the cell.

In some embodiments, the synthetic construct may comprise a nucleic acid encoding the heterologous gene of interest and the structural elements that cause integration or retrotransposition of a heterologous gene of interest into the genome. In some embodiments, the structural elements that cause integration or retrotransposition may include a 5′ L1 RNA region, and a 3′-L1 region, the latter comprising a poly A 3′ region for priming. In some embodiments, the 5′ L1 RNA region may comprise one or more stem loop regions. In some embodiments, the L1-3′ region may comprise one or more stem loop regions. In some embodiments, the 5′- and 3′ L1 regions are constructed as flanking the nucleic acid sequence encoding the heterologous gene of interest (the transgene). In some embodiments, the structural elements may include a region from an L1 or an Alu RNA comprising the hairpin loop structure that includes the A-Box and the B-Box elements that are ribosomal binding sites In some embodiments, the synthetic nucleic acid may comprise a L1-Ta promoter.

There may be two types of LINE RNA recognition by ORF2p—the stringent and the relaxed. In the stringent type RT recognizes its own 3′UTR tail, and in the relaxed type RT does not require any specific recognition except for the poly-A tail. Division into the stringent and the relaxed type came from the observation that some LINE/SINE pairs share the same 3′-end. For the stringent type, the experimental studies showed that a 3′UTR stem-loop promotes retrotransposition. The 5′-UTR of the LINE retrotransposition sequences have been shown to contain three conserved stem loop regions.

In some embodiments, the transgene, or transcript of interest may be flanked by transposable elements from a L1 or an Alu sequence at the 5′ and the 3′ end. In some embodiments, the 5′ region of a retrotransposon comprises an Alu sequence. In some embodiments, the 3′ region of a retrotransposon comprises an Alu sequence. In some embodiments, the 5′ region of a retrotransposon comprises an L1 sequence. In some embodiments, the 3′ region of a retrotransposon comprises an L1 sequence. In some embodiments, the transgene or transcript of interest is flanked by an SVA transposon sequence.

In some embodiments, the transcript of interest may comprise an L1 or an Alu sequence, encoding the binding regions for ORF2p and the 3′—poly A priming regions. In some embodiments, the heterologous nucleic acid encoding the transgene of interest may be flanked by an L1 or an Alu sequence, encoding the binding regions for ORF1p and the 3′—poly A priming regions. The 3′-region may comprise one or more stem loop structures. In some embodiments, the transcript of interest is structured for cis integration or retrotransposition. In some embodiments, the transcript of interest is structured for trans integration or retrotransposition.

In some embodiments, the retrotransposon is a human retrotransposon. The sequence of a human retrotransposon can comprise a sequence encoding an endonuclease and/or a reverse transcriptase. The sequence of a human retrotransposon can encode for two proteins that are translated from a single RNA containing two non-overlapping ORFs. In some embodiments, the two ORFs are ORF1 and ORF2.

Accordingly, provided herein is a method for stably integrating a heterologous nucleic acid encoding a transgene into the genome of a cell, such as a myeloid cell, the method comprising introducing to the cell a nucleic acid encoding: the transgene; one or more 5′nucleic acid sequences flanking the region encoding the transgene, comprising a 5′ region of a retrotransposon; and one or more 3′ nucleic acid sequence flanking the region encoding the transgene, comprising a 3′ region of a retrotransposon, wherein the 3′ region of the retrotransposon comprises a genomic DNA priming sequence and a LINE transposase binding sequence, having the respective endonuclease and reverse transcriptase (RT) activity.

Provided herein is a method for integrating a nucleic acid sequence into the genome of a cell, the method comprising introducing a recombinant mRNA or a vector encoding an mRNA, wherein the mRNA comprises an insert sequence, wherein the insert sequence comprises (i) an exogenous sequence or (ii) a sequence that is a reverse complement of the exogenous sequence; (b) a 5′ UTR sequence, a sequence of a human retrotransposon downstream of the 5′ UTR sequence, and a 3′ UTR sequence downstream of the sequence of a human retrotransposon; wherein the 5′ UTR sequence or the 3′ UTR sequence comprises an endonuclease binding site and a reverse transcriptase binding site, and wherein the sequence of a human retrotransposon encodes for two proteins that are translated from a single RNA containing two ORFs, and wherein the insert sequence is integrated into the genome of the cell.

In some embodiments, the method comprising using a single nucleic acid molecule for delivering and integrating the insert sequence into the genome of a cell. The single nucleic acid molecule may be a plasmid vector. The single nucleic acid may be DNA or an RNA molecule. The single nucleic acid may be an mRNA.

In some embodiments, the method comprises introducing into a cell one or more polynucleotides comprising the human retrotransposon and a heterologous nucleic acid sequence. In some embodiments, the one or more polynucleotides comprises (i) a first nucleic acid molecule encoding an ORF1p; (ii) a second nucleic acid molecule encoding an ORF2p and a sequence encoding a cargo. In some embodiments, the first nucleic acid and the second nucleic acid are mRNA. In some embodiments, the first nucleic acid and the second nucleic acid are DNA, e.g., encoded in separate plasmid vectors.

Provided herein is a self-integrating polynucleotide that comprises a sequence which is inserted into the genome of a cell, and insert is stably integrated into the genome by the self-integrating naked polynucleotide. In some embodiments, the polynucleotide is an RNA. In some embodiments, the polynucleotide is an mRNA. In some embodiments, the polynucleotide is an mRNA that has modifications.

In some embodiments, the modifications ensure protection against RNases in the intracellular milieu. In some embodiments, the modifications include substituted modified nucleotides, e.g., 5-methylcytidine, pseudouridine or 2-thiouridine.

In some embodiments, a single polynucleotide is used for delivery and genomic integration of the insert (or cargo) nucleic acid. In some embodiments, the single polynucleotide is bicistronic. In some embodiments, the single polynucleotide is tricistronic. In some embodiments, the single polynucleotide is multi-cistronic. In some embodiments, a two or more polynucleotide molecules are used for delivery and genomic integration of the insert (or cargo) nucleic acid.

In some embodiments, a retrotransposable genetic element may be generated, the retrotransposable genetic element comprising (i) a heterologous nucleic acid encoding a transgene or a non-coding sequence to be inserted into the genome of a cell (the insert); (ii) a nucleic sequence encoding one or more retrotransposon ORF-encoding sequences; (iii) one or more UTR regions of the ORF-coding sequences, such that the heterologous nucleic acid encoding a transgene or a non-coding sequence to be inserted is comprised within the UTR sequences; wherein the 3′ region of the retrotransposon ORF-encoding sequences comprises a genomic DNA priming sequence.

In some embodiments, the retrotransposable genetic element may be introduced into a cell for stably integrating the transgene into the genomic DNA. In some embodiments, the retrotransposable genetic element comprises (a) a retrotransposon protein coding sequence, and a 3′ UTR; and (b) a sequence comprising a heterologous nucleic acid that is to be inserted (e.g., integrated) within the genome of a cell.

The retrotransposon protein coding sequence, and the 3′ UTR may be a complete and sufficient unit for delivering the heterologous nucleic acid sequence within the genome of the cell, and comprise the retrotransposable elements, such as an endonuclease, a reverse transcriptase, a sequence in the 3′ UTR for binding to and priming the genomic DNA at the region cleaved by the endonuclease to start reverse transcribing and incorporating the heterologous nucleic acid.

In some embodiments, the coding sequence of the insert is in forward orientation with respect to the coding sequence of the one or more ORFs. In some embodiments, the coding sequence of the insert is in reverse orientation with respect to the coding sequence of the one or more ORFs. The coding sequence of the insert and the coding sequence of the one or more ORFs may comprise distinct regulatory elements, including 5′ UTR, 3′ UTR, promoter, enhancer, etc. In some embodiments, the 3′ UTR or the 5′-UTR of the insert may comprise the coding sequence of the one or more ORFs, and likewise, the coding sequence of the insert may be situated within in the 3′ UTR of the coding sequence of the one or more ORFs.

In some embodiments, a retrotransposable genetic element may be generated, the retrotransposable genetic element comprising: (a) an insert sequence, comprising (i) an exogenous sequence, a sequence that is a reverse complement of the exogenous sequence; a 5′ UTR sequence and a 3′ UTR sequence downstream of the 5′ UTR sequence; wherein the 5′ UTR sequence or the 3′ UTR sequence comprises a binding site for a human ORF protein.

In some embodiments, the retrotransposon may comprise a SINE or LINE element. In some embodiments, the retrotransposon comprises a SINE or LINE stem loop structure, such as an Alu element.

In some embodiments, the retrotransposon is a LINE-1 (L1) retrotransposon. In some embodiments, the retrotransposon is human LINE-1. Human LINE-1 sequences are abundant in the human genome. There are approximately 13,224 total human L1s, of which 480 are active, which make up about 3.6%. Therefore, human L1 proteins are well tolerated and non-immunogenic in humans. Moreover, a tight regulation of random transposition in human ensures that random transposase activity will not be triggered by introduction of the L1 system as described herein. In addition, the retrotransposable constructs designed herein may comprise targeted and specific incorporation of the insert sequence. In some embodiments, the retrotransposable genetic element may comprise designs intended to overcome the silencing machinery actively prevalent in human cells, while being careful that random integration resulting in genomic instability is not initiated.

Accordingly, the retrotransposable constructs may comprise a sequence encoding a human LINE-1 ORF1 protein; and a human LINE-1 ORF2 protein. In some embodiments, the construct comprises a nucleic acid sequence encoding an ORF1p protein with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to MGKKQNRKTGNSKTQSASPPPKERSSSPATEQSWMENDFDELREEGFRRSNYSELREDIQTKGK EVENFEKNLEECITRITNTEKCLKELMELKTKARELREECRSLRSRCDQLEERVSAMEDEMNEM KREGKFREKRIKRNEQSLQEIWDYVKRPNLRLIGVPESDVENGTKLENTLQDIIQENFPNLARQA NVQIQEIQRTPQRYSSRRATPRHIIVRFTKVEMKEKMLRAAREKGRVTLKGKPIRLTVDLSAETL QARREWGPIFNILKEKNFQPRISYPAKLSFISEGEIKYFIDKQMLRDFVTTRPALKELLKEALNME RNNRYQPLQNHAKM (SEQ ID NO: 57). In some embodiments, the construct comprises a nucleic acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to

(SEQ ID NO: 58)
atgggcaagaagcaaaatcgcaagacggggaattc
caagacacaatccgctagcccaccacctaaagagc
gttctagctcccctgctactgagcagtcctggatg
gaaaacgacttcgatgaactccgggaagagggatt
taggcgatccaactattcagaactccgcgaagata
tccagacaaaggggaaggaagtcgagaatttcgag
aagaacctcgaggagtgcatcacccgtatcacaaa
cactgagaaatgtctcaaagaactcatggaactta
agacaaaagccagggagcttcgagaggagtgtcgg
agtctgagatccaggtgtgaccagctcgaggagcg
cgtgagcgcgatggaagacgagatgaacgagatga
aaagagagggcaaattcagggagaagcgcattaag
aggaacgaacagagtctgcaggagatttgggatta
cgtcaagaggcctaacctgcggttgatcggcgtcc
ccgagagcgacgtagaaaacgggactaaactggag
aatacacttcaagacatcattcaagaaaattttcc
aaacctggctcggcaagctaatgtgcaaatccaag
agatccaacgcacaccccagcggtatagctctcgg
cgtgccacccctaggcatattatcgtgcgctttac
taaggtggagatgaaagagaagatgctgcgagccg
ctcgggaaaagggaagggtgactttgaagggcaaa
cctattcggctgacggttgaccttagcgccgagac
actccaggcacgccgggaatggggccccatcttta
atatcctgaaggagaagaacttccagccacgaatc
tcttaccctgcaaagttgagttttatctccgaggg
tgagattaagtatttcatcgataaacagatgctgc
gagacttcgtgacaactcgcccagctctcaaggaa
ctgctcaaagaggctcttaatatggagcgcaataa
tagatatcaacccttgcagaaccacgcaaagatg
tga.

In some embodiments, the construct comprises a nucleic acid sequence encoding an ORF2p protein with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to MTGSNSHITILTLNINGLNSAIKRHRLASWIKSQDPSVCCIQETHLTCRDTHRLKIKGWRKIYQAN GKQKKAGVAILVSDKTDFKPTKIKRDKEGHYIMVKGSIQQEELTILNIYAPNTGAPRFIKQVLSDL QRDLDSHTLIMGDFNTPLSTLDRSTRQKVNKDTQELNSALHQADLIDIYRTLHPKSTEYTFFSAP HHTYSKIDHIVGSKALLSKCKRTEIITNYLSDHSAIKLELRIKNLTQSRSTTWKLNNLLLNDYWV HNEMKAEIKMFFETNENKDTTYQNLWDAFKAVCRGKFIALNAYKRKQERSKIDTLTSQLKELE KQEQTHSKASRRQEITKIRAELKEIETQKTLQKINESRSWFFERINKIDRPLARLIKKKREKNQIDTI KNDKGDITTDPTEIQTTIREYYKHLYANKLENLEEMDTFLDTYTLPRLNQEEVESLNRPITGSEIV AIINSLPTKKSPGPDGFTAEFYQRYMEELVPFLLKLFQSIEKEGILPNSFYEASIILIPKPGRDTTKKE NFRPISLMNIDAKILNKILANRIQQHIKKLIHHDQVGFIPGMQGWFNIRKSINVIQHINRAKDKNH MIISIDAEKAFDKIQQPFMLKTLNKLGIDGTYFKIIRAIYDKPTANIILNGQKLEAFPLKTGTRQGC PLSPLLFNIVLEVLARAIRQEKEIKGIQLGKEEVKLSLFADDMIVYLENPIVSAQNLLKLISNFSKV SGYKINVQKSQAFLYTNNRQTESQIMGELPFVIASKRIKYLGIQLTRDVKDLFKENYKPLLKEIKE DTNKWKNIPCSWVGRINIVKMAILPKVIYRFNAIPIKLPMTFFTELEKTTLKFIWNQKRARIAKSIL SQKNKAGGITLPDFKLYYKATVTKTAWYWYQNRDIDQWNRTEPSEIMPHIYNYLIFDKPEKNK QWGKDSLFNKWCWENWLAICRKLKLDPFLTPYTKINSRWIKDLNVKPKTIKTLEENLGITIQDIG VGKDFMSKTPKAMATKDKIDKWDLIKLKSFCTAKETTIRVNRQPTTWEKIFATYSSDKGLISRIY NELKQIYKKKTNNPIKKWAKDMNRHFSKEDIYAAKKHMKKCSSSLAIREMQIKTTMRYHLTPV RMAIIKKSGNNRCWRGCGEIGTLLHCWWDCKLVQPLWKSVWRFLRDLELEIPFDPAIPLLGIYP NEYKSCCYKDTCTRMFIAALFTIAKTWNQPKCPTMIDWIKKMWHIYTMEYYAAIKNDEFISFVG TWMKLETIILSKLSQEQKTKHRIFSLIGGN (SEQ ID NO: 59). In some embodiments, the construct comprises a nucleic acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to

(SEQ ID NO: 60)
atgaccggctctaactcacatatcaccatccttac
acttaacattaacggcctcaactcagctatcaagc
gccatcggctggccagctggatcaaatcacaggat
ccaagcgtttgttgcatccaagagacccacctgac
ctgtagagatactcaccgcctcaagatcaagggat
ggcgaaagatttatcaggcgaacggtaagcagaag
aaagccggagtcgcaattctggtctcagacaagac
ggatttcaagcccaccaaaattaagcgtgataagg
aaggtcactatattatggtgaaaggcagcatacag
caggaagaacttaccatattgaacatctacgcgcc
aaacaccggcgcacctcgctttatcaaacaggtcc
tgtccgatctgcagcgagatctggattctcatacg
ttgattatgggtgatttcaatacaccattgagcac
cctggatcgcagcaccaggcaaaaggtaaataaag
acacgcaagagctcaatagcgcactgcatcaggca
gatctcattgatatttatcgcactcttcatcctaa
gagtaccgagtacacattcttcagcgccccacatc
atacatactcaaagatcgatcatatcgtcggctca
aaggctctgctgtcaaagtgcaagcgcacagagat
aattacaaattacctgtcagatcatagcgcgatca
agctcgagctgagaatcaagaacctgacccagagc
cggagtaccacttggaagcttaataacctgctgct
caacgattattgggtccacaatgagatgaaggcag
agattaaaatgttcttcgaaacaaatgagaataag
gatactacctatcaaaacctttgggatgcctttaa
ggccgtctgcagaggcaagttcatcgccctcaacg
cctataaaagaaaacaagagagatctaagatcgat
actctcacctctcagctgaaggagttggagaaaca
ggaacagacccactccaaggcgtcaagacggcagg
agatcacaaagattcgcgccgagttgaaagagatc
gaaacccaaaagactcttcagaaaattaacgagtc
tcgtagttggttcttcgagcggattaataagatag
acagacctctggcacgactgattaagaagaagcgc
gaaaagaaccagattgataccatcaagaacgacaa
gggcgacatcactactgacccgaccgagatccaga
ccactattcgggagtattataagcatttgtatgct
aacaagcttgagaacctggaagagatggacacttt
tctggatacctatactctgccacggcttaatcaag
aggaagtcgagtccctcaaccgcccaattacagga
agcgagattgtggccataattaactccctgccgac
aaagaaatctcctggtccggacgggtttacagctg
agttttatcaacggtatatggaagagcttgtaccg
tttctgctcaagctctttcagtctatagaaaagga
aggcatcttgcccaattccttctacgaagcttcta
taatacttattcccaaaccaggacgcgataccaca
aagaaggaaaacttccggcccattagtctcatgaa
tatcgacgctaaaatattgaacaagattctcgcca
acagaatccaacaacatattaagaaattgatacat
cacgaccaggtggggtttatacctggcatgcaggg
ctggtttaacatccggaagagtattaacgtcattc
aacacattaatagagctaaggataagaatcatatg
atcatctctatagacgcggaaaaggcattcgataa
gattcagcagccatttatgctcaagactctgaaca
aactcggcatcgacggaacatattttaagattatt
cgcgcaatttacgataagccgactgctaacattat
ccttaacggccaaaagctcgaggcctttccgctca
agactggaacccgccaaggctgtcccctctccccg
cttttgtttaatattgtactcgaggtgctggctag
ggctattcgtcaagagaaagagattaaagggatac
agctcgggaaggaagaggtcaagctttccttgttc
gccgatgatatgattgtgtacctggagaatcctat
tgtgtctgctcagaaccttcttaaacttatttcta
actttagcaaggtcagcggctataagattaacgtc
cagaaatctcaggcctttctgtacacaaataatcg
acagaccgaatcccagataatgggtgagcttccgt
ttgtcatagccagcaaaaggataaagtatctcgga
atccagctgacacgagacgttaaagatttgtttaa
ggaaaattacaagcctctcctgaaagagattaagg
aagatactaataagtggaagaatatcccctgttca
tgggttggcagaatcaacatagtgaagatggcaat
acttcctaaagtgatatatcgctttaacgccatcc
caattaaactgcctatgaccttctttacggagctc
gagaaaacaacccttaaatttatatggaatcaaaa
gagagcaagaatagcgaagtccatcttgagccaga
agaataaggccggtgggattactttgcctgatttt
aagttgtattataaagccacagtaactaagacagc
ctggtattggtatcagaatagagacatcgaccagt
ggaatcggaccgaaccatcagagataatgccccac
atctataattaccttatattcgataagccagaaaa
gaataaacagtggggcaaagacagcctcttcaaca
agtggtgttgggagaattggctggccatatgccgg
aaactcaagctcgacccctttcttacaccctacac
taaaatcaacagtaggtggatcaaggacttgaatg
tcaagccaaagactataaagacactggaagagaat
cttgggatcacaatacaagatataggcgtcggcaa
agattttatgtcaaagacgcccaaggccatggcca
ctaaggataagattgataagtgggaccttattaag
ctcaaaagcttctgtactgccaaggagaccacgat
cagagttaataggcagcccactacatgggaaaaga
ttttcgccacttattcatcagataaggggttgata
agcagaatatataacgagctgaagcagatctacaa
gaagaaaacgaataatcccatcaagaagtgggcaa
aagatatgaacaggcattttagcaaagaggatatc
tacgccgcgaagaagcatatgaagaagtgtagttc
aagcttggccattcgtgagatgcagattaagacga
ccatgcgataccaccttaccccagtgaggatggca
attatcaagaaatctggcaataatagatgttggcg
gggctgtggcgagattggcaccctgctccattgct
ggtgggattgcaagctggtgcagccgctttggaaa
tcagtctggcgctttctgagggacctcgagcttga
gattcccttcgatcccgcaattcccttgctcggaa
tctatcctaacgaatacaagagctgttgttacaag
gatacgtgtacccggatgttcatcgcggccttgtt
tacgatagctaagacgtggaatcagcctaagtgcc
ccacaatgatcgattggatcaagaaaatgtggcat
atttataccatggagtattacgcagcaattaagaa
tgacgaatttatttccttcgttgggacctggatga
agctggagactattattctgagcaagctgtctcag
gagcaaaagacaaagcatagaatcttctctctcat
tggtggtaactaa.

In some embodiments, the construct comprises a nucleic acid sequence encoding an ORF2p protein with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to MVIGTYISIITLNVNGLNAPTKRHRLAEWIQKQDPYICCLQETHFRPRDTYRLKVRGWKKIFHAN GNQKKAGVAILISDKIDFKIKNVTRDKEGHYIMIQGSIQEEDITIINIYAPNIGAPQYIRQLLTAIKE EIDSNTIIVGDFNTSLTPMDRSSKMKINKETEALNDTIDQIDLIDIYRTFHPKTADYTFFSSAHGTFS RIDHILGHKSSLSKFKKIEIISSIFSDHNAMRLEMNHREKNVKKTNTWRLNNTLLNNQEITEEIKQ EIKKYLETNDNENTTTQNLWDAAKAVLRGKFIAIQAYLKKQEKSQVNNLTLHLKKLEKEEQTK PKVSRRKEIIKIRAEINEIETKKTIAKINKTKSWFFEKINKIDKPLARLIKKKRERTQINKIRNEKGE VTTDTAEIQNILRDYYKQLYANKMDNLEEMDKFLERYNLPRLNQEETENINRPITSNEIETVIKNL PTNKSPGPDGFTGEFYQTFREELTPILLKLFQKIAEEGTLPNSFYEATITLIPKPDKDTTKKENYRPI SLMNIDAKILNKILANRIQQHIKRIIHHDQVGFIPGMQGFFNIRKSINVIHHINKLKKKNHMIISIDA EKAFDKIQHPFMIKTLQKVGIEGTYLNIIKAIYDKPTANIILNGEKLKAFPLRSGTRQGCPLSPLLF NIVLEVLATAIREEKEIKGIQIGKEEVKLSLFADDMILYIENPKTATRKLLELINEYGKVAGYKINA QKSLAFLYTNDEKSEREIMETLPFTIATKRIKYLGINLPKETKDLYAENYKTLMKEIKDDTNRWR DIPCSWIGRINIVKMSILPKAIYRFNAIPIKLPMAFFTELEQIILKFVWRHKRPRIAKAVLRQKNGA GGIRLPDFRLYYKATVIKTIWYWHKNRNIDQWNKIESPEINPRTYGQLIYDKGGKDIQWRKDSLF NKWCWENWTATCKRMKLEYSLTPYTKINSKWIRDLNIRLDTIKLLEENIGRTLFDINHSKIFFDPP PRVMEIKTKINKWDLMKLQSFCTAKETINKTKRQPSEWEKIFANESTDKGLISKIYKQLIQLNIKE TNTPIQKWAEDLNRHFSKEDIQTATKHMKRCSTSLIIREMQIKTTMRYHLTPVRMGIIRKSTNNK CWRGCGEKGTLLHCWWECKLIQPLWRTIWRFLKKLKIELPYDPAIPLLGIYPEKTVIQKDTCTR MFIAALFTIARSWKQPKCPSTDEWIKKMWYIYTMEYYSAIKRNEIGSFLETWMDLETVIQSEVSQ KEKNKYRILTHICGTWKNGTDEPVCRTEIETQM (SEQ ID NO: 61). In some embodiments, the construct comprises a nucleic acid sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to

(SEQ ID NO: 62)
atggtcataggaacatacatatcgataattacctt
aaacgtgaatggattaaatgccccaaccaaaagac
atagactggctgaatggatacaaaaacaagaccca
tatatatgctgtctacaagagacccacttcagacc
tagggacacatacagactgaaagtgaggggatgga
aaaagatattccatgcaaatggaaatcaaaagaaa
gctggagtagctatactcatatcagataaaataga
ctttaaaataaagaatgttacaagagacaaggaag
gacactacataatgatccagggatcaatccaagaa
gaagatataacaattataaatatatatgcacccaa
cataggagcacctcaatacataaggcaactgctaa
cagctataaaagaggaaatcgacagtaacacaata
atagtgggggactttaacacctcacttacaccaat
ggacagatcatccaaaatgaaaataaataaggaaa
cagaagctttaaatgacacaatagaccagatagat
ttaattgatatatataggacattccatccaaaaac
agcagattacacgttcttctcaagtgcgcacggaa
cattctccaggatagatcacatcttgggtcacaaa
tcaagcctcagtaaatttaagaaaattgaaatcat
atcaagcatcttttctgaccacaacgctatgagat
tagaaatgaatcacagggaaaaaaacgtaaaaaag
acaaacacatggaggctaaacaatacgttactaaa
taaccaagagatcactgaagaaatcaaacaggaaa
taaaaaaatacctagagacaaatgacaatgaaaac
acgacgacccaaaacctatgggatgcagcaaaagc
ggttctaagagggaagtttatagctatacaagcct
acctaaagaaacaagaaaaatctcaagtaaacaat
ctaaccttacacctaaagaaactagagaaagaaga
acaaacaaaacccaaagttagcagaaggaaagaaa
tcataaagatcagagcagaaataaatgaaatagaa
acaaagaaaacaatagcaaagatcaataaaactaa
aagttggttctttgagaagataaacaaaattgata
agccattagccagactcatcaagaaaaagagggag
aggactcaaatcaataaaatcagaaatgaaaaagg
agaagttacaacagacaccgcagaaatacaaaaca
tcctaagagactactacaagcaactttatgccaat
aaaatggacaacctggaagaaatggacaaattctt
agaaaggtataaccttccaagactgaaccaggaag
aaacagaaaatatcaacagaccaatcacaagtaat
gaaattgaaactgtgattaaaaatcttccaacaaa
caaaagtccaggaccagatggcttcacaggtgaat
tctatcaaacatttagagaagagctaacacccatc
cttctcaaactcttccaaaaaattgcagaagaagg
aacactcccaaactcattctatgaggccaccatca
ccctgataccaaaaccagacaaagacactacaaaa
aaagaaaattacagaccaatatcactgatgaatat
agatgcaaaaatcctcaacaaaatactagcaaaca
gaatccaacaacacattaaaaggatcatacaccac
gatcaagtgggatttatcccagggatgcaaggatt
cttcaatatacgcaaatcaatcaatgtgatacacc
atattaacaaattgaagaagaaaaaccatatgatc
atctcaatagatgcagaaaaagcttttgacaaaat
tcaacacccatttatgataaaaactctccagaaag
tgggcatagagggaacctacctcaacataataaag
gccatatatgacaaacccacagcaaacatcattct
caatggtgaaaaactgaaagcatttcctctaagat
caggaacgagacaaggatgtccactctcaccacta
ttattcaacatagttctggaagtcctagccacggc
aatcagagaagaaaaagaaataaaaggaatacaaa
ttggaaaagaagaagtaaaactgtcactgtttgcg
gatgacatgatactatacatagagaatcctaaaac
tgccaccagaaaactgctagagctaattaatgaat
atggtaaagttgcaggttacaaaattaatgcacag
aaatctcttgcattcctatacactaatgatgaaaa
atctgaaagagaaattatggaaacactcccattta
ccattgcaacaaaaagaataaaatacctaggaata
aacctacctaaggagacaaaagacctgtatgcaga
aaactataagacactgatgaaagaaattaaagatg
ataccaacagatggagagatataccatgttcttgg
attggaagaatcaacattgtgaaaatgagtatact
acccaaagcaatctacagattcaatgcaatcccta
tcaaattaccaatggcattttttacggagctagaa
caaatcatcttaaaatttgtatggagacacaaaag
accccgaatagccaaagcagtcttgaggcaaaaaa
atggagctggaggaatcagactccctgacttcaga
ctatactacaaagctacagtaatcaagacaatatg
gtactggcacaaaaacagaaacatagatcaatgga
acaagatagaaagcccagagattaacccacgcacc
tatggtcaactaatctatgacaaaggaggcaaaga
tatacaatggagaaaagacagtctcttcaataagt
ggtgctgggaaaactggacagccacatgtaaaaga
atgaaattagaatactccctaacaccatacacaaa
aataaactcaaaatggattagagacctaaatataa
gactggacactataaaactcttagaggaaaacata
ggaagaacactctttgacataaatcacagcaagat
ctttttcgatccacctcctagagtaatggaaataa
aaacaaaaataaacaagtgggacctaatgaaactt
caaagcttttgcacagcaaaggaaaccataaacaa
gacgaaaagacaaccctcagaatgggagaaaatat
ttgcaaatgaatcaacggacaaaggattaatctcc
aaaatatataaacagctcattcagctcaatatcaa
agaaacaaacaccccaatccaaaaatgggcagaag
acctaaatagacatttctccaaagaagacatacag
acggccacgaagcacatgaaaagatgctcaacatc
actaattattagagaaatgcaaatcaaaactacaa
tgaggtatcacctcactcctgttagaatgggcatc
atcagaaaatctacaaacaacaaatgctggagagg
gtgtggagaaaagggaaccctcttgcactgttggt
gggaatgtaaattgatacagccactatggagaaca
atatggaggttccttaaaaaactaaaaatagaatt
accatatgacccagcaatcccactactgggcatat
acccagagaaaaccgtaattcaaaaagacacatgc
acccgaatgttcattgcagcactatttacaatagc
caggtcatggaagcaacctaaatgcccatcgacag
acgaatggataaagaagatgtggtacatatataca
atggaatattactcagccataaaaaggaacgaaat
tgggtcatttttagagacgtggatggatctagaga
ctgtcatacagagtgaagtaagtcagaaagagaaa
aacaaatatcgtatattaacgcatatatgtggaac
ctggaaaaatggtacagatgaaccggtctgcagga
cagaaattgagacacaaatgtaa.

In some embodiments, the construct comprises a nucleic acid sequence encoding a nuclear localization sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to PAAKRVKLD (SEQ ID NO: 63). In some embodiments, the nuclear localization sequence is fused to the ORF2p sequence. In some embodiments, the construct comprises a nucleic acid sequence encoding a flag tag having the sequence DYKDDDDK (SEQ ID NO: 64). In some embodiments, the flag tag is fused to the ORF2p sequence. In some embodiments, the flag tag is fused to the nuclear localization sequence.

In some embodiments, the construct comprises a nucleic acid sequence encoding an MS2 coat protein with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to ASNFTQFVLVDNGGTGDVTVAPSNFANGIAEWISSNSRSQAYKVTCSVRQSSAQNRKYTIKVEV PKGAWRSYLNMELTIPIFATNSDCELIVKAMQGLLKDGNPIPSAIAANSGIYAMASNFTQFVLVD NGGTGDVTVAPSNFANGIAEWISSNSRSQAYKVTCSVRQSSAQNRKYTIKVEVPKGAWRSYLN MELTIPIFATNSDCELIVKAMQGLLKDGNPIPSAIAANSGIY (SEQ ID NO: 65). In some embodiments, the MS2 coat protein sequence is fused to the ORF2p sequence.

In some embodiments, the transgene may comprise a flanking sequence which comprises an Alu ORF2p recognition sequence.

In some embodiments, additional elements may be introduced into the mRNA. In some embodiments, the additional elements may be an IRES element or a T2A element. In some embodiments, the mRNA transcript comprises one, two, three or more stop codons at the 3′-end.

In some embodiments, the one, two, three or more stop codons are designed to be in tandem. In some embodiments, the one, two, three or more stop codons are designed to be in all three reading frames.

In some embodiments, the one, two, three or more stop codons may be designed to be both in multiple reading frames and in tandem.

In some embodiments, one or more target specific nucleotides may be added at the priming end of the L1 or the Alu RNA priming region.

In some embodiments, the 5′ UTR sequence or the 3′ UTR sequence in addition to be able to bind the ORF protein may also be capable of binding to one or more endogenous proteins that regulate gene retrotransposition and/or stable integration. In some embodiments, the flanking sequence is capable of binding to a PABP protein.

In some embodiments, the 5′ region flanking the transcript may comprise a strong promoter. In some embodiments, the promoter is a CMV promoter.

In some embodiments, an additional nucleic encoding L1 ORF2p is introduced into the cell. In some embodiments, the sequence encoding L1 ORF1 is omitted, and only L1-ORF2 is included. In some embodiments, the nucleic acid encoding the transgene with the flanking elements is mRNA. In some embodiments, the endogenous L1-ORF1p function may be suppressed or inhibited.

In some embodiments, the nucleic acid encoding the transgene with the retrotransposition flanking elements comprise one or more nucleic acid modifications. In some embodiments, the nucleic acid encoding the transgene with the retrotransposition flanking elements comprises one or more nucleic acid modifications in the transgene. In some embodiments, the modifications comprise codon optimization of the transgene sequence. In some embodiments, the codon optimization is for more efficient recognition by the human translational machinery, leading to more efficient expression in a human cell. In some embodiments, the one or more nucleic acid modification is performed in the 5′-flanking sequence or the 3′—flanking sequence including one or more stem-loop regions. the nucleic acid encoding the transgene with the retrotransposition flanking elements comprise one, two, three, four, five, six, seven eight, nine, ten or more nucleic acid modifications.

In some embodiments, the retrotransposed transgene is stably expressed for the life of the cell. In some embodiments, the cell is a myeloid cell. In some embodiments, the myeloid cell is a monocyte precursor cell. In some embodiments, the myeloid cell is an immature monocyte. In some embodiments, the monocyte is an undifferentiated monocyte. In some embodiments, the myeloid cell is a CD14+ cell. In some embodiments, the myeloid cell does not express CD16 marker. In some embodiments, the myeloid cell is capable of remaining functionally active for a desired period of greater than 3 days, greater than 4 days, greater than 5 days, greater than 6 days, greater than 7 days, greater than 8 days, greater than 9 days, greater than 10 days, greater than 11 days, greater than 12 days, greater than 13 days, greater than 14 days or more under suitable conditions. A suitable condition may denote an in vitro condition, or an in vivo condition or a combination of both.

In some embodiments, the retrotransposed transgene may be stably expressed in the cell for about 2 days, about 3 days, about 4 days, about 5 days, about 6 days, about 7 days, about 8 days, about 9 days or about 10 days. In some embodiments, the retrotransposed transgene is stably expressed in the cell for more than 10 days. In some embodiments, the retrotransposed transgene is stably expressed in the cell for more than 2 weeks. In some embodiments, the retrotransposed transgene is stably expressed in the cell for about 1 month.

In some embodiments, the retrotransposed transgene may be modified for stable expression. In some embodiments, the retrotransposed transgene may be modified for resistant to in vivo silencing.

In some embodiments, the expression of the retrotransposed transgene may be controlled by a strong promoter. In some embodiments, the expression of the retrotransposed transgene may be controlled by a moderately strong promoter. In some embodiments, the expression of the retrotransposed transgene may be controlled by a strong promoter that can be regulated in an in vivo environment. In some embodiments, the promoter is a CMV promoter. In some embodiments, the promoter is a L1-Ta promoter.

In some embodiments, the ORF1p may be overexpressed. In some embodiments, the ORF2 may be overexpressed. In some embodiments, the ORF1p or ORF2p or both are overexpressed. In some embodiments, upon overexpression of an ORF1, ORF1p is at least 1.1 fold, 1.5 fold, 2 fold, 3 fold, 4 fold, 5 fold, 6 fold, 7 fold, 8 fold, 9 fold, 10 fold, 12 fold, 14 fold, 16 fold, 18 fold, 20 fold, 30 fold, 40 fold, 50 fold, 60 fold, 70 fold, 80 fold, 90 fold, or at least 100 fold higher than a cell not overexpressing and ORF1.

In some embodiments, upon overexpression of an ORF2 sequence, ORF2p is at least 1.1 fold, 1.5 fold, 2 fold, 3 fold, 4 fold, 5 fold, 6 fold, 7 fold, 8 fold, 9 fold, 10 fold, 12 fold, 14 fold, 16 fold, 18 fold, 20 fold, 30 fold, 40 fold, 50 fold, 60 fold, 70 fold, 80 fold, 90 fold, or at least 100 fold higher than a cell not overexpressing and ORF2p.

### Retrotransposition Fidelity and Target Specificity

The LINE-1 elements can bind to their own mRNA poly A tail to initiate retrotransposition. LINE-1 elements preferably retrotranspose their own mRNA over random mRNAs (Dewannieux et al., 2013, 3,000-fold higher LINE-1 retrotransposition as compared to random mRNAs). In addition, LINE-1 elements can also integrate non-specific poly-A sequences within a genome.

In one aspect, provided herein are retrotransposition compositions and methods of using the same with increased retrotransposition specificity. For example, retrotransposition compositions with high specificity may be used for highly specific and efficient reverse transcription and subsequently, integration into genome of a target cell, e.g., a myeloid cell. In some embodiments, a retrotransposition composition provided herein comprises a retrotransposition cassette that comprises one or more additional components that increases integration or retrotransposing specificity. For example, the retrotransposon cassette may encode one or more additional elements that allows for high affinity RNA-protein interaction to out compete non-specific binding between poly-A sequences and ORF2.

Accordingly, several measures are disclosed herein for enhancing integration or retrotransposition efficiency.

One exemplary measure for enhancing integration or retrotransposition efficiency is external manipulation of the cells. The endonuclease function of the retrotransposition machinery delivered in a cell may likely be subject to inhibition by the cell's transposition silencing machinery, such as DNA repair pathways. For example, small molecules can be used to modulate or inhibit DNA repair pathways in the cells prior to introducing the nucleic acid. For example, cell sorting and/or synchronization can be used prior to introducing the nucleic acid, such as by electroporation, as cell cycle synchronized cell populations were shown to increase gene transfer to the cells. Cell sorting may be utilized to synchronize or homogenize the cell types and increase uniform transfer and expression of the exogenous nucleic acid. Uniformity may be achieved sorting stem cells from non-stem cells. Another exemplary measure for enhancing integration or retrotransposition efficiency is to enhance biochemical activity. For example, this may be achieved by increasing reverse-transcriptase processivity or DNA cleavage (endonuclease) activity. Another exemplary measure for enhancing integration or retrotransposition efficiency is to subvert endogenous silencing mechanisms. For example, this may be achieved by replacing entire LINE-1 sequence with a different organisms' LINE-1. Another exemplary measure for enhancing integration or retrotransposition efficiency is to enhance translation and ribosome binding. For example, this may be achieved by increasing expression of LINE-1 proteins, increasing LINE protein binding LINE-1 mRNA, or increasing LINE-1 complex binding to ribosomes. Another exemplary measure for enhancing integration or retrotransposition efficiency is to increase nuclear import or retention. For example, this may be achieved by fusing the LINE-1 sequence to a nuclear retention signal sequence. Another exemplary measure for enhancing integration or retrotransposition efficiency is to enhance sequence-specific insertion. For example, this may be achieved by fusing a targeting domain to ORF2 to increase sequence specific retrotransposition.

In one embodiment, the method encompasses enhancing the retrotransposon for increasing specificity and robustness of expression of the cargo by modifying the UTR sequence of the LINE-1 ORFs.

In some embodiments, the 5′UTR upstream of ORF1 or ORF2 encoding sequence may be further modified to comprise a sequence that is complementary to the sequence of a target region within the genome that helps in homologous recombination at the specific site where the ORF nuclease can act and the retrotransposition can take place. In some embodiments, the sequence that can bind to a target sequence by homology is between 2-15 nucleotides long. In some embodiments, the sequence having homology to a genomic target that is included in the 5′UTR of an ORF1 mRNA may be about 3 nucleotides, about 4 nucleotides, about 5 nucleotides, about 6 nucleotides, about 7 nucleotides, about 8 nucleotides, about 9 nucleotides or about 10 nucleotides long. In some embodiments, the sequence having homology to a genomic target is about 12 or about 15 nucleotides long. In some embodiments, the sequence having homology to a genomic target is at least about 10, 15, 20, 25, 30, 35, 40, 45, 50, 55, 60, 65, 70, 75, 80, 85, 90, 95, 100, 105, 110, 115, 1120 or 125 nucleotides in length. In some embodiments, the sequence having homology to a genomic target comprises about 2-5, about 2-6, about 2-8 or about 2-10, or about 2-12 contiguous nucleotides that share complementarity with the respective target region within the genome. In some embodiments, the sequence having homology to a genomic target is at least about or about 10, 15, 20, 25, 30, 35, 40, 45, 50, 55, 60, 65, 70, 75, 80, 85, 90, 95, 100, 105, 110, 115, 1120 or 125 contiguous nucleotides that share complementarity with the respective target region within the genome.

In some embodiments, an ORF2 is associated with or fused to an additional protein domain that comprises RNA binding activity. In some embodiments, the retrotransposon cassette comprises a cognate RNA sequence that comprises affinity with the additional protein domain associated with or fused to the ORF2. In some embodiments, the ORF2 is associated with or fused to a MS2-MCP coat protein. In some embodiments, the retrotransposon cassette further comprises a MS2 hairpin RNA sequence in the 3′ or 5′ UTR sequence that interacts with the MS2-MCP coat protein. In some embodiments, the ORF2 is associated with or fused to a PP7 coat protein. In some embodiments, the retrotransposon cassette further comprises a PP7 hairpin RNA sequence in the 3′ or 5′ UTR sequence that interacts with the MS2-MCP coat protein. In some embodiments, the one or more additional elements increases retrotransposition specificity by at least 1.5 fold, at least 2 fold, at least 3 fold, at least 4 fold, at least 5 fold, at least 10 fold, at least 20 fold, at least 30 fold, at least 50 fold, at least 100 fold, at least 200 fold, at least 300 fold, at least 500 fold, at least 1000 fold, at least 1500 fold, at least 2000 fold, at least 3000 fold, at least 5000 fold or more as compared to a retrotransposon cassette without the one or more additional elements.

The DNA endonuclease domain appears to have specificity for a series of purines 3′ of the target site followed by a series of pyrimidines (Py)4 (Pu).. An exemplary sequence may be (Adenosine)4l(Thymidine)ₙ.

In one aspect, provided herein are methods of using retrotransposition having high target specificity. In some embodiments, a CRISPR-Cas guide RNA system is combined with the LINE-retrotransposon system used here to increase the precision of site directed retrotransposition. In some embodiments, the system incorporates a prime editing guide RNA (pegRNA) to incorporate one or more ORF-binding sequence into a specific genomic locus. In some embodiments, the pegRNA incorporates the sequence that binds human ORF, e.g. TTTTTA in a site-specific manner. In some embodiments, the CRISPR-Cas system comprises a Cas9 enzyme. In some embodiments the CRISPR-Cas comprises a Cfpl enzyme. In some embodiments, the Cas9 is a dCas9, paired with a nickase system.

Consequently, provided herein is a method and compositions for stable incorporation of a transgene into the genome of a myeloid cell, such as a monocyte or macrophage, wherein the method comprises incorporating the transgene using a non-LTR retrotransposon system, wherein the retrotransposition occurs at a specific genomic locus with a target specificity, high precision and fidelity.

Therefore, in some embodiments, the method comprises administration to the cell a composition comprising a system having at least one transgene, flanked with one or more retrotransposable elements, and one or more nucleic acids encoding one or more proteins for increasing the transposition specificity, and/or further comprising modifying one or more genes associated with the retrotransposition.

The nucleic acid comprising the transgene, situated in 3′ UTR region of the retrotransposable elements is often referred to as a retrotransposition cassette. Accordingly, in some embodiments, the retrotransposition cassette comprises the nucleic acid encoding the transgene and flanking Alu transposable elements. The retrotransposable elements comprise a sequence for binding the retrotransposons, for example, L1-transposons, such as L1-ORF proteins, ORF1p and ORF2p. ORF proteins are known to bind to their own mRNA sequence for retrotransposition. Therefore, the retrotransposition cassette comprises the nucleic acid encoding the transgene; a flanking L1-ORF2p binding sequence, and/or a L1-ORF1p binding sequence, comprising a sequence encoding a L1-ORF1p encoding sequence and a L1-ORF2p encoding sequence outside the transgene sequence. In some embodiments, the L1-ORF1 and L1-ORF2 are interspersed by a spacer region, also termed as an ORF1—ORF2 inter-region. In some embodiments, the L1-ORF1 and L1-ORF2 coding sequences are in an opposite orientation with respect to the coding region of the transgene. The retrotransposition cassette can comprise a poly A region downstream of the L1-ORF2-coding sequence and the transgene sequence is placed downstream of the poly A sequence. The L1-ORF2 comprises a nucleic acid sequence that encodes an endonuclease (EN) and a reverse transcriptase (RT) followed by the poly A sequence. In some embodiments, the L1-ORF2 sequence in the retrotransposition cassette described herein is a complete (intact) sequence, that is, encodes the full length native (WT) L1-ORF2 sequence. In some embodiments, the L1-ORF2 sequence in the retrotransposition cassette described herein comprises a partial or modified sequence.

The system described herein can comprise a promoter for expressing the L1-ORF1p and L1-ORF2p. In some embodiments, the transgene expression is driven by a separate promoter. In some embodiments, the transgene and the ORFs are in tandem orientation. In some embodiments, the transgene and the ORFs are in opposite orientation.

In some embodiments, the method comprises incorporating one or more elements in addition to the retrotransposon cassette. In some embodiments, the one or more additional elements comprise a nucleic acid sequence encoding one or more domains of a heterologous protein. The heterologous protein may be a sequence specific nucleic acid binding protein, for example, a sequence specific DNA binding protein domain (DBD). In some embodiments, the heterologous protein is a nuclease or a fragment thereof. In some embodiments, the additional elements comprise a nucleic acid sequence encoding one or more nuclease domains or fragments thereof from a heterologous protein. In some embodiments, the heterologous nuclease domain has reduced nuclease activity. In some embodiments, the heterologous nuclease domain is rendered inactive. In some embodiments, the ORF2 nuclease is rendered inactive; whereas one or more nuclease domains from the heterologous protein is configured to render specificity to the retrotransposition. In some embodiments, one or more nuclease domains or fragments thereof from the heterologous protein targets a specific desired polynucleotide within the genome where retrotransposition and incorporation of the polynucleotide of interest is to be incorporated. In some embodiments, the one or more nuclease domains from the heterologous protein comprise a mega-TAL nuclease domain, TALENs, or a zinc finger nuclease domain, for example, a mega-TAL, a TALE, or a zinc finger domain fused to or associated with a nuclease domain, e.g., a FokI nuclease domain. In some embodiments, the one or more nuclease domains from the heterologous protein comprise a CRISPR-Cas protein domain loaded with a specific guide nucleic acid, e.g., a guide RNA (gRNA) for a specific target locus. In some embodiments, the CRISPR-Cas protein is a Cas9, a Cas12a, a Cas12b, a Cas13, a CasX, or a CasY protein domain. In some embodiments, the one or more nuclease domains from the heterologous protein has target specificity.

In some embodiments, the additional nuclease domain may be incorporated into the ORF2 domain. In some embodiments, the additional nuclease may be fused with the ORF2p domain. In some embodiments, the additional nuclease domain may be fused to an ORF2p, wherein the ORF2p includes a mutation in the ORF2p endonuclease domain. In some embodiments, the mutation inactivates the ORF2p endonuclease domain. In some embodiments, the mutation is a point mutation. In some embodiments, the mutation is a deletion. In some embodiments, the mutation is an insertion. In some embodiments, the mutation abrogates the ORF2 endonuclease (nickase) activity. In some embodiments, a mutation inactivates the DNA target recognition of ORF2p endonuclease. In some embodiments, the mutation covers a region associated with ORF2p nuclease-DNA recognition. In some embodiments, a mutation reduces the DNA target recognition of ORF2p endonuclease. In some embodiments, the ORF2p endonuclease domain mutation is in the N-terminal region of the protein. In some embodiments, the ORF2p endonuclease domain mutation is in a conserved region of the protein. In some embodiments, the ORF2p endonuclease domain mutation is in the conserved N-terminal region of the protein. In some embodiments, the mutation comprises the N14 amino acid within L1 endonuclease domain. In some embodiments, the mutation comprises 2, 3, 4, 5, 6, 7, 8, 9, 10, 11, 12, 13, 14, 15, 16, 17, 18, 19, 20 or more consecutive amino acids including the N14 amino acid within L1 endonuclease domain. In some embodiments, the mutation comprises the comprises the E43 amino acid within L1 endonuclease. In some embodiments, the mutation comprises 2, 3, 4, 5, 6, 7, 8, 9, 10, 11, 12, 13, 14, 15, 16, 17, 18, 19, 20 or more consecutive amino acids including the E43 amino acid within Li endonuclease domain. In some embodiments, the mutation comprises 2 or more amino acids in the L1 endonuclease domain including N14, or E43 or a combination thereof. In some embodiments, the mutation comprises D145 of the L1 endonuclease domain. In some embodiments, the mutation may be D145A. In some embodiments, the may be a comprise D205 of the L1 endonuclease domain. In some embodiments, the mutation may be D205G. In some embodiments, the mutation may comprise H230 of L1 endonuclease domain. In some embodiments, the may be a comprise S228 of the L1 endonuclease domain. In some embodiments, the mutation may be S228P.

In some embodiments, a mutation reduces the DNA target recognition of ORF2p endonuclease by at least 50%. In some embodiments, a mutation reduces the DNA target recognition of ORF2p endonuclease by at least 60%. In some embodiments, a mutation reduces the DNA target recognition of ORF2p endonuclease by at least 70%. In some embodiments, a mutation reduces the DNA target recognition of ORF2p endonuclease 80%. In some embodiments, a mutation reduces the DNA target recognition of ORF2p endonuclease 90%. In some embodiments, a mutation reduces the DNA target recognition of ORF2p by 95%. In some embodiments, a mutation reduces the DNA target recognition of ORF2p by 100%.

In some embodiments, the mutation is a deletion. In some embodiments, the deletion is complete, i.e., 100% of the L1 endonuclease domain is deleted. In some embodiments, the deletion is partial. In some embodiments, the about 98%, about 95%, about 94%, about 93%, about 92% about 91%, about 90%, about 85%, about 80%, about 75%, about 70%, about 65%, about 60%, or about 50% of the ORF2 endonuclease domain is deleted.

In some embodiments, an additional nuclease domain is inserted into the ORF2 protein sequence. In some embodiments, ORF2 endonuclease domain is deleted, and is replaced with an endonuclease domain from a heterologous protein. In some embodiments, the ORF2 endonuclease is partially deleted and replaced with an endonuclease domain from a heterologous protein. The endonuclease domain from a heterologous protein may be a mega-TAL nuclease domain. The endonuclease domain from a heterologous protein may be a TALENs. The endonuclease domain from a heterologous protein may be a Cas9 loaded with a specific gRNA for a locus.

In some embodiments, the endonuclease is an endonuclease that has (i) a specific target on the genome and (ii) it creates a 5′-P and a 3′-OH terminus at the cleavage site.

In some embodiments, the additional endonuclease domain from a heterologous protein is an endonuclease domain from a related retrotransposon.

In some embodiments, the endonuclease domain from a heterologous protein may comprise a bacterial endonuclease engineered for targeting a specific site. In some embodiments, the endonuclease domain from a heterologous protein may comprise a domain of a homing endonuclease or a fragment thereof. In some embodiments, the endonuclease is a homing endonuclease. In some embodiments, the homing endonuclease is an engineered LAGLIDADG homing endonucleases (LHEs) (“LAGLIDADG” disclosed as SEQ ID NO: 66) or a fragment thereof. In some embodiments, additional endonucleases may be a restriction endonuclease, Cre, Cas TAL or fragments thereof. In some embodiments, the endonuclease may comprise a Group II intron encoded protein (ribozyme) or a fragment thereof.

An engineered or modified L1-ORF2p as discussed in the preceding paragraphs, that is endowed with specific DNA targeting capability due to the additional/heterologous endonuclease is expected to be highly advantageous in driving targeted stable integration of a transgene into the genome. The engineered L1-ORF2p can generate much reduced off-target effects when expressed in a cell than using a native, non-engineered L1-ORF2p. In some embodiments, the engineered L1-ORF2p generates no off-target effect.

In some embodiments, the engineered or modified L1-ORF2p targets a recognition site that is other than the usual (Py)4 (Pu)˜site. In some embodiments, engineered L1-ORF2p targets a recognition site that comprises the (Py)4j(Pu) site, for example, TTTT/AA site, such as a hybrid target site. In some embodiments, the engineered L1-ORF2p targets a recognition site having at least one nucleotide in addition to the conventional L1-ORF2 (Py).J(Pu)˜site, for example TTTT/AAG, or TTTT/AAC, or TTTT/AAT, TTTT/AAA, GTTTT/AA, CTTTT/AA, ATTTT/AA, or TTTTT/AA. In some embodiments, the engineered L1-ORF2p targets a recognition site that is in addition to the conventional L1-ORF2p (Py)₁4(Pu), site. In some embodiments, the engineered L1-ORF2p targets a recognition site that is other than to the conventional L1-ORF2p (Py).J(Pu), site. In some embodiments, the engineered L1-ORF2p targets a recognition site that is 4, 5, 6, 7, 8, 9, 10 or more nucleotides long. In some embodiments, the engineered or modified L1-ORF2p recognition site may be 11, 12, 13, 14, 15, 16, 17, 18, 19, 20 or more nucleotides.

The engineered L1-ORF2p can be engineered to retain its ability to bind to its own mRNA after translation and reverse transcribe with high efficiency. In some embodiments, the engineered L1-ORF2p has enhanced efficiency of reverse transcription compared to a native (WT) L1-ORF2p.

In some embodiments, the system comprising a retrotransposable element further comprises a gene modification that reduces non-specific retrotransposition. In some embodiments, the gene modification may comprise a sequence encoding the L1-ORF2p. In some embodiments, the modification may comprise mutation of one or more amino acids that are essential for binding to a protein that helps ORF2p binding to the target genomic DNA. A protein that helps ORF2p binding to the target genomic DNA may be part of the chromatin-ORF interactome. In some embodiments, the modification may comprise one or more amino acids that are essential for binding to a protein that helps ORF2p DNA endonuclease activity. In some embodiments, the modification may comprise one or more amino acids that are essential for binding to a protein that helps ORF2p RT activity. In some embodiments, the modification may comprise at a protein binding site on ORF2p such that the association of a protein with ORF2p is altered, wherein binding of the protein to ORF2p is required for binding to chromatin. In some embodiments, the modification may comprise at a protein binding site on ORF2p such that the association of the protein with ORF2p is more stringent and/or specific than in absence of the modification. In some embodiments, as a consequence of altered association of ORF2p with the protein owing to the modification of ORF2p coding sequence at the protein binding site, the binding of ORF2p to the target DNA has increased specificity. In some embodiments, the modification may reduce binding of ORF2 to one or more proteins that are part of the ORF2p chromatin interactome.

In some embodiments, the gene modification may be in the PIP domain of ORF2p.

In some embodiments, the gene modification may be in one or more genes encoding a protein that binds to an ORF2p and helps in the recognition, binding, endonuclease or RT activity of ORF2p. In some embodiments, the gene modification may be in one or more genes encoding PCNA, PARP1, PABP, MCM, TOP1, RPA, PURA, PURB, RUVBL2, NAP1, ZCCHC3, UPF1 or MOV10 proteins at an ORF2p interacting site for each protein or at a site that affects the protein's interaction with ORF2p or the interaction of ORF2p with target DNA. In some embodiments, the modification may be on an ORF2p binding domain of PCNA at an ORF2p interacting site or at a site that affects the protein's interaction with ORF2p or the interaction of ORF2p with target DNA. In some embodiments, the modification may be on an ORF2p binding domain of TOP1. In some embodiments, the modification may be on an ORF2p binding domain of RPA. In some embodiments, the modification may be on an ORF2p binding domain of PARP1 at an ORF2p interacting site or at a site that affects the protein's interaction with ORF2p or the interaction of ORF2p with target DNA. In some embodiments, the modification may be on an ORF2p binding domain of PABP (e.g., PABPC1) at an ORF2p interacting site or at a site that affects the protein's interaction with ORF2p or the interaction of ORF2p with target DNA. In some embodiments, the gene modification may be on an MCM gene. In some embodiments, the gene modification may be on a gene encoding MCM3 protein at an ORF2p interacting site or at a site that affects the protein's interaction with ORF2p or the interaction of ORF2p with target DNA. In some embodiments, the gene modification may be on a gene encoding MCM5 protein at an ORF2p interacting site or at a site that affects the protein's interaction with ORF2p or the interaction of ORF2p with target DNA. In some embodiments, the gene modification may be on a gene encoding MCM6 protein at an ORF2p interacting site or at a site that affects the protein's interaction with ORF2p or the interaction of ORF2p with target DNA. In some embodiments, the gene modification may be on a gene encoding MEPCE protein at an ORF2p interacting site or at a site that affects the protein's interaction with ORF2p or the interaction of ORF2p with target DNA. In some embodiments, the gene modification may be on a gene encoding on a gene encoding RUVBL1 or RUVBL2 protein at an ORF2p interacting site or at a site that affects the protein's interaction with ORF2p or the interaction of ORF2p with target DNA. In some embodiments, the gene modification may be on a gene encoding on a gene encoding TROVE protein at an ORF2p interacting site or at a site that affects the protein's interaction with ORF2p or the interaction of ORF2p with target DNA.

In some embodiments, the retrotransposition system disclosed herein comprises one or more elements that increase the fidelity of reverse transcription.

In some embodiments, the L1-ORF2 RT domain is modified. In some embodiments, the modification includes one or more of: increasing fidelity, increasing processivity, increasing DNA-RNA substrate affinity; or inactivating RNase H activity.

In some embodiments, the modification comprises introducing one or more mutations in the RT domain of the L1-ORF2, such that the fidelity of the RT is increased. In some embodiments, the mutation comprises a point mutation. In some embodiments, the mutation comprises alteration, such as substitution of one, two three, four, five, six or more amino acids in the L1-ORF2p RT domain. In some embodiments, the mutation comprises deletion of one or more amino acids, for example, one, two, three, four, five, six, seven, eight, nine, ten or more amino acids in the L1-ORF2p RT domain. In some embodiments, the mutation may comprise an in-del mutation. In some embodiments, the mutation may comprise a frame-shift mutation.

In some embodiments, the modification may comprise inclusion of an additional RT domain or fragment thereof from a second protein. In some embodiments, the second protein is a viral reverse transcriptase. In some embodiments, the second protein is a non-viral reverse transcriptase. In some embodiments, the second protein is a retrotransposable element. In some embodiments, the second protein is a non-LTR retrotransposable element. In some embodiments, the second protein is a group II intron protein. In some embodiments, the group II intron is as TGIRTII. In some embodiments, the second protein is a Cas nickase, wherein the retrotransposable system further comprises introducing a guide RNA. In some embodiments, the second protein is a Cas9 endonuclease, wherein the retrotransposable system further comprises introducing a guide RNA. In some embodiments, the second protein or fragment thereof is fused to the N-terminus of the L1-ORF2 RT domain or the modified L1-ORF2 RT domain. In some embodiments, the second protein or fragment thereof is fused to the C-terminus of the L 1-ORF2 RT domain or the modified L1-ORF2 RT domain.

In some embodiments, the additional RT domain or fragment thereof from the second protein is incorporated in the retrotransposition system in addition to the full-length WT L1-ORF2p RT domain. In some embodiments, the additional RT domain or fragment thereof from the second protein is incorporated in presence of a modified (engineered) L1-ORF2p RT domain or a fragment thereof, where the modification (or engineering) may comprise a mutation for enhancement of the L1-ORF2p RT processivity, stability and/or fidelity of the modified L1-ORF2p RT compared to the native or WT ORF2p.

In some embodiments, the reverse transcriptase domain could be replaced with other more highly processive and high-fidelity RT domains from other retroelements or group II introns, such as TGIRTII.

In some embodiments, the modification may comprise a fusion with an additional RT domain or fragment thereof from a second protein. In some embodiments, the second protein may comprise a retroelement. The additional RT domain or fragment thereof from a second protein is configured to increase the fidelity of reverse transcription of the fused L1-ORF2p RT domain. In some embodiments, the nucleic acid encoding the additional RT domain or fragment thereof is fused to a native or WT L1-ORF2 encoding sequence. In some embodiments, the nucleic acid encoding the additional RT domain or fragment thereof from a second protein is fused to a modified L1-ORF2 encoding sequence. In some embodiments, the modification comprises introducing one or more mutations in the RT domain of the L1-ORF2 or fragment thereof, such that the fidelity of the fused RT is increased. In some embodiments, the mutation in the RT domain of the L1-ORF2 or fragment thereof comprises a point mutation. In some embodiments, the mutation comprises alteration, such as substitution of one, two three, four, five, six or more amino acids in the L1-ORF2p RT domain. In some embodiments, the mutation comprises deletion of one or more amino acids, for example, one, two, three, four, five, six, seven, eight, nine, ten or more amino acids in the L1-ORF2p RT domain. In some embodiments, the mutation may comprise an in-del mutation. In some embodiments, the mutation may comprise a frame-shift mutation.

In some embodiments, the modified L1-ORF2p RT domain has increased processivity than the WT L1—ORF2p RT domain.

In some embodiments, the modified L1-ORF2p RT domain has at least 10% higher processivity and/or fidelity over the WT L1—ORF2p RT domain. In some embodiments, the modified L1-ORF2p RT domain has at least 20%, 30%, 40%, 50%, 60%, 70%, 80%, 90%, 100%, 110%, 120%, 150%, 200%, 300%, 400%, 500%, 1000% or higher processivity and/or fidelity over the WT L1—ORF2p RT domain. In some embodiments, the modified RT can process greater than 6kb nucleic acid stretch. In some embodiments, the modified RT can process greater than 7kb nucleic acid stretch. In some embodiments, the modified RT can process greater than 8kb nucleic acid stretch. In some embodiments, the modified RT can process greater than 9kb nucleic acid stretch. In some embodiments, the modified RT can process greater than 10kb nucleic acid stretch.

### B. Group II Introns and Ribozvmes

Group II enzymes are mobile ribozymes that self-splice precursor RNAs, yielding excised intron lariat RNAs. The introns encode a reverse transcriptase. The reverse transcriptase may stabilize the RNA for forward and reverse splicing, and later in converting the integrated intron RNA to DNA.

Group II RNAs are characterized by a conserved secondary structure spanning 400-800 nucleotides. The secondary structure is formed by six domains DI-VI, and is organized in a structure resembling a wheel, where the domains radiate from a central point. The domains interact to form a conserved tertiary structure that brings together distant sequences to form an active site. The active site binds the splice sites and branch point residue nucleotide and in association of Mg2+ cations, activate catalysis of splicing. The DV domain is within the active site, which has the conserved catalytic AGC and an AY bulge and both these regions bind Mg2+ ions necessary for the catalysis. DI is the largest domain with upper and lower halves separated by kappa and zeta motifs. The lower half contains the ε′ motif, which is associated with an active site. The upper half contains sequence elements that bind to the 5′ and 3′ exons at the active sites. DIV encodes the intron-encoded protein (IEP) with subdomain IVa near the 5′-end containing the high affinity binding site for IEP. Group II introns have conserved 5′- and 3′—end sequences, GUGYG and AY respectively.

Group II RNA introns can be utilized to retrotranspose a sequence of interest into DNA via target primed reverse transcription. This process of transposition by Group II RNA introns is often referred to as retrohoming. Group II introns recognize DNA target sites by base pairing of the intron RNA to the DNA target sequence, they can be modified to retarget a specific sequence carried within the intron to a desired DNA site.

In some embodiments, the method and compositions for retrotransposition described herein may comprise a Group II intron sequence, a modified Group II intron sequence or a fragment thereof.

Exemplary Group II IEPs (maturase) include but are not limited to bacterial, fungal, yeast IEPs, that are functional in human cells. In particular, the nuclease leaves a 3′-OH at the cleavage site of the DNA which can be utilized by another RT for priming and reverse transcription. An exemplary Group II maturase may be TGIRT (thermally stable group II intron maturase).

In one or more embodiments of several aspects described herein, the nucleic acid construct comprises an RNA. In one or more embodiments of several aspects of the disclosure, the nucleic acid construct is an RNA. In one or more embodiments of several aspects of the disclosure, the nucleic acid construct is an mRNA. In one aspect, the mRNA comprises a sequence of a heterologous gene or portion thereof, wherein the heterologous gene or portion thereof encodes a polypeptide or protein. In some embodiments, the mRNA comprises a sequence encoding a fusion protein. In some embodiments, the mRNA comprises a sequence encoding a recombinant protein. In some embodiments, the mRNA comprises a sequence encoding a synthetic protein. In some embodiments, the nucleic acid comprises one or more sequences, wherein the one or more sequences encode on or more heterologous proteins, one or more recombinant proteins, or one or more synthetic proteins or a combination thereof. In some embodiments, the nucleic acid comprises one or more sequences, wherein the one or more sequences encode on or more heterologous proteins comprising a synthetic protein or a recombinant protein. In some embodiments, the synthetic or recombinant protein is a recombinant fusion protein.

C. Retrotransposon systems comprising an site directed editing and/or integrase

In one aspect, provided herein are methods of using retrotransposition having high target specificity following modification over the pegRNA mediated incorporation of LINE binding sequences site-specifically into the genome, with the help of guide RNA and a Cas protein. In some embodiments, a CRISPR-Cas guide RNA system is combined with the LINE-retrotransposon system used here to increase the precision of site directed retrotransposition; for example, the system incorporates a prime editing guide RNA (pegRNA) to incorporate one or more ORF-binding sequence into a specific genomic locus. In some embodiments, the pegRNA incorporates the sequence that binds human ORF, e.g. TTTTTA in a site-specific manner. In some embodiments, the CRISPR-Cas system comprises a Cas9 enzyme. In some embodiments the CRISPR-Cas comprises a Cfpl enzyme. In some embodiments, the Cas9 is a dCas9, paired with a nickase system.

In some embodiments, the retrotransposon systems described herein comprise (i) a LINE1 retrotransposon element, and (ii) an integrase system or parts thereof. Some integrase systems are capable of site-specific integration—of double stranded DNA. In order to bypass the double stranded DNA delivery and/or integration into a genome, provided herein is a recombinant hybrid system wherein the integrase or a fragment thereof incorporated within a recombinant ORF protein, or delivered separately as a separate nucleic acid (e.g. mRNA) encoding an integrase or fragment thereof that recognizes a specific genomic site; and couples with the LINE 1 reverse transcription and insertion of a cargo sequence within the genome of a cell or an organism at the precise location led by the specificity of the integrase. This could be achieved in a few alternative ways. In some embodiments, the cargo sequence comprises an attachment site that is recognized and utilized by the integrase to draw the cargo to the landing site within the genome, also recognized by the same integrase. The integrase is capable of a single strand cut. The integrase DNA recognition site, i.e., the genomic landing sequence, can be 10 nucleotides long, e.g., 12, 14, 16, 18, 20, 22, 24, 26, 28, 30 or more nucleotides long, thereby conferring greater specificity than any other system. The integrase may be truncated or otherwise mutated to allow the ORF to reverse transcribe and integrate the cargo sequence at the integrase specified genomic site. Conversely, the ORF protein may also be mutated at the RNA recognition site to allow the integrase recognize the genomic integration sequence preferentially recognized by the integrase (also termed the “genomic landing sequence or site”. In alternative embodiments, the integrase is encoded by a separate polynucleotide, and may be driven by a CRISPR Cas system and a guide RNA to a site that can be nicked and an integrase landing sequence further comprising an ORF binding site comprising the 4 nucleotides may be introduced, thereafter the integrase draws in the cargo sequence that comprises the attachment sequence to the landing sequence, followed by the LINE1 activity leading to genomic integration at the site specified by the integrase system. Any catalytic activity of the integrase leading to double-stranded DNA incorporation in the genome is mutated or truncated, otherwise, silenced.

In one or more of embodiments of several aspects of the disclosure, the nucleic acid construct is developed for expressing in a eukaryotic cell. In some embodiments, the nucleic acid construct is developed for expressing in a human cell. In some embodiments, the nucleic acid construct is developed for expressing in a hematopoietic cell. In some embodiments, the nucleic acid construct is developed for expressing in a myeloid cell. In some embodiments, the myeloid cell is a human cell.

II. Modifications in nucleic acid constructs for methods of enhancement of expression of encoded protein

In some aspects of the disclosure, the recombinant nucleic acid is modified for enhanced expression of the protein encoded by a sequence of the nucleic acid. Enhanced expression of the protein encoded therein can be a function of the nucleic acid stability, translation efficiency and the stability of the translated protein. A number of modifications are contemplated herein for incorporation in the design of the nucleic acid construct that can confer nucleic acid stability, such as stability of the messenger RNA encoding the exogenous or heterologous protein, which may be a synthetic recombinant protein or a fragment thereof.

In some embodiments, the nucleic acid is mRNA, comprising one or more sequences, wherein the one or more sequences encode one or more heterologous proteins comprising a synthetic or a recombinant fusion protein.

In some embodiments, one or more modifications are made in the mRNA comprising a sequence encoding a recombinant or fusion protein to increase the mRNA half-life.

### Structural Elements to Block 5′- and 3′—Degradations by Exonucleases: 5′-Cap and 3′ UTR Modifications

A proper 5′—cap structure is important in the synthesis of functional messenger RNA. In some embodiments, the 5′—cap comprises a guanosine triphosphate arranged as GpppG at the 5′terminus of the nucleic acid. In some embodiments, the mRNA comprises a 5′ 7-methylguanosine cap, m7-GpppG. A 5′ 7-methylguanosine cap increases mRNA translational efficiency and prevents degradation of mRNA 5′-3′exonucleases. In some embodiments, the mRNA comprises “anti-reverse” cap analog (ARCA, m,^{7,3}′-° GpppG). Translational efficiency, however, can be markedly increased by usage of the ARCA. In some embodiments, the guanosine cap is a Cap 0 structure. In some embodiments, the guanosine cap is a Cap 1 structure. In addition to its essential role of cap-dependent initiation of protein synthesis, the mRNA cap also functions as a protective group from 5′ to 3′ exonuclease cleavage and a unique identifier for recruiting protein factors for pre-mRNA splicing, polyadenylation and nuclear export. It acts as the anchor for the recruitment of initiation factors that initiate protein synthesis and the 5′ to 3′ looping of mRNA during translation. Three enzymatic activities are required to generate the Cap 0 structure, namely, RNA triphosphatase (TPase), RNA guanylyltransferase (GTase) and guanine-N7 methyltransferase (guanine-N7 MTase). Each of these enzyme activities carries out an essential step in the conversion of the 5′ triphosphate of nascent RNA to the Cap 0 structure. RNA TPase removes the y-phosphate from the 5′ triphosphate to generate 5′ diphosphate RNA. GTase transfers a GMP group from GTP to the 5′ diphosphate via a lysine-GMP covalent intermediate. The guanine-N7 MTase then adds a methyl group to the N7 amine of the guanine cap to form the cap 0 structure. For Cap 1 structure, m7G-specific 2′O methyltransferase (2′O MTase)methylates the +1 ribonucleotide at the 2′O position of the ribose to generate the cap 1 structure.

The nuclear RNA capping enzyme interacts with the polymerase subunit of RNA polymerase II complex at phosphorylated Ser5 of the C-terminal heptad repeats. RNA guanine-N7 methyltransferase also interacts with the RNA polymerase II phosphorylated heptad repeats. In some embodiments, the cap is a G-quadruplex cap.

In some embodiments, the mRNA is synthesized by in vitro transcription (IVT). In some embodiments, mRNA synthesis and capping may be performed in one step. Capping may occur in the same reaction mixture as IVT. In some embodiments, mRNA synthesis and capping may be performed in separate steps. mRNA thus formed by IVT is purified and then capped.

In some embodiments, the nucleic acid construct, e.g., the mRNA construct, comprises one or more sequences encoding a protein or a polypeptide of interest can be designed to comprise elements that protect, prevent, inhibit or reduce degradation of the mRNA by endogenous 5′-3′ exoribonucleases, for example, Xrn1. Xrn1 is a cellular enzyme in the normal RNA decay pathways that degrades 5′ monophosphorylated RNAs. However, some viral RNA structural elements are found to be particularly resistant to such RNases, for example, the Xrn1-resistant structure in flavivirus sfRNAs, called the ‘xrRNA’. For example, the mosquito-borne flaviviruses (MBFV) genomes contain discrete RNA structures in their 3′-untranslated region (UTR) that block the progression of Xrn1. These RNA elements are sufficient to block Xrn1 without the use of accessory proteins. xrRNAs halt the enzyme at a defined location such that the viral RNA located downstream of the xrRNAs is protected from degradation. The xrRNAs from Zika virus or Murray Valley encephalitis virus, for example, comprise three-way junction and multiple pseudoknot interactions that create an unusual and complex fold that requires a set of nucleotides conserved across the MBFVs structure. xrRNAs halt the enzyme at a defined location such that the viral RNA located downstream of the xrRNAs is protected from degradation. The 5′-end of the RNA passes through a ring-like structure of the fold and is believed to remain protected from the Xrn1-like exonuclease.

In some embodiments, the nucleic acid construct comprising the one or more sequences that encode a protein of interest may comprise one or more xrRNA structures incorporated therein. In some embodiments, the xrRNA is a stretch of nucleotides having the conserved regions of the 3′ UTR of one or more viral xrRNA sequences. In some embodiments, 1, 2, 3, 4, 5, 6, 7, 8, 9, 10 or more xrRNA elements are incorporated within the nucleic acid construct. In some embodiments, 2 or more xrRNA elements are incorporated in tandem within the nucleic acid construct. In some embodiments, the xrRNA comprise one or more regions comprising conserved sequences or fragments thereof or modifications thereof. In some embodiments, the xrRNA is placed at the 3′UTR of a retrotransposon element. In some embodiments, the xrRNA is placed at upstream of the sequences encoding the one or more proteins or polypeptides. In some embodiments, the xrRNA is placed in the 3′UTR of a retrotransposon element, such as an ORF2 sequence, and upstream of the sequences encoding the one or more proteins or polypeptides.

In some embodiments, the xrRNA structure comprises a MBFV xrRNA sequence, or a sequence that is at least 90% identical thereof. In some embodiments, the xrRNA structure comprises a tick-borne flaviviruses (TBFVs) xrRNA sequence, or a sequence that is at least 90% identical thereof. In some embodiments, the xrRNA structure comprises a tick-borne flaviviruses (TBFVs) xrRNA sequence, or a sequence that is at least 90% identical thereof. In some embodiments, the xrRNA structure comprises a tick-borne flaviviruses (TBFVs) xrRNA sequence, or a sequence that is at least 90% identical thereof. In some embodiments, the xrRNA structure comprises a xrRNA sequence from a member of no known arthropod vector flaviviruses (NKVFVs), or a sequence that is at least 90% identical thereof. In some embodiments, the xrRNA structure comprises a xrRNA sequence from a member of insect-specific flaviviruses (ISFVs), or a sequence that is at least 90% identical thereof. In some embodiments, the xrRNA structure comprises a Zikavirus xrRNA sequence, or a sequence that is at least 90% identical thereof. It is hereby contemplated that any known xrRNA structural elements or conceivable non-obvious variations thereof may be used for the purpose described herein.

Several messenger RNAs from different organisms exhibit one or more pseudoknot structures that exhibits resistance from 5′-3′ exonuclease. A pseudoknot is a RNA structure that is minimally composed of two helical segments connected by single-stranded regions or loops. Although several distinct folding topologies of pseudoknots exist.

### Poly a Tail Modifications

The poly A structure in the 3′UTR of an mRNA is an important regulator of mRNA half-life. Deadenylation of the 3′ end of the poly A tail is the first step of the intracellular mRNA degradation. In some embodiments, the length of the poly A tail of the mRNA construct is taken into critical consideration and designed for maximizing the expression of the protein encoded by the mRNA coding region, and the mRNA stability. In some embodiments, the nucleic acid construct comprises one or more poly A sequences. In some embodiments, the poly A sequence at the 3′UTR of the sequences encoding the one or more proteins or polypeptides comprise 20-200 adenosine nucleobases. In some embodiments, the poly A sequence comprises 30-200 adenosine nucleobases. In some embodiments, the poly A sequence comprises 50-200 adenosine nucleobases. In some embodiments, the poly A sequence comprises 80-200 adenosine nucleobases. In some embodiments, the mRNA segment comprising the sequences that encode one or more proteins or polypeptides comprises a 3′-UTR having a poly-A tail comprising about 180 adenosine nucleobases, or about 140 adenosine nucleobases, or about 120 adenosine nucleobases. In some embodiments, the poly A tail comprises about 122 adenosine nucleobases. In some embodiments, the poly A sequence comprises 50 adenosine nucleobases. In some embodiments, the poly A sequence comprises 30 adenosine nucleobases. In some embodiments, the adenosine nucleobases in the poly A tail are placed in tandem, with or without intervening non-adenosine bases. In some embodiments, one or more non-adenosine nucleobases are incorporated in the poly A tail, which confer further resistance to certain exonucleases.

In some embodiments, the stretch of adenosines in poly A tail of the construct comprises one or more non-adenosine (A) nucleobase. In some embodiments, the non-A nucleobase is present at −3, −2, −1, and/or +1 position at the poly A 3′-terminal region. In some embodiments, the non-A bases comprise a guanosine (G) or a cytosine (C) or an uracil base (U). In some embodiments, the non-A base is a G. In some embodiments, the non-A base more than one, in tandem, for example, GG. In some embodiments, the modification at the 3′ end of the poly A tail with one or more non-A base is directed at disrupting the A base stacking at the poly A tail. The poly A base stacking promotes deadenylation by various deadenylating enzymes, and therefore 3′ end of poly A tail ending in -AAAG, -AAAGA, or -AAAGGA are effective in conferring stability against deadenylation. In some organisms, a GC sequence intervening a poly A sequence is shown to effectively show down 3′-5′ exonuclease mediated decay. A modification contemplated herein comprises an intervening non-A residue, or a non-A residue duplex intervening a poly A stretch at the 3′end.

In some embodiments, a triplex structure is introduced in the 3′ UTR which effectively stalls or slows down exonuclease activity involving the 3′ end.

In some embodiments, the mRNA with the modifications described above has an extended half-life and demonstrates stable expression over a longer period than the unmodified mRNA. In some embodiments, the mRNA stably expresses for greater than 2 days, 3 days, 4 days, 5 days, 6 days, 7 days, 8 days 9 days or 10 days or more, and the mRNA or its protein product is detectable in vivo. In some embodiments, the mRNA is detected up to 3 days, 4 days, 5 days, 6 days, 7 days, 8 days, 9 days, 10 days, 11 days, 12 days, 13 days, 14 days or 15 days in vivo. In some embodiments, a protein product of the mRNA is detected up to 3 days, 4 days, 5 days, 6 days, 7 days, 8 days, 9 days, 10 days, 11 days, 12 days, 13 days, 14 days, 15 days, 16 days, 17 days, 18 days, 20 days, 25 days or 30 days in vivo.

CircRNA and tectoRNA

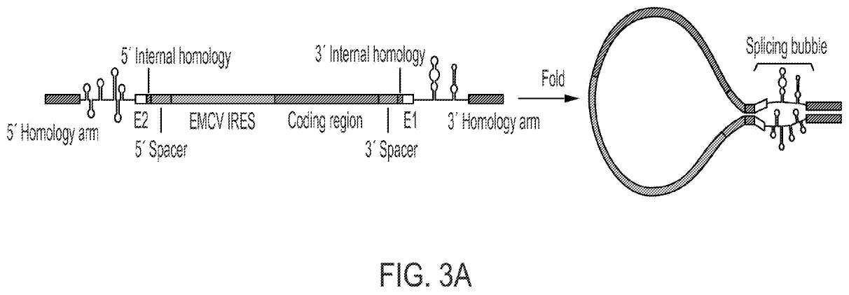

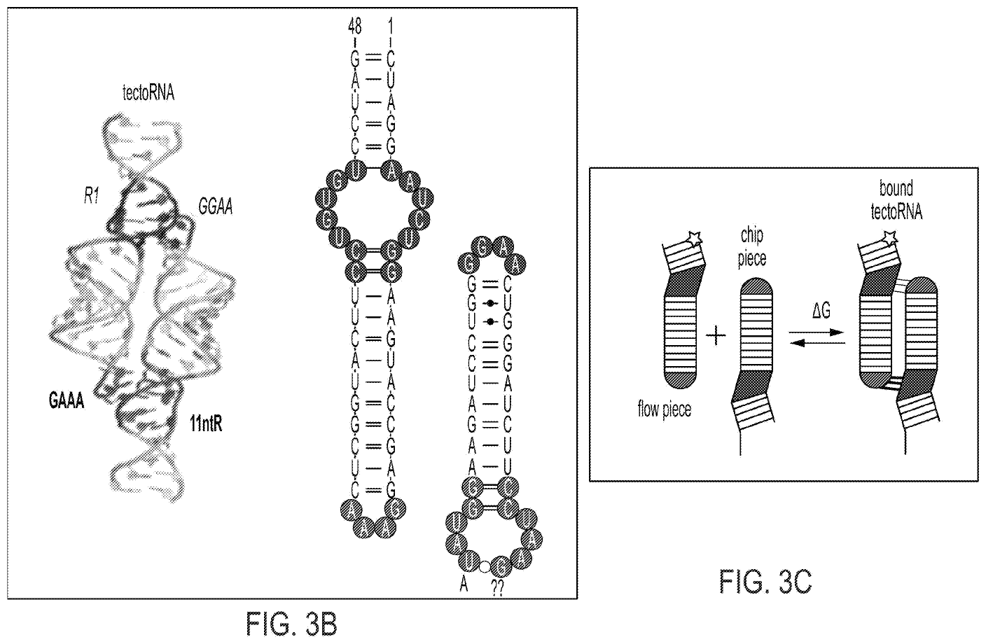

Circular RNA is useful in the design and production of stable form of RNA used as a messenger RNA to direct synthesis protein chains, such as long, multiply repeating protein chains. There are few methods to make circular RNA (circRNA). They include protein-mediated ligation of RNA ends using RNA ligase and using a split self-splicing intron, such that if the two halves of the intron are located and the ends of a transcribed mRNA, the intron will splice itself out and leave a ligated product (FIG. 3A).

Another technique relies on the ability of T4 DNA ligase to act as an RNA ligase when the RNA ends to be ligated are held together by an oligonucleotide. Both these techniques suffer from inefficiency and require a large amount of enzyme. A third technique uses the cyclization or circularization activity of group I introns where most of the intron sequences that carry out the reaction must remain a part of the circle.

Group I introns share a complex set of secondary and tertiary structures containing a series of conserved RNA stem loops which form the catalytic core. Many of these introns are self-splicing in vitro and can splice and form two ligated exons as RNA with no accessory protein factors. The products created by the group I autocatalytic reaction are (1) an upstream exon ligated at the 5′ splice site to the 3′ splice site of a downstream exon and (2) a linear intron that can undergo further reversible auto-catalysis to form a circular intron. The presence of such a large highly structured nucleic acid sequence severely limits the types of RNA sequences that can be made circular by that technique. In addition, the catalytic activity of the intron may remain and interfere with structure and function of the circular RNA.

It is useful to increase the rate of the reaction, and thus the overall efficiency, by bringing the ends of the RNA in closer proximity. Previous work has achieved this by including complementary RNA sequences 3′ and 5′ to the ends of the mRNA such that upon hybridization of these sequences, the ends of the mRNA are in closer proximity such that it can undergo the ligation or self-splicing reaction with an overall faster rate compared to without the complementary sequences. These are called homology arms (FIG. 3A) of the self-splicing version of the circularization reaction. A major issue with such hybridization strategy is that if there are complementary sequences within the coding region to either of the homology arms, hybridization would actually inhibit the splicing reaction and the arms would need to be optimized for each new coding region. An alternative to this strategy described herein is the use of RNA sequences that fold a three-dimensional structure to form a stable binding interaction that is independent of sequence.

Non-Watson-Crick RNA tertiary interactions can be exploited to construct ‘tectoRNA’ molecular units, defined as RNA molecules capable of self-assembly. The use of such type of tertiary interactions allows one to control and modulate the assembly process by manipulating cation concentration (e.g. Mg”), and/or suitable temperature and employing modularly designed ‘selector’ RNA molecules. For the self-assembly of one-dimensional arrays, a basic modular unit was designed that comprises a 4-way junction with an interacting module on each helical arm. In some embodiments, the interacting module is a GAAA loop or a specific GAAA loop receptor. Each tectoRNA can interact with two other tectoRNAs via the formation of four loop-receptor interactions, two with each partner molecule.

In some embodiments, the tectoRNA structures are suitably selected, and integrated in the RNA comprising the exon and intron to form a circRNA. In some embodiments, the integration is done by well-known molecular biology techniques such as ligation. In some embodiments, the tectoRNA forms a stable structure at high temperatures. The tectoRNA structure do not compete with internal RNA sequences, thereby creating high efficiency circularization and splicing.

The circRNA can comprise a coding sequence described in any of the preceding sections. For example, it can comprise a sequence encoding fusion protein comprising a tethering or a receptor molecule. The receptor can be a phagocytic receptor fusion protein.

In some embodiments, the intron is a self-splicing intron.

In some embodiments, the terminal regions having the tertiary structures, also termed scaffolding regions for the circRNA, are about 30 nucleotides to about 100 nucleotides long. In some embodiments, the tertiary structure motif is about 45 nucleotides, about 50 nucleotides, about 55 nucleotides, about 60 nucleotides, about 65 nucleotides, about 70 nucleotides or about 75 nucleotides long. In some embodiments, the tertiary motifs are formed at high temperatures. In some embodiments, the tertiary motifs are stable.

In some embodiments, the nucleic acid construct having the one or more modifications as described herein and comprising one or more sequences encoding one or more proteins or polypeptides, is stable when administered in vivo. In some embodiments, the nucleic acid is an mRNA. In some embodiments, the mRNA comprising one or more sequences encoding one or more proteins or polypeptides is stable in vivo for more than 2 days, for more than 3 days, more than 4 days, more than 5 days, more than 6 days, more than 7 days, more than 8 days, more than 9 days, more than 10 days, more than 11 days, more than 12 days, more than 13 days, more than 14 days, more than 15 days, more than 16 days, more than 17 days, more than 18 days, more than 19 days, or more than 20 days. In some embodiments, the protein encoded by the sequences in the mRNA can be detected in vivo at greater than 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days. In some embodiments, the protein encoded by the sequences in the mRNA can be detected in vivo for about 7 days after the mRNA is administered. In some embodiments, the protein encoded by the sequences in the mRNA can be detected in vivo for about 14 days after the mRNA is administered. In some embodiments, the protein encoded by the sequences in the mRNA can be detected in vivo for about 21 days after the mRNA is administered. In some embodiments, the protein encoded by the sequences in the mRNA can be detected in vivo for about 30 days after the mRNA is administered. In some embodiments, the protein encoded by the sequences in the mRNA can be detected in vivo for more than about 30 days after the mRNA is administered.

In some aspects, enhancing nucleic acid uptake or incorporation within the cell is contemplated for enhancing expression of the retrotransposition. One of the methods include obtaining a homogenous population of cells to initiate incorporation of the nucleic acid, e.g. via transfection, in case of plasmid vector constructs, or via electroporation or any other means that may be used suitably to deliver a nucleic acid molecule into the cell. In some embodiments, cell cycle synchronization may be sought. Cell cycle synchronization may be accomplished by sorting cells for a certain common phenotype. In some embodiments, the cell population may be subjected to a treatment with a reagent that can stall cell cycle progression of all cells at a certain stage. Exemplary reagents can be found in commercial databases, such as www.tocris.com/cell-biology/cell-cycle-inhibitors, or www.sebt.com/browse/chemicals-Other-Chemicals-cell-cycle-arresting-compounds. For example, itraconazole or nocodazole inhibits cell cycle at Gi phase, or reagents that arrest cell cycle at GO/Gi phase, for example, 5-[(4-Ethylphenyl)methylene]-2-thioxo-4-thiazolidinone (compound 10058-F4) (Tocris Bioscience); or a G2M cell cycle blocker, such as AZD 5438 (chemical name, 4-[2-Methyl-1-(1-methylethyl)-1H-imidazol-5-yl]-N-[4-(methylsulfonyl)phenyl]-2-pyrimidinamine) which blocks cell cycle at G2M, Gi or S phases, to name a few. Cyclosporin, hydroxyurea, thymidine, are well known reagents that can cause cell cycle arrests.

Some reagents may irreversibly alter a cell state or may be toxic for the cells. Serum deprivation of cells for about 2-16 hours prior to electroporation or transfection, depending on the cell type, may also be an easy and reversible strategy for cell synchronization.

In some embodiments, retrotransposition efficiency may be increased by encouraging generation of DNA double stranded breaks to a cell that has been transfected with or electroporated with the retrotransposition constructs as described herein and/or modulating the DNA repair machinery.

Application of these techniques may be limited depending on end uses of the cell that would undergo the genetic manipulation ex vivo for stable incorporation of a nucleic acid sequence by this method. In some cases, use of such techniques may be contemplated where robust expression of the protein or transcript encoded by the incorporated nucleic acid is expected as an outcome for a determined period of time. Method of introducing double stranded breaks in a cell include subjecting the cell to controlled ionizing radiation of about 0.1 Gy or less for a short period.

In some embodiments, efficiency of LINE-1 mediated retrotransposition may be increased by treating the cell with small molecule inhibitors of DNA repair proteins to increase the window for the reverse transcriptase to act. Exemplary small molecule inhibitors of DNA repair proteins may be Benzamide (CAS 55-21-0), Olaparib (Lynparza) (CAS 763113-22-0), Rucaparib (Clovis -AG014699, PF-01367338 Pfizer), Niraparib (MK-827 Tesaro) CAS 1038915-60-4); Veliparib (ABT-888 Abbvie) (CAS 912444-00-9); Camptothecin (CPT) (CAS 7689-03-4); Irinotecan (CAS 100286-90-6); Topotecan (Hycamtin® GlaxoSmithKline) (CAS 123948-87-8); NSC 19630 (CAS 72835-26-8); NSC 617145 (CAS 203115-63-3); ML216 (CAS 1430213-30-1); 6-hydroxyDL-dopa (CAS 21373-30-8); D-103; D-G23; DIDS (CAS 67483-13-0); B02 (CAS 1290541-46-6); RI-1 (CAS 415713-60-9); RI-2 (CAS 1417162-36-7); Streptonigrin (SN) (CAS 3930-19-6).

### III. Nucleic Acid Cargo:

### A. Transpene

In one aspect the transgene or noncoding sequence that is the heterologous nucleic acid sequence to be inserted within the genome of a cell is delivered as an mRNA. The mRNA may comprise greater than about 100, 200, 300, 400, 500, 600, 700, 800, 900, 1000, 1100, 1200, 1300, 1400, 1500, 1600, 1700, 1800, 1900, 2000, 3000, 4000, 5000, 6000, 7000, 8000, 9000, 10,000 bases. In some embodiments, the mRNA may be more than 10,000 bases long. In some embodiments, the mRNA may be about 11,000 bases long.

In some embodiments, the mRNA may be about 12,000 bases long. In some embodiments, the mRNA comprises a transgene sequence that encodes a fusion protein. In some embodiments, the nucleic acid is delivered as a plasmid.

In some embodiments, the nucleic acid is delivered in the cell by transfection. In some embodiments, the nucleic acid is delivered in the cell by electroporation. In some embodiments, the transfection or electroporation is repeated more than once to enhance incorporation of the nucleic acid into the cell.

Contemplated herein are retrotransposon mediated stable integration of a recombinant nucleic acid encoding a phagocytic or tethering receptor (PR) fusion protein (CFP). In some embodiments, the CFPs comprise: a PR subunit comprising: a transmembrane domain, and an intracellular domain comprising an intracellular signaling domain; and an extracellular domain comprising an antigen binding domain specific to an antigen of a target cell; wherein the transmembrane domain and the extracellular domain are operatively linked.

In some embodiments, the nucleic acid comprises a sequence encoding a chimeric fusion protein (CFP), the CFP comprising an extracellular domain comprising a CD5 binding domain, and a transmembrane domain operatively linked to the extracellular domain. In some embodiments, the CD5 binding domain is a CD5 binding protein, such as an antigen binding fragment of an antibody, a Fab fragment, an scFv domain or an sdAb domain. In some embodiments, wherein the CD5 binding domain comprises an scFv comprising (i) a variable heavy chain (VH) sequence with at least 90% sequence identity to EIQLVQSGGGLVKPGGSVRISCAASGYTFTNYGMNWVRQAPGKGLEWMGWINTHTGEPTYAD SFKGRFTFSLDDSKNTAYLQINSLRAEDTAVYFCTRRGYDWYFDVWGQGTTVTV (SEQ ID NO: 1); and (ii) a variable light chain (VL) sequence with at least 90% sequence identity to DIQMTQSPSSLSASVGDRVTITCRASQDINSYLSWFQQKPGKAPKTLIYRANRLESGVPSRFSGSG SGTDYTLTISSLQYEDFGIYYCQQYDESPWTFGGGTKLEIK (SEQ ID NO: 2). In some embodiments, the CFP further comprises an intracellular domain, wherein the intracellular domain comprises one or more intracellular signaling domains, and wherein a wild-type protein comprising the intracellular domain does not comprise the extracellular domain. In some embodiments, the one or more intracellular signaling domains comprises a phagocytic signaling domain. In some embodiments, the phagocytosis signaling domain comprises an intracellular signaling domain derived from a receptor other than Megf10, MerTk, FcaR, and Bail. In some embodiments, the phagocytosis signaling domain comprises an intracellular signaling domain derived from FcγR, FcαR or FcεR. In some embodiments, the phagocytosis signaling domain comprises an intracellular signaling domain with at least 90% sequence identity to LYCRRLKIQVRKAAITSYEKSDGVYTGLSTRNQETYETLKHEKPP (SEQ ID NO: 67). In some embodiments, the one or more intracellular signaling domains further comprises a proinflammatory signaling domain. In some embodiments, the proinflammatory signaling domain comprises a P13-kinase (PI3K) recruitment domain. In some embodiments, the proinflammatory signaling domain comprises a sequence with at least 90% sequence identity to YEDMRGILYAAPQLRSIRGQPGPNHEEDADSYENM (SEQ ID NO: 4). In some embodiments, the proinflammatory signaling domain is derived from an intracellular signaling domain of CD40. In some embodiments, the proinflammatory signaling domain comprises a sequence with at least 90% sequence identity to KVAKKPTNKAPHPKQEPQEINFPDDLPGSNTAAPVQETLHGCQPVTQEDGKESRISVQERQ (SEQ ID NO: 68). In some embodiments, the transmembrane domain comprises a CD8 transmembrane domain. In some embodiments, the transmembrane domain comprises a sequence with at least 90% sequence identity to IYIWAPLAGTCGVLLLSLVIT (SEQ ID NO: 6). In some embodiments, the extracellular domain further comprises a hinge domain derived from CD8, wherein the hinge domain is operatively linked to the transmembrane domain and the CD5 binding domain. In some embodiments, the extracellular domain comprises a sequence with at least 90% sequence identity to ALSNSIMYFSHFVPVFLPAKPTTTPAPRPPTPAPTIASQPLSLRPEACRPAAGGAVHTRGLD (SEQ ID NO: 7). In some embodiments, the CFP comprises an extracellular domain comprising a scFv that specifically binds CD5, and a hinge domain derived from CD8; a hinge domain derived from CD28 or at least a portion of an extracellular domain from CD68; a CD8 transmembrane domain, a CD28 transmembrane domain or a CD68 transmembrane domain; and an intracellular domain comprising at least two intracellular signaling domains, wherein the at least two intracellular signaling domains comprise: a first intracellular signaling domain derived from FcγR or FcεR, and a second intracellular signaling domain comprising a PI3K recruitment domain, or derived from CD40. In some embodiments, the recombinant polynucleic acid is an mRNA or circRNA. In some embodiments, the nucleic acid is delivered into a myeloid cell. In some embodiments, the nucleic acid is delivered into a CD14+ cell, a CD14+CD16—cell, an MO macrophage, an M2 macrophage, an M1 macrophage or a mosaic myeloid cell/macrophage. In some embodiments, the fusion protein comprises a sequence with at least 90% sequence identity to EIQLVQSGGGLVKPGGSVRISCAASGYTFTNYGMNWVRQAPGKGLEWMGWINTHTGEPTYAD SFKGRFTFSLDDSKNTAYLQINSLRAEDTAVYFCTRRGYDWYFDVWGQGTTVTVSSGGGGSGG GGSGGGGSDIQMTQSPSSLSASVGDRVTITCRASQDINSYLSWFQQKPGKAPKTLIYRANRLESG VPSRFSGSGSGTDYTLTISSLQYEDFGIYYCQQYDESPWTFGGGTKLEIKSGGGGSGALSNSIMYF SHFVPVFLPAKPTTTPAPRPPTPAPTIASQPLSLRPEACRPAAGGAVHTRGLDIYIWAPLAGTCGV LLLSLVITLYCRRLKIQVRKAAITSYEKSDGVYTGLSTRNQETYETLKHEKPPQGSGSYEDMRGI LYAAPQLRSIRGQPGPNHEEDADSYENM (SEQ ID NO: 69). In some embodiments, the fusion protein comprises a sequence with at least 90% sequence identity to

(SEQ ID NO: 70)
EIQLVQSGGGLVKPGGSVRISCAASGYTFTNYGMN
WVRQAPGKGLEWMGWINTHTGEPTYADSFKGRFTF
SLDDSKNTAYLQINSLRAEDTAVYFCTRRGYDWYF
DVWGQGTTVTVSSGGGGSGGGGSGGGGSDIQMTQS
PSSLSASVGDRVTITCRASQDINSYLSWFQQKPGK
APKTLIYRANRLESGVPSRFSGSGSGTDYTLTISS
LQYEDFGIYYCQQYDESPWTFGGGTKLEIKSGGGG
SGALSNSIMYFSHFVPVFLPAKPTTTPAPRPPTPA
PTIASQPLSLRPEACRPAAGGAVHTRGLDIYIWAP
LAGTCGVLLLSLVITLYCRLKIQVRKAAITSYEKS
DGVYTGLSTRNQETYETLKHEKPPQKKVAKKPTNK
APHPKQEPQEINFPDDLPGSNTAAPVQETLHGCQP
VTQEDGKESRISVQERQ
or
(SEQ ID NO: 71)
EIQLVQSGGGLVKPGGSVRISCAASGYTFTNYGMN
WVRQAPGKGLEWMGWINTHTGEPTYADSFKGRFTF
SLDDSKNTAYLQINSLRAEDTAVYFCTRRGYDWYF
DVWGQGTTVTVSSGGGGSGGGGSGGGGSDIQMTQS
PSSLSASVGDRVTITCRASQDINSYLSWFQQKPGK
APKTLIYRANRLESGVPSRFSGSGSGTDYTLTISS
LQYEDFGIYYCQQYDESPWTFGGGTKLEIKSGGGG
SGALSNSIMYFSHFVPVFLPAKPTTTPAPRPPTPA
PTIASQPLSLRPEACRPAAGGAVHTRGLDIYIWAP
LAGTCGVLLLSLVITLYCRRLKIQVRKAAITSYEK
SDGVYTGLSTRNQETYETLKHEKPPQKKVAKKPTN
KAPHPKQEPQEINFPDDLPGSNTAAPVQETLHGCQ
PVTQEDGKESRISVQERQ.

In some embodiments, the fusion protein is a transmembrane protein, an intracellular protein or an intracellular protein. In one embodiment the fusion protein is directed to enhancing the function of an immune cell, e.g., a myeloid cell, selected from monocyte, macrophages dendritic cells or precursors thereof. In one embodiment the fusion protein augments a cellular function of an immune cell, such as phagocytosis. The disclosure is not limited by the transgenes that can be expressed using the methods and compositions described. The transgenes indicated in this section are exemplary.

Provided herein are exemplary transgene candidates, for stable integration into the genome of a phagocytic cell. In one embodiment the transgene is a recombinant nucleic acid encoding a phagocytic receptor (PR) fusion protein (CFP). The recombinant nucleic acid has a PR subunit comprising: (i) a transmembrane domain, and (ii) an intracellular domain comprising a phagocytic receptor intracellular signaling domain; and an extracellular antigen binding domain specific to an antigen of a target cell; wherein the transmembrane domain and the extracellular antigen binding domain are operatively linked such that antigen binding to the target by the extracellular antigen binding domain of the fused receptor activated in the intracellular signaling domain of the phagocytic receptor. In some embodiments, the recombinant nucleic acid encodes a chimeric antigen receptor. In some embodiments, the chimeric antigen receptor is a chimeric antigen receptor (phagocytosis) (CAR-P). In some embodiments, the fusion protein is a recombinant protein for locking anti-phagocytic signals. In some embodiments, the fusion protein is a phagocytosis enhancing chimeric protein. In some embodiments, the chimeric protein has intracellular domains comprising active phagocytosis signal transduction domains. In some embodiments, the chimeric protein enhances the phagocytic potential by enhancing the inflammatory potential of the phagocytic cell in which it expresses. In some embodiments, the transgene is designed to express a chimeric protein which is activated by contact with an antigen in a target cell, whereupon the phagocytic cell phagocytoses the target cell and kills the target cell.

The terms “spacer” or “linker” as used in reference to a fusion protein refers to a peptide sequence that joins the protein domains of a fusion protein. Generally, a spacer has no specific biological activity other than to join or to preserve some minimum distance or other spatial relationship between the proteins or RNA sequences. However, in some embodiments, the constituent amino acids of a spacer can be selected to influence some property of the molecule such as the folding, net charge, or hydrophobicity of the molecule. Suitable linkers for use in an embodiment of the present disclosure are well known to those of skill in the art and include, but are not limited to, straight or branched-chain carbon linkers, heterocyclic carbon linkers, or peptide linkers. The linker is used to separate two antigenic peptides by a distance sufficient to ensure that, in some embodiments, each antigenic peptide properly folds. Exemplary peptide linker sequences adopt a flexible extended conformation and do not exhibit a propensity for developing an ordered secondary structure. Typical amino acids in flexible protein regions include Gly, Asn and Ser.

Virtually any permutation of amino acid sequences containing Gly, Asn and Ser would be expected to satisfy the above criteria for a linker sequence. Other near neutral amino acids, such as Thr and Ala, also can be used in the linker sequence.

The various exemplary proteins encoded by a transgene that can be expressed for enhancing the immune potential of a phagocytic cell are described below. This is not an exhaustive list but serves as an exemplary list for transgene design within the scope of the present disclosure.

In some embodiments, the PSP subunit comprises a transmembrane (TM) domain of a phagocytic receptor.

In some embodiments, the PSP subunit comprises an ICD domain of a phagocytic receptor.

In some embodiments, the ICD encoded by the recombinant nucleic acid comprises a domain selected from the group consisting of lectin, dectin 1, mannose receptor (CD206), scavenger receptor Al (SRA1), MARCO, CD36, CD163, MSR1, SCARA3, COLEC12, SCARA5, SCARBI, SCARB2, CD68, OLR1, SCARFI, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), and CD169 receptor.

In some embodiments, the ICD comprises the signaling domain derived from any one or more of: lectin, dectin 1, mannose receptor (CD206), scavenger receptor Al (SRA1), MARCO (Macrophage Receptor with Collagenous Structure, aliases: SRA6, SCARA2), CD36 (Thrombospondin receptor, aliases: Scavenger Receptor class B, member 3), CD163 (Scavenger receptor, cysteine rich-type 1), MSR1, SCARA3, COLEC12 (aliases: Scavenger Receptor With C-Type Lectin, SCARA4, or Collectin 12), SCARA5, SCARBI, SCARB2, CD68 (SCARD, microsialin), OLR1 (Oxidized Low Density Lipoprotein Receptor 1, LOX1, or C-Type Lectin Domain Family 8 Member A), SCARFI, SCARF2, SRCRB4D, SSC5D, and CD169 (aliases, Sialoadhesin receptor, SIGLEC1).

In some embodiments, the recombinant nucleic acid encodes, for example, an intracellular domain of human MARCO. The PSR subunit comprises an intracellular domain having a 44 amino acid ICD of human MARCO having an amino acid sequence: MRNKKILKEDELLSETQQAAFHQIAMEPFEINVPKPKRRNGVNF (SEQ ID NO: 72). In some embodiments, the PSR subunit comprises a variant which is at least 70%, 75%, 80%, 85%, 90% or 95% identical to the intracellular domain of MARCO.

In some embodiments, for example, the PSR (phagocytic scavenger receptor) comprises a transmembrane region of human MARCO.

In some embodiments, the recombinant nucleic acid encodes an intracellular domain of human SRA1. The PSR subunit comprises an intracellular domain having a 50 amino acid ICD of human SRA1 having an amino acid sequence: MEQWDHFHNQQEDTDSCSESVKFDARSMTA LLPPNPKNSPSLQEKLKSFK (SEQ ID NO: 73). In some embodiments, the PSR subunit comprises a variant which is at least 70%, 75%, 80%, 85%, 90% or 95% identical to the intracellular domain of human SRA1. The intracellular region of SRA has a phosphorylation site.

In some embodiments, the PSR comprises a transmembrane region of human SRA1.

In some embodiments, for example, the recombinant nucleic acid comprises an intracellular domain of CD36. In some embodiments, the recombinant nucleic acid comprises a TM domain of CD36. Naturally occurring full length CD36 has two TM domains and two short intracellular domains, and an extracellular domain of CD36 binds to oxidized LDL. Both of the intracellular domains contain pairs of cysteines that are fatty acid acylated. It lacks known signaling domains (e.g. kinase, phosphatase, g-protein binding, or scaffolding domains). N-terminal cytoplasmic domain is extremely short (5-7 amino acid residues) and is closely associated with the internal leaflet of the plasma membrane. The carboxy-terminal domain contains 13 amino acids, containing a CXCX5K motif homologous to a region in the intracellular domain of CD4 and CD8 that is known to interact with signaling molecules. The intracellular domain of CD36 is capable of assembling a signaling complex that activates lyn kinases, MAP kinases and Focal Adhesion Kinases (FAK), and inactivation of src homology 2-containing phosphotyrosine phosphatase (SHP-2). Members of the guanine nucleotide exchange factors (GEFs) have been identified as potential key signaling intermediates.

In some embodiments, the recombinant nucleic acid encodes for example, an intracellular domain of human SCARA3. In some embodiments, the PSR subunit comprises a variant which is at least 70%, 75%, 80%, 85%, 90% or 95% identical to the intracellular domain of human SCARA3. In some embodiments, the PSR comprises the TM domain of SCARA3. In some embodiments, the TM domains are about 20-30 amino acids long.

Scavenger receptors may occur as homo or hetero dimers. MARCO, for example occurs as a homo trimer.

In some embodiments, the TM domain or the ICD domain of the PSP is not derived from FcR, Megf10, Bail or MerTK. In some embodiments, the ICD of the PSR does not comprise a CD3 zeta intracellular domain.

In some embodiments, the intracellular domain and transmembrane domains are derived from FcR beta.

In one aspect the recombinant nucleic acid encodes a chimeric antigenic receptor for enhanced phagocytosis (CAR-P), which is a phagocytic scavenger receptor (PSR) fusion protein (CFP) comprising: (a) an extracellular domain comprising an extracellular antigen binding domain specific to an antigen of a target cell, (b) a transmembrane domain, and (c) a recombinant PSR intracellular signaling domain, wherein the recombinant PSR intracellular signaling domain comprises a first portion derived from a phagocytic and a second portion derived from non-phagocytic receptor.

In some embodiments, the second portion is not a PI3K recruitment domain. In some embodiments, the second portion is a PI3K recruitment domain.

The second portion derived from non-phagocytic receptor may comprise an intracellular signaling domain that enhances phagocytosis, and/or inflammatory potential of the engineered phagocytic cells expressing the recombinant nucleic acid. In some embodiments, the second portion derived from non-phagocytic receptor comprises more than one intracellular domain (ICD). In some embodiments, the second portion derived from non-phagocytic receptor comprises a second ICD. In some embodiments, the second portion derived from non-phagocytic receptor comprises a second and a third ICD. In some embodiments, the second portion derived from non-phagocytic receptor comprises a second, a third and a fourth ICD, wherein the second portion is encoded by the recombinant nucleic acid. The respective second portions comprising a second, or third or fourth ICD derived from non-phagocytic receptor are described as follows.

### Chimeric Antigen Receptors for Enhancing Intracellular Signaling and Inflammation Activation

In one aspect, the recombinant nucleic acid encodes a second intracellular domain in addition to the phagocytic ICD, which confers capability of potent pro-inflammatory immune activation, such as when macrophages engage in fighting infection. The second intracellular domain (second ICD) is fused to the cytoplasmic terminus of the first phagocytic ICD. The second intracellular domain provides a second signal is necessary to trigger inflammasomes and pro-inflammatory signals. Nod-like receptors (NLRs) are a subset of receptors that are activated in innate immune response, and oligomerize to form multi-protein complexes that serve as platforms to recruit proinflammatory caspases and induce their cleavage and activation. This leads to direct activation of ROS, and often result in a violent cell death known as pyroptosis. There are four inflammasome complexes, NLRPlm, NLRP3, IPAF and AIM2.

The tumor microenvironment (TME) constitutes an immunosuppressive environment. Influence of IL-10, glucocorticoid hormones, apoptotic cells, and immune complexes can interfere with innate immune cell function. Immune cells, including phagocytic cells settle into a tolerogenic phenotype. In macrophages, this phenotype, commonly known as the M2 phenotype is distinct from the M1 phenotype, where the macrophages are potent and capable of killing pathogens. Macrophages exposed to LPS or IFN-gamma, for example, can polarize towards an M1 phenotype, whereas macrophages exposed to IL-4 or IL-13 will polarize towards an M2 phenotype. LPS or IFN-gamma can interact with Toll-like receptor 4 (TLR4) on the surface of macrophages inducing the Trif and MyD88 pathways, inducing the activation of transcription factors IRF3, AP-1, and NFKB and thus activating TNFs genes, interferon genes, CXCL10, NOS2, IL-12, etc., which are necessary in a pro-inflammatory Ml macrophage response. Similarly, IL-4 and IL-13 bind to IL-4R, activation the Jak/Stat6 pathway, which regulates the expression of CCL17, ARGI, IRF4, IL-10, SOCS3, etc., which are genes associated with an anti-inflammatory response (M2 response). Expression of CD14, CD80, D206 and low expression of CD163 are indicators of macrophage polarization towards the M1 phenotype.

In some embodiments, the recombinant nucleic acid encodes one or more additional intracellular domains, comprising a cytoplasmic domain for inflammatory response. In some embodiments, expression of the recombinant nucleic acid encoding the phagocytic receptor (PR) fusion protein (CFP) comprising the cytoplasmic domain for inflammatory response in the engineered macrophages confers potent pro-inflammatory response similar to the M1 phenotype.

In some embodiments, the cytoplasmic domain for inflammatory response can be the signal transducing domains or regions of TLR3, 4, 9, MYD88, TRIF, RIG-1, MDA5, CD40, IFN receptor, NLRP-1-14, NOD1, NOD2, Pyrin, AIM2, NLRC4, CD40.

In some embodiments, the expression of the recombinant nucleic acid encoding the phagocytic scavenger receptor (PSR) fusion protein (CFP) comprises a pro-inflammatory cytoplasmic domain for activation of IL-1 signaling cascade.

In some embodiments, the cytoplasmic portion of the chimeric receptor (for example, phagocytic receptor (PR) fusion protein (CFP)) comprises a cytoplasmic domain from a toll-like receptor, such as the intracellular signaling domains of toll-like receptor 3 (TLR3), toll-like receptor 4 (TLR4), toll-like receptor 7 (TLR7), toll-like receptor 8 (TLR8), toll-like receptor 9 (TLR9). In some embodiments, the cytoplasmic portion of the chimeric receptor comprises a suitable region from interleukin-1 receptor-associated kinase 1 (IRAK1)._In some embodiments, the cytoplasmic portion of the chimeric receptor comprises a suitable region from differentiation primary response protein (MYD88)._In some embodiments, the cytoplasmic portion of the chimeric receptor comprises a suitable region from myelin and lymphocyte protein (MAL).

In some embodiments, the cytoplasmic portion of the chimeric receptor comprises a suitable region from retinoic acid inducible gene (RIG-1).

In some embodiments, the transmembrane domain of the PSR comprises the transmembrane domain of any one of MYD88, TLR3, TLR4, TLR7, TLR8, TLR9, MAL, IRAK1, proteins.

In some embodiments, the recombinant PSR intracellular signaling domain comprises a first portion derived from a phagocytic and a second portion derived from non-phagocytic receptor wherein the second portion derived from non-phagocytic receptor comprises a phosphorylation site. In some embodiments, the phosphorylation site comprises amino acid sequences suitable for an autophosphorylation site. In some embodiments, the phosphorylation site comprises amino acid sequences suitable phosphorylation by Src family kinases. In some embodiments, the phosphorylation site comprises amino acid sequences, which upon phosphorylation are capable of binding to SH2 domains in a kinase. In some embodiments, a receptor tyrosine kinase domain is fused at the cytoplasmic end of the CFP in addition to the first cytoplasmic portion. In some embodiments, the phosphorylation is a tyrosine phosphorylation.

In some embodiments, the second intracellular domain is an Immune receptor Tyrosine Activation Motif (ITAM). The ITAM motif is present in mammalian α and β immunoglobulin proteins, TCR γ receptors, FCR γ receptors subunits, CD3 chains receptors and NFAT activation molecule.

In some embodiments, the CFP intracellular domain comprises one ITAM motif. In some embodiments, the CFP intracellular domain comprises more than one ITAM motifs. In some embodiments, the CFP intracellular domain comprises two or more ITAM motifs. In some embodiments, the CFP intracellular domain comprises three or more ITAM motifs. In some embodiments, the CFP intracellular domain comprises four or more ITAM motifs. In some embodiments, the CFP intracellular domain comprises five or more ITAM motifs. In some embodiments, the CFP intracellular domain comprises six or more ITAM motifs. In some embodiments, the CFP intracellular domain comprises seven or more ITAM motifs. In some embodiments, the CFP intracellular domain comprises eight or more ITAM motifs. In some embodiments, the CFP intracellular domain comprises nine or more ITAM motifs. In some embodiments, the CFP intracellular domain comprises ten or more ITAM motifs.

In some embodiments, one or more domains in the first phagocytic ICD comprises a mutation.

In some embodiments, one or more domains in the second ICD comprises a mutation to enhance a kinase binding domain, to generate a phosphorylation site, to generate an SH2 docking site or a combination thereof.

### Co-Expression of an Inflammatory Gene

In one aspect, the recombinant nucleic acid comprises a coding sequence for a pro-inflammatory gene, which is co-expressed with the CFP in the engineered cell. In some embodiments, the pro-inflammatory gene is a cytokine. Examples include but not limited to TNF-, IL-la, IL-1P, IL-6, CSF, GMCSF, or IL-12 or interferons.

The recombinant nucleic acid encoding the proinflammatory gene can be monocistronic, wherein the two coding sequences for (a) the PSP and (b) the proinflammatory gene are post-transcriptionally or post-translationally cleaved for independent expression.

In some embodiments, the two coding sequences comprise a self-cleavage domain, encoding a P2A sequence, for example.

In some embodiments, the two coding regions are separated by an IRES site.

In some embodiments, the two coding sequences are encoded by a bicistronic genetic element.

The coding regions for (a) the PSP and (b) the proinflammatory gene can be unidirectional, where each is under a separate regulatory control. In some embodiments, the coding regions for both are bidirectional and drive in opposite directions. Each coding sequence is under a separate regulatory control.

Co-expression of the proinflammatory gene is designed to confer strong inflammatory stimulation of the macrophage and activate the surrounding tissue for inflammation.

### Integrin Activation Domains

Cell-cell and cell-substratum adhesion is mediated by the binding of integrin extracellular domains to diverse protein ligands; however, cellular control of these adhesive interactions and their translation into dynamic cellular responses, such as cell spreading or migration, requires the integrin cytoplasmic tails. These short tails bind to intracellular ligands that connect the receptors to signaling pathways and cytoskeletal networks (Calderwood DA, 2004, Integrin Activation, Journal of Cell Science 117, 657-666). Integrins are heterodimeric adhesion receptors formed by the non-covalent association of a and P subunits. Each subunit is a type I transmembrane glycoprotein that has relatively large extracellular domains and, with the exception of the P4 subunit, a short cytoplasmic tail. Individual integrin family members have the ability to recognize multiple ligands. Integrins can bind to a large number of extracellular matrix proteins (bone matrix proteins, collagens, fibronectins, fibrinogen, laminins, thrombospondins, vitronectin, and von Willebrand factor), reflecting the primary function of integrins in cell adhesion to extracellular matrices. Many “counter-receptors” are ligands, reflecting the role of integrins in mediating cell-cell interactions. Integrins undergo conformational changes to increase ligand affinity.

The Integrin P₂ subfamily consists of four different integrin receptors, α_{M}β₂ (CD11b/CD18, Mac-1, CR3, Mo-1), β_{L}β₂ (CD11a/CD18, LFA-1), α_{X}β₂ (CD11c/CD18), and α_{D}β₂ (CD11d/CD18). These leukocyte integrins are involved in virtually every aspect of leukocyte function, including the immune response, adhesion to and transmigration through the endothelium, phagocytosis of pathogens, and leukocyte activation.

The a subunits of all β₂ integrins contain an inserted region of ˜200 amino acids, termed the I or A domain. Highly conserved I domains are found in several other integrin a subunits and other proteins, such as certain coagulation and complement proteins. I domains mediate protein-protein interactions, and in integrins, they are integrally involved in the binding of protein ligands. Although the I domains dominate the ligand binding functions of their integrins, other regions of the a subunits do influence ligand recognition. As examples, in α_{M}β₂ a mAb (OKM1) recognizing an epitope outside the I domain but in the α_{M} subunit inhibits ligand binding; and the EF-hand regions in α_{L}β₂ and α₂β₁, integrins with I domains in their a subunits, contribute to ligand recognition. The α_{M} subunit, and perhaps other a subunits, contains a lectin-like domain, which is involved in engagement of non-protein ligands, and occupancy may modulate the function of the I domain.

As integrins lack enzymatic activity, signaling is instead induced by the assembly of signaling complexes on the cytoplasmic face of the plasma membrane. Formation of these complexes is achieved in two ways; first, by receptor clustering, which increases the avidity of molecular interactions thereby increasing the on-rate of binding of effector molecules, and second, by induction of conformational changes in receptors that creates or exposes effector binding sites. Within the ECM, integrins have the ability to bind fibronectin, laminins, collagens, tenascin, vitronectin and thrombospondin. Clusters of integrin/ECM interactions form focal adhesions, concentrating cytoskeletal components and signaling molecules within the cell. The cytoplasmic tail of integrins serve as a binding site for α-actinin and talin which then recruit vinculin, a protein involved in anchoring F-actin to the membrane. Talin is activated by kinases such as protein kinase C (PKCα).

Integrins are activated by selectins. Leucocytes express L-selectin, activated platelets express P-selectin, and activated endothelial cells express E- and P-selectin. P-selectin-mediated adhesion enables chemokine- or platelet-activating factor-triggered activation of P2 integrins, which stabilizes adhesion. It also facilitates release of chemokines from adherent leucocytes. The cytoplasmic domain of P-selectin glycoprotein ligand 1 formed a constitutive complex with Nef-associated factor 1. After binding of P-selectin, Src kinases phosphorylated Nef-associated factor 1, which recruit the phosphoinositide-3-OH kinase p85-p110δ heterodimer and result in activation of leukocyte integrins. E-selectin ligands transduce signals that also affect β2 integrin function. Selectins trigger activation of Src family kinases. SFKs activated by selectin engagement phosphorylate the immunoreceptor tyrosine-based activation motifs (ITAMs) in the cytoplasmic domains of DAP12 and FcRγ. In some respects, CD44 is sufficient to transduce signals from E-selectin. CD44 triggers the inside-out signaling of integrins. A final common step in integrin activation is binding of talin to the cytoplasmic tail of the β subunit. Kindlins, another group of cytoplasmic adaptors, bind to a different region of integrin β tails. Kindlins increase the clustering of talin-activated integrins. Kindlins are responsive to selectin signaling, however, kindlins are found mostly in hematopoietic cells, such as neutrophils. Selectin signaling as well as signaling upon integrin activation by chemokines components have shared components, including SFKs, Syk, and SLP-76.

In some embodiments, the intracellular domain of the recombinant PSR fusion protein comprises an integrin activation domain. The integrin activation domain comprises an intracellular domain of a selectin, for example, a P-selectin, L-selectin or E-selectin.

In some embodiments, the intracellular domain of the recombinant PSR fusion protein comprises an integrin activation domain of laminin.

In some embodiments, the intracellular domain of the recombinant PSR fusion protein comprises an integrin activation domain for activation of Talin.

In some embodiments, the intracellular domain of the recombinant PSR fusion protein comprises an integrin activation domain fused to the cytoplasmic end of the phagocytic receptor ICD domain.

### Chimeric Receptor for Enhancing Antigen Cross Presentation

In some embodiments, the recombinant nucleic acid encodes a domain capable of enabling cross presentation of antigens. In general, MHC class I molecules present self- or pathogen-derived antigens that are synthesized within the cell, whereas exogenous antigens derived via endocytic uptake are loaded onto MHC class II molecules for presentation to CD4+ T cells. MHC I-restricted presentation of endogenous antigens, in which peptides are generated by the proteasome. However, in some cases, DC can process exogenous antigens into the MHC-I pathway for presentation to CD8+ T cells. This is referred to as cross presentation of antigens. Soluble or exogenous antigenic components may get degraded by lysosomal proteases in the vacuoles and cross presented by DCs, instead of following the endocytic pathway. In some instances, chaperones, such as heat shock protein 90 (Hsp90) have shown to help cross present antigens by certain APCs. HSP-peptide complexes are known to be internalized by a distinct group of receptors compared to free polypeptides. These receptors are from the scavenger receptor families and included LOX-1, SREC-I/SCARF-I, and FEEL1/Stabilin-1. Both SREC-I and LOX-1 have been shown to mediate the cross presentation of molecular chaperone bound antigens and lead to activation of CD8⁺ T lymphocytes.

SREC-1 (scavenger receptor expressed by endothelial cells) has no significant homology to other types of scavenger receptors but has unique domain structures. It contains 10 repeats of EGF-like cysteine-rich motifs in the extracellular domain. Recently, the structure of SREC-I was shown to be similar to that of a transmembrane protein with 16 EGF-like repeats encoded by the Caenorhabditis elegans gene ced-I, which functions as a cell surface phagocytic receptor that recognizes apoptotic cells.

Cross presentation of cancer antigens through the Class-I MHC pathway results in enhanced CD8+ T cell response, which is associated with cytotoxicity and therefore beneficial in tumor regression.

In some embodiments, the intracellular domain of the CFP comprises a SREC1 intracellular domain. In some embodiments, the intracellular domain of the CFP comprises a SRECII intracellular domain.

In some embodiments, the PSR subunit comprises: an intracellular domain comprising a PSR intracellular signaling domain from SREC1 or SRECII.

In some embodiments, the PSR subunit comprises: (i) a transmembrane domain, and (ii) an intracellular domain comprising a PSR intracellular signaling domain from SREC1 or SRECII.

In some embodiments, the PSR subunit comprises: (i) a transmembrane domain, (ii) an intracellular domain comprising a PSR intracellular signaling domain, and (iii) an extracellular domain from SREC1 or SRECII.

### Transmembrane Domain of a CFP Fusion Protein

In some embodiments, the TM encoded by the recombinant nucleic acid comprises a domain of a scavenger receptor (SR). In some embodiments, the TM can be the TM domain of or derived from any one or more of: lectin, dectin 1, mannose receptor (CD206), SRA1, MARCO, CD36, CD163, MSR1, SCARA3, COLEC12, SCARA5, SCARBI, SCARB2, CD68, OLR1, SCARFI, SCARF2, SRCRB4D, SSC5D, and CD169.

In some embodiments, the TM domains are about 20-30 amino acids long. TM domains of SRs are about 20-30 amino acids long.

The TM domain or the ICD domain of the PSP is not derived from Megf10, Bail or MerTK. The ICD of the PSR does not comprise a CD3 zeta intracellular domain.

In some embodiments, the TM is derived from the same phagocytic receptor as the ICD.

In some embodiments, the TM region is derived from a plasma membrane protein. The TM can be selected from an Fc receptor (FcR). In some embodiments, nucleic acid sequence encoding domains from specific FcRs are used for cell-specific expression of a recombinant construct. An FCR-alpha region comprising the TM domain may be used for macrophage specific expression of the construct. FcRβ recombinant protein expresses in mast cells.

In some embodiments, the CFP comprises the TM of an FCR-beta (FcRβ).

In some embodiments, the CFP comprises both the FcRβ TM and ICD domains.

In some embodiments, the TM domain is derived from CD8.

In some embodiments, the TM is derived from CD2.

In some embodiments, the TM is derived from FCR alpha.

Extracellular domain of a CFP fusion protein

The extracellular domain comprises an antigen binding domain that binds to one or more target antigens on a target cell. The target binding domain is specific for the target. The extracellular domain can include an antibody or an antigen-binding domain selected from intrabodies, peptibodies, nanobodies, single domain antibodies. SMIPs, and multispecific antibodies.

In some embodiments, the extracellular domain includes a Fab binding domain. In yet other such embodiments, the extracellular domain includes a scFv.

In some embodiments, the chimeric antigen receptor comprises an extracellular antigen binding domain is derived from the group consisting of an antigen-binding fragment (Fab), a single-chain variable fragment (scFv), a nanobody, a VH domain, a VL domain, a single domain antibody (sdAb), a VNAR domain, and a VHH domain, a bispecific antibody, a diabody, or a functional fragment of any thereof. In some embodiments, the antigen-binding fragment (Fab), a single-chain variable fragment (scFv), a nanobody, a VH domain, a VL domain, a single domain antibody (sdAb), a VNAR domain, and a VHH domain, a bispecific antibody, a diabody, or a functional fragment of any thereof specifically bind to one or more antigens.

In some embodiments, the antigens are cancer antigens, and the target cell is a target cancer cell.

In some embodiments, the antigen for a target cancer cell is selected from the group consisting of CD3, CD4, CD5, CD7, CD19, CCR2, CCR4, CD30, CD37, TCRB1/2, TCR, TCR. CD22, HER2 (ERBB2/neu), Mesothelin, PSCA, CD123, CD30, CD171, CD138, CS-1, CLECLI, CD33, CD79b, EGFRvIII, GD2, GD3, BCMA, PSMA, RORI, FLT3, TAG72, CD38, CD44v6, CEA, EPCAM, B7H3 (CD276), KIT (CD 117), CD213A2, IL-1 IRa, PRSS21, VEGFR2, CD24, MUC-16, PDGFR-beta, SSEA-4, CD20, MUCI, EGFR, NCAM, Prostase, PAP, ELF2M, Ephrin B2, FAP, EphA2, GM3, TEM1/CD248, TEM7R, CLDN6, TSHR, GPRC5D, CD97, CD179a, ALK, and IGLL1.

Various cancer antigen targets can be selected from cancer antigens known to one of skill in the art. Depending on the cancer and the cell type involved cancer antigens are mutated native proteins. The antigen binding domains are screened for specificity towards mutated/cancer antigens and not the native antigens.

In some embodiments, for example, the cancer antigen for a target cancer cell can be one or more of the mutated/cancer antigens: MUC16, CCAT2, CTAG1A, CTAG1B, MAGE Al, MAGEA2, MAGEA3, MAGE A4, MAGEA6, PRAME, PCA3, MAGE Ci, MAGEC2, MAGED2, AFP, MAGEA8, MAGE9, MAGEA11, MAGEA12, IL13RA2, PLAC1, SDCCAG8, LSP1, CT45A1, CT45A2, CT45A3, CT45A5, CT45A6, CT45A8, CT45A10, CT47A1, CT47A2, CT47A3, CT47A4, CT47A5, CT47A6, CT47A8, CT47A9, CT47A10, CT47All, CT47A12, CT47B1, SAGEl, and CT55.

In some embodiments, for example, the cancer antigen for a target cancer cell can be one or more of the mutated/cancer antigens: CD2, CD3, CD4, CD5, CD7, CD8, CD20, CD30, CD45, CD56, where the cancer is a T cell lymphoma.

In some embodiments, for example, the cancer antigen for a target cancer cell can be one or more of the mutated/cancer antigens: IDH1, ATRX, PRL3, or ETBR, where the cancer is a glioblastoma.

In some embodiments, for example, the cancer antigen for a target cancer cell can be one or more of the mutated/cancer antigens: CA125, beta-hCG, urinary gonadotropin fragment, AFP, CEA, SCC, inhibin or extradiol, where the cancer is ovarian cancer.

In some embodiments, the cancer antigen for a target cancer cell may be HER2.

In some embodiments, the cancer antigen for a target cancer cell may be EGFR Variant III.

In some embodiments, the cancer antigen for a target cancer cell may be CD19.

In some embodiments, the SR subunit region comprises an extracellular domain (ECD) of the scavenger receptor. In some embodiments, the ECD of the scavenger receptor comprises an ECD domain of the SR comprising the ICD and the TM domains. In some embodiments, the SR-ECD contributes to the binding of the phagocyte to the target cell, and in turn is activated, and activates the phagocytosis of the target cell.

In some embodiments, the PSR domain optionally comprises the ECD domain or portion thereof of the respective scavenger receptor the ICD and TM domains of which is incorporated in the PSR.

Therefore, in some embodiments, In some embodiments, the ECD encoded by the recombinant nucleic acid comprises a domain selected from the group consisting of lectin, dectin 1, mannose receptor (CD206), scavenger receptor A1 (SRA1), MARCO, CD36, CD163, MSR1, SCARA3, COLEC12, SCARA5, SCARBI, SCARB2, CD68, OLR1, SCARFI, SCARF2, CXCL16, STAB1, STAB2, SRCRB4D, SSC5D, CD205, CD207, CD209, RAGE, CD14, CD64, F4/80, CCR2, CX3CR1, CSF1R, Tie2, HuCRIg(L), and CD169 receptor. The extracellular domains of most macrophage scavenger receptors contain scavenger receptors with a broad binding specificity that may be used to discriminate between self and non-self in the nonspecific antibody-independent recognition of foreign substances. The type I and II class A scavenger receptors (SR-All and SR-All) are trimeric membrane glycoproteins with a small NH2-terminal intracellular domain, and an extracellular portion containing a short spacer domain, an α-helical coiled-coil domain, and a triple-helical collagenous domain. The type I receptor additionally contains a cysteine-rich COOH-terminal (SRCR) domain. These receptors are present in macrophages in diverse tissues throughout the body and exhibit an unusually broad ligand binding specificity. They bind a wide variety of polyanions, including chemically modified proteins, such as modified LDL, and they have been implicated in cholesterol deposition during atherogenesis. They may also play a role in cell adhesion processes in macrophage-associated host defense and inflammatory conditions.

In some embodiments, the SR ECD is designed to bind to pro-apoptotic cells. In some embodiments, the scavenger receptor ECD comprises a binding domain for a cell surface molecule of a cancer cell or an infected cell.

In some embodiments, the extracellular domain of the PR subunit is linked by a linker to a target cell binding domain, such as an antibody or part thereof, specific for a cancer antigen.

In some embodiments, the extracellular antigen binding domain comprises one antigen binding domain. In some embodiments, the extracellular antigen binding domain comprises more than one binding domain. In some embodiments, the binding domain is an scFv. In some embodiments, the binding domain is an single domain antibody (sdAb). In some embodiments, the binding domain is fused to the recombinant PR at the extracellular domain. In some embodiments, the binding domain (e.g., scFv) and the extracellular domain of the PR are linked via a linker.

In some embodiments, the ECD antigen binding domain can bind to an intracellular antigen. In some embodiments, the intracellular antigen is a cancer antigen.

In some embodiments, the extracellular antigen binding domain binds to the target ligand with an affinity of less than 1000 nM. In some embodiments, the extracellular antigen binding domain binds to the target ligand with an affinity of less than 500 nM. In some embodiments, the extracellular antigen binding domain binds to the target ligand with an affinity of less than 450 nM. In some embodiments, the extracellular antigen binding domain binds to the target ligand with an affinity of less than 400 nM. In some embodiments, the extracellular antigen binding domain binds to the target ligand with an affinity of less than 350 nM. In some embodiments, the extracellular antigen binding domain binds to the target ligand with an affinity of less than 250 nM. In some embodiments, the extracellular antigen binding domain binds to the target ligand with an affinity of less than 200 nM. In some embodiments, the extracellular antigen binding domain binds to the target ligand with an affinity of less than 100 nM. In some embodiments, the extracellular antigen binding domain binds to the target ligand with an affinity ranging between than 200 nM to 1000 nM. In some embodiments, the extracellular antigen binding domain binds to the target ligand with an affinity ranging between than 300 nM to 1.5 mM. In some embodiments, the antigen binding domain binds to the target ligand with an affinity>200 nM, >300 nM or >500 nM.

### Peptide Linker

In some embodiments, the extracellular antigen binding domains, scFvs are linked to the TM domain or other extracellular domains by a linker. In some embodiments, where there are more than one scfv at the extracellular antigen binding domain the more than scfvs are linked with each other by linkers.

In some embodiments, the linkers are flexible. In some embodiments, the linkers comprise a hinge region. Linkers are usually short peptide sequences. In some embodiments, the linkers are stretches of Glycine and one or more Serine residues. Other amino acids preferred for short peptide linkers include but are not limited to threonine (Thr), serine (Ser), proline (Pro), glycine (Gly), aspartic acid (Asp), lysine (Lys), glutamine (Gln), asparagine (Asn), and alanine (Ala) arginine (Arg), phenylalanine (Phe), glutamic acid (Glu). Of these Pro, Thr, and Gln are frequently used amino acids for natural linkers. Pro is a unique amino acid with a cyclic side chain which causes a very restricted conformation. Pro-rich sequences are used as interdomain linkers, including the linker between the lipoyl and E3 binding domain in pyruvate dehydrogenase (GA₂PA₃PAKQEA₃PAPA₂KAEAPA₃PA₂KA (SEQ ID NO: 75)). For the purpose of the disclosure, the empirical linkers may be flexible linkers, rigid linkers, and cleavable linkers. Sequences such as (G4S)x (where x is multiple copies of the moiety, designated as 1, 2, 3, 4, and so on) (SEQ ID NO: 76) comprise a flexible linker sequence. Other flexible sequences used herein include several repeats of glycine, e.g., (Gly)6 (SEQ ID NO: 77) or (Gly)8 (SEQ ID NO: 78). On the other hand, a rigid linker may be used, for example, a linker (EAAAK)x, where x is an integer, 1, 2, 3, 4 etc. (SEQ ID NO: 79) gives rise to a rigid linker.

In some embodiments, the linker comprises at least 2, or at least 3 amino acids. In some embodiments, the linker comprises 4 amino acids. In some embodiments, the linker comprises 5 amino acids. In some embodiments, the linker comprises 6 amino acids. In some embodiments, the linker comprises 7 amino acids. In some embodiments, the linker comprises 8 amino acids. In some embodiments, the linker comprises 9 amino acids. In some embodiments, the linker comprises 8 amino acids. In some embodiments, the linker comprises 10 amino acids. In some embodiments, the linker comprises 11 amino acids. In some embodiments, the linker comprises 12 amino acids. In some embodiments, the linker comprises 13 amino acids. In some embodiments, the linker comprises 14 amino acids. In some embodiments, the linker comprises 15 amino acids. In some embodiments, the linker comprises 16 amino acids. In some embodiments, the linker comprises 17 amino acids. In some embodiments, the linker comprises 18 amino acids. In some embodiments, the linker comprises 19 amino acids. In some embodiments, the linker comprises 20 amino acids.

As contemplated herein, any suitable ECD, TM or ICD domain can be cloned interchangeably in the suitable portion of any one of the CARP receptors described in the disclosure to obtain a protein with enhanced phagocytosis compared to an endogenous receptor.

### Characteristics of the Fusion Proteins:

The CFP can structurally incorporate into the cell membrane of the cell in which it is expressed.

Specific leader sequences in the nucleic acid construct, such as the signal peptide can be used to direct plasma membrane expression of the encoded protein. The transmembrane domain encoded by the construct can incorporate the expressed protein in the plasma membrane of the cell.

In some embodiments, the transmembrane domain comprises a TM domain of an FcRalpha receptor, which dimerizes with endogenous FcR-gamma receptors in the macrophages, ensuring macrophage specific expression.

The CFP can render the cell that expresses it as potently phagocytic. When the recombinant nucleic acid encoding the CFP is expressed in a cell, the cell can exhibit an increased phagocytosis of a target cell having the antigen of a target cell, compared to a cell not expressing the recombinant nucleic acid. When the recombinant nucleic acid is expressed in a cell, the cell can exhibit an increased phagocytosis of a target cell having the antigen of a target cell, compared to a cell not expressing the recombinant nucleic acid. In some embodiments, the recombinant nucleic acid when expressed in a cell, the cell exhibits at least 2-fold increased phagocytosis of a target cell having the antigen of a target cell, compared to a cell not expressing the recombinant nucleic acid. In some embodiments, the recombinant nucleic acid when expressed in a cell, the cell exhibits at least 3-fold, 4-fold, 5-fold, 6-fold, 7-fold, 8-fold, 9-fold, 10-fold, 20-fold 30-fold or at least 5-fold increased phagocytosis of a target cell having the antigen of a target cell, compared to a cell not expressing the recombinant nucleic acid.

In some embodiments, expression of SIRP-AICD enhances phagocytosis of the cell expressing it by 1.1 fold or more, 1.2 fold or more, 1.3 fold or more, q.4 fold or more, 1.5 fold or more, by 1.6 fold or more, 1.7 fold or more, 1.8 fold or more, 1.9 fold or more, 2 fold or more, 3 fold or more, 4 fold or more, 5 fold or more, 8 fold or more, 10 fold or more, 15 fold or more, 20 fold or more, 30 fold or more, 40 fold or more, 50 fold or more, 60 fold or more, 70 fold or more 80 fold or more, 90 fold or more, 100 fold or more, compared to a cell not expressing SIRP-AICD.

In some embodiments, the cells co-expressing SIRP-AICD and a CFP encoding a phagocytic receptor as described herein exhibits an augmented phagocytosis compared to a cell that does not express either of the proteins. In some embodiments, co-expressing SIRP-AICD and a CFP encoding a phagocytic receptor as described herein exhibits more than 2-fold, more than 3-fold, more than 4-fold, more than 5-fold, more than 6-fold, more than 7-fold, more than 8-fold, more than 9-fold, more than 10-fold, more than 20-fold, more than 30-fold, more than 40-fold, more than 50-fold, more than 60-fold, more than 70-fold, more than 80-fold, more than 90-fold, more than 100-fold, or more than 150-fold or more than 200-fold increase in phagocytic potential (measured in fold change of phagocytic index) compared to a cell that does not express either the SIRP-AICD or the CFP encoding a phagocytic receptor.

In some embodiments, expression of the any one of a CFP expressing a CD47 blocking extracellular domain of SIRPα and an intracellular domain of a phagocytic receptor augments phagocytic activity of a cell expressing it by at least 1.5 fold or more, 1.6 fold or more, 1.7 fold or more, 1.8 fold or more, 1.9 fold or more, 2 fold or more, 3 fold or more, 4 fold or more, 5 fold or more, 8 fold or more, 10 fold or more, 15 fold or more, 20 fold or more, 30 fold or more, 40 fold or more, 50 fold or more, 60 fold or more, 70 fold or more 80 fold or more, 90 fold or more, 100 fold or more, compared to a cell not expressing the CFP, or compared to a cell expressing SIRP-AICD.

In some embodiments, the enhancement in phagocytosis of target cells by a cell expressing either SIRP-AICD is highly increased compared to a phagocytic cell not expressing SIRP-AICD.

In some embodiments, the enhancement in phagocytosis of target cells by a cell expressing a CFP comprising a CD47 blocking extracellular domain of SIRPα and an intracellular domain of a phagocytic receptor is highly increased compared to a control phagocytic cell not expressing the fusion protein or a control phagocytic cell expressing the SIRP-ΔICD.

In some embodiments, when the recombinant nucleic acid described herein is expressed in a cell, the cell exhibits an increased cytokine production. The cytokine can comprise any one of: IL-1, IL-6, IL-12, IL-23, TNF, CXCL9, CXCL10, CXCL11, IL-18, IL-23, IL-27 and interferons.

In some embodiments, when the recombinant nucleic acid described herein is expressed in a cell, the cell exhibits an increased cell migration.

In some embodiments, when the recombinant nucleic acid described herein is expressed in a cell, the cell exhibits an increased immune activity. In some embodiments, when the recombinant nucleic acid is expressed in a cell, the cell exhibits an increased expression of MHC II. In some embodiments, when the recombinant nucleic acid is expressed in a cell, the cell exhibits an increased expression of CD80. In some embodiments, when the recombinant nucleic acid is expressed in a cell, the cell exhibits an increased expression of CD86. In some embodiments, when the recombinant nucleic acid is expressed in a cell, the cell exhibits an increased iNOS production.

In some embodiments, when the recombinant nucleic acid is expressed in a cell, the cell exhibits decreased trogocytosis of a target cell expressing the antigen of a target cell compared to a cell not expressing the recombinant nucleic acid.

In embodiments, the chimeric receptors may be glycosylated, pegylated, and/or otherwise post-translationally modified. In further embodiments, glycosylation, pegylation, and/or other posttranslational modifications may occur in vivo or in vitro and/or may be performed using chemical techniques. In additional embodiments, any glycosylation, pegylation and/or other posttranslational modifications may be N-linked or O-linked. In embodiments any one of the chimeric receptors may be enzymatically or functionally active such that, when the extracellular domain is bound by a ligand, a signal is transduced to polarize a macrophage.

In some embodiments, the chimeric fusion protein (CFP) comprises an extracellular domain (ECD) targeted to bind to CD5 (CD5 binding domain), for example, comprising a heavy chain variable region (VH) having an amino acid sequence as set forth in SEQ ID NO: 1. In some embodiments, the chimeric CFP comprises a CD5 binding heavy chain variable domain comprising an amino acid sequence that has at least 80%, 85%, 90%, 95%, 96%, 97%, 98%, 99% sequence identity to SEQ ID NO: 1. In some embodiments, the extracellular domain (ECD) targeted to bind to CD5 (CD5 binding domain) comprises a light chain variable domain (V_{L}) having an amino acid sequence as set forth in SEQ ID NO: 2. In some embodiments, the chimeric CFP comprises a CD5 binding light chain variable domain comprising an amino acid sequence that has at least 80%, 85%, 90%, 95%, 96%, 97%, 98%, 99% sequence identity to SEQ ID NO: 2.

In some embodiments, the CFP comprises an extracellular domain targeted to bind to HER2 (HER2 binding domain) having for example a heavy chain variable domain amino acid sequence as set forth in SEQ ID NO: 8 and a light chain variable domain amino acid sequence as set forth in SEQ ID NO: 9. In some embodiments, the CFP comprises a HER2 binding heavy chain variable domain comprising an amino acid sequence that has at least 80%, 85%, 90%, 95%, 96%, 97%, 98%, 99% sequence identity to SEQ ID NO: 8. In some embodiments, the CFP comprises a HER2 binding light chain variable domain comprising an amino acid sequence that has at least 80%, 85%, 90%, 95%, 96%, 97%, 98%, 99% sequence identity to SEQ ID NO: 9.

In some embodiments, the CFP comprises a hinge connecting the ECD to the transmembrane (TM). In some embodiments the hinge comprises the amino acid sequence of the hinge region of a CD8 receptor. In some embodiments, the CFP may comprise a hinge having the amino acid sequence set forth in SEQ ID NO: 7 (CD8a chain hinge domain). In some embodiments, the PFP hinge region comprises an amino acid sequence that is at least 80%, 85%, 90%, 95%, 96%, 97%, 98%, 99% sequence identity to SEQ ID NO: 7.

In some embodiments, the CFP comprises a CD8 transmembrane region, for example having an amino acid sequence set forth in SEQ ID NO: 6. In some embodiments, the CFP TM region comprises an amino acid sequence that is at least 80%, 85%, 90%, 95%, 96%, 97%, 98%, 99% sequence identity to SEQ ID NO: 6.

In some embodiments, the CFP comprises an intracellular domain having an FcR domain. In some embodiments, the CFP comprises an FcR domain intracellular domain comprises an amino acid sequence set forth in SEQ ID NO: 3, or at least a sequence having 80%, 85%, 90%, 95%, 96%, 97%, 98%, 99% sequence identity to SEQ ID NO: 3.

In some embodiments, the CFP comprises an intracellular domain having a PI3K recruitment domain. In some embodiments the PI3K recruitment domain comprises an amino sequence set forth in SEQ ID NO: 4. In some embodiments the PI3K recruitment domain comprises an amino acid sequence that has at least 80%, 85%, 90%, 95%, 96%, 97%, 98%, 99% sequence identity to SEQ ID NO: 4.

In some embodiments, the CFP comprises an intracellular domain having a CD40 intracellular domain. In some embodiments the CD40 ICD comprises an amino sequence set forth in SEQ ID NO: 5. In some embodiments the CD40 ICD comprises an amino acid sequence that has at least 80%, 85%, 90%, 95%, 96%, 97%, 98%, 99% sequence identity to SEQ ID NO: 5.

In some embodiments, the CD5 binding domain comprises an scFv comprising: (i) a variable heavy chain (VH) sequence of SEQ ID NO: 1 or with at least 90% sequence identity to SEQ ID NO: 1; and (ii) a variable light chain (V_{L}) sequence of SEQ ID NO: 2 or with at least 70%, 75%, 80%, 85%, 90%, 91%, 92%, 93%, 94%, 95%, 96%, 97%, 98% or 99% sequence identity to SEQ ID NO: 2. In some embodiments, the CD5 binding domain comprises an scFv comprising SEQ ID NO: 33 or with at least 70%, 75%, 80%, 85%, 90%, 91%, 92%, 93%, 94%, 95%, 96%, 97%, 98% or 99% sequence identity to SEQ ID NO: 33. In some embodiments, the HER2 binding domain comprises an scFv comprising: (i) a variable heavy chain (VH) sequence of SEQ ID NO: 8 or with at least 70%, 75%, 80%, 85%, 90%, 91%, 92%, 93%, 94%, 95%, 96%, 97%, 98% or 99% sequence identity to SEQ ID NO: 8; and (ii) a variable light chain (V_{L}) sequence of SEQ ID NO: 9 or with at least 70%, 75%, 80%, 85%, 90%, 91%, 92%, 93%, 94%, 95%, 96%, 97%, 98% or 99% sequence identity to SEQ ID NO: 9. In some embodiments, the CD5 binding domain comprises an scFv comprising SEQ ID NO: 32 or with at least 70%, 75%, 80%, 85%, 90%, 91%, 92%, 93%, 94%, 95%, 96%, 97%, 98% or 99% sequence identity to SEQ ID NO: 32. In some embodiments, the CFP further comprises an intracellular domain, wherein the intracellular domain comprises one or more intracellular signaling domains, and wherein a wild-type protein comprising the intracellular domain does not comprise the extracellular domain.

In some embodiments, the extracellular domain further comprises a hinge domain derived from CD8, wherein the hinge domain is operatively linked to the transmembrane domain and the anti-CD5 binding domain. In some embodiments, the extracellular hinge domain comprises a sequence of SEQ ID NO: 7 or with at least 70%, 75%, 80%, 85%, 90%, 91%, 92%, 93%, 94%, 95%, 96%, 97%, 98% or 99% sequence identity to SEQ ID NO: 7.

In some embodiments, the CFP comprises an extracellular domain fused to a transmembrane domain of SEQ ID NO: 30 or with at least 70%, 75%, 80%, 85%, 90%, 91%, 92%, 93%, 94%, 95%, 96%, 97%, 98% or 99% sequence identity to SEQ ID NO: 30. In some embodiments, the CFP comprises an extracellular domain fused to a transmembrane domain of SEQ ID NO: 31 or with at least 70%, 75%, 80%, 85%, 90%, 91%, 92%, 93%, 94%, 95%, 96%, 97%, 98% or 99% sequence identity to SEQ ID NO: 31.

In some embodiments, the transmembrane domain comprises a CD8 transmembrane domain. In some embodiments, the transmembrane domain comprises a sequence of SEQ ID NO: 6 or 29 or with at least 70%, 75%, 80%, 85%, 90%, 91%, 92%, 93%, 94%, 95%, 96%, 97%, 98% or 99% sequence identity to SEQ ID NO: 6 or 29. In some embodiments, the transmembrane domain comprises a sequence of SEQ ID NO: 18 or with at least 70%, 75%, 80%, 85%, 90%, 91%, 92%, 93%, 94%, 95%, 96%, 97%, 98% or 99% sequence identity to SEQ ID NO: 18. In some embodiments, the transmembrane domain comprises a sequence of SEQ ID NO: 34 or with at least 70%, 75%, 80%, 85%, 90%, 91%, 92%, 93%, 94%, 95%, 96%, 97%, 98% or 99% sequence identity to SEQ ID NO: 34. In some embodiments, the transmembrane domain comprises a sequence of SEQ ID NO: 19 or with at least 70%, 75%, 80%, 85%, 90%, 91%, 92%, 93%, 94%, 95%, 96%, 97%, 98% or 99% sequence identity to SEQ ID NO: 19.

In some embodiments, the CFP comprises one or more intracellular signaling domains that comprise a phagocytic signaling domain. In some embodiments, the phagocytosis signaling domain comprises an intracellular signaling domain derived from a receptor other than Megf10, MerTk, FcRα, and Bail. In some embodiments, the phagocytosis signaling domain comprises an intracellular signaling domain derived from a receptor other than Megf10, MerTk, an FcR, and Bail. In some embodiments, the phagocytosis signaling domain comprises an intracellular signaling domain derived from a receptor other than CD3(. In some embodiments, the phagocytosis signaling domain comprises an intracellular signaling domain derived from FcRγ, FcRα or FcRε. In some embodiments, the phagocytosis signaling domain comprises an intracellular signaling domain derived from CD3(. In some embodiments, the CFP comprises an intracellular signaling domain of any one of SEQ ID NOs: 3, 20, 27 and 28 or with at least 70%, 75%, 80%, 85%, 90%, 91%, 92%, 93%, 94%, 95%, 96%, 97%, 98% or 99% sequence identity to any one of SEQ ID NOs: 3, 20, 27 and 28. In some embodiments, the one or more intracellular signaling domains further comprises a proinflammatory signaling domain. In some embodiments, the proinflammatory signaling domain comprises a P13-kinase (PI3K) recruitment domain. In some embodiments, the proinflammatory signaling domain comprises a sequence of SEQ ID NO: 4 or with at least 70%, 75%, 80%, 85%, 90%, 91%, 92%, 93%, 94%, 95%, 96%, 97%, 98% or 99% sequence identity to SEQ ID NO: 4. In some embodiments, the proinflammatory signaling domain is derived from an intracellular signaling domain of CD40. In some embodiments, the proinflammatory signaling domain comprises a sequence of SEQ ID NO: 5 or with at least 70%, 75%, 80%, 85%, 90%, 91%, 92%, 93%, 94%, 95%, 96%, 97%, 98% or 99% sequence identity to SEQ ID NO: 5. In some embodiments, the CFP comprises an intracellular signaling domain of SEQ ID NO: 21 or with at least 70%, 75%, 80%, 85%, 90%, 91%, 92%, 93%, 94%, 95%, 96%, 97%, 98% or 99% sequence identity to SEQ ID NO: 21. In some embodiments, the CFP comprises an intracellular signaling domain of SEQ ID NO: 23 or with at least 70%, 75%, 80%, 85%, 90%, 91%, 92%, 93%, 94%, 95%, 96%, 97%, 98% or 99% sequence identity to SEQ ID NO: 23.

In some embodiments, the CFP comprises a sequence of SEQ ID NO: 14 or with at least 70%, 75%, 80%, 85%, 90%, 91%, 92%, 93%, 94%, 95%, 96%, 97%, 98% or 99% sequence identity to SEQ ID NO: 14. In some embodiments, the CFP comprises a sequence of SEQ ID NO: 15 or with at least 70%, 75%, 80%, 85%, 90%, 91%, 92%, 93%, 94%, 95%, 96%, 97%, 98% or 99% sequence identity to SEQ ID NO: 15. In some embodiments, the CFP comprises a sequence of SEQ ID NO: 16 or with at least 70%, 75%, 80%, 85%, 90%, 91%, 92%, 93%, 94%, 95%, 96%, 97%, 98% or 99% sequence identity to SEQ ID NO: 16. In some embodiments, the CFP comprises a sequence of SEQ ID NO: 24 or with at least 70%, 75%, 80%, 85%, 90%, 91%, 92%, 93%, 94%, 95%, 96%, 97%, 98% or 99% sequence identity to SEQ ID NO: 24. In some embodiments, the CFP comprises a sequence of SEQ ID NO:25 or with at least 70%, 75%, 80%, 85%, 70%, 75%, 80%, 85%, 90%, 91%, 92%, 93%, 94%, 95%, 96%, 97%, 98% or 99%, 91%, 92%, 93%, 94%, 95%, 96%, 97%, 98% or 99% sequence identity to SEQ ID NO: 25.

In some embodiments, the CFP comprises: (a) an extracellular domain comprising: (i) a scFv that specifically binds CD5, and (ii) a hinge domain derived from CD8; a hinge domain derived from CD28 or at least a portion of an extracellular domain from CD68; (b) a CD8 transmembrane domain, a CD28 transmembrane domain, a CD2 transmembrane domain or a CD68 transmembrane domain; and (c) an intracellular domain comprising at least two intracellular signaling domains, wherein the at least two intracellular signaling domains comprise: (i) a first intracellular signaling domain derived from FcRα, FcRγ or FcRε, and (ii) a second intracellular signaling domain: (A) comprising a P13K recruitment domain, or (B) derived from CD40. In some embodiments, the CFP comprises as an alternative (c) to the above: an intracellular domain comprising at least two intracellular signaling domains, wherein the at least two intracellular signaling domains comprise: (i) a first intracellular signaling domain derived from a phagocytic receptor intracellular domain, and (ii) a second intracellular signaling domain derived from a scavenger receptor phagocytic receptor intracellular domain comprising: (A) comprising a PI3K recruitment domain, or (B) derived from CD40. Exemplary scavenger receptors from which an intracellular signaling domain may be derived may be found in Table 2. In some embodiments, the CFP comprises and intracellular signaling domain derived from an intracellular signaling domain of an innate immune receptor.

In some embodiments, the recombinant polynucleic acid is an mRNA. In some embodiments, the recombinant polynucleic acid is a circRNA. In some embodiments, the recombinant polynucleic acid is a viral vector. In some embodiments, the recombinant polynucleic acid is delivered via a viral vector.

In some embodiments, the myeloid cell is a CD14+ cell, a CD14+/CD16—cell, a CD14+/CD16+ cell, a CD14-/CD16+ cell, CD14-/CD16—cell, a dendritic cell, an MO macrophage, an M2 macrophage, an M1 macrophage or a mosaic myeloid cell/macrophage/dendritic cell.

In one aspect, provided herein is a method of treating cancer in a human subject in need thereof comprising administering a pharmaceutical composition to the human subject, the pharmaceutical composition comprising: (a) a myeloid cell comprising a recombinant polynucleic acid sequence, wherein the polynucleic acid sequence comprises a sequence encoding a chimeric fusion protein (CFP), the CFP comprising: (i) an extracellular domain comprising an anti-CD5 binding domain, and (ii) a transmembrane domain operatively linked to the extracellular domain; and (b) a pharmaceutically acceptable carrier; wherein the myeloid cell expresses the CFP.

In some embodiments, upon binding of the CFP to CD5 expressed by a target cancer cell of the subject killing or phagocytosis activity of the myeloid cell is increased by greater than 20% compared to a myeloid cell not expressing the CFP. In some embodiments, growth of a tumor is inhibited in the human subject.

In some embodiments, the cancer is a CD5+ cancer. In some embodiments, the cancer is leukemia, T cell lymphoma, or B cell lymphoma. In some embodiments, the CFP comprises one or more sequences shown in Table A and/or Table B below.

TABLE A
Exemplary sequences of CFPs and domains thereof

| SEQ | | |
|---|---|---|
| ID NO | PFP/Domain | Sequence |
| 1 | Anti-CD5 heavy chain | EIQLVQSGGGLVKPGGSVRISCAASGYTFTNYGMNWV |
| | variable domain | RQAPGKGLEWMGWINTHTGEPTYADSFKGRFTFSLDD |
| | | SKNTAYLQINSLRAEDTAVYFCTRRGYDWYFDVWGQ |
| | | GTTVTV |
| 2 | Anti-CD5 light chain | DIQMTQSPSSLSASVGDRVTITCRASQDINSYLSWFQQK |
| | variable domain | PGKAPKTLIYRANRLESGVPSRFSGSGSGTDYTLTISSLQ |
| | | YEDFGIYYCQQYDESPWTFGGGTKLEIK |
| 33 | Anti-CD5 scFv | EIQLVQSGGGLVKPGGSVRISCAASGYTFTNYGMNWV |
| | | RQAPGKGLEWMGWINTHTGEPTYADSFKGRFTFSLDD |
| | | SKNTAYLQINSLRAEDTAVYFCTRRGYDWYFDVWGQ |
| | | GTTVTVSSGGGGSGGGGSGGGGSDIQMTQSPSSLSASV |
| | | GDRVTITCRASQDINSYLSWFQQKPGKAPKTLIYRANR |
| | | LESGVPSRFSGSGSGTDYTLTISSLQYEDFGIYYCQQYD |
| | | ESPWTFGGGTKLEIK |
| 3 | FcRγ-chain intracellular | LYCRRLKIQVRKAAITSYEKSDGVYTGLSTRNQETYET |
| | signaling domain | LKHEKPPQ |
| 20 | FcRγ-chain | LYCRLKIQVRKAAITSYEKSDGVYTGLSTRNQETYETL |
| | intracellular | KHEKPPQ |
| | signaling domain | |
| 27 | FcRγ-chain | RLKIQVRKAAITSYEKSDGVYTGLSTRNQETYETLKHE |
| | intracellular | KPPQ |
| | signaling domain | |
| 28 | FcRγ-chain | RLKIQVRKAAITSYEKSDGVYTGLSTRNQETYETLKHE |
| | intracellular | KPPQ |
| | signaling domain | |
| 4 | PI3K recruitment domain | YEDMRGILYAAPQLRSIRGQPGPNHEEDADSYENM |
| 5 | CD40 intracellular | KKVAKKPTNKAPHPKQEPQEINFPDDLPGSNTAAPVQE |
| | domain | TLHGCQPVTQEDGKESRISVQERQ |
| 6 | CD8α chain | IYIWAPLAGTCGVLLLSLVIT |
| | transmembrane domain | |
| 29 | CD8α chain | IYIWAPLAGTCGVLLLSLVITLYC |
| | transmembrane domain | |
| 7 | CD8α chain hinge | ALSNSIMYFSHFVPVFLPAKPTTTPAPRPPTPAPTIASQP |
| | domain | LSLRPEACRPAAGGAVHTRGLD |
| 8 | Anti-HER2 heavy chain | DIQMTQSPSSLSASVGDRVTITCRASQDVNTAVAWYQQ |
| | variable domain | KPGKAPKLLIYSASFLYSGVPSRFSGSRSGTDFTLTISSL |
| | | QPEDFATYYCQQHYTTPPTFGQGTKVEIKRTGSTSGSG |
| | | KPGSGEGSEVQLVE |
| 9 | Anti-HER2 light chain | LVQPGGSLRLSCAASGFNIKDTYIHWVRQAPGKGLEW |
| | variable domain | VARIYPTNGYTRYADSVKGRFTISADTSKNTAYLQMNS |
| | | LRAEDTAVYYCSRWGGDGFYAMDVWGQGTLVTV |
| 32 | Anti-HER2 scFv | DIQMTQSPSSLSASVGDRVTITCRASQDVNTAVAWYQQ |
| | | KPGKAPKLLIYSASFLYSGVPSRFSGSRSGTDFTLTISSL |
| | | QPEDFATYYCQQHYTTPPTFGQGTKVEIKRTGSTSGSG |
| | | KPGSGEGSEVQLVESSGGGGSGGGGSGGGGSLVQPGGS |
| | | LRLSCAASGFNIKDTYIHWVRQAPGKGLEWVARIYPTN |
| | | GYTRYADSVKGRFTISADTSKNTAYLQMNSLRAEDTA |
| | | VYYCSRWGGDGFYAMDVWGQGTLVTV |
| 17 | GMCSF Signal peptide | MWLQSLLLLGTVACSIS |
| 18 | CD28 transmembrane | FWVLVVVGGVLACYSLLVTVAFIIFWV |
| | domain | |
| 34 | CD2 Transmembrane | IYLIIGICGGGSLLMVFVALLVFYIT |
| | domain | |
| 19 | CD 68 transmembrane | ILLPLIIGLILLGLLALVLIAFCII |
| | domain | |
| 21 | TNFR1 intracellular | QRWKSKLYSIVCGKSTPEKEGELEGTTTKPLAPNPSFSP |
| | domain | TPGFTPTLGFSPVPSSTFTSSSTYTPGDCPNFAAPRREVA |
| | | PPYQGADPILATALASDPIPNPLQKWEDSAHKPQSLDTD |
| | | DPATLYAVVENVPPLRWKEFVRRLGLSDHEIDRLELQN |
| | | GRCLREAQYSMLATWRRRTPRREATLELLGRVLRDMD |
| | | LLGCLEDIEEALCGPAALPPAPSLLR |
| 22 | TNFR2 intracellular | PLCLQREAKVPHLPADKARGTQGPEQQHLLITAPSSSSS |
| | domain | SLESSASALDRRAPTRNQPQAPGVEASGAGEARASTGS |
| | | SDSSPGGHGTQVNVTCIVNVCSSSDHSSQCSSQASSTM |
| | | GDTDSSPSESPKDEQVPFSKEECAFRSQLETPETLLGSTE |
| | | EKPLPLGVPDAGMKPS |
| 23 | MDA5 intracellular | MSNGYSTDENFRYLISCFRARVKMYIQVEPVLDYLTFL |
| | domain | PAEVKEQIQRTVATSGNMQAVELLLSTLEKGVWHLGW |
| | | TREFVEALRRTGSPLAARYMNPELTDLPSPSFENAHDE |
| | | YLQLLNLLQPTLVDKLLVRDVLDKCMEEELLTIEDRNR |
| | | IAAAENNGNESGVRELLKRIVQKENWFSAFLNVLRQTG |
| | | NNELVQELTGSDCSESNAEIEN |
| 30 | CD8α chain hinge | ALSNSIMYFSHFVPVFLPAKPTTTPAPRPPTPAPTIASQP |
| | domain + transmembrane | LSLRPEACRPAAGGAVHTRGLDIYIWAPLAGTCGVLLL |
| | domain | SLVITLYC |
| 31 | CD8α chain hinge | ALSNSIMYFSHFVPVFLPAKPTTTPAPRPPTPAPTIASQP |
| | domain + transmembrane | LSLRPEACRPAAGGAVHTRGLDIYIWAPLAGTCGVLLL |
| | domain | SLVIT |
| 14 | CD5-FcRγ-PI3K | MWLQSLLLLGTVACSISEIQLVQSGGGLVKPGGSVRISC |
| | | AASGYTFTNYGMNWVRQAPGKGLEWMGWINTHTGEP |
| | | TYADSFKGRFTFSLDDSKNTAYLQINSLRAEDTAVYFC |
| | | TRRGYDWYFDVWGQGTTVTVSSGGGGSGGGGSGGGG |
| | | SDIQMTQSPSSLSASVGDRVTITCRASQDINSYLSWFQQ |
| | | KPGKAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQPLSL |
| | | RPEACRPAAGGAVHTRGLDIYIWAPLAGTCGVLLLSLV |
| | | ITLYCRRLKIQVRKAAITSYEKSDGVYTGLSTRNQETYE |
| | | TLKHEKPPQGSGSYEDMRGILYAAPQLRSIRGQPGPNHE |
| | | EDADSYENM |
| 15 | HER2-FcRγ-PD3K | MWLQSLLLLGTVACSISDIQMTQSPSSLSASVGDRVTIT |
| | | CRASQDVNTAVAWYQQKPGKAPKLLIYSASFLYSGVP |
| | | SRFSGSRSGTDFTLTISSLQPEDFATYYCQQHYTTPPTFG |
| | | QGTKVEIKRTGSTSGSGKPGSGEGSEVQLVESGGGLVQ |
| | | PGGSLRLSCAASGFNIKDTYIHWVRQAPGKGLEWVARI |
| | | YPTNGYTRYADSVKGRFTISADTSKNTAYLQMNSLRAE |
| | | DTAVYYCSRWGGDGFYAMDVWGQGTLVTVSSSGGGG |
| | | SGALSNSIMYFSHFVPVFLPAKPTTTPAPRPPTPAPTIAS |
| | | QPLSLRPEACRPAAGGAVHTRGLDIYIWAPLAGTCGVL |
| | | LLSLVITLYCRRLKIQVRKAAITSYEKSDGVYTGLSTRN |
| | | QETYETLKHEKPPQGSGSYEDMRGILYAAPQLRSIRGQP |
| | | GPNHEEDADSYENM |
| 16 | CD5-FcRγ-CD40 | MWLQSLLLLGTVACSISEIQLVQSGGGLVKPGGSVRISC |
| | | AASGYTFTNYGMNWVRQAPGKGLEWMGWINTHTGEP |
| | | TYADSFKGRFTFSLDDSKNTAYLQINSLRAEDTAVYFC |
| | | TRRGYDWYFDVWGQGTTVTVSSGGGGSGGGGSGGGG |
| | | SDIQMTQSPSSLSASVGDRVTITCRASQDINSYLSWFQQ |
| | | KPGKAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQPLSL |
| | | RPEACRPAAGGAVHTRGLDIYIWAPLAGTCGVLLLSLV |
| | | ITLYCRLKIQVRKAAITSYEKSDGVYTGLSTRNQETYET |
| | | LKHEKPPQKKVAKKPTNKAPHPKQEPQEINFPDDLPGS |
| | | NTAAPVQETLHGCQPVTQEDGKESRISVQERQ |
| 24 | CD5-FcRγ-MDA5 | MWLQSLLLLGTVACSISEIQLVQSGGGLVKPGGSVRISC |
| | | AASGYTFTNYGMNWVRQAPGKGLEWMGWINTHTGEP |
| | | TYADSFKGRFTFSLDDSKNTAYLQINSLRAEDTAVYFC |
| | | TRRGYDWYFDVWGQGTTVTVSSGGGGSGGGGSGGGG |
| | | SDIQMTQSPSSLSASVGDRVTITCRASQDINSYLSWFQQ |
| | | KPGKAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQPLSL |
| | | RPEACRPAAGGAVHTRGLDIYIWAPLAGTCGVLLLSLV |
| | | ITLYCRLKIQVRKAAITSYEKSDGVYTGLSTRNQETYET |
| | | LKHEKPPQGSGSMSNGYSTDENFRYLISCFRARVKMYI |
| | | QVEPVLDYLTFLPAEVKEQIQRTVATSGNMQAVELLLS |
| | | TLEKGVWHLGWTREFVEALRRTGSPLAARYMNPELTD |
| | | LPSPSFENAHDEYLQLLNLLQPTLVDKLLVRDVLDKCM |
| | | EEELLTIEDRNRIAAAENNGNESGVRELLKRIVQKENW |
| | | FSAFLNVLRQTGNNELVQELTGSDCSESNAEIEN |
| 25 | CD5-FcRγ-TNFR1 | MWLQSLLLLGTVACSISEIQLVQSGGGLVKPGGSVRISC |
| | | AASGYTFTNYGMNWVRQAPGKGLEWMGWINTHTGEP |
| | | TYADSFKGRFTFSLDDSKNTAYLQINSLRAEDTAVYFC |
| | | TRRGYDWYFDVWGQGTTVTVSSGGGGSGGGGSGGGG |
| | | SDIQMTQSPSSLSASVGDRVTITCRASQDINSYLSWFQQ |
| | | KPGKAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQPLSL |
| | | RPEACRPAAGGAVHTRGLDIYIWAPLAGTCGVLLLSLV |
| | | ITLYCRLKIQVRKAAITSYEKSDGVYTGLSTRNQETYET |
| | | LKHEKPPQGSGSQRWKSKLYSIVCGKSTPEKEGELEGT |
| | | TTKPLAPNPSFSPTPGFTPTLGFSPVPSSTFTSSSTYTPGD |
| | | CPNFAAPRREVAPPYQGADPILATALASDPIPNPLQKWE |
| | | DSAHKPQSLDTDDPATLYAVVENVPPLRWKEFVRRLG |
| | | LSDHEIDRLELQNGRCLREAQYSMLATWRRRTPRREAT |
| | | LELLGRVLRDMDLLGCLEDIEEALCGPAALPPAPSLLR |
| 26 | CD5-FcRγ-TNFR2 | MWLQSLLLLGTVACSISEIQLVQSGGGLVKPGGSVRISC |
| | | AASGYTFTNYGMNWVRQAPGKGLEWMGWINTHTGEP |
| | | TYADSFKGRFTFSLDDSKNTAYLQINSLRAEDTAVYFC |
| | | TRRGYDWYFDVWGQGTTVTVSSGGGGSGGGGSGGGG |
| | | SDIQMTQSPSSLSASVGDRVTITCRASQDINSYLSWFQQ |
| | | KPGKAPKTLIYRANRLESGVPSRFSGSGSGTDYTLTISSL |
| | | QYEDFGIYYCQQYDESPWTFGGGTKLEIKSGGGGSGAL |
| | | SNSIMYFSHFVPVFLPAKPTTTPAPRPPTPAPTIASQPLSL |
| | | RPEACRPAAGGAVHTRGLDIYIWAPLAGTCGVLLLSLV |
| | | ITLYCRLKIQVRKAAITSYEKSDGVYTGLSTRNQETYET |
| | | LKHEKPPQGSGSPLCLQREAKVPHLPADKARGTQGPEQ |
| | | QHLLITAPSSSSSSLESSASALDRRAPTRNQPQAPGVEAS |
| | | GAGEARASTGSSDSSPGGHGTQVNVTCIVNVCSSSDHS |
| | | SQCSSQASSTMGDTDSSPSESPKDEQVPFSKEECAFRSQ |
| | | LETPETLLGSTEEKPLPLGVPDAGMKPS |

TABLE B
Linker sequences

| SEQ ID | Sequence |
|---|---|
| 10 | SSGGGGSGGGGSGGGGS |
| 11 | SGGGGSG |
| 12 | SGGG |
| 13 | GSGS |

IV. Noncoding Exogenous Sequence for Delivery and Incorporation into the Genome of a Cell.

A noncoding sequence may be delivered into the cell and designed to be incorporated in the genome of the cell. The noncoding sequence as used herein, is a sequence that does not result in a translated protein product, but may have regulatory elements, such as transcribed products, such as inhibitory RNA. In some embodiments, such a sequence may be a miRNA sequence. In some embodiments, the sequence may be a sequence for siRNA generation. In some embodiments, the sequence may comprise an intronic sequence, or a binding site created such that one or more DNA binding proteins can dock on the site and influence the nature and behavior of the adjoining regions. In some embodiments, the sequence may be a transcription factor binding site. In some embodiments, the sequence may comprise an enhancer binding site. In some embodiments, the sequence may comprise a binding site for topoisomerase, gyrase, reverse transcriptase, polymerase, poly A binding protein, guanylyl cyclase, ligase, restriction enzymes, DNA methylase, HDAC enzymes, and many others. In some embodiments, the noncoding sequence may be directed to manipulating heterochromatin. A noncoding insert sequence, as it may also be referred to here, may be a few nucleotides to 5 kB in length.

### V. Plasmid Design and Recombinant Nucleic Acid Design Comprising an Insert Sequence

The nucleic acid construct comprising one or more sequences encoding one or more proteins or polypeptides is incorporated in a plasmid for transcription and generating an mRNA. mRNA can be transcribed in an in vitro system using synthetic system of cell extracts. Alternatively, mRNA can be generated in a cell and harvested. The cell can be a prokaryotic cell, such as a bacterial cell. In some embodiments, the cell is a eukaryotic cell. In some embodiments, the transcription occurs in a synthetic system. Provided herein are exemplary plasmid design.

In some embodiments, of the various aspects of the disclosure, a plasmid is designed for expression of the mRNA molecule comprising a heterologous sequence of interest that encodes a protein or a polypeptide. The plasmid comprises, inter alia, the sequences for genomic integration elements for integration of the heterologous sequence of interest that encodes a protein or a polypeptide; the sequence comprising the transgene or fragment thereof, operably linked to its separate promoter and regulatory elements that are required for its expression in the host following integration in the host genome, (such as, the subject who is administered the mRNA); one or more regulatory elements for transcription and generation of the mRNA including a promoter for expression of the mRNA, e.g. in a bacterial cell or cell extract, and 3′ stabilizing elements; sequences for one or more detection marker and/or selection markers.

As is known to one of skill in the art, a plasmid backbone can be an available vector, such as an in-house or commercially developed vector, that can be improved in various ways for best expression of the transcribed sequences, for example, (but not limited to), by introducing one or more desirable restriction digestion sites in the MCS (multiple cloning site), introducing a desired promoter for overall mRNA transcription, such as the T7 promoter, exchanging an existing sequence within the plasmid vector for one or more desired sequences, or introducing one or more desired segments, such as a selection marker sequence.

The plasmid comprises transcription regulatory elements, such as a promoter at the 5′ region, and a 3′-stabilizing element. In some embodiments, the promoter is chosen for enhanced mRNA transcription in the desired cell, such as an E coli bacterial cell. In some embodiments, the promoter for transcription of the plasmid is selected from a T7 promoter, a Sp6 promoter, pL (lambda) promoter, T3 promoter, trp promoter, araBad promoter, lac promoter or a Ptac promoter. In some embodiments, the promoter is a T7 promoter. T7 or Sp6 promoters are constitutive promoters and are useful for high level transcription or in vitro transcription. In some embodiments, the 3′ stabilizing element is a sequence from BGH 3′ element, WPRE 3′ element, SV40 element, hGH element and other elements. The 3′ element comprises the necessary poly A and transcription termination sequences.

Exemplary selection markers include antibiotic selection marker and/or expression detection marker. Antibiotic selection markers include but are not limited to ampicillin resistance gene sequence (beta lactamase gene or fragment thereof) conferring resistance to ampicillin, for example G418 selection marker, tetracycline resistance gene sequence conferring resistance to tetracycline, kanamycin resistance gene sequence conferring resistance to kanamycin, erythromycin resistance gene sequence conferring resistance to erythromycin, chloramphenicol resistance gene sequence conferring resistance to chloramphenicol, neomycin resistant gene sequence conferring resistance to neomycin, and others.

Exemplary expression detection marker include FLAG, HA, GFP and others.

In some embodiments, the and other tags that can be fused to one or more coding sequences to function as a surrogate for the expression of the desired protein or peptide to which it is fused.

In some embodiments, the plasmid is less than 20 kb in length. In some embodiments, the plasmid is less than 19 kb in length. In some embodiments, the plasmid is less than 20 kb in length. In some embodiments, the plasmid is less than 18 kb in length. In some embodiments, the plasmid is less than 20 kb in length. In some embodiments, the plasmid is less than 17 kb in length. In some embodiments, the plasmid is less than 20 kb in length. In some embodiments, the plasmid is less than 16 kb in length. In some embodiments, the plasmid is less than 15 kb in length. In some embodiments, the plasmid is less than 14 kb in length. In some embodiments, the plasmid is less than 13 kb in length. In some embodiments, the plasmid is less than 12 kb in length. In some embodiments, the plasmid is about 15 kb, about 14 kb, about 13 kb, about 12 kb or about 10 kb in length.

In some embodiments, the codon is optimized for maximized transcription suitable for the transcription system.

### VI. Features Related to the Expression of the Transgene In Vivo

### Transcription Regulatory Elements in the Recombinant Nucleic Acid Construct (Transgene)

In some embodiments, the recombinant nucleic comprises one or more regulatory elements within the noncoding regions that can be manipulated for desired expression profiles of the encoded proteins. In some embodiments, the noncoding region may comprise suitable enhancer. In some embodiments, the enhancer comprises a binding region for a regulator protein or peptide may be added to the cell or the system comprising the cell, for commencement of expression of the protein encoded under the influence of the enhancer. Conversely, a regulatory element may comprise a protein binding domain that remains bound with the cognate protein and continue to inhibit transcription and/or translation of recombinant protein until an extracellular signal is provided for the protein to decouple from the bound position to allow commencement of the protein synthesis. Examples include but are not limited to Tetracycline-inducible (Tet-Inducible or Tet-on) and Tetracycline repressible (Tet-off) systems known to one of skill in the art.

Construct comprising metabolic switch: In some embodiments, the 5′ and 3′ untranslated regions flanking the coding regions of the construct may be manipulated for regulation of expression of the recombinant protein encoded by the nucleic acid constructs described above. For instance, the 3′UTR may comprise one or more elements that are inserted for stabilizing the mRNA. In some embodiments, AU-Rich Elements (ARE) sequences are inserted in the 3′ UTR that result in binding of RNA binding proteins that stabilize or destabilize the mRNA, allowing control of the mRNA half-life.

In some embodiments, the 3′UTR may comprise a conserved region for RNA binding proteins (e.g. GAPDH) binding to mature mRNA strand preventing translation. In some embodiments, glycolysis results in the uncoupling of the RNA binding proteins (e.g. GAPDH) allowing for mRNA strand translation. The principle of the metabolic switch is to trigger expression of target genes when a cell enters a certain metabolic state. In resting cells, for example, GAPDH is an RNA binding protein (RBP). It binds to ARE sequences in the 3′UTR, preventing translation of mRNA. When the cell enters glycolysis, GAPDH is required to convert glucose into ATP, coming off the mRNA allowing for translation of the protein to occur. In some embodiments, the environment in which the cell comprising the recombinant nucleic acid is present, provides the metabolic switch to the gene expression. For example, hypoxic condition can trigger the metabolic switch inducing the disengaging of GAPDH from the mRNA. The expression of the mRNA therefore can be induced only when the macrophage leaves the circulation and enters into a tumor environment, which is hypoxic. This allows for systemic administration of the nucleic acid or a cell comprising the nucleic acid, but ensures a local expression, specifically targeting the tumor environment.

In some embodiments, the nucleic acid construct can be a split construct, for example, allowing a portion of the construct to be expressed under the control of a constitutive expression system whereas another portion of the nucleic acid is expressed under control of a metabolic switch, as described above. In some embodiments, the nucleic acid may be under bicistronic control. In some embodiments, the bicistronic vector comprises a first coding sequence under a first regulatory control, comprising the coding sequence of a target recognition moiety which may be under constitutive control; and a second coding sequence encoding an inflammatory gene expression which may be under the metabolic switch. In some embodiments, the bicistronic vector may be unidirectional. In some embodiments, the bicistronic vector may be bidirectional.

In some embodiments, the ARE sequences comprise protein binding motifs for binding ARE sequence that bind to ADK, ALDH18A1, ALDH6A1, ALDOA, ASS1, CCBL2, CS, DUT, ENOl, FASN, FDPS, GOT2, HADHB, HK2, HSD17B10, MDH2, NME1, NQ01, PKM2, PPP1CC, SUCLG1, TP11, GAPDH, or LDH.

### Pharmaceutical Compositions and Immunotherapy

In one aspect provided herein is a pharmaceutical composition comprising (i) the nucleic acid encoding the transgene is incorporated in a transpositioning or retrotranspositioning system comprising the transgene, the 5′- and 3′—flanking transposition or retrotranspositioning elements, the expression regulation elements, such as promoters, introns; and a nucleic acid encoding the transposase or retrotransposase, (ii) a nucleic acid delivery vehicle and a pharmaceutically acceptable salt or excipient.

In some embodiments, the pharmaceutical composition comprises cells comprising the nucleic acid encoding the transgene that is stably integrated in the genome of the cell and a pharmaceutically acceptable excipient. Nucleic acid constructs can be delivered with cationic lipids (Goddard, et al, Gene Therapy, 4:1231-1236, 1997; Gorman, et al, Gene Therapy 4:983-992, 1997; Chadwick, et al, Gene Therapy 4:937-942, 1997; Gokhale, et al, Gene Therapy 4:1289-1299, 1997; Gao, and Huang, Gene Therapy 2:710-722, 1995), using viral vectors (Monahan, et al, Gene Therapy 4:40-49, 1997; Onodera, et al, Blood 91:30-36, 1998), by uptake of “naked DNA”, and the like. Techniques well known in the art for the transformation of cells (see discussion above) can be used for the ex vivo administration of nucleic acid constructs. The exact formulation, route of administration and dosage can be chosen empirically. (See e.g. Fingl et al., 1975, in “The Pharmacological Basis of Therapeutics”, Ch. 1 pl).

In some embodiments, the nucleic acid comprising the transgene and the transposable elements is introduced or incorporated in the cell by known methods of nucleic acid transfer inside a cell, such as using lipofectamine, or calcium phosphate, or via physical means such as electroporation or nucleofection.

In some embodiments, the nucleic acid is encapsulated in liposomes or lipid nanoparticles. LNPs are 100-300 nm in diameter provide efficient means of mRNA delivery to various cell types, including macrophages. In some embodiments, the nucleic acid is transferred by other nanoparticles. In some embodiments, the vector for expression of the CFP is of a viral origin, namely a lentiviral vector or an adenoviral vector. In some embodiments, the nucleic acid encoding the recombinant nucleic acid is encoded by a lentiviral vector. In some embodiments, the lentiviral vector is prepared in-house and manufactured in large scale for the purpose. In some embodiments, commercially available lentiviral vectors are utilized, as is known to one of skill in the art.

In some embodiments, the viral vector is an Adeno-Associated Virus (AAV) vector.

The methods find use in a variety of applications in which it is desired to introduce an exogenous nucleic acid into a target cell and are particularly of interest where it is desired to express a protein encoded by an expression cassette in a target cell, where the target cell or cells are part of a multicellular organism. The transposase system may be administered to the organism or host in a manner such that the targeting construct is able to enter the target cell(s), e.g., via an in vivo or ex vivo protocol. Such cells or organs are typically returned to a living body.

In some embodiments, the transgene encoding a fusion protein related to immune function is stably integrated in a living cell of a subject ex vivo, following which the cell comprising the transgene is returned to the subject. Of exemplary importance, the CFP transgene (phagocytic receptor fusion protein) is intended for expression in an immune cell, such as a myeloid cell, a phagocytic cell, a macrophage, a monocyte or a cell of dendritic cell lineage is contacted ex vivo with the recombinant nucleic acids for stable transfer of the transgene and re-introduced in the same subject for combating a disease of the subject.

The diseases contemplated comprises infectious diseases, cancer and autoimmune diseases. The nucleic acid encoding the PSR subunit comprising fusion protein (CFP) described herein is used to generate engineered phagocytic cells for treating cancer.

Cancers include, but are not limited to T cell lymphoma, cutaneous lymphoma, B cell cancer (e.g., multiple myeloma, Waldenstrom's macroglobulinemia), the heavy chain diseases (such as, for example, alpha chain disease, gamma chain disease, and mu chain disease), benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers can be characterized in various other ways including, but not limited to, serous, endometrioid, mucinous, clear cell, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In general, cellular immunotherapy comprises providing the patient a medicament comprising live cells, which should be HLA matched for compatibility with the subject, and such that the cells do not lead to graft versus Host Disease, GVHD. A subject arriving at the clinic for personalized medicine and immunotherapy as described above, is routinely HLA typed for determining the HLA antigens expressed by the subject.

Therapeutic Advantages of mRNA Driven Delivery

In one embodiment, provided herein is a method of introducing a nucleic acid sequence into a cell for sustained gene expression in the cell without adverse effects. In some embodiments, the cell is within a living system, e.g., a host organism such as a human. The nucleic acid sequence is an mRNA.

In particular, delivery via retrotransposon poses to be a highly lucrative mode. mRNA driven delivery simplifies gene delivery. While other technologies require expensive and sophisticated design and manufacturing, and a solution for delivery of the nucleic acid into the cell, and gene editing technologies to assist in integration, retrotransposon mediated delivery itself encodes for the editing machinery, encodes for new genes to be delivered. In addition, a single mRNA may be sufficient for gene delivery and editing.

In one embodiment, mRNA delivery is advantageous in that it can ensure introduction of a nucleic acid cargo without size restraint.

Table 9 summarizes some of the advantages over the other existing methods of nucleic acid deliveries.

TABLE 9
Advantages of retrotransposon mediated gene delivery

| | Lentiviral | | Retrotransposon |
|---|---|---|---|
| | delivery | AAV-delivery | delivery |
| Payload | ~4 kb | ~4 kb | >10 kb |
| Toxicity | Insertional | Unresolved | Unknown, |
| | mutagenesis | liver & CNS | pending |
| | | toxicity | clinical |
| | | | development |
| Manufacturing | Complex, | Complex, | Inexpensive, |
| | expensive | expensive | rapid |

Retrotransposons are advantageous for applications across multiple modalities. Gene manipulation using this method is easily attained both in vivo and ex vivo. In one embodiment, the application of retrotransposon may be in vivo, a piece of genetic material encoded in an mRNA can be directly introduced into a patient by systemic or local introduction. In contrast, cells can be taken out from a subject, and manipulated ex vivo and then introduced either to the same subject (autologous) or to another human (heterologous).

In one embodiment, retrotransposons and the related methods described herein may be instrumental in gene therapy. With the advantage of capacity to introduce large payloads, large sections of DNA carrying a gene encoding an entire protein may be introduced in one shot without requiring multiple introductions and multiple editing events. In one embodiment, for example, a gene that encodes a defective protein may be excised, the correct gene may be introduced in the correct site in one integration event using a retrotransposon mediated delivery. In one example, CRISPR editing may be used to excise a gene from precise locus and retrotransposition may be used to replace the correct genes. In some embodiments, a preferred retrotransposon integration site may be introduced at the excision site.

In one embodiment, retrotransposons and the related methods described herein may be instrumental in gene editing.

In one embodiment, retrotransposons and the related methods described herein may be instrumental in transcriptional regulation.

In one embodiment, retrotransposons and the related methods described herein may be instrumental in genome engineering.

In one embodiment, provided herein is a composition for incorporating a heterologous nucleic acid sequence in the genome comprising one or more polynucleic acids further comprise (i) a sequence encoding an integrase or a fragment thereof for site directed integration of the insert sequence into the genome and (ii) a genome landing site sequence that operable by the integrase, wherein the genome landing sequence is greater than 4, 5, 6, 7, 8, 9, or 10 consecutive nucleotides long. In some embodiments the integrase is a bacterial integrase. In some embodiments, the integrase is a serine integrase.

In some embodiments, the composition comprises an ORF2 and an integrase or a fragment thereof, wherein the integrase and the ORF2 are on separate polynucleotides. In some embodiments, the integrase has a capability of integrating nucleic acid sequence into a genomic site that has a genomic landing sequence that is about 10 nucleotides long, about 20 nucleotides long, about 30 nucleotides long, about 40 nucleotides long.

In some embodiments, the ORF2 and the integrase are on a single polynucleotide. In some embodiments, the ORF2 is modified to incorporate a fragment of an integrase protein that can recognize a genomic landing sequence of greater than 10 consecutive nucleotides long, and wherein the LINE retrotransposon system integrates the heterologous genomic insert into the genomic landing sequence recognized by the fragment of an integrase protein that has been incorporated into the genome.

In some embodiments, the integrase is not integrated into the genome of the cell. In some embodiments, the integrase is a recombinant protein. In some embodiments the ORF protein, e.g., the ORF2 protein is a recombinant (e.g., chimeric) protein, which comprises at least a fragment of a bacterial serine integrase that is capable of recognizing a genomic landing sequence of an integrase, e.g., a bacterial integrase, e.g., a bacterial serine integrase, wherein the genome landing sequence that is operable by the integrase and is greater than 20 nucleotides long, or greater than 30 nucleotides long. In some embodiments, the one or more of the ORF protein sequence comprise a mutation. In some embodiments, the recombinant (e.g., chimeric) ORF protein comprises a deletion of the target recognition sequence. In some embodiments, the recombinant (e.g., chimeric) ORF protein comprises a fragment of the integrase, e.g., a bacterial integrase, e.g., a bacterial serine integrase, which comprises a catalytic domain or a fragment thereof, a site-specific genomic integration recognition domain.

Provided herein is a pharmaceutical composition, wherein the insert sequence comprises an attachment site operable by the integrase.

In some embodiments, the genomic landing site is inserted into the genome using a guide RNA and a Cas system. In some embodiments, the guide RNA, the CAS system and the genomic landing sequence are in a polynucleotide that is separate from the polynucleotide comprising the sequence encoding the LINE1-ORFs and the insert sequence.

Provided herein is a method for a site-specific integration of a heterologous genomic insert sequence into the genome of a mammalian cell, the method comprising: (i) introducing into the cell (a) a polynucleotide comprising sequences encoding one or more human retrotransposon elements associated with the heterologous insert sequence, and (b) a polynucleotide comprising sequence encoding a guide RNA, an RNA guided integrase or a fragment thereof and a landing sequence operable by the integrase; (ii) verifying the integration of the heterologous insert sequence into the site of the genome.

Provided herein is a method for site-specific integration of a heterologous genomic insert using a LINE retrotransposon system, wherein the LINE retrotransposon system is modified to incorporate a fragment of an integrase protein that can recognize a genomic landing sequence of greater than 10 consecutive nucleotides long, and wherein the LINE retrotransposon system integrates the heterologous genomic insert into the genomic landing sequence recognized by the fragment of the integrase protein that has been incorporated into the genome. In some embodiments, the integrase recognizes and contacts the genomic landing sequence, and the reverse transcriptase elements of the LINE (L1) retrotransposon system reverse transcribes and incorporates the insert sequence (e.g., the cargo sequence) at the specific site of the genome landing sequence. In some embodiments, the method comprises a step of incorporating into the genome the genomic landing sequence of greater than 10 consecutive nucleotides long. In some embodiments, the step of incorporating into the genome the genomic landing sequence is performed by an RNA-guided CRISPR-Cas system. In some embodiments, the RNA-guided CRISPR-Cas system has an editing function capable of incorporating a sequence of greater than 10 consecutive nucleotides long into a specific genome site.

Therefore, provided herein is a modified L1 retrotransposition system comprising a site specific integrase DNA recognition moiety; wherein the integrase DNA recognition moiety recognizes the genomic sequence for site-specific integration, and wherein the L1 retrotransposition system reverse transcribes and integrates the sequence comprising the heterologous insert into the genomic site at or near the site recognized by the integrase. Existing systems incorporating an integrase, such as a serine integrase may comprise a DNA integration system, using for example a plasmid, a viral delivery system, each of which can be bypassed for the safer and sure mRNA system as used herein, and without the limitation of cargo size.

In one embodiment, retrotransposons and the related methods described herein may be instrumental in developing cell therapy, for example chimeric antigen receptor (CAR)T cells, in NK cell therapy or in myeloid cell therapy. In one embodiment, retrotransposons and the related methods described herein may be instrumental in delivery of genes into neurons, which are difficult to access by existing technologies.

In one aspect, provided herein is a method for targeted replacement of a genomic nucleic acid sequence of a cell, the method comprising: (A) introducing to the cell a polynucleotide sequence encoding a first protein complex comprising a targeted excision machinery for excising from the genome of the cell a nucleic acid sequence comprising one or more mutations; and (B) a recombinant mRNA encoding a second protein complex, wherein the recombinant mRNA comprises: (i) a nucleic acid sequence comprising the excised nucleic acid sequence in (A) that does not contain the one or more mutations, and (ii) a sequence encoding an L1 retrotransposon ORF2 protein under the influence of an independent promoter.

In one embodiment, the first protein complex may be an endonuclease complex independent of the second protein complex. In one embodiment, the first protein complex comprises a CRISPR-CAS system that uses sequence guided genomic DNA excision. In one embodiment, the methods described herein couples a CRISPR CAS system or any other gene editing system with a L1 transposon machinery (e.g., the second protein complex) that delivers a replacement gene with a payload capacity of greater than 4kb, or 5kb, or 6kb, or 7kb, or 8kb or 9 kb or 10 kb. This coupling can be utilized in precisely excising a large fragment (a mutated gene causing a disease) from the genomic locus and integrating a large fragment of a gene or an entire gene that encodes a correct, non-mutated sequence.

A large number of genetic diseases may require delivery of gene delivery of large payloads, often exceeding the functional capacity of existing methods. Contemplated herein are methods and compositions disclosed herein that can be instrumental in further designing therapy for such diseases using retrotransposons. An exemplary list of genetic diseases include but are not limited to the ones listed in Table 10.

TABLE 10
List of potential gene therapy applications

| Disease | Gene | CDS | | Expression | Prevalence |
|---|---|---|---|---|---|
| Stargardt | ABCA4 | 6.8 | kb | Rod and Cone | 1:8000 |
| | | | | PRs | |
| Usher 1B | MY07A | 6.7 | kb | RPE and PRs | 3.2:100,000 |
| LCA10 | CEP290 | 7.4 | kb | PR (pan | 1:50,000 |
| | | | | retinal) | |
| USH1D, | CDH23 | 10.1 | kb | PR | 3:100,000 |
| DFNB12 | | | | | |
| RP | EYS | 9.4 | kb | PR ECM | 1:50,000 |
| USH2A | USH2a | 15.6 | kb | Rod and Cone | 4:100,000 |
| | | | | PRs | |
| USH2C | GPR98 | 18.0 | kb | Mainly PRs | 1:100,000 |
| Alstrom | ALMS1 | 12.5 | kb | Rod and Cone | 1:1,000,000 |
| syndrome | | | | PRs | |
| Glycogen | GDE | 4.6 | kb | Muscle, Liver | 1:8000 |
| storage | | | | | |
| disease III | | | | | |
| Non-syndromic | OTOF | 6.0 | kb | Ear | 14:100,000 |
| deafness | | | | | |
| Hemophilia A | F8 | 7.1 | kb | Liver | 1:10,000 |
| Leber congenital | CEP290 | 7.5 | kb | Retina | 5:100,000 |
| aumaurosis | | | | | |

Provided herein is a method for targeted replacement of a genomic nucleic acid sequence in a cell. In one embodiment, the method comprises: (A) excising from the genome of the cell a nucleic acid sequence comprising one or more mutations and (B) introducing into the cell a recombinant mRNA encoding: (i) a nucleic acid sequence comprising a wild type sequence relative to the sequence excised in (A) that does not contain the one or more mutation, (ii) a sequence encoding an L1 retrotransposon ORF2 protein under the influence of an independent promoter. In one embodiment, Step (A) further comprises introducing a short sequence comprising at least a plurality of adenylate residues at the excision site. In one embodiment, the In one embodiment, the nucleic acid sequence comprising a wild type sequence is operably linked with the ORF2 encoding sequence in a way such that the ORF2 reverse transcriptase integrates the sequence comprising the wild type non-mutated sequence into the genome.

In one embodiment, the cell is a lymphocyte.

In one embodiment, the cell is an epithelial cell. In some embodiments the cell is a retinal pigmented epithelial cell (RPE).

In one embodiment, the cell is a neuron.

In one embodiment, the cell is a myeloid cell.

In one embodiment, the cell is a stem cell.

In one embodiment, the cell is a cancer cell.

In one embodiment, the gene is selected from a group consisting of ABCA4, MY07A, CEP290, CDH23, EYS, USH2a, GPR98, ALMS1, GDE, OTOF and F8.

In one embodiment, the mRNA comprises a sequence for an inducible promoter.

In one embodiment, the expression of the nucleic acid sequence comprising a non-mutated sequence is detectable at least 20, 21, 22, 23, 24, 25, 26, 27, 28, 29, 30, 31, 32, 33, 34 or 35 days post infection.

In one embodiment, the method comprises introducing into the cell a recombinant mRNA in vivo.

In one embodiment, the method comprises introducing into the cell a recombinant mRNA ex vivo.

Provided herein is a method of treating a genetic disease in a subject in need thereof, comprising: introducing into the subject a composition comprising a polycistronic mRNA encoding a gene or fragment thereof, operably linked to a sequence encoding an L1 retrotransposon; wherein the gene or the fragment thereof is at least 10.1 kb in length.

In one embodiment, the method comprises directly introducing the mRNA systemically.

In one embodiment, the method comprises directly introducing the mRNA locally.

In one embodiment, the genetic disease is a retinal disease. For example, the disease is macular dystrophy. In one embodiment, the disease is Stargardt disease, also known as juvenile macular degeneration, or fundus flavimaculatus. The disease causes progressive degeneration and damage of the macula. The condition has a genetic basis due to mutation in the ATP-binding cassette (ABC) transporter gene, (ABCA4) gene, and arises from the deposition of lipofuscin-like substance in the retinal pigmented epithelium (RPE) with secondary photoreceptor cell death. In some embodiments, the method comprises direct delivery of the mRNA to the retina.

In one embodiment, the method comprises treating a nonsyndromic autosomal recessive deafness (DFNBI12) and deafness associated with retinitis pigmentosa and vestibular dysfunction (USHID). In one embodiment, provided herein is a method of treating non-syndromic deafness (DFNB12) or Usher syndrome (USH1D), the method comprises introducing an mRNA comprising a copy of CDH23 or a fragment thereof operably linked to a sequence encoding an L1 retrotransposon.

### Cell Specific Expression of Exogenous Polypeptide

Stable expression of an exogenous polypeptide may be accomplished in a variety of cell types (e.g. target cell types) using a mobile genetic element to target integration of a polynucleotide sequence (e.g. often referred to herein as an insert sequence in a construct) in the genome of the cell (e.g. a target cell). In some embodiments, the target cell is a post-mitotic cell, e.g., a mammalian cardiomyocyte, or an RPE cell. In some embodiments, the mobile genetic element comprises a human LINE 1 sequence. In some embodiments, the mobile genetic element is a human LINE1 sequence. In some embodiments, the mobile genetic element comprises a sequence encoding a human L1 ORFp1 protein. In some embodiments, the mobile genetic element comprises a sequence encoding a human L1-ORFp2 protein. In some embodiments, the mobile genetic element comprises a sequence encoding a human ORFp1 and human ORFp2 polypeptides. In some embodiments, a polynucleotide sequence is introduced in a target cell, the polynucleotide comprises of a sequence encoding a mobile genetic element, and an insert sequence. In some embodiments, the mobile genetic element comprises a polypeptide that post-translationally promotes a stable integration of an insert sequence into the genome of the target cell. In some embodiments, the mobile genetic element comprises a polypeptide further configured for site-specific integration of a given insert sequence into the genome of the target cell, for example but not limited to the embodiments as is described elsewhere in the specification.

In some embodiments, the polynucleotide described herein is an mRNA. The mRNA may be bicistronic or polycistronic. As is exemplified by the working embodiments, the insert sequence and the sequence encoding the mobile genetic element can be in reverse orientation with respect to each other. The mobile genetic element may integrate the insert sequence via target-primed reverse transcription (TPRT).

In some embodiments, the mobile genetic element comprises the human L1 retrotransposon, or fragments thereof.

In some embodiments, the polynucleotide can be specifically targeted to a cell type. In some embodiments, the polynucleotide may be composed in a nanoparticle, wherein the nanoparticle comprises one or more targeting moieties known to one of skill in the art.

Provided herein is a method of stably integrating an insert sequence into genomic DNA of a target cell, the method comprising contacting a composition to the target cell, the composition comprising a polynucleic acid, wherein the polynucleic acid comprises: an insert sequence, wherein the insert sequence comprises a sequence that is a reverse complement of a sequence encoding an exogenous polypeptide, and a mobile genetic element comprising a sequence encoding a polypeptide, wherein the polypeptide encoded by the sequence of the mobile genetic element promotes integration of the insert sequence into genomic DNA; stably integrating the insert sequence into the genomic DNA of the target cell; and expressing an exogenous polypeptide in the target cell, wherein the target cell is a human hepatocyte. In some embodiments polynucleic acid is mRNA. In some embodiments, the mRNA is encapsulated in a nanoparticle for intracellular delivery. In some embodiments, the nanoparticle comprises a lipid. In some embodiments, the polynucleic acid is introduced into the hepatocyte by contacting a composition, comprising, for example, the polynucleic acid and a delivery vehicle (e.g., a nanoparticle comprises a lipid) to the hepatocyte such that it results in the successful uptake of the polynucleic acid by the hepatocyte. In some embodiments, the incorporation is via electroporation. In some embodiments, the polynucleic acid composition is electroporated in the hepatocyte. In some embodiments, the hepatocyte is electroporated under conditions suitable for expression of the polynucleic acid-encoded polypeptides and conducive to the viability of the hepatocyte. In some embodiments the integration of the insert into the genome of the hepatocyte is verified after incorporation the polynucleic acid by methods known to one of skill in the art, e.g., by genome sequencing. In some embodiments, the expression of the polypeptide from integrated insert sequence is verified at a suitable interval following incorporation of the polynucleic acid in the hepatocyte, wherein the suitable interval is about 4, 6, 8, 10, 12, 24 or 48 hours. In some embodiments, following electroporation of a population of cells comprising hepatocytes, the population of cells comprising the hepatocytes that have been subjected to the electroporation is cultured under conditions suitable for a hepatocyte for at least about 2 hours, about 3 hours, about 4 hours, about 5 hours, about 6 hours, about 8 hours, about 10 hours or about 24 hours. In some embodiments, the hepatocyte is cultured for about 48 hours, 72 hours, 96 hours or more under conditions suitable for a hepatocyte for growth. In some embodiments, the expression of a polypeptide encoded by the polynucleotide (e.g., the insert) is verified after 48, 72, or 96 hours, or after 4 days, 5 days, 6 days, 7 days, 8 days, 9 days, 10 days or more in culture.

In some embodiments, at least 2% of the human hepatocytes express the exogenous polypeptide at day 10 after incorporating. In some embodiments, about 2%, or about 5%, or about 10% or more of the human hepatocytes in the population of cells subjected to the electroporation express the exogenous polypeptide at day 10 after incorporating.

Provided herein is a method of stably integrating an insert sequence into genomic DNA of a target cell, the method comprising contacting a composition to the target cell, the composition comprising a polynucleic acid, wherein the polynucleic acid comprises: an insert sequence, wherein the insert sequence comprises a sequence that is a reverse complement of a sequence encoding an exogenous polypeptide, and a mobile genetic element comprising a sequence encoding a polypeptide, wherein the polypeptide encoded by the sequence of the mobile genetic element promotes integration of the insert sequence into genomic DNA; stably integrating the insert sequence into the genomic DNA of the target cell; and expressing an exogenous polypeptide in the target cell, wherein the target cell is a human cardiomyocyte. In some embodiments polynucleic acid is mRNA. In some embodiments, the mRNA is encapsulated in a nanoparticle for intracellular delivery. In some embodiments, the nanoparticle comprises a lipid. In some embodiments, the polynucleic acid is introduced into the cardiomyocyte by contacting a composition, comprising, for example, the polynucleic acid and a delivery vehicle (e.g., a nanoparticle comprises a lipid) to the cardiomyocyte such that it results in the successful uptake of the polynucleic acid by the cardiomyocyte. In some embodiments, the incorporation is via electroporation. In some embodiments, the polynucleic acid composition is electroporated in the cardiomyocyte. In some embodiments, the cardiomyocyte is electroporated under conditions suitable for expression of the polynucleic acid-encoded polypeptides and conducive to the viability of the cardiomyocyte. In some embodiments the integration of the insert into the genome of the cardiomyocyte is verified after incorporation the polynucleic acid by methods known to one of skill in the art, e.g., by genome sequencing. In some embodiments, the expression of the polypeptide from integrated insert sequence is verified at a suitable interval following incorporation of the polynucleic acid in the cardiomyocyte, wherein the suitable interval is about 4, 6, 8, 10, 12, 24 or 48 hours. In some embodiments, following electroporation of a population of cells comprising cardiomyocytes, the population of cells comprising the cardiomyocytes that have been subjected to the electroporation is cultured under conditions suitable for a cardiomyocyte for at least about 2 hours, about 3 hours, about 4 hours, about 5 hours, about 6 hours, about 8 hours, about 10 hours or about 24 hours. In some embodiments, the cardiomyocyte is cultured for about 48 hours, 72 hours, 96 hours or more under conditions suitable for a cardiomyocyte for growth. In some embodiments, the expression of a polypeptide encoded by the polynucleotide (e.g., the insert) is verified after 48, 72, or 96 hours, or after 4 days, 5 days, 6 days, 7 days, 8 days, 9 days, 10 days or more in culture. In some embodiments, at least 2% of the human cardiomyocytes express the exogenous polypeptide at day 10 after incorporating. In some embodiments, about 2%, or about 5%, or about 10% or more of the human cardiomyocytes in the population of cells subjected to the electroporation express the exogenous polypeptide at day 10 after incorporating. Provided herein is a method of stably integrating an insert sequence into genomic DNA of a target cell, the method comprising contacting a composition to the target cell, the composition comprising a polynucleic acid, wherein the polynucleic acid comprises: an insert sequence, wherein the insert sequence comprises a sequence that is a reverse complement of a sequence encoding an exogenous polypeptide, and a mobile genetic element comprising a sequence encoding a polypeptide, wherein the polypeptide encoded by the sequence of the mobile genetic element promotes integration of the insert sequence into genomic DNA; stably integrating the insert sequence into the genomic DNA of the target cell; and expressing an exogenous polypeptide in the target cell, wherein the target cell is a human retinal pigment epithelial cell (RPE). In some embodiments polynucleic acid is mRNA. In some embodiments, the mRNA is encapsulated in a nanoparticle for intracellular delivery. In some embodiments, the nanoparticle comprises a lipid. In some embodiments, the polynucleic acid is introduced into the RPE by contacting a composition, comprising, for example, the polynucleic acid and a delivery vehicle (e.g., a nanoparticle comprises a lipid) to the RPE such that it results in the successful uptake of the polynucleic acid by the RPE. In some embodiments, the incorporation is via electroporation. In some embodiments, the polynucleic acid composition is electroporated in the RPE. In some embodiments, the RPE is electroporated under conditions suitable for expression of the polynucleic acid-encoded polypeptides and conducive to the viability of the RPE. In some embodiments the integration of the insert into the genome of the RPE is verified after incorporation the polynucleic acid by methods known to one of skill in the art, e.g., by genome sequencing. In some embodiments, the expression of the polypeptide from integrated insert sequence is verified at a suitable interval following incorporation of the polynucleic acid in the RPE, wherein the suitable interval is about 4, 6, 8, 10, 12, 24 or 48 hours. In some embodiments, following electroporation of a population of cells comprising RPEs, the population of cells comprising the RPEs that have been subjected to the electroporation is cultured under conditions suitable for a RPE for at least about 2 hours, about 3 hours, about 4 hours, about 5 hours, about 6 hours, about 8 hours, about 10 hours or about 24 hours. In some embodiments, the RPE is cultured for about 48 hours, 72 hours, 96 hours or more under conditions suitable for a RPE for growth. In some embodiments, the expression of a polypeptide encoded by the polynucleotide (e.g., the insert) is verified after 48, 72, or 96 hours, or after 4 days, 5 days, 6 days, 7 days, 8 days, 9 days, 10 days or more in culture. In some embodiments, at least 2% of the human RPEs express the exogenous polypeptide at day 10 after incorporating. In some embodiments, about 2%, or about 5%, or about 10% or more of the human RPEs in the population of cells subjected to the electroporation express the exogenous polypeptide at day 10 after incorporating.

Provided herein is a method of stably integrating an insert sequence into genomic DNA of a target cell, the method comprising contacting a composition to the target cell, the composition comprising a polynucleic acid, wherein the polynucleic acid comprises: an insert sequence, wherein the insert sequence comprises a sequence that is a reverse complement of a sequence encoding an exogenous polypeptide, and a mobile genetic element comprising a sequence encoding a polypeptide, wherein the polypeptide encoded by the sequence of the mobile genetic element promotes integration of the insert sequence into genomic DNA; stably integrating the insert sequence into the genomic DNA of the target cell; and expressing an exogenous polypeptide in the target cell, wherein the target cell is a human neuronal cell. In some embodiments polynucleic acid is mRNA. In some embodiments, the mRNA is encapsulated in a nanoparticle for intracellular delivery. In some embodiments, the nanoparticle comprises a lipid. In some embodiments, the polynucleic acid is introduced into the neuronal cell by contacting a composition, comprising, for example, the polynucleic acid and a delivery vehicle (e.g., a nanoparticle comprises a lipid) to the neuronal cell such that it results in the successful uptake of the polynucleic acid by the neuronal cell.

In some embodiments, the incorporation is via electroporation. In some embodiments, the polynucleic acid composition is electroporated in the neuronal cell. In some embodiments, the neuronal cell is electroporated under conditions suitable for expression of the polynucleic acid-encoded polypeptides and conducive to the viability of the neuronal cell. In some embodiments the integration of the insert into the genome of the neuronal cell is verified after incorporation the polynucleic acid by methods known to one of skill in the art, e.g., by genome sequencing. In some embodiments, the expression of the polypeptide from integrated insert sequence is verified at a suitable interval following incorporation of the polynucleic acid in the neuronal cell, wherein the suitable interval is about 4, 6, 8, 10, 12, 24 or 48 hours. In some embodiments, following electroporation of a population of cells comprising neuronal cells, the population of cells comprising the neuronal cells that have been subjected to the electroporation is cultured under conditions suitable for a neuronal cell for at least about 2 hours, about 3 hours, about 4 hours, about 5 hours, about 6 hours, about 8 hours, about 10 hours or about 24 hours. In some embodiments, the neuronal cell is cultured for about 48 hours, 72 hours, 96 hours or more under conditions suitable for a neuronal cell for growth. In some embodiments, the expression of a polypeptide encoded by the polynucleotide (e.g., the insert) is verified after 48, 72, or 96 hours, or after 4 days, 5 days, 6 days, 7 days, 8 days, 9 days, 10 days or more in culture. In some embodiments, at least 2% of the human neuronal cells express the exogenous polypeptide at day 10 after incorporating. In some embodiments, about 2%, or about 5%, or about 10% or more of the human neuronal cells in the population of cells subjected to the electroporation express the exogenous polypeptide at day 10 after incorporating.

### EXAMPLES

It should be appreciated that the invention should not be construed to be limited to the examples which are now described; rather, the invention should be construed to include any and all applications provided herein and all equivalent variations within the skill of the ordinary artisan.

### Example 1. Exemplary Retrotransposon Designs Constructs

Provided here are exemplary strategies of designing retrotransposon constructs for incorporating into the genome of a cell and expressing an exemplary transgene. FIG. 1B and FIG. 1C illustrates various strategic designs for integrating an mRNA encoding transgene into the genome of a cell. GFP shown here in a box is an exemplary transgene. The mRNA encoding the transgene (e.g., GFP) can be co-expressed with a nucleic acid sequence encoding an ORF2p protein, in either sense or antisense orientation; the respective coding sequences may be in a monocistronic or bicistronic construct shown under exemplary Cis-strategies (FIG. 1B and FIG. 1C). CMV/T7 are promoters.

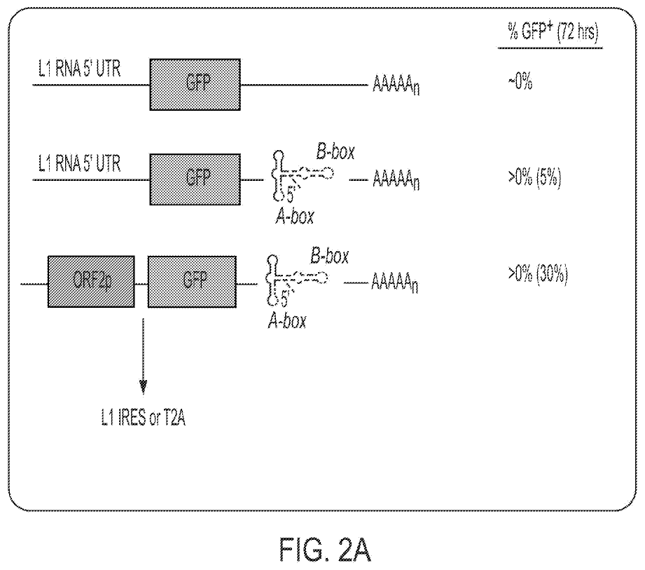

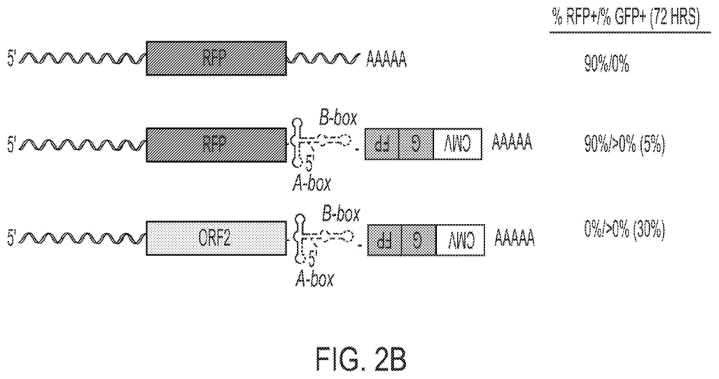

On the other hand, the same could be directed to be expressed in a trans manner. The trans-strategy can include a sequence encoding an ORF2p protein or both ORF1p and ORF2p proteins from a bicistronic sequence and an mRNA encoding a GFP in a sense or antisense direction in the 3′UTR of any gene. The transgene is flanked by a retrotransposing sequence comprising transposase binding sequences, an A-box and B-box, and a poly A tail. FIG. 2A illustrates three exemplary designs for expressing an exemplary transgene GFP by stably incorporating the sequence encoding GFP using the constructs. The first construct comprises a sequence encoding GFP, flanked by L1 5′-UTR; and a poly A sequence at the 3′ UTR, in absence of any transposase binding elements. The second and the third constructs comprise a sequence encoding GFP, a 3′UTR an A Box and a B-box, and a poly A sequence at the 3′ UTR. The third construct comprises an additional sequence encoding ORF2p. Expected GFP expression levels at 72 hours are shown on the right side. FIG. 2B illustrates three exemplary designs for expressing an exemplary transgene GFP in an mRNA that either encodes RFP or ORF2p by stably incorporating the sequence encoding GFP using the constructs. The first construct comprises a sequence encoding RFP, and a poly A sequence at the 3′ UTR, in absence of any L1 elements. The second and the third constructs comprise a 3′UTR comprising an A Box and a B-box, and a poly A sequence at the 3′ UTR. The second construct comprises a sequence encoding RFP and the third construct comprises a sequence encoding ORF2p. Expected RFP and GFP expression levels at 72 hours are shown on the right side.

### Example 2. Exemplary circRNA Designs Constructs

In this example, modular designs for circRNA are demonstrated, which incorporate a stretch of about 50 nucleotide long RNA having naturally occurring tertiary structures in order to prepare a circRNA. Use of the tertiary-structure forming RNA makes the circRNA formation process independent of sequence mediated hybridization for circularization. These RNA motifs having tertiary structures can be incorporated in the desired RNA having an exon and an intron in place of the 5′ and 3′ homology arms, thereby forming the terminal RNA scaffolds for circularization.

TectoRNA: RNA-RNA binding interfaces are constructed by combining pairs of GNRA loop/loop-receptor interaction motifs, yielding high affinity, high specificity tertiary structures. (FIG. 3B). Pairs of GNRA loop/loop-receptor interaction motifs are fused using the four-way junction from the hairpin ribozyme to create divalent, self-assembling scaffolding units (‘tectoRNA’) which help form a closed cooperatively assembling ring-shaped complexes. Using two orthogonal loop/loop-receptor interaction motifs, RNA monomers are designed that are capable of directional assembly in either the parallel (‘up-up’) or anti-parallel (‘up-down’) assembly modes. In anti-parallel assembly of interacting molecules, each incorporated monomer switches the directionality of the growing chain and thus compensates for its intrinsic bending, producing long, relatively straight multi-unit chains. For selecting a tectoRNA scaffolds having minimum occurrences of alternative secondary structures, sequences are checked by submitting them to the RNA folding program Mfold (bioinfo.math.rpi.edu/˜zukerm/rna/mfold) which predicts the thermodynamically favored secondary structure of a given RNA sequence. A thermodynamically favored structure is selected for scaffolding that has minimum alternative secondary structures (typically but not exclusively, no other secondary structure is closer than 15% in energy to the lowest energy structure). RNA molecule is prepared by conventional methods, such as in vitro run-off transcription using T7 RNA polymerase. FIG. 3B shows a RL-GAAA loop structure. In order to profile tectoRNA heterodimers a fluorescence-based chip-flow piece testing method is utilized. In this method, a library of potential variants of the structured RNA (chip piece) is synthesized as DNA templates and amplified to include sequencing adapters and regions for RNAP initiation. Each DNA variant is transcribed in situ into RNA, enabling display of sequence-identified clusters of RNA on the surface of the sequencing chip. The fluorescently-labeled tectoRNA binding partner, the “flow piece”, is introduced to the sequencing chip flow cell at increasing concentrations, allowing quantification of bound fluorescence to each cluster of RNA after equilibration. These fluorescence values are used to derive the affinity of the flow piece to each chip piece variant (FIG. 3C), in terms of the dissociation constant (K_{d}) and binding free energy, (ΔG=RT log(K_{d})).

The selected terminal RNA scaffold segments comprising the tertiary structures are incorporated using T7 transcription or ligated at the 5′ and 3′ ends of the desired RNA to be circularized; or are incorporated in the desired RNA by any known molecular biology techniques.

### Example 3: Exemplary Retrotransposon Designs with Enhanced Specificity

In this example, designs for a nucleic acid construct for L 1-mediated retrotransposon for enhanced target specificity is demonstrated. An mRNA is designed comprising ORF2 encoding sequence and a sequence encoding a gene of interest, to incorporate the gene of interest into the genome of a cell using ORF2. In one exemplary design, the construct comprises an ORF2 that is further modified.

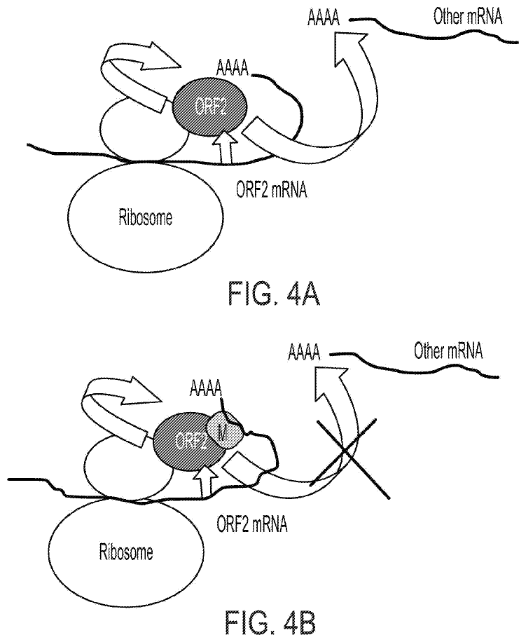

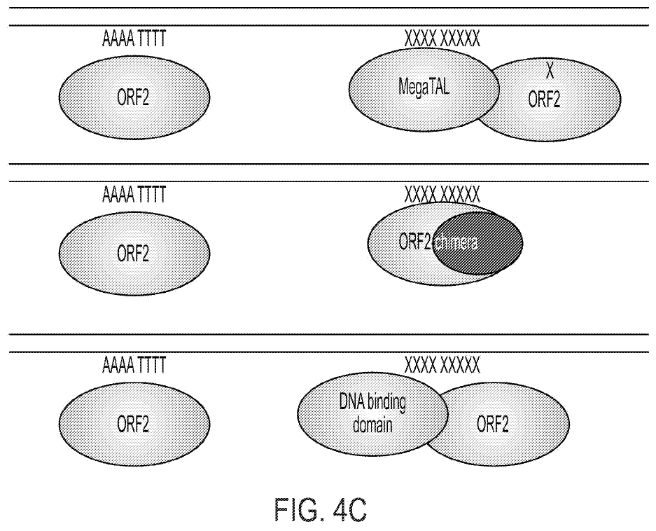

As shown in FIG. 4A, ORF2 protein initiates retrotransposition by binding to its own poly A sequence. However, because poly A is abundantly present in mRNAs, a non-specific binding and integration becomes a possibility. To increase the specificity, a recombinant ORF2 is designed comprising an mRNA-binding domain of a heterologous protein, and the cognate mRNA sequence for the heterologous mRNA-binding domain is inserted near the poly A sequence in the 3′-UTR and the ORF2 poly A binding site.

A chimeric ORF2 is thereby generated as shown in (FIG. 4B), in which a high affinity RNA-binding domain of a heterologous protein encoding sequence is incorporated or fused to the ORF2 sequence and cognate RNA sequences corresponding to the high affinity RNA-binding protein is incorporated in the 3′UTR region of the mRNA, proximal to the poly A region. In this example the heterologous high affinity RNA-binding domain is derived from MCP coat protein MS2 (shown as M in the figure), is incorporated within the ORF2 sequence and the cognate sequence, the MS2 hairpin, is included in the 3′ UTR sequence of the mRNA (FIG. 4B). The MS2 binds to the cognate sequence, increasing the specificity of the chimeric ORF2 to its own mRNA for reverse transcribing and incorporating the respective sequence associated with the ORF2 mRNA in the mammalian cell genome (FIG. 4B).

In other exemplary designs, attempts to increase specificity of integration of the transgene by the ORF2 within the genome of a target cell is undertaken. In one exemplary design, Mega TAL encoding sequence fused to an ORF2 as shown in FIG. 4C (upper panel). Along with that, the ORF2 is mutated to remove its ability to recognize and bind to RNA sequence that has less specificity. The fused protein is directed to the TAL binding sequence incorporated within the 3′UTR and perform endonuclease function.

The Mega TAL DNA binding sequence is targeted by the fusion protein. Likewise, other chimera (FIG. 4C (middle panel)) and fusion protein with a specific DNA binding domain FIG. 4C (lower panel) are designed.

### Example 4. Exemplary Plasmid Design and Developments for LINE-1 Mediated Retrotransposition of an Exogenous Nucleic Acid Sequence

In this example plasmid vectors are generated for delivery and incorporation of a recombinant LINE-1 construct comprising an ORF2 transposon element operably linked to a transgene transposable into a mammalian cell, and regulatory elements for mRNA transcription and stabilization. The mRNA can be transcribed in a bacterial host cell, which can be further processed and/or purified for introduction into a mammalian cell in vitro or administration in an organism, such as a mammal, a rodent, sheep, pig or a human.

Any suitable vector backbone is used for incorporating the recombinant nucleic acid sequence as insert and transcribing in a bacterial system for mRNA generation; or in vitro transcription system may be utilized to generate an mRNA comprising the recombinant nucleic acid sequence. Several features are added to the plasmid. Upon successful scalable mRNA production, and purification, the mRNA may be introduced in a mammalian cell of interest, such as a myeloid cell.

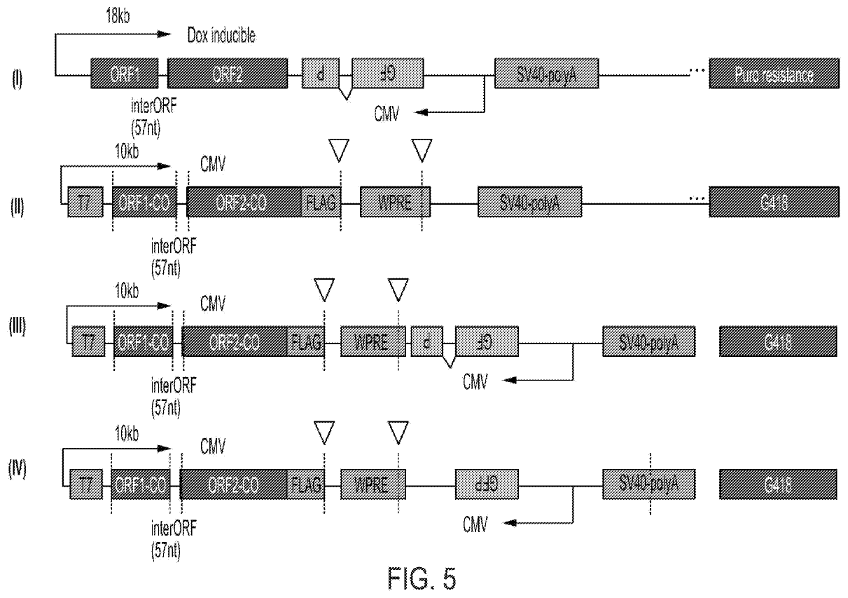

Plasmids traditionally used in the field of study for retrotransposition lack designer genes, gene blocks, and Gibson assembly methods were used regularly to insert different features. A new vector that takes features from the old vectors but has flexibility to insert new features can be beneficial both for the study and optimization of LINE-1 elements as a gene delivery system. Below is an outline of base features and additional features that can increase retrotransposition frequency, both using the plasmid alone or the mRNA transcribed from the plasmid. In an exemplary plasmid design shown graphically in FIG. 5(I), which contains the natural LINE-1 sequence with the original 5′UTR, 3′UTR and interORF sequence with no restriction sites to swap out any of these features. New optimized plasmid:
Removed Dox inducible promoter, replaced with CMV or EIF1a or EF1a promoter
Added a T7 site to make mRNA
Codon optimized ORF1 and ORF2
Added a WPRE element to stabilize mRNA
Added FLAG tag to ORF2 to help with protein detection
Decreased size from 18kb to 14kb
Added blunt restriction sites (dotted lines with blunt arrows) at each feature to facilitate insertions
Includes a G418 selection marker

The plasmid is shown in FIG. 5 (II).

With Gibson a reverse split GFP is inserted for plasmid reporter gene as shown in FIG. 5 (III). A complete reverse GFP for the mRNA reporter is inserted as in FIG. 5 (IV).

Using the plasmid construct in FIG. 5(V) as parent, a nuclear localization sequence (NLS) is inserted at the N terminus of ORF2 to help with nuclear import (FIG. 5 (VI)). An IRES or another termination/promoter sequence is inserted to increase expression of ORF2 (FIG. 5 (VII)). To facilitate stronger interactions between ORF2 and the mRNA, MS2 hairpins are inserted in the 3′UTR and a MS2 coat protein sequence in the N terminus of the ORF2 protein (FIG. 5 (VIII)). A corresponding exemplary ORF2 with enhanced specificity and its mechanism of action is disclosed in the preceding example and in FIG. 4B. To facilitate stronger interactions of the mRNA with the translating ribosome and to stall translation so that nascent ORF2 will more likely bind the mRNA, an Alu element is inserted in the 3′UTR of the mRNA (FIG. 5 (IX)). To potentially use a more active ORF2 protein, the ORF2's RT domain is replaced with the Group II intron's reverse transcriptase domain (FIG. 5(X)). Additionally, the minke whale genome has the highest number and percentage of active LINE elements (˜5,000 with 60% active compared to humans that have 480 with 3.6% active). The two sequences are 67% identical and the whale sequence has the active endonuclease and reverse-transcriptase residues. The respective minke whale domains can be used to replace native ORF2 endonuclease and/or RT domains or design a chimera domain.

### Example 5. mRNA Design Synthetic mRNA Generation

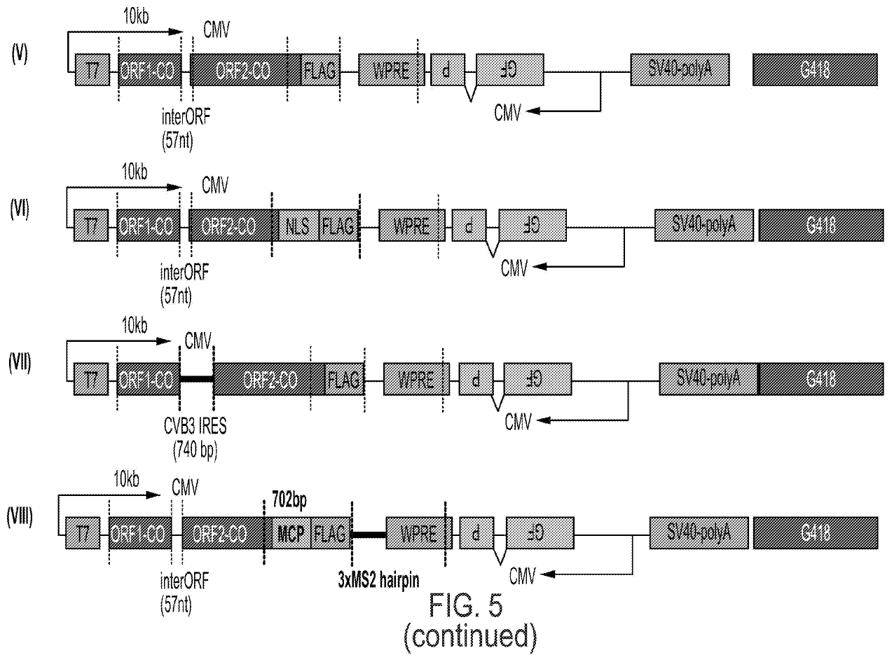

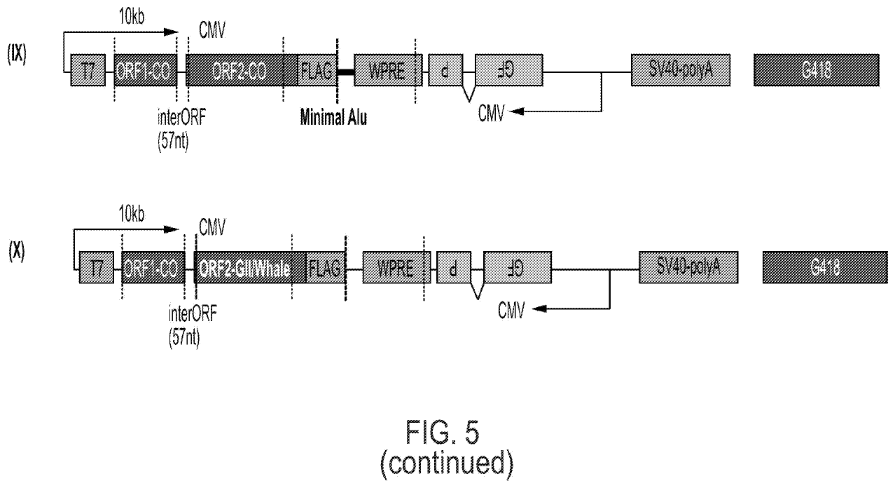

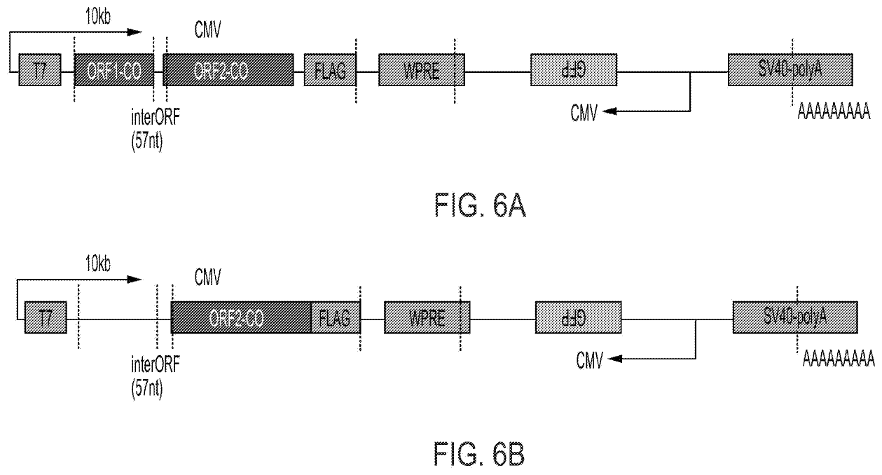

mRNA can be strategically designed for synthetic production by oligosynthesis and or ligation of oligonucleotides. Additionally, such designs are useful for in vitro transcription (IVT) mediated mRNA generation. The mRNA strategy can include the same variants as the plasmid strategy discussed in the previous example. The main differences are that the reporter GFP sequence does not include an intron (FIG. 6A) and that the constructs can be delivered without the ORF1 coding region (FIG. 6B).

### Example 6. Structural features for increased mRNA half-life

In this example, structural features are introduced in the mRNA comprising the retrotransposition elements and/or the transgene for increasing the mRNA half-life. The goal is to increase the duration of protein expression from the mRNA in primary monocytes from three days to at least 5 days with an ultimate goal of 10 days.

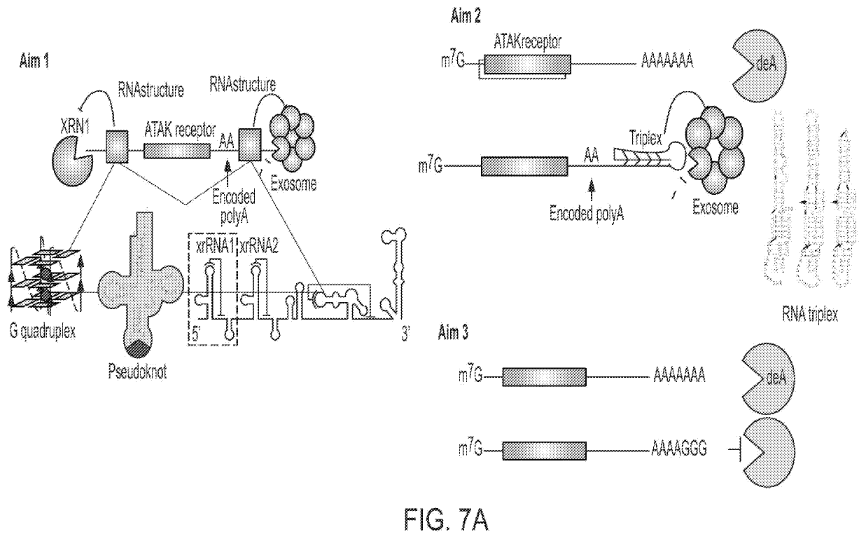

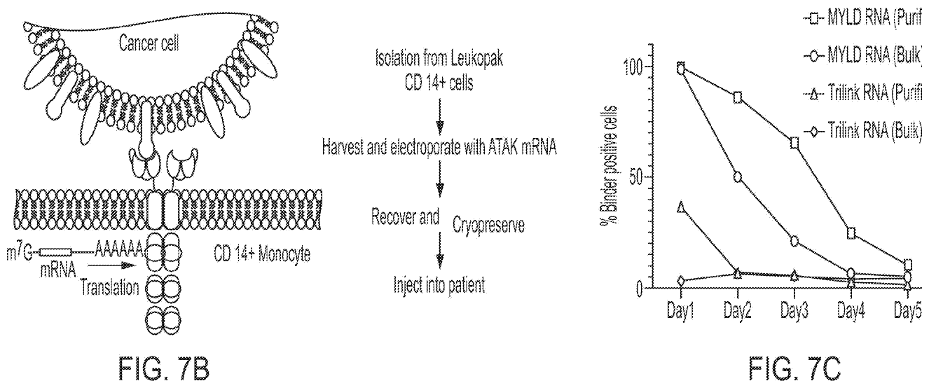

As shown in FIG. 7B (left), the mRNA comprising a sequence encoding the transgene when introduced into a CD14+ myeloid cell (monocyte), is translated and expresses a chimeric receptor (an ATAK construct) capable of binding to an antigen on a cancer cell.

A number of mRNA designs are generated by synthesizing various gene blocks comprising singly, or combinations of one or more of: (i) a G-quadruplex, (ii) a viral pseudoknot structure in the 5′ UTR; and/or (iii) one or (iv) more xrRNA loop structures in the 3′ UTR (v) a triplex RNA structure as shown in FIG. 7A; and cloned into the transcription vector at the respective UTRs adjoining the coding sequence of the transgene. These constructs are individually prepared by an off-site vendor and tested in-house for determining stability of the mRNA, as measured by the expression of the chimeric receptor (An exemplary receptor and its function is depicted graphically in FIG. 7B (left). The process flow chart is shown on FIG. 7B (right). In short, constructs are cloned into plasmids, with encoded or modified poly A tails. The mRNA was transcribed and purified. Meanwhile, frozen monocytes are thawed and harvested.

Harvested cells were electroporated with the purified mRNA (5-10 ug), and cultured for 1, 2, 3, 5 days. Cells positive for the chimeric receptor (binder positive cells), are detected by means of their ability to bind to a target cell or a substrate coated with the target antigen. The expected results are shown in FIG. 7C.

Bulk or purified mRNA expressing one or more of the structural features outlined in (i)-(v) (data denoted by solid squares) or a combination thereof outperforms the commercially available counterparts that do not contain any of the features outlined in (i)-(v) (data denoted by triangles).

### Example 7. LINE-1 Retrotransposon Plasmid Mediated Delivery of GFP Gene

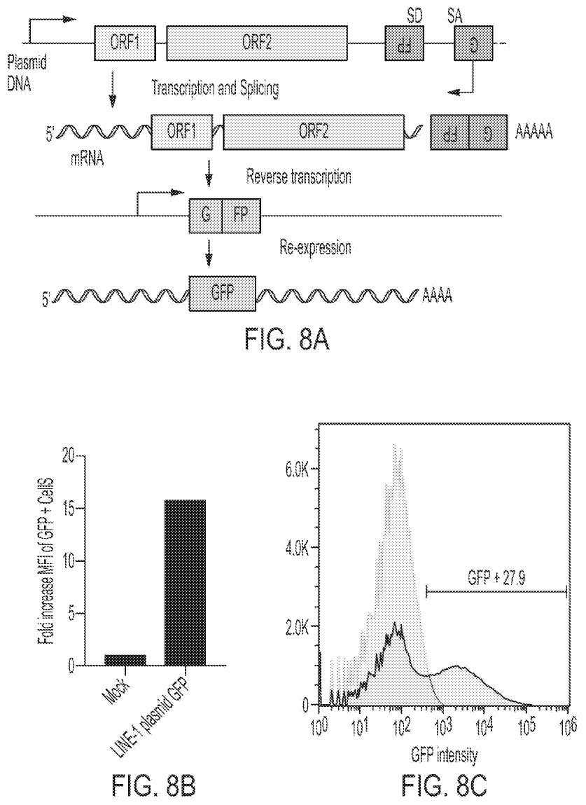

In this test run, genomic integration of a GFP cargo and expression the GFP protein using a LINE-1 retrotransposon system was verified. The LINE-1-GFP construct (LINE-1 plasmid GFP) is exemplified in FIG. 8A: A plasmid construct having a LINE-1 sequence encoding ORF1p (ORF1), a sequence encoding ORF2p (ORF2), and a CMV promoter driven split GFP gene situated in the 3′UTR of the LINE-1 in reverse orientation with respect to the ORFs. The split GFP is designed to have an intronic sequence inserted in between a splice donor and acceptor sites, which ensures that the GFP is expressed only after integration and splicing mediated removal of the noncoding sequence in the middle of the coding sequence. In this case the cargo is 2.1 kb. HEK293T cells were transfected with the plasmid using Fugene reagent, and plasmid positive cells were selected by puromycin. The mRNA generated from a genome integrated GFP successfully translates and is measured by flow cytometry, as indicated as change in mean fluorescence intensity (MFI) (FIG. 8B) and fraction of cells with GFP fluorescence intensity compared to mock transfected cells (FIG. 8C). Mock transfected cells received the plasmid that lack the GFP sequence.

### Example 8. LINE-1 Retrotransposon Plasmid Mediated Delivery of a Chimeric Receptor Gene

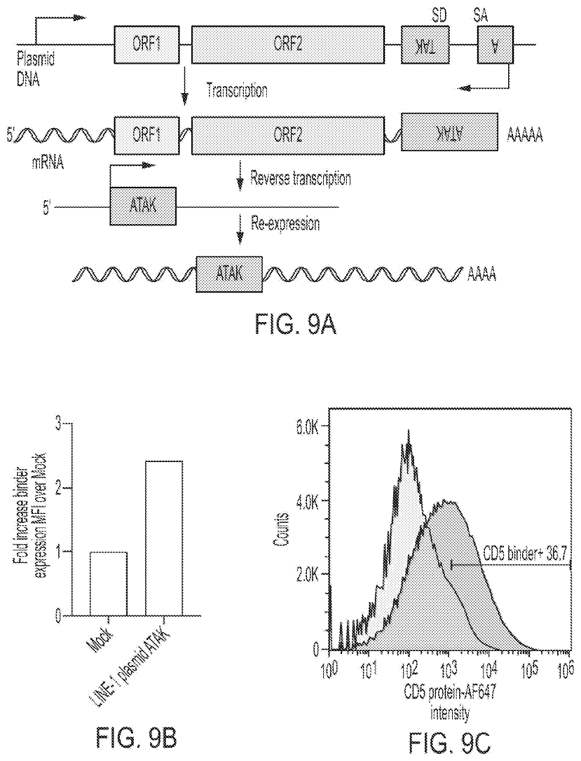

This example demonstrates that a recombinant gene can be successfully expressed using the LINE-1 sequence in a cell. HEK 293 cells were transfected with a plasmid having the LINE-1 elements, with a 3 kb cargo sequence encoding recombinant receptor protein CD5-intron-fcr-PI3K (ATAK) that is interrupted by an intron sequence in the CD5 binding domain. The cargo is a chimeric receptor that has a CD5 binding extracellular domain, a FCRγ transmembrane domain, and an intracellular domain having a P13-kinase recruitment domain. The schematic representation of the retrotransposon plasmid is shown in FIG. 9A. As in the design of the experiment above, the ATAK receptor cannot express unless it is integrated in the genome and the intron is spliced off. Following transfection in HEK293T cells, the receptor expression is detected using labeled CD5 as bait for the CD5 binding extracellular domain. Results shown in FIGS. 9B and 9C show successful integration and expression of the receptor. 36.5% cells were ATAK (CD5 binder) positive (FIG. 9C).

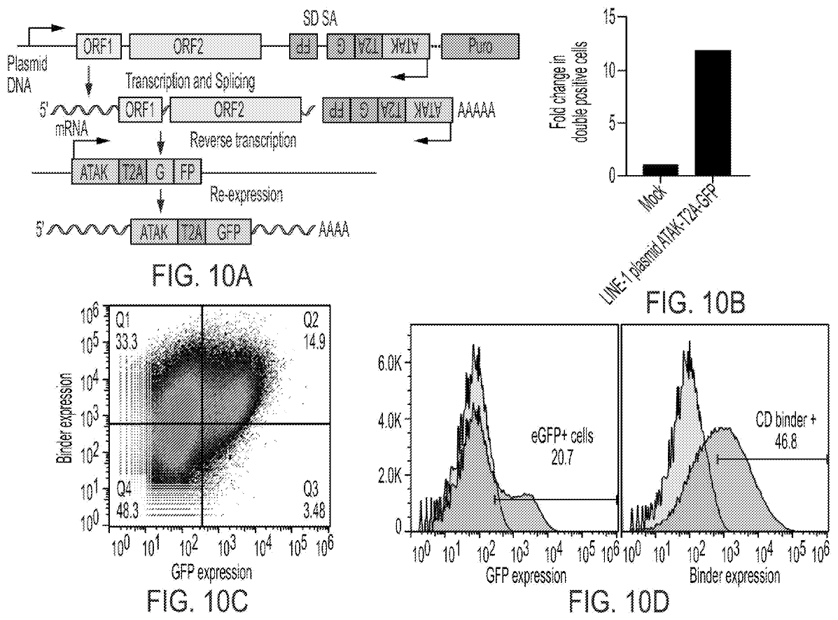

In a further modification, a LINE-1 construct (LINE-Iplasmid-cd5 fcr-pi3k_t2a_GFPintron) with a longer 3.7 kb cargo sequence encoding a non-interrupted recombinant receptor protein CD5-intron-fcr-PI3K and an interrupted GFP sequence with a T2A sequence between receptor and the GFP sequences (FIG. 10A). Normalized against mock-transfected cells, there was a greater than 10-fold increase of the ATAK receptor and GFP double-positive cells was noted (FIG. 10B). Exemplary fluorescence identification of GFP and fluorescent tagged CD5 binding and gating quantitation for experimental runs are shown in FIG. 10C and FIG. 10D.

### Example 9. mRNA Encoding LINE-1 Retrotransposon for Delivery of a Cargo Gene

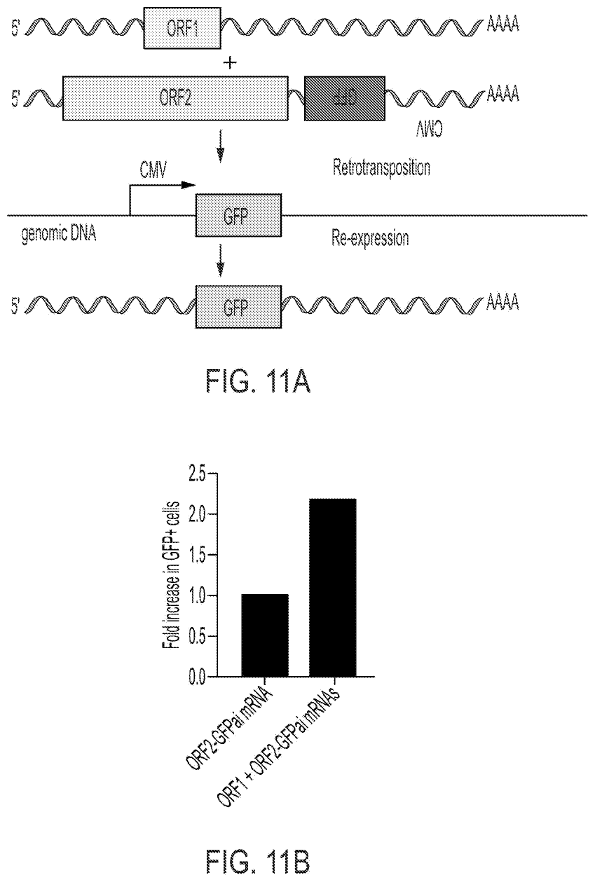

In this assay, capability of delivering and expressing a LINE-1 retrotransposable gene sequence as an mRNA was tested. An mRNA encoding an ORF1 (ORF1-FLAG-mRNA), and an mRNA encoding ORF2 and GFP in the antisense direction with a CMV promoter sequence (ORF2-FLAG-GFPai) are designed as shown in FIG. 11A. The cargo size in this assay was 2.4 kb, and GFP is in antisense orientation with respect to ORF2 sequence. The mRNAs were electroporated in 293T cells and the reporter genes expression was demonstrated as shown in FIG. 11B. This experimental set up demonstrated that no ORF1-readthrough is necessary for the expression of the ORF2p, and expression of ORF2p from a different mRNA molecule can allow higher expression of ORF2p and GFP. With these results, a successful delivery of the LINE-1 and cargo in the form of mRNA was achieved.

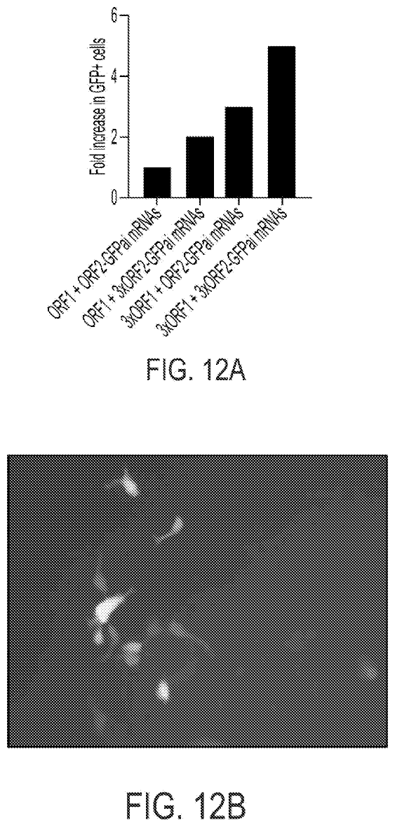

In order to determine whether the relative levels of ORF1 and ORF2 mRNA affected GFP expression an experiment was set up to test the varying amounts of ORF1 and ORF2 mRNAs (FIG. 11A). 3× the amount of each and together is tested for increases in GFP+ cells and results are shown in FIG. 12A. Fold increase is relative to 1× ORF2-GFP and 1× ORF1 mRNA. GFP expression was higher when 3× ORF1 was used with 1× ORF2, but not the reverse; whereas having both 3× ORF1 and 3× ORF2 showed the maximum level of GFP expression in the sets compared. The cargo size here is 2.4 kb. FIG. 12B shows fluorescent microscopy image of GFP+ cells following retrotransposon mRNA electroporation.

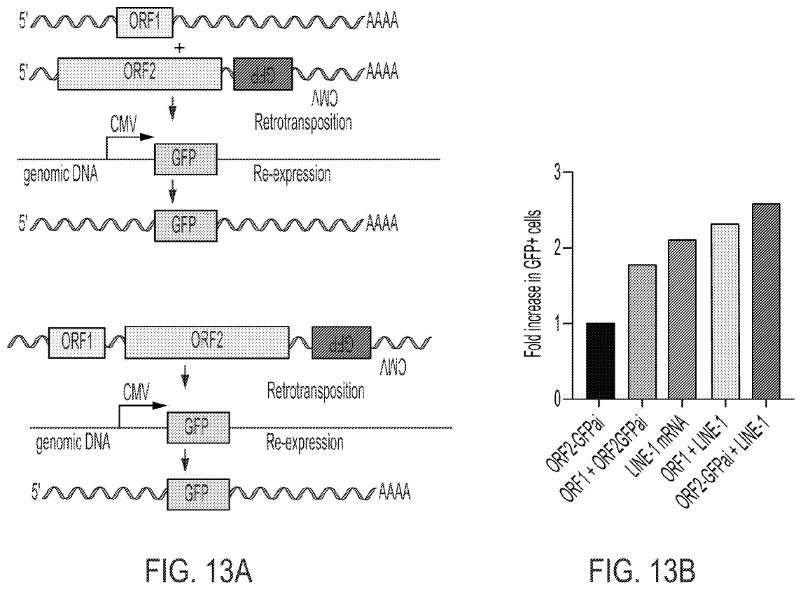

A complete LINE-1 mRNA encoding both ORF1 and ORF2 and GFP transgene in antisense orientation in a single mRNA molecule (LINE 1-GFP mRNA construct) was tested for delivery and genomic integration in a cell. mRNA contains the bicistronic ORF1 and ORF2 sequence with a CMV-GFP sequence in the 3′UTR going from 3′-5′ (FIG. 13A). In this study the cargo size is 2.4 kb. As shown in FIG. 13B, upon retrotransposition of the delivered ORF2-cmv-GFP antisense (LINE-1 mRNA), third bar from left, cells expressed higher GFP compared to ORF1 and ORF2 being on separate mRNA molecules (graph bar 1, 2). Inclusion of ORF1 in a separate mRNA in addition to LINE-1 complete mRNA increased GFP expression over LINE-1 alone. Inclusion of ORF2+ GFP expectantly showed higher GFP which could be the contribution of the additional ORF2 with the GFP cargo encoding mRNA.

To test whether subsequent electroporation increases retrotransposition efficiency, cells were electroporation every 48 hours. GFP positive cells were assessed using flow after culturing for 24-72 hrs.

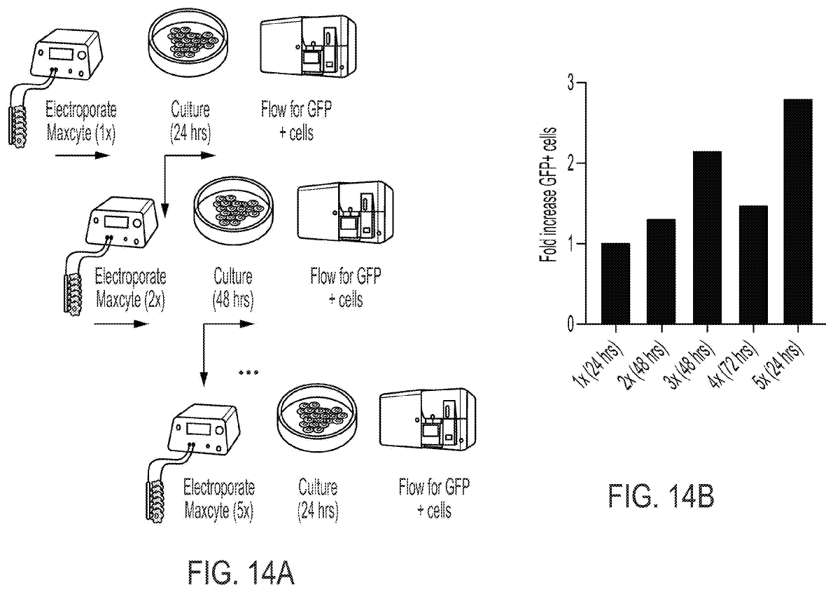

The fluorescence data were normalized to the values in the set with a single electroporation event. As shown in FIGS. 14A and 14B, multiple electroporation led to an upward trend in the expression of the transposed gene, but the changes were modest.

### Example 10. Modifications to the ORF2 Protein Sequence to Enhance Retrotransposition by mRNA

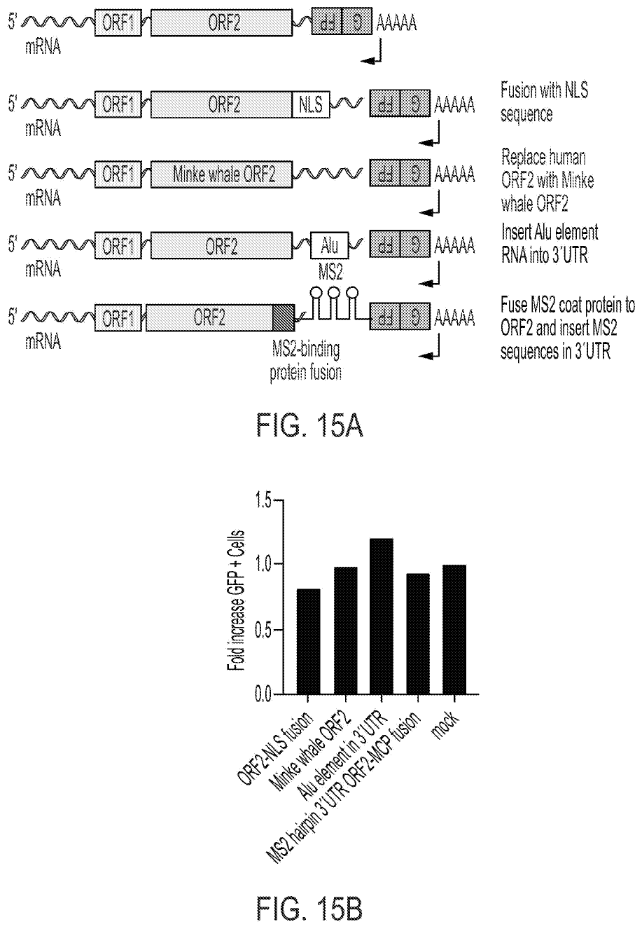

Modification of the LINE-1 sequence to enhance retrotransposition via mRNA delivery were tested using GFP reporter as readout. The experiment was performed as follows. All modifications were in the context of the bicistronic ORF1 and ORF2 sequence. (i) ORF2-NLS fusion was created by inserting C-terminal NLS sequence to the ORF2 sequence. (ii) Human ORF2 was replaced with Minke whale ORF2; (Ivancevic et al., 2016). (iii) Incorporation of an Alu element in the 3′UTR: Using a minimal sequence of the Alu element (AJL-H33A; Ahl et al., 2015) in the 3′UTR of the LINE-1. (iv) MS2-hairpin in the 3′UTR+ ORF2-MCP fusion: MS2 hairpins in the 3′UTR of the LINE-1 sequence and a MS2 hairpin binding protein (MCP) fused to the ORF2 sequence (FIG. 15A). The mock construct had the wild-type human ORF2 sequence.

Quantification of the fold increase in the fraction of GFP positive cells relative to mock construct electroporated cells are shown in FIG. 15B.

### Example 11. Retrotransposition in an Immune Cell

In this experiment, the inter-ORF region is further manipulated to determine if any of the changes improve GFP expression after transfection of the HEK cells. Taking LINE-lplasmid GFP, the inter-ORF region is manipulated as follows: (a) In one construct the inter-ORF region is replaced with an IRES from CVB3; (b) In another construct, the inter-ORF region is replaced with an IRES from EV71; (c) In three separate constructs, an E2A or P2A or T2A self-cleavage sequence is intercalated in the inter-ORF region.

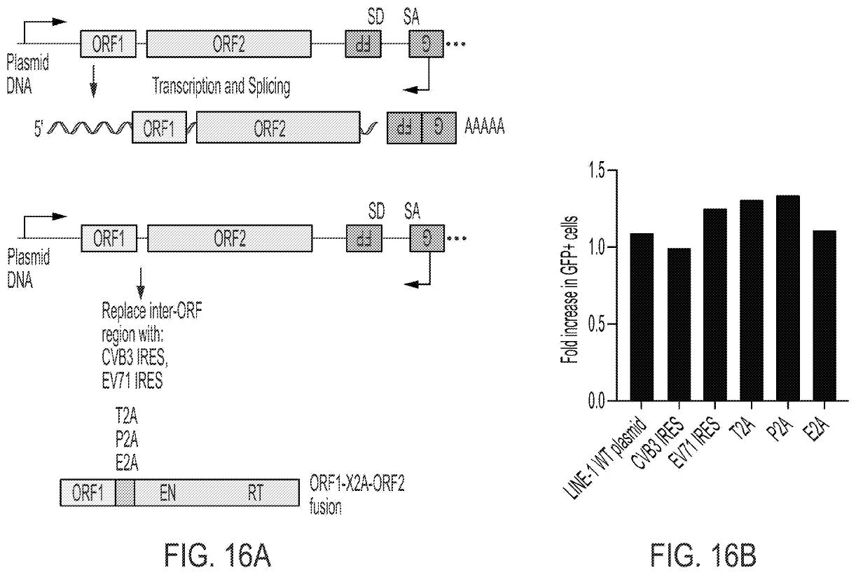

Result are as shown in FIG. 16. Compared to the LINE-1 plasmid GFP (LINE-1 wild type plasmid) led to only modest changes in the GFP readout, especially with T2A sequence insertion. Insertion of EV71 IRES sequence improved GFP expression, while CVB3 IRES did not show any improvement.

### Example 12. Retrotransposition in an Immune Cell

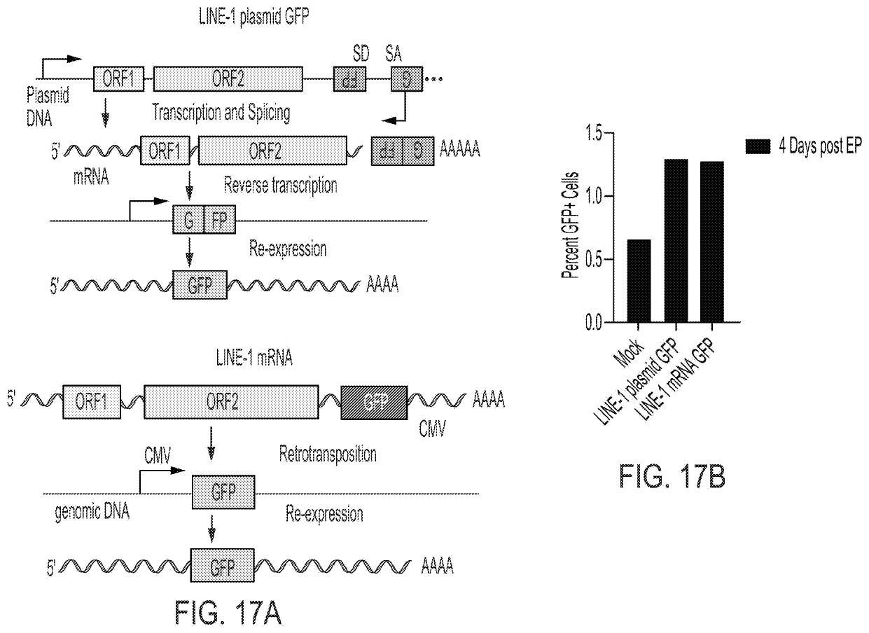

To test retrotransposition in immune cells, LINE-1 plasmid and mRNA were tested with the CMV-GFP antisense reporter cargo by electroporating into Jurkat cells, which is a T cell lymphoma line (FIG. 17A-FIG. 17B). Mock set were electroporated with a plasmid with no GFP sequence. GFP expression in the transfected cells was assessed, representative data at 4 days post electroporation is shown in FIG. 17B. Fold increase is reported relative to mock transfected cells. Both plasmid and mRNA delivery modes resulted in successful GFP expression.

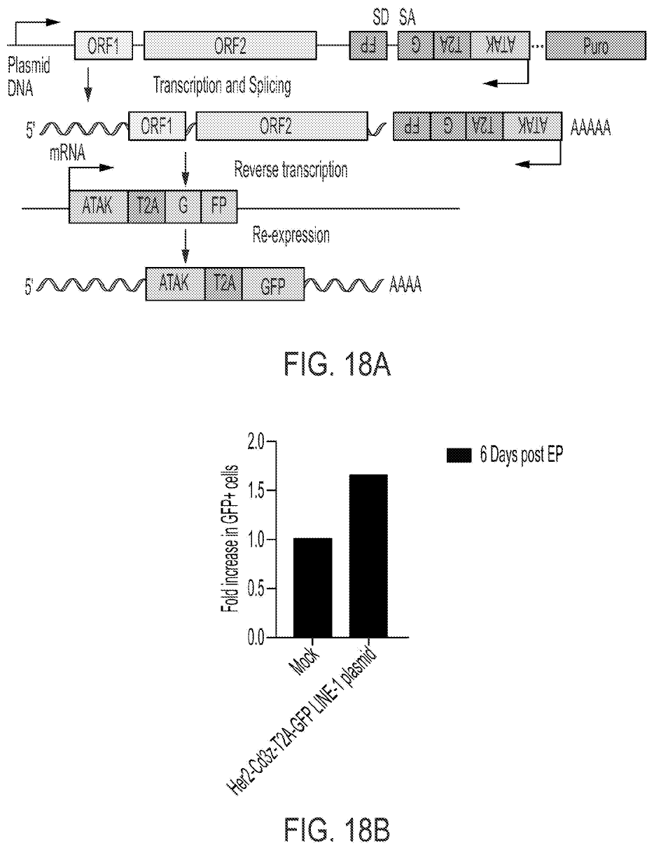

Next, THP-1 cells (a myeloid, monocytic cell line) were electroporated with a plasmid having LINE-1 sequences and a 3.7 kb cargo encoding a chimeric HER-2 binding receptor, and a split GFP (LINE-1 plasmid Her2-Cd3z-T2A-GFPintron) (FIG. 18A). The cargo is a chimeric receptor that comprises a HER2 binding extracellular domain, a CD3z transmembrane domain, and split GFP reporter. The plasmid was successfully integrated into the genome and showed prolonged expression, as demonstrated in FIG. 18B. Representative expression at day 6 post transfection is shown in the figure. From these studies, it was demonstrated that LINE-1 mediated gene delivery can result in successful stable genomic integration in various cell types, including epithelial cell types (HEK-293T cells); T cells (e.g., Jurkat cells); and cells of myeloid lineage (e.g., THP-1 cells) and results in prolonged expression. Moreover, unlike CRISPR dependent technologies such as Prime editing, retrotransposition can result in integration of large genetic cargo, and, these can be delivered as a single nucleic acid construct.

### Example 13. External Methods for Further Enhancing Efficiency of LINE-1 Mediated Retrotransposition of the Cargo Sequences

In this section, methods for further enhancing the efficiency of retrotransposition of cargo sequences into the genome of cells are detailed.

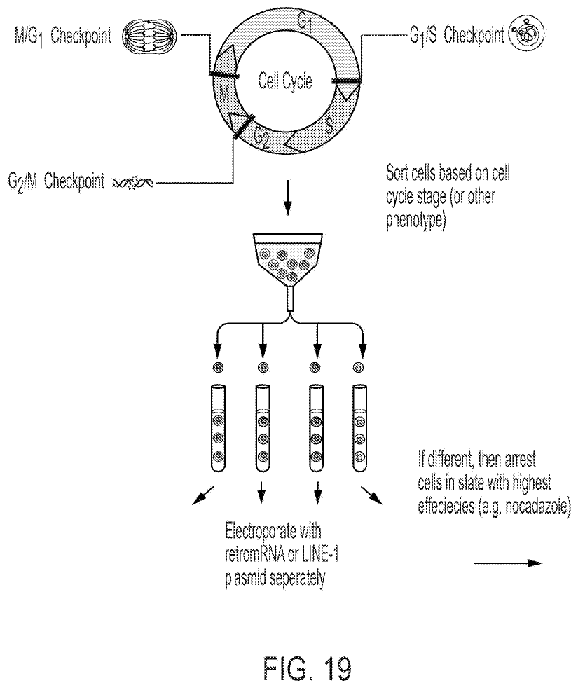

Cell cycle synchronization by selection of cells in a population that are in a certain stage of cell cycle or Gi arrest by a suitable agent can lead to higher nucleic acid uptake efficiency, e.g., plasmid vector transfection efficiency or electroporation efficiency. In this assay, cells are pre-sorted and each group is separately electroporated to ensure uniform electroporation. The efficiencies of electroporation are compared between these groups and a cell cycle stage that results in highest efficiency as determined by the expression of the GFP test plasmid or mRNA is selected (FIG. 19).

In another variation of this experiment, cells are synchronized with or without sorting by treating the cells, with a cell cycle arrest reagent for a few hours prior to electroporation. An exemplary list of cell cycle arrest reagents is provided in Table 1. The list is non-exhaustive, and is inclusive of reagents that can be proapoptotic, and hence careful selection suitable for the purpose and dose and time of incubation is optimized for use in the particular context.

TABLE 1
Exemplary non-exhaustive list of small molecule
reagents that are used for inhibiting cell cycle

| Agent | Cell cycle | Mechanism |
|---|---|---|
| 5-[(4- | Arrests cell cycle at | Inhibits c-Myc-Max |
| Ethylphenyl)methyl- | G0-G1 | dimerization |
| ene]-2-thioxo-4- | | |
| thiazolidinone | | |
| Itraconazole | Inhibits cell cycle at | SMO antagonist |
| | G1 | |
| ABT 751 (Tocris | Blocks cell cycle at | Inhibits microtubule |
| Bioscience, cat | G2M | proliferation |
| #4138) | | |
| Artesunate | Arrests cell cycle at | Suppresses ROS- |
| | G2M | induced NLRP3 |
| AZD 5438 | Blocks cell cycle at | Inhibits Cdk |
| | G2M, M, S and G1 | |
| | phases | |
| Baicalein | Arrests cell cycle at | Inhibits lipoxygenases |
| | G1 and G2 phases | |
| CPI 203 (alternative | Arrests cell cycle at | BET bromodomain |
| name: TEN 101) | G1 phase | inhibitor |
| Diadzein | Arrests cell cycle at G1 | Estrogen receptor agonist |
| DIM | Blocks cell cycle at | Induces EGFR activation |
| | G2M | |
| Epothilone B | Arrests cell cycle at | Inhibits tubulin |
| | G2M | proliferation |
| Indirubin-3′-oxime | Antiproliferative | Inhibits GSK3b |
| MPC 6827 | Cell cycle arrest | Inhibits microtubule |
| hydrochloride | | proliferation |
| Pladienolide | Inhibits G1 and G2/M | Decreases mRNA |
| | | splicing |
| Plumbagin | Induces G2/M arrest | Inhibits TOR signaling |
| | | and others |
| Temsirolimus | Induces G1/S | mTOR inhibitor |
| Toceranib | Cell cycle arrest | Inhibits PDGFR and |
| | | VEGFR |
| WYE 687 | Induces G1 arrest | mTOR inhibitor |
| dihydrochloride | | |
| YC1 | Induces G1 arrest | Guanylyl cyclase |
| | | activator |

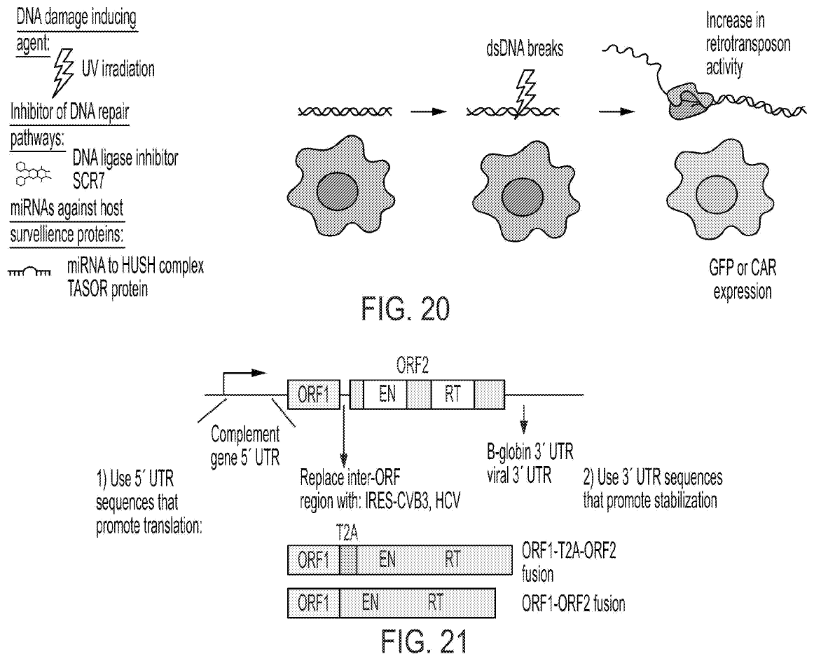

For certain ex vivo usages, retrotransposition is enhanced by inducing DNA double stranded breaks (DSB) in a cell that expresses a retrotransposition machinery as described in any of the examples above by controlled irradiation, which create opportunities for the homologous recombination and priming for the reverse transcriptase (FIG. 20). In another example, cells transfected with LINE-1 plasmid GFP construct and subjected to an irradiation pulse. GFP expression is monitored. The intensity and time of irradiation is optimized for obtaining the maximum benefit, as indicated by higher GFP expression.

In another example, cells transfected with LINE-1 plasmid GFP were divided into experimental sets that are treated as follows (i) irradiation in order to induce DSB (as described above); (ii) treat cells in this set with a small molecule, such as SCR7, that blocks DNA ligase and therefore inhibits the DNA damage repair machinery. Preventing protective repair mechanism from inhibiting the progress of the retrotransposition is expected to enhance GFP expression: (iii) irradiate the cells then treat the cells with SCR7, combination of the two is expected to show a more robust effect. GFP expression is monitored over a period of 6 days, and the set that shows maximum GFP fluorescence over the longest period indicates a condition that is adopted in further studies.

### Example 14. Enhancing Efficiency of LINE-1 Mediated Retrotransposition of the Cargo Sequences by Further Modification of the Construct

I. Enhancing non-coding regions of the construct to offer stability and higher expression. In this example a LINE-1 plasmid-GFP is further modified to test for increased GFP expression as follows: (a) In one construct, the 5′UTR is replaced with an UTR of a complement gene; (b) In another construct, the 3′ UTR is replaced with the UTR sequence of B-globin gene for increased stability; (c) In another construct the inter-ORF region is replaced with an IRES from CVB3; (d) In another construct, the inter-ORF region is replaced with an IRES from EV71 (e) In three separate constructs, an E2A or P2A or T2A self-cleavage sequence is intercalated in the inter-ORF region as shown in a diagrammatic representation in FIG. 21. In addition to the above, various combinations of (a)-(e) and additional combinations listed in Table 2 are tested using the same set-up as above. GFP expressions are monitored after transfection of the constructs in parallel test sets into HEK293T cells to see if any of these constructs increased GFP expression compared to the LINE-1 plasmid GFP alone. The combinations that show improvement are adopted.

TABLE 2
Exemplary combinations of 5′ and 3′ UTR and
inter-ORF insertion elements for inclusion in the LINE-1
construct for increase in retrotransposition efficiency.

| 5′-UTR sequences | 3′-UTR | Inter-ORF |
|---|---|---|
| selected from sequences | sequences | sequences |
| Complement 5′UTR | WPRE | T2A, E2A, P2A |
| Covid-19 5′ leader sequence | B-globin 3′UTR | CVB3 IRES |
| CYBA 5′UTR | RSV RSE | EV71 IRES |
| CYP2E1 5′ UTR | AREs | EMCV IRES |
| | RNA zipcodes for the | PV IRES |
| | ER | CSFV IRES |
| | mtRNR1-AES | HRV2 IRES |
| | | AAA (tri alanine |
| | | fusion or any |
| | | fusion-linker |
| | | sequence) |

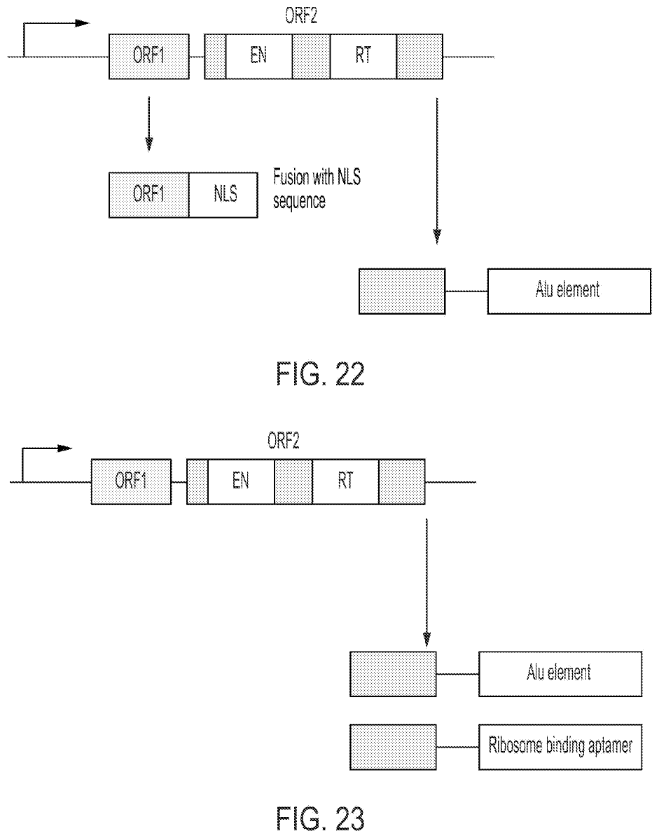

II. Enhancing localization and retention of the ORFs in the nucleus. In this example, LINE-1 plasmid-GFP is further modified to test for increased GFP expression as follows: (a) the ORF2 encoding sequence is fused with a nuclear localization sequence (NLS) (graphically represented in FIG. 15A second construct from top). (b) the ORF1 encoding sequence is fused with a nuclear localization sequence (NLS), graphically represented in FIG. 22; and (c) An Alu binding sequence is inserted 3′ of the sequence encoding ORF2 reverse transcriptase (graphically represented in FIG. 15A, fourth construct from the top; (d) Both (a) and (c) together (not shown); (e) Both (b) and (c) together, the NLS sequence is fused to the ORF1 N-terminus, and an Alu binding sequence is inserted 3′ of the sequence encoding ORF2 reverse transcriptase (FIG. 22) and (f) Integrating a SINE-derived nuclear RNA LOcalizatIoN (SIRLOIN) sequence in LINE-1 3′ UTR. HEK-293T cells were transfected with constructs (a)-(f) and the LINE-plasmid GFP construct in parallel. GFP expression is monitored after transfection into HEK293T cells. The set that shows maximum GFP fluorescence over the longest period is adopted.

III. Modifying construct to increase LINE-1-protein-RNA complex binding to the ribosome. In this example, an additional sequence is inserted in the 3′UTR of the LINE-1 construct to increase association of the LINE-1 protein RNA construct to the ribosomes, the sequence is an Alu element, or a ribosome binding aptamer (FIG. 23).

For enhancing LINE-1 protein-RNA complex binding to the ribosome, insertion of the following elements in the 3′ UTR of the mRNA is done and tested similar to the experiments above. Insertion of Alu elements is described above. In separate constructs, Alu element truncations, Ribosome binding aptamers (109.2-3) and Ribosome expansion segments (ES9S) binding sequence are inserted and each tested for increase in GFP expression.

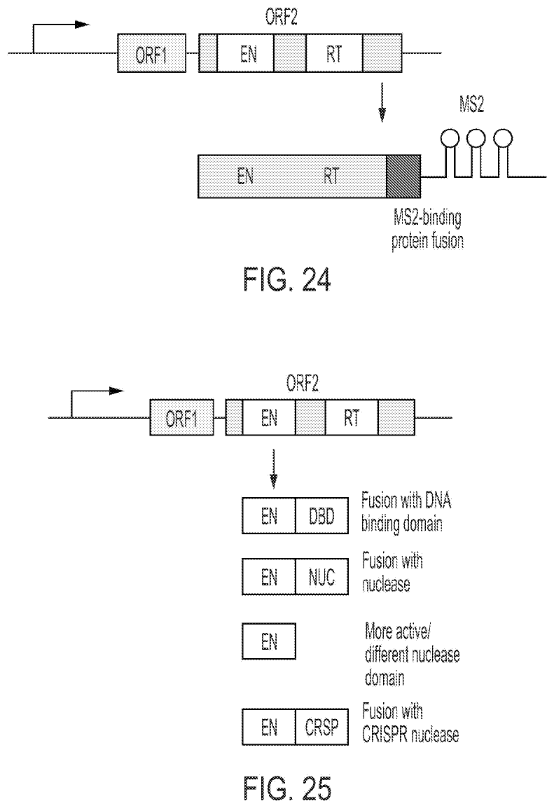

IV. Enhancing binding of ORF2 to its own mRNA for retrotransposition. In this example, a sequence containing MS2 binding loop structure is introduced into the 3′UTR of the LINE-1, and a sequence encoding MS2 RNA binding domain is fused to the RNA binding domain of the ORF2p-RT (graphically represented in FIGS. 4A and 4B, and FIG. 24, construct SEQ ID NO: 15). The fused protein will specifically attach to the MS2-binding structural motif in the 3′ UTR, and therefore any non-specific binding and retrotransposition is minimized (FIG. 24). GFP expression is monitored after transfection into HEK293T cells. Following a similar design, the ORF is fused with the protein binding sequences shown in left column of Table 3 below, combined with a cognate sequence inserted in the 3′UTR region of the ORF2 shown in the corresponding right column in the same row.

TABLE 3
Exemplary list of elements to enhance translation
efficiency and stability of the LINE-1 proteins
and increased expression of LINE-1 proteins.

| Elements to be fused | 3′ UTR sequence |
|---|---|
| with the LINE-1 ORF2 | recognizable by the element |
| PP7 coat protein | PP7 |
| Streptavidin | S1m aptamer |
| Tobramycin | Tobramycin aptamer |

V. Modifying the endonuclease function of the retrotransposon. In this example, the constructs are modified to test increase in GFP expression as follows. In a first experimental set, the LINE-1 plasmid GFP is cut at the 3′end of the endonuclease coding sequence of ORF2, and a sequence encoding the DNA binding domain (DBD) of a heterologous zinc finger protein (ZFP) is inserted. In another experimental set, the endonuclease domain is fused with a CRISPR nuclease. A variety of nucleases can be tested by modifying the LINE-1 plasmid GFP ORF by creating a fusion protein using DNA binding domains and cleavage domain as shown in a non-exhaustive list in Table 4, In addition, two ORF-2 domains are encoded in one set to facilitate dimerization. The construct that has higher GFP expression than the ORF2 endonuclease can be further selected. The plasmid designs are graphically represented in FIG. 25. GFP expression is monitored after transfection of the plasmids into HEK293T cells, and the set that yielded best.

TABLE 4
Exemplary non-exhaustive list of additional DNA cleavage domains/enzymes that can be fused
to or inserted in place of LINE-1 endonuclease.

| Gene/Enzyme | Description |
|---|---|
| Fokl | Class II endonuclease from Flavobacterium okeanokoites, recognition and |
| | cleavage sequence are separated by a few nucleotides; recognizes DNA |
| | sequence 5-GGATG-3′ |
| Restriction enzymes, | e.g., HindII, EcoR1, BamH1 |
| LAGLIDADG family | Intron encoded homing proteins found in various genera including |
| nuclease A | bacteria |
| (″LAGLIDADG″ | |
| disclosed as SEQ ID | |
| NO: 66) | |
| GIY-YIG | This domain is found in the amino terminal region of exonuclease abc |
| | subunit c (uvrC), bacteriophage T4, endonuclease segA, segB, seg C, |
| | seg D, and seg E and group I introns of fungi and phage. |
| His-Cys box | Homing endonucleases containing two clusters of conserved histidine and |
| | cysteine residues over a 100 amino acid region. |
| H-N-H | Widely present nuclease in phage DNA. Crucial component of the |
| | terminase packaging reaction of E. coli phage HK97. |
| PD-(D/E)xK | Phosphodiesterases, present in a large number of proteins, e.g., DUF4420, |
| | DUF3883, DUF4263, COG5482, COG1395, Tsp45I, HaeII, Eco47II, |
| | ScaI, HpaII. |
| Vsr-like/EDxHD | C-terminal nuclease domain that displays recognizable homology to bacterial |
| | Very short repair (Vsr) endonucleases |

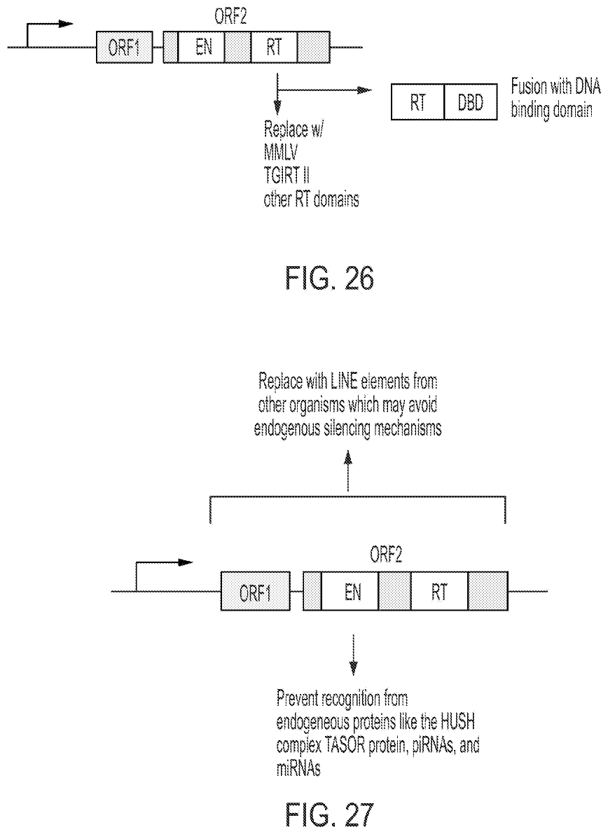

VI. Modifying the reverse transcriptase function of the retrotransposon. In this example, the reverse transcriptase domain of ORF2 is modified for increasing its efficiency. In one experimental set, the sequence encoding the human ORF2 in LINJE-lplasmid GFP is excised and replaced with a sequence encoding MMLV or TGIRTII In another experimental set, the ORF2 reverse transcriptase domain is fused with a DNA binding domain of a heterologous protein. The reverse transcriptase domains and/or the DNA binding domains can be selected from a non-exhaustive list provided in Table 5A-Table 5B. The constructs are graphically exemplified in FIG. 26. GFP expression is monitored after transfection into HEK293T cells.

TABLE 5A
Selected non-exhaustive list of reverse transcriptase for replacing the LINE-1 RT for
higher efficiency

| Reverse Transcriptase | Description |
|---|---|
| M-MLV-RT | Murine leukemia virus |
| TGIRT-II | Thermostable group II intron reverse transcriptase with high |
| | fidelity and processivity |
| AMV-RT | Avian Myeloblastosis Virus reverse transcriptase |
| Group II intron maturase | Derived from Eubacterium rectale |
| RT | |
| HIV-RT | Efficient RT derived from HIV |
| TERT | Catalyzes the RNA-dependent extension of 3′-chromosomal termini |
| | with the 6-nucleotide telomeric repeat unit, 5′-TTAGGG-3′. |

TABLE 5B
Selected non-exhaustive list of DNA-binding
domains for fusing to a RT for higher efficiency
DNA binding domains (DBD)

Zinc finger domains
Leucine zipper (bZip)
Helix-turn-helix domain
HMG-box
R2 retroelement DBD
Sso7d
Protein A (ssDNA)
OB-fold (ssDNA)

VII. Reesacing human LINE-]with LINE-]from other organisms. In this example, the sequence encoding human LINE-i is replaced by a LINE-i from a different organism. In one example, the human LINE-1 construct is compared with a construct where the human LINE-i is replaced by a minke whale LiNE-i sequence (FIG. 27). Using the same experimental framework, a number of ORFs are tested. An exemplary non-exhaustive list is provided in Table 6 below. A further comprehensive list is available in Ivancevic A. et al., Genome Biol Evol 8(1 1):3301-3322.

TABLE 6
Exemplary LINE-1 elements from organism
for use in replacement of the human LINE-1

| | No of total LINE-1/ |
|---|---|
| | No active/percent |
| Species Name | active |
| Balaenoptera | 8,012/5,006/62.4% |
| acutorostrata | |
| scammoni | |
| Rhinopithecus | 11,115/2,954/26.5% |
| roxellana | |
| Mus musculus | 18,280/4,143/22.66% |
| Aedes aegypti | 519/184/35.4% |
| Zea mays | 744/165/22.17% |
| Brassica napus | 1,929/565/29.2% |
| Brassica rapa | 543/228/41.9% |
| Danio rerio | 590/268/45.4% |

In another set, human LINE-i is retained as in the GFP plasmid, but an inhibitor of human LINE-1 silencer is utilized to prevent recognition by endogenous proteins like HUSH complex TASOR protein. In this case, the TASOR inhibitor is an inhibitory RNA, such as a miRNA.

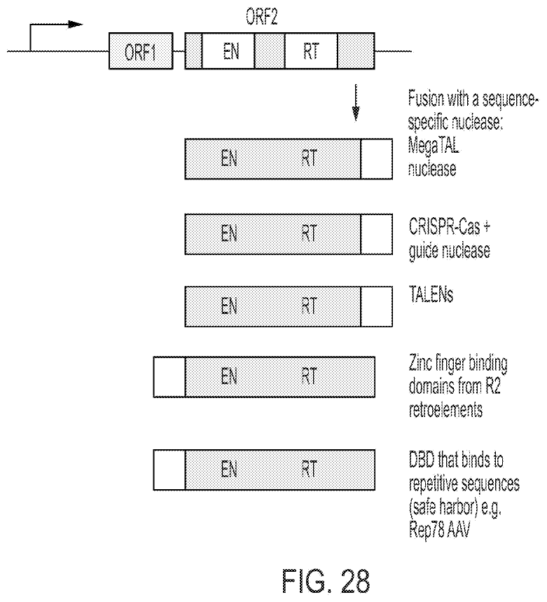

VIII. LINE-i fusion proteins for target specificity. In this example, the LINE-i plasmid GFP ORF2 is fused with a domain of a MegaTAL nuclease, a CRISPR-CAS nuclease, a TALEN, R2 retroelement binding zinc finger binding domain, or a DNA binding domain that can bind to repetitive elements such as Rep78 AAV. FIG. 28 exemplifies the deigns. Table 7 provides a list of the different elements that can be fused to increase sequence specific retrotransposition.

TABLE 7
Exemplary proteins with DNA binding
domains to be fused to ORF2 for increasing
retrotransposition specificity
Elements

Transcription Factors
MegaTAL nucleases
TALENs
Zinc finger binding domains from other retroelements
Safe harbor binding proteins
Cfp1

Each plasmid is transfected into HEK293 cells and GFP expression is monitored.

The modifications described in this section under (I)-(VIII) are designed to test for increase in retrotransposition efficiency, using GFP as readout. Following this, a number of useful modifications from (I)-(VIII) are incorporated into a single retrotransposition construct, tested with GFP as insert for the outcome, and the GFP sequence is replaced by the desired insert sequence.

### Example 15. Delivering a Large Payload for Prolonged Expression Using Retrotransposon Technology

Provided here are exemplary demonstrations of retrotransposon constructs are versatile for incorporating nucleic acid payloads into the genome of a cell and expressing an exemplary transgene. Retrotransposon constructs were designed as elaborated elsewhere in the disclosure.

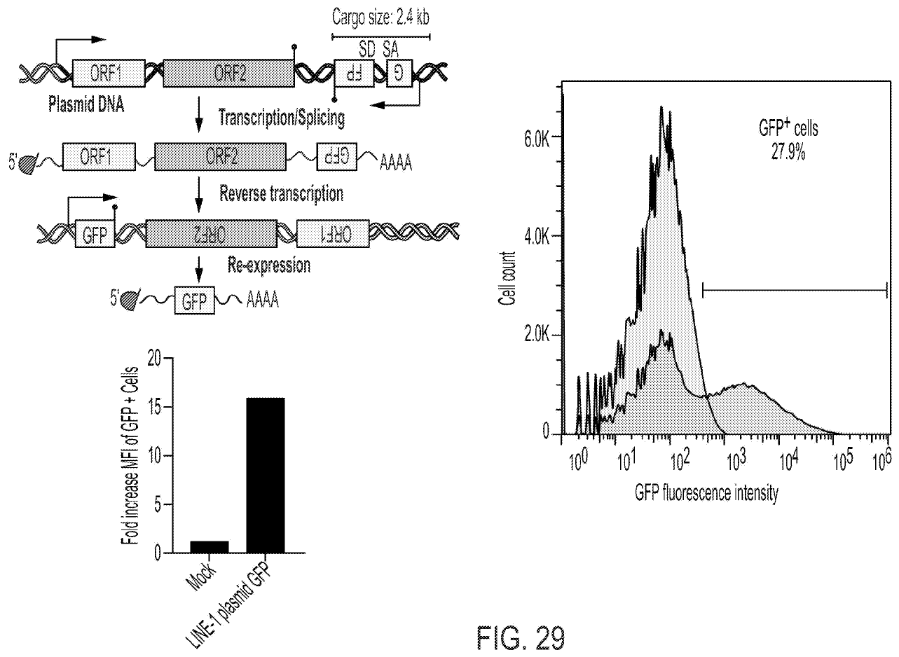

Briefly, in one set of validation experiments, GFP encoding payloads were constructed as follows: an antisense promoter sequence under doxycycline inducible control followed by antisense GFP gene split with an intron in the sense direction was placed downstream of the LINE-1 ORFs (FIG. 29). Splicing donor (SD) and splicing acceptor (SA) sequences are recognized and spliced out only when the mRNA is produced from the promoter in the top strand, therefore only the GFP gene integrated into genome from spliced mRNA generates fluorescent signal. As shown in the representative flow cytometry data in FIG. 2, the GFP expression was measured 35 days post doxycycline induction of the ORF expression using flow cytometry (green histogram) compared to a negative control plasmid (grey histogram). In this case, the cargo size was 2.4 kb.

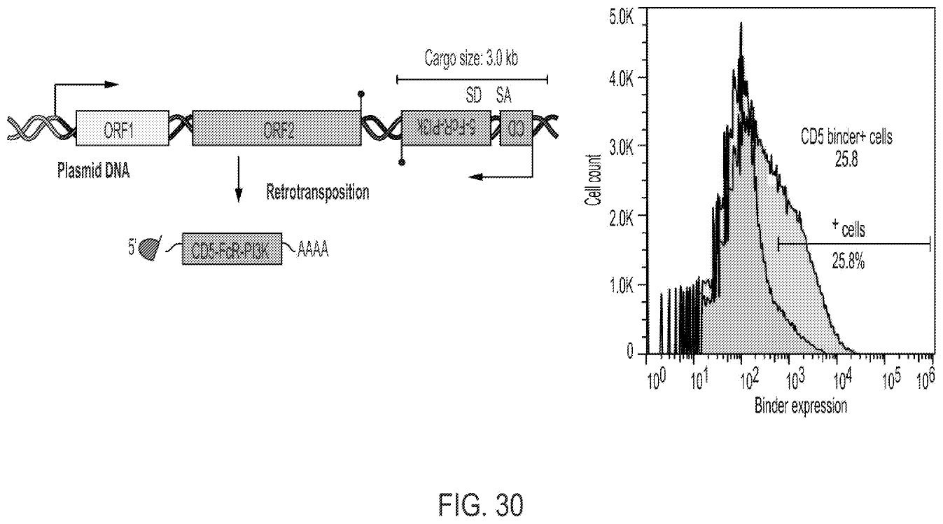

The cargo GFP gene in the previous construct was replaced with intron interrupted CD5-FcR-P13K CAR-M sequence (Morrissey et al., 2018). The CD5 binder expression was measured by flow cytometry using a Alexa647-conjugated CD5 protein such that retrotransposed cells are CD5-AF647 positive (red histogram) compared with a plasmid transfected negative control cell population (grey histogram) (FIG. 30). Successful expression of the 3.0 kb construct was demonstrated as shown in the figure.

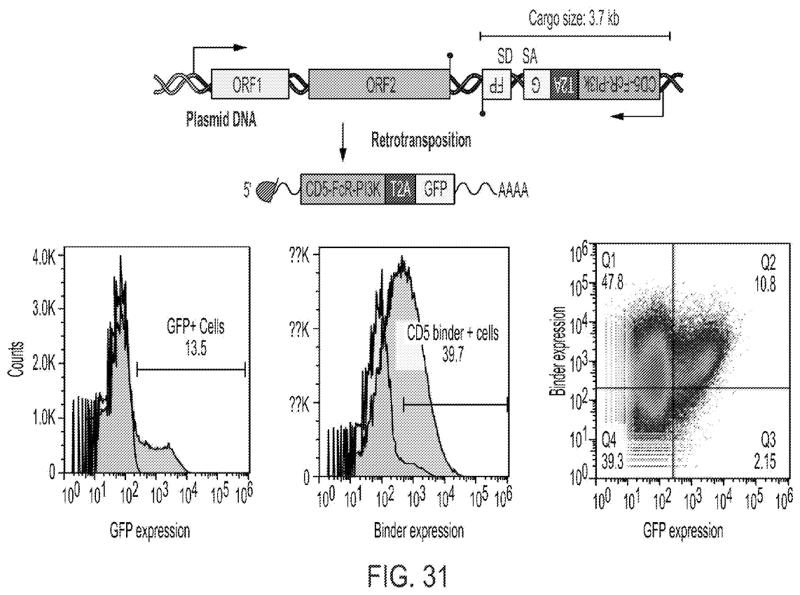

The cargo gene length was extended by adding the intron-interrupted GFP gene after the T2A sequence downstream of the CD5-FcR-PI3K CAR-M sequence (FIG. 31). The CD5 binder expression was measured by flow cytometry using a Alexa647-conjugated CD5 protein. The CD5 binder positive cells shown by red histogram, in comparison with a negative control (grey histogram). The GFP expression is measured using flow cytometry (green histogram) compared to a negative control plasmid transfected cells (grey histogram). The flow cytometry signal in the Q2 showed that 10.8% cells express both CAR-M and GFP proteins.

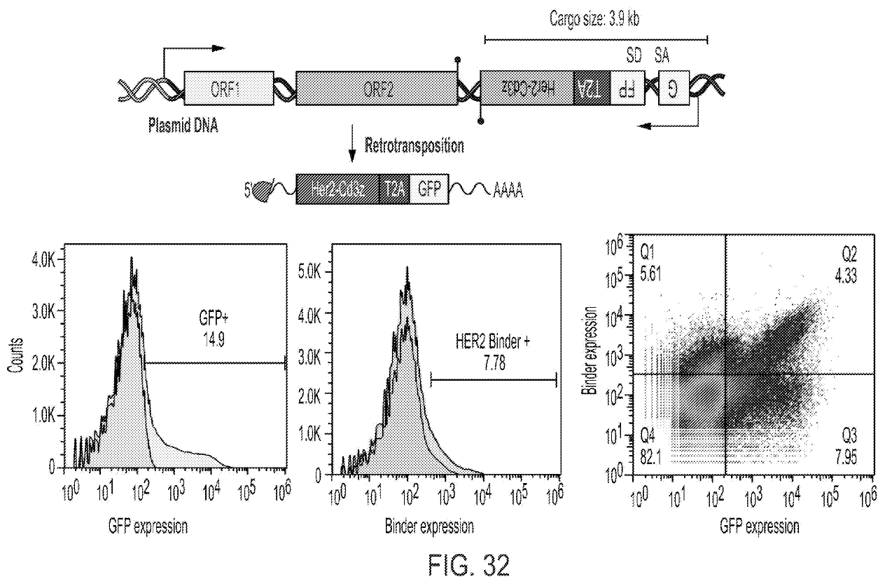

As shown in FIG. 32, the payload size limit has not been reached with retrotransposon delivery and integration (Retro-T delivery) with a 3.9 kb payload. The delivery mechanism described here was successful for expression of the first generation CART construct and GFP (separated by T2A site). In this example, different constructs were tested for retrotransposition efficiency of the insert sequence.

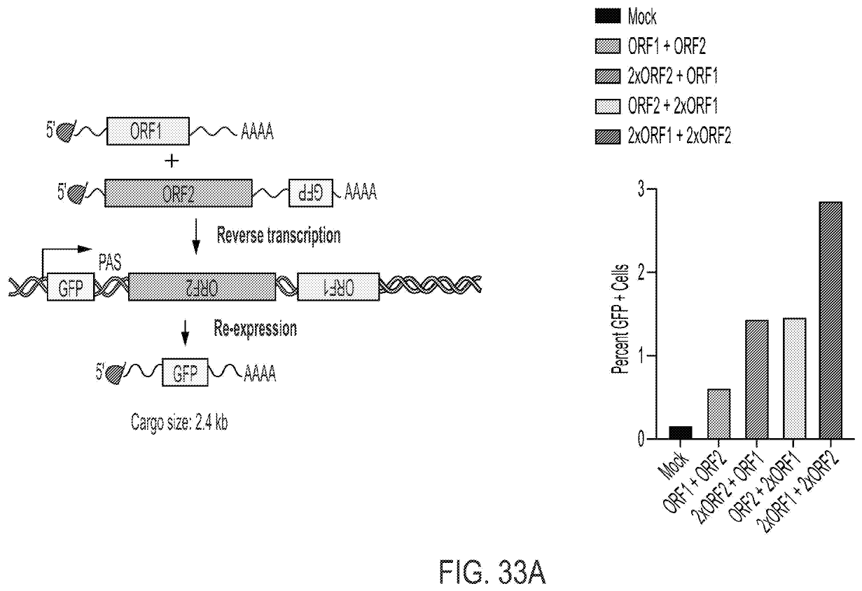

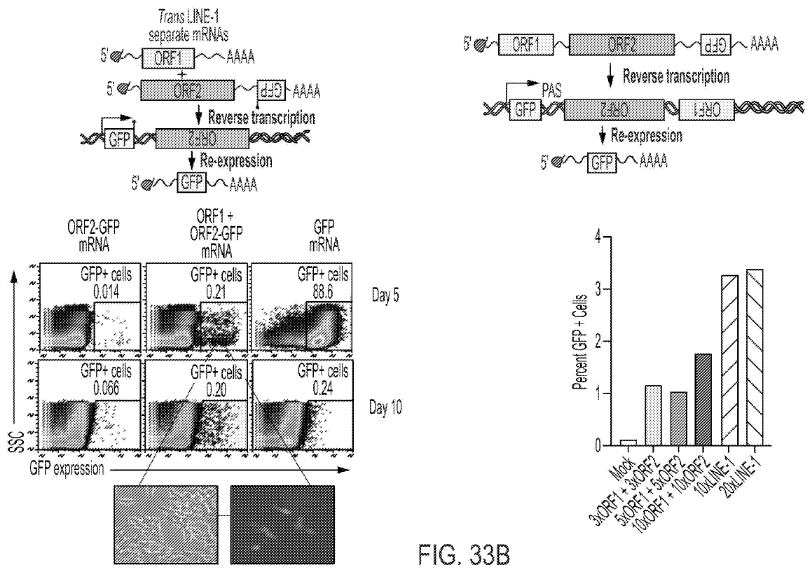

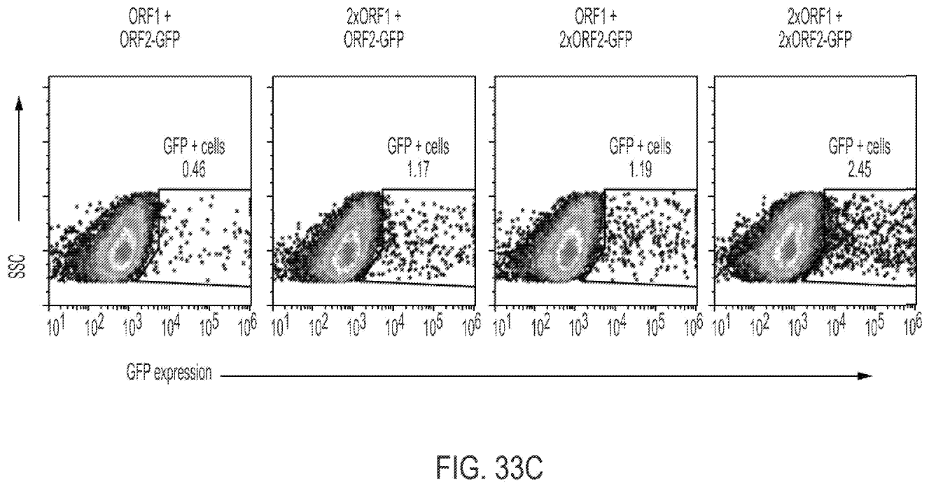

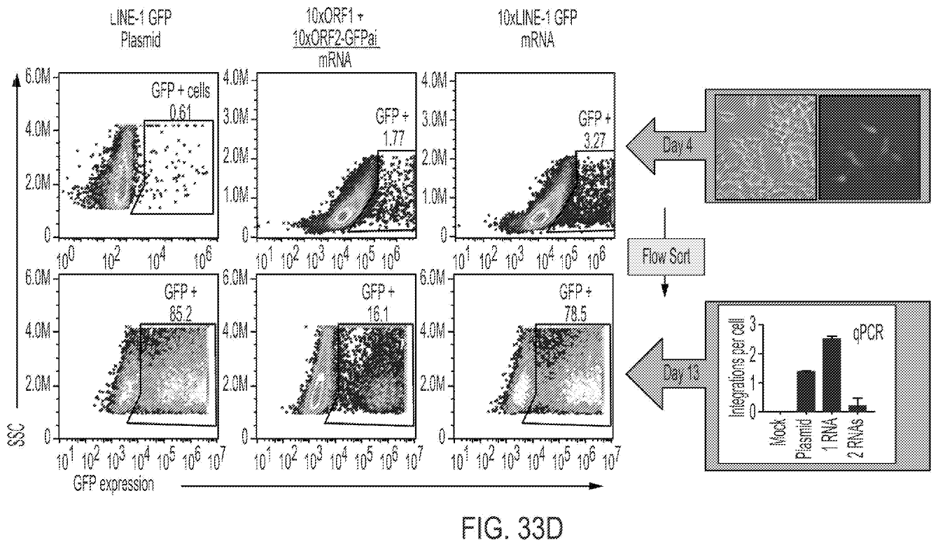

FIG. 33A shows gene delivery as mRNA results in successful integration. This data is the first to show that Retro T can be delivered as mRNA. A trans strategy of using separate mRNAs encoding for ORF1 and ORF2 with antisense promoter and GFP cargo (ORF2-GFPai) in the 3′ UTR for gene delivery was explored, as exemplified graphically in FIG. 33B (top panel). FIGS. 33B-33D demonstrate experimental results from multiple representative assays. Separate mRNAs that expression the LINE-1 proteins could reconstitute the RNA-protein complex required for retrotransposition. The cis strategy uses a single bicistronic LINE-1 mRNA with the antisense promoter and GFP gene cargo in the 3′UTR. Constructs comprising variable amounts and proportions of ORF2 and ORF1 were compared as shown in FIG. 33B and FIG. 33C with GFP encoding sequence as payload. FIG. 33D shows that introducing a single mRNA yields higher number of integrations per cell. Sorting of 293T GFP cells to enrich for retrotransposed cells for biochemical and integration assays. Cells are the same as in FIG. 33B and show GFP expression 4 days post-sort in bottom panels. The graph shows qPCR assay for genomic DNA integration from different LINE-1 plasmid transfected, LINE-1 mRNA (retro-mRNA), and ORF1 and ORF2-GFP mRNA electroporated cells. Two qPCR primer-probe sets were used, one for the housekeeping gene RPS30 and the other for the GFP gene. Plasmid-transfected cells use a plasmid that does not contain and SV40 maintenance sequence. Integration per cell is calculated from determining copy numbers per samples through interpolation of a standard curve of plasmid and genomic DNA and normalizing for the two copies of RPS30 per 293T cell. Error bars denote standard deviation of three technical replicate measurements.

### Example 16. Delivery to Diverse Cell Types

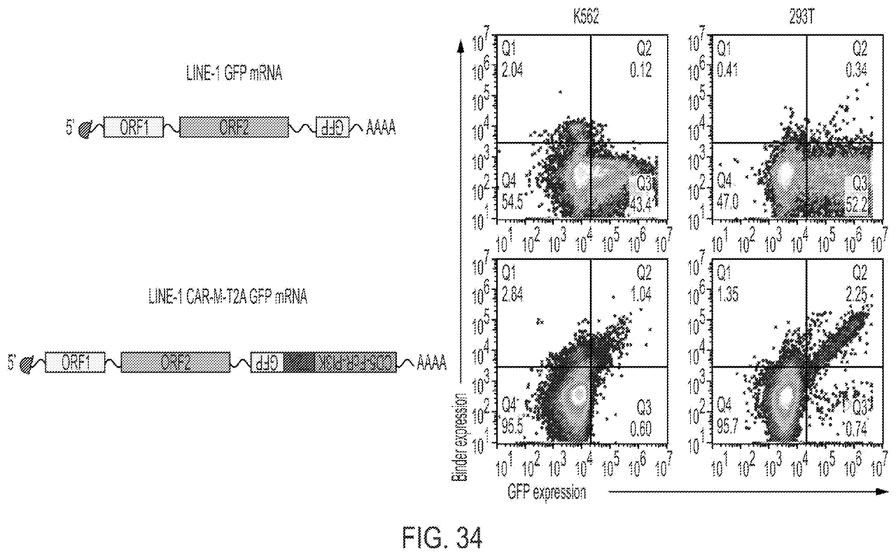

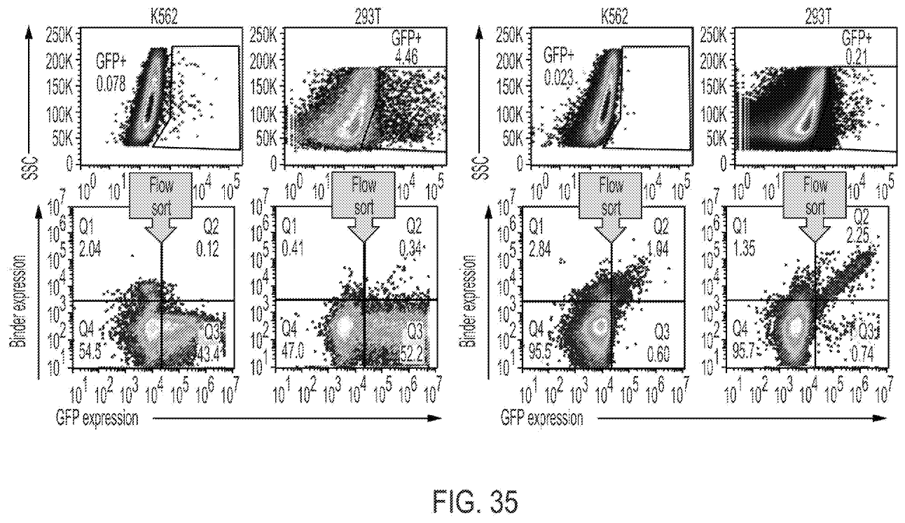

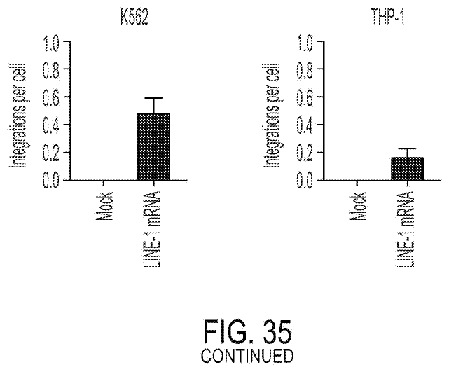

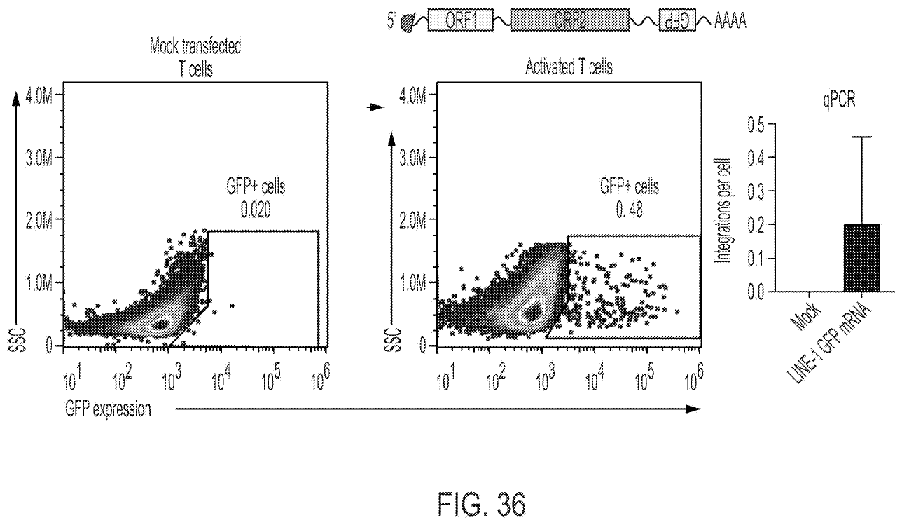

As shown in FIGS. 34-38, the mRNA constructs comprising a gene of interest, e.g. encoding a CAR protein, or for example, a GFP protein can be efficiently expressed in diverse cell types, such as epithelial cells (e.g., HEK 293 cells), monocytic cells lines (e.g., THP-1 cells), lymphoblastic cell lines (e.g., K562 cells), and primary lymphocytes (T cells). Activated primary T cells were also successfully transfected with mRNA with genomic integration and expression of GFP (FIG. 36). Primary T cells were isolated and expanded using IL7/IL 15; and a 1′^{t} Gen CAR construct was delivered on day 2 post activation.

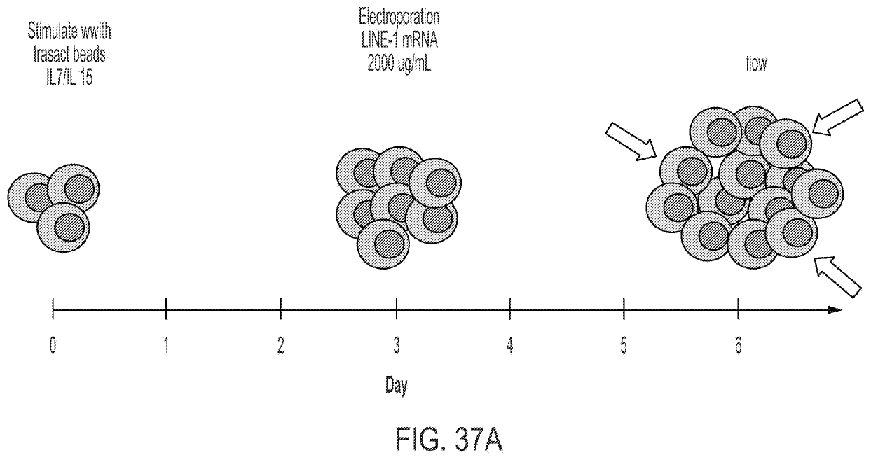

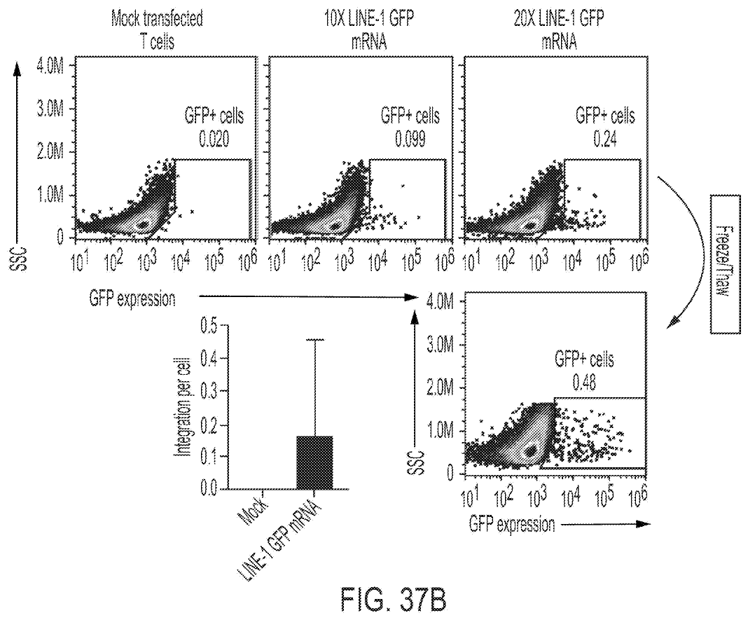

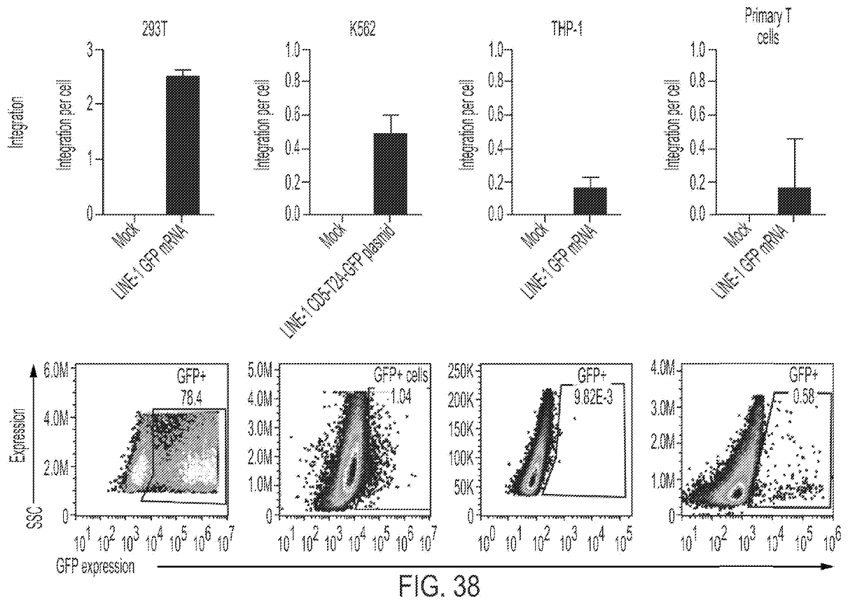

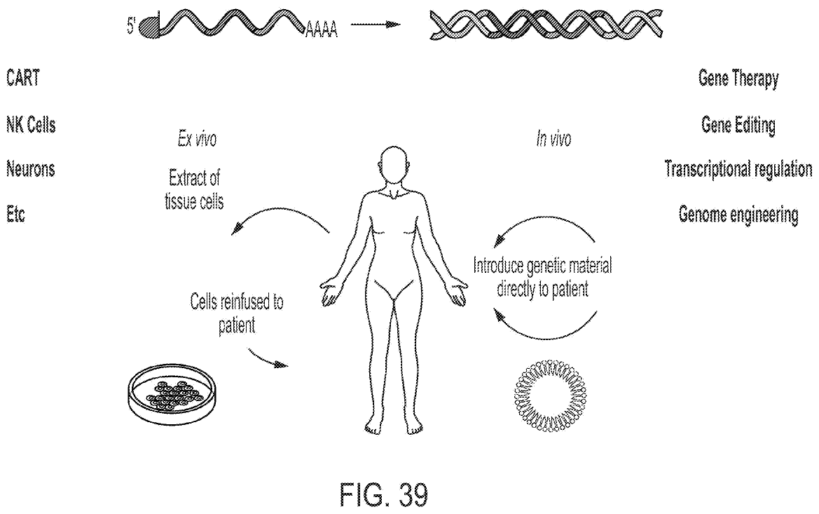

Cells sorted and frozen. GFP expression was detectable after a freeze-thaw cycle (FIG. 37A-B). This indicates the versatile nature of mRNA mediated delivery and L1-transposon mediated integration. FIG. 38 shows a representative assay of GFP mRNA integration and expression in 293T cells, K562 cells, THP-1 cells and Primary T cells.

### Example 17. Modifying the Retrotransposition System, Exchanging for Site-Specificity of an Integrase for Genomic Integration

In this prophetic example, large mRNA constructs are stably incorporated and expressed in non-dividing mammalian cell using the retrotransposon integration system into the genome of the cell with a high degree of location specificity using serine integrases. Retrotransposon constructs are designed as elaborated elsewhere in the disclosure.

In this example a first construct, an mRNA construct is designed to include Cas endonuclease fused with a serine integrase comprising a mutation in the catalytic domain that eliminates a possibility of double stranded polynucleotide integration by the integrase, and prime editing guide RNA (pegRNA) oligonucleotides directed to a specific genomic locus, e.g., a histone gene locus, an actin gene locus etc, depending on which the guide RNA is specifically designed. One or more T2A cleavage sites separate the coding sequence of the individual proteins, e.g. Cas9, and the serine integrase of the fragment thereof.

The pegRNAs are designed to insert a 41 bp AttB landing site. In one or more alternative designs, a LINE 1 ORF2 binding site is incorporated that is located conveniently and in proximity (e.g., juxtaposed) for binding of an ORF2 protein in relation to the integrase binding site.

A second mRNA construct is designed to include a LINE1 mRNA fused with a cargo sequence that is greater than 5, 6, 7, 8, 9, 10 kilobases, and encoding a protein; a mutated endonuclease, and an AttP attachment site fused to the cargo sequence for the integrase to attract and bring the cargo sequence to the landing sequence. The AttP attachment site is complementary to the AttB landing site.

The mRNA constructs are prepared through in vitro transcription and then purified. Each of resulting purified mRNA constructs are incorporated separately into lipid nanoparticles (LNP). Using electroporation, the two mRNA constructs are co-delivered using LNPs into cell. The Cas9 endonuclease fused to the pegRNAs is guided to insert the genomic landing sequence into the specific gene locus. The AttB landing site acts like a beacon that attracts for the AttP attachment site, which by design is comprised within the cargo sequence that is associated with the LINE1 mRNA. Co-translation of the proteins lead to generation of the ORF polypeptides, and led by the integrase and tethered by the attachment site, the ORF contacts with the associated ORF binding site at the proximity, and integrates the cargo at the specific site.

In yet another alternative design system, the integrase system is altogether bypassed and the PEG-RNA incorporates only a ORF binding site at a specific genomic locus directed by the guide RNA.

Site-specific integration is confirmed by Sanger sequencing. Flow cytometry is performed to demonstrate the expression of the insert sequence in the cell. Cell survival and genomic integration is found to be higher than often found in plasmid- or vector-based systems, as the mRNA has less of a detrimental impact on the cells.

### Example 18. Exemplary Sequences

Following are exemplary sequences of the constructs used in the examples. These sequences are for reference exemplary purposes and sequence variations and optimizations that are conceivable by one of skill in the art without undue experimentation are contemplated and encompassed by the disclosure.

Where mRNA sequences are referred in the sequence title, the construct recites nucleotides of a DNA template and one of skill in the art can easily derive the corresponding mRNA sequence.

TABLE 8
Plasmid and mRNA construct sequences

ORF1-FLAG-mRNA (Codon Optimized human
ORF1 coding sequence-FLAG) (SEQ ID NO: 35):
TAATACGACTCACTATAGGGAGAAAGACGCCACCATGGGCAAGAA
GCAAAATCGCAAGACGGGGAATTCCAAGACACAATCCGCTAGCCC
ACCACCTAAAGAGCGTTCTAGCTCCCCTGCTACTGAGCAGTCCTG
GATGGAAAACGACTTCGATGAACTCCGGGAAGAGGGATTTAGGCG
ATCCAACTATTCAGAACTCCGCGAAGATATCCAGACAAAGGGGAA
GGAAGTCGAGAATTTCGAGAAGAACCTCGAGGAGTGCATCACCCG
TATCACAAACACTGAGAAATGTCTCAAAGAACTCATGGAACTTAA
GACAAAAGCCAGGGAGCTTCGAGAGGAGTGTCGGAGTCTGAGATC
CAGGTGTGACCAGCTCGAGGAGCGCGTGAGCGCGATGGAAGACGA
GATGAACGAGATGAAAAGAGAGGGCAAATTCAGGGAGAAGCGCAT
TAAGAGGAACGAACAGAGTCTGCAGGAGATTTGGGATTACGTCAA
GAGGCCTAACCTGCGGTTGATCGGCGTCCCCGAGAGCGACGTAGA
AAACGGGACTAAACTGGAGAATACACTTCAAGACATCATTCAAGA
AAATTTTCCAAACCTGGCTCGGCAAGCTAATGTGCAAATCCAAGA
GATCCAACGCACACCCCAGCGGTATAGCTCTCGGCGTGCCACCCC
TAGGCATATTATCGTGCGCTTTACTAAGGTGGAGATGAAAGAGAA
GATGCTGCGAGCCGCTCGGGAAAAGGGAAGGGTGACTTTGAAGGG
CAAACCTATTCGGCTGACGGTTGACCTTAGCGCCGAGACACTCCA
GGCACGCCGGGAATGGGGCCCCATCTTTAATATCCTGAAGGAGAA
GAACTTCCAGCCACGAATCTCTTACCCTGCAAAGTTGAGTTTTAT
CTCCGAGGGTGAGATTAAGTATTTCATCGATAAACAGATGCTGCG
AGACTTCGTGACAACTCGCCCAGCTCTCAAGGAACTGCTCAAAGA
GGCTCTTAATATGGAGCGCAATAATAGATATCAACCCTTGCAGAA
CCACGCAAAGATGGATTATAAGGATGACGATGATAAATGA
(SEQ ID NO: 35)
ORF2-FLAG-GFP aim RNA (Codon Optimized
human ORF2 coding sequence) (SEQ ID NO: 36)
TAATACGACTCACTATAGGGAGAAAGACGCCACCATGACAGGTTC
AAATAGTCACATTACGATTCTCACTCTGAATATAAATGGGCTGAA
TTCTGCAATTAAACGGCACAGGCTTGCTTCCTGGATAAAGTCTCA
AGACCCCTCAGTGTGCTGTATTCAGGAAACGCATCTCACGTGCAG
GGACACCCATCGGCTGAAAATAAAAGGCTGGCGGAAGATCTACCA
AGCCAATGGAAAACAAAAGAAGGCTGGGGTGGCGATACTTGTAAG
CGATAAAACAGACTTTAAACCAACTAAGATCAAACGGGACAAAGA
GGGCCATTACATCATGGTAAAGGGTAGTATTCAACAAGAGGAGCT
GACTATCCTGAATATTTATGCACCTAATACTGGAGCCCCCAGATT
CATAAAGCAAGTGTTGAGTGACCTTCAACGCGACCTCGACTCCCA
CACTCTGATCATGGGAGACTTTAACACCCCGCTGTCCACTCTCGA
CAGATCTACTAGACAGAAAGTCAACAAGGATACACAGGAACTGAA
CAGTGCTCTCCACCAAGCGGACCTTATCGACATCTACAGAACACT
CCACCCCAAAAGCACAGAATATACCTTCTTTTCAGCCCCTCACCA
CACCTATTCCAAAATTGACCACATTGTGGGGAGTAAAGCCCTTCT
CTCCAAATGTAAACGGACCGAAATTATCACTAACTATCTCTCCGA
CCACAGTGCAATAAAACTTGAATTGCGAATTAAGAATCTCACTCA
AAGTAGATCCACGACATGGAAACTGAACAATCTCCTCTTGAATGA
CTACTGGGTGCATAACGAAATGAAGGCTGAAATAAAGATGTTCTT
TGAGACCAACGAAAACAAAGACACCACGTACCAGAATCTCTGGGA
CGCTTTCAAAGCAGTGTGTCGAGGAAAATTTATTGCACTGAATGC
TTACAAGCGGAAGCAGGAAAGATCCAAAATAGACACCCTGACTAG
CCAACTTAAAGAACTGGAAAAGCAAGAGCAAACTCATAGCAAAGC
TAGCCGTCGCCAAGAAATTACGAAAATCAGAGCTGAACTGAAGGA
AATTGAGACACAGAAAACCCTGCAAAAGATAAATGAAAGCCGCAG
CTGGTTCTTTGAACGCATCAACAAAATCGATAGGCCACTTGCTCG
CCTTATCAAGAAGAAAAGGGAGAAGAATCAAATCGACACTATAAA
GAATGATAAAGGCGATATAACCACCGATCCCACAGAAATTCAAAC
AACCATACGCGAATACTACAAACACCTCTACGCCAATAAACTCGA
AAATCTCGAGGAAATGGATACATTCCTCGACACGTACACCCTTCC
CAGGCTGAACCAGGAAGAAGTTGAATCACTGAATCGGCCTATCAC
GGGGAGTGAAATAGTAGCTATCATCAATTCACTCCCTACCAAGAA
GTCACCCGGACCTGATGGATTCACCGCCGAATTCTACCAGAGATA
CATGGAAGAACTGGTGCCCTTCTTGCTGAAACTTTTCCAAAGTAT
TGAGAAAGAGGGAATACTTCCAAACTCATTTTATGAGGCATCCAT
CATTCTGATCCCGAAGCCCGGCAGGGACACGACCAAGAAAGAGAA
TTTTCGACCAATCTCATTGATGAACATTGATGCAAAGATCCTCAA
TAAAATACTGGCAAATCGGATTCAGCAGCACATAAAGAAGCTGAT
CCACCATGATCAAGTAGGCTTCATCCCCGGTATGCAAGGTTGGTT
CAATATACGAAAATCAATCAATGTTATCCAGCATATAAACCGGGC
CAAAGACAAGAACCACATGATTATTAGTATCGATGCTGAGAAAGC
CTTTGACAAAATACAACAACCCTTCATGCTGAAAACATTGAATAA
GCTGGGAATTGATGGCACCTACTTCAAAATCATCAGAGCCATATA
TGACAAACCAACAGCAAATATCATTCTGAATGGTCAGAAATTGGA
AGCATTCCCCTTGAAAACCGGCACACGGCAGGGTTGCCCTCTGTC
ACCACTCCTCTTCAACATCGTGTTGGAAGTTCTTGCCCGCGCAAT
CCGGCAGGAAAAGGAAATCAAGGGCATTCAACTGGGCAAAGAGGA
AGTTAAATTGAGCCTGTTTGCAGACGACATGATCGTCTATTTGGA
AAACCCCATAGTTAGTGCACAAAATCTGCTGAAGTTGATCAGTAA
TTTCTCCAAAGTGAGTGGGTACAAAATCAATGTGCAAAAGAGCCA
AGCTTTCTTGTACACCAACAACAGGCAAACTGAGTCTCAAATCAT
GGGCGAACTCCCCTTCGTGATTGCATCCAAGCGGATCAAATACCT
GGGGATTCAATTGACTCGTGATGTGAAGGACCTCTTCAAGGAGAA
CTACAAACCCCTGCTCAAGGAAATCAAAGAGGACACAAACAAATG
GAAGAACATTCCATGCTCTTGGGTGGGAAGGATCAATATCGTCAA
AATGGCCATCCTGCCCAAGGTAATTTACAGGTTCAATGCTATACC
CATCAAGCTCCCCATGACATTCTTCACAGAACTTGAAAAGACGAC
GCTGAAGTTCATTTGGAACCAGAAACGTGCCAGGATTGCTAAATC
TATTCTCTCCCAAAAGAACAAAGCTGGCGGAATCACACTCCCAGA
CTTCAAACTTTACTACAAGGCGACCGTGACGAAAACGGCTTGGTA
CTGGTACCAAAACAGGGATATAGATCAATGGAACCGAACGGAGCC
CAGCGAAATTATGCCTCATATATACAACTATCTGATCTTTGACAA
ACCGGAGAAGAACAAGCAATGGGGAAAGGATAGTCTGTTTAATAA
ATGGTGCTGGGAAAACTGGCTCGCAATCTGTAGGAAGCTGAAACT
GGATCCATTCTTGACGCCTTATACAAAGATAAATTCCCGATGGAT
TAAAGATCTCAACGTGAAACCCAAAACAATTAAAACCCTCGAGGA
AAACCTGGGTATTACGATTCAGGACATTGGGGTGGGAAAGGACTT
CATGTCCAAAACCCCAAAAGCGATGGCAACCAAAGACAAAATCGA
CAAATGGGATCTCATAAAACTTAAGTCATTTTGCACAGCTAAAGA
AACGACAATTAGGGTGAACCGACAACCGACCACTTGGGAGAAAAT
CTTCGCAACATACAGTTCTGACAAAGGCCTGATTTCCAGGATCTA
CAATGAATTGAAACAAATTTACAAGAAGAAGACGAACAACCCTAT
AAAGAAATGGGCCAAGGACATGAACAGACACTTCTCTAAGGAAGA
CATTTATGCAGCCAAGAAACACATGAAGAAATGCAGCTCTTCACT
GGCAATCAGGGAAATGCAAATCAAAACAACAATGAGATATCATCT
CACACCCGTCAGAATGGCCATCATTAAGAAGAGCGGAAACAACCG
GTGCTGGCGTGGTTGCGGAGAAATCGGTACTCTCCTTCACTGTTG
GTGGGACTGTAAACTCGTTCAACCACTGTGGAAGTCTGTGTGGCG
GTTCCTCAGAGATCTGGAACTCGAAATCCCATTTGACCCAGCCAT
CCCTCTCCTGGGTATATACCCGAATGAGTATAAATCCTGCTGCTA
TAAAGACACCTGCACAAGGATGTTTATTGCAGCTCTCTTCACAAT
CGCGAAGACGTGGAACCAACCCAAATGTCCGACTATGATTGACTG
GATTAAGAAGATGTGGCACATATACACTATGGAATACTATGCTGC
GATCAAGAACGATGAGTTCATATCATTTGTGGGCACATGGATGAA
ACTCGAAACCATCATACTCTCTAAATTGAGTCAAGAACAGAAAAC
TAAACACCGTATATTTTCCCTGATCGGTGGGAATTAGCTACAAAG
ACGATGACGACAAGGACCATGGAGACGGTGAGAGACACAAAAAAT
TCCAACACACTATTGCAATGAAAATAAATTTCCTTTATTAGCCAG
AAGTCAGATGCTCAAGGGGCTTCATGATGTCCCCATAATTTTTGG
CAGAGGGAAAAAGATCTCAGTGGTATTTGTGAGCCAGGGCATTGG
CCTTCTGATAGGCAGCCTGCACCTGAGGAGTGCGGCCGCTTTACT
TGTACAGCTCGTCCATGCCGAGAGTGATCCCGGCGGCGGTCACGA
ACTCCAGCAGGACCATGTGATCGCGCTTCTCGTTGGGGTCTTTGC
TCAGGGCGGACTGGGTGCTCAGGTAGTGGTTGTCGGGCAGCAGCA
CGGGGCCGTCGCCGATGGGGGTGTTCTGCTGGTAGTGGTCGGCGA
GCTGCACGCTGCCGTCCTCGATGTTGTGGCGGATCTTGAAGTTCA
CCTTGATGCCGTTCTTCTGCTTGTCGGCCATGATATAGACGTTGT
GGCTGTTGTAGTTGTACTCCAGCTTGTGCCCCAGGATGTTGCCGT
CCTCCTTGAAGTCGATGCCCTTCAGCTCGATGCGGTTCACCAGGG
TGTCGCCCTCGAACTTCACCTCGGCGCGGGTCTTGTAGTTGCCGT
CGTCCTTGAAGAAGATGGTGCGCTCCTGGACGTAGCCTTCGGGCA
TGGCGGACTTGAAGAAGTCGTGCTGCTTCATGTGGTCGGGGTAGC
GGCTGAAGCACTGCACGCCGTAGGTCAGGGTGGTCACGAGGGTGG
GCCAGGGCACGGGCAGCTTGCCGGTGGTGCAGATGAACTTCAGGG
TCAGCTTGCCGTAGGTGGCATCGCCCTCGCCCTCGCCGGACACGC
TGAACTTGTGGCCGTTTACGTCGCCGTCCAGCTCGACCAGGATGG
GCACCACCCCGGTGAACAGCTCCTCGCCCTTGCTCACCATGGTGG
CGGGATCTGACGGTTCACTAAACCAGCTCTGCTTATATAGACCTC
CCACCGTACACGCCTACCGCCCATTTGCGTCAATGGGGCGGAGTT
GTTACGACATTTTGGAAAGTCCCGTTGATTTTGGTGCCAAAACAA
ACTCCCATTGACGTCAATGGGGTGGAGACTTGGAAATCCCCGTGA
GTCAAACCGCTATCCACGCCCATTGATGTACTGCCAAAACCGCAT
CACCATGGTAATAGCGATGACTAATACGTAGATGTACTGCCAAGT
AGGAAAGTCCCATAAGGTCATGTACTGGGCATAATGCCAGGCGGG
CCATTTACCGTCATTGACGTCAATAGGGGGCGTACTTGGCATATG
ATACACTTGATGTACTGCCAAGTGGGCAGTTTACCGTAAATACTC
CACCCATTGACGTCAATGGAAAGTCCCTATTGGCGTTACTATGGG
AACATACGTCATTATTGACGTCAATGGGCGGGGGTCGTTGGGCGG
TCAGCCAGGCGGGCCATTTACCGTAAGTTATGTAACGACGTCTCA
GCTGACAATGAGATCACATGGACACAGGAAGGGGAATATCACACT
CTGGGGACTGTGGTGGGGTCGGGGGAGGGGGGAGGGATAGCATTG
GGAGATATACCTAATGCTAGATGACACATTAGTGGGTGCAGCGCA
CCAGCATGGCACATGTATACATATGTAACTAACCTGCACAATGTG
CACATGTACCCTAAAACTTAGAGTATAATGGATCCGCAGGCCTCT
GCTAGCTTGACTGACTGAGATACAGCGTACCTTCAGCTCACAGAC
ATGATAAGATACATTGATGAGTTTGGACAAACCACAACTAGAATG
CAGTGAAAAAAATGCTTTATTTGTGAAATTTGTGATGCTATTGCT
TTATTTGTAACCATTATAAGCTGCAATAAACAAGTT
(SEQ ID NO: 36)
LINE-1 plasmid GFP (SEQ ID NO: 37)
CGGCCGCGGGGGGAGGAGCCAAGATGGCCGAATAGGAACAGCTCC
GGTCTACAGCTCCCAGCGTGAGCGACGCAGAAGACGGTGATTTCT
GCATTTCCATCTGAGGTACCGGGTTCATCTCACTAGGGAGTGCCA
GACAGTGGGCGCAGGCCAGTGTGTGTGCGCACCGTGCGCGAGCCG
AAGCAGGGCGAGGCATTGCCTCACCTGGGAAGCGCAAGGGGTCAG
GGAGTTCCCTTTCCGAGTCAAAGAAAGGGGTGACGGACGCACCTG
GAAAATCGGGTCACTCCCACCCGAATATTGCGCTTTTCAGACCGG
CTTAAGAAACGGCGCACCACGAGACTATATCCCACACCTGGCTCG
GAGGGTCCTACGCCCACGGAATCTCGCTGATTGCTAGCACAGCAG
TCTGAGATCAAACTGCAAGGCGGCAACGAGGCTGGGGGAGGGGCG
CCCGCCATTGCCCAGGCTTGCTTAGGTAAACAAAGCAGCAGGGAA
GCTCGAACTGGGTGGAGCCCACCACAGCTCAAGGAGGCCTGCCTG
CCTCTGTAGGCTCCACCTCTGGGGGCAGGGCACAGACAAACAAAA
AGACAGCAGTAACCTCTGCAGACTTAAGTGTCCCTGTCTGACAGC
TTTGAAGAGAGCAGTGGTTCTCCCAGCACGCAGCTGGAGATCTGA
GAACGGGCAGACTGCCTCCTCAAGTGGGTCCCTGACCCCTGACCC
CCGAGCAGCCTAACTGGGAGGCACCCCCCAGCAGGGGCACACTGA
CACCTCACACGGCAGGGTATTCCAACAGACCTGCAGCTGAGGGTC
CTGTCTGTTAGAAGGAAAACTAACAACCAGAAAGGACATCTACAC
CGAAAACCCATCTGTACATCACCATCATCAAAGACCAAAAGTAGA
TAAAACCACAAAGATGGGGAAAAAACAGAACAGAAAAACTGGAAA
CTCTAAAACGCAGAGCGCCTCTCCTCCTCCAAAGGAACGCAGTTC
CTCACCAGCAACAGAACAAAGCTGGATGGAGAATGATTTTGATGA
GCTGAGAGAAGAAGGCTTCAGACGATCAAATTACTCTGAGCTACG
GGAGGACATTCAAACCAAAGGCAAAGAAGTTGAAAACTTTGAAAA
AAATTTAGAAGAATGTATAACTAGAATAACCAATACAGAGAAGTG
CTTAAAGGAGCTGATGGAGCTGAAAACCAAGGCTCGAGAACTACG
TGAAGAATGCAGAAGCCTCAGGAGCCGATGCGATCAACTGGAAGA
AAGGGTATCAGCAATGGAAGATGAAATGAATGAAATGAAGCGAGA
AGGGAAGTTTAGAGAAAAAAGAATAAAAAGAAATGAGCAAAGCCT
CCAAGAAATATGGGACTATGTGAAAAGACCAAATCTACGTCTGAT
TGGTGTACCTGAAAGTGATGTGGAGAATGGAACCAAGTTGGAAAA
CACTCTGCAGGATATTATCCAGGAGAACTTCCCCAATCTAGCAAG
GCAGGCCAACGTTCAGATTCAGGAAATACAGAGAACGCCACAAAG
ATACTCCTCGAGAAGAGCAACTCCAAGACACATAATTGTCAGATT
CACCAAAGTTGAAATGAAGGAAAAAATGTTAAGGGCAGCCAGAGA
GAAAGGTCGGGTTACCCTCAAAGGAAAGCCCATCAGACTAACAGC
GGATCTCTCGGCAGAAACCCTACAAGCCAGAAGAGAGTGGGGGCC
AATATTCAACATTCTTAAAGAAAAGAATTTTCAACCCAGAATTTC
ATATCCAGCCAAACTAAGCTTCATAAGTGAAGGAGAAATAAAATA
CTTTATAGACAAGCAAATGTTGAGAGATTTTGTCACCACCAGGCC
TGCCCTAAAAGAGCTCCTGAAGGAAGCGCTAAACATGGAAAGGAA
CAACCGGTACCAGCCGCTGCAAAATCATGCCAAAATGTAAAGACC
ATCAAGACTAGGAAGAAACTGCATCAACTAATGAGCAAAATCACC
AGCTAACATCATAATGACAGGATCAACTTCACACATAACAATATT
AACTTTAAATATAAATGGACTAAATTCTGCAATTAAAAGACACAG
ACTGGCAAGTTGGATAAAGAGTCAAGACCCATCAGTGTGCTGTAT
TCAGGAAACCCATCTCACGTGCAGAGACACACATAGGCTCAAAAT
AAAAGGATGGAGGAAGATCTACCAAGCCAATGGAAAACAAAAAAA
GGCAGGGGTTGCAATCCTAGTCTCTGATAAAACAGACTTTAAACC
AACAAAGATCAAAAGAGACAAAGAAGGCCATTACATAATGGTAAA
GGGATCAATTCAACAAGAGGAGCTAACTATCCTAAATATTTATGC
ACCCAATACAGGAGCACCCAGATTCATAAAGCAAGTCCTCAGTGA
CCTACAAAGAGACTTAGACTCCCACACATTAATAATGGGAGACTT
TAACACCCCACTGTCAACATTAGACAGATCAACGAGACAGAAAGT
CAACAAGGATACCCAGGAATTGAACTCAGCTCTGCACCAAGCAGA
CCTAATAGACATCTACAGAACTCTCCACCCCAAATCAACAGAATA
TACATTTTTTTCAGCACCACACCACACCTATTCCAAAATTGACCA
CATAGTTGGAAGTAAAGCTCTCCTCAGCAAATGTAAAAGAACAGA
AATTATAACAAACTATCTCTCAGACCACAGTGCAATCAAACTAGA
ACTCAGGATTAAGAATCTCACTCAAAGCCGCTCAACTACATGGAA
ACTGAACAACCTGCTCCTGAATGACTACTGGGTACATAACGAAAT
GAAGGCAGAAATAAAGATGTTCTTTGAAACCAACGAGAACAAAGA
CACCACATACCAGAATCTCTGGGACGCATTCAAAGCAGTGTGTAG
AGGGAAATTTATAGCACTAAATGCCTACAAGAGAAAGCAGGAAAG
ATCCAAAATTGACACCCTAACATCACAATTAAAAGAACTAGAAAA
GCAAGAGCAAACACATTCAAAAGCTAGCAGAAGGCAAGAAATAAC
TAAAATCAGAGCAGAACTGAAGGAAATAGAGACACAAAAAACCCT
TCAAAAAATCAATGAATCCAGGAGCTGGTTTTTTGAAAGGATCAA
CAAAATTGATAGACCGCTAGCAAGACTAATAAAGAAAAAAAGAGA
GAAGAATCAAATAGACACAATAAAAAATGATAAAGGGGATATCAC
CACCGATCCCACAGAAATACAAACTACCATCAGAGAATACTACAA
ACACCTCTACGCAAATAAACTAGAAAATCTAGAAGAAATGGATAC
ATTCCTCGACACATACACTCTCCCAAGACTAAACCAGGAAGAAGT
TGAATCTCTGAATCGACCAATAACAGGCTCTGAAATTGTGGCAAT
AATCAATAGTTTACCAACCAAAAAGAGTCCAGGACCAGATGGATT
CACAGCCGAATTCTACCAGAGGTACAAGGAGGAACTGGTACCATT
CCTTCTGAAACTATTCCAATCAATAGAAAAAGAGGGAATCCTCCC
TAACTCATTTTATGAGGCCAGCATCATTCTGATACCAAAGCCGGG
CAGAGACACAACCAAAAAAGAGAATTTTAGACCAATATCCTTGAT
GAACATTGATGCAAAAATCCTCAATAAAATACTGGCAAACCGAAT
CCAGCAGCACATCAAAAAGCTTATCCACCATGATCAAGTGGGCTT
CATCCCTGGGATGCAAGGCTGGTTCAATATACGCAAATCAATAAA
TGTAATCCAGCATATAAACAGAGCCAAAGACAAAAACCACATGAT
TATCTCAATAGATGCAGAAAAAGCCTTTGACAAAATTCAACAACC
CTTCATGCTAAAAACTCTCAATAAATTAGGTATTGATGGGACGTA
TTTCAAAATAATAAGAGCTATCTATGACAAACCCACAGCCAATAT
CATACTGAATGGGCAAAAACTGGAAGCATTCCCTTTGAAAACCGG
CACAAGACAGGGATGCCCTCTCTCACCGCTCCTATTCAACATAGT
GTTGGAAGTTCTGGCCAGGGCAATCAGGCAGGAGAAGGAAATAAA
GGGTATTCAATTAGGAAAAGAGGAAGTCAAATTGTCCCTGTTTGC
AGACGACATGATTGTTTATCTAGAAAACCCCATCGTCTCAGCCCA
AAATCTCCTTAAGCTGATAAGCAACTTCAGCAAAGTCTCAGGATA
CAAAATCAATGTACAAAAATCACAAGCATTCTTATACACCAACAA
CAGACAAACAGAGAGCCAAATCATGGGTGAACTCCCATTCACAAT
TGCTTCAAAGAGAATAAAATACCTAGGAATCCAACTTACAAGGGA
TGTGAAGGACCTCTTCAAGGAGAACTACAAACCACTGCTCAAGGA
AATAAAAGAGGAGACAAACAAATGGAAGAACATTCCATGCTCATG
GGTAGGAAGAATCAATATCGTGAAAATGGCCATACTGCCCAAGGT
AATTTACAGATTCAATGCCATCCCCATCAAGCTACCAATGACTTT
CTTCACAGAATTGGAAAAAACTACTTTAAAGTTCATATGGAACCA
AAAAAGAGCCCGCATTGCCAAGTCAATCCTAAGCCAAAAGAACAA
AGCTGGAGGCATCACACTACCTGACTTCAAACTATACTACAAGGC
TACAGTAACCAAAACAGCATGGTACTGGTACCAAAACAGAGATAT
AGATCAATGGAACAGAACAGAGCCCTCAGAAATAATGCCGCATAT
CTACAACTATCTGATCTTTGACAAACCTGAGAAAAACAAGCAATG
GGGAAAGGATTCCCTATTTAATAAATGGTGCTGGGAAAACTGGCT
AGCCATATGTAGAAAGCTGAAACTGGATCCCTTCCTTACACCTTA
TACAAAAATCAATTCAAGATGGATTAAAGATTTAAACGTTAAACC
TAAAACCATAAAAACCCTAGAAGAAAACCTAGGCATTACCATTCA
GGACATAGGCGTGGGCAAGGACTTCATGTCCAAAACACCAAAAGC
AATGGCAACAAAAGACAAAATTGACAAATGGGATCTAATTAAACT
AAAGAGCTTCTGCACAGCAAAAGAAACTACCATCAGAGTGAACAG
GCAACCTACAACATGGGAGAAAATTTTTGCAACCTACTCATCTGA
CAAAGGGCTAATATCCAGAATCTACAATGAACTCAAACAAATTTA
CAAGAAAAAAACAAACAACCCCATCAAAAAGTGGGCGAAGGACAT
GAACAGACACTTCTCAAAAGAAGACATTTATGCAGCCAAAAAACA
CATGAAGAAATGCTCATCATCACTGGCCATCAGAGAAATGCAAAT
CAAAACCACTATGAGATATCATCTCACACCAGTTAGAATGGCAAT
CATTAAAAAGTCAGGAAACAACAGGTGCTGGAGAGGATGCGGAGA
AATAGGAACACTTTTACACTGTTGGTGGGACTGTAAACTAGTTCA
ACCATTGTGGAAGTCAGTGTGGCGATTCCTCAGGGATCTAGAACT
AGAAATACCATTTGACCCAGCCATCCCATTACTGGGTATATACCC
AAATGAGTATAAATCATGCTGCTATAAAGACACATGCACACGTAT
GTTTATTGCGGCACTATTCACAATAGCAAAGACTTGGAACCAACC
CAAATGTCCAACAATGATAGACTGGATTAAGAAAATGTGGCACAT
ATACACCATGGAATACTATGCAGCCATAAAAAATGATGAGTTCAT
ATCCTTTGTAGGGACATGGATGAAATTGGAAACCATCATTCTCAG
TAAACTATCGCAAGAACAAAAAACCAAACACCGCATATTCTCACT
CATAGGTGGGAATTGAACAATGAGATCACATGGACACAGGAAGGG
GAATATCACACTCTGGGGACTGTGGTGGGGTCGGGGGAGGGGGGA
GGGATAGCATTGGGAGATATACCTAATGCTAGATGACACATTAGT
GGGTGCAGCGCACCAGCATGGCACATGTATACGGATCCGAATTCT
CGACGGATCGATCCGAACAAACGACCCAACACCCGTGCGTTTTAT
TCTGTCTTTTTATTGCCGATCCCCTCAGAAGAACTCGTCAAGAAG
GCGATAGAAGGCGATGCGCTGCGAATCGGGAGCGGCGATACCGTA
AAGCACGAGGAAGCGGTCAGCCCATTCGCCGCCAAGCTCTTCAGC
AATATCACGGGTAGCCAACGCTATGTCCTGATAGCGGTCGGCCGC
TTTACTTGTACAGCTCGTCCATGCCGAGAGTGATCCCGGCGGCGG
TCACGAACTCCAGCAGGACCATGTGATCGCGCTTCTCGTTGGGGT
CTTTGCTCAGGGCGGACTGGGTGCTCAGGTAGTGGTTGTCGGGCA
GCAGCACGGGGCCGTCGCCGATGGGGGTGTTCTGCTGGTAGTGGT
CGGCCAGGTGAGTCCAGGAGATGTTTCAGCACTGTTGCCTTTAGT
CTCGAGGCAACTTAGACAACTGAGTATTGATCTGAGCACAGCAGG
GTGTGAGCTGTTTGAAGATACTGGGGTTGGGGGTGAAGAAACTGC
AGAGGACTAACTGGGCTGAGACCCAGTGGCAATGTTTTAGGGCCT
AAGGAATGCCTCTGAAAATCTAGATGGACAACTTTGACTTTGAGA
AAAGAGAGGTGGAAATGAGGAAAATGACTTTTCTTTATTAGATTT
CGGTAGAAAGAACTTTCATCTTTCCCCTATTTTTGTTATTCGTTT
TAAAACATCTATCTGGAGGCAGGACAAGTATGGTCATTAAAAAGA
TGCAGGCAGAAGGCATATATTGGCTCAGTCAAAGTGGGGAACTTT
GGTGGCCAAACATACATTGCTAAGGCTATTCCTATATCAGCTGGA
CACATATAAAATGCTGCTAATGCTTCATTACAAACTTATATCCTT
TAATTCCAGATGGGGGCAAAGTATGTCCAGGGGTGAGGAACAATT
GAAACATTTGGGCTGGAGTAGATTTTGAAAGTCAGCTCTGTGTGT
GTGTGTGTGTGTGTGTGTGTGAGAGCGTGTGTTTCTTTTAACGTT
TTCAGCCTACAGCATACAGGGTTCATGGTGGCAAGAAGATAACAA
GATTTAAATTATGGCCAGTGACTAGTGCTGCAAGAAGAACAACTA
CCTGCATTTAATGGGAAAGCAAAATCTCAGGCTTTGAGGGAAGTT
AACATAGGCTTGATTCTGGGTGGAAGCTGGGTGTGTAGTTATCTG
GAGGCCAGGCTGGAGCTCTCAGCTCACTATGGGTTCATCTTTATT
GTCTCCTTTCATCTCAACAGCTGCACGCTGCCGTCCTCGATGTTG
TGGCGGATCTTGAAGTTCACCTTGATGCCGTTCTTCTGCTTGTCG
GCCATGATATAGACGTTGTGGCTGTTGTAGTTGTACTCCAGCTTG
TGCCCCAGGATGTTGCCGTCCTCCTTGAAGTCGATGCCCTTCAGC
TCGATGCGGTTCACCAGGGTGTCGCCCTCGAACTTCACCTCGGCG
CGGGTCTTGTAGTTGCCGTCGTCCTTGAAGAAGATGGTGCGCTCC
TGGACGTAGCCTTCGGGCATGGCGGACTTGAAGAAGTCGTGCTGC
TTCATGTGGTCGGGGTAGCGGCTGAAGCACTGCACGCCGTAGGTC
AGGGTGGTCACGAGGGTGGGCCAGGGCACGGGCAGCTTGCCGGTG
GTGCAGATGAACTTCAGGGTCAGCTTGCCGTAGGTGGCATCGCCC
TCGCCCTCGCCGGACACGCTGAACTTGTGGCCGTTTACGTCGCCG
TCCAGCTCGACCAGGATGGGCACCACCCCGGTGAACAGCTCCTCG
CCCTTGCTCACCATGGTGGCGAATTCGAAGCTTGAGCTCGAGATC
TGAGTCCGGTAGCGCTAGCGGATCTGACGGTTCACTAAACCAGCT
CTGCTTATATAGACCTCCCACCGTACACGCCTACCGCCCATTTGC
GTCAATGGGGCGGAGTTGTTACGACATTTTGGAAAGTCCCGTTGA
TTTTGGTGCCAAAACAAACTCCCATTGACGTCAATGGGGTGGAGA
CTTGGAAATCCCCGTGAGTCAAACCGCTATCCACGCCCATTGATG
TACTGCCAAAACCGCATCACCATGGTAATAGCGATGACTAATACG
TAGATGTACTGCCAAGTAGGAAAGTCCCATAAGGTCATGTACTGG
GCATAATGCCAGGCGGGCCATTTACCGTCATTGACGTCAATAGGG
GGCGTACTTGGCATATGATACACTTGATGTACTGCCAAGTGGGCA
GTTTACCGTAAATACTCCACCCATTGACGTCAATGGAAAGTCCCT
ATTGGCGTTACTATGGGAACATACGTCATTATTGACGTCAATGGG
CGGGGGTCGTTGGGCGGTCAGCCAGGCGGGCCATTTACCGTAAGT
TATGTAACGCGGAACTCCATATATGGGCTATGAACTAATGACCCC
GTAATTGATTACTATTAGCCCGGGGGATCCAGACATGATAAGATA
CATTGATGAGTTTGGACAAACCACAACTAGAATGCAGTGAAAAAA
ATGCTTTATTTGTGAAATTTGTGATGCTATTGCTTTATTTGTAAC
CATTATAAGCTGCAATAAACAAGTTAACAACAACAATTGCATTCA
TTTTATGTTTCAGGTTCAGGGGGAGGTGTGGGAGGTTTTTTAAAG
CAAGTAAAACCTCTACAAATGTGGTATGGCTGATTATGATCCGGC
TGCCTCGCGCGTTTCGGTGATGACGGTGAAAACCTCTGACACATG
CAGCTCCCGGAGACGGTCACAGCTTGTCTGTAAGCGGATGCCGGG
AGCAGACAAGCCCGTCAGGGCGCGTCAGCGGGTGTTGGCGGGTGT
CGGGGCGCAGCCATGAGGTCGATCGACTCTAGAGGATCGATCCCC
GCCCCGGACGAACTAAACCTGACTACGACATCTCTGCCCCTTCTT
CGCGGGGCAGTGCATGTAATCCCTTCAGTTGGTTGGTACAACTTG
CCAACTGGGCCCTGTTCCACATGTGACACGGGGGGGGACCAAACA
CAAAGGGAGGCCTATATAAGCAGAGCTCGTTTAGTGAACCGTCAG
ATCGCCGGGGTTCTCTGACTGTAGTTGACATCCTTATAAATGGAT
GTGCACATTTGCCAACACTGAGTGGCTTTCATCCTGGAGCAGACT
TTGCAGTCTGTGGACTGCAACACAACATTGCCTTTATGTGTAACT
CTTGGCTGAAGCTCTTACACCAATGCTGGGGGACATGTACCTCCC
AGGGGCCCAGGAAGACTACGGGAGGCTACACCAACGTCAATCAGA
GGGGCCTGTGTAGCTACCGATAAGCGGACCCTCAAGAGGGCATTA
GCAATAGTGTTTATAAGGCCCCCTTGTTAACCCTAAACGGGTAGC
ATATGCTTCCCGGGTAGTAGTATATACTATCCAGACTAACCCTAA
TTCAATAGCATATGTTACCCAACGGGAAGCATATGCTATCGAATT
AGGGTTAGTAAAAGGGTCCTAAGGAACAGCGATATCTCCCACCCC
ATGAGCTGTCACGGTTTTATTTACATGGGGTCAGGATTCCACGAG
GGTAGTGAACCATTTTAGTCACAAGGGCAGTGGCTGAAGATCAAG
GAGCGGGCAGTGAACTCTCCTGAATCTTCGCCTGCTTCTTCATTC
TCCTTCGTTTAGCTAATAGAATAACTGCTGAGTTGTGAACAGTAA
GGTGTATGTGAGGTGCTCGAAAACAAGGTTTCAGGTGACGCCCCC
AGAATAAAATTTGGACGGGGGGTTCAGTGGTGGCATTGTGCTATG
ACACCAATATAACCCTCACAAACCCCTTGGGCAATAAATACTAGT
GTAGGAATGAAACATTCTGAATATCTTTAACAATAGAAATCCATG
GGGTGGGGACAAGCCGTAAAGACTGGATGTCCATCTCACACGAAT
TTATGGCTATGGGCAACACATAATCCTAGTGCAATATGATACTGG
GGTTATTAAGATGTGTCCCAGGCAGGGACCAAGACAGGTGAACCA
TGTTGTTACACTCTATTTGTAACAAGGGGAAAGAGAGTGGACGCC
GACAGCAGCGGACTCCACTGGTTGTCTCTAACACCCCCGAAAATT
AAACGGGGCTCCACGCCAATGGGGCCCATAAACAAAGACAAGTGG
CCACTCTTTTTTTTGAAATTGTGGAGTGGGGGCACGCGTCAGCCC
CCACACGCCGCCCTGCGGTTTTGGACTGTAAAATAAGGGTGTAAT
AACTTGGCTGATTGTAACCCCGCTAACCACTGCGGTCAAACCACT
TGCCCACAAAACCACTAATGGCACCCCGGGGAATACCTGCATAAG
TAGGTGGGCGGGCCAAGATAGGGGCGCGATTGCTGCGATCTGGAG
GACAAATTACACACACTTGCGCCTGAGCGCCAAGCACAGGGTTGT
TGGTCCTCATATTCACGAGGTCGCTGAGAGCACGGTGGGCTAATG
TTGCCATGGGTAGCATATACTACCCAAATATCTGGATAGCATATG
CTATCCTAATCTATATCTGGGTAGCATAGGCTATCCTAATCTATA
TCTGGGTAGCATATGCTATCCTAATCTATATCTGGGTAGTATATG
CTATCCTAATTTATATCTGGGTAGCATAGGCTATCCTAATCTATA
TCTGGGTAGCATATGCTATCCTAATCTATATCTGGGTAGTATATG
CTATCCTAATCTGTATCCGGGTAGCATATGCTATCCTAATAGAGA
TTAGGGTAGTATATGCTATCCTAATTTATATCTGGGTAGCATATA
CTACCCAAATATCTGGATAGCATATGCTATCCTAATCTATATCTG
GGTAGCATATGCTATCCTAATCTATATCTGGGTAGCATAGGCTAT
CCTAATCTATATCTGGGTAGCATATGCTATCCTAATCTATATCTG
GGTAGTATATGCTATCCTAATTTATATCTGGGTAGCATAGGCTAT
CCTAATCTATATCTGGGTAGCATATGCTATCCTAATCTATATCTG
GGTAGTATATGCTATCCTAATCTGTATCCGGGTAGCATATGCTAT
CCTCATGCATATACAGTCAGCATATGATACCCAGTAGTAGAGTGG
GAGTGCTATCCTTTGCATATGCCGCCACCTCCCAAGGGGGCGTGA
ATTTTCGCTGCTTGTCCTTTTCCTGCATGCTGGTTGCTCCCATTC
TTAGGTGAATTTAAGGAGGCCAGGCTAAAGCCGTCGCATGTCTGA
TTGCTCACCAGGTAAATGTCGCTAATGTTTTCCAACGCGAGAAGG
TGTTGAGCGCGGAGCTGAGTGACGTGACAACATGGGTATGCCCAA
TTGCCCCATGTTGGGAGGACGAAAATGGTGACAAGACAGATGGCC
AGAAATACACCAACAGCACGCATGATGTCTACTGGGGATTTATTC
TTTAGTGCGGGGGAATACACGGCTTTTAATACGATTGAGGGCGTC
TCCTAACAAGTTACATCACTCCTGCCCTTCCTCACCCTCATCTCC
ATCACCTCCTTCATCTCCGTCATCTCCGTCATCACCCTCCGCGGC
AGCCCCTTCCACCATAGGTGGAAACCAGGGAGGCAAATCTACTCC
ATCGTCAAAGCTGCACACAGTCACCCTGATATTGCAGGTAGGAGC
GGGCTTTGTCATAACAAGGTCCTTAATCGCATCCTTCAAAACCTC
AGCAAATATATGAGTTTGTAAAAAGACCATGAAATAACAGACAAT
GGACTCCCTTAGCGGGCCAGGTTGTGGGCCGGGTCCAGGGGCCAT
TCCAAAGGGGAGACGACTCAATGGTGTAAGACGACATTGTGGAAT
AGCAAGGGCAGTTCCTCGCCTTAGGTTGTAAAGGGAGGTCTTACT
ACCTCCATATACGAACACACCGGCGACCCAAGTTCCTTCGTCGGT
AGTCCTTTCTACGTGACTCCTAGCCAGGAGAGCTCTTAAACCTTC
TGCAATGTTCTCAAATTTCGGGTTGGAACCTCCTTGACCACGATG
CTTTCCAAACCACCCTCCTTTTTTGCGCCTGCCTCCATCACCCTG
ACCCCGGGGTCCAGTGCTTGGGCCTTCTCCTGGGTCATCTGCGGG
GCCCTGCTCTATCGCTCCCGGGGGCACGTCAGGCTCACCATCTGG
GCCACCTTCTTGGTGGTATTCAAAATAATCGGCTTCCCCTACAGG
GTGGAAAAATGGCCTTCTACCTGGAGGGGGCCTGCGCGGTGGAGA
CCCGGATGATGATGACTGACTACTGGGACTCCTGGGCCTCTTTTC
TCCACGTCCACGACCTCTCCCCCTGGCTCTTTCACGACTTCCCCC
CCTGGCTCTTTCACGTCCTCTACCCCGGCGGCCTCCACTACCTCC
TCGACCCCGGCCTCCACTACCTCCTCGACCCCGGCCTCCACTGCC
TCCTCGACCCCGGCCTCCACCTCCTGCTCCTGCCCCTCCTGCTCC
TGCCCCTCCTCCTGCTCCTGCCCCTCCTGCCCCTCCTGCTCCTGC
CCCTCCTGCCCCTCCTGCTCCTGCCCCTCCTGCCCCTCCTGCTCC
TGCCCCTCCTGCCCCTCCTCCTGCTCCTGCCCCTCCTGCCCCTCC
TCCTGCTCCTGCCCCTCCTGCCCCTCCTGCTCCTGCCCCTCCTGC
CCCTCCTGCTCCTGCCCCTCCTGCCCCTCCTGCTCCTGCCCCTCC
TGCTCCTGCCCCTCCTGCTCCTGCCCCTCCTGCTCCTGCCCCTCC
TGCCCCTCCTGCCCCTCCTCCTGCTCCTGCCCCTCCTGCTCCTGC
CCCTCCTGCCCCTCCTGCCCCTCCTGCTCCTGCCCCTCCTCCTGC
TCCTGCCCCTCCTGCCCCTCCTGCCCCTCCTCCTGCTCCTGCCCC
TCCTGCCCCTCCTCCTGCTCCTGCCCCTCCTCCTGCTCCTGCCCC
TCCTGCCCCTCCTGCCCCTCCTCCTGCTCCTGCCCCTCCTGCCCC
TCCTCCTGCTCCTGCCCCTCCTCCTGCTCCTGCCCCTCCTGCCCC
TCCTGCCCCTCCTCCTGCTCCTGCCCCTCCTCCTGCTCCTGCCCC
TCCTGCCCCTCCTGCCCCTCCTGCCCCTCCTCCTGCTCCTGCCCC
TCCTCCTGCTCCTGCCCCTCCTGCTCCTGCCCCTCCCGCTCCTGC
TCCTGCTCCTGTTCCACCGTGGGTCCCTTTGCAGCCAATGCAACT
TGGACGTTTTTGGGGTCTCCGGACACCATCTCTATGTCTTGGCCC
TGATCCTGAGCCGCCCGGGGCTCCTGGTCTTCCGCCTCCTCGTCC
TCGTCCTCTTCCCCGTCCTCGTCCATGGTTATCACCCCCTCTTCT
TTGAGGTCCACTGCCGCCGGAGCCTTCTGGTCCAGATGTGTCTCC
CTTCTCTCCTAGGCCATTTCCAGGTCCTGTACCTGGCCCCTCGTC
AGACATGATTCACACTAAAAGAGATCAATAGACATCTTTATTAGA
CGACGCTCAGTGAATACAGGGAGTGCAGACTCCTGCCCCCTCCAA
CAGCCCCCCCACCCTCATCCCCTTCATGGTCGCTGTCAGACAGAT
CCAGGTCTGAAAATTCCCCATCCTCCGAACCATCCTCGTCCTCAT
CACCAATTACTCGCAGCCCGGAAAACTCCCGCTGAACATCCTCAA
GATTTGCGTCCTGAGCCTCAAGCCAGGCCTCAAATTCCTCGTCCC
CCTTTTTGCTGGACGGTAGGGATGGGGATTCTCGGGACCCCTCCT
CTTCCTCTTCAAGGTCACCAGACAGAGATGCTACTGGGGCAACGG
AAGAAAAGCTGGGTGCGGCCTGTGAGGATCAGCTTATCGATGATA
AGCTGTCAAACATGAGAATTCTTGAAGACGAAAGGGCCTCGTGAT
ACGCCTATTTTTATAGGTTAATGTCATGATAATAATGGTTTCTTA
GACGTCAGGTGGCACTTTTCGGGGAAATGTGCGCGGAACCCCTAT
TTGTTTATTTTTCTAAATACATTCAAATATGTATCCGCTCATGAG
ACAATAACCCTGATAAATGCTTCAATAATATTGAAAAAGGAAGAG
TATGAGTATTCAACATTTCCGTGTCGCCCTTATTCCCTTTTTTGC
GGCATTTTGCCTTCCTGTTTTTGCTCACCCAGAAACGCTGGTGAA
AGTAAAAGATGCTGAAGATCAGTTGGGTGCACGAGTGGGTTACAT
CGAACTGGATCTCAACAGCGGTAAGATCCTTGAGAGTTTTCGCCC
CGAAGAACGTTTTCCAATGATGAGCACTTTTAAAGTTCTGCTATG
TGGCGCGGTATTATCCCGTGTTGACGCCGGGCAAGAGCAACTCGG
TCGCCGCATACACTATTCTCAGAATGACTTGGTTGAGTACTCACC
AGTCACAGAAAAGCATCTTACGGATGGCATGACAGTAAGAGAATT
ATGCAGTGCTGCCATAACCATGAGTGATAACACTGCGGCCAACTT
ACTTCTGACAACGATCGGAGGACCGAAGGAGCTAACCGCTTTTTT
GCACAACATGGGGGATCATGTAACTCGCCTTGATCGTTGGGAACC
GGAGCTGAATGAAGCCATACCAAACGACGAGCGTGACACCACGAT
GCCTGCAGCAATGGCAACAACGTTGCGCAAACTATTAACTGGCGA
ACTACTTACTCTAGCTTCCCGGCAACAATTAATAGACTGGATGGA
GGCGGATAAAGTTGCAGGACCACTTCTGCGCTCGGCCCTTCCGGC
TGGCTGGTTTATTGCTGATAAATCTGGAGCCGGTGAGCGTGGGTC
TCGCGGTATCATTGCAGCACTGGGGCCAGATGGTAAGCCCTCCCG
TATCGTAGTTATCTACACGACGGGGAGTCAGGCAACTATGGATGA
ACGAAATAGACAGATCGCTGAGATAGGTGCCTCACTGATTAAGCA
TTGGTAACTGTCAGACCAAGTTTACTCATATATACTTTAGATTGA
TTTAAAACTTCATTTTTAATTTAAAAGGATCTAGGTGAAGATCCT
TTTTGATAATCTCATGACCAAAATCCCTTAACGTGAGTTTTCGTT
CCACTGAGCGTCAGACCCCGTAGAAAAGATCAAAGGATCTTCTTG
AGATCCTTTTTTTCTGCGCGTAATCTGCTGCTTGCAAACAAAAAA
ACCACCGCTACCAGCGGTGGTTTGTTTGCCGGATCAAGAGCTACC
AACTCTTTTTCCGAAGGTAACTGGCTTCAGCAGAGCGCAGATACC
AAATACTGTCCTTCTAGTGTAGCCGTAGTTAGGCCACCACTTCAA
GAACTCTGTAGCACCGCCTACATACCTCGCTCTGCTAATCCTGTT
ACCAGTGGCTGCTGCCAGTGGCGATAAGTCGTGTCTTACCGGGTT
GGACTCAAGACGATAGTTACCGGATAAGGCGCAGCGGTCGGGCTG
AACGGGGGGTTCGTGCACACAGCCCAGCTTGGAGCGAACGACCTA
CACCGAACTGAGATACCTACAGCGTGAGCTATGAGAAAGCGCCAC
GCTTCCCGAAGGGAGAAAGGCGGACAGGTATCCGGTAAGCGGCAG
GGTCGGAACAGGAGAGCGCACGAGGGAGCTTCCAGGGGGAAACGC
CTGGTATCTTTATAGTCCTGTCGGGTTTCGCCACCTCTGACTTGA
GCGTCGATTTTTGTGATGCTCGTCAGGGGGGCGGAGCCTATGGAA
AAACGCCAGCAACGCGGCCTTTTTACGGTTCCTGGCCTTTTGCTG
GCCTTTTGCTCACATGTTCTTTCCTGCGTTATCCCCTGATTCTGT
GGATAACCGTATTACCGCCTTTGAGTGAGCTGATACCGCTCGCCG
CAGCCGAACGACCGAGCGCAGCGAGTCAGTGAGCGAGGAAGCGGA
AGAGCGCCTGATGCGGTATTTTCTCCTTACGCATCTGTGCGGTAT
TTCACACCGCATATGGTGCACTCTCAGTACAATCTGCTCTGATGC
CGCATAGTTAAGCCAGCTGTGGAATGTGTGTCAGTTAGGGTGTGG
AAAGTCCCCAGGCTCCCCAGCAGGCAGAAGTATGCAAAGCATGCA
TCTCAATTAGTCAGCAACCAGGTGTGGAAAGTCCCCAGGCTCCCC
AGCAGGCAGAAGTATGCAAAGCATGCATCTCAATTAGTCAGCAAC
CATAGTCCCGCCCCTAACTCCGCCCATCCCGCCCCTAACTCCGCC
CAGTTCCGCCCATTCTCCGCCCCATGGCTGACTAATTTTTTTTAT
TTATGCAGAGGCCGAGGCCGCCTCGGCCTCTGAGCTATTCCAGAA
GTAGTGAGGAGGCTTTTTTGGAGGCCTAGGCTTTTGCAAAAAGCT
TGCATGCCTGCAGGTCGGCCGCCACGACCGGTGCCGCCACCATCC
CCTGACCCACGCCCCTGACCCCTCACAAGGAGACGACCTTCCATG
ACCGAGTACAAGCCCACGGTGCGCCTCGCCACCCGCGACGACGTC
CCCCGGGCCGTACGCACCCTCGCCGCCGCGTTCGCCGACTACCCC
GCCACGCGCCACACCGTCGACCCGGACCGCCACATCGAGCGGGTC
ACCGAGCTGCAAGAACTCTTCCTCACGCGCGTCGGGCTCGACATC
GGCAAGGTGTGGGTCGCGGACGACGGCGCCGCGGTGGCGGTCTGG
ACCACGCCGGAGAGCGTCGAAGCGGGGGCGGTGTTCGCCGAGATC
GGCCCGCGCATGGCCGAGTTGAGCGGTTCCCGGCTGGCCGCGCAG
CAACAGATGGAAGGCCTCCTGGCGCCGCACCGGCCCAAGGAGCCC
GCGTGGTTCCTGGCCACCGTCGGCGTCTCGCCCGACCACCAGGGC
AAGGGTCTGGGCAGCGCCGTCGTGCTCCCCGGAGTGGAGGCGGCC
GAGCGCGCCGGGGTGCCCGCCTTCCTGGAGACCTCCGCGCCCCGC
AACCTCCCCTTCTACGAGCGGCTCGGCTTCACCGTCACCGCCGAC
GTCGAGGTGCCCGAAGGACCGCGCACCTGGTGCATGACCCGCAAG
CCCGGTGCCTGACGCCCGCCCCACGACCCGCAGCGCCCGACCGAA
AGGAGCGCACGACCCCATGGCTCCGACCGAAGCCGACCCGGGCGG
CCCCGCCGACCCCGCACCCGCCCCCGAGGCCCACCGACTCTAGAG
GATCATAATCAGCCATACCACATTTGTAGAGGTTTTACTTGCTTT
AAAAAACCTCCCACACCTCCCCCTGAACCTGAAACATAAAATGAA
TGCAATTGTTGTTGTTAACTTGTTTATTGCAGCTTATAATGGTTA
CAAATAAAGCAATAGCATCACAAATTTCACAAATAAAGCATTTTT
TTCACTGCATTCTAGTTGTGGTTTGTCCAAACTCATCAATGTATC
TTATCATGTCTGGATCACTCGCCGATAGTGGAAACCGACGCCCCA
GCACTCGTCCGAGGGCAAAGGAATAGGGGAGATGGGGGAGGCTAA
CTGAAACACGGAAGGAGACAATACCGGAAGGAACCCGCGCTATGA
CGGCAATAAAAAGACAGAATAAAACGCACGGGTGTTGGGTCGTTT
GTTCATAAACGCGGGGTTCGGTCCCAGGGCTGGCACTCTGTCGAT
ACCCCACCGAGACCCCATTGGGGCCAATACGCCCGCGTTTCTTCC
TTTTCCCCACCCCACCCCCCAAGTTCGGGTGAAGGCCCAGGGCTC
GCAGCCAACGTCGGGGCGGCAGGCCCTGCCATAGCCACTGGCCCC
GTGGGTTAGGGACGGGGTCCCCCATGGGGAATGGTTTATGGTTCG
TGGGGGTTATTATTTTGGGCGTTGCGTGGGGTCTGGTCCACGACT
GGACTGAGCAGACAGACCCATGGTTTTTGGATGGCCTGGGCATGG
ACCGCATGTACTGGCGCGACACGAACACCGGGCGTCTGTGGCTGC
CAAACACCCCCGACCCCCAAAAACCACCGCGCGGATTTCTGGCGT
GCCAAGCTAGTCGACCAATTCTCATGTTTGACAGCTTATCATCGC
AGATCCGGGCAACGTTGTTGCATTGCTGCAGGCGCAGAACTGGTA
GGTATGGAAGATCTCTAGAAGCTGGGTACCAGCTGCTAGCAAGCT
TGCTAGCGGCCGGCTCGAGTTTACTCCCTATCAGTGATAGAGAAC
GTATGTCGAGTTTACTCCCTATCAGTGATAGAGAACGATGTCGAG
TTTACTCCCTATCAGTGATAGAGAACGTATGTCGAGTTTACTCCC
TATCAGTGATAGAGAACGTATGTCGAGTTTACTCCCTATCAGTGA
TAGAGAACGTATGTCGAGTTTATCCCTATCAGTGATAGAGAACGT
ATGTCGAGTTTACTCCCTATCAGTGATAGAGAACGTATGTCGAGG
TAGGCGTGTACGGTG (SEQ ID NO: 37)
LINE1-GFP mRNA (SEQ ID NO: 38)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGTGAGACAGCCGTCAGACCATCAAGACTAGGA
AGAAACTGCATCAACTAATGAGCAAAATCACCAGCTAACATCATA
GTATACATGACCGGCTCTAACTCACATATCACCATCCTTACACTT
AACATTAACGGCCTCAACTCAGCTATCAAGCGCCATCGGCTGGCC
AGCTGGATCAAATCACAGGATCCAAGCGTTTGTTGCATCCAAGAG
ACCCACCTGACCTGTAGAGATACTCACCGCCTCAAGATCAAGGGA
TGGCGAAAGATTTATCAGGCGAACGGTAAGCAGAAGAAAGCCGGA
GTCGCAATTCTGGTCTCAGACAAGACGGATTTCAAGCCCACCAAA
ATTAAGCGTGATAAGGAAGGTCACTATATTATGGTGAAAGGCAGC
ATACAGCAGGAAGAACTTACCATATTGAACATCTACGCGCCAAAC
ACCGGCGCACCTCGCTTTATCAAACAGGTCCTGTCCGATCTGCAG
CGAGATCTGGATTCTCATACGTTGATTATGGGTGATTTCAATACA
CCATTGAGCACCCTGGATCGCAGCACCAGGCAAAAGGTAAATAAA
GACACGCAAGAGCTCAATAGCGCACTGCATCAGGCAGATCTCATT
GATATTTATCGCACTCTTCATCCTAAGAGTACCGAGTACACATTC
TTCAGCGCCCCACATCATACATACTCAAAGATCGATCATATCGTC
GGCTCAAAGGCTCTGCTGTCAAAGTGCAAGCGCACAGAGATAATT
ACAAATTACCTGTCAGATCATAGCGCGATCAAGCTCGAGCTGAGA
ATCAAGAACCTGACCCAGAGCCGGAGTACCACTTGGAAGCTTAAT
AACCTGCTGCTCAACGATTATTGGGTCCACAATGAGATGAAGGCA
GAGATTAAAATGTTCTTCGAAACAAATGAGAATAAGGATACTACC
TATCAAAACCTTTGGGATGCCTTTAAGGCCGTCTGCAGAGGCAAG
TTCATCGCCCTCAACGCCTATAAAAGAAAACAAGAGAGATCTAAG
ATCGATACTCTCACCTCTCAGCTGAAGGAGTTGGAGAAACAGGAA
CAGACCCACTCCAAGGCGTCAAGACGGCAGGAGATCACAAAGATT
CGCGCCGAGTTGAAAGAGATCGAAACCCAAAAGACTCTTCAGAAA
ATTAACGAGTCTCGTAGTTGGTTCTTCGAGCGGATTAATAAGATA
GACAGACCTCTGGCACGACTGATTAAGAAGAAGCGCGAAAAGAAC
CAGATTGATACCATCAAGAACGACAAGGGCGACATCACTACTGAC
CCGACCGAGATCCAGACCACTATTCGGGAGTATTATAAGCATTTG
TATGCTAACAAGCTTGAGAACCTGGAAGAGATGGACACTTTTCTG
GATACCTATACTCTGCCACGGCTTAATCAAGAGGAAGTCGAGTCC
CTCAACCGCCCAATTACAGGAAGCGAGATTGTGGCCATAATTAAC
TCCCTGCCGACAAAGAAATCTCCTGGTCCGGACGGGTTTACAGCT
GAGTTTTATCAACGGTATATGGAAGAGCTTGTACCGTTTCTGCTC
AAGCTCTTTCAGTCTATAGAAAAGGAAGGCATCTTGCCCAATTCC
TTCTACGAAGCTTCTATAATACTTATTCCCAAACCAGGACGCGAT
ACCACAAAGAAGGAAAACTTCCGGCCCATTAGTCTCATGAATATC
GACGCTAAAATATTGAACAAGATTCTCGCCAACAGAATCCAACAA
CATATTAAGAAATTGATACATCACGACCAGGTGGGGTTTATACCT
GGCATGCAGGGCTGGTTTAACATCCGGAAGAGTATTAACGTCATT
CAACACATTAATAGAGCTAAGGATAAGAATCATATGATCATCTCT
ATAGACGCGGAAAAGGCATTCGATAAGATTCAGCAGCCATTTATG
CTCAAGACTCTGAACAAACTCGGCATCGACGGAACATATTTTAAG
ATTATTCGCGCAATTTACGATAAGCCGACTGCTAACATTATCCTT
AACGGCCAAAAGCTCGAGGCCTTTCCGCTCAAGACTGGAACCCGC
CAAGGCTGTCCCCTCTCCCCGCTTTTGTTTAATATTGTACTCGAG
GTGCTGGCTAGGGCTATTCGTCAAGAGAAAGAGATTAAAGGGATA
CAGCTCGGGAAGGAAGAGGTCAAGCTTTCCTTGTTCGCCGATGAT
ATGATTGTGTACCTGGAGAATCCTATTGTGTCTGCTCAGAACCTT
CTTAAACTTATTTCTAACTTTAGCAAGGTCAGCGGCTATAAGATT
AACGTCCAGAAATCTCAGGCCTTTCTGTACACAAATAATCGACAG
ACCGAATCCCAGATAATGGGTGAGCTTCCGTTTGTCATAGCCAGC
AAAAGGATAAAGTATCTCGGAATCCAGCTGACACGAGACGTTAAA
GATTTGTTTAAGGAAAATTACAAGCCTCTCCTGAAAGAGATTAAG
GAAGATACTAATAAGTGGAAGAATATCCCCTGTTCATGGGTTGGC
AGAATCAACATAGTGAAGATGGCAATACTTCCTAAAGTGATATAT
CGCTTTAACGCCATCCCAATTAAACTGCCTATGACCTTCTTTACG
GAGCTCGAGAAAACAACCCTTAAATTTATATGGAATCAAAAGAGA
GCAAGAATAGCGAAGTCCATCTTGAGCCAGAAGAATAAGGCCGGT
GGGATTACTTTGCCTGATTTTAAGTTGTATTATAAAGCCACAGTA
ACTAAGACAGCCTGGTATTGGTATCAGAATAGAGACATCGACCAG
TGGAATCGGACCGAACCATCAGAGATAATGCCCCACATCTATAAT
TACCTTATATTCGATAAGCCAGAAAAGAATAAACAGTGGGGCAAA
GACAGCCTCTTCAACAAGTGGTGTTGGGAGAATTGGCTGGCCATA
TGCCGGAAACTCAAGCTCGACCCCTTTCTTACACCCTACACTAAA
ATCAACAGTAGGTGGATCAAGGACTTGAATGTCAAGCCAAAGACT
ATAAAGACACTGGAAGAGAATCTTGGGATCACAATACAAGATATA
GGCGTCGGCAAAGATTTTATGTCAAAGACGCCCAAGGCCATGGCC
ACTAAGGATAAGATTGATAAGTGGGACCTTATTAAGCTCAAAAGC
TTCTGTACTGCCAAGGAGACCACGATCAGAGTTAATAGGCAGCCC
ACTACATGGGAAAAGATTTTCGCCACTTATTCATCAGATAAGGGG
TTGATAAGCAGAATATATAACGAGCTGAAGCAGATCTACAAGAAG
AAAACGAATAATCCCATCAAGAAGTGGGCAAAAGATATGAACAGG
CATTTTAGCAAAGAGGATATCTACGCCGCGAAGAAGCATATGAAG
AAGTGTAGTTCAAGCTTGGCCATTCGTGAGATGCAGATTAAGACG
ACCATGCGATACCACCTTACCCCAGTGAGGATGGCAATTATCAAG
AAATCTGGCAATAATAGATGTTGGCGGGGCTGTGGCGAGATTGGC
ACCCTGCTCCATTGCTGGTGGGATTGCAAGCTGGTGCAGCCGCTT
TGGAAATCAGTCTGGCGCTTTCTGAGGGACCTCGAGCTTGAGATT
CCCTTCGATCCCGCAATTCCCTTGCTCGGAATCTATCCTAACGAA
TACAAGAGCTGTTGTTACAAGGATACGTGTACCCGGATGTTCATC
GCGGCCTTGTTTACGATAGCTAAGACGTGGAATCAGCCTAAGTGC
CCCACAATGATCGATTGGATCAAGAAAATGTGGCATATTTATACC
ATGGAGTATTACGCAGCAATTAAGAATGACGAATTTATTTCCTTC
GTTGGGACCTGGATGAAGCTGGAGACTATTATTCTGAGCAAGCTG
TCTCAGGAGCAAAAGACAAAGCATAGAATCTTCTCTCTCATTGGT
GGTAACGACTACAAAGACGATGACGACAAGTAAAGCGCTTCTAGA
AGTTGTCTCCTCCTGCACTGACTGACTGATACAATCGATTTCTGG
ATCCGCAGGCCTAATCAACCTCTGGATTACAAAATTTGTGAAAGA
TTGACTGGTATTCTTAACTATGTTGCTCCTTTTACGCTATGTGGA
TACGCTGCTTTAATGCCTTTGTATCATGCTATTGCTTCCCGTATG
GCTTTCATTTTCTCCTCCTTGTATAAATCCTGGTTGCTGTCTCTT
TATGAGGAGTTGTGGCCCGTTGTCAGGCAACGTGGCGTGGTGTGC
ACTGTGTTTGCTGACGCAACCCCCACTGGTTGGGGCATTGCCACC
ACCTGTCAGCTCCTTTCCGGGACTTTCGCTTTCCCCCTCCCTATT
GCCACGGCGGAACTCATCGCCGCCTGCCTTGCCCGCTGCTGGACA
GGGGCTCGGCTGTTGGGCACTGACAATTCCGTGGTGTTGTCGGGG
AAGCTGACGTCCTTTCCATGGCTGCTCGCCTGTGTTGCCACCTGG
ATTCTGCGCGGGACGTCCTTCTGCTACGTCCCTTCGGCCCTCAAT
CCAGCGGACCTTCCTTCCCGCTGAGAGACACAAAAAATTCCAACA
CACTATTGCAATGAAAATAAATTTCCTTTATTAGCCAGAAGTCAG
ATGCTCAAGGGGCTTCATGATGTCCCCATAATTTTTGGCAGAGGG
AAAAAGATCTCAGTGGTATTTGTGAGCCAGGGCATTGGCCTTCTG
ATAGGCAGCCTGCACCTGAGGAGTGCGGCCGCTTTACTTGTACAG
CTCGTCCATGCCGAGAGTGATCCCGGCGGCGGTCACGAACTCCAG
CAGGACCATGTGATCGCGCTTCTCGTTGGGGTCTTTGCTCAGGGC
GGACTGGGTGCTCAGGTAGTGGTTGTCGGGCAGCAGCACGGGGCC
GTCGCCGATGGGGGTGTTCTGCTGGTAGTGGTCGGCGAGCTGCAC
GCTGCCGTCCTCGATGTTGTGGCGGATCTTGAAGTTCACCTTGAT
GCCGTTCTTCTGCTTGTCGGCCATGATATAGACGTTGTGGCTGTT
GTAGTTGTACTCCAGCTTGTGCCCCAGGATGTTGCCGTCCTCCTT
GAAGTCGATGCCCTTCAGCTCGATGCGGTTCACCAGGGTGTCGCC
CTCGAACTTCACCTCGGCGCGGGTCTTGTAGTTGCCGTCGTCCTT
GAAGAAGATGGTGCGCTCCTGGACGTAGCCTTCGGGCATGGCGGA
CTTGAAGAAGTCGTGCTGCTTCATGTGGTCGGGGTAGCGGCTGAA
GCACTGCACGCCGTAGGTCAGGGTGGTCACGAGGGTGGGCCAGGG
CACGGGCAGCTTGCCGGTGGTGCAGATGAACTTCAGGGTCAGCTT
GCCGTAGGTGGCATCGCCCTCGCCCTCGCCGGACACGCTGAACTT
GTGGCCGTTTACGTCGCCGTCCAGCTCGACCAGGATGGGCACCAC
CCCGGTGAACAGCTCCTCGCCCTTGCTCACCATGGTGGCGGGATC
TGACGGTTCACTAAACCAGCTCTGCTTATATAGACCTCCCACCGT
ACACGCCTACCGCCCATTTGCGTCAATGGGGCGGAGTTGTTACGA
CATTTTGGAAAGTCCCGTTGATTTTGGTGCCAAAACAAACTCCCA
TTGACGTCAATGGGGTGGAGACTTGGAAATCCCCGTGAGTCAAAC
CGCTATCCACGCCCATTGATGTACTGCCAAAACCGCATCACCATG
GTAATAGCGATGACTAATACGTAGATGTACTGCCAAGTAGGAAAG
TCCCATAAGGTCATGTACTGGGCATAATGCCAGGCGGGCCATTTA
CCGTCATTGACGTCAATAGGGGGCGTACTTGGCATATGATACACT
TGATGTACTGCCAAGTGGGCAGTTTACCGTAAATACTCCACCCAT
TGACGTCAATGGAAAGTCCCTATTGGCGTTACTATGGGAACATAC
GTCATTATTGACGTCAATGGGCGGGGGTCGTTGGGCGGTCAGCCA
GGCGGGCCATTTACCGTAAGTTATGTAACGGGCCTGCTGCCGGCT
CTGCGGCCTCTTCCGCGTCTTCGCCTTCGCCCTCAGACGAGTCGG
ATCTCCCTTTGGGCCGCCTCCCCGCCTGTCTAGCTTGACTGACTG
AGATACAGCGTACCTTCAGCTCACAGACATGATAAGATACATTGA
TGAGTTTGGACAAACCACAACTAGAATGCAGTGAAAAAAATGCTT
TATTTGTGAAATTTGTGATGCTATTGCTTTATTTGTAACCATTAT
AAGCTGCAATAAACAAGTT
(SEQ ID NO: 38)
LINE-1-plasmid_CD5-intron-FCR-PI3K
(SEQ ID NO: 39)
CGGCCGCGGGGGGAGGAGCCAAGATGGCCGAATAGGAACAGCTCC
GGTCTACAGCTCCCAGCGTGAGCGACGCAGAAGACGGTGATTTCT
GCATTTCCATCTGAGGTACCGGGTTCATCTCACTAGGGAGTGCCA
GACAGTGGGCGCAGGCCAGTGTGTGTGCGCACCGTGCGCGAGCCG
AAGCAGGGCGAGGCATTGCCTCACCTGGGAAGCGCAAGGGGTCAG
GGAGTTCCCTTTCCGAGTCAAAGAAAGGGGTGACGGACGCACCTG
GAAAATCGGGTCACTCCCACCCGAATATTGCGCTTTTCAGACCGG
CTTAAGAAACGGCGCACCACGAGACTATATCCCACACCTGGCTCG
GAGGGTCCTACGCCCACGGAATCTCGCTGATTGCTAGCACAGCAG
TCTGAGATCAAACTGCAAGGCGGCAACGAGGCTGGGGGAGGGGCG
CCCGCCATTGCCCAGGCTTGCTTAGGTAAACAAAGCAGCAGGGAA
GCTCGAACTGGGTGGAGCCCACCACAGCTCAAGGAGGCCTGCCTG
CCTCTGTAGGCTCCACCTCTGGGGGCAGGGCACAGACAAACAAAA
AGACAGCAGTAACCTCTGCAGACTTAAGTGTCCCTGTCTGACAGC
TTTGAAGAGAGCAGTGGTTCTCCCAGCACGCAGCTGGAGATCTGA
GAACGGGCAGACTGCCTCCTCAAGTGGGTCCCTGACCCCTGACCC
CCGAGCAGCCTAACTGGGAGGCACCCCCCAGCAGGGGCACACTGA
CACCTCACACGGCAGGGTATTCCAACAGACCTGCAGCTGAGGGTC
CTGTCTGTTAGAAGGAAAACTAACAACCAGAAAGGACATCTACAC
CGAAAACCCATCTGTACATCACCATCATCAAAGACCAAAAGTAGA
TAAAACCACAAAGATGGGGAAAAAACAGAACAGAAAAACTGGAAA
CTCTAAAACGCAGAGCGCCTCTCCTCCTCCAAAGGAACGCAGTTC
CTCACCAGCAACAGAACAAAGCTGGATGGAGAATGATTTTGATGA
GCTGAGAGAAGAAGGCTTCAGACGATCAAATTACTCTGAGCTACG
GGAGGACATTCAAACCAAAGGCAAAGAAGTTGAAAACTTTGAAAA
AAATTTAGAAGAATGTATAACTAGAATAACCAATACAGAGAAGTG
CTTAAAGGAGCTGATGGAGCTGAAAACCAAGGCTCGAGAACTACG
TGAAGAATGCAGAAGCCTCAGGAGCCGATGCGATCAACTGGAAGA
AAGGGTATCAGCAATGGAAGATGAAATGAATGAAATGAAGCGAGA
AGGGAAGTTTAGAGAAAAAAGAATAAAAAGAAATGAGCAAAGCCT
CCAAGAAATATGGGACTATGTGAAAAGACCAAATCTACGTCTGAT
TGGTGTACCTGAAAGTGATGTGGAGAATGGAACCAAGTTGGAAAA
CACTCTGCAGGATATTATCCAGGAGAACTTCCCCAATCTAGCAAG
GCAGGCCAACGTTCAGATTCAGGAAATACAGAGAACGCCACAAAG
ATACTCCTCGAGAAGAGCAACTCCAAGACACATAATTGTCAGATT
CACCAAAGTTGAAATGAAGGAAAAAATGTTAAGGGCAGCCAGAGA
GAAAGGTCGGGTTACCCTCAAAGGAAAGCCCATCAGACTAACAGC
GGATCTCTCGGCAGAAACCCTACAAGCCAGAAGAGAGTGGGGGCC
AATATTCAACATTCTTAAAGAAAAGAATTTTCAACCCAGAATTTC
ATATCCAGCCAAACTAAGCTTCATAAGTGAAGGAGAAATAAAATA
CTTTATAGACAAGCAAATGTTGAGAGATTTTGTCACCACCAGGCC
TGCCCTAAAAGAGCTCCTGAAGGAAGCGCTAAACATGGAAAGGAA
CAACCGGTACCAGCCGCTGCAAAATCATGCCAAAATGTAAAGACC
ATCAAGACTAGGAAGAAACTGCATCAACTAATGAGCAAAATCACC
AGCTAACATCATAATGACAGGATCAACTTCACACATAACAATATT
AACTTTAAATATAAATGGACTAAATTCTGCAATTAAAAGACACAG
ACTGGCAAGTTGGATAAAGAGTCAAGACCCATCAGTGTGCTGTAT
TCAGGAAACCCATCTCACGTGCAGAGACACACATAGGCTCAAAAT
AAAAGGATGGAGGAAGATCTACCAAGCCAATGGAAAACAAAAAAA
GGCAGGGGTTGCAATCCTAGTCTCTGATAAAACAGACTTTAAACC
AACAAAGATCAAAAGAGACAAAGAAGGCCATTACATAATGGTAAA
GGGATCAATTCAACAAGAGGAGCTAACTATCCTAAATATTTATGC
ACCCAATACAGGAGCACCCAGATTCATAAAGCAAGTCCTCAGTGA
CCTACAAAGAGACTTAGACTCCCACACATTAATAATGGGAGACTT
TAACACCCCACTGTCAACATTAGACAGATCAACGAGACAGAAAGT
CAACAAGGATACCCAGGAATTGAACTCAGCTCTGCACCAAGCAGA
CCTAATAGACATCTACAGAACTCTCCACCCCAAATCAACAGAATA
TACATTTTTTTCAGCACCACACCACACCTATTCCAAAATTGACCA
CATAGTTGGAAGTAAAGCTCTCCTCAGCAAATGTAAAAGAACAGA
AATTATAACAAACTATCTCTCAGACCACAGTGCAATCAAACTAGA
ACTCAGGATTAAGAATCTCACTCAAAGCCGCTCAACTACATGGAA
ACTGAACAACCTGCTCCTGAATGACTACTGGGTACATAACGAAAT
GAAGGCAGAAATAAAGATGTTCTTTGAAACCAACGAGAACAAAGA
CACCACATACCAGAATCTCTGGGACGCATTCAAAGCAGTGTGTAG
AGGGAAATTTATAGCACTAAATGCCTACAAGAGAAAGCAGGAAAG
ATCCAAAATTGACACCCTAACATCACAATTAAAAGAACTAGAAAA
GCAAGAGCAAACACATTCAAAAGCTAGCAGAAGGCAAGAAATAAC
TAAAATCAGAGCAGAACTGAAGGAAATAGAGACACAAAAAACCCT
TCAAAAAATCAATGAATCCAGGAGCTGGTTTTTTGAAAGGATCAA
CAAAATTGATAGACCGCTAGCAAGACTAATAAAGAAAAAAAGAGA
GAAGAATCAAATAGACACAATAAAAAATGATAAAGGGGATATCAC
CACCGATCCCACAGAAATACAAACTACCATCAGAGAATACTACAA
ACACCTCTACGCAAATAAACTAGAAAATCTAGAAGAAATGGATAC
ATTCCTCGACACATACACTCTCCCAAGACTAAACCAGGAAGAAGT
TGAATCTCTGAATCGACCAATAACAGGCTCTGAAATTGTGGCAAT
AATCAATAGTTTACCAACCAAAAAGAGTCCAGGACCAGATGGATT
CACAGCCGAATTCTACCAGAGGTACAAGGAGGAACTGGTACCATT
CCTTCTGAAACTATTCCAATCAATAGAAAAAGAGGGAATCCTCCC
TAACTCATTTTATGAGGCCAGCATCATTCTGATACCAAAGCCGGG
CAGAGACACAACCAAAAAAGAGAATTTTAGACCAATATCCTTGAT
GAACATTGATGCAAAAATCCTCAATAAAATACTGGCAAACCGAAT
CCAGCAGCACATCAAAAAGCTTATCCACCATGATCAAGTGGGCTT
CATCCCTGGGATGCAAGGCTGGTTCAATATACGCAAATCAATAAA
TGTAATCCAGCATATAAACAGAGCCAAAGACAAAAACCACATGAT
TATCTCAATAGATGCAGAAAAAGCCTTTGACAAAATTCAACAACC
CTTCATGCTAAAAACTCTCAATAAATTAGGTATTGATGGGACGTA
TTTCAAAATAATAAGAGCTATCTATGACAAACCCACAGCCAATAT
CATACTGAATGGGCAAAAACTGGAAGCATTCCCTTTGAAAACCGG
CACAAGACAGGGATGCCCTCTCTCACCGCTCCTATTCAACATAGT
GTTGGAAGTTCTGGCCAGGGCAATCAGGCAGGAGAAGGAAATAAA
GGGTATTCAATTAGGAAAAGAGGAAGTCAAATTGTCCCTGTTTGC
AGACGACATGATTGTTTATCTAGAAAACCCCATCGTCTCAGCCCA
AAATCTCCTTAAGCTGATAAGCAACTTCAGCAAAGTCTCAGGATA
CAAAATCAATGTACAAAAATCACAAGCATTCTTATACACCAACAA
CAGACAAACAGAGAGCCAAATCATGGGTGAACTCCCATTCACAAT
TGCTTCAAAGAGAATAAAATACCTAGGAATCCAACTTACAAGGGA
TGTGAAGGACCTCTTCAAGGAGAACTACAAACCACTGCTCAAGGA
AATAAAAGAGGAGACAAACAAATGGAAGAACATTCCATGCTCATG
GGTAGGAAGAATCAATATCGTGAAAATGGCCATACTGCCCAAGGT
AATTTACAGATTCAATGCCATCCCCATCAAGCTACCAATGACTTT
CTTCACAGAATTGGAAAAAACTACTTTAAAGTTCATATGGAACCA
AAAAAGAGCCCGCATTGCCAAGTCAATCCTAAGCCAAAAGAACAA
AGCTGGAGGCATCACACTACCTGACTTCAAACTATACTACAAGGC
TACAGTAACCAAAACAGCATGGTACTGGTACCAAAACAGAGATAT
AGATCAATGGAACAGAACAGAGCCCTCAGAAATAATGCCGCATAT
CTACAACTATCTGATCTTTGACAAACCTGAGAAAAACAAGCAATG
GGGAAAGGATTCCCTATTTAATAAATGGTGCTGGGAAAACTGGCT
AGCCATATGTAGAAAGCTGAAACTGGATCCCTTCCTTACACCTTA
TACAAAAATCAATTCAAGATGGATTAAAGATTTAAACGTTAAACC
TAAAACCATAAAAACCCTAGAAGAAAACCTAGGCATTACCATTCA
GGACATAGGCGTGGGCAAGGACTTCATGTCCAAAACACCAAAAGC
AATGGCAACAAAAGACAAAATTGACAAATGGGATCTAATTAAACT
AAAGAGCTTCTGCACAGCAAAAGAAACTACCATCAGAGTGAACAG
GCAACCTACAACATGGGAGAAAATTTTTGCAACCTACTCATCTGA
CAAAGGGCTAATATCCAGAATCTACAATGAACTCAAACAAATTTA
CAAGAAAAAAACAAACAACCCCATCAAAAAGTGGGCGAAGGACAT
GAACAGACACTTCTCAAAAGAAGACATTTATGCAGCCAAAAAACA
CATGAAGAAATGCTCATCATCACTGGCCATCAGAGAAATGCAAAT
CAAAACCACTATGAGATATCATCTCACACCAGTTAGAATGGCAAT
CATTAAAAAGTCAGGAAACAACAGGTGCTGGAGAGGATGCGGAGA
AATAGGAACACTTTTACACTGTTGGTGGGACTGTAAACTAGTTCA
ACCATTGTGGAAGTCAGTGTGGCGATTCCTCAGGGATCTAGAACT
AGAAATACCATTTGACCCAGCCATCCCATTACTGGGTATATACCC
AAATGAGTATAAATCATGCTGCTATAAAGACACATGCACACGTAT
GTTTATTGCGGCACTATTCACAATAGCAAAGACTTGGAACCAACC
CAAATGTCCAACAATGATAGACTGGATTAAGAAAATGTGGCACAT
ATACACCATGGAATACTATGCAGCCATAAAAAATGATGAGTTCAT
ATCCTTTGTAGGGACATGGATGAAATTGGAAACCATCATTCTCAG
TAAACTATCGCAAGAACAAAAAACCAAACACCGCATATTCTCACT
CATAGGTGGGAATTGAACAATGAGATCACATGGACACAGGAAGGG
GAATATCACACTCTGGGGACTGTGGTGGGGTCGGGGGAGGGGGGA
GGGATAGCATTGGGAGATATACCTAATGCTAGATGACACATTAGT
GGGTGCAGCGCACCAGCATGGCACATGTATACGGATCCGAATTCT
CGACGGATCGATCCGAACAAACGACCCAACACCCGTGCGTTTTAT
TCTGTCTTTTTATTGCCGATCCCCTCAGAAGAACTCGTCAAGAAG
GCGATAGAAGGCGATGCGCTGCGAATCGGGAGCGGCGATACCGTA
AAGCACGAGGAAGCGGTCAGCCCATTCGCCGCCAAGCTCTTCAGC
AATATCACGGGTAGCCAACGCTATGTCCTGATAGCGGTCGGCCGC
TCATGTTCTCGTAGGAGTCGGCGTCCTCTTCGTGGTTAGGTCCAG
GTTGGCCTCTGATAGACCGCAGCTGAGGAGCGGCGTACAGAATGC
CTCTCATGTCCTCATAGCTGCCGCTGCCTTGTGGAGGCTTCTCGT
GCTTCAGTGTCTCGTATGTCTCTTGATTCCGGGTGCTCAGGCCGG
TGTACACGCCATCAGATTTCTCGTAGCTGGTGATGGCGGCCTTCC
GCACTTGGATCTTCAGCCGTCTGCAGTACAGGGTGATGACCAGAG
ACAGCAGCAGGACACCACATGTGCCAGCCAGAGGGGCCCAAATGT
AGATATCCAGGCCTCTGGTATGCACAGCTCCGCCTGCAGCAGGTC
TACAGGCTTCAGGTCTGAGAGACAGAGGCTGGCTGGCGATTGTAG
GAGCTGGTGTAGGTGGTCTAGGAGCGGGTGTTGTTGTAGGCTTGG
CGGGCAGAAACACGGGCACGAAGTGGCTGAAGTACATGATGCTAT
TGCTCAGGGCTCCGCTTCCTCCGCCGCCTGATTTGATTTCCAGCT
TGGTGCCTCCGCCAAATGTCCAAGGGCTCTCGTCGTACTGCTGGC
AGTAGTAGATGCCGAAGTCCTCGTACTGCAGGCTGCTGATTGTCA
GGGTGTAGTCGGTGCCAGAGCCGCTGCCAGAAAATCTGCTTGGCA
CGCCGCTTTCCAGTCTGTTGGCCCGGTAGATCAGTGTCTTAGGGG
CCTTGCCAGGCTTCTGCTGGAACCAGCTCAGGTAGCTGTTGATGT
CCTGGCTGGCTCTACAGGTGATGGTCACTCTATCGCCCACAGAGG
CAGACAGGCTGCTAGGGCTCTGTGTCATCTGGATATCAGAGCCAC
CACCGCCAGATCCACCGCCACCTGATCCTCCGCCTCCGCTAGAAA
CTGTCACTGTGGTGCCCTGGCCCCACACATCGAAGTACCAGTCGT
AGCCTCTTCTGGTGCAGAAGTACACGGCGGTATCCTCGGCTCTCA
GGCTGTTGATCTGCAGGTAGGCGGTGTTCTTGCTGTCGTCCAGGC
TGAAGGTGAATCTGCCCTTAAAGCTATCGGCGTAGGTTGGCTCGC
CGGTGTGGGTATTGATCCAGCCCATCCACTCAAGGCCAGGTGAGT
CCAGGAGATGTTTCAGCACTGTTGCCTTTAGTCTCGAGGCAACTT
AGACAACTGAGTATTGATCTGAGCACAGCAGGGTGTGAGCTGTTT
GAAGATACTGGGGTTGGGGGTGAAGAAACTGCAGAGGACTAACTG
GGCTGAGACCCAGTGGCAATGTTTTAGGGCCTAAGGAATGCCTCT
GAAAATCTAGATGGACAACTTTGACTTTGAGAAAAGAGAGGTGGA
AATGAGGAAAATGACTTTTCTTTATTAGATTTCGGTAGAAAGAAC
TTTCATCTTTCCCCTATTTTTGTTATTCGTTTTAAAACATCTATC
TGGAGGCAGGACAAGTATGGTCATTAAAAAGATGCAGGCAGAAGG
CATATATTGGCTCAGTCAAAGTGGGGAACTTTGGTGGCCAAACAT
ACATTGCTAAGGCTATTCCTATATCAGCTGGACACATATAAAATG
CTGCTAATGCTTCATTACAAACTTATATCCTTTAATTCCAGATGG
GGGCAAAGTATGTCCAGGGGTGAGGAACAATTGAAACATTTGGGC
TGGAGTAGATTTTGAAAGTCAGCTCTGTGTGTGTGTGTGTGTGTG
TGTGTGTGAGAGCGTGTGTTTCTTTTAACGTTTTCAGCCTACAGC
ATACAGGGTTCATGGTGGCAAGAAGATAACAAGATTTAAATTATG
GCCAGTGACTAGTGCTGCAAGAAGAACAACTACCTGCATTTAATG
GGAAAGCAAAATCTCAGGCTTTGAGGGAAGTTAACATAGGCTTGA
TTCTGGGTGGAAGCTGGGTGTGTAGTTATCTGGAGGCCAGGCTGG
AGCTCTCAGCTCACTATGGGTTCATCTTTATTGTCTCCTTTTTCC
AGGGGCCTGTCGGACCCAGTTCATGCCGTAGTTGGTGAAGGTGTA
GCCGCTGGCGGCACAGCTGATTCTGACAGATCCGCCAGGTTTCAC
AAGTCCGCCGCCAGACTGAACCAGCTGGATCTCAGAGATGCTACA
GGCCACTGTTCCCAGCAGCAGCAGAGACTGCAGCCACATCTGGTG
GCGAATTCGAAGCTTGAGCTCGAGATCTGAGTCCGGTAGCTGAAC
CGTCAGATCGCCGGCTAGCGGATCTGACGGTTCACTAAACCAGCT
CTGCTTATATAGACCTCCCACCGTACACGCCTACCGCCCATTTGC
GTCAATGGGGCGGAGTTGTTACGACATTTTGGAAAGTCCCGTTGA
TTTTGGTGCCAAAACAAACTCCCATTGACGTCAATGGGGTGGAGA
CTTGGAAATCCCCGTGAGTCAAACCGCTATCCACGCCCATTGATG
TACTGCCAAAACCGCATCACCATGGTAATAGCGATGACTAATACG
TAGATGTACTGCCAAGTAGGAAAGTCCCATAAGGTCATGTACTGG
GCATAATGCCAGGCGGGCCATTTACCGTCATTGACGTCAATAGGG
GGCGTACTTGGCATATGATACACTTGATGTACTGCCAAGTGGGCA
GTTTACCGTAAATACTCCACCCATTGACGTCAATGGAAAGTCCCT
ATTGGCGTTACTATGGGAACATACGTCATTATTGACGTCAATGGG
CGGGGGTCGTTGGGCGGTCAGCCAGGCGGGCCATTTACCGTAAGT
TATGTAACGCGGAACTCCATATATGGGCTATGAACTAATGACCCC
GTAATTGATTACTATTAGCCCGGGGGATCCAGACATGATAAGATA
CATTGATGAGTTTGGACAAACCACAACTAGAATGCAGTGAAAAAA
ATGCTTTATTTGTGAAATTTGTGATGCTATTGCTTTATTTGTAAC
CATTATAAGCTGCAATAAACAAGTTAACAACAACAATTGCATTCA
TTTTATGTTTCAGGTTCAGGGGGAGGTGTGGGAGGTTTTTTAAAG
CAAGTAAAACCTCTACAAATGTGGTATGGCTGATTATGATCCGGC
TGCCTCGCGCGTTTCGGTGATGACGGTGAAAACCTCTGACACATG
CAGCTCCCGGAGACGGTCACAGCTTGTCTGTAAGCGGATGCCGGG
AGCAGACAAGCCCGTCAGGGCGCGTCAGCGGGTGTTGGCGGGTGT
CGGGGCGCAGCCATGAGGTCGATCGACTCTAGAGGATCGATCCCC
GCCCCGGACGAACTAAACCTGACTACGACATCTCTGCCCCTTCTT
CGCGGGGCAGTGCATGTAATCCCTTCAGTTGGTTGGTACAACTTG
CCAACTGGGCCCTGTTCCACATGTGACACGGGGGGGGACCAAACA
CAAAGGGGTTCTCTGACTGTAGTTGACATCCTTATAAATGGATGT
GCACATTTGCCAACACTGAGTGGCTTTCATCCTGGAGCAGACTTT
GCAGTCTGTGGACTGCAACACAACATTGCCTTTATGTGTAACTCT
TGGCTGAAGCTCTTACACCAATGCTGGGGGACATGTACCTCCCAG
GGGCCCAGGAAGACTACGGGAGGCTACACCAACGTCAATCAGAGG
GGCCTGTGTAGCTACCGATAAGCGGACCCTCAAGAGGGCATTAGC
AATAGTGTTTATAAGGCCCCCTTGTTAACCCTAAACGGGTAGCAT
ATGCTTCCCGGGTAGTAGTATATACTATCCAGACTAACCCTAATT
CAATAGCATATGTTACCCAACGGGAAGCATATGCTATCGAATTAG
GGTTAGTAAAAGGGTCCTAAGGAACAGCGATATCTCCCACCCCAT
GAGCTGTCACGGTTTTATTTACATGGGGTCAGGATTCCACGAGGG
TAGTGAACCATTTTAGTCACAAGGGCAGTGGCTGAAGATCAAGGA
GCGGGCAGTGAACTCTCCTGAATCTTCGCCTGCTTCTTCATTCTC
CTTCGTTTAGCTAATAGAATAACTGCTGAGTTGTGAACAGTAAGG
TGTATGTGAGGTGCTCGAAAACAAGGTTTCAGGTGACGCCCCCAG
AATAAAATTTGGACGGGGGGTTCAGTGGTGGCATTGTGCTATGAC
ACCAATATAACCCTCACAAACCCCTTGGGCAATAAATACTAGTGT
AGGAATGAAACATTCTGAATATCTTTAACAATAGAAATCCATGGG
GTGGGGACAAGCCGTAAAGACTGGATGTCCATCTCACACGAATTT
ATGGCTATGGGCAACACATAATCCTAGTGCAATATGATACTGGGG
TTATTAAGATGTGTCCCAGGCAGGGACCAAGACAGGTGAACCATG
TTGTTACACTCTATTTGTAACAAGGGGAAAGAGAGTGGACGCCGA
CAGCAGCGGACTCCACTGGTTGTCTCTAACACCCCCGAAAATTAA
ACGGGGCTCCACGCCAATGGGGCCCATAAACAAAGACAAGTGGCC
ACTCTTTTTTTTGAAATTGTGGAGTGGGGGCACGCGTCAGCCCCC
ACACGCCGCCCTGCGGTTTTGGACTGTAAAATAAGGGTGTAATAA
CTTGGCTGATTGTAACCCCGCTAACCACTGCGGTCAAACCACTTG
CCCACAAAACCACTAATGGCACCCCGGGGAATACCTGCATAAGTA
GGTGGGCGGGCCAAGATAGGGGCGCGATTGCTGCGATCTGGAGGA
CAAATTACACACACTTGCGCCTGAGCGCCAAGCACAGGGTTGTTG
GTCCTCATATTCACGAGGTCGCTGAGAGCACGGTGGGCTAATGTT
GCCATGGGTAGCATATACTACCCAAATATCTGGATAGCATATGCT
ATCCTAATCTATATCTGGGTAGCATAGGCTATCCTAATCTATATC
TGGGTAGCATATGCTATCCTAATCTATATCTGGGTAGTATATGCT
ATCCTAATTTATATCTGGGTAGCATAGGCTATCCTAATCTATATC
TGGGTAGCATATGCTATCCTAATCTATATCTGGGTAGTATATGCT
ATCCTAATCTGTATCCGGGTAGCATATGCTATCCTAATAGAGATT
AGGGTAGTATATGCTATCCTAATTTATATCTGGGTAGCATATACT
ACCCAAATATCTGGATAGCATATGCTATCCTAATCTATATCTGGG
TAGCATATGCTATCCTAATCTATATCTGGGTAGCATAGGCTATCC
TAATCTATATCTGGGTAGCATATGCTATCCTAATCTATATCTGGG
TAGTATATGCTATCCTAATTTATATCTGGGTAGCATAGGCTATCC
TAATCTATATCTGGGTAGCATATGCTATCCTAATCTATATCTGGG
TAGTATATGCTATCCTAATCTGTATCCGGGTAGCATATGCTATCC
TCATGCATATACAGTCAGCATATGATACCCAGTAGTAGAGTGGGA
GTGCTATCCTTTGCATATGCCGCCACCTCCCAAGGGGGCGTGAAT
TTTCGCTGCTTGTCCTTTTCCTGCATGCTGGTTGCTCCCATTCTT
AGGTGAATTTAAGGAGGCCAGGCTAAAGCCGTCGCATGTCTGATT
GCTCACCAGGTAAATGTCGCTAATGTTTTCCAACGCGAGAAGGTG
TTGAGCGCGGAGCTGAGTGACGTGACAACATGGGTATGCCCAATT
GCCCCATGTTGGGAGGACGAAAATGGTGACAAGACAGATGGCCAG
AAATACACCAACAGCACGCATGATGTCTACTGGGGATTTATTCTT
TAGTGCGGGGGAATACACGGCTTTTAATACGATTGAGGGCGTCTC
CTAACAAGTTACATCACTCCTGCCCTTCCTCACCCTCATCTCCAT
CACCTCCTTCATCTCCGTCATCTCCGTCATCACCCTCCGCGGCAG
CCCCTTCCACCATAGGTGGAAACCAGGGAGGCAAATCTACTCCAT
CGTCAAAGCTGCACACAGTCACCCTGATATTGCAGGTAGGAGCGG
GCTTTGTCATAACAAGGTCCTTAATCGCATCCTTCAAAACCTCAG
CAAATATATGAGTTTGTAAAAAGACCATGAAATAACAGACAATGG
ACTCCCTTAGCGGGCCAGGTTGTGGGCCGGGTCCAGGGGCCATTC
CAAAGGGGAGACGACTCAATGGTGTAAGACGACATTGTGGAATAG
CAAGGGCAGTTCCTCGCCTTAGGTTGTAAAGGGAGGTCTTACTAC
CTCCATATACGAACACACCGGCGACCCAAGTTCCTTCGTCGGTAG
TCCTTTCTACGTGACTCCTAGCCAGGAGAGCTCTTAAACCTTCTG
CAATGTTCTCAAATTTCGGGTTGGAACCTCCTTGACCACGATGCT
TTCCAAACCACCCTCCTTTTTTGCGCCTGCCTCCATCACCCTGAC
CCCGGGGTCCAGTGCTTGGGCCTTCTCCTGGGTCATCTGCGGGGC
CCTGCTCTATCGCTCCCGGGGGCACGTCAGGCTCACCATCTGGGC
CACCTTCTTGGTGGTATTCAAAATAATCGGCTTCCCCTACAGGGT
GGAAAAATGGCCTTCTACCTGGAGGGGGCCTGCGCGGTGGAGACC
CGGATGATGATGACTGACTACTGGGACTCCTGGGCCTCTTTTCTC
CACGTCCACGACCTCTCCCCCTGGCTCTTTCACGACTTCCCCCCC
TGGCTCTTTCACGTCCTCTACCCCGGCGGCCTCCACTACCTCCTC
GACCCCGGCCTCCACTACCTCCTCGACCCCGGCCTCCACTGCCTC
CTCGACCCCGGCCTCCACCTCCTGCTCCTGCCCCTCCTGCTCCTG
CCCCTCCTCCTGCTCCTGCCCCTCCTGCCCCTCCTGCTCCTGCCC
CTCCTGCCCCTCCTGCTCCTGCCCCTCCTGCCCCTCCTGCTCCTG
CCCCTCCTGCCCCTCCTCCTGCTCCTGCCCCTCCTGCCCCTCCTC
CTGCTCCTGCCCCTCCTGCCCCTCCTGCTCCTGCCCCTCCTGCCC
CTCCTGCTCCTGCCCCTCCTGCCCCTCCTGCTCCTGCCCCTCCTG
CTCCTGCCCCTCCTGCTCCTGCCCCTCCTGCTCCTGCCCCTCCTG
CCCCTCCTGCCCCTCCTCCTGCTCCTGCCCCTCCTGCTCCTGCCC
CTCCTGCCCCTCCTGCCCCTCCTGCTCCTGCCCCTCCTCCTGCTC
CTGCCCCTCCTGCCCCTCCTGCCCCTCCTCCTGCTCCTGCCCCTC
CTGCCCCTCCTCCTGCTCCTGCCCCTCCTCCTGCTCCTGCCCCTC
CTGCCCCTCCTGCCCCTCCTCCTGCTCCTGCCCCTCCTGCCCCTC
CTCCTGCTCCTGCCCCTCCTCCTGCTCCTGCCCCTCCTGCCCCTC
CTGCCCCTCCTCCTGCTCCTGCCCCTCCTCCTGCTCCTGCCCCTC
CTGCCCCTCCTGCCCCTCCTGCCCCTCCTCCTGCTCCTGCCCCTC
CTCCTGCTCCTGCCCCTCCTGCTCCTGCCCCTCCCGCTCCTGCTC
CTGCTCCTGTTCCACCGTGGGTCCCTTTGCAGCCAATGCAACTTG
GACGTTTTTGGGGTCTCCGGACACCATCTCTATGTCTTGGCCCTG
ATCCTGAGCCGCCCGGGGCTCCTGGTCTTCCGCCTCCTCGTCCTC
GTCCTCTTCCCCGTCCTCGTCCATGGTTATCACCCCCTCTTCTTT
GAGGTCCACTGCCGCCGGAGCCTTCTGGTCCAGATGTGTCTCCCT
TCTCTCCTAGGCCATTTCCAGGTCCTGTACCTGGCCCCTCGTCAG
ACATGATTCACACTAAAAGAGATCAATAGACATCTTTATTAGACG
ACGCTCAGTGAATACAGGGAGTGCAGACTCCTGCCCCCTCCAACA
GCCCCCCCACCCTCATCCCCTTCATGGTCGCTGTCAGACAGATCC
AGGTCTGAAAATTCCCCATCCTCCGAACCATCCTCGTCCTCATCA
CCAATTACTCGCAGCCCGGAAAACTCCCGCTGAACATCCTCAAGA
TTTGCGTCCTGAGCCTCAAGCCAGGCCTCAAATTCCTCGTCCCCC
TTTTTGCTGGACGGTAGGGATGGGGATTCTCGGGACCCCTCCTCT
TCCTCTTCAAGGTCACCAGACAGAGATGCTACTGGGGCAACGGAA
GAAAAGCTGGGTGCGGCCTGTGAGGATCAGCTTATCGATGATAAG
CTGTCAAACATGAGAATTCTTGAAGACGAAAGGGCCTCGTGATAC
GCCTATTTTTATAGGTTAATGTCATGATAATAATGGTTTCTTAGA
CGTCAGGTGGCACTTTTCGGGGAAATGTGCGCGGAACCCCTATTT
GTTTATTTTTCTAAATACATTCAAATATGTATCCGCTCATGAGAC
AATAACCCTGATAAATGCTTCAATAATATTGAAAAAGGAAGAGTA
TGAGTATTCAACATTTCCGTGTCGCCCTTATTCCCTTTTTTGCGG
CATTTTGCCTTCCTGTTTTTGCTCACCCAGAAACGCTGGTGAAAG
TAAAAGATGCTGAAGATCAGTTGGGTGCACGAGTGGGTTACATCG
AACTGGATCTCAACAGCGGTAAGATCCTTGAGAGTTTTCGCCCCG
AAGAACGTTTTCCAATGATGAGCACTTTTAAAGTTCTGCTATGTG
GCGCGGTATTATCCCGTGTTGACGCCGGGCAAGAGCAACTCGGTC
GCCGCATACACTATTCTCAGAATGACTTGGTTGAGTACTCACCAG
TCACAGAAAAGCATCTTACGGATGGCATGACAGTAAGAGAATTAT
GCAGTGCTGCCATAACCATGAGTGATAACACTGCGGCCAACTTAC
TTCTGACAACGATCGGAGGACCGAAGGAGCTAACCGCTTTTTTGC
ACAACATGGGGGATCATGTAACTCGCCTTGATCGTTGGGAACCGG
AGCTGAATGAAGCCATACCAAACGACGAGCGTGACACCACGATGC
CTGCAGCAATGGCAACAACGTTGCGCAAACTATTAACTGGCGAAC
TACTTACTCTAGCTTCCCGGCAACAATTAATAGACTGGATGGAGG
CGGATAAAGTTGCAGGACCACTTCTGCGCTCGGCCCTTCCGGCTG
GCTGGTTTATTGCTGATAAATCTGGAGCCGGTGAGCGTGGGTCTC
GCGGTATCATTGCAGCACTGGGGCCAGATGGTAAGCCCTCCCGTA
TCGTAGTTATCTACACGACGGGGAGTCAGGCAACTATGGATGAAC
GAAATAGACAGATCGCTGAGATAGGTGCCTCACTGATTAAGCATT
GGTAACTGTCAGACCAAGTTTACTCATATATACTTTAGATTGATT
TAAAACTTCATTTTTAATTTAAAAGGATCTAGGTGAAGATCCTTT
TTGATAATCTCATGACCAAAATCCCTTAACGTGAGTTTTCGTTCC
ACTGAGCGTCAGACCCCGTAGAAAAGATCAAAGGATCTTCTTGAG
ATCCTTTTTTTCTGCGCGTAATCTGCTGCTTGCAAACAAAAAAAC
CACCGCTACCAGCGGTGGTTTGTTTGCCGGATCAAGAGCTACCAA
CTCTTTTTCCGAAGGTAACTGGCTTCAGCAGAGCGCAGATACCAA
ATACTGTCCTTCTAGTGTAGCCGTAGTTAGGCCACCACTTCAAGA
ACTCTGTAGCACCGCCTACATACCTCGCTCTGCTAATCCTGTTAC
CAGTGGCTGCTGCCAGTGGCGATAAGTCGTGTCTTACCGGGTTGG
ACTCAAGACGATAGTTACCGGATAAGGCGCAGCGGTCGGGCTGAA
CGGGGGGTTCGTGCACACAGCCCAGCTTGGAGCGAACGACCTACA
CCGAACTGAGATACCTACAGCGTGAGCTATGAGAAAGCGCCACGC
TTCCCGAAGGGAGAAAGGCGGACAGGTATCCGGTAAGCGGCAGGG
TCGGAACAGGAGAGCGCACGAGGGAGCTTCCAGGGGGAAACGCCT
GGTATCTTTATAGTCCTGTCGGGTTTCGCCACCTCTGACTTGAGC
GTCGATTTTTGTGATGCTCGTCAGGGGGGCGGAGCCTATGGAAAA
ACGCCAGCAACGCGGCCTTTTTACGGTTCCTGGCCTTTTGCTGGC
CTTTTGCTCACATGTTCTTTCCTGCGTTATCCCCTGATTCTGTGG
ATAACCGTATTACCGCCTTTGAGTGAGCTGATACCGCTCGCCGCA
GCCGAACGACCGAGCGCAGCGAGTCAGTGAGCGAGGAAGCGGAAG
AGCGCCTGATGCGGTATTTTCTCCTTACGCATCTGTGCGGTATTT
CACACCGCATATGGTGCACTCTCAGTACAATCTGCTCTGATGCCG
CATAGTTAAGCCAGCTGTGGAATGTGTGTCAGTTAGGGTGTGGAA
AGTCCCCAGGCTCCCCAGCAGGCAGAAGTATGCAAAGCATGCATC
TCAATTAGTCAGCAACCAGGTGTGGAAAGTCCCCAGGCTCCCCAG
CAGGCAGAAGTATGCAAAGCATGCATCTCAATTAGTCAGCAACCA
TAGTCCCGCCCCTAACTCCGCCCATCCCGCCCCTAACTCCGCCCA
GTTCCGCCCATTCTCCGCCCCATGGCTGACTAATTTTTTTTATTT
ATGCAGAGGCCGAGGCCGCCTCGGCCTCTGAGCTATTCCAGAAGT
AGTGAGGAGGCTTTTTTGGAGGCCTAGGCTTTTGCAAAAAGCTTG
CATGCCTGCAGGTCGGCCGCCACGACCGGTGCCGCCACCATCCCC
TGACCCACGCCCCTGACCCCTCACAAGGAGACGACCTTCCATGAC
CGAGTACAAGCCCACGGTGCGCCTCGCCACCCGCGACGACGTCCC
CCGGGCCGTACGCACCCTCGCCGCCGCGTTCGCCGACTACCCCGC
CACGCGCCACACCGTCGACCCGGACCGCCACATCGAGCGGGTCAC
CGAGCTGCAAGAACTCTTCCTCACGCGCGTCGGGCTCGACATCGG
CAAGGTGTGGGTCGCGGACGACGGCGCCGCGGTGGCGGTCTGGAC
CACGCCGGAGAGCGTCGAAGCGGGGGCGGTGTTCGCCGAGATCGG
CCCGCGCATGGCCGAGTTGAGCGGTTCCCGGCTGGCCGCGCAGCA
ACAGATGGAAGGCCTCCTGGCGCCGCACCGGCCCAAGGAGCCCGC
GTGGTTCCTGGCCACCGTCGGCGTCTCGCCCGACCACCAGGGCAA
GGGTCTGGGCAGCGCCGTCGTGCTCCCCGGAGTGGAGGCGGCCGA
GCGCGCCGGGGTGCCCGCCTTCCTGGAGACCTCCGCGCCCCGCAA
CCTCCCCTTCTACGAGCGGCTCGGCTTCACCGTCACCGCCGACGT
CGAGGTGCCCGAAGGACCGCGCACCTGGTGCATGACCCGCAAGCC
CGGTGCCTGACGCCCGCCCCACGACCCGCAGCGCCCGACCGAAAG
GAGCGCACGACCCCATGGCTCCGACCGAAGCCGACCCGGGCGGCC
CCGCCGACCCCGCACCCGCCCCCGAGGCCCACCGACTCTAGAGGA
TCATAATCAGCCATACCACATTTGTAGAGGTTTTACTTGCTTTAA
AAAACCTCCCACACCTCCCCCTGAACCTGAAACATAAAATGAATG
CAATTGTTGTTGTTAACTTGTTTATTGCAGCTTATAATGGTTACA
AATAAAGCAATAGCATCACAAATTTCACAAATAAAGCATTTTTTT
CACTGCATTCTAGTTGTGGTTTGTCCAAACTCATCAATGTATCTT
ATCATGTCTGGATCACTCGCCGATAGTGGAAACCGACGCCCCAGC
ACTCGTCCGAGGGCAAAGGAATAGGGGAGATGGGGGAGGCTAACT
GAAACACGGAAGGAGACAATACCGGAAGGAACCCGCGCTATGACG
GCAATAAAAAGACAGAATAAAACGCACGGGTGTTGGGTCGTTTGT
TCATAAACGCGGGGTTCGGTCCCAGGGCTGGCACTCTGTCGATAC
CCCACCGAGACCCCATTGGGGCCAATACGCCCGCGTTTCTTCCTT
TTCCCCACCCCACCCCCCAAGTTCGGGTGAAGGCCCAGGGCTCGC
AGCCAACGTCGGGGCGGCAGGCCCTGCCATAGCCACTGGCCCCGT
GGGTTAGGGACGGGGTCCCCCATGGGGAATGGTTTATGGTTCGTG
GGGGTTATTATTTTGGGCGTTGCGTGGGGTCTGGTCCACGACTGG
ACTGAGCAGACAGACCCATGGTTTTTGGATGGCCTGGGCATGGAC
CGCATGTACTGGCGCGACACGAACACCGGGCGTCTGTGGCTGCCA
AACACCCCCGACCCCCAAAAACCACCGCGCGGATTTCTGGCGTGC
CAAGCTAGTCGACCAATTCTCATGTTTGACAGCTTATCATCGCAG
ATCCGGGCAACGTTGTTGCATTGCTGCAGGCGCAGAACTGGTAGG
TATGGAAGATCTCTAGAAGCTGGGTACCAGCTGCTAGCAAGCTTG
CTAGCGGCCGGCTCGAGTTTACTCCCTATCAGTGATAGAGAACGT
ATGTCGAGTTTACTCCCTATCAGTGATAGAGAACGATGTCGAGTT
TACTCCCTATCAGTGATAGAGAACGTATGTCGAGTTTACTCCCTA
TCAGTGATAGAGAACGTATGTCGAGTTTACTCCCTATCAGTGATA
GAGAACGTATGTCGAGTTTATCCCTATCAGTGATAGAGAACGTAT
GTCGAGTTTACTCCCTATCAGTGATAGAGAACGTATGTCGAGGTA
GGCGTGTACGGTGGGAGGCCTATATAAGCAGAGCTCGTTTAG
(SEQ ID NO: 39)
LINE-1 plasmid-CD5_FCR-PI3K_T2A-GFP intron
(SEQ ID NO: 40)
CGGCCGCGGGGGGAGGAGCCAAGATGGCCGAATAGGAACAGCTCC
GGTCTACAGCTCCCAGCGTGAGCGACGCAGAAGACGGTGATTTCT
GCATTTCCATCTGAGGTACCGGGTTCATCTCACTAGGGAGTGCCA
GACAGTGGGCGCAGGCCAGTGTGTGTGCGCACCGTGCGCGAGCCG
AAGCAGGGCGAGGCATTGCCTCACCTGGGAAGCGCAAGGGGTCAG
GGAGTTCCCTTTCCGAGTCAAAGAAAGGGGTGACGGACGCACCTG
GAAAATCGGGTCACTCCCACCCGAATATTGCGCTTTTCAGACCGG
CTTAAGAAACGGCGCACCACGAGACTATATCCCACACCTGGCTCG
GAGGGTCCTACGCCCACGGAATCTCGCTGATTGCTAGCACAGCAG
TCTGAGATCAAACTGCAAGGCGGCAACGAGGCTGGGGGAGGGGCG
CCCGCCATTGCCCAGGCTTGCTTAGGTAAACAAAGCAGCAGGGAA
GCTCGAACTGGGTGGAGCCCACCACAGCTCAAGGAGGCCTGCCTG
CCTCTGTAGGCTCCACCTCTGGGGGCAGGGCACAGACAAACAAAA
AGACAGCAGTAACCTCTGCAGACTTAAGTGTCCCTGTCTGACAGC
TTTGAAGAGAGCAGTGGTTCTCCCAGCACGCAGCTGGAGATCTGA
GAACGGGCAGACTGCCTCCTCAAGTGGGTCCCTGACCCCTGACCC
CCGAGCAGCCTAACTGGGAGGCACCCCCCAGCAGGGGCACACTGA
CACCTCACACGGCAGGGTATTCCAACAGACCTGCAGCTGAGGGTC
CTGTCTGTTAGAAGGAAAACTAACAACCAGAAAGGACATCTACAC
CGAAAACCCATCTGTACATCACCATCATCAAAGACCAAAAGTAGA
TAAAACCACAAAGATGGGGAAAAAACAGAACAGAAAAACTGGAAA
CTCTAAAACGCAGAGCGCCTCTCCTCCTCCAAAGGAACGCAGTTC
CTCACCAGCAACAGAACAAAGCTGGATGGAGAATGATTTTGATGA
GCTGAGAGAAGAAGGCTTCAGACGATCAAATTACTCTGAGCTACG
GGAGGACATTCAAACCAAAGGCAAAGAAGTTGAAAACTTTGAAAA
AAATTTAGAAGAATGTATAACTAGAATAACCAATACAGAGAAGTG
CTTAAAGGAGCTGATGGAGCTGAAAACCAAGGCTCGAGAACTACG
TGAAGAATGCAGAAGCCTCAGGAGCCGATGCGATCAACTGGAAGA
AAGGGTATCAGCAATGGAAGATGAAATGAATGAAATGAAGCGAGA
AGGGAAGTTTAGAGAAAAAAGAATAAAAAGAAATGAGCAAAGCCT
CCAAGAAATATGGGACTATGTGAAAAGACCAAATCTACGTCTGAT
TGGTGTACCTGAAAGTGATGTGGAGAATGGAACCAAGTTGGAAAA
CACTCTGCAGGATATTATCCAGGAGAACTTCCCCAATCTAGCAAG
GCAGGCCAACGTTCAGATTCAGGAAATACAGAGAACGCCACAAAG
ATACTCCTCGAGAAGAGCAACTCCAAGACACATAATTGTCAGATT
CACCAAAGTTGAAATGAAGGAAAAAATGTTAAGGGCAGCCAGAGA
GAAAGGTCGGGTTACCCTCAAAGGAAAGCCCATCAGACTAACAGC
GGATCTCTCGGCAGAAACCCTACAAGCCAGAAGAGAGTGGGGGCC
AATATTCAACATTCTTAAAGAAAAGAATTTTCAACCCAGAATTTC
ATATCCAGCCAAACTAAGCTTCATAAGTGAAGGAGAAATAAAATA
CTTTATAGACAAGCAAATGTTGAGAGATTTTGTCACCACCAGGCC
TGCCCTAAAAGAGCTCCTGAAGGAAGCGCTAAACATGGAAAGGAA
CAACCGGTACCAGCCGCTGCAAAATCATGCCAAAATGTAAAGACC
ATCAAGACTAGGAAGAAACTGCATCAACTAATGAGCAAAATCACC
AGCTAACATCATAATGACAGGATCAACTTCACACATAACAATATT
AACTTTAAATATAAATGGACTAAATTCTGCAATTAAAAGACACAG
ACTGGCAAGTTGGATAAAGAGTCAAGACCCATCAGTGTGCTGTAT
TCAGGAAACCCATCTCACGTGCAGAGACACACATAGGCTCAAAAT
AAAAGGATGGAGGAAGATCTACCAAGCCAATGGAAAACAAAAAAA
GGCAGGGGTTGCAATCCTAGTCTCTGATAAAACAGACTTTAAACC
AACAAAGATCAAAAGAGACAAAGAAGGCCATTACATAATGGTAAA
GGGATCAATTCAACAAGAGGAGCTAACTATCCTAAATATTTATGC
ACCCAATACAGGAGCACCCAGATTCATAAAGCAAGTCCTCAGTGA
CCTACAAAGAGACTTAGACTCCCACACATTAATAATGGGAGACTT
TAACACCCCACTGTCAACATTAGACAGATCAACGAGACAGAAAGT
CAACAAGGATACCCAGGAATTGAACTCAGCTCTGCACCAAGCAGA
CCTAATAGACATCTACAGAACTCTCCACCCCAAATCAACAGAATA
TACATTTTTTTCAGCACCACACCACACCTATTCCAAAATTGACCA
CATAGTTGGAAGTAAAGCTCTCCTCAGCAAATGTAAAAGAACAGA
AATTATAACAAACTATCTCTCAGACCACAGTGCAATCAAACTAGA
ACTCAGGATTAAGAATCTCACTCAAAGCCGCTCAACTACATGGAA
ACTGAACAACCTGCTCCTGAATGACTACTGGGTACATAACGAAAT
GAAGGCAGAAATAAAGATGTTCTTTGAAACCAACGAGAACAAAGA
CACCACATACCAGAATCTCTGGGACGCATTCAAAGCAGTGTGTAG
AGGGAAATTTATAGCACTAAATGCCTACAAGAGAAAGCAGGAAAG
ATCCAAAATTGACACCCTAACATCACAATTAAAAGAACTAGAAAA
GCAAGAGCAAACACATTCAAAAGCTAGCAGAAGGCAAGAAATAAC
TAAAATCAGAGCAGAACTGAAGGAAATAGAGACACAAAAAACCCT
TCAAAAAATCAATGAATCCAGGAGCTGGTTTTTTGAAAGGATCAA
CAAAATTGATAGACCGCTAGCAAGACTAATAAAGAAAAAAAGAGA
GAAGAATCAAATAGACACAATAAAAAATGATAAAGGGGATATCAC
CACCGATCCCACAGAAATACAAACTACCATCAGAGAATACTACAA
ACACCTCTACGCAAATAAACTAGAAAATCTAGAAGAAATGGATAC
ATTCCTCGACACATACACTCTCCCAAGACTAAACCAGGAAGAAGT
TGAATCTCTGAATCGACCAATAACAGGCTCTGAAATTGTGGCAAT
AATCAATAGTTTACCAACCAAAAAGAGTCCAGGACCAGATGGATT
CACAGCCGAATTCTACCAGAGGTACAAGGAGGAACTGGTACCATT
CCTTCTGAAACTATTCCAATCAATAGAAAAAGAGGGAATCCTCCC
TAACTCATTTTATGAGGCCAGCATCATTCTGATACCAAAGCCGGG
CAGAGACACAACCAAAAAAGAGAATTTTAGACCAATATCCTTGAT
GAACATTGATGCAAAAATCCTCAATAAAATACTGGCAAACCGAAT
CCAGCAGCACATCAAAAAGCTTATCCACCATGATCAAGTGGGCTT
CATCCCTGGGATGCAAGGCTGGTTCAATATACGCAAATCAATAAA
TGTAATCCAGCATATAAACAGAGCCAAAGACAAAAACCACATGAT
TATCTCAATAGATGCAGAAAAAGCCTTTGACAAAATTCAACAACC
CTTCATGCTAAAAACTCTCAATAAATTAGGTATTGATGGGACGTA
TTTCAAAATAATAAGAGCTATCTATGACAAACCCACAGCCAATAT
CATACTGAATGGGCAAAAACTGGAAGCATTCCCTTTGAAAACCGG
CACAAGACAGGGATGCCCTCTCTCACCGCTCCTATTCAACATAGT
GTTGGAAGTTCTGGCCAGGGCAATCAGGCAGGAGAAGGAAATAAA
GGGTATTCAATTAGGAAAAGAGGAAGTCAAATTGTCCCTGTTTGC
AGACGACATGATTGTTTATCTAGAAAACCCCATCGTCTCAGCCCA
AAATCTCCTTAAGCTGATAAGCAACTTCAGCAAAGTCTCAGGATA
CAAAATCAATGTACAAAAATCACAAGCATTCTTATACACCAACAA
CAGACAAACAGAGAGCCAAATCATGGGTGAACTCCCATTCACAAT
TGCTTCAAAGAGAATAAAATACCTAGGAATCCAACTTACAAGGGA
TGTGAAGGACCTCTTCAAGGAGAACTACAAACCACTGCTCAAGGA
AATAAAAGAGGAGACAAACAAATGGAAGAACATTCCATGCTCATG
GGTAGGAAGAATCAATATCGTGAAAATGGCCATACTGCCCAAGGT
AATTTACAGATTCAATGCCATCCCCATCAAGCTACCAATGACTTT
CTTCACAGAATTGGAAAAAACTACTTTAAAGTTCATATGGAACCA
AAAAAGAGCCCGCATTGCCAAGTCAATCCTAAGCCAAAAGAACAA
AGCTGGAGGCATCACACTACCTGACTTCAAACTATACTACAAGGC
TACAGTAACCAAAACAGCATGGTACTGGTACCAAAACAGAGATAT
AGATCAATGGAACAGAACAGAGCCCTCAGAAATAATGCCGCATAT
CTACAACTATCTGATCTTTGACAAACCTGAGAAAAACAAGCAATG
GGGAAAGGATTCCCTATTTAATAAATGGTGCTGGGAAAACTGGCT
AGCCATATGTAGAAAGCTGAAACTGGATCCCTTCCTTACACCTTA
TACAAAAATCAATTCAAGATGGATTAAAGATTTAAACGTTAAACC
TAAAACCATAAAAACCCTAGAAGAAAACCTAGGCATTACCATTCA
GGACATAGGCGTGGGCAAGGACTTCATGTCCAAAACACCAAAAGC
AATGGCAACAAAAGACAAAATTGACAAATGGGATCTAATTAAACT
AAAGAGCTTCTGCACAGCAAAAGAAACTACCATCAGAGTGAACAG
GCAACCTACAACATGGGAGAAAATTTTTGCAACCTACTCATCTGA
CAAAGGGCTAATATCCAGAATCTACAATGAACTCAAACAAATTTA
CAAGAAAAAAACAAACAACCCCATCAAAAAGTGGGCGAAGGACAT
GAACAGACACTTCTCAAAAGAAGACATTTATGCAGCCAAAAAACA
CATGAAGAAATGCTCATCATCACTGGCCATCAGAGAAATGCAAAT
CAAAACCACTATGAGATATCATCTCACACCAGTTAGAATGGCAAT
CATTAAAAAGTCAGGAAACAACAGGTGCTGGAGAGGATGCGGAGA
AATAGGAACACTTTTACACTGTTGGTGGGACTGTAAACTAGTTCA
ACCATTGTGGAAGTCAGTGTGGCGATTCCTCAGGGATCTAGAACT
AGAAATACCATTTGACCCAGCCATCCCATTACTGGGTATATACCC
AAATGAGTATAAATCATGCTGCTATAAAGACACATGCACACGTAT
GTTTATTGCGGCACTATTCACAATAGCAAAGACTTGGAACCAACC
CAAATGTCCAACAATGATAGACTGGATTAAGAAAATGTGGCACAT
ATACACCATGGAATACTATGCAGCCATAAAAAATGATGAGTTCAT
ATCCTTTGTAGGGACATGGATGAAATTGGAAACCATCATTCTCAG
TAAACTATCGCAAGAACAAAAAACCAAACACCGCATATTCTCACT
CATAGGTGGGAATTGAACAATGAGATCACATGGACACAGGAAGGG
GAATATCACACTCTGGGGACTGTGGTGGGGTCGGGGGAGGGGGGA
GGGATAGCATTGGGAGATATACCTAATGCTAGATGACACATTAGT
GGGTGCAGCGCACCAGCATGGCACATGTATACGGATCCGAATTCT
CGACGGATCGATCCGAACAAACGACCCAACACCCGTGCGTTTTAT
TCTGTCTTTTTATTGCCGATCCCCTCAGAAGAACTCGTCAAGAAG
GCGATAGAAGGCGATGCGCTGCGAATCGGGAGCGGCGATACCGTA
AAGCACGAGGAAGCGGTCAGCCCATTCGCCGCCAAGCTCTTCAGC
AATATCACGGGTAGCCAACGCTATGTCCTGATAGCGGTCGGCCGC
TTTACTTGTACAGCTCGTCCATGCCGAGAGTGATCCCGGCGGCGG
TCACGAACTCCAGCAGGACCATGTGATCGCGCTTCTCGTTGGGGT
CTTTGCTCAGGGCGGACTGGGTGCTCAGGTAGTGGTTGTCGGGCA
GCAGCACGGGGCCGTCGCCGATGGGGGTGTTCTGCTGGTAGTGGT
CGGCCAGGTGAGTCCAGGAGATGTTTCAGCACTGTTGCCTTTAGT
CTCGAGGCAACTTAGACAACTGAGTATTGATCTGAGCACAGCAGG
GTGTGAGCTGTTTGAAGATACTGGGGTTGGGGGTGAAGAAACTGC
AGAGGACTAACTGGGCTGAGACCCAGTGGCAATGTTTTAGGGCCT
AAGGAATGCCTCTGAAAATCTAGATGGACAACTTTGACTTTGAGA
AAAGAGAGGTGGAAATGAGGAAAATGACTTTTCTTTATTAGATTT
CGGTAGAAAGAACTTTCATCTTTCCCCTATTTTTGTTATTCGTTT
TAAAACATCTATCTGGAGGCAGGACAAGTATGGTCATTAAAAAGA
TGCAGGCAGAAGGCATATATTGGCTCAGTCAAAGTGGGGAACTTT
GGTGGCCAAACATACATTGCTAAGGCTATTCCTATATCAGCTGGA
CACATATAAAATGCTGCTAATGCTTCATTACAAACTTATATCCTT
TAATTCCAGATGGGGGCAAAGTATGTCCAGGGGTGAGGAACAATT
GAAACATTTGGGCTGGAGTAGATTTTGAAAGTCAGCTCTGTGTGT
GTGTGTGTGTGTGTGTGTGTGAGAGCGTGTGTTTCTTTTAACGTT
TTCAGCCTACAGCATACAGGGTTCATGGTGGCAAGAAGATAACAA
GATTTAAATTATGGCCAGTGACTAGTGCTGCAAGAAGAACAACTA
CCTGCATTTAATGGGAAAGCAAAATCTCAGGCTTTGAGGGAAGTT
AACATAGGCTTGATTCTGGGTGGAAGCTGGGTGTGTAGTTATCTG
GAGGCCAGGCTGGAGCTCTCAGCTCACTATGGGTTCATCTTTATT
GTCTCCTTTCATCTCAACAGCTGCACGCTGCCGTCCTCGATGTTG
TGGCGGATCTTGAAGTTCACCTTGATGCCGTTCTTCTGCTTGTCG
GCCATGATATAGACGTTGTGGCTGTTGTAGTTGTACTCCAGCTTG
TGCCCCAGGATGTTGCCGTCCTCCTTGAAGTCGATGCCCTTCAGC
TCGATGCGGTTCACCAGGGTGTCGCCCTCGAACTTCACCTCGGCG
CGGGTCTTGTAGTTGCCGTCGTCCTTGAAGAAGATGGTGCGCTCC
TGGACGTAGCCTTCGGGCATGGCGGACTTGAAGAAGTCGTGCTGC
TTCATGTGGTCGGGGTAGCGGCTGAAGCACTGCACGCCGTAGGTC
AGGGTGGTCACGAGGGTGGGCCAGGGCACGGGCAGCTTGCCGGTG
GTGCAGATGAACTTCAGGGTCAGCTTGCCGTAGGTGGCATCGCCC
TCGCCCTCGCCGGACACGCTGAACTTGTGGCCGTTTACGTCGCCG
TCCAGCTCGACCAGGATGGGCACCACCCCGGTGAACAGCTCCTCG
CCCTTGCTCACCATAGGGCCGGGATTCTCCTCCACGTCACCGCAT
GTTAGAAGACTTCCTCTGCCCTCCATGTTCTCGTAGGAGTCGGCG
TCCTCTTCGTGGTTAGGTCCAGGTTGGCCTCTGATAGACCGCAGC
TGAGGAGCGGCGTACAGAATGCCTCTCATGTCCTCATAGCTGCCG
CTGCCTTGTGGAGGCTTCTCGTGCTTCAGTGTCTCGTATGTCTCT
TGATTCCGGGTGCTCAGGCCGGTGTACACGCCATCAGATTTCTCG
TAGCTGGTGATGGCGGCCTTCCGCACTTGGATCTTCAGCCGTCTG
CAGTACAGGGTGATGACCAGAGACAGCAGCAGGACACCACATGTG
CCAGCCAGAGGGGCCCAAATGTAGATATCCAGGCCTCTGGTATGC
ACAGCTCCGCCTGCAGCAGGTCTACAGGCTTCAGGTCTGAGAGAC
AGAGGCTGGCTGGCGATTGTAGGAGCTGGTGTAGGTGGTCTAGGA
GCGGGTGTTGTTGTAGGCTTGGCGGGCAGAAACACGGGCACGAAG
TGGCTGAAGTACATGATGCTATTGCTCAGGGCTCCGCTTCCTCCG
CCGCCTGATTTGATTTCCAGCTTGGTGCCTCCGCCAAATGTCCAA
GGGCTCTCGTCGTACTGCTGGCAGTAGTAGATGCCGAAGTCCTCG
TACTGCAGGCTGCTGATTGTCAGGGTGTAGTCGGTGCCAGAGCCG
CTGCCAGAAAATCTGCTTGGCACGCCGCTTTCCAGTCTGTTGGCC
CGGTAGATCAGTGTCTTAGGGGCCTTGCCAGGCTTCTGCTGGAAC
CAGCTCAGGTAGCTGTTGATGTCCTGGCTGGCTCTACAGGTGATG
GTCACTCTATCGCCCACAGAGGCAGACAGGCTGCTAGGGCTCTGT
GTCATCTGGATATCAGAGCCACCACCGCCAGATCCACCGCCACCT
GATCCTCCGCCTCCGCTAGAAACTGTCACTGTGGTGCCCTGGCCC
CACACATCGAAGTACCAGTCGTAGCCTCTTCTGGTGCAGAAGTAC
ACGGCGGTATCCTCGGCTCTCAGGCTGTTGATCTGCAGGTAGGCG
GTGTTCTTGCTGTCGTCCAGGCTGAAGGTGAATCTGCCCTTAAAG
CTATCGGCGTAGGTTGGCTCGCCGGTGTGGGTATTGATCCAGCCC
ATCCACTCAAGGCCTTTTCCAGGGGCCTGTCGGACCCAGTTCATG
CCGTAGTTGGTGAAGGTGTAGCCGCTGGCGGCACAGCTGATTCTG
ACAGATCCGCCAGGTTTCACAAGTCCGCCGCCAGACTGAACCAGC
TGGATCTCAGAGATGCTACAGGCCACTGTTCCCAGCAGCAGCAGA
GACTGCAGCCACATTCGAAGCTTGAGCTCGAGATCTGAGTCCGGT
AGCGCTAGCGGATCTGACGGTTCACTAAACCAGCTCTGCTTATAT
AGACCTCCCACCGTACACGCCTACCGCCCATTTGCGTCAATGGGG
CGGAGTTGTTACGACATTTTGGAAAGTCCCGTTGATTTTGGTGCC
AAAACAAACTCCCATTGACGTCAATGGGGTGGAGACTTGGAAATC
CCCGTGAGTCAAACCGCTATCCACGCCCATTGATGTACTGCCAAA
ACCGCATCACCATGGTAATAGCGATGACTAATACGTAGATGTACT
GCCAAGTAGGAAAGTCCCATAAGGTCATGTACTGGGCATAATGCC
AGGCGGGCCATTTACCGTCATTGACGTCAATAGGGGGCGTACTTG
GCATATGATACACTTGATGTACTGCCAAGTGGGCAGTTTACCGTA
AATACTCCACCCATTGACGTCAATGGAAAGTCCCTATTGGCGTTA
CTATGGGAACATACGTCATTATTGACGTCAATGGGCGGGGGTCGT
TGGGCGGTCAGCCAGGCGGGCCATTTACCGTAAGTTATGTAACGC
GGAACTCCATATATGGGCTATGAACTAATGACCCCGTAATTGATT
ACTATTAGCCCGGGGGATCCAGACATGATAAGATACATTGATGAG
TTTGGACAAACCACAACTAGAATGCAGTGAAAAAAATGCTTTATT
TGTGAAATTTGTGATGCTATTGCTTTATTTGTAACCATTATAAGC
TGCAATAAACAAGTTAACAACAACAATTGCATTCATTTTATGTTT
CAGGTTCAGGGGGAGGTGTGGGAGGTTTTTTAAAGCAAGTAAAAC
CTCTACAAATGTGGTATGGCTGATTATGATCCGGCTGCCTCGCGC
GTTTCGGTGATGACGGTGAAAACCTCTGACACATGCAGCTCCCGG
AGACGGTCACAGCTTGTCTGTAAGCGGATGCCGGGAGCAGACAAG
CCCGTCAGGGCGCGTCAGCGGGTGTTGGCGGGTGTCGGGGCGCAG
CCATGAGGTCGATCGACTCTAGAGGATCGATCCCCGCCCCGGACG
AACTAAACCTGACTACGACATCTCTGCCCCTTCTTCGCGGGGCAG
TGCATGTAATCCCTTCAGTTGGTTGGTACAACTTGCCAACTGGGC
CCTGTTCCACATGTGACACGGGGGGGGACCAAACACAAAGGGGTT
CTCTGACTGTAGTTGACATCCTTATAAATGGATGTGCACATTTGC
CAACACTGAGTGGCTTTCATCCTGGAGCAGACTTTGCAGTCTGTG
GACTGCAACACAACATTGCCTTTATGTGTAACTCTTGGCTGAAGC
TCTTACACCAATGCTGGGGGACATGTACCTCCCAGGGGCCCAGGA
AGACTACGGGAGGCTACACCAACGTCAATCAGAGGGGCCTGTGTA
GCTACCGATAAGCGGACCCTCAAGAGGGCATTAGCAATAGTGTTT
ATAAGGCCCCCTTGTTAACCCTAAACGGGTAGCATATGCTTCCCG
GGTAGTAGTATATACTATCCAGACTAACCCTAATTCAATAGCATA
TGTTACCCAACGGGAAGCATATGCTATCGAATTAGGGTTAGTAAA
AGGGTCCTAAGGAACAGCGATATCTCCCACCCCATGAGCTGTCAC
GGTTTTATTTACATGGGGTCAGGATTCCACGAGGGTAGTGAACCA
TTTTAGTCACAAGGGCAGTGGCTGAAGATCAAGGAGCGGGCAGTG
AACTCTCCTGAATCTTCGCCTGCTTCTTCATTCTCCTTCGTTTAG
CTAATAGAATAACTGCTGAGTTGTGAACAGTAAGGTGTATGTGAG
GTGCTCGAAAACAAGGTTTCAGGTGACGCCCCCAGAATAAAATTT
GGACGGGGGGTTCAGTGGTGGCATTGTGCTATGACACCAATATAA
CCCTCACAAACCCCTTGGGCAATAAATACTAGTGTAGGAATGAAA
CATTCTGAATATCTTTAACAATAGAAATCCATGGGGTGGGGACAA
GCCGTAAAGACTGGATGTCCATCTCACACGAATTTATGGCTATGG
GCAACACATAATCCTAGTGCAATATGATACTGGGGTTATTAAGAT
GTGTCCCAGGCAGGGACCAAGACAGGTGAACCATGTTGTTACACT
CTATTTGTAACAAGGGGAAAGAGAGTGGACGCCGACAGCAGCGGA
CTCCACTGGTTGTCTCTAACACCCCCGAAAATTAAACGGGGCTCC
ACGCCAATGGGGCCCATAAACAAAGACAAGTGGCCACTCTTTTTT
TTGAAATTGTGGAGTGGGGGCACGCGTCAGCCCCCACACGCCGCC
CTGCGGTTTTGGACTGTAAAATAAGGGTGTAATAACTTGGCTGAT
TGTAACCCCGCTAACCACTGCGGTCAAACCACTTGCCCACAAAAC
CACTAATGGCACCCCGGGGAATACCTGCATAAGTAGGTGGGCGGG
CCAAGATAGGGGCGCGATTGCTGCGATCTGGAGGACAAATTACAC
ACACTTGCGCCTGAGCGCCAAGCACAGGGTTGTTGGTCCTCATAT
TCACGAGGTCGCTGAGAGCACGGTGGGCTAATGTTGCCATGGGTA
GCATATACTACCCAAATATCTGGATAGCATATGCTATCCTAATCT
ATATCTGGGTAGCATAGGCTATCCTAATCTATATCTGGGTAGCAT
ATGCTATCCTAATCTATATCTGGGTAGTATATGCTATCCTAATTT
ATATCTGGGTAGCATAGGCTATCCTAATCTATATCTGGGTAGCAT
ATGCTATCCTAATCTATATCTGGGTAGTATATGCTATCCTAATCT
GTATCCGGGTAGCATATGCTATCCTAATAGAGATTAGGGTAGTAT
ATGCTATCCTAATTTATATCTGGGTAGCATATACTACCCAAATAT
CTGGATAGCATATGCTATCCTAATCTATATCTGGGTAGCATATGC
TATCCTAATCTATATCTGGGTAGCATAGGCTATCCTAATCTATAT
CTGGGTAGCATATGCTATCCTAATCTATATCTGGGTAGTATATGC
TATCCTAATTTATATCTGGGTAGCATAGGCTATCCTAATCTATAT
CTGGGTAGCATATGCTATCCTAATCTATATCTGGGTAGTATATGC
TATCCTAATCTGTATCCGGGTAGCATATGCTATCCTCATGCATAT
ACAGTCAGCATATGATACCCAGTAGTAGAGTGGGAGTGCTATCCT
TTGCATATGCCGCCACCTCCCAAGGGGGCGTGAATTTTCGCTGCT
TGTCCTTTTCCTGCATGCTGGTTGCTCCCATTCTTAGGTGAATTT
AAGGAGGCCAGGCTAAAGCCGTCGCATGTCTGATTGCTCACCAGG
TAAATGTCGCTAATGTTTTCCAACGCGAGAAGGTGTTGAGCGCGG
AGCTGAGTGACGTGACAACATGGGTATGCCCAATTGCCCCATGTT
GGGAGGACGAAAATGGTGACAAGACAGATGGCCAGAAATACACCA
ACAGCACGCATGATGTCTACTGGGGATTTATTCTTTAGTGCGGGG
GAATACACGGCTTTTAATACGATTGAGGGCGTCTCCTAACAAGTT
ACATCACTCCTGCCCTTCCTCACCCTCATCTCCATCACCTCCTTC
ATCTCCGTCATCTCCGTCATCACCCTCCGCGGCAGCCCCTTCCAC
CATAGGTGGAAACCAGGGAGGCAAATCTACTCCATCGTCAAAGCT
GCACACAGTCACCCTGATATTGCAGGTAGGAGCGGGCTTTGTCAT
AACAAGGTCCTTAATCGCATCCTTCAAAACCTCAGCAAATATATG
AGTTTGTAAAAAGACCATGAAATAACAGACAATGGACTCCCTTAG
CGGGCCAGGTTGTGGGCCGGGTCCAGGGGCCATTCCAAAGGGGAG
ACGACTCAATGGTGTAAGACGACATTGTGGAATAGCAAGGGCAGT
TCCTCGCCTTAGGTTGTAAAGGGAGGTCTTACTACCTCCATATAC
GAACACACCGGCGACCCAAGTTCCTTCGTCGGTAGTCCTTTCTAC
GTGACTCCTAGCCAGGAGAGCTCTTAAACCTTCTGCAATGTTCTC
AAATTTCGGGTTGGAACCTCCTTGACCACGATGCTTTCCAAACCA
CCCTCCTTTTTTGCGCCTGCCTCCATCACCCTGACCCCGGGGTCC
AGTGCTTGGGCCTTCTCCTGGGTCATCTGCGGGGCCCTGCTCTAT
CGCTCCCGGGGGCACGTCAGGCTCACCATCTGGGCCACCTTCTTG
GTGGTATTCAAAATAATCGGCTTCCCCTACAGGGTGGAAAAATGG
CCTTCTACCTGGAGGGGGCCTGCGCGGTGGAGACCCGGATGATGA
TGACTGACTACTGGGACTCCTGGGCCTCTTTTCTCCACGTCCACG
ACCTCTCCCCCTGGCTCTTTCACGACTTCCCCCCCTGGCTCTTTC
ACGTCCTCTACCCCGGCGGCCTCCACTACCTCCTCGACCCCGGCC
TCCACTACCTCCTCGACCCCGGCCTCCACTGCCTCCTCGACCCCG
GCCTCCACCTCCTGCTCCTGCCCCTCCTGCTCCTGCCCCTCCTCC
TGCTCCTGCCCCTCCTGCCCCTCCTGCTCCTGCCCCTCCTGCCCC
TCCTGCTCCTGCCCCTCCTGCCCCTCCTGCTCCTGCCCCTCCTGC
CCCTCCTCCTGCTCCTGCCCCTCCTGCCCCTCCTCCTGCTCCTGC
CCCTCCTGCCCCTCCTGCTCCTGCCCCTCCTGCCCCTCCTGCTCC
TGCCCCTCCTGCCCCTCCTGCTCCTGCCCCTCCTGCTCCTGCCCC
TCCTGCTCCTGCCCCTCCTGCTCCTGCCCCTCCTGCCCCTCCTGC
CCCTCCTCCTGCTCCTGCCCCTCCTGCTCCTGCCCCTCCTGCCCC
TCCTGCCCCTCCTGCTCCTGCCCCTCCTCCTGCTCCTGCCCCTCC
TGCCCCTCCTGCCCCTCCTCCTGCTCCTGCCCCTCCTGCCCCTCC
TCCTGCTCCTGCCCCTCCTCCTGCTCCTGCCCCTCCTGCCCCTCC
TGCCCCTCCTCCTGCTCCTGCCCCTCCTGCCCCTCCTCCTGCTCC
TGCCCCTCCTCCTGCTCCTGCCCCTCCTGCCCCTCCTGCCCCTCC
TCCTGCTCCTGCCCCTCCTCCTGCTCCTGCCCCTCCTGCCCCTCC
TGCCCCTCCTGCCCCTCCTCCTGCTCCTGCCCCTCCTCCTGCTCC
TGCCCCTCCTGCTCCTGCCCCTCCCGCTCCTGCTCCTGCTCCTGT
TCCACCGTGGGTCCCTTTGCAGCCAATGCAACTTGGACGTTTTTG
GGGTCTCCGGACACCATCTCTATGTCTTGGCCCTGATCCTGAGCC
GCCCGGGGCTCCTGGTCTTCCGCCTCCTCGTCCTCGTCCTCTTCC
CCGTCCTCGTCCATGGTTATCACCCCCTCTTCTTTGAGGTCCACT
GCCGCCGGAGCCTTCTGGTCCAGATGTGTCTCCCTTCTCTCCTAG
GCCATTTCCAGGTCCTGTACCTGGCCCCTCGTCAGACATGATTCA
CACTAAAAGAGATCAATAGACATCTTTATTAGACGACGCTCAGTG
AATACAGGGAGTGCAGACTCCTGCCCCCTCCAACAGCCCCCCCAC
CCTCATCCCCTTCATGGTCGCTGTCAGACAGATCCAGGTCTGAAA
ATTCCCCATCCTCCGAACCATCCTCGTCCTCATCACCAATTACTC
GCAGCCCGGAAAACTCCCGCTGAACATCCTCAAGATTTGCGTCCT
GAGCCTCAAGCCAGGCCTCAAATTCCTCGTCCCCCTTTTTGCTGG
ACGGTAGGGATGGGGATTCTCGGGACCCCTCCTCTTCCTCTTCAA
GGTCACCAGACAGAGATGCTACTGGGGCAACGGAAGAAAAGCTGG
GTGCGGCCTGTGAGGATCAGCTTATCGATGATAAGCTGTCAAACA
TGAGAATTCTTGAAGACGAAAGGGCCTCGTGATACGCCTATTTTT
ATAGGTTAATGTCATGATAATAATGGTTTCTTAGACGTCAGGTGG
CACTTTTCGGGGAAATGTGCGCGGAACCCCTATTTGTTTATTTTT
CTAAATACATTCAAATATGTATCCGCTCATGAGACAATAACCCTG
ATAAATGCTTCAATAATATTGAAAAAGGAAGAGTATGAGTATTCA
ACATTTCCGTGTCGCCCTTATTCCCTTTTTTGCGGCATTTTGCCT
TCCTGTTTTTGCTCACCCAGAAACGCTGGTGAAAGTAAAAGATGC
TGAAGATCAGTTGGGTGCACGAGTGGGTTACATCGAACTGGATCT
CAACAGCGGTAAGATCCTTGAGAGTTTTCGCCCCGAAGAACGTTT
TCCAATGATGAGCACTTTTAAAGTTCTGCTATGTGGCGCGGTATT
ATCCCGTGTTGACGCCGGGCAAGAGCAACTCGGTCGCCGCATACA
CTATTCTCAGAATGACTTGGTTGAGTACTCACCAGTCACAGAAAA
GCATCTTACGGATGGCATGACAGTAAGAGAATTATGCAGTGCTGC
CATAACCATGAGTGATAACACTGCGGCCAACTTACTTCTGACAAC
GATCGGAGGACCGAAGGAGCTAACCGCTTTTTTGCACAACATGGG
GGATCATGTAACTCGCCTTGATCGTTGGGAACCGGAGCTGAATGA
AGCCATACCAAACGACGAGCGTGACACCACGATGCCTGCAGCAAT
GGCAACAACGTTGCGCAAACTATTAACTGGCGAACTACTTACTCT
AGCTTCCCGGCAACAATTAATAGACTGGATGGAGGCGGATAAAGT
TGCAGGACCACTTCTGCGCTCGGCCCTTCCGGCTGGCTGGTTTAT
TGCTGATAAATCTGGAGCCGGTGAGCGTGGGTCTCGCGGTATCAT
TGCAGCACTGGGGCCAGATGGTAAGCCCTCCCGTATCGTAGTTAT
CTACACGACGGGGAGTCAGGCAACTATGGATGAACGAAATAGACA
GATCGCTGAGATAGGTGCCTCACTGATTAAGCATTGGTAACTGTC
AGACCAAGTTTACTCATATATACTTTAGATTGATTTAAAACTTCA
TTTTTAATTTAAAAGGATCTAGGTGAAGATCCTTTTTGATAATCT
CATGACCAAAATCCCTTAACGTGAGTTTTCGTTCCACTGAGCGTC
AGACCCCGTAGAAAAGATCAAAGGATCTTCTTGAGATCCTTTTTT
TCTGCGCGTAATCTGCTGCTTGCAAACAAAAAAACCACCGCTACC
AGCGGTGGTTTGTTTGCCGGATCAAGAGCTACCAACTCTTTTTCC
GAAGGTAACTGGCTTCAGCAGAGCGCAGATACCAAATACTGTCCT
TCTAGTGTAGCCGTAGTTAGGCCACCACTTCAAGAACTCTGTAGC
ACCGCCTACATACCTCGCTCTGCTAATCCTGTTACCAGTGGCTGC
TGCCAGTGGCGATAAGTCGTGTCTTACCGGGTTGGACTCAAGACG
ATAGTTACCGGATAAGGCGCAGCGGTCGGGCTGAACGGGGGGTTC
GTGCACACAGCCCAGCTTGGAGCGAACGACCTACACCGAACTGAG
ATACCTACAGCGTGAGCTATGAGAAAGCGCCACGCTTCCCGAAGG
GAGAAAGGCGGACAGGTATCCGGTAAGCGGCAGGGTCGGAACAGG
AGAGCGCACGAGGGAGCTTCCAGGGGGAAACGCCTGGTATCTTTA
TAGTCCTGTCGGGTTTCGCCACCTCTGACTTGAGCGTCGATTTTT
GTGATGCTCGTCAGGGGGGCGGAGCCTATGGAAAAACGCCAGCAA
CGCGGCCTTTTTACGGTTCCTGGCCTTTTGCTGGCCTTTTGCTCA
CATGTTCTTTCCTGCGTTATCCCCTGATTCTGTGGATAACCGTAT
TACCGCCTTTGAGTGAGCTGATACCGCTCGCCGCAGCCGAACGAC
CGAGCGCAGCGAGTCAGTGAGCGAGGAAGCGGAAGAGCGCCTGAT
GCGGTATTTTCTCCTTACGCATCTGTGCGGTATTTCACACCGCAT
ATGGTGCACTCTCAGTACAATCTGCTCTGATGCCGCATAGTTAAG
CCAGCTGTGGAATGTGTGTCAGTTAGGGTGTGGAAAGTCCCCAGG
CTCCCCAGCAGGCAGAAGTATGCAAAGCATGCATCTCAATTAGTC
AGCAACCAGGTGTGGAAAGTCCCCAGGCTCCCCAGCAGGCAGAAG
TATGCAAAGCATGCATCTCAATTAGTCAGCAACCATAGTCCCGCC
CCTAACTCCGCCCATCCCGCCCCTAACTCCGCCCAGTTCCGCCCA
TTCTCCGCCCCATGGCTGACTAATTTTTTTTATTTATGCAGAGGC
CGAGGCCGCCTCGGCCTCTGAGCTATTCCAGAAGTAGTGAGGAGG
CTTTTTTGGAGGCCTAGGCTTTTGCAAAAAGCTTGCATGCCTGCA
GGTCGGCCGCCACGACCGGTGCCGCCACCATCCCCTGACCCACGC
CCCTGACCCCTCACAAGGAGACGACCTTCCATGACCGAGTACAAG
CCCACGGTGCGCCTCGCCACCCGCGACGACGTCCCCCGGGCCGTA
CGCACCCTCGCCGCCGCGTTCGCCGACTACCCCGCCACGCGCCAC
ACCGTCGACCCGGACCGCCACATCGAGCGGGTCACCGAGCTGCAA
GAACTCTTCCTCACGCGCGTCGGGCTCGACATCGGCAAGGTGTGG
GTCGCGGACGACGGCGCCGCGGTGGCGGTCTGGACCACGCCGGAG
AGCGTCGAAGCGGGGGCGGTGTTCGCCGAGATCGGCCCGCGCATG
GCCGAGTTGAGCGGTTCCCGGCTGGCCGCGCAGCAACAGATGGAA
GGCCTCCTGGCGCCGCACCGGCCCAAGGAGCCCGCGTGGTTCCTG
GCCACCGTCGGCGTCTCGCCCGACCACCAGGGCAAGGGTCTGGGC
AGCGCCGTCGTGCTCCCCGGAGTGGAGGCGGCCGAGCGCGCCGGG
GTGCCCGCCTTCCTGGAGACCTCCGCGCCCCGCAACCTCCCCTTC
TACGAGCGGCTCGGCTTCACCGTCACCGCCGACGTCGAGGTGCCC
GAAGGACCGCGCACCTGGTGCATGACCCGCAAGCCCGGTGCCTGA
CGCCCGCCCCACGACCCGCAGCGCCCGACCGAAAGGAGCGCACGA
CCCCATGGCTCCGACCGAAGCCGACCCGGGCGGCCCCGCCGACCC
CGCACCCGCCCCCGAGGCCCACCGACTCTAGAGGATCATAATCAG
CCATACCACATTTGTAGAGGTTTTACTTGCTTTAAAAAACCTCCC
ACACCTCCCCCTGAACCTGAAACATAAAATGAATGCAATTGTTGT
TGTTAACTTGTTTATTGCAGCTTATAATGGTTACAAATAAAGCAA
TAGCATCACAAATTTCACAAATAAAGCATTTTTTTCACTGCATTC
TAGTTGTGGTTTGTCCAAACTCATCAATGTATCTTATCATGTCTG
GATCACTCGCCGATAGTGGAAACCGACGCCCCAGCACTCGTCCGA
GGGCAAAGGAATAGGGGAGATGGGGGAGGCTAACTGAAACACGGA
AGGAGACAATACCGGAAGGAACCCGCGCTATGACGGCAATAAAAA
GACAGAATAAAACGCACGGGTGTTGGGTCGTTTGTTCATAAACGC
GGGGTTCGGTCCCAGGGCTGGCACTCTGTCGATACCCCACCGAGA
CCCCATTGGGGCCAATACGCCCGCGTTTCTTCCTTTTCCCCACCC
CACCCCCCAAGTTCGGGTGAAGGCCCAGGGCTCGCAGCCAACGTC
GGGGCGGCAGGCCCTGCCATAGCCACTGGCCCCGTGGGTTAGGGA
CGGGGTCCCCCATGGGGAATGGTTTATGGTTCGTGGGGGTTATTA
TTTTGGGCGTTGCGTGGGGTCTGGTCCACGACTGGACTGAGCAGA
CAGACCCATGGTTTTTGGATGGCCTGGGCATGGACCGCATGTACT
GGCGCGACACGAACACCGGGCGTCTGTGGCTGCCAAACACCCCCG
ACCCCCAAAAACCACCGCGCGGATTTCTGGCGTGCCAAGCTAGTC
GACCAATTCTCATGTTTGACAGCTTATCATCGCAGATCCGGGCAA
CGTTGTTGCATTGCTGCAGGCGCAGAACTGGTAGGTATGGAAGAT
CTCTAGAAGCTGGGTACCAGCTGCTAGCAAGCTTGCTAGCGGCCG
GCTCGAGTTTACTCCCTATCAGTGATAGAGAACGTATGTCGAGTT
TACTCCCTATCAGTGATAGAGAACGATGTCGAGTTTACTCCCTAT
CAGTGATAGAGAACGTATGTCGAGTTTACTCCCTATCAGTGATAG
AGAACGTATGTCGAGTTTACTCCCTATCAGTGATAGAGAACGTAT
GTCGAGTTTATCCCTATCAGTGATAGAGAACGTATGTCGAGTTTA
CTCCCTATCAGTGATAGAGAACGTATGTCGAGGTAGGCGTGTACG
GTGGGAGGCCTATATAAGCAGAGCTCGTTTAGTGAACCGTCAGAT
CGCCG (SEQ ID NO: 40)
LINE-1 plasmid Her2-Cd3z-T2A GFP intron
(SEQ ID NO: 41)
CGGCCGCGGGGGGAGGAGCCAAGATGGCCGAATAGGAACAGCTCC
GGTCTACAGCTCCCAGCGTGAGCGACGCAGAAGACGGTGATTTCT
GCATTTCCATCTGAGGTACCGGGTTCATCTCACTAGGGAGTGCCA
GACAGTGGGCGCAGGCCAGTGTGTGTGCGCACCGTGCGCGAGCCG
AAGCAGGGCGAGGCATTGCCTCACCTGGGAAGCGCAAGGGGTCAG
GGAGTTCCCTTTCCGAGTCAAAGAAAGGGGTGACGGACGCACCTG
GAAAATCGGGTCACTCCCACCCGAATATTGCGCTTTTCAGACCGG
CTTAAGAAACGGCGCACCACGAGACTATATCCCACACCTGGCTCG
GAGGGTCCTACGCCCACGGAATCTCGCTGATTGCTAGCACAGCAG
TCTGAGATCAAACTGCAAGGCGGCAACGAGGCTGGGGGAGGGGCG
CCCGCCATTGCCCAGGCTTGCTTAGGTAAACAAAGCAGCAGGGAA
GCTCGAACTGGGTGGAGCCCACCACAGCTCAAGGAGGCCTGCCTG
CCTCTGTAGGCTCCACCTCTGGGGGCAGGGCACAGACAAACAAAA
AGACAGCAGTAACCTCTGCAGACTTAAGTGTCCCTGTCTGACAGC
TTTGAAGAGAGCAGTGGTTCTCCCAGCACGCAGCTGGAGATCTGA
GAACGGGCAGACTGCCTCCTCAAGTGGGTCCCTGACCCCTGACCC
CCGAGCAGCCTAACTGGGAGGCACCCCCCAGCAGGGGCACACTGA
CACCTCACACGGCAGGGTATTCCAACAGACCTGCAGCTGAGGGTC
CTGTCTGTTAGAAGGAAAACTAACAACCAGAAAGGACATCTACAC
CGAAAACCCATCTGTACATCACCATCATCAAAGACCAAAAGTAGA
TAAAACCACAAAGATGGGGAAAAAACAGAACAGAAAAACTGGAAA
CTCTAAAACGCAGAGCGCCTCTCCTCCTCCAAAGGAACGCAGTTC
CTCACCAGCAACAGAACAAAGCTGGATGGAGAATGATTTTGATGA
GCTGAGAGAAGAAGGCTTCAGACGATCAAATTACTCTGAGCTACG
GGAGGACATTCAAACCAAAGGCAAAGAAGTTGAAAACTTTGAAAA
AAATTTAGAAGAATGTATAACTAGAATAACCAATACAGAGAAGTG
CTTAAAGGAGCTGATGGAGCTGAAAACCAAGGCTCGAGAACTACG
TGAAGAATGCAGAAGCCTCAGGAGCCGATGCGATCAACTGGAAGA
AAGGGTATCAGCAATGGAAGATGAAATGAATGAAATGAAGCGAGA
AGGGAAGTTTAGAGAAAAAAGAATAAAAAGAAATGAGCAAAGCCT
CCAAGAAATATGGGACTATGTGAAAAGACCAAATCTACGTCTGAT
TGGTGTACCTGAAAGTGATGTGGAGAATGGAACCAAGTTGGAAAA
CACTCTGCAGGATATTATCCAGGAGAACTTCCCCAATCTAGCAAG
GCAGGCCAACGTTCAGATTCAGGAAATACAGAGAACGCCACAAAG
ATACTCCTCGAGAAGAGCAACTCCAAGACACATAATTGTCAGATT
CACCAAAGTTGAAATGAAGGAAAAAATGTTAAGGGCAGCCAGAGA
GAAAGGTCGGGTTACCCTCAAAGGAAAGCCCATCAGACTAACAGC
GGATCTCTCGGCAGAAACCCTACAAGCCAGAAGAGAGTGGGGGCC
AATATTCAACATTCTTAAAGAAAAGAATTTTCAACCCAGAATTTC
ATATCCAGCCAAACTAAGCTTCATAAGTGAAGGAGAAATAAAATA
CTTTATAGACAAGCAAATGTTGAGAGATTTTGTCACCACCAGGCC
TGCCCTAAAAGAGCTCCTGAAGGAAGCGCTAAACATGGAAAGGAA
CAACCGGTACCAGCCGCTGCAAAATCATGCCAAAATGTAAAGACC
ATCAAGACTAGGAAGAAACTGCATCAACTAATGAGCAAAATCACC
AGCTAACATCATAATGACAGGATCAACTTCACACATAACAATATT
AACTTTAAATATAAATGGACTAAATTCTGCAATTAAAAGACACAG
ACTGGCAAGTTGGATAAAGAGTCAAGACCCATCAGTGTGCTGTAT
TCAGGAAACCCATCTCACGTGCAGAGACACACATAGGCTCAAAAT
AAAAGGATGGAGGAAGATCTACCAAGCCAATGGAAAACAAAAAAA
GGCAGGGGTTGCAATCCTAGTCTCTGATAAAACAGACTTTAAACC
AACAAAGATCAAAAGAGACAAAGAAGGCCATTACATAATGGTAAA
GGGATCAATTCAACAAGAGGAGCTAACTATCCTAAATATTTATGC
ACCCAATACAGGAGCACCCAGATTCATAAAGCAAGTCCTCAGTGA
CCTACAAAGAGACTTAGACTCCCACACATTAATAATGGGAGACTT
TAACACCCCACTGTCAACATTAGACAGATCAACGAGACAGAAAGT
CAACAAGGATACCCAGGAATTGAACTCAGCTCTGCACCAAGCAGA
CCTAATAGACATCTACAGAACTCTCCACCCCAAATCAACAGAATA
TACATTTTTTTCAGCACCACACCACACCTATTCCAAAATTGACCA
CATAGTTGGAAGTAAAGCTCTCCTCAGCAAATGTAAAAGAACAGA
AATTATAACAAACTATCTCTCAGACCACAGTGCAATCAAACTAGA
ACTCAGGATTAAGAATCTCACTCAAAGCCGCTCAACTACATGGAA
ACTGAACAACCTGCTCCTGAATGACTACTGGGTACATAACGAAAT
GAAGGCAGAAATAAAGATGTTCTTTGAAACCAACGAGAACAAAGA
CACCACATACCAGAATCTCTGGGACGCATTCAAAGCAGTGTGTAG
AGGGAAATTTATAGCACTAAATGCCTACAAGAGAAAGCAGGAAAG
ATCCAAAATTGACACCCTAACATCACAATTAAAAGAACTAGAAAA
GCAAGAGCAAACACATTCAAAAGCTAGCAGAAGGCAAGAAATAAC
TAAAATCAGAGCAGAACTGAAGGAAATAGAGACACAAAAAACCCT
TCAAAAAATCAATGAATCCAGGAGCTGGTTTTTTGAAAGGATCAA
CAAAATTGATAGACCGCTAGCAAGACTAATAAAGAAAAAAAGAGA
GAAGAATCAAATAGACACAATAAAAAATGATAAAGGGGATATCAC
CACCGATCCCACAGAAATACAAACTACCATCAGAGAATACTACAA
ACACCTCTACGCAAATAAACTAGAAAATCTAGAAGAAATGGATAC
ATTCCTCGACACATACACTCTCCCAAGACTAAACCAGGAAGAAGT
TGAATCTCTGAATCGACCAATAACAGGCTCTGAAATTGTGGCAAT
AATCAATAGTTTACCAACCAAAAAGAGTCCAGGACCAGATGGATT
CACAGCCGAATTCTACCAGAGGTACAAGGAGGAACTGGTACCATT
CCTTCTGAAACTATTCCAATCAATAGAAAAAGAGGGAATCCTCCC
TAACTCATTTTATGAGGCCAGCATCATTCTGATACCAAAGCCGGG
CAGAGACACAACCAAAAAAGAGAATTTTAGACCAATATCCTTGAT
GAACATTGATGCAAAAATCCTCAATAAAATACTGGCAAACCGAAT
CCAGCAGCACATCAAAAAGCTTATCCACCATGATCAAGTGGGCTT
CATCCCTGGGATGCAAGGCTGGTTCAATATACGCAAATCAATAAA
TGTAATCCAGCATATAAACAGAGCCAAAGACAAAAACCACATGAT
TATCTCAATAGATGCAGAAAAAGCCTTTGACAAAATTCAACAACC
CTTCATGCTAAAAACTCTCAATAAATTAGGTATTGATGGGACGTA
TTTCAAAATAATAAGAGCTATCTATGACAAACCCACAGCCAATAT
CATACTGAATGGGCAAAAACTGGAAGCATTCCCTTTGAAAACCGG
CACAAGACAGGGATGCCCTCTCTCACCGCTCCTATTCAACATAGT
GTTGGAAGTTCTGGCCAGGGCAATCAGGCAGGAGAAGGAAATAAA
GGGTATTCAATTAGGAAAAGAGGAAGTCAAATTGTCCCTGTTTGC
AGACGACATGATTGTTTATCTAGAAAACCCCATCGTCTCAGCCCA
AAATCTCCTTAAGCTGATAAGCAACTTCAGCAAAGTCTCAGGATA
CAAAATCAATGTACAAAAATCACAAGCATTCTTATACACCAACAA
CAGACAAACAGAGAGCCAAATCATGGGTGAACTCCCATTCACAAT
TGCTTCAAAGAGAATAAAATACCTAGGAATCCAACTTACAAGGGA
TGTGAAGGACCTCTTCAAGGAGAACTACAAACCACTGCTCAAGGA
AATAAAAGAGGAGACAAACAAATGGAAGAACATTCCATGCTCATG
GGTAGGAAGAATCAATATCGTGAAAATGGCCATACTGCCCAAGGT
AATTTACAGATTCAATGCCATCCCCATCAAGCTACCAATGACTTT
CTTCACAGAATTGGAAAAAACTACTTTAAAGTTCATATGGAACCA
AAAAAGAGCCCGCATTGCCAAGTCAATCCTAAGCCAAAAGAACAA
AGCTGGAGGCATCACACTACCTGACTTCAAACTATACTACAAGGC
TACAGTAACCAAAACAGCATGGTACTGGTACCAAAACAGAGATAT
AGATCAATGGAACAGAACAGAGCCCTCAGAAATAATGCCGCATAT
CTACAACTATCTGATCTTTGACAAACCTGAGAAAAACAAGCAATG
GGGAAAGGATTCCCTATTTAATAAATGGTGCTGGGAAAACTGGCT
AGCCATATGTAGAAAGCTGAAACTGGATCCCTTCCTTACACCTTA
TACAAAAATCAATTCAAGATGGATTAAAGATTTAAACGTTAAACC
TAAAACCATAAAAACCCTAGAAGAAAACCTAGGCATTACCATTCA
GGACATAGGCGTGGGCAAGGACTTCATGTCCAAAACACCAAAAGC
AATGGCAACAAAAGACAAAATTGACAAATGGGATCTAATTAAACT
AAAGAGCTTCTGCACAGCAAAAGAAACTACCATCAGAGTGAACAG
GCAACCTACAACATGGGAGAAAATTTTTGCAACCTACTCATCTGA
CAAAGGGCTAATATCCAGAATCTACAATGAACTCAAACAAATTTA
CAAGAAAAAAACAAACAACCCCATCAAAAAGTGGGCGAAGGACAT
GAACAGACACTTCTCAAAAGAAGACATTTATGCAGCCAAAAAACA
CATGAAGAAATGCTCATCATCACTGGCCATCAGAGAAATGCAAAT
CAAAACCACTATGAGATATCATCTCACACCAGTTAGAATGGCAAT
CATTAAAAAGTCAGGAAACAACAGGTGCTGGAGAGGATGCGGAGA
AATAGGAACACTTTTACACTGTTGGTGGGACTGTAAACTAGTTCA
ACCATTGTGGAAGTCAGTGTGGCGATTCCTCAGGGATCTAGAACT
AGAAATACCATTTGACCCAGCCATCCCATTACTGGGTATATACCC
AAATGAGTATAAATCATGCTGCTATAAAGACACATGCACACGTAT
GTTTATTGCGGCACTATTCACAATAGCAAAGACTTGGAACCAACC
CAAATGTCCAACAATGATAGACTGGATTAAGAAAATGTGGCACAT
ATACACCATGGAATACTATGCAGCCATAAAAAATGATGAGTTCAT
ATCCTTTGTAGGGACATGGATGAAATTGGAAACCATCATTCTCAG
TAAACTATCGCAAGAACAAAAAACCAAACACCGCATATTCTCACT
CATAGGTGGGAATTGAACAATGAGATCACATGGACACAGGAAGGG
GAATATCACACTCTGGGGACTGTGGTGGGGTCGGGGGAGGGGGGA
GGGATAGCATTGGGAGATATACCTAATGCTAGATGACACATTAGT
GGGTGCAGCGCACCAGCATGGCACATGTATACGGATCCGAATTCT
CGACGGATCGATCCGAACAAACGACCCAACACCCGTGCGTTTTAT
TCTGTCTTTTTATTGCCGATCCCCTCAGAAGAACTCGTCAAGAAG
GCGATAGAAGGCGATGCGCTGCGAATCGGGAGCGGCGATACCGTA
AAGCACGAGGAAGCGGTCAGCCCATTCGCCGCCAAGCTCTTCAGC
AATATCACGGGTAGCCAACGCTATGTCCTGATAGCGGTCGGCCGC
TTTACTTGTACAGCTCGTCCATGCCGAGAGTGATCCCGGCGGCGG
TCACGAACTCCAGCAGGACCATGTGATCGCGCTTCTCGTTGGGGT
CTTTGCTCAGGGCGGACTGGGTGCTCAGGTAGTGGTTGTCGGGCA
GCAGCACGGGGCCGTCGCCGATGGGGGTGTTCTGCTGGTAGTGGT
CGGCCAGGTGAGTCCAGGAGATGTTTCAGCACTGTTGCCTTTAGT
CTCGAGGCAACTTAGACAACTGAGTATTGATCTGAGCACAGCAGG
GTGTGAGCTGTTTGAAGATACTGGGGTTGGGGGTGAAGAAACTGC
AGAGGACTAACTGGGCTGAGACCCAGTGGCAATGTTTTAGGGCCT
AAGGAATGCCTCTGAAAATCTAGATGGACAACTTTGACTTTGAGA
AAAGAGAGGTGGAAATGAGGAAAATGACTTTTCTTTATTAGATTT
CGGTAGAAAGAACTTTCATCTTTCCCCTATTTTTGTTATTCGTTT
TAAAACATCTATCTGGAGGCAGGACAAGTATGGTCATTAAAAAGA
TGCAGGCAGAAGGCATATATTGGCTCAGTCAAAGTGGGGAACTTT
GGTGGCCAAACATACATTGCTAAGGCTATTCCTATATCAGCTGGA
CACATATAAAATGCTGCTAATGCTTCATTACAAACTTATATCCTT
TAATTCCAGATGGGGGCAAAGTATGTCCAGGGGTGAGGAACAATT
GAAACATTTGGGCTGGAGTAGATTTTGAAAGTCAGCTCTGTGTGT
GTGTGTGTGTGTGTGTGTGTGAGAGCGTGTGTTTCTTTTAACGTT
TTCAGCCTACAGCATACAGGGTTCATGGTGGCAAGAAGATAACAA
GATTTAAATTATGGCCAGTGACTAGTGCTGCAAGAAGAACAACTA
CCTGCATTTAATGGGAAAGCAAAATCTCAGGCTTTGAGGGAAGTT
AACATAGGCTTGATTCTGGGTGGAAGCTGGGTGTGTAGTTATCTG
GAGGCCAGGCTGGAGCTCTCAGCTCACTATGGGTTCATCTTTATT
GTCTCCTTTCATCTCAACAGCTGCACGCTGCCGTCCTCGATGTTG
TGGCGGATCTTGAAGTTCACCTTGATGCCGTTCTTCTGCTTGTCG
GCCATGATATAGACGTTGTGGCTGTTGTAGTTGTACTCCAGCTTG
TGCCCCAGGATGTTGCCGTCCTCCTTGAAGTCGATGCCCTTCAGC
TCGATGCGGTTCACCAGGGTGTCGCCCTCGAACTTCACCTCGGCG
CGGGTCTTGTAGTTGCCGTCGTCCTTGAAGAAGATGGTGCGCTCC
TGGACGTAGCCTTCGGGCATGGCGGACTTGAAGAATAGTGAACCG
TCAGATCGCCGGTCGTGCTGCTTCATGTGGTCGGGGTAGCGGCTG
AAGCACTGCACGCCGTAGGTCAGGGTGGTCACGAGGGTGGGCCAG
GGCACGGGCAGCTTGCCGGTGGTGCAGATGAACTTCAGGGTCAGC
TTGCCGTAGGTGGCATCGCCCTCGCCCTCGCCGGACACGCTGAAC
TTGTGGCCGTTTACGTCGCCGTCCAGCTCGACCAGGATGGGCACC
ACCCCGGTGAACAGCTCCTCGCCCTTGCTCACCATAGGGCCGGGA
TTCTCCTCCACGTCACCGCATGTTAGAAGACTTCCTCTGCCCTCT
CTTGGAGGCAGGGCCTGCATGTGCAGGGCATCGTAGGTATCCTTG
GTGGCTGTGCTCAGTCCCTGGTACAGTCCATCGTGGCCCTTGCCT
CTTCTTCTCTCGCCCTTCATGCCGATCTCGCTGTAGGCCTCGGCC
ATCTTGTCTTTCTGCAGCTCATTATACAGGCCCTCTTGAGGATTC
TTTCTCCGCTGGGGCTTGCCGCCCATCTCAGGATCTCTGCCTCTC
CGCTTATCCAGCACGTCGTACTCTTCTCTTCTCCCCAGGTTCAGC
TCGTTGTACAGCTGATTCTGGCCCTGCTGGTAAGCAGGAGCGTCG
GCGGATCTGCTGAACTTCACTCTGCAGTACAGGGTGATGACCAGA
GAGAGCAGCAGAACGCCACATGTGCCAGCCAGAGGGGCCCAAATG
TAGATATCCAGGCCTCTGGTATGCACAGCTCCGCCAGCTGCAGGT
CTACAGGCTTCAGGTCTGAGAGACAGAGGCTGGCTGGCGATTGTA
GGAGCTGGTGTAGGTGGTCTAGGAGCGGGTGTTGTTGTAGGCTTG
GCGGGCAGAAACACGGGCACGAAGTGGCTGAAGTACATGATGCTA
TTGCTCAGGGCTCCGCTTCCTCCGCCTCCGCTAGAAGAAACTGTG
ACCAGGGTGCCCTGTCCCCAAACATCCATGGCGTAGAAGCCGTCG
CCTCCCCATCTAGAACAGTAGTACACGGCGGTGTCCTCGGCTCTC
AGGCTGTTCATCTGCAGGTAGGCGGTGTTCTTGCTGGTGTCGGCG
CTGATGGTGAATCTGCCCTTCACGCTATCGGCGTATCTGGTGTAG
CCGTTGGTGGGGTAGATTCTGGCGACCCATTCAAGTCCCTTTCCA
GGGGCCTGTCGGACCCAGTGGATGTAGGTGTCCTTGATGTTGAAG
CCGCTGGCGGCACAAGACAGTCTCAGAGAGCCGCCAGGCTGAACA
AGTCCTCCGCCAGATTCAACCAGCTGCACCTCAGATCCTTCGCCA
GATCCAGGCTTTCCAGAGCCGCTGGTGCTGCCTGTTCTCTTGATT
TCCACCTTGGTGCCCTGGCCAAAGGTTGGAGGTGTGGTGTAGTGC
TGCTGGCAGTAGTAGGTGGCGAAGTCCTCAGGCTGCAGGCTAGAG
ATGGTCAGGGTGAAGTCGGTGCCAGATCTGCTGCCGCTGAATCTG
CTTGGCACGCCGCTGTACAGAAAGCTGGCGCTGTAGATCAGCAGC
TTAGGGGCTTTTCCAGGCTTCTGCTGATACCAGGCCACGGCGGTA
TTCACATCCTGGCTGGCTCTACAGGTGATGGTCACTCTATCGCCC
ACAGAGGCAGACAGGCTGCTAGGGCTCTGTGTCATCTGGATGTCG
CTGATGCTGCAGGCCACTGTTCCCAGCAGCAGCAGAGACTGCAGC
CACATTCGAAGCTTGAGCTCGAGATCTGAGTCCGGTAGCGCTAGC
GGATCTGACGGTTCACTAAACCAGCTCTGCTTATATAGACCTCCC
ACCGTACACGCCTACCGCCCATTTGCGTCAATGGGGCGGAGTTGT
TACGACATTTTGGAAAGTCCCGTTGATTTTGGTGCCAAAACAAAC
TCCCATTGACGTCAATGGGGTGGAGACTTGGAAATCCCCGTGAGT
CAAACCGCTATCCACGCCCATTGATGTACTGCCAAAACCGCATCA
CCATGGTAATAGCGATGACTAATACGTAGATGTACTGCCAAGTAG
GAAAGTCCCATAAGGTCATGTACTGGGCATAATGCCAGGCGGGCC
ATTTACCGTCATTGACGTCAATAGGGGGCGTACTTGGCATATGAT
ACACTTGATGTACTGCCAAGTGGGCAGTTTACCGTAAATACTCCA
CCCATTGACGTCAATGGAAAGTCCCTATTGGCGTTACTATGGGAA
CATACGTCATTATTGACGTCAATGGGCGGGGGTCGTTGGGCGGTC
AGCCAGGCGGGCCATTTACCGTAAGTTATGTAACGCGGAACTCCA
TATATGGGCTATGAACTAATGACCCCGTAATTGATTACTATTAGC
CCGGGGGATCCAGACATGATAAGATACATTGATGAGTTTGGACAA
ACCACAACTAGAATGCAGTGAAAAAAATGCTTTATTTGTGAAATT
TGTGATGCTATTGCTTTATTTGTAACCATTATAAGCTGCAATAAA
CAAGTTAACAACAACAATTGCATTCATTTTATGTTTCAGGTTCAG
GGGGAGGTGTGGGAGGTTTTTTAAAGCAAGTAAAACCTCTACAAA
TGTGGTATGGCTGATTATGATCCGGCTGCCTCGCGCGTTTCGGTG
ATGACGGTGAAAACCTCTGACACATGCAGCTCCCGGAGACGGTCA
CAGCTTGTCTGTAAGCGGATGCCGGGAGCAGACAAGCCCGTCAGG
GCGCGTCAGCGGGTGTTGGCGGGTGTCGGGGCGCAGCCATGAGGT
CGATCGACTCTAGAGGATCGATCCCCGCCCCGGACGAACTAAACC
TGACTACGACATCTCTGCCCCTTCTTCGCGGGGCAGTGCATGTAA
TCCCTTCAGTTGGTTGGTACAACTTGCCAACTGGGCCCTGTTCCA
CATGTGACACGGGGGGGGACCAAACACAAAGGGGTTCTCTGACTG
TAGTTGACATCCTTATAAATGGATGTGCACATTTGCCAACACTGA
GTGGCTTTCATCCTGGAGCAGACTTTGCAGTCTGTGGACTGCAAC
ACAACATTGCCTTTATGTGTAACTCTTGGCTGAAGCTCTTACACC
AATGCTGGGGGACATGTACCTCCCAGGGGCCCAGGAAGACTACGG
GAGGCTACACCAACGTCAATCAGAGGGGCCTGTGTAGCTACCGAT
AAGCGGACCCTCAAGAGGGCATTAGCAATAGTGTTTATAAGGCCC
CCTTGTTAACCCTAAACGGGTAGCATATGCTTCCCGGGTAGTAGT
ATATACTATCCAGACTAACCCTAATTCAATAGCATATGTTACCCA
ACGGGAAGCATATGCTATCGAATTAGGGTTAGTAAAAGGGTCCTA
AGGAACAGCGATATCTCCCACCCCATGAGCTGTCACGGTTTTATT
TACATGGGGTCAGGATTCCACGAGGGTAGTGAACCATTTTAGTCA
CAAGGGCAGTGGCTGAAGATCAAGGAGCGGGCAGTGAACTCTCCT
GAATCTTCGCCTGCTTCTTCATTCTCCTTCGTTTAGCTAATAGAA
TAACTGCTGAGTTGTGAACAGTAAGGTGTATGTGAGGTGCTCGAA
AACAAGGTTTCAGGTGACGCCCCCAGAATAAAATTTGGACGGGGG
GTTCAGTGGTGGCATTGTGCTATGACACCAATATAACCCTCACAA
ACCCCTTGGGCAATAAATACTAGTGTAGGAATGAAACATTCTGAA
TATCTTTAACAATAGAAATCCATGGGGTGGGGACAAGCCGTAAAG
ACTGGATGTCCATCTCACACGAATTTATGGCTATGGGCAACACAT
AATCCTAGTGCAATATGATACTGGGGTTATTAAGATGTGTCCCAG
GCAGGGACCAAGACAGGTGAACCATGTTGTTACACTCTATTTGTA
ACAAGGGGAAAGAGAGTGGACGCCGACAGCAGCGGACTCCACTGG
TTGTCTCTAACACCCCCGAAAATTAAACGGGGCTCCACGCCAATG
GGGCCCATAAACAAAGACAAGTGGCCACTCTTTTTTTTGAAATTG
TGGAGTGGGGGCACGCGTCAGCCCCCACACGCCGCCCTGCGGTTT
TGGACTGTAAAATAAGGGTGTAATAACTTGGCTGATTGTAACCCC
GCTAACCACTGCGGTCAAACCACTTGCCCACAAAACCACTAATGG
CACCCCGGGGAATACCTGCATAAGTAGGTGGGCGGGCCAAGATAG
GGGCGCGATTGCTGCGATCTGGAGGACAAATTACACACACTTGCG
CCTGAGCGCCAAGCACAGGGTTGTTGGTCCTCATATTCACGAGGT
CGCTGAGAGCACGGTGGGCTAATGTTGCCATGGGTAGCATATACT
ACCCAAATATCTGGATAGCATATGCTATCCTAATCTATATCTGGG
TAGCATAGGCTATCCTAATCTATATCTGGGTAGCATATGCTATCC
TAATCTATATCTGGGTAGTATATGCTATCCTAATTTATATCTGGG
TAGCATAGGCTATCCTAATCTATATCTGGGTAGCATATGCTATCC
TAATCTATATCTGGGTAGTATATGCTATCCTAATCTGTATCCGGG
TAGCATATGCTATCCTAATAGAGATTAGGGTAGTATATGCTATCC
TAATTTATATCTGGGTAGCATATACTACCCAAATATCTGGATAGC
ATATGCTATCCTAATCTATATCTGGGTAGCATATGCTATCCTAAT
CTATATCTGGGTAGCATAGGCTATCCTAATCTATATCTGGGTAGC
ATATGCTATCCTAATCTATATCTGGGTAGTATATGCTATCCTAAT
TTATATCTGGGTAGCATAGGCTATCCTAATCTATATCTGGGTAGC
ATATGCTATCCTAATCTATATCTGGGTAGTATATGCTATCCTAAT
CTGTATCCGGGTAGCATATGCTATCCTCATGCATATACAGTCAGC
ATATGATACCCAGTAGTAGAGTGGGAGTGCTATCCTTTGCATATG
CCGCCACCTCCCAAGGGGGCGTGAATTTTCGCTGCTTGTCCTTTT
CCTGCATGCTGGTTGCTCCCATTCTTAGGTGAATTTAAGGAGGCC
AGGCTAAAGCCGTCGCATGTCTGATTGCTCACCAGGTAAATGTCG
CTAATGTTTTCCAACGCGAGAAGGTGTTGAGCGCGGAGCTGAGTG
ACGTGACAACATGGGTATGCCCAATTGCCCCATGTTGGGAGGACG
AAAATGGTGACAAGACAGATGGCCAGAAATACACCAACAGCACGC
ATGATGTCTACTGGGGATTTATTCTTTAGTGCGGGGGAATACACG
GCTTTTAATACGATTGAGGGCGTCTCCTAACAAGTTACATCACTC
CTGCCCTTCCTCACCCTCATCTCCATCACCTCCTTCATCTCCGTC
ATCTCCGTCATCACCCTCCGCGGCAGCCCCTTCCACCATAGGTGG
AAACCAGGGAGGCAAATCTACTCCATCGTCAAAGCTGCACACAGT
CACCCTGATATTGCAGGTAGGAGCGGGCTTTGTCATAACAAGGTC
CTTAATCGCATCCTTCAAAACCTCAGCAAATATATGAGTTTGTAA
AAAGACCATGAAATAACAGACAATGGACTCCCTTAGCGGGCCAGG
TTGTGGGCCGGGTCCAGGGGCCATTCCAAAGGGGAGACGACTCAA
TGGTGTAAGACGACATTGTGGAATAGCAAGGGCAGTTCCTCGCCT
TAGGTTGTAAAGGGAGGTCTTACTACCTCCATATACGAACACACC
GGCGACCCAAGTTCCTTCGTCGGTAGTCCTTTCTACGTGACTCCT
AGCCAGGAGAGCTCTTAAACCTTCTGCAATGTTCTCAAATTTCGG
GTTGGAACCTCCTTGACCACGATGCTTTCCAAACCACCCTCCTTT
TTTGCGCCTGCCTCCATCACCCTGACCCCGGGGTCCAGTGCTTGG
GCCTTCTCCTGGGTCATCTGCGGGGCCCTGCTCTATCGCTCCCGG
GGGCACGTCAGGCTCACCATCTGGGCCACCTTCTTGGTGGTATTC
AAAATAATCGGCTTCCCCTACAGGGTGGAAAAATGGCCTTCTACC
TGGAGGGGGCCTGCGCGGTGGAGACCCGGATGATGATGACTGACT
ACTGGGACTCCTGGGCCTCTTTTCTCCACGTCCACGACCTCTCCC
CCTGGCTCTTTCACGACTTCCCCCCCTGGCTCTTTCACGTCCTCT
ACCCCGGCGGCCTCCACTACCTCCTCGACCCCGGCCTCCACTACC
TCCTCGACCCCGGCCTCCACTGCCTCCTCGACCCCGGCCTCCACC
TCCTGCTCCTGCCCCTCCTGCTCCTGCCCCTCCTCCTGCTCCTGC
CCCTCCTGCCCCTCCTGCTCCTGCCCCTCCTGCCCCTCCTGCTCC
TGCCCCTCCTGCCCCTCCTGCTCCTGCCCCTCCTGCCCCTCCTCC
TGCTCCTGCCCCTCCTGCCCCTCCTCCTGCTCCTGCCCCTCCTGC
CCCTCCTGCTCCTGCCCCTCCTGCCCCTCCTGCTCCTGCCCCTCC
TGCCCCTCCTGCTCCTGCCCCTCCTGCTCCTGCCCCTCCTGCTCC
TGCCCCTCCTGCTCCTGCCCCTCCTGCCCCTCCTGCCCCTCCTCC
TGCTCCTGCCCCTCCTGCTCCTGCCCCTCCTGCCCCTCCTGCCCC
TCCTGCTCCTGCCCCTCCTCCTGCTCCTGCCCCTCCTGCCCCTCC
TGCCCCTCCTCCTGCTCCTGCCCCTCCTGCCCCTCCTCCTGCTCC
TGCCCCTCCTCCTGCTCCTGCCCCTCCTGCCCCTCCTGCCCCTCC
TCCTGCTCCTGCCCCTCCTGCCCCTCCTCCTGCTCCTGCCCCTCC
TCCTGCTCCTGCCCCTCCTGCCCCTCCTGCCCCTCCTCCTGCTCC
TGCCCCTCCTCCTGCTCCTGCCCCTCCTGCCCCTCCTGCCCCTCC
TGCCCCTCCTCCTGCTCCTGCCCCTCCTCCTGCTCCTGCCCCTCC
TGCTCCTGCCCCTCCCGCTCCTGCTCCTGCTCCTGTTCCACCGTG
GGTCCCTTTGCAGCCAATGCAACTTGGACGTTTTTGGGGTCTCCG
GACACCATCTCTATGTCTTGGCCCTGATCCTGAGCCGCCCGGGGC
TCCTGGTCTTCCGCCTCCTCGTCCTCGTCCTCTTCCCCGTCCTCG
TCCATGGTTATCACCCCCTCTTCTTTGAGGTCCACTGCCGCCGGA
GCCTTCTGGTCCAGATGTGTCTCCCTTCTCTCCTAGGCCATTTCC
AGGTCCTGTACCTGGCCCCTCGTCAGACATGATTCACACTAAAAG
AGATCAATAGACATCTTTATTAGACGACGCTCAGTGAATACAGGG
AGTGCAGACTCCTGCCCCCTCCAACAGCCCCCCCACCCTCATCCC
CTTCATGGTCGCTGTCAGACAGATCCAGGTCTGAAAATTCCCCAT
CCTCCGAACCATCCTCGTCCTCATCACCAATTACTCGCAGCCCGG
AAAACTCCCGCTGAACATCCTCAAGATTTGCGTCCTGAGCCTCAA
GCCAGGCCTCAAATTCCTCGTCCCCCTTTTTGCTGGACGGTAGGG
ATGGGGATTCTCGGGACCCCTCCTCTTCCTCTTCAAGGTCACCAG
ACAGAGATGCTACTGGGGCAACGGAAGAAAAGCTGGGTGCGGCCT
GTGAGGATCAGCTTATCGATGATAAGCTGTCAAACATGAGAATTC
TTGAAGACGAAAGGGCCTCGTGATACGCCTATTTTTATAGGTTAA
TGTCATGATAATAATGGTTTCTTAGACGTCAGGTGGCACTTTTCG
GGGAAATGTGCGCGGAACCCCTATTTGTTTATTTTTCTAAATACA
TTCAAATATGTATCCGCTCATGAGACAATAACCCTGATAAATGCT
TCAATAATATTGAAAAAGGAAGAGTATGAGTATTCAACATTTCCG
TGTCGCCCTTATTCCCTTTTTTGCGGCATTTTGCCTTCCTGTTTT
TGCTCACCCAGAAACGCTGGTGAAAGTAAAAGATGCTGAAGATCA
GTTGGGTGCACGAGTGGGTTACATCGAACTGGATCTCAACAGCGG
TAAGATCCTTGAGAGTTTTCGCCCCGAAGAACGTTTTCCAATGAT
GAGCACTTTTAAAGTTCTGCTATGTGGCGCGGTATTATCCCGTGT
TGACGCCGGGCAAGAGCAACTCGGTCGCCGCATACACTATTCTCA
GAATGACTTGGTTGAGTACTCACCAGTCACAGAAAAGCATCTTAC
GGATGGCATGACAGTAAGAGAATTATGCAGTGCTGCCATAACCAT
GAGTGATAACACTGCGGCCAACTTACTTCTGACAACGATCGGAGG
ACCGAAGGAGCTAACCGCTTTTTTGCACAACATGGGGGATCATGT
AACTCGCCTTGATCGTTGGGAACCGGAGCTGAATGAAGCCATACC
AAACGACGAGCGTGACACCACGATGCCTGCAGCAATGGCAACAAC
GTTGCGCAAACTATTAACTGGCGAACTACTTACTCTAGCTTCCCG
GCAACAATTAATAGACTGGATGGAGGCGGATAAAGTTGCAGGACC
ACTTCTGCGCTCGGCCCTTCCGGCTGGCTGGTTTATTGCTGATAA
ATCTGGAGCCGGTGAGCGTGGGTCTCGCGGTATCATTGCAGCACT
GGGGCCAGATGGTAAGCCCTCCCGTATCGTAGTTATCTACACGAC
GGGGAGTCAGGCAACTATGGATGAACGAAATAGACAGATCGCTGA
GATAGGTGCCTCACTGATTAAGCATTGGTAACTGTCAGACCAAGT
TTACTCATATATACTTTAGATTGATTTAAAACTTCATTTTTAATT
TAAAAGGATCTAGGTGAAGATCCTTTTTGATAATCTCATGACCAA
AATCCCTTAACGTGAGTTTTCGTTCCACTGAGCGTCAGACCCCGT
AGAAAAGATCAAAGGATCTTCTTGAGATCCTTTTTTTCTGCGCGT
AATCTGCTGCTTGCAAACAAAAAAACCACCGCTACCAGCGGTGGT
TTGTTTGCCGGATCAAGAGCTACCAACTCTTTTTCCGAAGGTAAC
TGGCTTCAGCAGAGCGCAGATACCAAATACTGTCCTTCTAGTGTA
GCCGTAGTTAGGCCACCACTTCAAGAACTCTGTAGCACCGCCTAC
ATACCTCGCTCTGCTAATCCTGTTACCAGTGGCTGCTGCCAGTGG
CGATAAGTCGTGTCTTACCGGGTTGGACTCAAGACGATAGTTACC
GGATAAGGCGCAGCGGTCGGGCTGAACGGGGGGTTCGTGCACACA
GCCCAGCTTGGAGCGAACGACCTACACCGAACTGAGATACCTACA
GCGTGAGCTATGAGAAAGCGCCACGCTTCCCGAAGGGAGAAAGGC
GGACAGGTATCCGGTAAGCGGCAGGGTCGGAACAGGAGAGCGCAC
GAGGGAGCTTCCAGGGGGAAACGCCTGGTATCTTTATAGTCCTGT
CGGGTTTCGCCACCTCTGACTTGAGCGTCGATTTTTGTGATGCTC
GTCAGGGGGGCGGAGCCTATGGAAAAACGCCAGCAACGCGGCCTT
TTTACGGTTCCTGGCCTTTTGCTGGCCTTTTGCTCACATGTTCTT
TCCTGCGTTATCCCCTGATTCTGTGGATAACCGTATTACCGCCTT
TGAGTGAGCTGATACCGCTCGCCGCAGCCGAACGACCGAGCGCAG
CGAGTCAGTGAGCGAGGAAGCGGAAGAGCGCCTGATGCGGTATTT
TCTCCTTACGCATCTGTGCGGTATTTCACACCGCATATGGTGCAC
TCTCAGTACAATCTGCTCTGATGCCGCATAGTTAAGCCAGCTGTG
GAATGTGTGTCAGTTAGGGTGTGGAAAGTCCCCAGGCTCCCCAGC
AGGCAGAAGTATGCAAAGCATGCATCTCAATTAGTCAGCAACCAG
GTGTGGAAAGTCCCCAGGCTCCCCAGCAGGCAGAAGTATGCAAAG
CATGCATCTCAATTAGTCAGCAACCATAGTCCCGCCCCTAACTCC
GCCCATCCCGCCCCTAACTCCGCCCAGTTCCGCCCATTCTCCGCC
CCATGGCTGACTAATTTTTTTTATTTATGCAGAGGCCGAGGCCGC
CTCGGCCTCTGAGCTATTCCAGAAGTAGTGAGGAGGCTTTTTTGG
AGGCCTAGGCTTTTGCAAAAAGCTTGCATGCCTGCAGGTCGGCCG
CCACGACCGGTGCCGCCACCATCCCCTGACCCACGCCCCTGACCC
CTCACAAGGAGACGACCTTCCATGACCGAGTACAAGCCCACGGTG
CGCCTCGCCACCCGCGACGACGTCCCCCGGGCCGTACGCACCCTC
GCCGCCGCGTTCGCCGACTACCCCGCCACGCGCCACACCGTCGAC
CCGGACCGCCACATCGAGCGGGTCACCGAGCTGCAAGAACTCTTC
CTCACGCGCGTCGGGCTCGACATCGGCAAGGTGTGGGTCGCGGAC
GACGGCGCCGCGGTGGCGGTCTGGACCACGCCGGAGAGCGTCGAA
GCGGGGGCGGTGTTCGCCGAGATCGGCCCGCGCATGGCCGAGTTG
AGCGGTTCCCGGCTGGCCGCGCAGCAACAGATGGAAGGCCTCCTG
GCGCCGCACCGGCCCAAGGAGCCCGCGTGGTTCCTGGCCACCGTC
GGCGTCTCGCCCGACCACCAGGGCAAGGGTCTGGGCAGCGCCGTC
GTGCTCCCCGGAGTGGAGGCGGCCGAGCGCGCCGGGGTGCCCGCC
TTCCTGGAGACCTCCGCGCCCCGCAACCTCCCCTTCTACGAGCGG
CTCGGCTTCACCGTCACCGCCGACGTCGAGGTGCCCGAAGGACCG
CGCACCTGGTGCATGACCCGCAAGCCCGGTGCCTGACGCCCGCCC
CACGACCCGCAGCGCCCGACCGAAAGGAGCGCACGACCCCATGGC
TCCGACCGAAGCCGACCCGGGCGGCCCCGCCGACCCCGCACCCGC
CCCCGAGGCCCACCGACTCTAGAGGATCATAATCAGCCATACCAC
ATTTGTAGAGGTTTTACTTGCTTTAAAAAACCTCCCACACCTCCC
CCTGAACCTGAAACATAAAATGAATGCAATTGTTGTTGTTAACTT
GTTTATTGCAGCTTATAATGGTTACAAATAAAGCAATAGCATCAC
AAATTTCACAAATAAAGCATTTTTTTCACTGCATTCTAGTTGTGG
TTTGTCCAAACTCATCAATGTATCTTATCATGTCTGGATCACTCG
CCGATAGTGGAAACCGACGCCCCAGCACTCGTCCGAGGGCAAAGG
AATAGGGGAGATGGGGGAGGCTAACTGAAACACGGAAGGAGACAA
TACCGGAAGGAACCCGCGCTATGACGGCAATAAAAAGACAGAATA
AAACGCACGGGTGTTGGGTCGTTTGTTCATAAACGCGGGGTTCGG
TCCCAGGGCTGGCACTCTGTCGATACCCCACCGAGACCCCATTGG
GGCCAATACGCCCGCGTTTCTTCCTTTTCCCCACCCCACCCCCCA
AGTTCGGGTGAAGGCCCAGGGCTCGCAGCCAACGTCGGGGCGGCA
GGCCCTGCCATAGCCACTGGCCCCGTGGGTTAGGGACGGGGTCCC
CCATGGGGAATGGTTTATGGTTCGTGGGGGTTATTATTTTGGGCG
TTGCGTGGGGTCTGGTCCACGACTGGACTGAGCAGACAGACCCAT
GGTTTTTGGATGGCCTGGGCATGGACCGCATGTACTGGCGCGACA
CGAACACCGGGCGTCTGTGGCTGCCAAACACCCCCGACCCCCAAA
AACCACCGCGCGGATTTCTGGCGTGCCAAGCTAGTCGACCAATTC
TCATGTTTGACAGCTTATCATCGCAGATCCGGGCAACGTTGTTGC
ATTGCTGCAGGCGCAGAACTGGTAGGTATGGAAGATCTCTAGAAG
CTGGGTACCAGCTGCTAGCAAGCTTGCTAGCGGCCGGCTCGAGTT
TACTCCCTATCAGTGATAGAGAACGTATGTCGAGTTTACTCCCTA
TCAGTGATAGAGAACGATGTCGAGTTTACTCCCTATCAGTGATAG
AGAACGTATGTCGAGTTTACTCCCTATCAGTGATAGAGAACGTAT
GTCGAGTTTACTCCCTATCAGTGATAGAGAACGTATGTCGAGTTT
ATCCCTATCAGTGATAGAGAACGTATGTCGAGTTTACTCCCTATC
AGTGATAGAGAACGTATGTCGAGGTAGGCGTGTACGGTGGGAGGC
CTATATAAGCAGAGCTCGTT (SEQ ID NO: 41)
LINE-1 ORF2-NLS mRNA (SEQ ID NO: 42)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGTGAGACAGCCGTCAGACCATCAAGACTAGGA
AGAAACTGCATCAACTAATGAGCAAAATCACCAGCTAACATCATA
GTATACATGACCGGCTCTAACTCACATATCACCATCCTTACACTT
AACATTAACGGCCTCAACTCAGCTATCAAGCGCCATCGGCTGGCC
AGCTGGATCAAATCACAGGATCCAAGCGTTTGTTGCATCCAAGAG
ACCCACCTGACCTGTAGAGATACTCACCGCCTCAAGATCAAGGGA
TGGCGAAAGATTTATCAGGCGAACGGTAAGCAGAAGAAAGCCGGA
GTCGCAATTCTGGTCTCAGACAAGACGGATTTCAAGCCCACCAAA
ATTAAGCGTGATAAGGAAGGTCACTATATTATGGTGAAAGGCAGC
ATACAGCAGGAAGAACTTACCATATTGAACATCTACGCGCCAAAC
ACCGGCGCACCTCGCTTTATCAAACAGGTCCTGTCCGATCTGCAG
CGAGATCTGGATTCTCATACGTTGATTATGGGTGATTTCAATACA
CCATTGAGCACCCTGGATCGCAGCACCAGGCAAAAGGTAAATAAA
GACACGCAAGAGCTCAATAGCGCACTGCATCAGGCAGATCTCATT
GATATTTATCGCACTCTTCATCCTAAGAGTACCGAGTACACATTC
TTCAGCGCCCCACATCATACATACTCAAAGATCGATCATATCGTC
GGCTCAAAGGCTCTGCTGTCAAAGTGCAAGCGCACAGAGATAATT
ACAAATTACCTGTCAGATCATAGCGCGATCAAGCTCGAGCTGAGA
ATCAAGAACCTGACCCAGAGCCGGAGTACCACTTGGAAGCTTAAT
AACCTGCTGCTCAACGATTATTGGGTCCACAATGAGATGAAGGCA
GAGATTAAAATGTTCTTCGAAACAAATGAGAATAAGGATACTACC
TATCAAAACCTTTGGGATGCCTTTAAGGCCGTCTGCAGAGGCAAG
TTCATCGCCCTCAACGCCTATAAAAGAAAACAAGAGAGATCTAAG
ATCGATACTCTCACCTCTCAGCTGAAGGAGTTGGAGAAACAGGAA
CAGACCCACTCCAAGGCGTCAAGACGGCAGGAGATCACAAAGATT
CGCGCCGAGTTGAAAGAGATCGAAACCCAAAAGACTCTTCAGAAA
ATTAACGAGTCTCGTAGTTGGTTCTTCGAGCGGATTAATAAGATA
GACAGACCTCTGGCACGACTGATTAAGAAGAAGCGCGAAAAGAAC
CAGATTGATACCATCAAGAACGACAAGGGCGACATCACTACTGAC
CCGACCGAGATCCAGACCACTATTCGGGAGTATTATAAGCATTTG
TATGCTAACAAGCTTGAGAACCTGGAAGAGATGGACACTTTTCTG
GATACCTATACTCTGCCACGGCTTAATCAAGAGGAAGTCGAGTCC
CTCAACCGCCCAATTACAGGAAGCGAGATTGTGGCCATAATTAAC
TCCCTGCCGACAAAGAAATCTCCTGGTCCGGACGGGTTTACAGCT
GAGTTTTATCAACGGTATATGGAAGAGCTTGTACCGTTTCTGCTC
AAGCTCTTTCAGTCTATAGAAAAGGAAGGCATCTTGCCCAATTCC
TTCTACGAAGCTTCTATAATACTTATTCCCAAACCAGGACGCGAT
ACCACAAAGAAGGAAAACTTCCGGCCCATTAGTCTCATGAATATC
GACGCTAAAATATTGAACAAGATTCTCGCCAACAGAATCCAACAA
CATATTAAGAAATTGATACATCACGACCAGGTGGGGTTTATACCT
GGCATGCAGGGCTGGTTTAACATCCGGAAGAGTATTAACGTCATT
CAACACATTAATAGAGCTAAGGATAAGAATCATATGATCATCTCT
ATAGACGCGGAAAAGGCATTCGATAAGATTCAGCAGCCATTTATG
CTCAAGACTCTGAACAAACTCGGCATCGACGGAACATATTTTAAG
ATTATTCGCGCAATTTACGATAAGCCGACTGCTAACATTATCCTT
AACGGCCAAAAGCTCGAGGCCTTTCCGCTCAAGACTGGAACCCGC
CAAGGCTGTCCCCTCTCCCCGCTTTTGTTTAATATTGTACTCGAG
GTGCTGGCTAGGGCTATTCGTCAAGAGAAAGAGATTAAAGGGATA
CAGCTCGGGAAGGAAGAGGTCAAGCTTTCCTTGTTCGCCGATGAT
ATGATTGTGTACCTGGAGAATCCTATTGTGTCTGCTCAGAACCTT
CTTAAACTTATTTCTAACTTTAGCAAGGTCAGCGGCTATAAGATT
AACGTCCAGAAATCTCAGGCCTTTCTGTACACAAATAATCGACAG
ACCGAATCCCAGATAATGGGTGAGCTTCCGTTTGTCATAGCCAGC
AAAAGGATAAAGTATCTCGGAATCCAGCTGACACGAGACGTTAAA
GATTTGTTTAAGGAAAATTACAAGCCTCTCCTGAAAGAGATTAAG
GAAGATACTAATAAGTGGAAGAATATCCCCTGTTCATGGGTTGGC
AGAATCAACATAGTGAAGATGGCAATACTTCCTAAAGTGATATAT
CGCTTTAACGCCATCCCAATTAAACTGCCTATGACCTTCTTTACG
GAGCTCGAGAAAACAACCCTTAAATTTATATGGAATCAAAAGAGA
GCAAGAATAGCGAAGTCCATCTTGAGCCAGAAGAATAAGGCCGGT
GGGATTACTTTGCCTGATTTTAAGTTGTATTATAAAGCCACAGTA
ACTAAGACAGCCTGGTATTGGTATCAGAATAGAGACATCGACCAG
TGGAATCGGACCGAACCATCAGAGATAATGCCCCACATCTATAAT
TACCTTATATTCGATAAGCCAGAAAAGAATAAACAGTGGGGCAAA
GACAGCCTCTTCAACAAGTGGTGTTGGGAGAATTGGCTGGCCATA
TGCCGGAAACTCAAGCTCGACCCCTTTCTTACACCCTACACTAAA
ATCAACAGTAGGTGGATCAAGGACTTGAATGTCAAGCCAAAGACT
ATAAAGACACTGGAAGAGAATCTTGGGATCACAATACAAGATATA
GGCGTCGGCAAAGATTTTATGTCAAAGACGCCCAAGGCCATGGCC
ACTAAGGATAAGATTGATAAGTGGGACCTTATTAAGCTCAAAAGC
TTCTGTACTGCCAAGGAGACCACGATCAGAGTTAATAGGCAGCCC
ACTACATGGGAAAAGATTTTCGCCACTTATTCATCAGATAAGGGG
TTGATAAGCAGAATATATAACGAGCTGAAGCAGATCTACAAGAAG
AAAACGAATAATCCCATCAAGAAGTGGGCAAAAGATATGAACAGG
CATTTTAGCAAAGAGGATATCTACGCCGCGAAGAAGCATATGAAG
AAGTGTAGTTCAAGCTTGGCCATTCGTGAGATGCAGATTAAGACG
ACCATGCGATACCACCTTACCCCAGTGAGGATGGCAATTATCAAG
AAATCTGGCAATAATAGATGTTGGCGGGGCTGTGGCGAGATTGGC
ACCCTGCTCCATTGCTGGTGGGATTGCAAGCTGGTGCAGCCGCTT
TGGAAATCAGTCTGGCGCTTTCTGAGGGACCTCGAGCTTGAGATT
CCCTTCGATCCCGCAATTCCCTTGCTCGGAATCTATCCTAACGAA
TACAAGAGCTGTTGTTACAAGGATACGTGTACCCGGATGTTCATC
GCGGCCTTGTTTACGATAGCTAAGACGTGGAATCAGCCTAAGTGC
CCCACAATGATCGATTGGATCAAGAAAATGTGGCATATTTATACC
ATGGAGTATTACGCAGCAATTAAGAATGACGAATTTATTTCCTTC
GTTGGGACCTGGATGAAGCTGGAGACTATTATTCTGAGCAAGCTG
TCTCAGGAGCAAAAGACAAAGCATAGAATCTTCTCTCTCATTGGT
GGTAACGACTACAAAGACGATGACGACAAGCCCGCCGCCAAGAGG
GTGAAGCTGGACTAAAGCGCTTCTAGAAGTTGTCTCCTCCTGCAC
TGACTGACTGATACAATCGATTTCTGGATCCGCAGGCCTAATCAA
CCTCTGGATTACAAAATTTGTGAAAGATTGACTGGTATTCTTAAC
TATGTTGCTCCTTTTACGCTATGTGGATACGCTGCTTTAATGCCT
TTGTATCATGCTATTGCTTCCCGTATGGCTTTCATTTTCTCCTCC
TTGTATAAATCCTGGTTGCTGTCTCTTTATGAGGAGTTGTGGCCC
GTTGTCAGGCAACGTGGCGTGGTGTGCACTGTGTTTGCTGACGCA
ACCCCCACTGGTTGGGGCATTGCCACCACCTGTCAGCTCCTTTCC
GGGACTTTCGCTTTCCCCCTCCCTATTGCCACGGCGGAACTCATC
GCCGCCTGCCTTGCCCGCTGCTGGACAGGGGCTCGGCTGTTGGGC
ACTGACAATTCCGTGGTGTTGTCGGGGAAGCTGACGTCCTTTCCA
TGGCTGCTCGCCTGTGTTGCCACCTGGATTCTGCGCGGGACGTCC
TTCTGCTACGTCCCTTCGGCCCTCAATCCAGCGGACCTTCCTTCC
CGCTGAGAGACACAAAAAATTCCAACACACTATTGCAATGAAAAT
AAATTTCCTTTATTAGCCAGAAGTCAGATGCTCAAGGGGCTTCAT
GATGTCCCCATAATTTTTGGCAGAGGGAAAAAGATCTCAGTGGTA
TTTGTGAGCCAGGGCATTGGCCTTCTGATAGGCAGCCTGCACCTG
AGGAGTGCGGCCGCTTTACTTGTACAGCTCGTCCATGCCGAGAGT
GATCCCGGCGGCGGTCACGAACTCCAGCAGGACCATGTGATCGCG
CTTCTCGTTGGGGTCTTTGCTCAGGGCGGACTGGGTGCTCAGGTA
GTGGTTGTCGGGCAGCAGCACGGGGCCGTCGCCGATGGGGGTGTT
CTGCTGGTAGTGGTCGGCGAGCTGCACGCTGCCGTCCTCGATGTT
GTGGCGGATCTTGAAGTTCACCTTGATGCCGTTCTTCTGCTTGTC
GGCCATGATATAGACGTTGTGGCTGTTGTAGTTGTACTCCAGCTT
GTGCCCCAGGATGTTGCCGTCCTCCTTGAAGTCGATGCCCTTCAG
CTCGATGCGGTTCACCAGGGTGTCGCCCTCGAACTTCACCTCGGC
GCGGGTCTTGTAGTTGCCGTCGTCCTTGAAGAAGATGGTGCGCTC
CTGGACGTAGCCTTCGGGCATGGCGGACTTGAAGAAGTCGTGCTG
CTTCATGTGGTCGGGGTAGCGGCTGAAGCACTGCACGCCGTAGGT
CAGGGTGGTCACGAGGGTGGGCCAGGGCACGGGCAGCTTGCCGGT
GGTGCAGATGAACTTCAGGGTCAGCTTGCCGTAGGTGGCATCGCC
CTCGCCCTCGCCGGACACGCTGAACTTGTGGCCGTTTACGTCGCC
GTCCAGCTCGACCAGGATGGGCACCACCCCGGTGAACAGCTCCTC
GCCCTTGCTCACCATGGTGGCGGGATCTGACGGTTCACTAAACCA
GCTCTGCTTATATAGACCTCCCACCGTACACGCCTACCGCCCATT
TGCGTCAATGGGGCGGAGTTGTTACGACATTTTGGAAAGTCCCGT
TGATTTTGGTGCCAAAACAAACTCCCATTGACGTCAATGGGGTGG
AGACTTGGAAATCCCCGTGAGTCAAACCGCTATCCACGCCCATTG
ATGTACTGCCAAAACCGCATCACCATGGTAATAGCGATGACTAAT
ACGTAGATGTACTGCCAAGTAGGAAAGTCCCATAAGGTCATGTAC
TGGGCATAATGCCAGGCGGGCCATTTACCGTCATTGACGTCAATA
GGGGGCGTACTTGGCATATGATACACTTGATGTACTGCCAAGTGG
GCAGTTTACCGTAAATACTCCACCCATTGACGTCAATGGAAAGTC
CCTATTGGCGTTACTATGGGAACATACGTCATTATTGACGTCAAT
GGGCGGGGGTCGTTGGGCGGTCAGCCAGGCGGGCCATTTACCGTA
AGTTATGTAACGGGCCTGCTGCCGGCTCTGCGGCCTCTTCCGCGT
CTTCGCCTTCGCCCTCAGACGAGTCGGATCTCCCTTTGGGCCGCC
TCCCCGCCTGTCTAGCTTGACTGACTGAGATACAGCGTACCTTCA
GCTCACAGACATGATAAGATACATTGATGAGTTTGGACAAACCAC
AACTAGAATGCAGTGAAAAAAATGCTTTATTTGTGAAATTTGTGA
TGCTATTGCTTTATTTGTAACCATTATAAGCTGCAATAAACAAGT
T (SEQ ID NO: 42)
LINE-1 alu mRNA GFP (SEQ ID NO: 43)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGTGAGACAGCCGTCAGACCATCAAGACTAGGA
AGAAACTGCATCAACTAATGAGCAAAATCACCAGCTAACATCATA
GTATACATGACCGGCTCTAACTCACATATCACCATCCTTACACTT
AACATTAACGGCCTCAACTCAGCTATCAAGCGCCATCGGCTGGCC
AGCTGGATCAAATCACAGGATCCAAGCGTTTGTTGCATCCAAGAG
ACCCACCTGACCTGTAGAGATACTCACCGCCTCAAGATCAAGGGA
TGGCGAAAGATTTATCAGGCGAACGGTAAGCAGAAGAAAGCCGGA
GTCGCAATTCTGGTCTCAGACAAGACGGATTTCAAGCCCACCAAA
ATTAAGCGTGATAAGGAAGGTCACTATATTATGGTGAAAGGCAGC
ATACAGCAGGAAGAACTTACCATATTGAACATCTACGCGCCAAAC
ACCGGCGCACCTCGCTTTATCAAACAGGTCCTGTCCGATCTGCAG
CGAGATCTGGATTCTCATACGTTGATTATGGGTGATTTCAATACA
CCATTGAGCACCCTGGATCGCAGCACCAGGCAAAAGGTAAATAAA
GACACGCAAGAGCTCAATAGCGCACTGCATCAGGCAGATCTCATT
GATATTTATCGCACTCTTCATCCTAAGAGTACCGAGTACACATTC
TTCAGCGCCCCACATCATACATACTCAAAGATCGATCATATCGTC
GGCTCAAAGGCTCTGCTGTCAAAGTGCAAGCGCACAGAGATAATT
ACAAATTACCTGTCAGATCATAGCGCGATCAAGCTCGAGCTGAGA
ATCAAGAACCTGACCCAGAGCCGGAGTACCACTTGGAAGCTTAAT
AACCTGCTGCTCAACGATTATTGGGTCCACAATGAGATGAAGGCA
GAGATTAAAATGTTCTTCGAAACAAATGAGAATAAGGATACTACC
TATCAAAACCTTTGGGATGCCTTTAAGGCCGTCTGCAGAGGCAAG
TTCATCGCCCTCAACGCCTATAAAAGAAAACAAGAGAGATCTAAG
ATCGATACTCTCACCTCTCAGCTGAAGGAGTTGGAGAAACAGGAA
CAGACCCACTCCAAGGCGTCAAGACGGCAGGAGATCACAAAGATT
CGCGCCGAGTTGAAAGAGATCGAAACCCAAAAGACTCTTCAGAAA
ATTAACGAGTCTCGTAGTTGGTTCTTCGAGCGGATTAATAAGATA
GACAGACCTCTGGCACGACTGATTAAGAAGAAGCGCGAAAAGAAC
CAGATTGATACCATCAAGAACGACAAGGGCGACATCACTACTGAC
CCGACCGAGATCCAGACCACTATTCGGGAGTATTATAAGCATTTG
TATGCTAACAAGCTTGAGAACCTGGAAGAGATGGACACTTTTCTG
GATACCTATACTCTGCCACGGCTTAATCAAGAGGAAGTCGAGTCC
CTCAACCGCCCAATTACAGGAAGCGAGATTGTGGCCATAATTAAC
TCCCTGCCGACAAAGAAATCTCCTGGTCCGGACGGGTTTACAGCT
GAGTTTTATCAACGGTATATGGAAGAGCTTGTACCGTTTCTGCTC
AAGCTCTTTCAGTCTATAGAAAAGGAAGGCATCTTGCCCAATTCC
TTCTACGAAGCTTCTATAATACTTATTCCCAAACCAGGACGCGAT
ACCACAAAGAAGGAAAACTTCCGGCCCATTAGTCTCATGAATATC
GACGCTAAAATATTGAACAAGATTCTCGCCAACAGAATCCAACAA
CATATTAAGAAATTGATACATCACGACCAGGTGGGGTTTATACCT
GGCATGCAGGGCTGGTTTAACATCCGGAAGAGTATTAACGTCATT
CAACACATTAATAGAGCTAAGGATAAGAATCATATGATCATCTCT
ATAGACGCGGAAAAGGCATTCGATAAGATTCAGCAGCCATTTATG
CTCAAGACTCTGAACAAACTCGGCATCGACGGAACATATTTTAAG
ATTATTCGCGCAATTTACGATAAGCCGACTGCTAACATTATCCTT
AACGGCCAAAAGCTCGAGGCCTTTCCGCTCAAGACTGGAACCCGC
CAAGGCTGTCCCCTCTCCCCGCTTTTGTTTAATATTGTACTCGAG
GTGCTGGCTAGGGCTATTCGTCAAGAGAAAGAGATTAAAGGGATA
CAGCTCGGGAAGGAAGAGGTCAAGCTTTCCTTGTTCGCCGATGAT
ATGATTGTGTACCTGGAGAATCCTATTGTGTCTGCTCAGAACCTT
CTTAAACTTATTTCTAACTTTAGCAAGGTCAGCGGCTATAAGATT
AACGTCCAGAAATCTCAGGCCTTTCTGTACACAAATAATCGACAG
ACCGAATCCCAGATAATGGGTGAGCTTCCGTTTGTCATAGCCAGC
AAAAGGATAAAGTATCTCGGAATCCAGCTGACACGAGACGTTAAA
GATTTGTTTAAGGAAAATTACAAGCCTCTCCTGAAAGAGATTAAG
GAAGATACTAATAAGTGGAAGAATATCCCCTGTTCATGGGTTGGC
AGAATCAACATAGTGAAGATGGCAATACTTCCTAAAGTGATATAT
CGCTTTAACGCCATCCCAATTAAACTGCCTATGACCTTCTTTACG
GAGCTCGAGAAAACAACCCTTAAATTTATATGGAATCAAAAGAGA
GCAAGAATAGCGAAGTCCATCTTGAGCCAGAAGAATAAGGCCGGT
GGGATTACTTTGCCTGATTTTAAGTTGTATTATAAAGCCACAGTA
ACTAAGACAGCCTGGTATTGGTATCAGAATAGAGACATCGACCAG
TGGAATCGGACCGAACCATCAGAGATAATGCCCCACATCTATAAT
TACCTTATATTCGATAAGCCAGAAAAGAATAAACAGTGGGGCAAA
GACAGCCTCTTCAACAAGTGGTGTTGGGAGAATTGGCTGGCCATA
TGCCGGAAACTCAAGCTCGACCCCTTTCTTACACCCTACACTAAA
ATCAACAGTAGGTGGATCAAGGACTTGAATGTCAAGCCAAAGACT
ATAAAGACACTGGAAGAGAATCTTGGGATCACAATACAAGATATA
GGCGTCGGCAAAGATTTTATGTCAAAGACGCCCAAGGCCATGGCC
ACTAAGGATAAGATTGATAAGTGGGACCTTATTAAGCTCAAAAGC
TTCTGTACTGCCAAGGAGACCACGATCAGAGTTAATAGGCAGCCC
ACTACATGGGAAAAGATTTTCGCCACTTATTCATCAGATAAGGGG
TTGATAAGCAGAATATATAACGAGCTGAAGCAGATCTACAAGAAG
AAAACGAATAATCCCATCAAGAAGTGGGCAAAAGATATGAACAGG
CATTTTAGCAAAGAGGATATCTACGCCGCGAAGAAGCATATGAAG
AAGTGTAGTTCAAGCTTGGCCATTCGTGAGATGCAGATTAAGACG
ACCATGCGATACCACCTTACCCCAGTGAGGATGGCAATTATCAAG
AAATCTGGCAATAATAGATGTTGGCGGGGCTGTGGCGAGATTGGC
ACCCTGCTCCATTGCTGGTGGGATTGCAAGCTGGTGCAGCCGCTT
TGGAAATCAGTCTGGCGCTTTCTGAGGGACCTCGAGCTTGAGATT
CCCTTCGATCCCGCAATTCCCTTGCTCGGAATCTATCCTAACGAA
TACAAGAGCTGTTGTTACAAGGATACGTGTACCCGGATGTTCATC
GCGGCCTTGTTTACGATAGCTAAGACGTGGAATCAGCCTAAGTGC
CCCACAATGATCGATTGGATCAAGAAAATGTGGCATATTTATACC
ATGGAGTATTACGCAGCAATTAAGAATGACGAATTTATTTCCTTC
GTTGGGACCTGGATGAAGCTGGAGACTATTATTCTGAGCAAGCTG
TCTCAGGAGCAAAAGACAAAGCATAGAATCTTCTCTCTCATTGGT
GGTAACGACTACAAAGACGATGACGACAAGTAAAGCGGCCGGGCG
CGGTGGCTCACGCCTGTAATCCCAGCACTTTGGGAGGCCGAGGCG
GGAGGATCGCAGTTCGAGACCAGCGCGAGACCCCGTCTCTACAAA
AATACAAAAATTAGCTTCTAGAAGTTGTCTCCTCCTGCACTGACT
GACTGATACAATCGATTTCTGGATCCGCAGGCCTAATCAACCTCT
GGATTACAAAATTTGTGAAAGATTGACTGGTATTCTTAACTATGT
TGCTCCTTTTACGCTATGTGGATACGCTGCTTTAATGCCTTTGTA
TCATGCTATTGCTTCCCGTATGGCTTTCATTTTCTCCTCCTTGTA
TAAATCCTGGTTGCTGTCTCTTTATGAGGAGTTGTGGCCCGTTGT
CAGGCAACGTGGCGTGGTGTGCACTGTGTTTGCTGACGCAACCCC
CACTGGTTGGGGCATTGCCACCACCTGTCAGCTCCTTTCCGGGAC
TTTCGCTTTCCCCCTCCCTATTGCCACGGCGGAACTCATCGCCGC
CTGCCTTGCCCGCTGCTGGACAGGGGCTCGGCTGTTGGGCACTGA
CAATTCCGTGGTGTTGTCGGGGAAGCTGACGTCCTTTCCATGGCT
GCTCGCCTGTGTTGCCACCTGGATTCTGCGCGGGACGTCCTTCTG
CTACGTCCCTTCGGCCCTCAATCCAGCGGACCTTCCTTCCCGCTG
AGAGACACAAAAAATTCCAACACACTATTGCAATGAAAATAAATT
TCCTTTATTAGCCAGAAGTCAGATGCTCAAGGGGCTTCATGATGT
CCCCATAATTTTTGGCAGAGGGAAAAAGATCTCAGTGGTATTTGT
GAGCCAGGGCATTGGCCTTCTGATAGGCAGCCTGCACCTGAGGAG
TGCGGCCGCTTTACTTGTACAGCTCGTCCATGCCGAGAGTGATCC
CGGCGGCGGTCACGAACTCCAGCAGGACCATGTGATCGCGCTTCT
CGTTGGGGTCTTTGCTCAGGGCGGACTGGGTGCTCAGGTAGTGGT
TGTCGGGCAGCAGCACGGGGCCGTCGCCGATGGGGGTGTTCTGCT
GGTAGTGGTCGGCGAGCTGCACGCTGCCGTCCTCGATGTTGTGGC
GGATCTTGAAGTTCACCTTGATGCCGTTCTTCTGCTTGTCGGCCA
TGATATAGACGTTGTGGCTGTTGTAGTTGTACTCCAGCTTGTGCC
CCAGGATGTTGCCGTCCTCCTTGAAGTCGATGCCCTTCAGCTCGA
TGCGGTTCACCAGGGTGTCGCCCTCGAACTTCACCTCGGCGCGGG
TCTTGTAGTTGCCGTCGTCCTTGAAGAAGATGGTGCGCTCCTGGA
CGTAGCCTTCGGGCATGGCGGACTTGAAGAAGTCGTGCTGCTTCA
TGTGGTCGGGGTAGCGGCTGAAGCACTGCACGCCGTAGGTCAGGG
TGGTCACGAGGGTGGGCCAGGGCACGGGCAGCTTGCCGGTGGTGC
AGATGAACTTCAGGGTCAGCTTGCCGTAGGTGGCATCGCCCTCGC
CCTCGCCGGACACGCTGAACTTGTGGCCGTTTACGTCGCCGTCCA
GCTCGACCAGGATGGGCACCACCCCGGTGAACAGCTCCTCGCCCT
TGCTCACCATGGTGGCGGGATCTGACGGTTCACTAAACCAGCTCT
GCTTATATAGACCTCCCACCGTACACGCCTACCGCCCATTTGCGT
CAATGGGGCGGAGTTGTTACGACATTTTGGAAAGTCCCGTTGATT
TTGGTGCCAAAACAAACTCCCATTGACGTCAATGGGGTGGAGACT
TGGAAATCCCCGTGAGTCAAACCGCTATCCACGCCCATTGATGTA
CTGCCAAAACCGCATCACCATGGTAATAGCGATGACTAATACGTA
GATGTACTGCCAAGTAGGAAAGTCCCATAAGGTCATGTACTGGGC
ATAATGCCAGGCGGGCCATTTACCGTCATTGACGTCAATAGGGGG
CGTACTTGGCATATGATACACTTGATGTACTGCCAAGTGGGCAGT
TTACCGTAAATACTCCACCCATTGACGTCAATGGAAAGTCCCTAT
TGGCGTTACTATGGGAACATACGTCATTATTGACGTCAATGGGCG
GGGGTCGTTGGGCGGTCAGCCAGGCGGGCCATTTACCGTAAGTTA
TGTAACGGGCCTGCTGCCGGCTCTGCGGCCTCTTCCGCGTCTTCG
CCTTCGCCCTCAGACGAGTCGGATCTCCCTTTGGGCCGCCTCCCC
GCCTGTCTAGCTTGACTGACTGAGATACAGCGTACCTTCAGCTCA
CAGACATGATAAGATACATTGATGAGTTTGGACAAACCACAACTA
GAATGCAGTGAAAAAAATGCTTTATTTGTGAAATTTGTGATGCTA
TTGCTTTATTTGTAACCATTATAAGCTGCAATAAACAAGTT
(SEQ ID NO: 43)
LINE-1 plasmid CVBE IRES GFP (SEQ ID NO: 44)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGTGAGACAGTTAAAACAGCCTGTGGGTTGATC
CCACCCACAGGCCCATTGGGCGCTAGCACTCTGGTATCACGGTAC
CTTTGTGCGCCTGTTTTATACCCCCTCCCCCAACTGTAACTTAGA
AGTAACACACACCGATCAACAGTCAGCGTGGCACACCAGCCACGT
TTTGATCAAGCACTTCTGTTACCCCGGACTGAGTATCAATAGACT
GCTCACGCGGTTGAAGGAGAAAGCGTTCGTTATCCGGCCAACTAC
TTCGAAAAACCTAGTAACACCGTGGAAGTTGCAGAGTGTTTCGCT
CAGCACTACCCCAGTGTAGATCAGGTCGATGAGTCACCGCATTCC
CCACGGGCGACCGTGGCGGTGGCTGCGTTGGCGGCCTGCCCATGG
GGAAACCCATGGGACGCTCTAATACAGACATGGTGCGAAGAGTCT
ATTGAGCTAGTTGGTAGTCCTCCGGCCCCTGAATGCGGCTAATCC
TAACTGCGGAGCACACACCCTCAAGCCAGAGGGCAGTGTGTCGTA
ACGGGCAACTCTGCAGCGGAACCGACTACTTTGGGTGTCCGTGTT
TCATTTTATTCCTATACTGGCTGCTTATGGTGACAATTGAGAGAT
CGTTACCATATAGCTATTGGATTGGCCATCCGGTGACTAATAGAG
CTATTATATATCCCTTTGTTGGGTTTATACCACTTAGCTTGAAAG
AGGTTAAAACATTACAATTCATTGTTAAGTTGAATACAGCAAATA
CATGACCGGCTCTAACTCACATATCACCATCCTTACACTTAACAT
TAACGGCCTCAACTCAGCTATCAAGCGCCATCGGCTGGCCAGCTG
GATCAAATCACAGGATCCAAGCGTTTGTTGCATCCAAGAGACCCA
CCTGACCTGTAGAGATACTCACCGCCTCAAGATCAAGGGATGGCG
AAAGATTTATCAGGCGAACGGTAAGCAGAAGAAAGCCGGAGTCGC
AATTCTGGTCTCAGACAAGACGGATTTCAAGCCCACCAAAATTAA
GCGTGATAAGGAAGGTCACTATATTATGGTGAAAGGCAGCATACA
GCAGGAAGAACTTACCATATTGAACATCTACGCGCCAAACACCGG
CGCACCTCGCTTTATCAAACAGGTCCTGTCCGATCTGCAGCGAGA
TCTGGATTCTCATACGTTGATTATGGGTGATTTCAATACACCATT
GAGCACCCTGGATCGCAGCACCAGGCAAAAGGTAAATAAAGACAC
GCAAGAGCTCAATAGCGCACTGCATCAGGCAGATCTCATTGATAT
TTATCGCACTCTTCATCCTAAGAGTACCGAGTACACATTCTTCAG
CGCCCCACATCATACATACTCAAAGATCGATCATATCGTCGGCTC
AAAGGCTCTGCTGTCAAAGTGCAAGCGCACAGAGATAATTACAAA
TTACCTGTCAGATCATAGCGCGATCAAGCTCGAGCTGAGAATCAA
GAACCTGACCCAGAGCCGGAGTACCACTTGGAAGCTTAATAACCT
GCTGCTCAACGATTATTGGGTCCACAATGAGATGAAGGCAGAGAT
TAAAATGTTCTTCGAAACAAATGAGAATAAGGATACTACCTATCA
AAACCTTTGGGATGCCTTTAAGGCCGTCTGCAGAGGCAAGTTCAT
CGCCCTCAACGCCTATAAAAGAAAACAAGAGAGATCTAAGATCGA
TACTCTCACCTCTCAGCTGAAGGAGTTGGAGAAACAGGAACAGAC
CCACTCCAAGGCGTCAAGACGGCAGGAGATCACAAAGATTCGCGC
CGAGTTGAAAGAGATCGAAACCCAAAAGACTCTTCAGAAAATTAA
CGAGTCTCGTAGTTGGTTCTTCGAGCGGATTAATAAGATAGACAG
ACCTCTGGCACGACTGATTAAGAAGAAGCGCGAAAAGAACCAGAT
TGATACCATCAAGAACGACAAGGGCGACATCACTACTGACCCGAC
CGAGATCCAGACCACTATTCGGGAGTATTATAAGCATTTGTATGC
TAACAAGCTTGAGAACCTGGAAGAGATGGACACTTTTCTGGATAC
CTATACTCTGCCACGGCTTAATCAAGAGGAAGTCGAGTCCCTCAA
CCGCCCAATTACAGGAAGCGAGATTGTGGCCATAATTAACTCCCT
GCCGACAAAGAAATCTCCTGGTCCGGACGGGTTTACAGCTGAGTT
TTATCAACGGTATATGGAAGAGCTTGTACCGTTTCTGCTCAAGCT
CTTTCAGTCTATAGAAAAGGAAGGCATCTTGCCCAATTCCTTCTA
CGAAGCTTCTATAATACTTATTCCCAAACCAGGACGCGATACCAC
AAAGAAGGAAAACTTCCGGCCCATTAGTCTCATGAATATCGACGC
TAAAATATTGAACAAGATTCTCGCCAACAGAATCCAACAACATAT
TAAGAAATTGATACATCACGACCAGGTGGGGTTTATACCTGGCAT
GCAGGGCTGGTTTAACATCCGGAAGAGTATTAACGTCATTCAACA
CATTAATAGAGCTAAGGATAAGAATCATATGATCATCTCTATAGA
CGCGGAAAAGGCATTCGATAAGATTCAGCAGCCATTTATGCTCAA
GACTCTGAACAAACTCGGCATCGACGGAACATATTTTAAGATTAT
TCGCGCAATTTACGATAAGCCGACTGCTAACATTATCCTTAACGG
CCAAAAGCTCGAGGCCTTTCCGCTCAAGACTGGAACCCGCCAAGG
CTGTCCCCTCTCCCCGCTTTTGTTTAATATTGTACTCGAGGTGCT
GGCTAGGGCTATTCGTCAAGAGAAAGAGATTAAAGGGATACAGCT
CGGGAAGGAAGAGGTCAAGCTTTCCTTGTTCGCCGATGATATGAT
TGTGTACCTGGAGAATCCTATTGTGTCTGCTCAGAACCTTCTTAA
ACTTATTTCTAACTTTAGCAAGGTCAGCGGCTATAAGATTAACGT
CCAGAAATCTCAGGCCTTTCTGTACACAAATAATCGACAGACCGA
ATCCCAGATAATGGGTGAGCTTCCGTTTGTCATAGCCAGCAAAAG
GATAAAGTATCTCGGAATCCAGCTGACACGAGACGTTAAAGATTT
GTTTAAGGAAAATTACAAGCCTCTCCTGAAAGAGATTAAGGAAGA
TACTAATAAGTGGAAGAATATCCCCTGTTCATGGGTTGGCAGAAT
CAACATAGTGAAGATGGCAATACTTCCTAAAGTGATATATCGCTT
TAACGCCATCCCAATTAAACTGCCTATGACCTTCTTTACGGAGCT
CGAGAAAACAACCCTTAAATTTATATGGAATCAAAAGAGAGCAAG
AATAGCGAAGTCCATCTTGAGCCAGAAGAATAAGGCCGGTGGGAT
TACTTTGCCTGATTTTAAGTTGTATTATAAAGCCACAGTAACTAA
GACAGCCTGGTATTGGTATCAGAATAGAGACATCGACCAGTGGAA
TCGGACCGAACCATCAGAGATAATGCCCCACATCTATAATTACCT
TATATTCGATAAGCCAGAAAAGAATAAACAGTGGGGCAAAGACAG
CCTCTTCAACAAGTGGTGTTGGGAGAATTGGCTGGCCATATGCCG
GAAACTCAAGCTCGACCCCTTTCTTACACCCTACACTAAAATCAA
CAGTAGGTGGATCAAGGACTTGAATGTCAAGCCAAAGACTATAAA
GACACTGGAAGAGAATCTTGGGATCACAATACAAGATATAGGCGT
CGGCAAAGATTTTATGTCAAAGACGCCCAAGGCCATGGCCACTAA
GGATAAGATTGATAAGTGGGACCTTATTAAGCTCAAAAGCTTCTG
TACTGCCAAGGAGACCACGATCAGAGTTAATAGGCAGCCCACTAC
ATGGGAAAAGATTTTCGCCACTTATTCATCAGATAAGGGGTTGAT
AAGCAGAATATATAACGAGCTGAAGCAGATCTACAAGAAGAAAAC
GAATAATCCCATCAAGAAGTGGGCAAAAGATATGAACAGGCATTT
TAGCAAAGAGGATATCTACGCCGCGAAGAAGCATATGAAGAAGTG
TAGTTCAAGCTTGGCCATTCGTGAGATGCAGATTAAGACGACCAT
GCGATACCACCTTACCCCAGTGAGGATGGCAATTATCAAGAAATC
TGGCAATAATAGATGTTGGCGGGGCTGTGGCGAGATTGGCACCCT
GCTCCATTGCTGGTGGGATTGCAAGCTGGTGCAGCCGCTTTGGAA
ATCAGTCTGGCGCTTTCTGAGGGACCTCGAGCTTGAGATTCCCTT
CGATCCCGCAATTCCCTTGCTCGGAATCTATCCTAACGAATACAA
GAGCTGTTGTTACAAGGATACGTGTACCCGGATGTTCATCGCGGC
CTTGTTTACGATAGCTAAGACGTGGAATCAGCCTAAGTGCCCCAC
AATGATCGATTGGATCAAGAAAATGTGGCATATTTATACCATGGA
GTATTACGCAGCAATTAAGAATGACGAATTTATTTCCTTCGTTGG
GACCTGGATGAAGCTGGAGACTATTATTCTGAGCAAGCTGTCTCA
GGAGCAAAAGACAAAGCATAGAATCTTCTCTCTCATTGGTGGTAA
CGACTACAAAGACGATGACGACAAGTAAAGCGCTTCTAGAAGTTG
TCTCCTCCTGCACTGACTGACTGATACAATCGATTTCTGGATCCG
CAGGCCTAATCAACCTCTGGATTACAAAATTTGTGAAAGATTGAC
TGGTATTCTTAACTATGTTGCTCCTTTTACGCTATGTGGATACGC
TGCTTTAATGCCTTTGTATCATGCTATTGCTTCCCGTATGGCTTT
CATTTTCTCCTCCTTGTATAAATCCTGGTTGCTGTCTCTTTATGA
GGAGTTGTGGCCCGTTGTCAGGCAACGTGGCGTGGTGTGCACTGT
GTTTGCTGACGCAACCCCCACTGGTTGGGGCATTGCCACCACCTG
TCAGCTCCTTTCCGGGACTTTCGCTTTCCCCCTCCCTATTGCCAC
GGCGGAACTCATCGCCGCCTGCCTTGCCCGCTGCTGGACAGGGGC
TCGGCTGTTGGGCACTGACAATTCCGTGGTGTTGTCGGGGAAGCT
GACGTCCTTTCCATGGCTGCTCGCCTGTGTTGCCACCTGGATTCT
GCGCGGGACGTCCTTCTGCTACGTCCCTTCGGCCCTCAATCCAGC
GGACCTTCCTTCCCGCGAACAAACGACCCAACACCCGTGCGTTTT
ATTCTGTCTTTTTATTGCCGATCCCCTCAGAAGAACTCGTCAAGA
AGGCGATAGAAGGCGATGCGCTGCGAATCGGGAGCGGCGATACCG
TAAAGCACGAGGAAGCGGTCAGCCCATTCGCCGCCAAGCTCTTCA
GCAATATCACGGGTAGCCAACGCTATGTCCTGATAGCGGTCGGCC
GCTTTACTTGTACAGCTCGTCCATGCCGAGAGTGATCCCGGCGGC
GGTCACGAACTCCAGCAGGACCATGTGATCGCGCTTCTCGTTGGG
GTCTTTGCTCAGGGCGGACTGGGTGCTCAGGTAGTGGTTGTCGGG
CAGCAGCACGGGGCCGTCGCCGATGGGGGTGTTCTGCTGGTAGTG
GTCGGCCAGGTGAGTCCAGGAGATGTTTCAGCACTGTTGCCTTTA
GTCTCGAGGCAACTTAGACAACTGAGTATTGATCTGAGCACAGCA
GGGTGTGAGCTGTTTGAAGATACTGGGGTTGGGGGTGAAGAAACT
GCAGAGGACTAACTGGGCTGAGACCCAGTGGCAATGTTTTAGGGC
CTAAGGAATGCCTCTGAAAATCTAGATGGACAACTTTGACTTTGA
GAAAAGAGAGGTGGAAATGAGGAAAATGACTTTTCTTTATTAGAT
TTCGGTAGAAAGAACTTTCATCTTTCCCCTATTTTTGTTATTCGT
TTTAAAACATCTATCTGGAGGCAGGACAAGTATGGTCATTAAAAA
GATGCAGGCAGAAGGCATATATTGGCTCAGTCAAAGTGGGGAACT
TTGGTGGCCAAACATACATTGCTAAGGCTATTCCTATATCAGCTG
GACACATATAAAATGCTGCTAATGCTTCATTACAAACTTATATCC
TTTAATTCCAGATGGGGGCAAAGTATGTCCAGGGGTGAGGAACAA
TTGAAACATTTGGGCTGGAGTAGATTTTGAAAGTCAGCTCTGTGT
GTGTGTGTGTGTGTGTGTGTGTGAGAGCGTGTGTTTCTTTTAACG
TTTTCAGCCTACAGCATACAGGGTTCATGGTGGCAAGAAGATAAC
AAGATTTAAATTATGGCCAGTGACTAGTGCTGCAAGAAGAACAAC
TACCTGCATTTAATGGGAAAGCAAAATCTCAGGCTTTGAGGGAAG
TTAACATAGGCTTGATTCTGGGTGGAAGCTGGGTGTGTAGTTATC
TGGAGGCCAGGCTGGAGCTCTCAGCTCACTATGGGTTCATCTTTA
TTGTCTCCTTTCATCTCAACAGCTGCACGCTGCCGTCCTCGATGT
TGTGGCGGATCTTGAAGTTCACCTTGATGCCGTTCTTCTGCTTGT
CGGCCATGATATAGACGTTGTGGCTGTTGTAGTTGTACTCCAGCT
TGTGCCCCAGGATGTTGCCGTCCTCCTTGAAGTCGATGCCCTTCA
GCTCGATGCGGTTCACCAGGGTGTCGCCCTCGAACTTCACCTCGG
CGCGGGTCTTGTAGTTGCCGTCGTCCTTGAAGAAGATGGTGCGCT
CCTGGACGTAGCCTTCGGGCATGGCGGACTTGAAGAAGTCGTGCT
GCTTCATGTGGTCGGGGTAGCGGCTGAAGCACTGCACGCCGTAGG
TCAGGGTGGTCACGAGGGTGGGCCAGGGCACGGGCAGCTTGCCGG
TGGTGCAGATGAACTTCAGGGTCAGCTTGCCGTAGGTGGCATCGC
CCTCGCCCTCGCCGGACACGCTGAACTTGTGGCCGTTTACGTCGC
CGTCCAGCTCGACCAGGATGGGCACCACCCCGGTGAACAGCTCCT
CGCCCTTGCTCACCATGGTGGCGAATTCGAAGCTTGAGCACGAGA
TCTGAGTCCGGTAGGCCTAGCGGATCTGACGGTTCACTAAACCAG
CTCTGCTTATATAGACCTCCCACCGTACACGCCTACCGCCCATTT
GCGTCAATGGGGCGGAGTTGTTACGACATTTTGGAAAGTCCCGTT
GATTTTGGTGCCAAAACAAACTCCCATTGACGTCAATGGGGTGGA
GACTTGGAAATCCCCGTGAGTCAAACCGCTATCCACGCCCATTGA
TGTACTGCCAAAACCGCATCACCATGGTAATAGCGATGACTAATA
CGTAGATGTACTGCCAAGTAGGAAAGTCCCATAAGGTCATGTACT
GGGCATAATGCCAGGCGGGCCATTTACCGTCATTGACGTCAATAG
GGGGCGTACTTGGCATATGATACACTTGATGTACTGCCAAGTGGG
CAGTTTACCGTAAATACTCCACCCATTGACGTCAATGGAAAGTCC
CTATTGGCGTTACTATGGGAACATACGTCATTATTGACGTCAATG
GGCGGGGGTCGTTGGGCGGTCAGCCAGGCGGGCCATTTACCGTAA
GTTATGTAACGGGCCTGCTGCCGGCTCTGCGGCCTCTTCCGCGTC
TTCGCCTTCGCCCTCAGACGAGTCGGATCTCCCTTTGGGCCGCCT
CCCCGCCTGTCTAGCTTGACTGACTGAGATACAGCGTACCTTCAG
CTCACAGACATGATAAGATACATTGATGAGTTTGGACAAACCACA
ACTAGAATGCAGTGAAAAAAATGCTTTATTTGTGAAATTTGTGAT
GCTATTGCTTTATTTGTAACCATTATAAGCTGCAATAAACAAGTT
AACAACAACAATTGCATTCATTTTATGTTTCAGGTTCAGGGGGAG
GTGTGGGAGGTTTTTTAAAGCAAGTAAAACCTCTACAAATGTGGT
ATTGGCCCATCTCTATCGGTATCGTAGCATAACCCCTTGGGGCCT
CTAAACGGGTCTTGAGGGGTTTTTTGTGCCCCTCGGGCCGGATTG
CTATCTACCGGCATTGGCGCAGAAAAAAATGCCTGATGCGACGCT
GCGCGTCTTATACTCCCACATATGCCAGATTCAGCAACGGATACG
GCTTCCCCAACTTGCCCACTTCCATACGTGTCCTCCTTACCAGAA
ATTTATCCTTAAGGTCGTCAGCTATCCTGCAGGCGATCTCTCGAT
TTCGATCAAGACATTCCTTTAATGGTCTTTTCTGGACACCACTAG
GGGTCAGAAGTAGTTCATCAAACTTTCTTCCCTCCCTAATCTCAT
TGGTTACCTTGGGCTATCGAAACTTAATTAAGCGATCTGCATCTC
AATTAGTCAGCAACCATAGTCCCGCCCCTAACTCCGCCCATCCCG
CCCCTAACTCCGCCCAGTTCCGCCCATTCTCCGCCCCATCGCTGA
CTAATTTTTTTTATTTATGCAGAGGCCGAGGCCGCCTCGGCCTCT
GAGCTATTCCAGAAGTAGTGAGGAGGCTTTTTTGGAGGCCTAGGC
TTTTGCAAAGGAGGTAGCCAACATGATTGAACAAGATGGATTGCA
CGCAGGTTCTCCCGCCGCTTGGGTGGAGAGGCTATTCGGCTATGA
CTGGGCACAACAGACAATCGGCTGCTCTGATGCCGCCGTGTTCCG
GCTGTCAGCGCAGGGGCGCCCGGTTCTTTTTGTCAAGACCGACCT
GTCCGGTGCCCTGAATGAACTCCAGGACGAGGCAGCGCGGCTATC
GTGGCTGGCCACGACGGGCGTTCCTTGCGCAGCTGTGCTCGACGT
TGTCACTGAAGCGGGAAGGGACTGGCTGCTATTGGGCGAAGTGCC
GGGGCAGGATCTCCTGTCATCTCACCTTGCTCCTGCCGAGAAAGT
ATCCATCATGGCTGATGCAATGCGGCGGCTGCATACGCTTGATCC
GGCTACCTGCCCATTCGACCACCAAGCGAAACATCGCATCGAGCG
AGCACGTACTCGGATGGAAGCCGGTCTTGTCGATCAGGATGATCT
GGACGAAGAGCATCAGGGGCTCGCGCCAGCCGAACTGTTCGCCAG
GCTCAAGGCGCGGATGCCCGACGGCGAGGATCTCGTCGTGACCCA
CGGCGATGCCTGCTTGCCGAATATCATGGTGGAAAATGGCCGCTT
TTCTGGATTCATCGACTGTGGCCGGCTGGGTGTGGCGGACCGCTA
TCAGGACATAGCGTTGGCTACCCGTGATATTGCTGAAGAGCTTGG
CGGCGAATGGGCTGACCGCTTCCTCGTGCTTTACGGTATCGCCGC
TCCCGATTCGCAGCGCATCGCCTTCTATCGCCTTCTTGACGAGTT
CTTCTAGTATGTAAGCCCTGTGCCTTCTAGTTGCCAGCCATCTGT
TGTTTGCCCCTCCCCCGTGCCTTCCTTGACCCTGGAAGGTGCCAC
TCCCACTGTCCTTTCCTAATAAAATGAGGAAATTGCATCGCATTG
TCTGAGTAGGTGTCATTCTATTCTGGGGGGTGGGGTGGGGCAGGA
CAGCAAGGGGGAGGATTGGGAAGACAATAGCAGGCATGCTGGGGA
TGCGGTGGGCTCTATGGTTAATTAACCAGTCAAGTCAGCTACTTG
GCGAGATCGACTTGTCTGGGTTTCGACTACGCTCAGAATTGCGTC
AGTCAAGTTCGATCTGGTCCTTGCTATTGCACCCGTTCTCCGATT
ACGAGTTTCATTTAAATCATGTGAGCAAAAGGCCAGCAAAAGGCC
AGGAACCGTAAAAAGGCCGCGTTGCTGGCGTTTTTCCATAGGCTC
CGCCCCCCTGACGAGCATCACAAAAATCGACGCTCAAGTCAGAGG
TGGCGAAACCCGACAGGACTATAAAGATACCAGGCGTTTCCCCCT
GGAAGCTCCCTCGTGCGCTCTCCTGTTCCGACCCTGCCGCTTACC
GGATACCTGTCCGCCTTTCTCCCTTCGGGAAGCGTGGCGCTTTCT
CATAGCTCACGCTGTAGGTATCTCAGTTCGGTGTAGGTCGTTCGC
TCCAAGCTGGGCTGTGTGCACGAACCCCCCGTTCAGCCCGACCGC
TGCGCCTTATCCGGTAACTATCGTCTTGAGTCCAACCCGGTAAGA
CACGACTTATCGCCACTGGCAGCAGCCACTGGTAACAGGATTAGC
AGAGCGAGGTATGTAGGCGGTGCTACAGAGTTCTTGAAGTGGTGG
CCTAACTACGGCTACACTAGAAGAACAGTATTTGGTATCTGCGCT
CTGCTGAAGCCAGTTACCTTCGGAAAAAGAGTTGGTAGCTCTTGA
TCCGGCAAACAAACCACCGCTGGTAGCGGTGGTTTTTTTGTTTGC
AAGCAGCAGATTACGCGCAGAAAAAAAGGATCTCAAGAAGATCCT
TTGATCTTTTCTACGGGGTCTGACGCTCAGTGGAACGAAAACTCA
CGTTAAGGGATTTTGGTCATGAGATTATCAAAAAGGATCTTCACC
TAGATCCTTTTAAATTAAAAATGAAGTTTTAAATCAATCTAAAGT
ATATATGAGTAAACTTGGTCTGACAGTTACCAATGCTTAATCAGT
GAGGCACCTATCTCAGCGATCTGTCTATTTCGTTCATCCATAGTT
GCATTTAAATTTCCGAACTCTCCAAGGCCCTCGTCGGAAAATCTT
CAAACCTTTCGTCCGATCCATCTTGCAGGCTACCTCTCGAACGAA
CTATCGCAAGTCTCTTGGCCGGCCTTGCGCCTTGGCTATTGCTTG
GCAGCGCCTATCGCCAGGTATTACTCCAATCCCGAATATCCGAGA
TCGGGATCACCCGAGAGAAGTTCAACCTACATCCTCAATCCCGAT
CTATCCGAGATCCGAGGAATATCGAAATCGGGGCGCGCCTGGTGT
ACCGAGAACGATCCTCTCAGTGCGAGTCTCGACGATCCATATCGT
TGCTTGGCAGTCAGCCAGTCGGAATCCAGCTTGGGACCCAGGAAG
TCCAATCGTCAGATATTGTACTCAAGCCTGGTCACGGCAGCGTAC
CGATCTGTTTAAACCTAGATATTGATAGTCTGATCGGTCAACGTA
TAATCGAGTCCTAGCTTTTGCAAACATCTATCAAGAGACAGGATC
AGCAGGAGGCTTTCGCATGAGTATTCAACATTTCCGTGTCGCCCT
TATTCCCTTTTTTGCGGCATTTTGCCTTCCTGTTTTTGCTCACCC
AGAAACGCTGGTGAAAGTAAAAGATGCTGAAGATCAGTTGGGTGC
GCGAGTGGGTTACATCGAACTGGATCTCAACAGCGGTAAGATCCT
TGAGAGTTTTCGCCCCGAAGAACGCTTTCCAATGATGAGCACTTT
TAAAGTTCTGCTATGTGGCGCGGTATTATCCCGTATTGACGCCGG
GCAAGAGCAACTCGGTCGCCGCATACACTATTCTCAGAATGACTT
GGTTGAGTATTCACCAGTCACAGAAAAGCATCTTACGGATGGCAT
GACAGTAAGAGAATTATGCAGTGCTGCCATAACCATGAGTGATAA
CACTGCGGCCCGGCCGCAACTTACTTCTGACAACGATTGGAGGAC
CGAAGGAGCTAACCGCTTTTTTGCACAACATGGGGGATCATGTAA
CTCGCCTTGATCGTTGGGAACCGGAGCTGAATGAAGCCATACCAA
ACGACGAGCGTGACACCACGATGCCTGTAGCAATGGCAACAACCT
TGCGTAAACTATTAACTGGCGAACTACTTACTCTAGCTTCCCGGC
AACAGTTGATAGACTGGATGGAGGCGGATAAAGTTGCAGGACCAC
TTCTGCGCTCGGCCCTTCCGGCTGGCTGGTTTATTGCTGATAAAT
CTGGAGCCGGTGAGCGTGGGTCTCGCGGTATCATTGCAGCACTGG
GGCCAGATGGTAAGCCCTCCCGTATCGTAGTTATCTACACGACGG
GGAGTCAGGCAACTATGGATGAACGAAATAGACAGATCGCTGAGA
TAGGTGCCTCACTGATTAAGCATTGGTAACCGATTCTAGGTGCAT
TGGCGCAGAAAAAAATGCCTGATGCGACGCTGCGCGTCTTATACT
CCCACATATGCCAGATTCAGCAACGGATACGGCTTCCCCAACTTG
CCCACTTCCATACGTGTCCTCCTTACCAGAAATTTATCCTTAAGA
TCGTTTAAACTCGACTCTGGCTCTATCGAATCTCCGTCGTTTCGA
GCTTACGCGAACAGCCGTGGCGCTCATTTGCTCGTCGGGCATCGA
ATCTCGTCAGCTATCGTCAGCTTACCTTTTTGGCAGCGATCGCGG
CTCCCGACATCTTGGACCATTAGCTCCACAGGTATCTTCTTCCCT
CTAGTGGTCATAACAGCAGCTTCAGCTACCTCTCAATTCAAAAAA
CCCCTCAAGACCCGTTTAGAGGCCCCAAGGGGTTATGCTATCAAT
CGTTGCGTTACACACACAAAAAACCAACACACATCCATCTTCGAT
GGATAGCGATTTTATTATCTAACTGCTGATCGAGTGTAGCCAGAT
CTAGTAATCAATTACGGGGTCATTAGTTCATAGCCCATATATGGA
GTTCCGCGTTACATAACTTACGGTAAATGGCCCGCCTGGCTGACC
GCCCAACGACCCCCGCCCATTGACGTCAATAATGACGTATGTTCC
CATAGTAACGCCAATAGGGACTTTCCATTGACGTCAATGGGTGGA
GTATTTACGGTAAACTGCCCACTTGGCAGTACATCAAGTGTATCA
TATGCCAAGTACGCCCCCTATTGACGTCAATGACGGTAAATGGCC
CGCCTGGCATTATGCCCAGTACATGACCTTATGGGACTTTCCTAC
TTGGCAGTACATCTACGTATTAGTCATCGCTATTACCATGCTGAT
GCGGTTTTGGCAGTACATCAATGGGCGTGGATAGCGGTTTGACTC
ACGGGGATTTCCAAGTCTCCACCCCATTGACGTCAATGGGAGTTT
GTTTTGGCACCAAAATCAACGGGACTTTCCAAAATGTCGTAACAA
CTCCGCCCCATTGACGCAAATGGGCGGTAGGCGTGTACGGTGGGA
GGTCTATATAAGCAGAGCTGGTTTAGTGAACCGTCAGATCAGATC
TTTGTCGATCCTACCATCCACTCGACACACCCGCCAG
(SEQ ID NO: 44)
LINE-1 Plasmid EV71 IRES (SEQ ID NO: 45)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGTGAGACAGTTAAAACAGCTGTGGGTTGTCAC
CCACCCACAGGGTCCACTGGGCGCTAGTACACTGGTATCTCGGTA
CCTTTGTACGCCTGTTTTATACCCCCTCCCTGATTTGCAACTTAG
AAGCAACGCAAACCAGATCAATAGTAGGTGTGACATACCAGTCGC
ATCTTGATCAAGCACTTCTGTATCCCCGGACCGAGTATCAATAGA
CTGTGCACACGGTTGAAGGAGAAAACGTCCGTTACCCGGCTAACT
ACTTCGAGAAGCCTAGTAACGCCATTGAAGTTGCAGAGTGTTTCG
CTCAGCACTCCCCCCGTGTAGATCAGGTCGATGAGTCACCGCATT
CCCCACGGGCGACCGTGGCGGTGGCTGCGTTGGCGGCCTGCCTAT
GGGGTAACCCATAGGACGCTCTAATACGGACATGGCGTGAAGAGT
CTATTGAGCTAGTTAGTAGTCCTCCGGCCCCTGAATGCGGCTAAT
CCTAACTGCGGAGCACATACCCTTAATCCAAAGGGCAGTGTGTCG
TAACGGGCAACTCTGCAGCGGAACCGACTACTTTGGGTGTCCGTG
TTTCTTTTTATTCTTGTATTGGCTGCTTATGGTGACAATTAAAGA
ATTGTTACCATATAGCTATTGGATTGGCCATCCAGTGTCAAACAG
AGCTATTGTATATCTCTTTGTTGGATTCACACCTCTCACTCTTGA
AACGTTACACACCCTCAATTACATTATACTGCTGAACACGAAGCG
TACATGACCGGCTCTAACTCACATATCACCATCCTTACACTTAAC
ATTAACGGCCTCAACTCAGCTATCAAGCGCCATCGGCTGGCCAGC
TGGATCAAATCACAGGATCCAAGCGTTTGTTGCATCCAAGAGACC
CACCTGACCTGTAGAGATACTCACCGCCTCAAGATCAAGGGATGG
CGAAAGATTTATCAGGCGAACGGTAAGCAGAAGAAAGCCGGAGTC
GCAATTCTGGTCTCAGACAAGACGGATTTCAAGCCCACCAAAATT
AAGCGTGATAAGGAAGGTCACTATATTATGGTGAAAGGCAGCATA
CAGCAGGAAGAACTTACCATATTGAACATCTACGCGCCAAACACC
GGCGCACCTCGCTTTATCAAACAGGTCCTGTCCGATCTGCAGCGA
GATCTGGATTCTCATACGTTGATTATGGGTGATTTCAATACACCA
TTGAGCACCCTGGATCGCAGCACCAGGCAAAAGGTAAATAAAGAC
ACGCAAGAGCTCAATAGCGCACTGCATCAGGCAGATCTCATTGAT
ATTTATCGCACTCTTCATCCTAAGAGTACCGAGTACACATTCTTC
AGCGCCCCACATCATACATACTCAAAGATCGATCATATCGTCGGC
TCAAAGGCTCTGCTGTCAAAGTGCAAGCGCACAGAGATAATTACA
AATTACCTGTCAGATCATAGCGCGATCAAGCTCGAGCTGAGAATC
AAGAACCTGACCCAGAGCCGGAGTACCACTTGGAAGCTTAATAAC
CTGCTGCTCAACGATTATTGGGTCCACAATGAGATGAAGGCAGAG
ATTAAAATGTTCTTCGAAACAAATGAGAATAAGGATACTACCTAT
CAAAACCTTTGGGATGCCTTTAAGGCCGTCTGCAGAGGCAAGTTC
ATCGCCCTCAACGCCTATAAAAGAAAACAAGAGAGATCTAAGATC
GATACTCTCACCTCTCAGCTGAAGGAGTTGGAGAAACAGGAACAG
ACCCACTCCAAGGCGTCAAGACGGCAGGAGATCACAAAGATTCGC
GCCGAGTTGAAAGAGATCGAAACCCAAAAGACTCTTCAGAAAATT
AACGAGTCTCGTAGTTGGTTCTTCGAGCGGATTAATAAGATAGAC
AGACCTCTGGCACGACTGATTAAGAAGAAGCGCGAAAAGAACCAG
ATTGATACCATCAAGAACGACAAGGGCGACATCACTACTGACCCG
ACCGAGATCCAGACCACTATTCGGGAGTATTATAAGCATTTGTAT
GCTAACAAGCTTGAGAACCTGGAAGAGATGGACACTTTTCTGGAT
ACCTATACTCTGCCACGGCTTAATCAAGAGGAAGTCGAGTCCCTC
AACCGCCCAATTACAGGAAGCGAGATTGTGGCCATAATTAACTCC
CTGCCGACAAAGAAATCTCCTGGTCCGGACGGGTTTACAGCTGAG
TTTTATCAACGGTATATGGAAGAGCTTGTACCGTTTCTGCTCAAG
CTCTTTCAGTCTATAGAAAAGGAAGGCATCTTGCCCAATTCCTTC
TACGAAGCTTCTATAATACTTATTCCCAAACCAGGACGCGATACC
ACAAAGAAGGAAAACTTCCGGCCCATTAGTCTCATGAATATCGAC
GCTAAAATATTGAACAAGATTCTCGCCAACAGAATCCAACAACAT
ATTAAGAAATTGATACATCACGACCAGGTGGGGTTTATACCTGGC
ATGCAGGGCTGGTTTAACATCCGGAAGAGTATTAACGTCATTCAA
CACATTAATAGAGCTAAGGATAAGAATCATATGATCATCTCTATA
GACGCGGAAAAGGCATTCGATAAGATTCAGCAGCCATTTATGCTC
AAGACTCTGAACAAACTCGGCATCGACGGAACATATTTTAAGATT
ATTCGCGCAATTTACGATAAGCCGACTGCTAACATTATCCTTAAC
GGCCAAAAGCTCGAGGCCTTTCCGCTCAAGACTGGAACCCGCCAA
GGCTGTCCCCTCTCCCCGCTTTTGTTTAATATTGTACTCGAGGTG
CTGGCTAGGGCTATTCGTCAAGAGAAAGAGATTAAAGGGATACAG
CTCGGGAAGGAAGAGGTCAAGCTTTCCTTGTTCGCCGATGATATG
ATTGTGTACCTGGAGAATCCTATTGTGTCTGCTCAGAACCTTCTT
AAACTTATTTCTAACTTTAGCAAGGTCAGCGGCTATAAGATTAAC
GTCCAGAAATCTCAGGCCTTTCTGTACACAAATAATCGACAGACC
GAATCCCAGATAATGGGTGAGCTTCCGTTTGTCATAGCCAGCAAA
AGGATAAAGTATCTCGGAATCCAGCTGACACGAGACGTTAAAGAT
TTGTTTAAGGAAAATTACAAGCCTCTCCTGAAAGAGATTAAGGAA
GATACTAATAAGTGGAAGAATATCCCCTGTTCATGGGTTGGCAGA
ATCAACATAGTGAAGATGGCAATACTTCCTAAAGTGATATATCGC
TTTAACGCCATCCCAATTAAACTGCCTATGACCTTCTTTACGGAG
CTCGAGAAAACAACCCTTAAATTTATATGGAATCAAAAGAGAGCA
AGAATAGCGAAGTCCATCTTGAGCCAGAAGAATAAGGCCGGTGGG
ATTACTTTGCCTGATTTTAAGTTGTATTATAAAGCCACAGTAACT
AAGACAGCCTGGTATTGGTATCAGAATAGAGACATCGACCAGTGG
AATCGGACCGAACCATCAGAGATAATGCCCCACATCTATAATTAC
CTTATATTCGATAAGCCAGAAAAGAATAAACAGTGGGGCAAAGAC
AGCCTCTTCAACAAGTGGTGTTGGGAGAATTGGCTGGCCATATGC
CGGAAACTCAAGCTCGACCCCTTTCTTACACCCTACACTAAAATC
AACAGTAGGTGGATCAAGGACTTGAATGTCAAGCCAAAGACTATA
AAGACACTGGAAGAGAATCTTGGGATCACAATACAAGATATAGGC
GTCGGCAAAGATTTTATGTCAAAGACGCCCAAGGCCATGGCCACT
AAGGATAAGATTGATAAGTGGGACCTTATTAAGCTCAAAAGCTTC
TGTACTGCCAAGGAGACCACGATCAGAGTTAATAGGCAGCCCACT
ACATGGGAAAAGATTTTCGCCACTTATTCATCAGATAAGGGGTTG
ATAAGCAGAATATATAACGAGCTGAAGCAGATCTACAAGAAGAAA
ACGAATAATCCCATCAAGAAGTGGGCAAAAGATATGAACAGGCAT
TTTAGCAAAGAGGATATCTACGCCGCGAAGAAGCATATGAAGAAG
TGTAGTTCAAGCTTGGCCATTCGTGAGATGCAGATTAAGACGACC
ATGCGATACCACCTTACCCCAGTGAGGATGGCAATTATCAAGAAA
TCTGGCAATAATAGATGTTGGCGGGGCTGTGGCGAGATTGGCACC
CTGCTCCATTGCTGGTGGGATTGCAAGCTGGTGCAGCCGCTTTGG
AAATCAGTCTGGCGCTTTCTGAGGGACCTCGAGCTTGAGATTCCC
TTCGATCCCGCAATTCCCTTGCTCGGAATCTATCCTAACGAATAC
AAGAGCTGTTGTTACAAGGATACGTGTACCCGGATGTTCATCGCG
GCCTTGTTTACGATAGCTAAGACGTGGAATCAGCCTAAGTGCCCC
ACAATGATCGATTGGATCAAGAAAATGTGGCATATTTATACCATG
GAGTATTACGCAGCAATTAAGAATGACGAATTTATTTCCTTCGTT
GGGACCTGGATGAAGCTGGAGACTATTATTCTGAGCAAGCTGTCT
CAGGAGCAAAAGACAAAGCATAGAATCTTCTCTCTCATTGGTGGT
AACGACTACAAAGACGATGACGACAAGTAAAGCGCTTCTAGAAGT
TGTCTCCTCCTGCACTGACTGACTGATACAATCGATTTCTGGATC
CGCAGGCCTAATCAACCTCTGGATTACAAAATTTGTGAAAGATTG
ACTGGTATTCTTAACTATGTTGCTCCTTTTACGCTATGTGGATAC
GCTGCTTTAATGCCTTTGTATCATGCTATTGCTTCCCGTATGGCT
TTCATTTTCTCCTCCTTGTATAAATCCTGGTTGCTGTCTCTTTAT
GAGGAGTTGTGGCCCGTTGTCAGGCAACGTGGCGTGGTGTGCACT
GTGTTTGCTGACGCAACCCCCACTGGTTGGGGCATTGCCACCACC
TGTCAGCTCCTTTCCGGGACTTTCGCTTTCCCCCTCCCTATTGCC
ACGGCGGAACTCATCGCCGCCTGCCTTGCCCGCTGCTGGACAGGG
GCTCGGCTGTTGGGCACTGACAATTCCGTGGTGTTGTCGGGGAAG
CTGACGTCCTTTCCATGGCTGCTCGCCTGTGTTGCCACCTGGATT
CTGCGCGGGACGTCCTTCTGCTACGTCCCTTCGGCCCTCAATCCA
GCGGACCTTCCTTCCCGCGAACAAACGACCCAACACCCGTGCGTT
TTATTCTGTCTTTTTATTGCCGATCCCCTCAGAAGAACTCGTCAA
GAAGGCGATAGAAGGCGATGCGCTGCGAATCGGGAGCGGCGATAC
CGTAAAGCACGAGGAAGCGGTCAGCCCATTCGCCGCCAAGCTCTT
CAGCAATATCACGGGTAGCCAACGCTATGTCCTGATAGCGGTCGG
CCGCTTTACTTGTACAGCTCGTCCATGCCGAGAGTGATCCCGGCG
GCGGTCACGAACTCCAGCAGGACCATGTGATCGCGCTTCTCGTTG
GGGTCTTTGCTCAGGGCGGACTGGGTGCTCAGGTAGTGGTTGTCG
GGCAGCAGCACGGGGCCGTCGCCGATGGGGGTGTTCTGCTGGTAG
TGGTCGGCCAGGTGAGTCCAGGAGATGTTTCAGCACTGTTGCCTT
TAGTCTCGAGGCAACTTAGACAACTGAGTATTGATCTGAGCACAG
CAGGGTGTGAGCTGTTTGAAGATACTGGGGTTGGGGGTGAAGAAA
CTGCAGAGGACTAACTGGGCTGAGACCCAGTGGCAATGTTTTAGG
GCCTAAGGAATGCCTCTGAAAATCTAGATGGACAACTTTGACTTT
GAGAAAAGAGAGGTGGAAATGAGGAAAATGACTTTTCTTTATTAG
ATTTCGGTAGAAAGAACTTTCATCTTTCCCCTATTTTTGTTATTC
GTTTTAAAACATCTATCTGGAGGCAGGACAAGTATGGTCATTAAA
AAGATGCAGGCAGAAGGCATATATTGGCTCAGTCAAAGTGGGGAA
CTTTGGTGGCCAAACATACATTGCTAAGGCTATTCCTATATCAGC
TGGACACATATAAAATGCTGCTAATGCTTCATTACAAACTTATAT
CCTTTAATTCCAGATGGGGGCAAAGTATGTCCAGGGGTGAGGAAC
AATTGAAACATTTGGGCTGGAGTAGATTTTGAAAGTCAGCTCTGT
GTGTGTGTGTGTGTGTGTGTGTGTGAGAGCGTGTGTTTCTTTTAA
CGTTTTCAGCCTACAGCATACAGGGTTCATGGTGGCAAGAAGATA
ACAAGATTTAAATTATGGCCAGTGACTAGTGCTGCAAGAAGAACA
ACTACCTGCATTTAATGGGAAAGCAAAATCTCAGGCTTTGAGGGA
AGTTAACATAGGCTTGATTCTGGGTGGAAGCTGGGTGTGTAGTTA
TCTGGAGGCCAGGCTGGAGCTCTCAGCTCACTATGGGTTCATCTT
TATTGTCTCCTTTCATCTCAACAGCTGCACGCTGCCGTCCTCGAT
GTTGTGGCGGATCTTGAAGTTCACCTTGATGCCGTTCTTCTGCTT
GTCGGCCATGATATAGACGTTGTGGCTGTTGTAGTTGTACTCCAG
CTTGTGCCCCAGGATGTTGCCGTCCTCCTTGAAGTCGATGCCCTT
CAGCTCGATGCGGTTCACCAGGGTGTCGCCCTCGAACTTCACCTC
GGCGCGGGTCTTGTAGTTGCCGTCGTCCTTGAAGAAGATGGTGCG
CTCCTGGACGTAGCCTTCGGGCATGGCGGACTTGAAGAAGTCGTG
CTGCTTCATGTGGTCGGGGTAGCGGCTGAAGCACTGCACGCCGTA
GGTCAGGGTGGTCACGAGGGTGGGCCAGGGCACGGGCAGCTTGCC
GGTGGTGCAGATGAACTTCAGGGTCAGCTTGCCGTAGGTGGCATC
GCCCTCGCCCTCGCCGGACACGCTGAACTTGTGGCCGTTTACGTC
GCCGTCCAGCTCGACCAGGATGGGCACCACCCCGGTGAACAGCTC
CTCGCCCTTGCTCACCATGGTGGCGAATTCGAAGCTTGAGCACGA
GATCTGAGTCCGGTAGGCCTAGCGGATCTGACGGTTCACTAAACC
AGCTCTGCTTATATAGACCTCCCACCGTACACGCCTACCGCCCAT
TTGCGTCAATGGGGCGGAGTTGTTACGACATTTTGGAAAGTCCCG
TTGATTTTGGTGCCAAAACAAACTCCCATTGACGTCAATGGGGTG
GAGACTTGGAAATCCCCGTGAGTCAAACCGCTATCCACGCCCATT
GATGTACTGCCAAAACCGCATCACCATGGTAATAGCGATGACTAA
TACGTAGATGTACTGCCAAGTAGGAAAGTCCCATAAGGTCATGTA
CTGGGCATAATGCCAGGCGGGCCATTTACCGTCATTGACGTCAAT
AGGGGGCGTACTTGGCATATGATACACTTGATGTACTGCCAAGTG
GGCAGTTTACCGTAAATACTCCACCCATTGACGTCAATGGAAAGT
CCCTATTGGCGTTACTATGGGAACATACGTCATTATTGACGTCAA
TGGGCGGGGGTCGTTGGGCGGTCAGCCAGGCGGGCCATTTACCGT
AAGTTATGTAACGGGCCTGCTGCCGGCTCTGCGGCCTCTTCCGCG
TCTTCGCCTTCGCCCTCAGACGAGTCGGATCTCCCTTTGGGCCGC
CTCCCCGCCTGTCTAGCTTGACTGACTGAGATACAGCGTACCTTC
AGCTCACAGACATGATAAGATACATTGATGAGTTTGGACAAACCA
CAACTAGAATGCAGTGAAAAAAATGCTTTATTTGTGAAATTTGTG
ATGCTATTGCTTTATTTGTAACCATTATAAGCTGCAATAAACAAG
TTAACAACAACAATTGCATTCATTTTATGTTTCAGGTTCAGGGGG
AGGTGTGGGAGGTTTTTTAAAGCAAGTAAAACCTCTACAAATGTG
GTATTGGCCCATCTCTATCGGTATCGTAGCATAACCCCTTGGGGC
CTCTAAACGGGTCTTGAGGGGTTTTTTGTGCCCCTCGGGCCGGAT
TGCTATCTACCGGCATTGGCGCAGAAAAAAATGCCTGATGCGACG
CTGCGCGTCTTATACTCCCACATATGCCAGATTCAGCAACGGATA
CGGCTTCCCCAACTTGCCCACTTCCATACGTGTCCTCCTTACCAG
AAATTTATCCTTAAGGTCGTCAGCTATCCTGCAGGCGATCTCTCG
ATTTCGATCAAGACATTCCTTTAATGGTCTTTTCTGGACACCACT
AGGGGTCAGAAGTAGTTCATCAAACTTTCTTCCCTCCCTAATCTC
ATTGGTTACCTTGGGCTATCGAAACTTAATTAAGCGATCTGCATC
TCAATTAGTCAGCAACCATAGTCCCGCCCCTAACTCCGCCCATCC
CGCCCCTAACTCCGCCCAGTTCCGCCCATTCTCCGCCCCATCGCT
GACTAATTTTTTTTATTTATGCAGAGGCCGAGGCCGCCTCGGCCT
CTGAGCTATTCCAGAAGTAGTGAGGAGGCTTTTTTGGAGGCCTAG
GCTTTTGCAAAGGAGGTAGCCAACATGATTGAACAAGATGGATTG
CACGCAGGTTCTCCCGCCGCTTGGGTGGAGAGGCTATTCGGCTAT
GACTGGGCACAACAGACAATCGGCTGCTCTGATGCCGCCGTGTTC
CGGCTGTCAGCGCAGGGGCGCCCGGTTCTTTTTGTCAAGACCGAC
CTGTCCGGTGCCCTGAATGAACTCCAGGACGAGGCAGCGCGGCTA
TCGTGGCTGGCCACGACGGGCGTTCCTTGCGCAGCTGTGCTCGAC
GTTGTCACTGAAGCGGGAAGGGACTGGCTGCTATTGGGCGAAGTG
CCGGGGCAGGATCTCCTGTCATCTCACCTTGCTCCTGCCGAGAAA
GTATCCATCATGGCTGATGCAATGCGGCGGCTGCATACGCTTGAT
CCGGCTACCTGCCCATTCGACCACCAAGCGAAACATCGCATCGAG
CGAGCACGTACTCGGATGGAAGCCGGTCTTGTCGATCAGGATGAT
CTGGACGAAGAGCATCAGGGGCTCGCGCCAGCCGAACTGTTCGCC
AGGCTCAAGGCGCGGATGCCCGACGGCGAGGATCTCGTCGTGACC
CACGGCGATGCCTGCTTGCCGAATATCATGGTGGAAAATGGCCGC
TTTTCTGGATTCATCGACTGTGGCCGGCTGGGTGTGGCGGACCGC
TATCAGGACATAGCGTTGGCTACCCGTGATATTGCTGAAGAGCTT
GGCGGCGAATGGGCTGACCGCTTCCTCGTGCTTTACGGTATCGCC
GCTCCCGATTCGCAGCGCATCGCCTTCTATCGCCTTCTTGACGAG
TTCTTCTAGTATGTAAGCCCTGTGCCTTCTAGTTGCCAGCCATCT
GTTGTTTGCCCCTCCCCCGTGCCTTCCTTGACCCTGGAAGGTGCC
ACTCCCACTGTCCTTTCCTAATAAAATGAGGAAATTGCATCGCAT
TGTCTGAGTAGGTGTCATTCTATTCTGGGGGGTGGGGTGGGGCAG
GACAGCAAGGGGGAGGATTGGGAAGACAATAGCAGGCATGCTGGG
GATGCGGTGGGCTCTATGGTTAATTAACCAGTCAAGTCAGCTACT
TGGCGAGATCGACTTGTCTGGGTTTCGACTACGCTCAGAATTGCG
TCAGTCAAGTTCGATCTGGTCCTTGCTATTGCACCCGTTCTCCGA
TTACGAGTTTCATTTAAATCATGTGAGCAAAAGGCCAGCAAAAGG
CCAGGAACCGTAAAAAGGCCGCGTTGCTGGCGTTTTTCCATAGGC
TCCGCCCCCCTGACGAGCATCACAAAAATCGACGCTCAAGTCAGA
GGTGGCGAAACCCGACAGGACTATAAAGATACCAGGCGTTTCCCC
CTGGAAGCTCCCTCGTGCGCTCTCCTGTTCCGACCCTGCCGCTTA
CCGGATACCTGTCCGCCTTTCTCCCTTCGGGAAGCGTGGCGCTTT
CTCATAGCTCACGCTGTAGGTATCTCAGTTCGGTGTAGGTCGTTC
GCTCCAAGCTGGGCTGTGTGCACGAACCCCCCGTTCAGCCCGACC
GCTGCGCCTTATCCGGTAACTATCGTCTTGAGTCCAACCCGGTAA
GACACGACTTATCGCCACTGGCAGCAGCCACTGGTAACAGGATTA
GCAGAGCGAGGTATGTAGGCGGTGCTACAGAGTTCTTGAAGTGGT
GGCCTAACTACGGCTACACTAGAAGAACAGTATTTGGTATCTGCG
CTCTGCTGAAGCCAGTTACCTTCGGAAAAAGAGTTGGTAGCTCTT
GATCCGGCAAACAAACCACCGCTGGTAGCGGTGGTTTTTTTGTTT
GCAAGCAGCAGATTACGCGCAGAAAAAAAGGATCTCAAGAAGATC
CTTTGATCTTTTCTACGGGGTCTGACGCTCAGTGGAACGAAAACT
CACGTTAAGGGATTTTGGTCATGAGATTATCAAAAAGGATCTTCA
CCTAGATCCTTTTAAATTAAAAATGAAGTTTTAAATCAATCTAAA
GTATATATGAGTAAACTTGGTCTGACAGTTACCAATGCTTAATCA
GTGAGGCACCTATCTCAGCGATCTGTCTATTTCGTTCATCCATAG
TTGCATTTAAATTTCCGAACTCTCCAAGGCCCTCGTCGGAAAATC
TTCAAACCTTTCGTCCGATCCATCTTGCAGGCTACCTCTCGAACG
AACTATCGCAAGTCTCTTGGCCGGCCTTGCGCCTTGGCTATTGCT
TGGCAGCGCCTATCGCCAGGTATTACTCCAATCCCGAATATCCGA
GATCGGGATCACCCGAGAGAAGTTCAACCTACATCCTCAATCCCG
ATCTATCCGAGATCCGAGGAATATCGAAATCGGGGCGCGCCTGGT
GTACCGAGAACGATCCTCTCAGTGCGAGTCTCGACGATCCATATC
GTTGCTTGGCAGTCAGCCAGTCGGAATCCAGCTTGGGACCCAGGA
AGTCCAATCGTCAGATATTGTACTCAAGCCTGGTCACGGCAGCGT
ACCGATCTGTTTAAACCTAGATATTGATAGTCTGATCGGTCAACG
TATAATCGAGTCCTAGCTTTTGCAAACATCTATCAAGAGACAGGA
TCAGCAGGAGGCTTTCGCATGAGTATTCAACATTTCCGTGTCGCC
CTTATTCCCTTTTTTGCGGCATTTTGCCTTCCTGTTTTTGCTCAC
CCAGAAACGCTGGTGAAAGTAAAAGATGCTGAAGATCAGTTGGGT
GCGCGAGTGGGTTACATCGAACTGGATCTCAACAGCGGTAAGATC
CTTGAGAGTTTTCGCCCCGAAGAACGCTTTCCAATGATGAGCACT
TTTAAAGTTCTGCTATGTGGCGCGGTATTATCCCGTATTGACGCC
GGGCAAGAGCAACTCGGTCGCCGCATACACTATTCTCAGAATGAC
TTGGTTGAGTATTCACCAGTCACAGAAAAGCATCTTACGGATGGC
ATGACAGTAAGAGAATTATGCAGTGCTGCCATAACCATGAGTGAT
AACACTGCGGCCCGGCCGCAACTTACTTCTGACAACGATTGGAGG
ACCGAAGGAGCTAACCGCTTTTTTGCACAACATGGGGGATCATGT
AACTCGCCTTGATCGTTGGGAACCGGAGCTGAATGAAGCCATACC
AAACGACGAGCGTGACACCACGATGCCTGTAGCAATGGCAACAAC
CTTGCGTAAACTATTAACTGGCGAACTACTTACTCTAGCTTCCCG
GCAACAGTTGATAGACTGGATGGAGGCGGATAAAGTTGCAGGACC
ACTTCTGCGCTCGGCCCTTCCGGCTGGCTGGTTTATTGCTGATAA
ATCTGGAGCCGGTGAGCGTGGGTCTCGCGGTATCATTGCAGCACT
GGGGCCAGATGGTAAGCCCTCCCGTATCGTAGTTATCTACACGAC
GGGGAGTCAGGCAACTATGGATGAACGAAATAGACAGATCGCTGA
GATAGGTGCCTCACTGATTAAGCATTGGTAACCGATTCTAGGTGC
ATTGGCGCAGAAAAAAATGCCTGATGCGACGCTGCGCGTCTTATA
CTCCCACATATGCCAGATTCAGCAACGGATACGGCTTCCCCAACT
TGCCCACTTCCATACGTGTCCTCCTTACCAGAAATTTATCCTTAA
GATCGTTTAAACTCGACTCTGGCTCTATCGAATCTCCGTCGTTTC
GAGCTTACGCGAACAGCCGTGGCGCTCATTTGCTCGTCGGGCATC
GAATCTCGTCAGCTATCGTCAGCTTACCTTTTTGGCAGCGATCGC
GGCTCCCGACATCTTGGACCATTAGCTCCACAGGTATCTTCTTCC
CTCTAGTGGTCATAACAGCAGCTTCAGCTACCTCTCAATTCAAAA
AACCCCTCAAGACCCGTTTAGAGGCCCCAAGGGGTTATGCTATCA
ATCGTTGCGTTACACACACAAAAAACCAACACACATCCATCTTCG
ATGGATAGCGATTTTATTATCTAACTGCTGATCGAGTGTAGCCAG
ATCTAGTAATCAATTACGGGGTCATTAGTTCATAGCCCATATATG
GAGTTCCGCGTTACATAACTTACGGTAAATGGCCCGCCTGGCTGA
CCGCCCAACGACCCCCGCCCATTGACGTCAATAATGACGTATGTT
CCCATAGTAACGCCAATAGGGACTTTCCATTGACGTCAATGGGTG
GAGTATTTACGGTAAACTGCCCACTTGGCAGTACATCAAGTGTAT
CATATGCCAAGTACGCCCCCTATTGACGTCAATGACGGTAAATGG
CCCGCCTGGCATTATGCCCAGTACATGACCTTATGGGACTTTCCT
ACTTGGCAGTACATCTACGTATTAGTCATCGCTATTACCATGCTG
ATGCGGTTTTGGCAGTACATCAATGGGCGTGGATAGCGGTTTGAC
TCACGGGGATTTCCAAGTCTCCACCCCATTGACGTCAATGGGAGT
TTGTTTTGGCACCAAAATCAACGGGACTTTCCAAAATGTCGTAAC
AACTCCGCCCCATTGACGCAAATGGGCGGTAGGCGTGTACGGTGG
GAGGTCTATATAAGCAGAGCTGGTTTAGTGAACCGTCAGATCAGA
TCTTTGTCGATCCTACCATCCACTCGACACACCCGCCAG
(SEQ ID NO: 45)
LINE-1 plasmid ORF1-E2A-ORF2 GFP
(SEQ ID NO: 46)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGGGAAGCGGACAGTGTACTAATTATGCTCTCT
TGAAATTGGCTGGAGATGTTGAGAGCAACCCTGGACCTATGACCG
GCTCTAACTCACATATCACCATCCTTACACTTAACATTAACGGCC
TCAACTCAGCTATCAAGCGCCATCGGCTGGCCAGCTGGATCAAAT
CACAGGATCCAAGCGTTTGTTGCATCCAAGAGACCCACCTGACCT
GTAGAGATACTCACCGCCTCAAGATCAAGGGATGGCGAAAGATTT
ATCAGGCGAACGGTAAGCAGAAGAAAGCCGGAGTCGCAATTCTGG
TCTCAGACAAGACGGATTTCAAGCCCACCAAAATTAAGCGTGATA
AGGAAGGTCACTATATTATGGTGAAAGGCAGCATACAGCAGGAAG
AACTTACCATATTGAACATCTACGCGCCAAACACCGGCGCACCTC
GCTTTATCAAACAGGTCCTGTCCGATCTGCAGCGAGATCTGGATT
CTCATACGTTGATTATGGGTGATTTCAATACACCATTGAGCACCC
TGGATCGCAGCACCAGGCAAAAGGTAAATAAAGACACGCAAGAGC
TCAATAGCGCACTGCATCAGGCAGATCTCATTGATATTTATCGCA
CTCTTCATCCTAAGAGTACCGAGTACACATTCTTCAGCGCCCCAC
ATCATACATACTCAAAGATCGATCATATCGTCGGCTCAAAGGCTC
TGCTGTCAAAGTGCAAGCGCACAGAGATAATTACAAATTACCTGT
CAGATCATAGCGCGATCAAGCTCGAGCTGAGAATCAAGAACCTGA
CCCAGAGCCGGAGTACCACTTGGAAGCTTAATAACCTGCTGCTCA
ACGATTATTGGGTCCACAATGAGATGAAGGCAGAGATTAAAATGT
TCTTCGAAACAAATGAGAATAAGGATACTACCTATCAAAACCTTT
GGGATGCCTTTAAGGCCGTCTGCAGAGGCAAGTTCATCGCCCTCA
ACGCCTATAAAAGAAAACAAGAGAGATCTAAGATCGATACTCTCA
CCTCTCAGCTGAAGGAGTTGGAGAAACAGGAACAGACCCACTCCA
AGGCGTCAAGACGGCAGGAGATCACAAAGATTCGCGCCGAGTTGA
AAGAGATCGAAACCCAAAAGACTCTTCAGAAAATTAACGAGTCTC
GTAGTTGGTTCTTCGAGCGGATTAATAAGATAGACAGACCTCTGG
CACGACTGATTAAGAAGAAGCGCGAAAAGAACCAGATTGATACCA
TCAAGAACGACAAGGGCGACATCACTACTGACCCGACCGAGATCC
AGACCACTATTCGGGAGTATTATAAGCATTTGTATGCTAACAAGC
TTGAGAACCTGGAAGAGATGGACACTTTTCTGGATACCTATACTC
TGCCACGGCTTAATCAAGAGGAAGTCGAGTCCCTCAACCGCCCAA
TTACAGGAAGCGAGATTGTGGCCATAATTAACTCCCTGCCGACAA
AGAAATCTCCTGGTCCGGACGGGTTTACAGCTGAGTTTTATCAAC
GGTATATGGAAGAGCTTGTACCGTTTCTGCTCAAGCTCTTTCAGT
CTATAGAAAAGGAAGGCATCTTGCCCAATTCCTTCTACGAAGCTT
CTATAATACTTATTCCCAAACCAGGACGCGATACCACAAAGAAGG
AAAACTTCCGGCCCATTAGTCTCATGAATATCGACGCTAAAATAT
TGAACAAGATTCTCGCCAACAGAATCCAACAACATATTAAGAAAT
TGATACATCACGACCAGGTGGGGTTTATACCTGGCATGCAGGGCT
GGTTTAACATCCGGAAGAGTATTAACGTCATTCAACACATTAATA
GAGCTAAGGATAAGAATCATATGATCATCTCTATAGACGCGGAAA
AGGCATTCGATAAGATTCAGCAGCCATTTATGCTCAAGACTCTGA
ACAAACTCGGCATCGACGGAACATATTTTAAGATTATTCGCGCAA
TTTACGATAAGCCGACTGCTAACATTATCCTTAACGGCCAAAAGC
TCGAGGCCTTTCCGCTCAAGACTGGAACCCGCCAAGGCTGTCCCC
TCTCCCCGCTTTTGTTTAATATTGTACTCGAGGTGCTGGCTAGGG
CTATTCGTCAAGAGAAAGAGATTAAAGGGATACAGCTCGGGAAGG
AAGAGGTCAAGCTTTCCTTGTTCGCCGATGATATGATTGTGTACC
TGGAGAATCCTATTGTGTCTGCTCAGAACCTTCTTAAACTTATTT
CTAACTTTAGCAAGGTCAGCGGCTATAAGATTAACGTCCAGAAAT
CTCAGGCCTTTCTGTACACAAATAATCGACAGACCGAATCCCAGA
TAATGGGTGAGCTTCCGTTTGTCATAGCCAGCAAAAGGATAAAGT
ATCTCGGAATCCAGCTGACACGAGACGTTAAAGATTTGTTTAAGG
AAAATTACAAGCCTCTCCTGAAAGAGATTAAGGAAGATACTAATA
AGTGGAAGAATATCCCCTGTTCATGGGTTGGCAGAATCAACATAG
TGAAGATGGCAATACTTCCTAAAGTGATATATCGCTTTAACGCCA
TCCCAATTAAACTGCCTATGACCTTCTTTACGGAGCTCGAGAAAA
CAACCCTTAAATTTATATGGAATCAAAAGAGAGCAAGAATAGCGA
AGTCCATCTTGAGCCAGAAGAATAAGGCCGGTGGGATTACTTTGC
CTGATTTTAAGTTGTATTATAAAGCCACAGTAACTAAGACAGCCT
GGTATTGGTATCAGAATAGAGACATCGACCAGTGGAATCGGACCG
AACCATCAGAGATAATGCCCCACATCTATAATTACCTTATATTCG
ATAAGCCAGAAAAGAATAAACAGTGGGGCAAAGACAGCCTCTTCA
ACAAGTGGTGTTGGGAGAATTGGCTGGCCATATGCCGGAAACTCA
AGCTCGACCCCTTTCTTACACCCTACACTAAAATCAACAGTAGGT
GGATCAAGGACTTGAATGTCAAGCCAAAGACTATAAAGACACTGG
AAGAGAATCTTGGGATCACAATACAAGATATAGGCGTCGGCAAAG
ATTTTATGTCAAAGACGCCCAAGGCCATGGCCACTAAGGATAAGA
TTGATAAGTGGGACCTTATTAAGCTCAAAAGCTTCTGTACTGCCA
AGGAGACCACGATCAGAGTTAATAGGCAGCCCACTACATGGGAAA
AGATTTTCGCCACTTATTCATCAGATAAGGGGTTGATAAGCAGAA
TATATAACGAGCTGAAGCAGATCTACAAGAAGAAAACGAATAATC
CCATCAAGAAGTGGGCAAAAGATATGAACAGGCATTTTAGCAAAG
AGGATATCTACGCCGCGAAGAAGCATATGAAGAAGTGTAGTTCAA
GCTTGGCCATTCGTGAGATGCAGATTAAGACGACCATGCGATACC
ACCTTACCCCAGTGAGGATGGCAATTATCAAGAAATCTGGCAATA
ATAGATGTTGGCGGGGCTGTGGCGAGATTGGCACCCTGCTCCATT
GCTGGTGGGATTGCAAGCTGGTGCAGCCGCTTTGGAAATCAGTCT
GGCGCTTTCTGAGGGACCTCGAGCTTGAGATTCCCTTCGATCCCG
CAATTCCCTTGCTCGGAATCTATCCTAACGAATACAAGAGCTGTT
GTTACAAGGATACGTGTACCCGGATGTTCATCGCGGCCTTGTTTA
CGATAGCTAAGACGTGGAATCAGCCTAAGTGCCCCACAATGATCG
ATTGGATCAAGAAAATGTGGCATATTTATACCATGGAGTATTACG
CAGCAATTAAGAATGACGAATTTATTTCCTTCGTTGGGACCTGGA
TGAAGCTGGAGACTATTATTCTGAGCAAGCTGTCTCAGGAGCAAA
AGACAAAGCATAGAATCTTCTCTCTCATTGGTGGTAACGACTACA
AAGACGATGACGACAAGTAAAGCGCTTCTAGAAGTTGTCTCCTCC
TGCACTGACTGACTGATACAATCGATTTCTGGATCCGCAGGCCTA
ATCAACCTCTGGATTACAAAATTTGTGAAAGATTGACTGGTATTC
TTAACTATGTTGCTCCTTTTACGCTATGTGGATACGCTGCTTTAA
TGCCTTTGTATCATGCTATTGCTTCCCGTATGGCTTTCATTTTCT
CCTCCTTGTATAAATCCTGGTTGCTGTCTCTTTATGAGGAGTTGT
GGCCCGTTGTCAGGCAACGTGGCGTGGTGTGCACTGTGTTTGCTG
ACGCAACCCCCACTGGTTGGGGCATTGCCACCACCTGTCAGCTCC
TTTCCGGGACTTTCGCTTTCCCCCTCCCTATTGCCACGGCGGAAC
TCATCGCCGCCTGCCTTGCCCGCTGCTGGACAGGGGCTCGGCTGT
TGGGCACTGACAATTCCGTGGTGTTGTCGGGGAAGCTGACGTCCT
TTCCATGGCTGCTCGCCTGTGTTGCCACCTGGATTCTGCGCGGGA
CGTCCTTCTGCTACGTCCCTTCGGCCCTCAATCCAGCGGACCTTC
CTTCCCGCGAACAAACGACCCAACACCCGTGCGTTTTATTCTGTC
TTTTTATTGCCGATCCCCTCAGAAGAACTCGTCAAGAAGGCGATA
GAAGGCGATGCGCTGCGAATCGGGAGCGGCGATACCGTAAAGCAC
GAGGAAGCGGTCAGCCCATTCGCCGCCAAGCTCTTCAGCAATATC
ACGGGTAGCCAACGCTATGTCCTGATAGCGGTCGGCCGCTTTACT
TGTACAGCTCGTCCATGCCGAGAGTGATCCCGGCGGCGGTCACGA
ACTCCAGCAGGACCATGTGATCGCGCTTCTCGTTGGGGTCTTTGC
TCAGGGCGGACTGGGTGCTCAGGTAGTGGTTGTCGGGCAGCAGCA
CGGGGCCGTCGCCGATGGGGGTGTTCTGCTGGTAGTGGTCGGCCA
GGTGAGTCCAGGAGATGTTTCAGCACTGTTGCCTTTAGTCTCGAG
GCAACTTAGACAACTGAGTATTGATCTGAGCACAGCAGGGTGTGA
GCTGTTTGAAGATACTGGGGTTGGGGGTGAAGAAACTGCAGAGGA
CTAACTGGGCTGAGACCCAGTGGCAATGTTTTAGGGCCTAAGGAA
TGCCTCTGAAAATCTAGATGGACAACTTTGACTTTGAGAAAAGAG
AGGTGGAAATGAGGAAAATGACTTTTCTTTATTAGATTTCGGTAG
AAAGAACTTTCATCTTTCCCCTATTTTTGTTATTCGTTTTAAAAC
ATCTATCTGGAGGCAGGACAAGTATGGTCATTAAAAAGATGCAGG
CAGAAGGCATATATTGGCTCAGTCAAAGTGGGGAACTTTGGTGGC
CAAACATACATTGCTAAGGCTATTCCTATATCAGCTGGACACATA
TAAAATGCTGCTAATGCTTCATTACAAACTTATATCCTTTAATTC
CAGATGGGGGCAAAGTATGTCCAGGGGTGAGGAACAATTGAAACA
TTTGGGCTGGAGTAGATTTTGAAAGTCAGCTCTGTGTGTGTGTGT
GTGTGTGTGTGTGTGAGAGCGTGTGTTTCTTTTAACGTTTTCAGC
CTACAGCATACAGGGTTCATGGTGGCAAGAAGATAACAAGATTTA
AATTATGGCCAGTGACTAGTGCTGCAAGAAGAACAACTACCTGCA
TTTAATGGGAAAGCAAAATCTCAGGCTTTGAGGGAAGTTAACATA
GGCTTGATTCTGGGTGGAAGCTGGGTGTGTAGTTATCTGGAGGCC
AGGCTGGAGCTCTCAGCTCACTATGGGTTCATCTTTATTGTCTCC
TTTCATCTCAACAGCTGCACGCTGCCGTCCTCGATGTTGTGGCGG
ATCTTGAAGTTCACCTTGATGCCGTTCTTCTGCTTGTCGGCCATG
ATATAGACGTTGTGGCTGTTGTAGTTGTACTCCAGCTTGTGCCCC
AGGATGTTGCCGTCCTCCTTGAAGTCGATGCCCTTCAGCTCGATG
CGGTTCACCAGGGTGTCGCCCTCGAACTTCACCTCGGCGCGGGTC
TTGTAGTTGCCGTCGTCCTTGAAGAAGATGGTGCGCTCCTGGACG
TAGCCTTCGGGCATGGCGGACTTGAAGAAGTCGTGCTGCTTCATG
TGGTCGGGGTAGCGGCTGAAGCACTGCACGCCGTAGGTCAGGGTG
GTCACGAGGGTGGGCCAGGGCACGGGCAGCTTGCCGGTGGTGCAG
ATGAACTTCAGGGTCAGCTTGCCGTAGGTGGCATCGCCCTCGCCC
TCGCCGGACACGCTGAACTTGTGGCCGTTTACGTCGCCGTCCAGC
TCGACCAGGATGGGCACCACCCCGGTGAACAGCTCCTCGCCCTTG
CTCACCATGGTGGCGAATTCGAAGCTTGAGCACGAGATCTGAGTC
CGGTAGGCCTAGCGGATCTGACGGTTCACTAAACCAGCTCTGCTT
ATATAGACCTCCCACCGTACACGCCTACCGCCCATTTGCGTCAAT
GGGGCGGAGTTGTTACGACATTTTGGAAAGTCCCGTTGATTTTGG
TGCCAAAACAAACTCCCATTGACGTCAATGGGGTGGAGACTTGGA
AATCCCCGTGAGTCAAACCGCTATCCACGCCCATTGATGTACTGC
CAAAACCGCATCACCATGGTAATAGCGATGACTAATACGTAGATG
TACTGCCAAGTAGGAAAGTCCCATAAGGTCATGTACTGGGCATAA
TGCCAGGCGGGCCATTTACCGTCATTGACGTCAATAGGGGGCGTA
CTTGGCATATGATACACTTGATGTACTGCCAAGTGGGCAGTTTAC
CGTAAATACTCCACCCATTGACGTCAATGGAAAGTCCCTATTGGC
GTTACTATGGGAACATACGTCATTATTGACGTCAATGGGCGGGGG
TCGTTGGGCGGTCAGCCAGGCGGGCCATTTACCGTAAGTTATGTA
ACGGGCCTGCTGCCGGCTCTGCGGCCTCTTCCGCGTCTTCGCCTT
CGCCCTCAGACGAGTCGGATCTCCCTTTGGGCCGCCTCCCCGCCT
GTCTAGCTTGACTGACTGAGATACAGCGTACCTTCAGCTCACAGA
CATGATAAGATACATTGATGAGTTTGGACAAACCACAACTAGAAT
GCAGTGAAAAAAATGCTTTATTTGTGAAATTTGTGATGCTATTGC
TTTATTTGTAACCATTATAAGCTGCAATAAACAAGTTAACAACAA
CAATTGCATTCATTTTATGTTTCAGGTTCAGGGGGAGGTGTGGGA
GGTTTTTTAAAGCAAGTAAAACCTCTACAAATGTGGTATTGGCCC
ATCTCTATCGGTATCGTAGCATAACCCCTTGGGGCCTCTAAACGG
GTCTTGAGGGGTTTTTTGTGCCCCTCGGGCCGGATTGCTATCTAC
CGGCATTGGCGCAGAAAAAAATGCCTGATGCGACGCTGCGCGTCT
TATACTCCCACATATGCCAGATTCAGCAACGGATACGGCTTCCCC
AACTTGCCCACTTCCATACGTGTCCTCCTTACCAGAAATTTATCC
TTAAGGTCGTCAGCTATCCTGCAGGCGATCTCTCGATTTCGATCA
AGACATTCCTTTAATGGTCTTTTCTGGACACCACTAGGGGTCAGA
AGTAGTTCATCAAACTTTCTTCCCTCCCTAATCTCATTGGTTACC
TTGGGCTATCGAAACTTAATTAAGCGATCTGCATCTCAATTAGTC
AGCAACCATAGTCCCGCCCCTAACTCCGCCCATCCCGCCCCTAAC
TCCGCCCAGTTCCGCCCATTCTCCGCCCCATCGCTGACTAATTTT
TTTTATTTATGCAGAGGCCGAGGCCGCCTCGGCCTCTGAGCTATT
CCAGAAGTAGTGAGGAGGCTTTTTTGGAGGCCTAGGCTTTTGCAA
AGGAGGTAGCCAACATGATTGAACAAGATGGATTGCACGCAGGTT
CTCCCGCCGCTTGGGTGGAGAGGCTATTCGGCTATGACTGGGCAC
AACAGACAATCGGCTGCTCTGATGCCGCCGTGTTCCGGCTGTCAG
CGCAGGGGCGCCCGGTTCTTTTTGTCAAGACCGACCTGTCCGGTG
CCCTGAATGAACTCCAGGACGAGGCAGCGCGGCTATCGTGGCTGG
CCACGACGGGCGTTCCTTGCGCAGCTGTGCTCGACGTTGTCACTG
AAGCGGGAAGGGACTGGCTGCTATTGGGCGAAGTGCCGGGGCAGG
ATCTCCTGTCATCTCACCTTGCTCCTGCCGAGAAAGTATCCATCA
TGGCTGATGCAATGCGGCGGCTGCATACGCTTGATCCGGCTACCT
GCCCATTCGACCACCAAGCGAAACATCGCATCGAGCGAGCACGTA
CTCGGATGGAAGCCGGTCTTGTCGATCAGGATGATCTGGACGAAG
AGCATCAGGGGCTCGCGCCAGCCGAACTGTTCGCCAGGCTCAAGG
CGCGGATGCCCGACGGCGAGGATCTCGTCGTGACCCACGGCGATG
CCTGCTTGCCGAATATCATGGTGGAAAATGGCCGCTTTTCTGGAT
TCATCGACTGTGGCCGGCTGGGTGTGGCGGACCGCTATCAGGACA
TAGCGTTGGCTACCCGTGATATTGCTGAAGAGCTTGGCGGCGAAT
GGGCTGACCGCTTCCTCGTGCTTTACGGTATCGCCGCTCCCGATT
CGCAGCGCATCGCCTTCTATCGCCTTCTTGACGAGTTCTTCTAGT
ATGTAAGCCCTGTGCCTTCTAGTTGCCAGCCATCTGTTGTTTGCC
CCTCCCCCGTGCCTTCCTTGACCCTGGAAGGTGCCACTCCCACTG
TCCTTTCCTAATAAAATGAGGAAATTGCATCGCATTGTCTGAGTA
GGTGTCATTCTATTCTGGGGGGTGGGGTGGGGCAGGACAGCAAGG
GGGAGGATTGGGAAGACAATAGCAGGCATGCTGGGGATGCGGTGG
GCTCTATGGTTAATTAACCAGTCAAGTCAGCTACTTGGCGAGATC
GACTTGTCTGGGTTTCGACTACGCTCAGAATTGCGTCAGTCAAGT
TCGATCTGGTCCTTGCTATTGCACCCGTTCTCCGATTACGAGTTT
CATTTAAATCATGTGAGCAAAAGGCCAGCAAAAGGCCAGGAACCG
TAAAAAGGCCGCGTTGCTGGCGTTTTTCCATAGGCTCCGCCCCCC
TGACGAGCATCACAAAAATCGACGCTCAAGTCAGAGGTGGCGAAA
CCCGACAGGACTATAAAGATACCAGGCGTTTCCCCCTGGAAGCTC
CCTCGTGCGCTCTCCTGTTCCGACCCTGCCGCTTACCGGATACCT
GTCCGCCTTTCTCCCTTCGGGAAGCGTGGCGCTTTCTCATAGCTC
ACGCTGTAGGTATCTCAGTTCGGTGTAGGTCGTTCGCTCCAAGCT
GGGCTGTGTGCACGAACCCCCCGTTCAGCCCGACCGCTGCGCCTT
ATCCGGTAACTATCGTCTTGAGTCCAACCCGGTAAGACACGACTT
ATCGCCACTGGCAGCAGCCACTGGTAACAGGATTAGCAGAGCGAG
GTATGTAGGCGGTGCTACAGAGTTCTTGAAGTGGTGGCCTAACTA
CGGCTACACTAGAAGAACAGTATTTGGTATCTGCGCTCTGCTGAA
GCCAGTTACCTTCGGAAAAAGAGTTGGTAGCTCTTGATCCGGCAA
ACAAACCACCGCTGGTAGCGGTGGTTTTTTTGTTTGCAAGCAGCA
GATTACGCGCAGAAAAAAAGGATCTCAAGAAGATCCTTTGATCTT
TTCTACGGGGTCTGACGCTCAGTGGAACGAAAACTCACGTTAAGG
GATTTTGGTCATGAGATTATCAAAAAGGATCTTCACCTAGATCCT
TTTAAATTAAAAATGAAGTTTTAAATCAATCTAAAGTATATATGA
GTAAACTTGGTCTGACAGTTACCAATGCTTAATCAGTGAGGCACC
TATCTCAGCGATCTGTCTATTTCGTTCATCCATAGTTGCATTTAA
ATTTCCGAACTCTCCAAGGCCCTCGTCGGAAAATCTTCAAACCTT
TCGTCCGATCCATCTTGCAGGCTACCTCTCGAACGAACTATCGCA
AGTCTCTTGGCCGGCCTTGCGCCTTGGCTATTGCTTGGCAGCGCC
TATCGCCAGGTATTACTCCAATCCCGAATATCCGAGATCGGGATC
ACCCGAGAGAAGTTCAACCTACATCCTCAATCCCGATCTATCCGA
GATCCGAGGAATATCGAAATCGGGGCGCGCCTGGTGTACCGAGAA
CGATCCTCTCAGTGCGAGTCTCGACGATCCATATCGTTGCTTGGC
AGTCAGCCAGTCGGAATCCAGCTTGGGACCCAGGAAGTCCAATCG
TCAGATATTGTACTCAAGCCTGGTCACGGCAGCGTACCGATCTGT
TTAAACCTAGATATTGATAGTCTGATCGGTCAACGTATAATCGAG
TCCTAGCTTTTGCAAACATCTATCAAGAGACAGGATCAGCAGGAG
GCTTTCGCATGAGTATTCAACATTTCCGTGTCGCCCTTATTCCCT
TTTTTGCGGCATTTTGCCTTCCTGTTTTTGCTCACCCAGAAACGC
TGGTGAAAGTAAAAGATGCTGAAGATCAGTTGGGTGCGCGAGTGG
GTTACATCGAACTGGATCTCAACAGCGGTAAGATCCTTGAGAGTT
TTCGCCCCGAAGAACGCTTTCCAATGATGAGCACTTTTAAAGTTC
TGCTATGTGGCGCGGTATTATCCCGTATTGACGCCGGGCAAGAGC
AACTCGGTCGCCGCATACACTATTCTCAGAATGACTTGGTTGAGT
ATTCACCAGTCACAGAAAAGCATCTTACGGATGGCATGACAGTAA
GAGAATTATGCAGTGCTGCCATAACCATGAGTGATAACACTGCGG
CCAACTTACTTCTGACAACGATTGGAGGACCGAAGGAGCTAACCG
CTTTTTTGCACAACATGGGGGATCATGTAACTCGCCTTGATCGTT
GGGAACCGGAGCTGAATGAAGCCATACCAAACGACGAGCGTGACA
CCACGATGCCTGTAGCAATGGCAACAACCTTGCGTAAACTATTAA
CTGGCGAACTACTTACTCTAGCTTCCCGGCAACAGTTGATAGACT
GGATGGAGGCGGATAAAGTTGCAGGACCACTTCTGCGCTCGGCCC
TTCCGGCTGGCTGGTTTATTGCTGATAAATCTGGAGCCGGTGAGC
GTGGGTCTCGCGGTATCATTGCAGCACTGGGGCCAGATGGTAAGC
CCTCCCGTATCGTAGTTATCTACACGACGGGGAGTCAGGCAACTA
TGGATGAACGAAATAGACAGATCGCTGAGATAGGTGCCTCACTGA
TTAAGCATTGGTAACCGATTCTAGGTGCATTGGCGCAGAAAAAAA
TGCCTGATGCGACGCTGCGCGTCTTATACTCCCACATATGCCAGA
TTCAGCAACGGATACGGCTTCCCCAACTTGCCCACTTCCATACGT
GTCCTCCTTACCAGAAATTTATCCTTAAGATCGTTTAAACTCGAC
TCTGGCTCTATCGAATCTCCGTCGTTTCGAGCTTACGCGAACAGC
CGTGGCGCTCATTTGCTCGTCGGGCATCGAATCTCGTCAGCTATC
GTCAGCTTACCTTTTTGGCAGCGATCGCGGCTCCCGACATCTTGG
ACCATTAGCTCCACAGGTATCTTCTTCCCTCTAGTGGTCATAACA
GCAGCTTCAGCTACCTCTCAATTCAAAAAACCCCTCAAGACCCGT
TTAGAGGCCCCAAGGGGTTATGCTATCAATCGTTGCGTTACACAC
ACAAAAAACCAACACACATCCATCTTCGATGGATAGCGATTTTAT
TATCTAACTGCTGATCGAGTGTAGCCAGATCTAGTAATCAATTAC
GGGGTCATTAGTTCATAGCCCATATATGGAGTTCCGCGTTACATA
ACTTACGGTAAATGGCCCGCCTGGCTGACCGCCCAACGACCCCCG
CCCATTGACGTCAATAATGACGTATGTTCCCATAGTAACGCCAAT
AGGGACTTTCCATTGACGTCAATGGGTGGAGTATTTACGGTAAAC
TGCCCACTTGGCAGTACATCAAGTGTATCATATGCCAAGTACGCC
CCCTATTGACGTCAATGACGGTAAATGGCCCGCCTGGCATTATGC
CCAGTACATGACCTTATGGGACTTTCCTACTTGGCAGTACATCTA
CGTATTAGTCATCGCTATTACCATGCTGATGCGGTTTTGGCAGTA
CATCAATGGGCGTGGATAGCGGTTTGACTCACGGGGATTTCCAAG
TCTCCACCCCATTGACGTCAATGGGAGTTTGTTTTGGCACCAAAA
TCAACGGGACTTTCCAAAATGTCGTAACAACTCCGCCCCATTGAC
GCAAATGGGCGGTAGGCGTGTACGGTGGGAGGTCTATATAAGCAG
AGCTGGTTTAGTGAACCGTCAGATCAGATCTTTGTCGATCCTACC
ATCCACTCGACACACCCGCCAGCGGCCGC
(SEQ ID NO: 46)
LINE-1 plasmid ORF1-P2A-ORF2 GFP
(SEQ ID NO: 47)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGGGAAGCGGAGCTACTAACTTCAGCCTGCTGA
AGCAGGCTGGAGACGTGGAGGAGAACCCTGGACCTATGACCGGCT
CTAACTCACATATCACCATCCTTACACTTAACATTAACGGCCTCA
ACTCAGCTATCAAGCGCCATCGGCTGGCCAGCTGGATCAAATCAC
AGGATCCAAGCGTTTGTTGCATCCAAGAGACCCACCTGACCTGTA
GAGATACTCACCGCCTCAAGATCAAGGGATGGCGAAAGATTTATC
AGGCGAACGGTAAGCAGAAGAAAGCCGGAGTCGCAATTCTGGTCT
CAGACAAGACGGATTTCAAGCCCACCAAAATTAAGCGTGATAAGG
AAGGTCACTATATTATGGTGAAAGGCAGCATACAGCAGGAAGAAC
TTACCATATTGAACATCTACGCGCCAAACACCGGCGCACCTCGCT
TTATCAAACAGGTCCTGTCCGATCTGCAGCGAGATCTGGATTCTC
ATACGTTGATTATGGGTGATTTCAATACACCATTGAGCACCCTGG
ATCGCAGCACCAGGCAAAAGGTAAATAAAGACACGCAAGAGCTCA
ATAGCGCACTGCATCAGGCAGATCTCATTGATATTTATCGCACTC
TTCATCCTAAGAGTACCGAGTACACATTCTTCAGCGCCCCACATC
ATACATACTCAAAGATCGATCATATCGTCGGCTCAAAGGCTCTGC
TGTCAAAGTGCAAGCGCACAGAGATAATTACAAATTACCTGTCAG
ATCATAGCGCGATCAAGCTCGAGCTGAGAATCAAGAACCTGACCC
AGAGCCGGAGTACCACTTGGAAGCTTAATAACCTGCTGCTCAACG
ATTATTGGGTCCACAATGAGATGAAGGCAGAGATTAAAATGTTCT
TCGAAACAAATGAGAATAAGGATACTACCTATCAAAACCTTTGGG
ATGCCTTTAAGGCCGTCTGCAGAGGCAAGTTCATCGCCCTCAACG
CCTATAAAAGAAAACAAGAGAGATCTAAGATCGATACTCTCACCT
CTCAGCTGAAGGAGTTGGAGAAACAGGAACAGACCCACTCCAAGG
CGTCAAGACGGCAGGAGATCACAAAGATTCGCGCCGAGTTGAAAG
AGATCGAAACCCAAAAGACTCTTCAGAAAATTAACGAGTCTCGTA
GTTGGTTCTTCGAGCGGATTAATAAGATAGACAGACCTCTGGCAC
GACTGATTAAGAAGAAGCGCGAAAAGAACCAGATTGATACCATCA
AGAACGACAAGGGCGACATCACTACTGACCCGACCGAGATCCAGA
CCACTATTCGGGAGTATTATAAGCATTTGTATGCTAACAAGCTTG
AGAACCTGGAAGAGATGGACACTTTTCTGGATACCTATACTCTGC
CACGGCTTAATCAAGAGGAAGTCGAGTCCCTCAACCGCCCAATTA
CAGGAAGCGAGATTGTGGCCATAATTAACTCCCTGCCGACAAAGA
AATCTCCTGGTCCGGACGGGTTTACAGCTGAGTTTTATCAACGGT
ATATGGAAGAGCTTGTACCGTTTCTGCTCAAGCTCTTTCAGTCTA
TAGAAAAGGAAGGCATCTTGCCCAATTCCTTCTACGAAGCTTCTA
TAATACTTATTCCCAAACCAGGACGCGATACCACAAAGAAGGAAA
ACTTCCGGCCCATTAGTCTCATGAATATCGACGCTAAAATATTGA
ACAAGATTCTCGCCAACAGAATCCAACAACATATTAAGAAATTGA
TACATCACGACCAGGTGGGGTTTATACCTGGCATGCAGGGCTGGT
TTAACATCCGGAAGAGTATTAACGTCATTCAACACATTAATAGAG
CTAAGGATAAGAATCATATGATCATCTCTATAGACGCGGAAAAGG
CATTCGATAAGATTCAGCAGCCATTTATGCTCAAGACTCTGAACA
AACTCGGCATCGACGGAACATATTTTAAGATTATTCGCGCAATTT
ACGATAAGCCGACTGCTAACATTATCCTTAACGGCCAAAAGCTCG
AGGCCTTTCCGCTCAAGACTGGAACCCGCCAAGGCTGTCCCCTCT
CCCCGCTTTTGTTTAATATTGTACTCGAGGTGCTGGCTAGGGCTA
TTCGTCAAGAGAAAGAGATTAAAGGGATACAGCTCGGGAAGGAAG
AGGTCAAGCTTTCCTTGTTCGCCGATGATATGATTGTGTACCTGG
AGAATCCTATTGTGTCTGCTCAGAACCTTCTTAAACTTATTTCTA
ACTTTAGCAAGGTCAGCGGCTATAAGATTAACGTCCAGAAATCTC
AGGCCTTTCTGTACACAAATAATCGACAGACCGAATCCCAGATAA
TGGGTGAGCTTCCGTTTGTCATAGCCAGCAAAAGGATAAAGTATC
TCGGAATCCAGCTGACACGAGACGTTAAAGATTTGTTTAAGGAAA
ATTACAAGCCTCTCCTGAAAGAGATTAAGGAAGATACTAATAAGT
GGAAGAATATCCCCTGTTCATGGGTTGGCAGAATCAACATAGTGA
AGATGGCAATACTTCCTAAAGTGATATATCGCTTTAACGCCATCC
CAATTAAACTGCCTATGACCTTCTTTACGGAGCTCGAGAAAACAA
CCCTTAAATTTATATGGAATCAAAAGAGAGCAAGAATAGCGAAGT
CCATCTTGAGCCAGAAGAATAAGGCCGGTGGGATTACTTTGCCTG
ATTTTAAGTTGTATTATAAAGCCACAGTAACTAAGACAGCCTGGT
ATTGGTATCAGAATAGAGACATCGACCAGTGGAATCGGACCGAAC
CATCAGAGATAATGCCCCACATCTATAATTACCTTATATTCGATA
AGCCAGAAAAGAATAAACAGTGGGGCAAAGACAGCCTCTTCAACA
AGTGGTGTTGGGAGAATTGGCTGGCCATATGCCGGAAACTCAAGC
TCGACCCCTTTCTTACACCCTACACTAAAATCAACAGTAGGTGGA
TCAAGGACTTGAATGTCAAGCCAAAGACTATAAAGACACTGGAAG
AGAATCTTGGGATCACAATACAAGATATAGGCGTCGGCAAAGATT
TTATGTCAAAGACGCCCAAGGCCATGGCCACTAAGGATAAGATTG
ATAAGTGGGACCTTATTAAGCTCAAAAGCTTCTGTACTGCCAAGG
AGACCACGATCAGAGTTAATAGGCAGCCCACTACATGGGAAAAGA
TTTTCGCCACTTATTCATCAGATAAGGGGTTGATAAGCAGAATAT
ATAACGAGCTGAAGCAGATCTACAAGAAGAAAACGAATAATCCCA
TCAAGAAGTGGGCAAAAGATATGAACAGGCATTTTAGCAAAGAGG
ATATCTACGCCGCGAAGAAGCATATGAAGAAGTGTAGTTCAAGCT
TGGCCATTCGTGAGATGCAGATTAAGACGACCATGCGATACCACC
TTACCCCAGTGAGGATGGCAATTATCAAGAAATCTGGCAATAATA
GATGTTGGCGGGGCTGTGGCGAGATTGGCACCCTGCTCCATTGCT
GGTGGGATTGCAAGCTGGTGCAGCCGCTTTGGAAATCAGTCTGGC
GCTTTCTGAGGGACCTCGAGCTTGAGATTCCCTTCGATCCCGCAA
TTCCCTTGCTCGGAATCTATCCTAACGAATACAAGAGCTGTTGTT
ACAAGGATACGTGTACCCGGATGTTCATCGCGGCCTTGTTTACGA
TAGCTAAGACGTGGAATCAGCCTAAGTGCCCCACAATGATCGATT
GGATCAAGAAAATGTGGCATATTTATACCATGGAGTATTACGCAG
CAATTAAGAATGACGAATTTATTTCCTTCGTTGGGACCTGGATGA
AGCTGGAGACTATTATTCTGAGCAAGCTGTCTCAGGAGCAAAAGA
CAAAGCATAGAATCTTCTCTCTCATTGGTGGTAACGACTACAAAG
ACGATGACGACAAGTAAAGCGCTTCTAGAAGTTGTCTCCTCCTGC
ACTGACTGACTGATACAATCGATTTCTGGATCCGCAGGCCTAATC
AACCTCTGGATTACAAAATTTGTGAAAGATTGACTGGTATTCTTA
ACTATGTTGCTCCTTTTACGCTATGTGGATACGCTGCTTTAATGC
CTTTGTATCATGCTATTGCTTCCCGTATGGCTTTCATTTTCTCCT
CCTTGTATAAATCCTGGTTGCTGTCTCTTTATGAGGAGTTGTGGC
CCGTTGTCAGGCAACGTGGCGTGGTGTGCACTGTGTTTGCTGACG
CAACCCCCACTGGTTGGGGCATTGCCACCACCTGTCAGCTCCTTT
CCGGGACTTTCGCTTTCCCCCTCCCTATTGCCACGGCGGAACTCA
TCGCCGCCTGCCTTGCCCGCTGCTGGACAGGGGCTCGGCTGTTGG
GCACTGACAATTCCGTGGTGTTGTCGGGGAAGCTGACGTCCTTTC
CATGGCTGCTCGCCTGTGTTGCCACCTGGATTCTGCGCGGGACGT
CCTTCTGCTACGTCCCTTCGGCCCTCAATCCAGCGGACCTTCCTT
CCCGCGAACAAACGACCCAACACCCGTGCGTTTTATTCTGTCTTT
TTATTGCCGATCCCCTCAGAAGAACTCGTCAAGAAGGCGATAGAA
GGCGATGCGCTGCGAATCGGGAGCGGCGATACCGTAAAGCACGAG
GAAGCGGTCAGCCCATTCGCCGCCAAGCTCTTCAGCAATATCACG
GGTAGCCAACGCTATGTCCTGATAGCGGTCGGCCGCTTTACTTGT
ACAGCTCGTCCATGCCGAGAGTGATCCCGGCGGCGGTCACGAACT
CCAGCAGGACCATGTGATCGCGCTTCTCGTTGGGGTCTTTGCTCA
GGGCGGACTGGGTGCTCAGGTAGTGGTTGTCGGGCAGCAGCACGG
GGCCGTCGCCGATGGGGGTGTTCTGCTGGTAGTGGTCGGCCAGGT
GAGTCCAGGAGATGTTTCAGCACTGTTGCCTTTAGTCTCGAGGCA
ACTTAGACAACTGAGTATTGATCTGAGCACAGCAGGGTGTGAGCT
GTTTGAAGATACTGGGGTTGGGGGTGAAGAAACTGCAGAGGACTA
ACTGGGCTGAGACCCAGTGGCAATGTTTTAGGGCCTAAGGAATGC
CTCTGAAAATCTAGATGGACAACTTTGACTTTGAGAAAAGAGAGG
TGGAAATGAGGAAAATGACTTTTCTTTATTAGATTTCGGTAGAAA
GAACTTTCATCTTTCCCCTATTTTTGTTATTCGTTTTAAAACATC
TATCTGGAGGCAGGACAAGTATGGTCATTAAAAAGATGCAGGCAG
AAGGCATATATTGGCTCAGTCAAAGTGGGGAACTTTGGTGGCCAA
ACATACATTGCTAAGGCTATTCCTATATCAGCTGGACACATATAA
AATGCTGCTAATGCTTCATTACAAACTTATATCCTTTAATTCCAG
ATGGGGGCAAAGTATGTCCAGGGGTGAGGAACAATTGAAACATTT
GGGCTGGAGTAGATTTTGAAAGTCAGCTCTGTGTGTGTGTGTGTG
TGTGTGTGTGTGAGAGCGTGTGTTTCTTTTAACGTTTTCAGCCTA
CAGCATACAGGGTTCATGGTGGCAAGAAGATAACAAGATTTAAAT
TATGGCCAGTGACTAGTGCTGCAAGAAGAACAACTACCTGCATTT
AATGGGAAAGCAAAATCTCAGGCTTTGAGGGAAGTTAACATAGGC
TTGATTCTGGGTGGAAGCTGGGTGTGTAGTTATCTGGAGGCCAGG
CTGGAGCTCTCAGCTCACTATGGGTTCATCTTTATTGTCTCCTTT
CATCTCAACAGCTGCACGCTGCCGTCCTCGATGTTGTGGCGGATC
TTGAAGTTCACCTTGATGCCGTTCTTCTGCTTGTCGGCCATGATA
TAGACGTTGTGGCTGTTGTAGTTGTACTCCAGCTTGTGCCCCAGG
ATGTTGCCGTCCTCCTTGAAGTCGATGCCCTTCAGCTCGATGCGG
TTCACCAGGGTGTCGCCCTCGAACTTCACCTCGGCGCGGGTCTTG
TAGTTGCCGTCGTCCTTGAAGAAGATGGTGCGCTCCTGGACGTAG
CCTTCGGGCATGGCGGACTTGAAGAAGTCGTGCTGCTTCATGTGG
TCGGGGTAGCGGCTGAAGCACTGCACGCCGTAGGTCAGGGTGGTC
ACGAGGGTGGGCCAGGGCACGGGCAGCTTGCCGGTGGTGCAGATG
AACTTCAGGGTCAGCTTGCCGTAGGTGGCATCGCCCTCGCCCTCG
CCGGACACGCTGAACTTGTGGCCGTTTACGTCGCCGTCCAGCTCG
ACCAGGATGGGCACCACCCCGGTGAACAGCTCCTCGCCCTTGCTC
ACCATGGTGGCGAATTCGAAGCTTGAGCACGAGATCTGAGTCCGG
TAGGCCTAGCGGATCTGACGGTTCACTAAACCAGCTCTGCTTATA
TAGACCTCCCACCGTACACGCCTACCGCCCATTTGCGTCAATGGG
GCGGAGTTGTTACGACATTTTGGAAAGTCCCGTTGATTTTGGTGC
CAAAACAAACTCCCATTGACGTCAATGGGGTGGAGACTTGGAAAT
CCCCGTGAGTCAAACCGCTATCCACGCCCATTGATGTACTGCCAA
AACCGCATCACCATGGTAATAGCGATGACTAATACGTAGATGTAC
TGCCAAGTAGGAAAGTCCCATAAGGTCATGTACTGGGCATAATGC
CAGGCGGGCCATTTACCGTCATTGACGTCAATAGGGGGCGTACTT
GGCATATGATACACTTGATGTACTGCCAAGTGGGCAGTTTACCGT
AAATACTCCACCCATTGACGTCAATGGAAAGTCCCTATTGGCGTT
ACTATGGGAACATACGTCATTATTGACGTCAATGGGCGGGGGTCG
TTGGGCGGTCAGCCAGGCGGGCCATTTACCGTAAGTTATGTAACG
GGCCTGCTGCCGGCTCTGCGGCCTCTTCCGCGTCTTCGCCTTCGC
CCTCAGACGAGTCGGATCTCCCTTTGGGCCGCCTCCCCGCCTGTC
TAGCTTGACTGACTGAGATACAGCGTACCTTCAGCTCACAGACAT
GATAAGATACATTGATGAGTTTGGACAAACCACAACTAGAATGCA
GTGAAAAAAATGCTTTATTTGTGAAATTTGTGATGCTATTGCTTT
ATTTGTAACCATTATAAGCTGCAATAAACAAGTTAACAACAACAA
TTGCATTCATTTTATGTTTCAGGTTCAGGGGGAGGTGTGGGAGGT
TTTTTAAAGCAAGTAAAACCTCTACAAATGTGGTATTGGCCCATC
TCTATCGGTATCGTAGCATAACCCCTTGGGGCCTCTAAACGGGTC
TTGAGGGGTTTTTTGTGCCCCTCGGGCCGGATTGCTATCTACCGG
CATTGGCGCAGAAAAAAATGCCTGATGCGACGCTGCGCGTCTTAT
ACTCCCACATATGCCAGATTCAGCAACGGATACGGCTTCCCCAAC
TTGCCCACTTCCATACGTGTCCTCCTTACCAGAAATTTATCCTTA
AGGTCGTCAGCTATCCTGCAGGCGATCTCTCGATTTCGATCAAGA
CATTCCTTTAATGGTCTTTTCTGGACACCACTAGGGGTCAGAAGT
AGTTCATCAAACTTTCTTCCCTCCCTAATCTCATTGGTTACCTTG
GGCTATCGAAACTTAATTAAGCGATCTGCATCTCAATTAGTCAGC
AACCATAGTCCCGCCCCTAACTCCGCCCATCCCGCCCCTAACTCC
GCCCAGTTCCGCCCATTCTCCGCCCCATCGCTGACTAATTTTTTT
TATTTATGCAGAGGCCGAGGCCGCCTCGGCCTCTGAGCTATTCCA
GAAGTAGTGAGGAGGCTTTTTTGGAGGCCTAGGCTTTTGCAAAGG
AGGTAGCCAACATGATTGAACAAGATGGATTGCACGCAGGTTCTC
CCGCCGCTTGGGTGGAGAGGCTATTCGGCTATGACTGGGCACAAC
AGACAATCGGCTGCTCTGATGCCGCCGTGTTCCGGCTGTCAGCGC
AGGGGCGCCCGGTTCTTTTTGTCAAGACCGACCTGTCCGGTGCCC
TGAATGAACTCCAGGACGAGGCAGCGCGGCTATCGTGGCTGGCCA
CGACGGGCGTTCCTTGCGCAGCTGTGCTCGACGTTGTCACTGAAG
CGGGAAGGGACTGGCTGCTATTGGGCGAAGTGCCGGGGCAGGATC
TCCTGTCATCTCACCTTGCTCCTGCCGAGAAAGTATCCATCATGG
CTGATGCAATGCGGCGGCTGCATACGCTTGATCCGGCTACCTGCC
CATTCGACCACCAAGCGAAACATCGCATCGAGCGAGCACGTACTC
GGATGGAAGCCGGTCTTGTCGATCAGGATGATCTGGACGAAGAGC
ATCAGGGGCTCGCGCCAGCCGAACTGTTCGCCAGGCTCAAGGCGC
GGATGCCCGACGGCGAGGATCTCGTCGTGACCCACGGCGATGCCT
GCTTGCCGAATATCATGGTGGAAAATGGCCGCTTTTCTGGATTCA
TCGACTGTGGCCGGCTGGGTGTGGCGGACCGCTATCAGGACATAG
CGTTGGCTACCCGTGATATTGCTGAAGAGCTTGGCGGCGAATGGG
CTGACCGCTTCCTCGTGCTTTACGGTATCGCCGCTCCCGATTCGC
AGCGCATCGCCTTCTATCGCCTTCTTGACGAGTTCTTCTAGTATG
TAAGCCCTGTGCCTTCTAGTTGCCAGCCATCTGTTGTTTGCCCCT
CCCCCGTGCCTTCCTTGACCCTGGAAGGTGCCACTCCCACTGTCC
TTTCCTAATAAAATGAGGAAATTGCATCGCATTGTCTGAGTAGGT
GTCATTCTATTCTGGGGGGTGGGGTGGGGCAGGACAGCAAGGGGG
AGGATTGGGAAGACAATAGCAGGCATGCTGGGGATGCGGTGGGCT
CTATGGTTAATTAACCAGTCAAGTCAGCTACTTGGCGAGATCGAC
TTGTCTGGGTTTCGACTACGCTCAGAATTGCGTCAGTCAAGTTCG
ATCTGGTCCTTGCTATTGCACCCGTTCTCCGATTACGAGTTTCAT
TTAAATCATGTGAGCAAAAGGCCAGCAAAAGGCCAGGAACCGTAA
AAAGGCCGCGTTGCTGGCGTTTTTCCATAGGCTCCGCCCCCCTGA
CGAGCATCACAAAAATCGACGCTCAAGTCAGAGGTGGCGAAACCC
GACAGGACTATAAAGATACCAGGCGTTTCCCCCTGGAAGCTCCCT
CGTGCGCTCTCCTGTTCCGACCCTGCCGCTTACCGGATACCTGTC
CGCCTTTCTCCCTTCGGGAAGCGTGGCGCTTTCTCATAGCTCACG
CTGTAGGTATCTCAGTTCGGTGTAGGTCGTTCGCTCCAAGCTGGG
CTGTGTGCACGAACCCCCCGTTCAGCCCGACCGCTGCGCCTTATC
CGGTAACTATCGTCTTGAGTCCAACCCGGTAAGACACGACTTATC
GCCACTGGCAGCAGCCACTGGTAACAGGATTAGCAGAGCGAGGTA
TGTAGGCGGTGCTACAGAGTTCTTGAAGTGGTGGCCTAACTACGG
CTACACTAGAAGAACAGTATTTGGTATCTGCGCTCTGCTGAAGCC
AGTTACCTTCGGAAAAAGAGTTGGTAGCTCTTGATCCGGCAAACA
AACCACCGCTGGTAGCGGTGGTTTTTTTGTTTGCAAGCAGCAGAT
TACGCGCAGAAAAAAAGGATCTCAAGAAGATCCTTTGATCTTTTC
TACGGGGTCTGACGCTCAGTGGAACGAAAACTCACGTTAAGGGAT
TTTGGTCATGAGATTATCAAAAAGGATCTTCACCTAGATCCTTTT
AAATTAAAAATGAAGTTTTAAATCAATCTAAAGTATATATGAGTA
AACTTGGTCTGACAGTTACCAATGCTTAATCAGTGAGGCACCTAT
CTCAGCGATCTGTCTATTTCGTTCATCCATAGTTGCATTTAAATT
TCCGAACTCTCCAAGGCCCTCGTCGGAAAATCTTCAAACCTTTCG
TCCGATCCATCTTGCAGGCTACCTCTCGAACGAACTATCGCAAGT
CTCTTGGCCGGCCTTGCGCCTTGGCTATTGCTTGGCAGCGCCTAT
CGCCAGGTATTACTCCAATCCCGAATATCCGAGATCGGGATCACC
CGAGAGAAGTTCAACCTACATCCTCAATCCCGATCTATCCGAGAT
CCGAGGAATATCGAAATCGGGGCGCGCCTGGTGTACCGAGAACGA
TCCTCTCAGTGCGAGTCTCGACGATCCATATCGTTGCTTGGCAGT
CAGCCAGTCGGAATCCAGCTTGGGACCCAGGAAGTCCAATCGTCA
GATATTGTACTCAAGCCTGGTCACGGCAGCGTACCGATCTGTTTA
AACCTAGATATTGATAGTCTGATCGGTCAACGTATAATCGAGTCC
TAGCTTTTGCAAACATCTATCAAGAGACAGGATCAGCAGGAGGCT
TTCGCATGAGTATTCAACATTTCCGTGTCGCCCTTATTCCCTTTT
TTGCGGCATTTTGCCTTCCTGTTTTTGCTCACCCAGAAACGCTGG
TGAAAGTAAAAGATGCTGAAGATCAGTTGGGTGCGCGAGTGGGTT
ACATCGAACTGGATCTCAACAGCGGTAAGATCCTTGAGAGTTTTC
GCCCCGAAGAACGCTTTCCAATGATGAGCACTTTTAAAGTTCTGC
TATGTGGCGCGGTATTATCCCGTATTGACGCCGGGCAAGAGCAAC
TCGGTCGCCGCATACACTATTCTCAGAATGACTTGGTTGAGTATT
CACCAGTCACAGAAAAGCATCTTACGGATGGCATGACAGTAAGAG
AATTATGCAGTGCTGCCATAACCATGAGTGATAACACTGCGGCCA
ACTTACTTCTGACAACGATTGGAGGACCGAAGGAGCTAACCGCTT
TTTTGCACAACATGGGGGATCATGTAACTCGCCTTGATCGTTGGG
AACCGGAGCTGAATGAAGCCATACCAAACGACGAGCGTGACACCA
CGATGCCTGTAGCAATGGCAACAACCTTGCGTAAACTATTAACTG
GCGAACTACTTACTCTAGCTTCCCGGCAACAGTTGATAGACTGGA
TGGAGGCGGATAAAGTTGCAGGACCACTTCTGCGCTCGGCCCTTC
CGGCTGGCTGGTTTATTGCTGATAAATCTGGAGCCGGTGAGCGTG
GGTCTCGCGGTATCATTGCAGCACTGGGGCCAGATGGTAAGCCCT
CCCGTATCGTAGTTATCTACACGACGGGGAGTCAGGCAACTATGG
ATGAACGAAATAGACAGATCGCTGAGATAGGTGCCTCACTGATTA
AGCATTGGTAACCGATTCTAGGTGCATTGGCGCAGAAAAAAATGC
CTGATGCGACGCTGCGCGTCTTATACTCCCACATATGCCAGATTC
AGCAACGGATACGGCTTCCCCAACTTGCCCACTTCCATACGTGTC
CTCCTTACCAGAAATTTATCCTTAAGATCGTTTAAACTCGACTCT
GGCTCTATCGAATCTCCGTCGTTTCGAGCTTACGCGAACAGCCGT
GGCGCTCATTTGCTCGTCGGGCATCGAATCTCGTCAGCTATCGTC
AGCTTACCTTTTTGGCAGCGATCGCGGCTCCCGACATCTTGGACC
ATTAGCTCCACAGGTATCTTCTTCCCTCTAGTGGTCATAACAGCA
GCTTCAGCTACCTCTCAATTCAAAAAACCCCTCAAGACCCGTTTA
GAGGCCCCAAGGGGTTATGCTATCAATCGTTGCGTTACACACACA
AAAAACCAACACACATCCATCTTCGATGGATAGCGATTTTATTAT
CTAACTGCTGATCGAGTGTAGCCAGATCTAGTAATCAATTACGGG
GTCATTAGTTCATAGCCCATATATGGAGTTCCGCGTTACATAACT
TACGGTAAATGGCCCGCCTGGCTGACCGCCCAACGACCCCCGCCC
ATTGACGTCAATAATGACGTATGTTCCCATAGTAACGCCAATAGG
GACTTTCCATTGACGTCAATGGGTGGAGTATTTACGGTAAACTGC
CCACTTGGCAGTACATCAAGTGTATCATATGCCAAGTACGCCCCC
TATTGACGTCAATGACGGTAAATGGCCCGCCTGGCATTATGCCCA
GTACATGACCTTATGGGACTTTCCTACTTGGCAGTACATCTACGT
ATTAGTCATCGCTATTACCATGCTGATGCGGTTTTGGCAGTACAT
CAATGGGCGTGGATAGCGGTTTGACTCACGGGGATTTCCAAGTCT
CCACCCCATTGACGTCAATGGGAGTTTGTTTTGGCACCAAAATCA
ACGGGACTTTCCAAAATGTCGTAACAACTCCGCCCCATTGACGCA
AATGGGCGGTAGGCGTGTACGGTGGGAGGTCTATATAAGCAGAGC
TGGTTTAGTGAACCGTCAGATCAGATCTTTGTCGATCCTACCATC
CACTCGACACACCCGCCAGCGGCCGC
(SEQ ID NO: 47)
LINE-1 plasmid ORF1-T2A ORF2 GFP
(SEQ ID NO: 48)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGGGAAGCGGAGAGGGCAGAGGAAGTCTGCTAA
CATGCGGTGACGTCGAGGAGAATCCTGGACCTATGACCGGCTCTA
ACTCACATATCACCATCCTTACACTTAACATTAACGGCCTCAACT
CAGCTATCAAGCGCCATCGGCTGGCCAGCTGGATCAAATCACAGG
ATCCAAGCGTTTGTTGCATCCAAGAGACCCACCTGACCTGTAGAG
ATACTCACCGCCTCAAGATCAAGGGATGGCGAAAGATTTATCAGG
CGAACGGTAAGCAGAAGAAAGCCGGAGTCGCAATTCTGGTCTCAG
ACAAGACGGATTTCAAGCCCACCAAAATTAAGCGTGATAAGGAAG
GTCACTATATTATGGTGAAAGGCAGCATACAGCAGGAAGAACTTA
CCATATTGAACATCTACGCGCCAAACACCGGCGCACCTCGCTTTA
TCAAACAGGTCCTGTCCGATCTGCAGCGAGATCTGGATTCTCATA
CGTTGATTATGGGTGATTTCAATACACCATTGAGCACCCTGGATC
GCAGCACCAGGCAAAAGGTAAATAAAGACACGCAAGAGCTCAATA
GCGCACTGCATCAGGCAGATCTCATTGATATTTATCGCACTCTTC
ATCCTAAGAGTACCGAGTACACATTCTTCAGCGCCCCACATCATA
CATACTCAAAGATCGATCATATCGTCGGCTCAAAGGCTCTGCTGT
CAAAGTGCAAGCGCACAGAGATAATTACAAATTACCTGTCAGATC
ATAGCGCGATCAAGCTCGAGCTGAGAATCAAGAACCTGACCCAGA
GCCGGAGTACCACTTGGAAGCTTAATAACCTGCTGCTCAACGATT
ATTGGGTCCACAATGAGATGAAGGCAGAGATTAAAATGTTCTTCG
AAACAAATGAGAATAAGGATACTACCTATCAAAACCTTTGGGATG
CCTTTAAGGCCGTCTGCAGAGGCAAGTTCATCGCCCTCAACGCCT
ATAAAAGAAAACAAGAGAGATCTAAGATCGATACTCTCACCTCTC
AGCTGAAGGAGTTGGAGAAACAGGAACAGACCCACTCCAAGGCGT
CAAGACGGCAGGAGATCACAAAGATTCGCGCCGAGTTGAAAGAGA
TCGAAACCCAAAAGACTCTTCAGAAAATTAACGAGTCTCGTAGTT
GGTTCTTCGAGCGGATTAATAAGATAGACAGACCTCTGGCACGAC
TGATTAAGAAGAAGCGCGAAAAGAACCAGATTGATACCATCAAGA
ACGACAAGGGCGACATCACTACTGACCCGACCGAGATCCAGACCA
CTATTCGGGAGTATTATAAGCATTTGTATGCTAACAAGCTTGAGA
ACCTGGAAGAGATGGACACTTTTCTGGATACCTATACTCTGCCAC
GGCTTAATCAAGAGGAAGTCGAGTCCCTCAACCGCCCAATTACAG
GAAGCGAGATTGTGGCCATAATTAACTCCCTGCCGACAAAGAAAT
CTCCTGGTCCGGACGGGTTTACAGCTGAGTTTTATCAACGGTATA
TGGAAGAGCTTGTACCGTTTCTGCTCAAGCTCTTTCAGTCTATAG
AAAAGGAAGGCATCTTGCCCAATTCCTTCTACGAAGCTTCTATAA
TACTTATTCCCAAACCAGGACGCGATACCACAAAGAAGGAAAACT
TCCGGCCCATTAGTCTCATGAATATCGACGCTAAAATATTGAACA
AGATTCTCGCCAACAGAATCCAACAACATATTAAGAAATTGATAC
ATCACGACCAGGTGGGGTTTATACCTGGCATGCAGGGCTGGTTTA
ACATCCGGAAGAGTATTAACGTCATTCAACACATTAATAGAGCTA
AGGATAAGAATCATATGATCATCTCTATAGACGCGGAAAAGGCAT
TCGATAAGATTCAGCAGCCATTTATGCTCAAGACTCTGAACAAAC
TCGGCATCGACGGAACATATTTTAAGATTATTCGCGCAATTTACG
ATAAGCCGACTGCTAACATTATCCTTAACGGCCAAAAGCTCGAGG
CCTTTCCGCTCAAGACTGGAACCCGCCAAGGCTGTCCCCTCTCCC
CGCTTTTGTTTAATATTGTACTCGAGGTGCTGGCTAGGGCTATTC
GTCAAGAGAAAGAGATTAAAGGGATACAGCTCGGGAAGGAAGAGG
TCAAGCTTTCCTTGTTCGCCGATGATATGATTGTGTACCTGGAGA
ATCCTATTGTGTCTGCTCAGAACCTTCTTAAACTTATTTCTAACT
TTAGCAAGGTCAGCGGCTATAAGATTAACGTCCAGAAATCTCAGG
CCTTTCTGTACACAAATAATCGACAGACCGAATCCCAGATAATGG
GTGAGCTTCCGTTTGTCATAGCCAGCAAAAGGATAAAGTATCTCG
GAATCCAGCTGACACGAGACGTTAAAGATTTGTTTAAGGAAAATT
ACAAGCCTCTCCTGAAAGAGATTAAGGAAGATACTAATAAGTGGA
AGAATATCCCCTGTTCATGGGTTGGCAGAATCAACATAGTGAAGA
TGGCAATACTTCCTAAAGTGATATATCGCTTTAACGCCATCCCAA
TTAAACTGCCTATGACCTTCTTTACGGAGCTCGAGAAAACAACCC
TTAAATTTATATGGAATCAAAAGAGAGCAAGAATAGCGAAGTCCA
TCTTGAGCCAGAAGAATAAGGCCGGTGGGATTACTTTGCCTGATT
TTAAGTTGTATTATAAAGCCACAGTAACTAAGACAGCCTGGTATT
GGTATCAGAATAGAGACATCGACCAGTGGAATCGGACCGAACCAT
CAGAGATAATGCCCCACATCTATAATTACCTTATATTCGATAAGC
CAGAAAAGAATAAACAGTGGGGCAAAGACAGCCTCTTCAACAAGT
GGTGTTGGGAGAATTGGCTGGCCATATGCCGGAAACTCAAGCTCG
ACCCCTTTCTTACACCCTACACTAAAATCAACAGTAGGTGGATCA
AGGACTTGAATGTCAAGCCAAAGACTATAAAGACACTGGAAGAGA
ATCTTGGGATCACAATACAAGATATAGGCGTCGGCAAAGATTTTA
TGTCAAAGACGCCCAAGGCCATGGCCACTAAGGATAAGATTGATA
AGTGGGACCTTATTAAGCTCAAAAGCTTCTGTACTGCCAAGGAGA
CCACGATCAGAGTTAATAGGCAGCCCACTACATGGGAAAAGATTT
TCGCCACTTATTCATCAGATAAGGGGTTGATAAGCAGAATATATA
ACGAGCTGAAGCAGATCTACAAGAAGAAAACGAATAATCCCATCA
AGAAGTGGGCAAAAGATATGAACAGGCATTTTAGCAAAGAGGATA
TCTACGCCGCGAAGAAGCATATGAAGAAGTGTAGTTCAAGCTTGG
CCATTCGTGAGATGCAGATTAAGACGACCATGCGATACCACCTTA
CCCCAGTGAGGATGGCAATTATCAAGAAATCTGGCAATAATAGAT
GTTGGCGGGGCTGTGGCGAGATTGGCACCCTGCTCCATTGCTGGT
GGGATTGCAAGCTGGTGCAGCCGCTTTGGAAATCAGTCTGGCGCT
TTCTGAGGGACCTCGAGCTTGAGATTCCCTTCGATCCCGCAATTC
CCTTGCTCGGAATCTATCCTAACGAATACAAGAGCTGTTGTTACA
AGGATACGTGTACCCGGATGTTCATCGCGGCCTTGTTTACGATAG
CTAAGACGTGGAATCAGCCTAAGTGCCCCACAATGATCGATTGGA
TCAAGAAAATGTGGCATATTTATACCATGGAGTATTACGCAGCAA
TTAAGAATGACGAATTTATTTCCTTCGTTGGGACCTGGATGAAGC
TGGAGACTATTATTCTGAGCAAGCTGTCTCAGGAGCAAAAGACAA
AGCATAGAATCTTCTCTCTCATTGGTGGTAACGACTACAAAGACG
ATGACGACAAGTAAAGCGCTTCTAGAAGTTGTCTCCTCCTGCACT
GACTGACTGATACAATCGATTTCTGGATCCGCAGGCCTAATCAAC
CTCTGGATTACAAAATTTGTGAAAGATTGACTGGTATTCTTAACT
ATGTTGCTCCTTTTACGCTATGTGGATACGCTGCTTTAATGCCTT
TGTATCATGCTATTGCTTCCCGTATGGCTTTCATTTTCTCCTCCT
TGTATAAATCCTGGTTGCTGTCTCTTTATGAGGAGTTGTGGCCCG
TTGTCAGGCAACGTGGCGTGGTGTGCACTGTGTTTGCTGACGCAA
CCCCCACTGGTTGGGGCATTGCCACCACCTGTCAGCTCCTTTCCG
GGACTTTCGCTTTCCCCCTCCCTATTGCCACGGCGGAACTCATCG
CCGCCTGCCTTGCCCGCTGCTGGACAGGGGCTCGGCTGTTGGGCA
CTGACAATTCCGTGGTGTTGTCGGGGAAGCTGACGTCCTTTCCAT
GGCTGCTCGCCTGTGTTGCCACCTGGATTCTGCGCGGGACGTCCT
TCTGCTACGTCCCTTCGGCCCTCAATCCAGCGGACCTTCCTTCCC
GCGAACAAACGACCCAACACCCGTGCGTTTTATTCTGTCTTTTTA
TTGCCGATCCCCTCAGAAGAACTCGTCAAGAAGGCGATAGAAGGC
GATGCGCTGCGAATCGGGAGCGGCGATACCGTAAAGCACGAGGAA
GCGGTCAGCCCATTCGCCGCCAAGCTCTTCAGCAATATCACGGGT
AGCCAACGCTATGTCCTGATAGCGGTCGGCCGCTTTACTTGTACA
GCTCGTCCATGCCGAGAGTGATCCCGGCGGCGGTCACGAACTCCA
GCAGGACCATGTGATCGCGCTTCTCGTTGGGGTCTTTGCTCAGGG
CGGACTGGGTGCTCAGGTAGTGGTTGTCGGGCAGCAGCACGGGGC
CGTCGCCGATGGGGGTGTTCTGCTGGTAGTGGTCGGCCAGGTGAG
TCCAGGAGATGTTTCAGCACTGTTGCCTTTAGTCTCGAGGCAACT
TAGACAACTGAGTATTGATCTGAGCACAGCAGGGTGTGAGCTGTT
TGAAGATACTGGGGTTGGGGGTGAAGAAACTGCAGAGGACTAACT
GGGCTGAGACCCAGTGGCAATGTTTTAGGGCCTAAGGAATGCCTC
TGAAAATCTAGATGGACAACTTTGACTTTGAGAAAAGAGAGGTGG
AAATGAGGAAAATGACTTTTCTTTATTAGATTTCGGTAGAAAGAA
CTTTCATCTTTCCCCTATTTTTGTTATTCGTTTTAAAACATCTAT
CTGGAGGCAGGACAAGTATGGTCATTAAAAAGATGCAGGCAGAAG
GCATATATTGGCTCAGTCAAAGTGGGGAACTTTGGTGGCCAAACA
TACATTGCTAAGGCTATTCCTATATCAGCTGGACACATATAAAAT
GCTGCTAATGCTTCATTACAAACTTATATCCTTTAATTCCAGATG
GGGGCAAAGTATGTCCAGGGGTGAGGAACAATTGAAACATTTGGG
CTGGAGTAGATTTTGAAAGTCAGCTCTGTGTGTGTGTGTGTGTGT
GTGTGTGTGAGAGCGTGTGTTTCTTTTAACGTTTTCAGCCTACAG
CATACAGGGTTCATGGTGGCAAGAAGATAACAAGATTTAAATTAT
GGCCAGTGACTAGTGCTGCAAGAAGAACAACTACCTGCATTTAAT
GGGAAAGCAAAATCTCAGGCTTTGAGGGAAGTTAACATAGGCTTG
ATTCTGGGTGGAAGCTGGGTGTGTAGTTATCTGGAGGCCAGGCTG
GAGCTCTCAGCTCACTATGGGTTCATCTTTATTGTCTCCTTTCAT
CTCAACAGCTGCACGCTGCCGTCCTCGATGTTGTGGCGGATCTTG
AAGTTCACCTTGATGCCGTTCTTCTGCTTGTCGGCCATGATATAG
ACGTTGTGGCTGTTGTAGTTGTACTCCAGCTTGTGCCCCAGGATG
TTGCCGTCCTCCTTGAAGTCGATGCCCTTCAGCTCGATGCGGTTC
ACCAGGGTGTCGCCCTCGAACTTCACCTCGGCGCGGGTCTTGTAG
TTGCCGTCGTCCTTGAAGAAGATGGTGCGCTCCTGGACGTAGCCT
TCGGGCATGGCGGACTTGAAGAAGTCGTGCTGCTTCATGTGGTCG
GGGTAGCGGCTGAAGCACTGCACGCCGTAGGTCAGGGTGGTCACG
AGGGTGGGCCAGGGCACGGGCAGCTTGCCGGTGGTGCAGATGAAC
TTCAGGGTCAGCTTGCCGTAGGTGGCATCGCCCTCGCCCTCGCCG
GACACGCTGAACTTGTGGCCGTTTACGTCGCCGTCCAGCTCGACC
AGGATGGGCACCACCCCGGTGAACAGCTCCTCGCCCTTGCTCACC
ATGGTGGCGAATTCGAAGCTTGAGCACGAGATCTGAGTCCGGTAG
GCCTAGCGGATCTGACGGTTCACTAAACCAGCTCTGCTTATATAG
ACCTCCCACCGTACACGCCTACCGCCCATTTGCGTCAATGGGGCG
GAGTTGTTACGACATTTTGGAAAGTCCCGTTGATTTTGGTGCCAA
AACAAACTCCCATTGACGTCAATGGGGTGGAGACTTGGAAATCCC
CGTGAGTCAAACCGCTATCCACGCCCATTGATGTACTGCCAAAAC
CGCATCACCATGGTAATAGCGATGACTAATACGTAGATGTACTGC
CAAGTAGGAAAGTCCCATAAGGTCATGTACTGGGCATAATGCCAG
GCGGGCCATTTACCGTCATTGACGTCAATAGGGGGCGTACTTGGC
ATATGATACACTTGATGTACTGCCAAGTGGGCAGTTTACCGTAAA
TACTCCACCCATTGACGTCAATGGAAAGTCCCTATTGGCGTTACT
ATGGGAACATACGTCATTATTGACGTCAATGGGCGGGGGTCGTTG
GGCGGTCAGCCAGGCGGGCCATTTACCGTAAGTTATGTAACGGGC
CTGCTGCCGGCTCTGCGGCCTCTTCCGCGTCTTCGCCTTCGCCCT
CAGACGAGTCGGATCTCCCTTTGGGCCGCCTCCCCGCCTGTCTAG
CTTGACTGACTGAGATACAGCGTACCTTCAGCTCACAGACATGAT
AAGATACATTGATGAGTTTGGACAAACCACAACTAGAATGCAGTG
AAAAAAATGCTTTATTTGTGAAATTTGTGATGCTATTGCTTTATT
TGTAACCATTATAAGCTGCAATAAACAAGTTAACAACAACAATTG
CATTCATTTTATGTTTCAGGTTCAGGGGGAGGTGTGGGAGGTTTT
TTAAAGCAAGTAAAACCTCTACAAATGTGGTATTGGCCCATCTCT
ATCGGTATCGTAGCATAACCCCTTGGGGCCTCTAAACGGGTCTTG
AGGGGTTTTTTGTGCCCCTCGGGCCGGATTGCTATCTACCGGCAT
TGGCGCAGAAAAAAATGCCTGATGCGACGCTGCGCGTCTTATACT
CCCACATATGCCAGATTCAGCAACGGATACGGCTTCCCCAACTTG
CCCACTTCCATACGTGTCCTCCTTACCAGAAATTTATCCTTAAGG
TCGTCAGCTATCCTGCAGGCGATCTCTCGATTTCGATCAAGACAT
TCCTTTAATGGTCTTTTCTGGACACCACTAGGGGTCAGAAGTAGT
TCATCAAACTTTCTTCCCTCCCTAATCTCATTGGTTACCTTGGGC
TATCGAAACTTAATTAAGCGATCTGCATCTCAATTAGTCAGCAAC
CATAGTCCCGCCCCTAACTCCGCCCATCCCGCCCCTAACTCCGCC
CAGTTCCGCCCATTCTCCGCCCCATCGCTGACTAATTTTTTTTAT
TTATGCAGAGGCCGAGGCCGCCTCGGCCTCTGAGCTATTCCAGAA
GTAGTGAGGAGGCTTTTTTGGAGGCCTAGGCTTTTGCAAAGGAGG
TAGCCAACATGATTGAACAAGATGGATTGCACGCAGGTTCTCCCG
CCGCTTGGGTGGAGAGGCTATTCGGCTATGACTGGGCACAACAGA
CAATCGGCTGCTCTGATGCCGCCGTGTTCCGGCTGTCAGCGCAGG
GGCGCCCGGTTCTTTTTGTCAAGACCGACCTGTCCGGTGCCCTGA
ATGAACTCCAGGACGAGGCAGCGCGGCTATCGTGGCTGGCCACGA
CGGGCGTTCCTTGCGCAGCTGTGCTCGACGTTGTCACTGAAGCGG
GAAGGGACTGGCTGCTATTGGGCGAAGTGCCGGGGCAGGATCTCC
TGTCATCTCACCTTGCTCCTGCCGAGAAAGTATCCATCATGGCTG
ATGCAATGCGGCGGCTGCATACGCTTGATCCGGCTACCTGCCCAT
TCGACCACCAAGCGAAACATCGCATCGAGCGAGCACGTACTCGGA
TGGAAGCCGGTCTTGTCGATCAGGATGATCTGGACGAAGAGCATC
AGGGGCTCGCGCCAGCCGAACTGTTCGCCAGGCTCAAGGCGCGGA
TGCCCGACGGCGAGGATCTCGTCGTGACCCACGGCGATGCCTGCT
TGCCGAATATCATGGTGGAAAATGGCCGCTTTTCTGGATTCATCG
ACTGTGGCCGGCTGGGTGTGGCGGACCGCTATCAGGACATAGCGT
TGGCTACCCGTGATATTGCTGAAGAGCTTGGCGGCGAATGGGCTG
ACCGCTTCCTCGTGCTTTACGGTATCGCCGCTCCCGATTCGCAGC
GCATCGCCTTCTATCGCCTTCTTGACGAGTTCTTCTAGTATGTAA
GCCCTGTGCCTTCTAGTTGCCAGCCATCTGTTGTTTGCCCCTCCC
CCGTGCCTTCCTTGACCCTGGAAGGTGCCACTCCCACTGTCCTTT
CCTAATAAAATGAGGAAATTGCATCGCATTGTCTGAGTAGGTGTC
ATTCTATTCTGGGGGGTGGGGTGGGGCAGGACAGCAAGGGGGAGG
ATTGGGAAGACAATAGCAGGCATGCTGGGGATGCGGTGGGCTCTA
TGGTTAATTAACCAGTCAAGTCAGCTACTTGGCGAGATCGACTTG
TCTGGGTTTCGACTACGCTCAGAATTGCGTCAGTCAAGTTCGATC
TGGTCCTTGCTATTGCACCCGTTCTCCGATTACGAGTTTCATTTA
AATCATGTGAGCAAAAGGCCAGCAAAAGGCCAGGAACCGTAAAAA
GGCCGCGTTGCTGGCGTTTTTCCATAGGCTCCGCCCCCCTGACGA
GCATCACAAAAATCGACGCTCAAGTCAGAGGTGGCGAAACCCGAC
AGGACTATAAAGATACCAGGCGTTTCCCCCTGGAAGCTCCCTCGT
GCGCTCTCCTGTTCCGACCCTGCCGCTTACCGGATACCTGTCCGC
CTTTCTCCCTTCGGGAAGCGTGGCGCTTTCTCATAGCTCACGCTG
TAGGTATCTCAGTTCGGTGTAGGTCGTTCGCTCCAAGCTGGGCTG
TGTGCACGAACCCCCCGTTCAGCCCGACCGCTGCGCCTTATCCGG
TAACTATCGTCTTGAGTCCAACCCGGTAAGACACGACTTATCGCC
ACTGGCAGCAGCCACTGGTAACAGGATTAGCAGAGCGAGGTATGT
AGGCGGTGCTACAGAGTTCTTGAAGTGGTGGCCTAACTACGGCTA
CACTAGAAGAACAGTATTTGGTATCTGCGCTCTGCTGAAGCCAGT
TACCTTCGGAAAAAGAGTTGGTAGCTCTTGATCCGGCAAACAAAC
CACCGCTGGTAGCGGTGGTTTTTTTGTTTGCAAGCAGCAGATTAC
GCGCAGAAAAAAAGGATCTCAAGAAGATCCTTTGATCTTTTCTAC
GGGGTCTGACGCTCAGTGGAACGAAAACTCACGTTAAGGGATTTT
GGTCATGAGATTATCAAAAAGGATCTTCACCTAGATCCTTTTAAA
TTAAAAATGAAGTTTTAAATCAATCTAAAGTATATATGAGTAAAC
TTGGTCTGACAGTTACCAATGCTTAATCAGTGAGGCACCTATCTC
AGCGATCTGTCTATTTCGTTCATCCATAGTTGCATTTAAATTTCC
GAACTCTCCAAGGCCCTCGTCGGAAAATCTTCAAACCTTTCGTCC
GATCCATCTTGCAGGCTACCTCTCGAACGAACTATCGCAAGTCTC
TTGGCCGGCCTTGCGCCTTGGCTATTGCTTGGCAGCGCCTATCGC
CAGGTATTACTCCAATCCCGAATATCCGAGATCGGGATCACCCGA
GAGAAGTTCAACCTACATCCTCAATCCCGATCTATCCGAGATCCG
AGGAATATCGAAATCGGGGCGCGCCTGGTGTACCGAGAACGATCC
TCTCAGTGCGAGTCTCGACGATCCATATCGTTGCTTGGCAGTCAG
CCAGTCGGAATCCAGCTTGGGACCCAGGAAGTCCAATCGTCAGAT
ATTGTACTCAAGCCTGGTCACGGCAGCGTACCGATCTGTTTAAAC
CTAGATATTGATAGTCTGATCGGTCAACGTATAATCGAGTCCTAG
CTTTTGCAAACATCTATCAAGAGACAGGATCAGCAGGAGGCTTTC
GCATGAGTATTCAACATTTCCGTGTCGCCCTTATTCCCTTTTTTG
CGGCATTTTGCCTTCCTGTTTTTGCTCACCCAGAAACGCTGGTGA
AAGTAAAAGATGCTGAAGATCAGTTGGGTGCGCGAGTGGGTTACA
TCGAACTGGATCTCAACAGCGGTAAGATCCTTGAGAGTTTTCGCC
CCGAAGAACGCTTTCCAATGATGAGCACTTTTAAAGTTCTGCTAT
GTGGCGCGGTATTATCCCGTATTGACGCCGGGCAAGAGCAACTCG
GTCGCCGCATACACTATTCTCAGAATGACTTGGTTGAGTATTCAC
CAGTCACAGAAAAGCATCTTACGGATGGCATGACAGTAAGAGAAT
TATGCAGTGCTGCCATAACCATGAGTGATAACACTGCGGCCAACT
TACTTCTGACAACGATTGGAGGACCGAAGGAGCTAACCGCTTTTT
TGCACAACATGGGGGATCATGTAACTCGCCTTGATCGTTGGGAAC
CGGAGCTGAATGAAGCCATACCAAACGACGAGCGTGACACCACGA
TGCCTGTAGCAATGGCAACAACCTTGCGTAAACTATTAACTGGCG
AACTACTTACTCTAGCTTCCCGGCAACAGTTGATAGACTGGATGG
AGGCGGATAAAGTTGCAGGACCACTTCTGCGCTCGGCCCTTCCGG
CTGGCTGGTTTATTGCTGATAAATCTGGAGCCGGTGAGCGTGGGT
CTCGCGGTATCATTGCAGCACTGGGGCCAGATGGTAAGCCCTCCC
GTATCGTAGTTATCTACACGACGGGGAGTCAGGCAACTATGGATG
AACGAAATAGACAGATCGCTGAGATAGGTGCCTCACTGATTAAGC
ATTGGTAACCGATTCTAGGTGCATTGGCGCAGAAAAAAATGCCTG
ATGCGACGCTGCGCGTCTTATACTCCCACATATGCCAGATTCAGC
AACGGATACGGCTTCCCCAACTTGCCCACTTCCATACGTGTCCTC
CTTACCAGAAATTTATCCTTAAGATCGTTTAAACTCGACTCTGGC
TCTATCGAATCTCCGTCGTTTCGAGCTTACGCGAACAGCCGTGGC
GCTCATTTGCTCGTCGGGCATCGAATCTCGTCAGCTATCGTCAGC
TTACCTTTTTGGCAGCGATCGCGGCTCCCGACATCTTGGACCATT
AGCTCCACAGGTATCTTCTTCCCTCTAGTGGTCATAACAGCAGCT
TCAGCTACCTCTCAATTCAAAAAACCCCTCAAGACCCGTTTAGAG
GCCCCAAGGGGTTATGCTATCAATCGTTGCGTTACACACACAAAA
AACCAACACACATCCATCTTCGATGGATAGCGATTTTATTATCTA
ACTGCTGATCGAGTGTAGCCAGATCTAGTAATCAATTACGGGGTC
ATTAGTTCATAGCCCATATATGGAGTTCCGCGTTACATAACTTAC
GGTAAATGGCCCGCCTGGCTGACCGCCCAACGACCCCCGCCCATT
GACGTCAATAATGACGTATGTTCCCATAGTAACGCCAATAGGGAC
TTTCCATTGACGTCAATGGGTGGAGTATTTACGGTAAACTGCCCA
CTTGGCAGTACATCAAGTGTATCATATGCCAAGTACGCCCCCTAT
TGACGTCAATGACGGTAAATGGCCCGCCTGGCATTATGCCCAGTA
CATGACCTTATGGGACTTTCCTACTTGGCAGTACATCTACGTATT
AGTCATCGCTATTACCATGCTGATGCGGTTTTGGCAGTACATCAA
TGGGCGTGGATAGCGGTTTGACTCACGGGGATTTCCAAGTCTCCA
CCCCATTGACGTCAATGGGAGTTTGTTTTGGCACCAAAATCAACG
GGACTTTCCAAAATGTCGTAACAACTCCGCCCCATTGACGCAAAT
GGGCGGTAGGCGTGTACGGTGGGAGGTCTATATAAGCAGAGCTGG
TTTAGTGAACCGTCAGATCAGATCTTTGTCGATCCTACCATCCAC
TCGACACACCCGCCAGCGGCCGC
(SEQ ID NO: 48)
LINE-1_ORF2-MCP_MS2_mRNA (SEQ ID NO: 49)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGTGAGACAGCCGTCAGACCATCAAGACTAGGA
AGAAACTGCATCAACTAATGAGCAAAATCACCAGCTAACATCATA
GTATACATGACCGGCTCTAACTCACATATCACCATCCTTACACTT
AACATTAACGGCCTCAACTCAGCTATCAAGCGCCATCGGCTGGCC
AGCTGGATCAAATCACAGGATCCAAGCGTTTGTTGCATCCAAGAG
ACCCACCTGACCTGTAGAGATACTCACCGCCTCAAGATCAAGGGA
TGGCGAAAGATTTATCAGGCGAACGGTAAGCAGAAGAAAGCCGGA
GTCGCAATTCTGGTCTCAGACAAGACGGATTTCAAGCCCACCAAA
ATTAAGCGTGATAAGGAAGGTCACTATATTATGGTGAAAGGCAGC
ATACAGCAGGAAGAACTTACCATATTGAACATCTACGCGCCAAAC
ACCGGCGCACCTCGCTTTATCAAACAGGTCCTGTCCGATCTGCAG
CGAGATCTGGATTCTCATACGTTGATTATGGGTGATTTCAATACA
CCATTGAGCACCCTGGATCGCAGCACCAGGCAAAAGGTAAATAAA
GACACGCAAGAGCTCAATAGCGCACTGCATCAGGCAGATCTCATT
GATATTTATCGCACTCTTCATCCTAAGAGTACCGAGTACACATTC
TTCAGCGCCCCACATCATACATACTCAAAGATCGATCATATCGTC
GGCTCAAAGGCTCTGCTGTCAAAGTGCAAGCGCACAGAGATAATT
ACAAATTACCTGTCAGATCATAGCGCGATCAAGCTCGAGCTGAGA
ATCAAGAACCTGACCCAGAGCCGGAGTACCACTTGGAAGCTTAAT
AACCTGCTGCTCAACGATTATTGGGTCCACAATGAGATGAAGGCA
GAGATTAAAATGTTCTTCGAAACAAATGAGAATAAGGATACTACC
TATCAAAACCTTTGGGATGCCTTTAAGGCCGTCTGCAGAGGCAAG
TTCATCGCCCTCAACGCCTATAAAAGAAAACAAGAGAGATCTAAG
ATCGATACTCTCACCTCTCAGCTGAAGGAGTTGGAGAAACAGGAA
CAGACCCACTCCAAGGCGTCAAGACGGCAGGAGATCACAAAGATT
CGCGCCGAGTTGAAAGAGATCGAAACCCAAAAGACTCTTCAGAAA
ATTAACGAGTCTCGTAGTTGGTTCTTCGAGCGGATTAATAAGATA
GACAGACCTCTGGCACGACTGATTAAGAAGAAGCGCGAAAAGAAC
CAGATTGATACCATCAAGAACGACAAGGGCGACATCACTACTGAC
CCGACCGAGATCCAGACCACTATTCGGGAGTATTATAAGCATTTG
TATGCTAACAAGCTTGAGAACCTGGAAGAGATGGACACTTTTCTG
GATACCTATACTCTGCCACGGCTTAATCAAGAGGAAGTCGAGTCC
CTCAACCGCCCAATTACAGGAAGCGAGATTGTGGCCATAATTAAC
TCCCTGCCGACAAAGAAATCTCCTGGTCCGGACGGGTTTACAGCT
GAGTTTTATCAACGGTATATGGAAGAGCTTGTACCGTTTCTGCTC
AAGCTCTTTCAGTCTATAGAAAAGGAAGGCATCTTGCCCAATTCC
TTCTACGAAGCTTCTATAATACTTATTCCCAAACCAGGACGCGAT
ACCACAAAGAAGGAAAACTTCCGGCCCATTAGTCTCATGAATATC
GACGCTAAAATATTGAACAAGATTCTCGCCAACAGAATCCAACAA
CATATTAAGAAATTGATACATCACGACCAGGTGGGGTTTATACCT
GGCATGCAGGGCTGGTTTAACATCCGGAAGAGTATTAACGTCATT
CAACACATTAATAGAGCTAAGGATAAGAATCATATGATCATCTCT
ATAGACGCGGAAAAGGCATTCGATAAGATTCAGCAGCCATTTATG
CTCAAGACTCTGAACAAACTCGGCATCGACGGAACATATTTTAAG
ATTATTCGCGCAATTTACGATAAGCCGACTGCTAACATTATCCTT
AACGGCCAAAAGCTCGAGGCCTTTCCGCTCAAGACTGGAACCCGC
CAAGGCTGTCCCCTCTCCCCGCTTTTGTTTAATATTGTACTCGAG
GTGCTGGCTAGGGCTATTCGTCAAGAGAAAGAGATTAAAGGGATA
CAGCTCGGGAAGGAAGAGGTCAAGCTTTCCTTGTTCGCCGATGAT
ATGATTGTGTACCTGGAGAATCCTATTGTGTCTGCTCAGAACCTT
CTTAAACTTATTTCTAACTTTAGCAAGGTCAGCGGCTATAAGATT
AACGTCCAGAAATCTCAGGCCTTTCTGTACACAAATAATCGACAG
ACCGAATCCCAGATAATGGGTGAGCTTCCGTTTGTCATAGCCAGC
AAAAGGATAAAGTATCTCGGAATCCAGCTGACACGAGACGTTAAA
GATTTGTTTAAGGAAAATTACAAGCCTCTCCTGAAAGAGATTAAG
GAAGATACTAATAAGTGGAAGAATATCCCCTGTTCATGGGTTGGC
AGAATCAACATAGTGAAGATGGCAATACTTCCTAAAGTGATATAT
CGCTTTAACGCCATCCCAATTAAACTGCCTATGACCTTCTTTACG
GAGCTCGAGAAAACAACCCTTAAATTTATATGGAATCAAAAGAGA
GCAAGAATAGCGAAGTCCATCTTGAGCCAGAAGAATAAGGCCGGT
GGGATTACTTTGCCTGATTTTAAGTTGTATTATAAAGCCACAGTA
ACTAAGACAGCCTGGTATTGGTATCAGAATAGAGACATCGACCAG
TGGAATCGGACCGAACCATCAGAGATAATGCCCCACATCTATAAT
TACCTTATATTCGATAAGCCAGAAAAGAATAAACAGTGGGGCAAA
GACAGCCTCTTCAACAAGTGGTGTTGGGAGAATTGGCTGGCCATA
TGCCGGAAACTCAAGCTCGACCCCTTTCTTACACCCTACACTAAA
ATCAACAGTAGGTGGATCAAGGACTTGAATGTCAAGCCAAAGACT
ATAAAGACACTGGAAGAGAATCTTGGGATCACAATACAAGATATA
GGCGTCGGCAAAGATTTTATGTCAAAGACGCCCAAGGCCATGGCC
ACTAAGGATAAGATTGATAAGTGGGACCTTATTAAGCTCAAAAGC
TTCTGTACTGCCAAGGAGACCACGATCAGAGTTAATAGGCAGCCC
ACTACATGGGAAAAGATTTTCGCCACTTATTCATCAGATAAGGGG
TTGATAAGCAGAATATATAACGAGCTGAAGCAGATCTACAAGAAG
AAAACGAATAATCCCATCAAGAAGTGGGCAAAAGATATGAACAGG
CATTTTAGCAAAGAGGATATCTACGCCGCGAAGAAGCATATGAAG
AAGTGTAGTTCAAGCTTGGCCATTCGTGAGATGCAGATTAAGACG
ACCATGCGATACCACCTTACCCCAGTGAGGATGGCAATTATCAAG
AAATCTGGCAATAATAGATGTTGGCGGGGCTGTGGCGAGATTGGC
ACCCTGCTCCATTGCTGGTGGGATTGCAAGCTGGTGCAGCCGCTT
TGGAAATCAGTCTGGCGCTTTCTGAGGGACCTCGAGCTTGAGATT
CCCTTCGATCCCGCAATTCCCTTGCTCGGAATCTATCCTAACGAA
TACAAGAGCTGTTGTTACAAGGATACGTGTACCCGGATGTTCATC
GCGGCCTTGTTTACGATAGCTAAGACGTGGAATCAGCCTAAGTGC
CCCACAATGATCGATTGGATCAAGAAAATGTGGCATATTTATACC
ATGGAGTATTACGCAGCAATTAAGAATGACGAATTTATTTCCTTC
GTTGGGACCTGGATGAAGCTGGAGACTATTATTCTGAGCAAGCTG
TCTCAGGAGCAAAAGACAAAGCATAGAATCTTCTCTCTCATTGGT
GGTAACGCTTCTAACTTTACTCAGTTCGTTCTCGTCGACAATGGC
GGAACTGGCGACGTGACTGTCGCCCCAAGCAACTTCGCTAACGGG
ATCGCTGAATGGATCAGCTCTAACTCGCGTTCACAGGCTTACAAA
GTAACCTGTAGCGTTCGTCAGAGCTCTGCGCAGAATCGCAAATAC
ACCATCAAAGTCGAGGTGCCTAAAGGCGCCTGGCGTTCGTACTTA
AATATGGAACTAACCATTCCAATTTTCGCCACGAATTCCGACTGC
GAGCTTATTGTTAAGGCAATGCAAGGTCTCCTAAAAGATGGAAAC
CCGATTCCCTCAGCAATCGCAGCAAACTCCGGCATCTACGCCATG
GCCAGCAACTTCACCCAGTTCGTGCTGGTGGACAACGGCGGCACC
GGCGACGTGACCGTGGCCCCCAGCAACTTCGCCAACGGCATCGCC
GAGTGGATCAGCAGCAACAGCAGAAGCCAGGCCTACAAGGTGACC
TGCAGCGTGAGACAGAGCAGCGCCCAGAACAGAAAGTACACCATC
AAGGTGGAGGTGCCCAAGGGCGCCTGGAGAAGCTACCTGAACATG
GAGCTGACCATCCCCATCTTCGCCACCAACAGCGACTGCGAGCTG
ATCGTGAAGGCCATGCAGGGCCTGCTGAAGGACGGCAACCCCATC
CCCAGCGCCATCGCCGCCAACAGCGGCATCTACGACTACAAAGAC
GATGACGACAAGTAAAGCAACCTACAAACGGGTGGAGGATCACCC
CACCCGACACTTCACAATCAAGGGGTACAATACACAAGGGTGGAG
GAACACCCCACCCTCCAGACACATTACACAGAAATCCAATCAAAC
AGAAGCACCATCAGGGCTTCTGCTACCAAATTTATCTCAAAAAAC
TACAACAAGGAATCACCATCAGGGATTCCCTGTGCAATATACGTC
AAACGAGGGCCACGACGGGAGGACGATCACGCCTCCCGAATATCG
GCATGTCTGGCTTTCGAATTCAGTGCGTGGAGCATCAGCCCACGC
AGCCAATCAGAGTCGAATACAAGTCGACTTTCGCGAAGAGCATCA
GCCTTCGCGCCATTCTTACACAAACCACACTCTCCCCTACAGGAA
CAGCATCAGCGTTCCTGCCCAGTACCCAACTCAAGAAAATTTATG
TCCCCATGCAGCATCAGCGCATGGGCCCCAAGAATACATCCCCAA
CAAAATCACATCCGAGCACCAACAGGGCTCGGAGTGTTGTTTCTT
GTCCAACTGGACAAACCCTCCATGGACCATCAGGCCATGGACTCT
CACCAACAAGACAAAAACTACTCTTCTCGAAGCAGCATCAGCGCT
TCGAAACACTCGAGCATACATTGTGCCTATTTCTTGGGTGGACGA
TCACGCCACCCATGCTCTCACGAATTTCAAAACACGGACAAGGAC
GAGCACCACCAGGGCTCGTCGTTCCACGTCCAATACGATTACTTA
CCTTTCGGGATCACGATCACGGATCCCGCAGCTACATCACTTCCA
CTCAGGACATTCAAGCATGCACGATCACGGCATGCTCCACAAGTC
TCAACCACAGAAACTACCAAATGGGTTCAGCACCAGCGAACCCAC
TCCTACCTCAAACCTCTTCCCACAAAACTGGCAAGCAGGATCACC
GCTTGCCCATTCCAACATACCAAATCAAAAACAATTACTGGTACA
GCATCAGCGTACCAGCCCACATCTCTCACTACTATCAAAAACCAA
ACCGTTCAGCAACAGCGAACGGTACACACGGAAAAATCAACTGGT
TTACAAATACGAAAGACGATCACGCTTTCGTCCAGCGCAAACTAT
TACGAAAAACATCCGACGGGAAGAGCAACAGCCTTCCCGCGGCGG
AAAACCTCACAAAAACACGACAAACGGATGCACGAACACGGCATC
CGCCGACAACCCACAAACTTACAACCAGGCAAACGGTGCAGGATC
ACCGCACCGTACATCAAACACCTCAGATCTCATGCTTCTAGAAGT
TGTCTCCTCCTGCACTGACTGACTGATACAATCGATTTCTGGATC
CGCAGGCCTAATCAACCTCTGGATTACAAAATTTGTGAAAGATTG
ACTGGTATTCTTAACTATGTTGCTCCTTTTACGCTATGTGGATAC
GCTGCTTTAATGCCTTTGTATCATGCTATTGCTTCCCGTATGGCT
TTCATTTTCTCCTCCTTGTATAAATCCTGGTTGCTGTCTCTTTAT
GAGGAGTTGTGGCCCGTTGTCAGGCAACGTGGCGTGGTGTGCACT
GTGTTTGCTGACGCAACCCCCACTGGTTGGGGCATTGCCACCACC
TGTCAGCTCCTTTCCGGGACTTTCGCTTTCCCCCTCCCTATTGCC
ACGGCGGAACTCATCGCCGCCTGCCTTGCCCGCTGCTGGACAGGG
GCTCGGCTGTTGGGCACTGACAATTCCGTGGTGTTGTCGGGGAAG
CTGACGTCCTTTCCATGGCTGCTCGCCTGTGTTGCCACCTGGATT
CTGCGCGGGACGTCCTTCTGCTACGTCCCTTCGGCCCTCAATCCA
GCGGACCTTCCTTCCCGCTGAGAGACACAAAAAATTCCAACACAC
TATTGCAATGAAAATAAATTTCCTTTATTAGCCAGAAGTCAGATG
CTCAAGGGGCTTCATGATGTCCCCATAATTTTTGGCAGAGGGAAA
AAGATCTCAGTGGTATTTGTGAGCCAGGGCATTGGCCTTCTGATA
GGCAGCCTGCACCTGAGGAGTGCGGCCGCTTTACTTGTACAGCTC
GTCCATGCCGAGAGTGATCCCGGCGGCGGTCACGAACTCCAGCAG
GACCATGTGATCGCGCTTCTCGTTGGGGTCTTTGCTCAGGGCGGA
CTGGGTGCTCAGGTAGTGGTTGTCGGGCAGCAGCACGGGGCCGTC
GCCGATGGGGGTGTTCTGCTGGTAGTGGTCGGCGAGCTGCACGCT
GCCGTCCTCGATGTTGTGGCGGATCTTGAAGTTCACCTTGATGCC
GTTCTTCTGCTTGTCGGCCATGATATAGACGTTGTGGCTGTTGTA
GTTGTACTCCAGCTTGTGCCCCAGGATGTTGCCGTCCTCCTTGAA
GTCGATGCCCTTCAGCTCGATGCGGTTCACCAGGGTGTCGCCCTC
GAACTTCACCTCGGCGCGGGTCTTGTAGTTGCCGTCGTCCTTGAA
GAAGATGGTGCGCTCCTGGACGTAGCCTTCGGGCATGGCGGACTT
GAAGAAGTCGTGCTGCTTCATGTGGTCGGGGTAGCGGCTGAAGCA
CTGCACGCCGTAGGTCAGGGTGGTCACGAGGGTGGGCCAGGGCAC
GGGCAGCTTGCCGGTGGTGCAGATGAACTTCAGGGTCAGCTTGCC
GTAGGTGGCATCGCCCTCGCCCTCGCCGGACACGCTGAACTTGTG
GCCGTTTACGTCGCCGTCCAGCTCGACCAGGATGGGCACCACCCC
GGTGAACAGCTCCTCGCCCTTGCTCACCATGGTGGCGGGATCTGA
CGGTTCACTAAACCAGCTCTGCTTATATAGACCTCCCACCGTACA
CGCCTACCGCCCATTTGCGTCAATGGGGCGGAGTTGTTACGACAT
TTTGGAAAGTCCCGTTGATTTTGGTGCCAAAACAAACTCCCATTG
ACGTCAATGGGGTGGAGACTTGGAAATCCCCGTGAGTCAAACCGC
TATCCACGCCCATTGATGTACTGCCAAAACCGCATCACCATGGTA
ATAGCGATGACTAATACGTAGATGTACTGCCAAGTAGGAAAGTCC
CATAAGGTCATGTACTGGGCATAATGCCAGGCGGGCCATTTACCG
TCATTGACGTCAATAGGGGGCGTACTTGGCATATGATACACTTGA
TGTACTGCCAAGTGGGCAGTTTACCGTAAATACTCCACCCATTGA
CGTCAATGGAAAGTCCCTATTGGCGTTACTATGGGAACATACGTC
ATTATTGACGTCAATGGGCGGGGGTCGTTGGGCGGTCAGCCAGGC
GGGCCATTTACCGTAAGTTATGTAACGGGCCTGCTGCCGGCTCTG
CGGCCTCTTCCGCGTCTTCGCCTTCGCCCTCAGACGAGTCGGATC
TCCCTTTGGGCCGCCTCCCCGCCTGTCTAGCTTGACTGACTGAGA
TACAGCGTACCTTCAGCTCACAGACATGATAAGATACATTGATGA
GTTTGGACAAACCACAACTAGAATGCAGTGAAAAAAATGCTTTAT
TTGTGAAATTTGTGATGCTATTGCTTTATTTGTAACCATTATAAG
CTGCAATAAACAAGTT (SEQ ID NO: 49)
LINE1 ORF2-minke mRNA GFP (SEQ ID NO: 50)
TAATACGACTCACTATAGGGAGAAGTACTGCCACCATGGGCAAGA
AGCAAAATCGCAAGACGGGGAATTCCAAGACACAATCCGCTAGCC
CACCACCTAAAGAGCGTTCTAGCTCCCCTGCTACTGAGCAGTCCT
GGATGGAAAACGACTTCGATGAACTCCGGGAAGAGGGATTTAGGC
GATCCAACTATTCAGAACTCCGCGAAGATATCCAGACAAAGGGGA
AGGAAGTCGAGAATTTCGAGAAGAACCTCGAGGAGTGCATCACCC
GTATCACAAACACTGAGAAATGTCTCAAAGAACTCATGGAACTTA
AGACAAAAGCCAGGGAGCTTCGAGAGGAGTGTCGGAGTCTGAGAT
CCAGGTGTGACCAGCTCGAGGAGCGCGTGAGCGCGATGGAAGACG
AGATGAACGAGATGAAAAGAGAGGGCAAATTCAGGGAGAAGCGCA
TTAAGAGGAACGAACAGAGTCTGCAGGAGATTTGGGATTACGTCA
AGAGGCCTAACCTGCGGTTGATCGGCGTCCCCGAGAGCGACGTAG
AAAACGGGACTAAACTGGAGAATACACTTCAAGACATCATTCAAG
AAAATTTTCCAAACCTGGCTCGGCAAGCTAATGTGCAAATCCAAG
AGATCCAACGCACACCCCAGCGGTATAGCTCTCGGCGTGCCACCC
CTAGGCATATTATCGTGCGCTTTACTAAGGTGGAGATGAAAGAGA
AGATGCTGCGAGCCGCTCGGGAAAAGGGAAGGGTGACTTTGAAGG
GCAAACCTATTCGGCTGACGGTTGACCTTAGCGCCGAGACACTCC
AGGCACGCCGGGAATGGGGCCCCATCTTTAATATCCTGAAGGAGA
AGAACTTCCAGCCACGAATCTCTTACCCTGCAAAGTTGAGTTTTA
TCTCCGAGGGTGAGATTAAGTATTTCATCGATAAACAGATGCTGC
GAGACTTCGTGACAACTCGCCCAGCTCTCAAGGAACTGCTCAAAG
AGGCTCTTAATATGGAGCGCAATAATAGATATCAACCCTTGCAGA
ACCACGCAAAGATGTGAGACAGCCGTCAGACCATCAAGACTAGGA
AGAAACTGCATCAACTAATGAGCAAAATCACCAGCTAACATCATA
GTATACATGGTCATAGGAACTTACATTTCGATTATTACCTTAAAC
GTGAATGGGTTAAATGCCCCAACCAAGAGACATCGGCTGGCTGAA
TGGATTCAGAAACAGGACCCCTATATTTGCTGTCTGCAGGAGACC
CACTTCCGTCCTCGCGACACATACAGACTGAAAGTGAGGGGCTGG
AAAAAGATCTTCCATGCCAATGGAAATCAAAAGAAAGCTGGAGTG
GCTATTCTCATCTCAGATAAAATTGACTTCAAAATAAAGAATGTT
ACTCGAGATAAGGAGGGACACTACATAATGATCCAGGGGTCCATC
CAAGAAGAGGATATAACTATTATTAATATTTATGCACCCAACATT
GGCGCCCCTCAGTACATCAGGCAGCTGCTTACAGCTATCAAGGAG
GAAATCGACAGTAACACGATTATCGTGGGGGACTTTAACACCAGC
CTTACTCCGATGGATAGATCATCCAAAATGAAAATAAATAAGGAA
ACAGAGGCTCTTAATGACACCATTGACCAGATAGATCTGATTGAT
ATATATAGGACATTCCATCCAAAAACTGCCGATTACACTTTCTTC
AGCAGTGCGCATGGAACCTTCTCCAGGATAGATCACATCTTGGGT
CACAAAAGTAGCCTCAGTAAGTTTAAGAAAATTGAAATCATTAGC
AGCATCTTTTCTGACCATAACGCTATGCGCCTGGAGATGAATCAC
AGGGAGAAGAACGTAAAGAAGACAAACACCTGGAGGCTGAACAAT
ACGCTGCTAAATAACCAAGAGATCACTGAGGAAATCAAACAGGAA
ATAAAAAAATACTTGGAGACAAATGACAATGAAAACACGACCACC
CAGAACTTGTGGGATGCAGCTAAAGCGGTTCTGAGAGGGAAGTTT
ATAGCTATTCAAGCCTACCTTAAGAAACAGGAAAAATCTCAAGTG
AACAATTTGACCTTACACCTAAAGAAACTGGAGAAGGAGGAGCAG
ACCAAACCCAAAGTGAGCAGGAGGAAAGAAATCATCAAGATCAGA
GCCGAAATCAATGAAATAGAAACTAAGAAGACAATTGCCAAGATC
AATAAAACTAAATCCTGGTTCTTTGAGAAGATCAACAAAATTGAT
AAGCCATTAGCCAGACTCATCAAGAAAAAGAGGGAGAGGACTCAG
ATCAATAAGATCAGAAATGAGAAAGGGGAAGTTACAACCGACACC
GCGGAGATTCAGAACATCCTGAGAGACTACTACAAGCAACTTTAT
GCCAATAAAATGGACAACCTGGAAGAAATGGACAAATTCCTGGAA
AGGTATAACCTTCCCCGGCTGAACCAGGAGGAGACTGAAAATATC
AACCGCCCAATCACAAGTAATGAGATTGAGACTGTGATTAAGAAT
CTTCCAACTAACAAAAGTCCCGGCCCCGATGGCTTCACAGGTGAA
TTCTATCAGACCTTTCGGGAGGAGTTGACACCCATCCTTCTCAAG
CTCTTCCAAAAAATTGCAGAGGAGGGCACACTCCCGAACTCATTC
TATGAGGCCACCATCACCCTGATCCCAAAGCCCGACAAGGACACT
ACAAAGAAAGAAAATTACCGACCAATTTCCCTGATGAATATCGAT
GCCAAGATCCTCAACAAAATCTTGGCAAACAGAATCCAGCAGCAC
ATTAAGAGGATCATACACCACGATCAGGTGGGCTTTATCCCGGGG
ATGCAAGGATTCTTCAATATCCGCAAATCAATCAATGTGATCCAC
CATATTAACAAGTTGAAGAAGAAGAACCATATGATCATCTCCATC
GATGCAGAGAAAGCTTTTGACAAAATTCAACACCCATTTATGATC
AAAACTCTCCAGAAGGTGGGCATCGAGGGGACCTACCTCAACATA
ATTAAGGCCATCTATGATAAGCCCACAGCCAACATCATTCTCAAT
GGTGAAAAGCTGAAGGCATTTCCTCTGCGGTCCGGAACGAGACAG
GGATGTCCTCTCTCTCCTCTTCTGTTCAACATCGTTCTGGAAGTC
CTAGCCACCGCTATCCGCGAGGAAAAGGAAATTAAAGGCATACAG
ATTGGAAAGGAAGAGGTAAAACTGTCTCTGTTTGCGGATGATATG
ATACTGTACATAGAGAATCCTAAAACTGCCACCCGGAAGCTGTTG
GAGCTAATTAATGAGTATGGTAAGGTCGCCGGTTACAAGATTAAT
GCTCAGAAGTCTCTTGCTTTCCTGTACACTAATGATGAAAAGTCT
GAACGGGAAATTATGGAGACACTCCCCTTTACCATTGCAACCAAA
CGTATTAAATACCTTGGCATTAACCTGCCTAAGGAGACAAAAGAC
CTGTATGCTGAAAACTATAAGACACTGATGAAAGAGATTAAAGAT
GATACCAACCGGTGGCGGGATATCCCATGTTCTTGGATTGGCAGA
ATCAACATTGTGAAGATGAGCATCCTGCCCAAGGCCATCTACAGA
TTCAATGCCATCCCTATCAAATTACCTATGGCATTTTTTACGGAG
CTGGAACAGATCATCTTAAAATTTGTGTGGCGCCACAAGCGGCCC
CGAATCGCCAAAGCGGTCTTGAGGCAGAAGAATGGCGCTGGGGGA
ATCCGACTCCCTGACTTCAGATTGTACTACAAAGCTACCGTCATC
AAGACAATCTGGTACTGGCACAAGAACAGAAACATCGATCAGTGG
AACAAGATCGAAAGCCCTGAGATTAACCCCCGCACCTATGGTCAA
CTGATCTATGACAAAGGGGGCAAGGATATACAATGGCGCAAGGAC
AGCCTCTTCAATAAGTGGTGCTGGGAAAACTGGACAGCCACCTGC
AAGCGTATGAAGCTGGAGTACTCCCTGACACCATACACAAAAATA
AACTCAAAGTGGATTCGAGACCTCAATATTCGGCTGGACACTATA
AAACTCCTGGAGGAGAACATTGGGCGTACACTCTTTGACATTAAT
CATAGCAAGATCTTTTTCGATCCCCCTCCTCGTGTAATGGAAATA
AAAACAAAAATAAACAAGTGGGATCTGATGAAACTTCAGAGCTTT
TGCACCGCAAAGGAGACCATAAACAAGACGAAGCGCCAACCCTCA
GAATGGGAGAAAATATTTGCGAATGAGTCTACGGACAAAGGCTTA
ATCTCCAAAATATATAAGCAGCTCATTCAGCTCAATATCAAGGAA
ACAAACACCCCGATCCAAAAGTGGGCAGAGGACCTAAATCGGCAT
TTCTCCAAGGAAGACATCCAGACGGCCACGAAGCACATGAAGCGA
TGCTCAACTTCCCTGATTATTCGCGAAATGCAGATCAAGACTACT
ATGCGCTATCACCTCACTCCTGTTCGGATGGGCATCATCCGGAAA
TCTACAAACAACAAGTGCTGGAGAGGGTGTGGCGAAAAGGGAACC
CTCTTGCATTGTTGGTGGGAGTGTAAGTTGATCCAGCCACTATGG
CGGACCATATGGAGGTTCCTTAAAAAACTGAAGATTGAGCTGCCA
TATGACCCAGCAATCCCACTGCTGGGCATATACCCGGAGAAAACC
GTGATTCAGAAAGACACTTGCACCCGAATGTTCATTGCAGCATTG
TTTACAATAGCCAGGTCATGGAAGCAGCCTAAGTGCCCCTCGACA
GACGAGTGGATCAAGAAGATGTGGTACATTTATACTATGGAATAT
TACAGCGCCATCAAACGCAACGAAATTGGGTCTTTTCTGGAGACG
TGGATGGATCTAGAGACTGTCATCCAGAGTGAGGTAAGTCAGAAA
GAGAAGAACAAATATCGTATTTTAACGCATATTTGTGGAACCTGG
AAGAATGGTACAGATGAGCCGGTCTGCCGAACCGAGATTGAGACC
CAGATGGACTACAAAGACGATGACGACAAGTGAAGCGCTTCTAGA
AGTTGTCTCCTCCTGCACTGACTGACTGATACAATCGATTTCTGG
ATCCGCAGGCCTAATCAACCTCTGGATTACAAAATTTGTGAAAGA
TTGACTGGTATTCTTAACTATGTTGCTCCTTTTACGCTATGTGGA
TACGCTGCTTTAATGCCTTTGTATCATGCTATTGCTTCCCGTATG
GCTTTCATTTTCTCCTCCTTGTATAAATCCTGGTTGCTGTCTCTT
TATGAGGAGTTGTGGCCCGTTGTCAGGCAACGTGGCGTGGTGTGC
ACTGTGTTTGCTGACGCAACCCCCACTGGTTGGGGCATTGCCACC
ACCTGTCAGCTCCTTTCCGGGACTTTCGCTTTCCCCCTCCCTATT
GCCACGGCGGAACTCATCGCCGCCTGCCTTGCCCGCTGCTGGACA
GGGGCTCGGCTGTTGGGCACTGACAATTCCGTGGTGTTGTCGGGG
AAGCTGACGTCCTTTCCATGGCTGCTCGCCTGTGTTGCCACCTGG
ATTCTGCGCGGGACGTCCTTCTGCTACGTCCCTTCGGCCCTCAAT
CCAGCGGACCTTCCTTCCCGCTGAGAGACACAAAAAATTCCAACA
CACTATTGCAATGAAAATAAATTTCCTTTATTAGCCAGAAGTCAG
ATGCTCAAGGGGCTTCATGATGTCCCCATAATTTTTGGCAGAGGG
AAAAAGATCTCAGTGGTATTTGTGAGCCAGGGCATTGGCCTTCTG
ATAGGCAGCCTGCACCTGAGGAGTGCGGCCGCTTTACTTGTACAG
CTCGTCCATGCCGAGAGTGATCCCGGCGGCGGTCACGAACTCCAG
CAGGACCATGTGATCGCGCTTCTCGTTGGGGTCTTTGCTCAGGGC
GGACTGGGTGCTCAGGTAGTGGTTGTCGGGCAGCAGCACGGGGCC
GTCGCCGATGGGGGTGTTCTGCTGGTAGTGGTCGGCGAGCTGCAC
GCTGCCGTCCTCGATGTTGTGGCGGATCTTGAAGTTCACCTTGAT
GCCGTTCTTCTGCTTGTCGGCCATGATATAGACGTTGTGGCTGTT
GTAGTTGTACTCCAGCTTGTGCCCCAGGATGTTGCCGTCCTCCTT
GAAGTCGATGCCCTTCAGCTCGATGCGGTTCACCAGGGTGTCGCC
CTCGAACTTCACCTCGGCGCGGGTCTTGTAGTTGCCGTCGTCCTT
GAAGAAGATGGTGCGCTCCTGGACGTAGCCTTCGGGCATGGCGGA
CTTGAAGAAGTCGTGCTGCTTCATGTGGTCGGGGTAGCGGCTGAA
GCACTGCACGCCGTAGGTCAGGGTGGTCACGAGGGTGGGCCAGGG
CACGGGCAGCTTGCCGGTGGTGCAGATGAACTTCAGGGTCAGCTT
GCCGTAGGTGGCATCGCCCTCGCCCTCGCCGGACACGCTGAACTT
GTGGCCGTTTACGTCGCCGTCCAGCTCGACCAGGATGGGCACCAC
CCCGGTGAACAGCTCCTCGCCCTTGCTCACCATGGTGGCGGGATC
TGACGGTTCACTAAACCAGCTCTGCTTATATAGACCTCCCACCGT
ACACGCCTACCGCCCATTTGCGTCAATGGGGCGGAGTTGTTACGA
CATTTTGGAAAGTCCCGTTGATTTTGGTGCCAAAACAAACTCCCA
TTGACGTCAATGGGGTGGAGACTTGGAAATCCCCGTGAGTCAAAC
CGCTATCCACGCCCATTGATGTACTGCCAAAACCGCATCACCATG
GTAATAGCGATGACTAATACGTAGATGTACTGCCAAGTAGGAAAG
TCCCATAAGGTCATGTACTGGGCATAATGCCAGGCGGGCCATTTA
CCGTCATTGACGTCAATAGGGGGCGTACTTGGCATATGATACACT
TGATGTACTGCCAAGTGGGCAGTTTACCGTAAATACTCCACCCAT
TGACGTCAATGGAAAGTCCCTATTGGCGTTACTATGGGAACATAC
GTCATTATTGACGTCAATGGGCGGGGGTCGTTGGGCGGTCAGCCA
GGCGGGCCATTTACCGTAAGTTATGTAACGGGCCTGCTGCCGGCT
CTGCGGCCTCTTCCGCGTCTTCGCCTTCGCCCTCAGACGAGTCGG
ATCTCCCTTTGGGCCGCCTCCCCGCCTGTCTAGCTTGACTGACTG
AGATACAGCGTACCTTCAGCTCACAGACATGATAAGATACATTGA
TGAGTTTGGACAAACCACAACTAGAATGCAGTGAAAAAAATGCTT
TATTTGTGAAATTTGTGATGCTATTGCTTTATTTGTAACCATTAT
AAGCTGCAATAAACAAGTT (SEQ ID NO: 50)

### Example 19. Enriching Stably Retrotransposed Cells

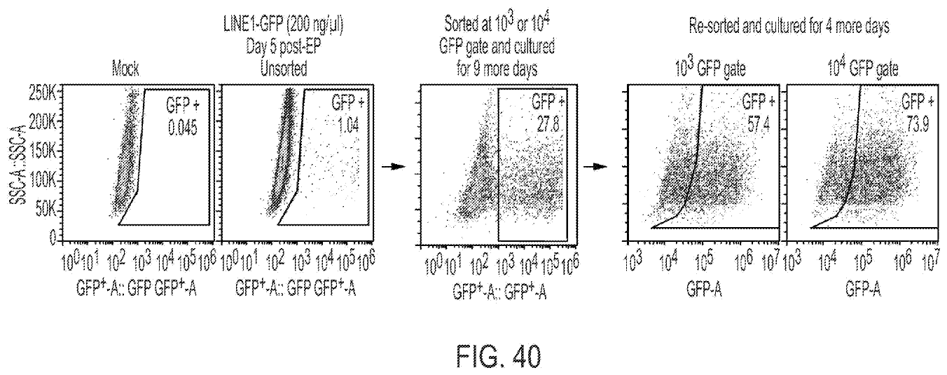

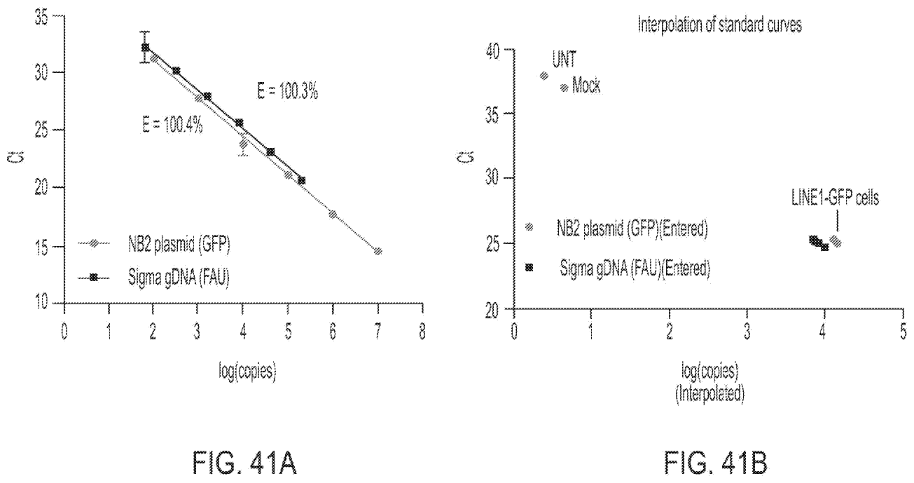

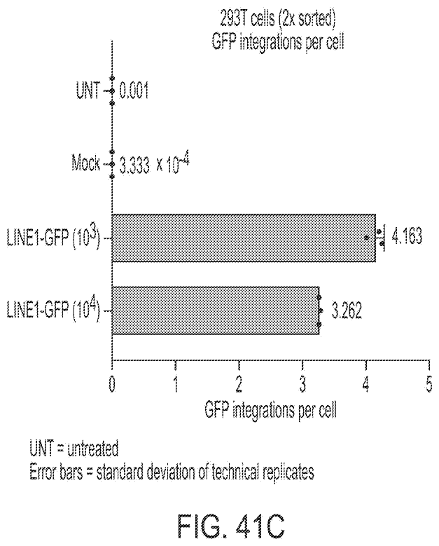

In an effort to increase the cell yield having stably integrated nucleic acid sequence a method of sorting and culturing was attempted, as described in this example. 293T cells were electroporated with LINE1-GFP mRNA produced by IVT and cultured in vitro for at least 3 days. Expression of GFP was determined periodically using flow cytometry, as shown in FIG. 40. Genomic integration per genome was evaluated using quantitative PCR. Interpolations of nucleic acid encoding GFP in the genome per genome were evaluated using standard curves for GFP and a housekeeping gene (FAU). In a sorting and enrichment culture of GFP positive cells, shown in FIG. 40, it was evident that integration was stable for multiple cell passages (at least 18 days post EP), and considerable enrichment was possible. GFP expression was detectable in ˜1% of 293T cells 5 days post-EP. GFP+ cells were enriched to ˜28% after first sorting and was further enriched up to ˜74% of cells after 2nd sorting. (FIG. 40, FIG. 41C).

Standard curves and exemplary quantitation of genomic integrations are shown in FIGS. 41A and 41B respectively. FIG. 41C shows average number of GFP integrations per genome when gated at 10\hat{ }3 units of GFP fluorescence intensity and at 1\hat{ }4 units of GFP fluorescence intensity.

### Example 20. Titration of mRNA concentration for increased transposon mediated integration

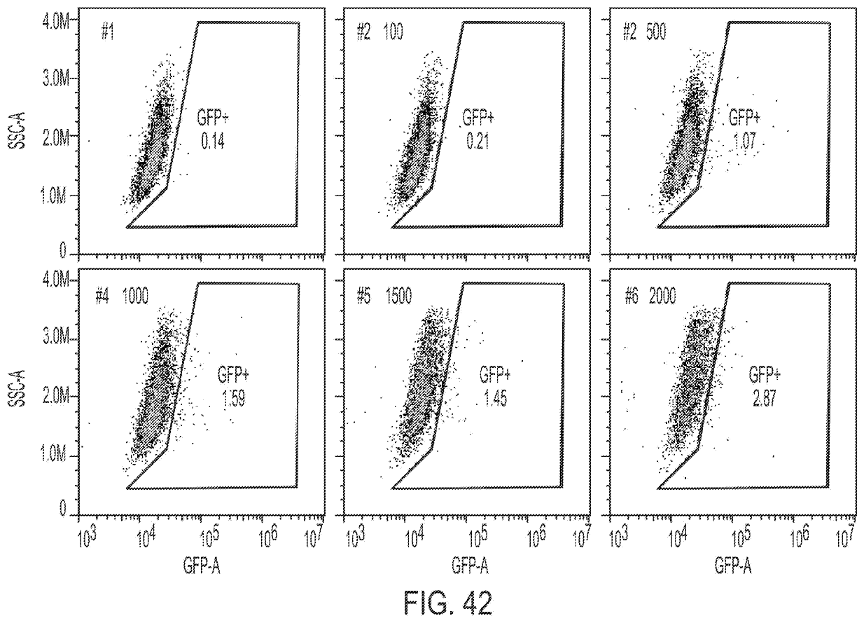

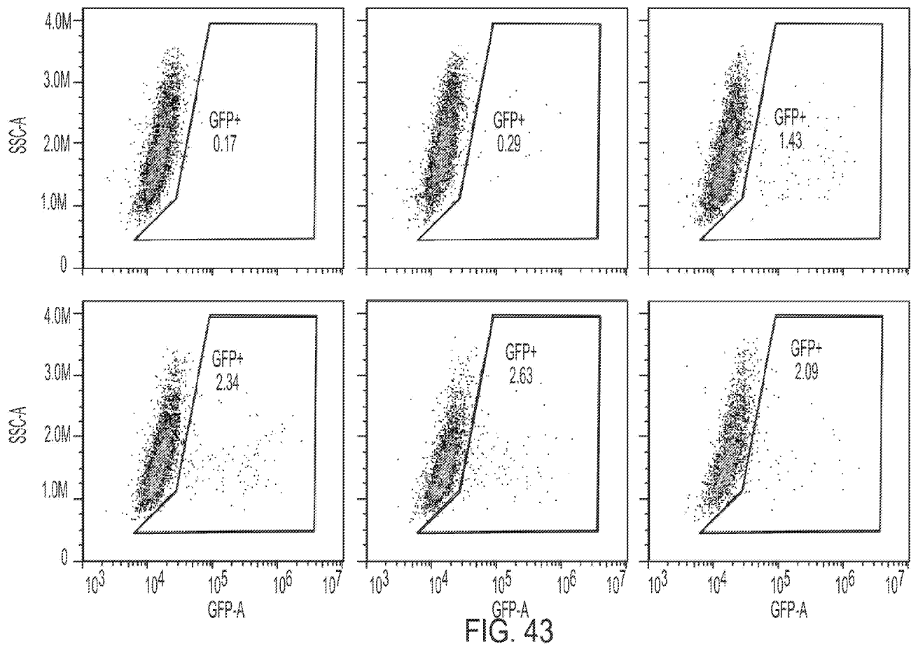

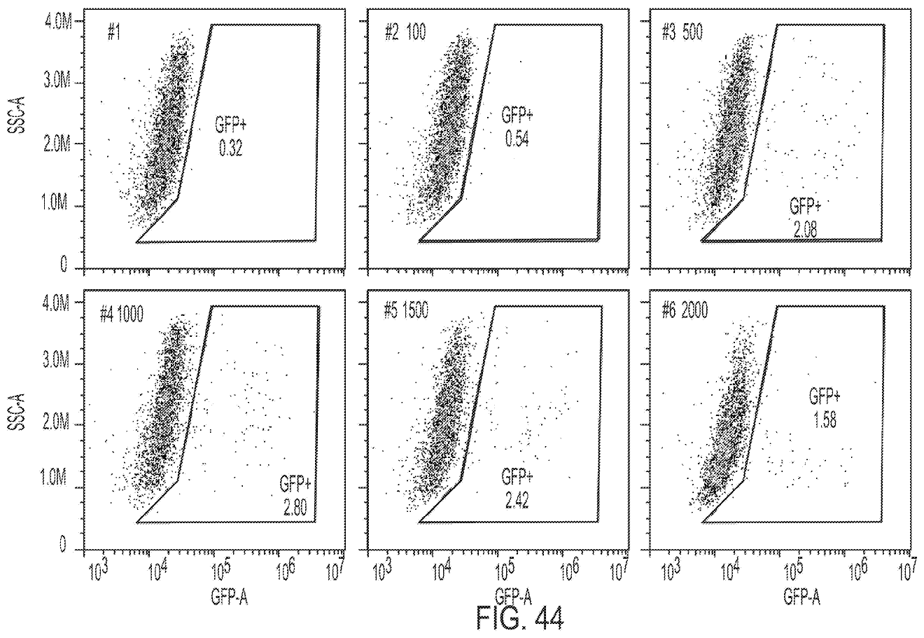

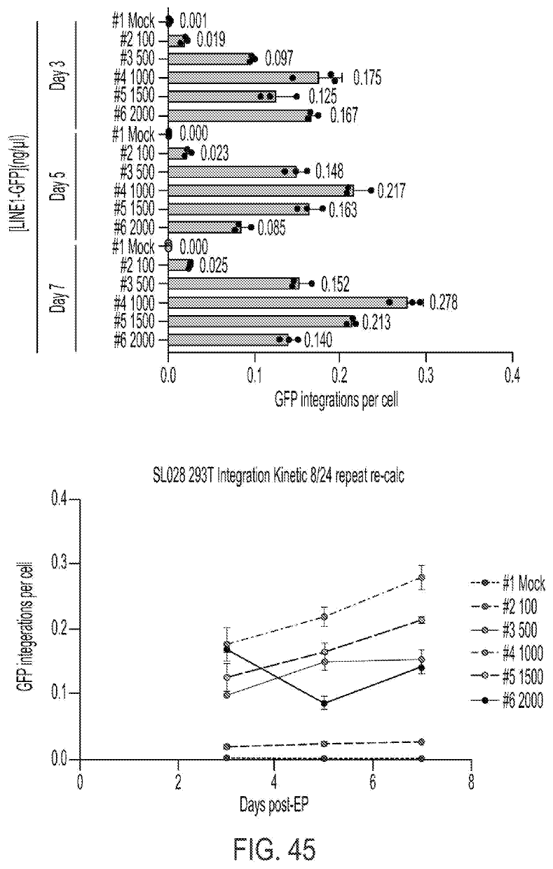

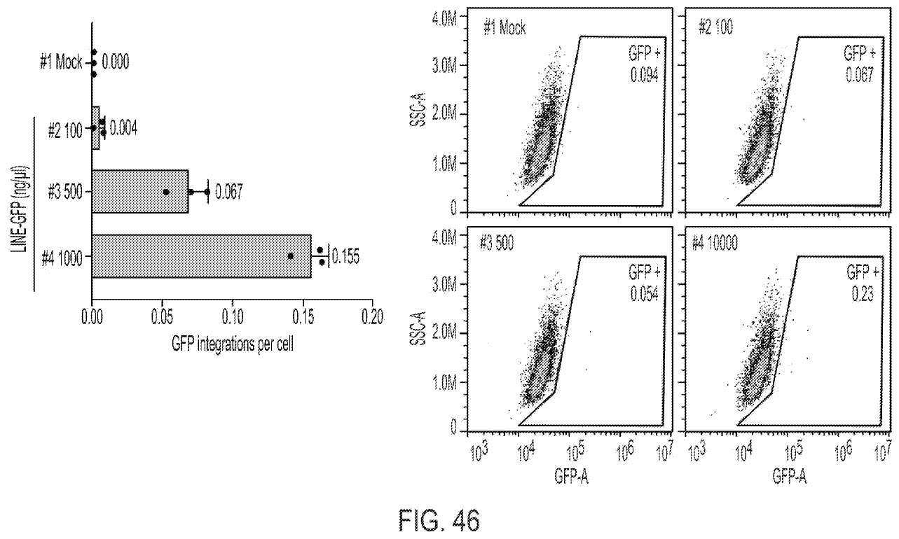

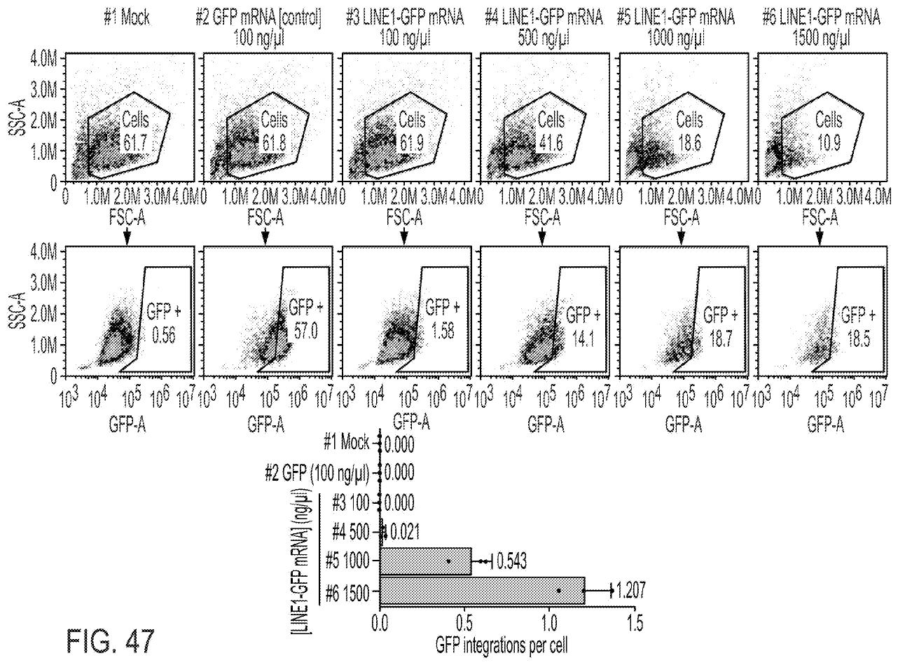

The concentration of LINE1-GFP mRNA used for electroporation was titrated for optimum genomic integration per cell in different cell types, 293T cells, K562 and THP-1 cells (FIGS. 42-46). 100, 500, 1000, 1500 and 2000 ng/μL of mRNA were tested for GFP expression and number of integrations per cell. Concentrations higher than 1000 ng/μl cause cell death. From the results shown in FIGS. 42, 43 and 44 that 1000 ng/μl causes a higher and long-term expression of GFP encoded by the retrotransposed integrated nucleic acid. Integrated DNA encoded protein expression starts to be detectable at day 3 and peaks around day 6-7 (FIG. 45). However, genomic integration and expression of the LINE-1 GFP mRNA in K562 and THP-1 was quite low; integration was detected at about 0.067-0.155 per cell in K562 cells (FIG. 46). (THP-1 data not shown). Higher LINE1-GFP mRNA concentrations (1500 and 2000 ng/μl) caused cell death in these cells. GFP mRNA expression in PD-0015 monocytes was detected at day 3 post electroporation, with detectable integration per cell. (FIG. 47). Steps were to be taken for more extensive DNase 1 treatment, and test mRNA batches were to be evaluated for residual plasmid before electroporation. Accuracy in determination of integration levels in the genome could be improved by first enriching for integrated DNA sequence by PCR followed by paired end sequencing leading to mapping the integration sites within the genome. Next generation sequencing is considered the gold standard in this respect, which involves gDNA extraction→shearing by sonication→DNA linkers ligated onto DNA ends→nested PCR (1: one primer for linker, second to integrated DNA, 2: Illumina sequencing adapters added)→paired-end sequencing.

### Example 21. Improvement of Integration Efficiency by Knockdown of Candidates that Prevent Transposon Mediated Integration

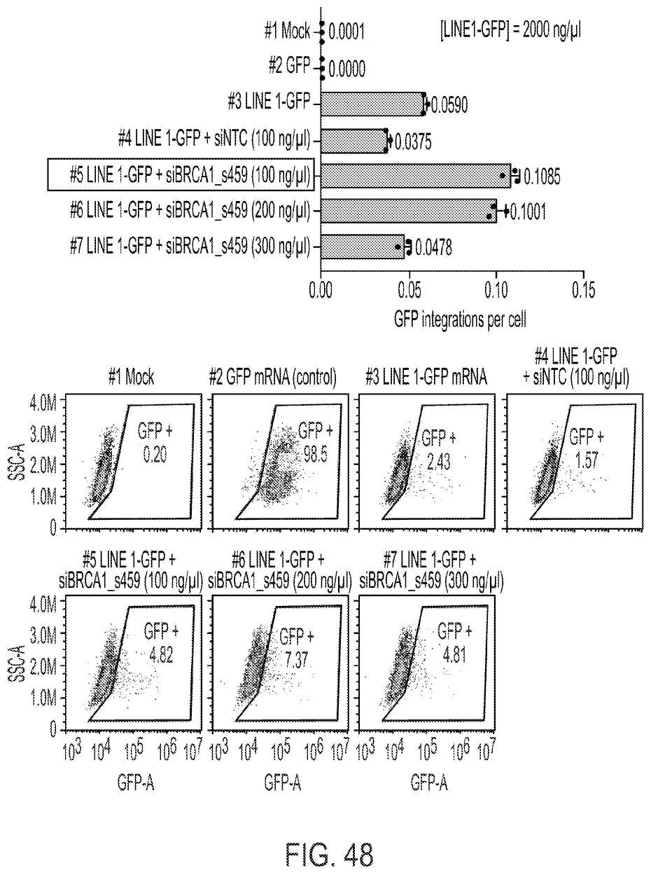

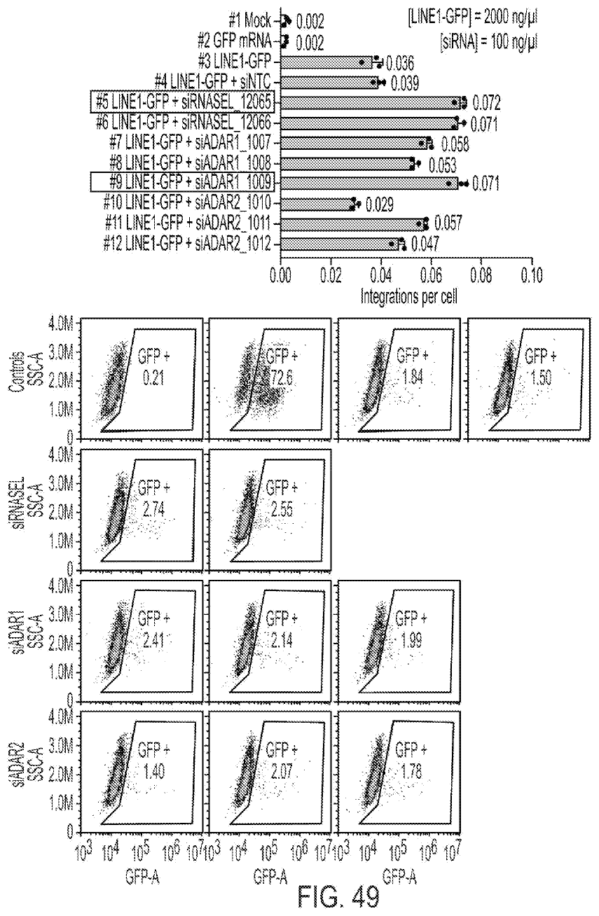

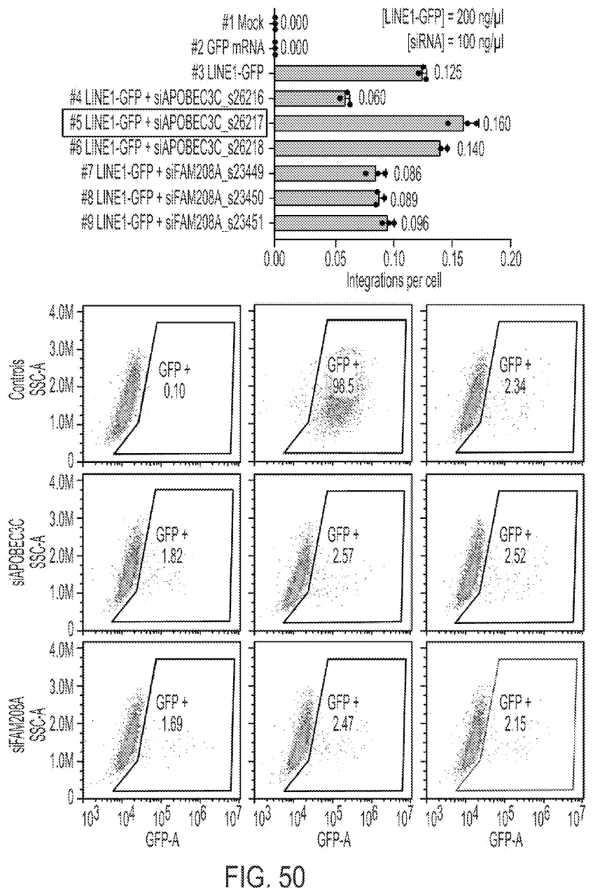

In this example, a number of endogenous candidates were knocked down using siRNA to determine if the knockdown could result in higher integration of test nucleic acid encoding GFP. Candidates included inhibitors of LINE1 retrotransposition: ADAR1, ADAR2 (ADAR1B), APOBEC3C, BRCA1, let-7 miRNA, RNase L, TASOR (HUSH complex), RAD51. siRNAs (3 per target candidate) were made, electroporated in test cells along with LINE 1-GFP mRNA and tested for alteration of the LINE-1 GFP expression by flow cytometry and its genome integration by qPCR and a cocktail of the siRNA that help increase LINE-1 GFP integration and expression was selected for further titration. Results from the different siRNAs tested are shown in FIGS. 48-51. Knockdown of ADAR1, BRCA and RNASEL tested individually induced about 2-fold increase in integration of LINE1-GFP. ADAR2, RAD51 and APOEBEC3C each led to less than 1.5-fold increase, and let7 miRNA and TASOR each led to no increase. In the study shown in FIG. 48, LINE-1 GFP (2000 ng/μL) was electroporated with an siBRCA at 100, 200 and 300 ng/μL in 293 cells, data shown at 4 days post electroporation. With 100 ng/μL, the integration rate was approximately ˜0.06 GFP copies per cell, and siBRCA1_s459 (100 ng/μl) increases integration by˜2-fold. Data shown in FIG. 49 demonstrates that at day 6 post electroporation, each of siRNASEL and siADAR1 siRNAs separately increased integration about 2-fold. On the other hand, siAPOBEC3C_s2617 increases GFP integration<1.5-fold (FIG. 50) at 6 days post electroporation.

TABLE 11
Effect of specific knockdowns on genomic integration rate.

| | GFP integration fold |
|---|---|
| Target | change in 293T cells |
| ADAR1 | ~2 fold increase |
| ADAR2 | <1.5-fold increase |
| APOEBEC3C | <1.5-fold increase |
| BRCA | ~2 fold increase |
| Let7 miRNA | No increase |
| RNASEL | ~2 fold increase |
| TASOR (Hush complex) | No increase |
| RAD51 | <1.5-fold increase |

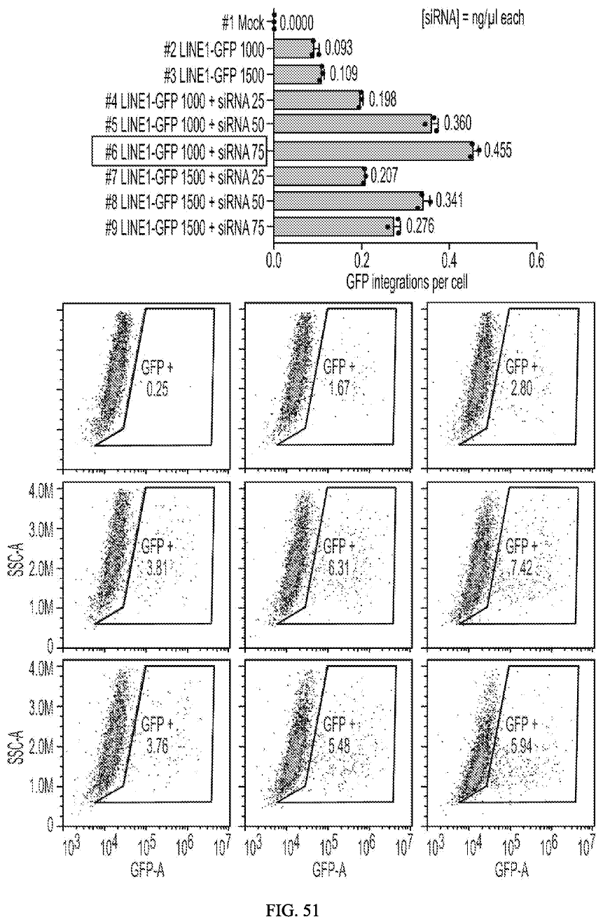

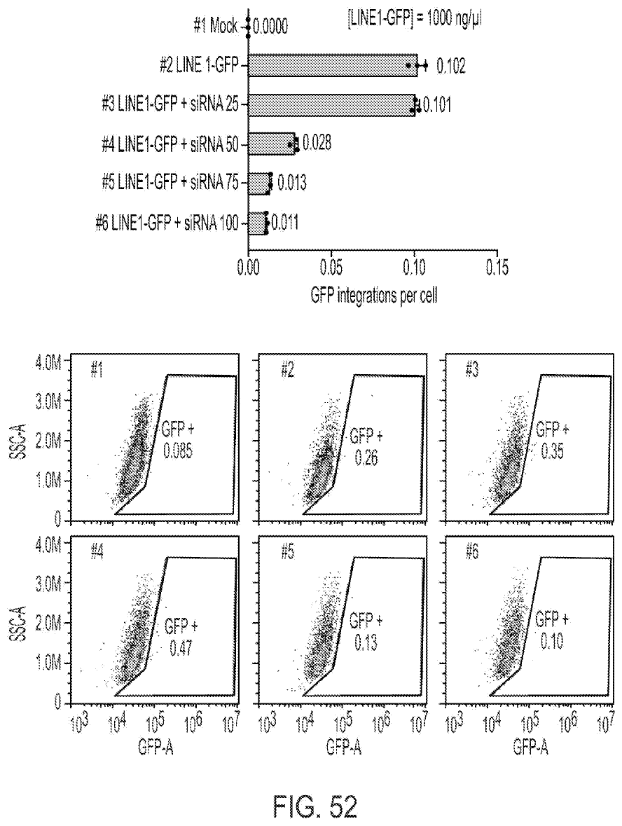

siRNA against ADAR, APOEBEC3C, BRCA and RNASEL were chosen for the siRNA cocktail. Using 1000 ng/μL and 1500 ng/μL LINE1-GFP mRNA in two sets of experiments, the concentration of the siRNAs for electroporation was titrated next. It was observed that LINE1-GFP mRNA at 1500 ng/μL was slightly toxic (FIG. 51). With 1000 ng/μL, 75 ng/uL of each siRNA resulted in ˜5-fold improvement of integration of GFP in 293T cells. These results were highly encouraging and support further development. Results from a similar experiment in K562 cells are shown in FIG. 52.

### Example 20. Improvement of Retrotransposition Efficiency

Efficiency of retrotransposition can be impacted by numerous variables, such as the level of retrotransposon gene integration and subsequent transcription and translation efficiency of an integrated gene. In this example, variables such as LINE-1 mutations, mRNA sequence alterations/additions, and alterations in mRNA chemistry, are tested for their impact on increasing retrotransposition efficiency and expression of the construct in a cell. To improve the cargo gene integration, the following exemplary experiments will be conducted to test their impact on increasing retrotransposition efficiency and expression of the construct in a cell: (1) mRNA modifications, such as those that increase mRNA stability and protein expression; (2) sequence modifications, such as addition of nuclear localization sequences (NLSs) and introducing mutations in LINE-1 proteins to enhance localization and integration; (3) methods to improve cargo expression and; (4) bioinformatic analysis on various retroelements from different organisms. The learnings from these experiments are adapted across a number of human cell types, including myeloid cells, T cells, hepatocytes, cardiomyocytes, neurons and retinal pigment epithelial cells, to determine the specificity and versatility of these approaches to different cell types. In vivo delivery of the retrotransposon will also be conducted in mice.

Exemplary cells included in these experiments include hepatocytes, cardiomyocytes, retinal pigment epithelial cells and neurons. Primary cells and cell lines will be used and can be cultured in conditions optimal for each cell type/cell line. Test constructs include plasmid constructs and mRNA constructs comprising a sequence encoding GFP along with a promoter and a poly A sequence that is inserted in reverse orientation relative to the ORF1/2 genes in the retrotransposon complex as described elsewhere in the specification. For plasmid constructs, test constructs include constructs that contain the GFP gene in an antisense orientation interrupted by an intron in sense orientation. For mRNA constructs, test constructs include constructs without an intron. Retrotransposition conditions can include reagents to enhance integration, with the GFP-retrotransposon delivered to the cells in vitro via electroporation and/or a transfection reagent. Electroporation conditions optimized for each specific cell type are used.

Efficiency of integration is determined by flow cytometry, for example using gates set up based on SSC and FSC. GFP is measured in negative control and positive control to set gates for flow cytometry. An exemplary negative control used will be set at <0.1% GFP+. An exemplary positive control used will be set at >90% GFP+. GFP is measured in negative control and positive control to set gates for flow cytometry. An exemplary negative control used will be set at <0.1% GFP+. An exemplary positive control used will be set at >90% GFP+. An exemplary measurement indicative of successful integration is measurement of GFP expression, as determined by flow cytometry, in >2% of cells by day 10 post retrotransposition. Another exemplary measurement indicative of successful integration is measurement of GFP expression, as determined by flow cytometry, in >10% of cells by day 10 post retrotransposition.

Additional assays such as PCR and next generation sequencing (NGS) are performed, for example, to confirm integration. Next generation sequencing can be performed on the transfected/electroporated cells.

Exemplary mRNA structures that may directly impact mRNA stability and translation efficiency to which modifications will be tested to increase stability and protein expression include the 5′ CAP, the poly(A) tail, and the untranslated regions (UTRs). For example, enzymatic incorporation of the 5′CAP and use of CAP analogies, such as 7-methylguanosine, will be tested. Enzymatic incorporation of the 5′CAP may be efficient than using CAP analogs, For example, variation of the poly(A) tail length will be also be tested to determine whether the poly(A) tail length impacts translation efficiency. For example, variation of the percent GC content of mRNA will be tested to determine whether the GC content impacts translation efficiency. High GC content may increase mRNA secondary structure stability. Low GC content may reduce translatability of the mRNA. Sequence-specific mRNA variations can be tested to arrive at an optimum GC content. In another exemplary method, substituting certain codons with rare codons and inserting modified nucleotides will be undertaken in order to determine if it can affect translation efficiency. Other exemplary nucleotide modifications that will be tested to increase stability and protein expression include use of 5-methylcytidine (m5C) and pseudouridine (W) nucleotides. An exemplary measurement indicative of successful integration is measurement of GFP expression, as determined by flow cytometry, in >2% of cells by day 10 post retrotransposition. Another exemplary measurement indicative of successful integration is measurement of GFP expression, as determined by flow cytometry, in >10% of cells by day 10 post retrotransposition.

Different mRNA production processes will also be tested for increased transcription of full-length mRNAs.

Exemplary NLS sequence modifications will be tested, such as for enhanced localization and integration, include various NLS sequences, placement of the NLS sequences upstream or downstream of LINE-1 elements and the number of NLS sequences used. As above, GFP is measured in negative control and positive control to set gates for flow cytometry. As above, an exemplary negative control used will be set at <0.1% GFP+. As above, an exemplary positive control used will be set at >90% GFP+.

An exemplary measurement indicative of successful cargo gene expression is measurement of increased GFP expression, as determined by flow cytometry, in >10% of cells by day 10 post retrotransposition.

Exemplary sequence modifications that encode mutations in LINE-1 proteins that will be tested, such as for enhanced localization and integration, include K3R of ORF1 protein and Y1 180A of ORF2.

As above, GFP is measured in negative control and positive control to set gates for flow cytometry. As above, an exemplary negative control used will be set at <0.1% GFP+. As above, an exemplary positive control used will be set at >90% GFP+. An exemplary measurement indicative of successful cargo gene expression is measurement of increased GFP expression, as determined by flow cytometry, in >10% of cells by day 10 post retrotransposition.

### Example 21. Effect of Introducing a Nuclear Localization Signal in Retrotransposition by Human LINE1 Constructs

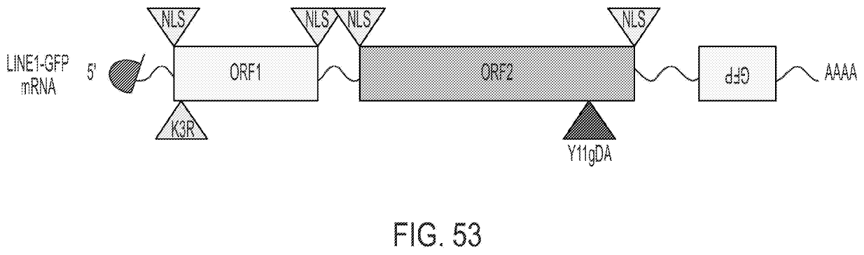

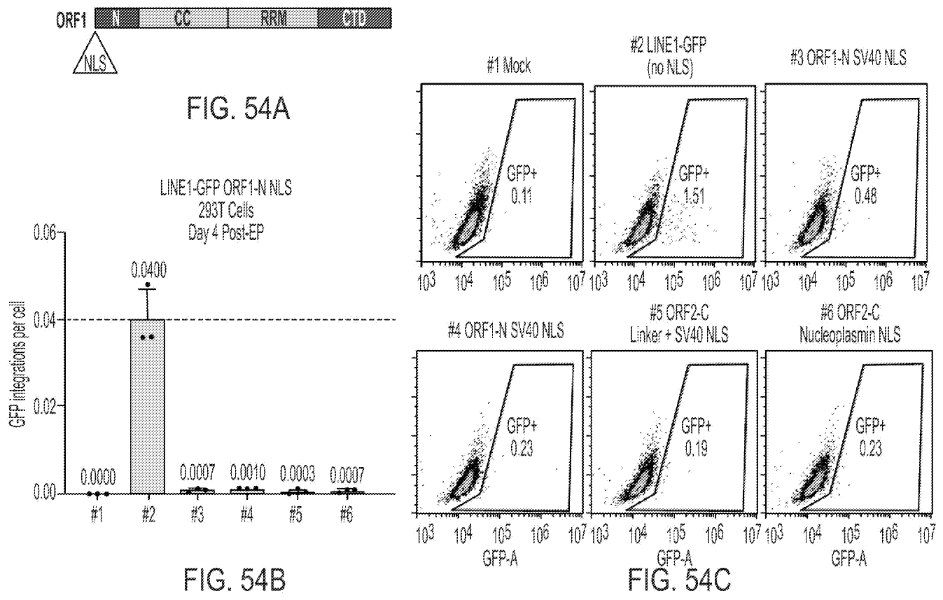

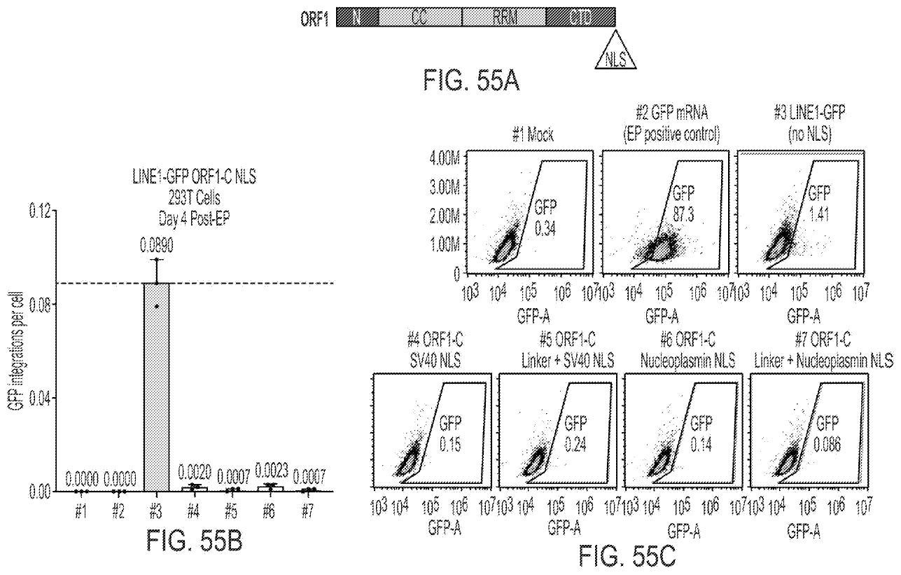

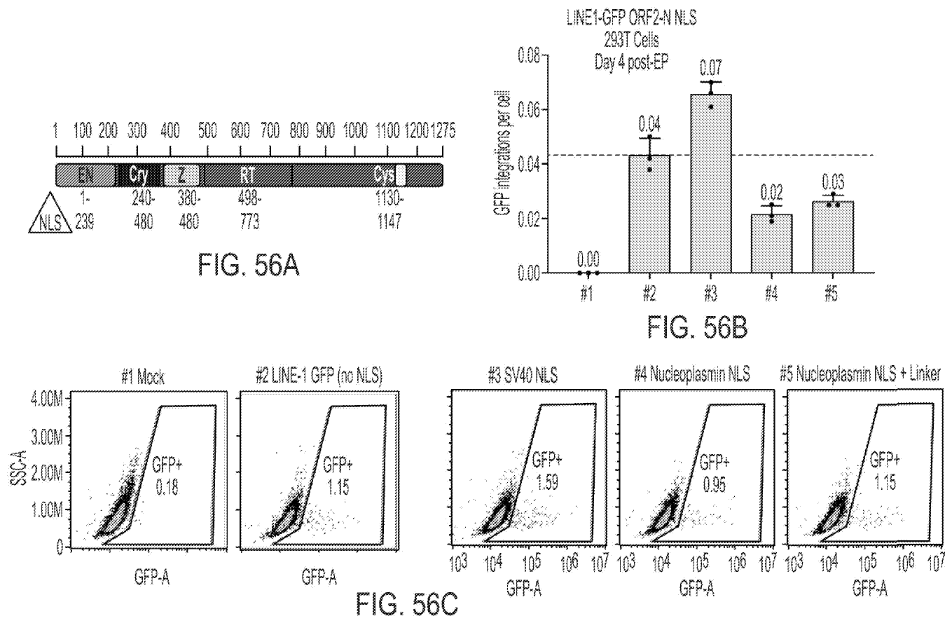

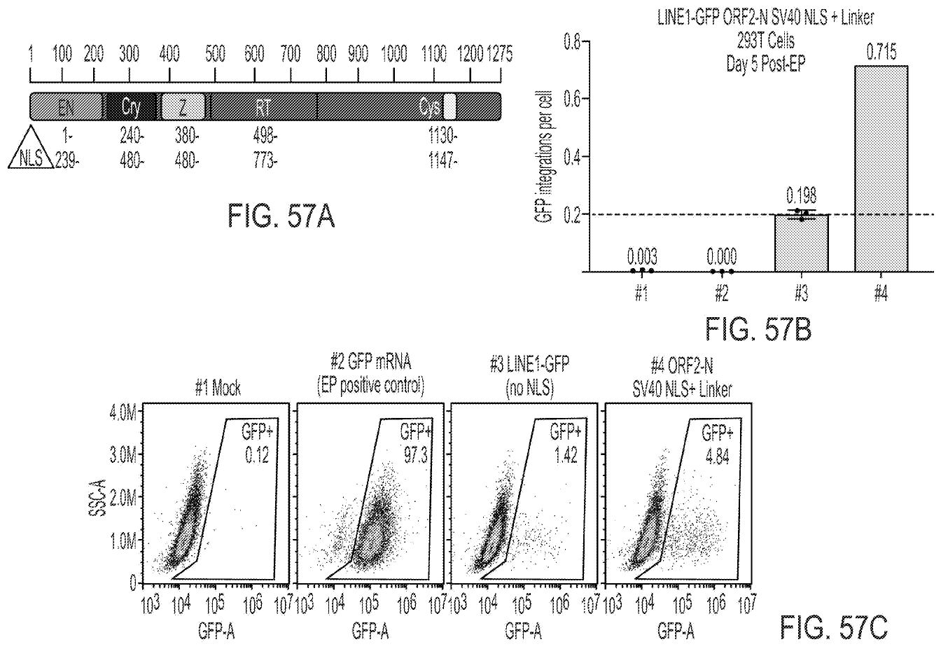

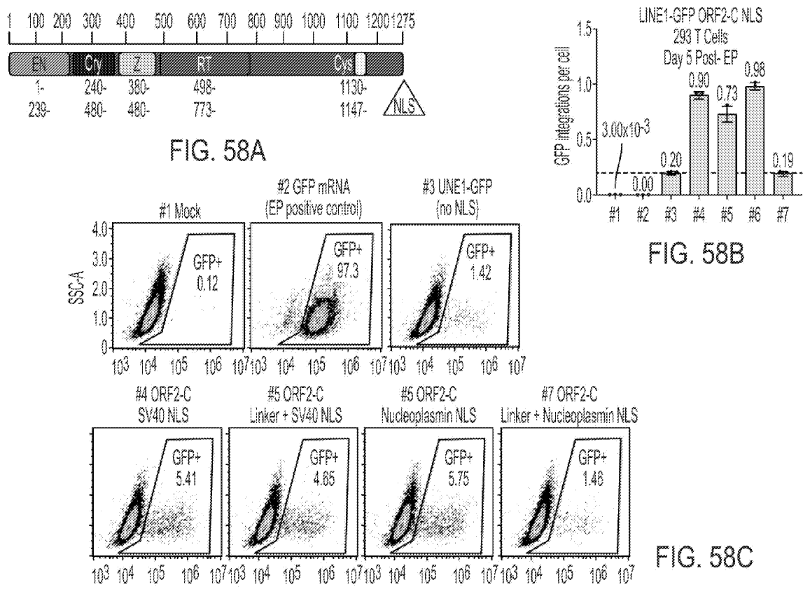

In this example, several constructs were generated and tested in which a nuclear localization signal was inserted in different locations as described below, and retrotransposition activity was tested in HEK293T cells. mRNA constructs containing one or more various NLSs and different numbers of total NLSs comprising the retrotransposon sequences described herein were electroporated in the 293T cells, and the number of insertions of a test gene in the genome of a cell was investigated. NLSs were individually tested at the following positions in LINE1-GFP mRNA as summarized in FIG. 53: (i) ORF1-N, in which an NLS was inserted at the N-terminal end of the sequence encoding ORF1 of a LINE1-GFP construct (FIG. 54A); (ii) ORF1-C, in which an NLS was inserted at the C-terminal end of the sequence encoding ORF1 of a LINE1-GFP construct (FIG. 55A), (iii) ORF2-N, in which an NLS was inserted at the N-terminal end of the sequence encoding ORF2 of a LINE1-GFP construct (FIG. 56A); (iv) ORF2-N, in which an SV40 NLS and a linker was inserted at the N-terminal endo of the sequence encoding ORF2 of a LINE 1-GFP construct (FIG. 57A); and (v) ORF2-C in which an NLS was inserted at the C-terminal end of the sequence encoding ORF2 of(a L SE1-GFP construct (FIG. 58A). In each case, retrotransposition using the LINE GFP NLS constructs was measured in number of GFP insertions per cell (genome).

Table 12A details sequences relevant to the constructs in tests described for FIGS. 54A-55C.

TABLE 12A

| NLS | Amino acid sequence and nucleic acid sequence |
|---|---|
| (#3) SV40 NLS | PKKKRKV (SEQ ID NO: 80) ccaaagaagaagcggaaggtc (SEQ ID NO: 81) |
| (#4) SV40 NLS + Linker | PKKKRKVGGGS (SEQ ID NO: 82) |
| (linker sequence underlined) | ccaaagaagaagcggaaggtcggcggcggcagc (SEQ ID NO: 83) |
| (#5) Nucleoplasmin NLS | KRPAATKKAGQAKKKK (SEQ ID NO: 84) |
| | aaaaggccggcggccacgaaaaaggccggccaggcaaaaaagaaaaag (SEQ ID NO: 85) |
| (#6) Nucleoplasmin NLS + | KRPAATKKAGQAKKKKGGGS (SEQ ID NO: 86) |
| Linker | aaaaggccggcggccacgaaaaaggccggccaggcaaaaaagaaaaagggcggcggcagc (SEQ ID |
| (linker sequence underlined) | NO: 87) |

As shown in FIGS. 54B-54C and FIGS. 55B-55C, NLS insertions at N-terminal and C-terminal of ORF1 sequence abolishes retrotransposition activity of the LINE-i constructs. On the other hand, each of the constructs shown in FIGS. 56A-58C showed some improvement in retrotransposition, with insertion of the NLS in either ORF2 N or C terminal.

Table 12B details sequences relevant to the constructs in tests described for FIGS. 56-56C.

TABLE 12B

| NLS | Amino acid sequence and nucleic acid sequence |
|---|---|
| (#3) SV40 NLS | PKKKRKV (SEQ ID NO: 80) |
| | ccaaagaagaagcggaaggtc (SEQ ID NO: 81) |
| (#4) Nucleoplasmin NLS | KRPAATKKAGQAKKKK (SEQ ID NO: 84) |
| | aaaaggccggcggccacgaaaaaggccggccaggcaaaaaagaaaaag (SEQ ID NO: 85) |
| (#5) Linker + | GGGSKRPAATKKAGQAKKKK (SEQ ID NO: 88) |
| Nucleoplasmin NLS (linker | ggcggcggcagcaaaaggccggcggccacgaaaaaggccggccaggcaaaaaagaaaaag (SEQ |
| sequence underlined) | ID NO: 89) |

With insertion of an NLS at the N terminal end of ORF2 sequence, about 2-fold increase in number of insertions per cell. (FIGS. 56B, and 56C). However, using the SV40 NLS with the addition of a linker sequence shown in Table 12C, increases the number of GFP integrations by about 3.5 folds (FIGS. 57B and 57C).

Table 12C details sequences relevant to the constructs in tests described for FIGS. 57-57C.

TABLE 12C

| NLS | Amino acid sequence and nucleic acid sequence |
|---|---|
| (#4a) SV40 NLS + Linker | PKKKRKVGGGS (SEQ ID NO: 82) |
| (linker sequence underlined) | ccaaagaagaagcggaaggtcggcggcggcagc (SEQ ID NO: 83) |

However, surprisingly, insertion of the NLS at the ORF2 C terminus increases the retrotransposition by about 5 fold (FIG. 58B-58C).

Table 12D details sequences relevant to the constructs in tests described for FIGS. 58A-58C.

TABLE 12D

| NLS | Amino acid sequence and nucleic acid sequence |
|---|---|
| (#3) SV40 NLS | PKKKRKV (SEQ ID NO: 80) |
| | ccaaagaagaagcggaaggtc (SEQ ID NO: 81) |
| (#4) Linker + SV40 | GGGSPKKKRKV (SEQ ID NO: 90) |
| NLS | ggcggcggcagcccaaagaagaagcggaaggtc (SEQ ID NO: 91) |
| (#5) Nucleoplasmin | KRPAATKKAGQAKKKK (SEQ ID NO: 84) |
| NLS | aaaaggccggcggccacgaaaaaggccggccaggcaaaaaagaaaaag (SEQ ID NO: 85) |
| (#6) Linker + | GGGSKRPAATKKAGQAKKKK (SEQ ID NO: 88) |
| Nucleoplasmin NLS | ggcggcggcagcaaaaggccggcggccacgaaaaaggccggccaggcaaaaaagaaaaag (SEQ ID NO: 89) |

Insertion of both the SV40 NLS and Nucleoplasmin NLS C-terminal to ORF2 leads to >5% GFP positive cells compared to less than 2% in cells that were electroporated with a LINE 1-GFP mRNA construct without an NLS sequence. These results indicate that the ORF2 modifications with NLS increases retrotransposition efficiency. Most significantly, insertion at the C-terminal provides higher increase in retrotransposition of LINE1 elements.

Exemplary plasmids used for these experiments are shown in Table 13.

TABLE 13
Exemplary plasmid sequences

| Name | Sequence |
|---|---|
| >RET- | CTTTCGTCCGATCCATCTTGCAGGCTACCTCTCGAACGAACTATCGCAAGTCTCTTGGCCGGCCTTG |
| 002 | CGCCTTGGCTATTGCTTGGCAGCGCCTATCGCCAGGTATTACTCCAATCCCGAATATCCGAGATCGG |
| LINE1- | GATCACCCGAGAGAAGTTCAACCTACATCCTCAATCCCGATCTATCCGAGATCCGAGGAATATCGAA |
| GFP | ATCGGGGCGCGCCTGGTGTACCGAGAACGATCCTCTCAGTGCGAGTCTCGACGATCCATATCGTTGC |
| ORF1- | TTGGCAGTCAGCCAGTCGGAATCCAGCTTGGGACCCAGGAAGTCCAATCGTCAGATATTGTACTCAA |
| CSV40- | GCCTGGTCACGGCAGCGTACCGATCTGTTTAAACCTAGATATTGATAGTCTGATCGGTCAACGTATA |
| NLS | ATCGAGTCCTAGCTTTTGCAAACATCTATCAAGAGACAGGATCAGCAGGAGGCTTTCGCATGAGTAT |
| | TCAACATTTCCGTGTCGCCCTTATTCCCTTTTTTGCGGCATTTTGCCTTCCTGTTTTTGCTCACCCA |
| | GAAACGCTGGTGAAAGTAAAAGATGCTGAAGATCAGTTGGGTGCGCGAGTGGGTTACATCGAACTGG |
| | ATCTCAACAGCGGTAAGATCCTTGAGAGTTTTCGCCCCGAAGAACGCTTTCCAATGATGAGCACTTT |
| | TAAAGTTCTGCTATGTGGCGCGGTATTATCCCGTATTGACGCCGGGCAAGAGCAACTCGGTCGCCGC |
| | ATACACTATTCTCAGAATGACTTGGTTGAGTATTCACCAGTCACAGAAAAGCATCTTACGGATGGCA |
| | TGACAGTAAGAGAATTATGCAGTGCTGCCATAACCATGAGTGATAACACTGCGGCCAACTTACTTCT |
| | GACAACGATTGGAGGACCGAAGGAGCTAACCGCTTTTTTGCACAACATGGGGGATCATGTAACTCGC |
| | CTTGATCGTTGGGAACCGGAGCTGAATGAAGCCATACCAAACGACGAGCGTGACACCACGATGCCTG |
| | TAGCAATGGCAACAACCTTGCGTAAACTATTAACTGGCGAACTACTTACTCTAGCTTCCCGGCAACA |
| | GTTGATAGACTGGATGGAGGCGGATAAAGTTGCAGGACCACTTCTGCGCTCGGCCCTTCCGGCTGGC |
| | TGGTTTATTGCTGATAAATCTGGAGCCGGTGAGCGTGGGTCTCGCGGTATCATTGCAGCACTGGGGC |
| | CAGATGGTAAGCCCTCCCGTATCGTAGTTATCTACACGACGGGGAGTCAGGCAACTATGGATGAACG |
| | AAATAGACAGATCGCTGAGATAGGTGCCTCACTGATTAAGCATTGGTAACCGATTCTAGGTGCATTG |
| | GCGCAGAAAAAAATGCCTGATGCGACGCTGCGCGTCTTATACTCCCACATATGCCAGATTCAGCAAC |
| | GGATACGGCTTCCCCAACTTGCCCACTTCCATACGTGTCCTCCTTACCAGAAATTTATCCTTAAGAT |
| | CGTTTAAACTCGACTCTGGCTCTATCGAATCTCCGTCGTTTCGAGCTTACGCGAACAGCCGTGGCGC |
| | TCATTTGCTCGTCGGGCATCGAATCTCGTCAGCTATCGTCAGCTTACCTTTTTGGCAGCGATCGCGG |
| | CTCCCGACATCTTGGACCATTAGCTCCACAGGTATCTTCTTCCCTCTAGTGGTCATAACAGCAGCTT |
| | CAGCTACCTCTCAATTCAAAAAACCCCTCAAGACCCGTTTAGAGGCCCCAAGGGGTTATGCTATCAA |
| | TCGTTGCGTTACACACACAAAAAACCAACACACATCCATCTTCGATGGATAGCGATTTTATTATCTA |
| | ACTGCTGATCGAGTGTAGCCAGATCTAGTAATCAATTACGGGGTCATTAGTTCATAGCCCATATATG |
| | GAGTTCCGCGTTACATAACTTACGGTAAATGGCCCGCCTGGCTGACCGCCCAACGACCCCCGCCCAT |
| | TGACGTCAATAATGACGTATGTTCCCATAGTAACGCCAATAGGGACTTTCCATTGACGTCAATGGGT |
| | GGAGTATTTACGGTAAACTGCCCACTTGGCAGTACATCAAGTGTATCATATGCCAAGTACGCCCCCT |
| | ATTGACGTCAATGACGGTAAATGGCCCGCCTGGCATTATGCCCAGTACATGACCTTATGGGACTTTC |
| | CTACTTGGCAGTACATCTACGTATTAGTCATCGCTATTACCATGCTGATGCGGTTTTGGCAGTACAT |
| | CAATGGGCGTGGATAGCGGTTTGACTCACGGGGATTTCCAAGTCTCCACCCCATTGACGTCAATGGG |
| | AGTTTGTTTTGGCACCAAAATCAACGGGACTTTCCAAAATGTCGTAACAACTCCGCCCCATTGACGC |
| | AAATGGGCGGTAGGCGTGTACGGTGGGAGGTCTATATAAGCAGAGCTGGTTTAGTGAACCGTCAGAT |
| | CAGATCTTTGTCGATCCTACCATCCACTCGACACACCCGCCAGCGGCCGCTAATACGACTCACTATA |
| | GGGAGAAGTACTGCCACCATGGGCAAGAAGCAAAATCGCAAGACGGGGAATTCCAAGACACAATCCG |
| | CTAGCCCACCACCTAAAGAGCGTTCTAGCTCCCCTGCTACTGAGCAGTCCTGGATGGAAAACGACTT |
| | CGATGAACTCCGGGAAGAGGGATTTAGGCGATCCAACTATTCAGAACTCCGCGAAGATATCCAGACA |
| | AAGGGGAAGGAAGTCGAGAATTTCGAGAAGAACCTCGAGGAGTGCATCACCCGTATCACAAACACTG |
| | AGAAATGTCTCAAAGAACTCATGGAACTTAAGACAAAAGCCAGGGAGCTTCGAGAGGAGTGTCGGAG |
| | TCTGAGATCCAGGTGTGACCAGCTCGAGGAGCGCGTGAGCGCGATGGAAGACGAGATGAACGAGATG |
| | AAAAGAGAGGGCAAATTCAGGGAGAAGCGCATTAAGAGGAACGAACAGAGTCTGCAGGAGATTTGGG |
| | ATTACGTCAAGAGGCCTAACCTGCGGTTGATCGGCGTCCCCGAGAGCGACGTAGAAAACGGGACTAA |
| | ACTGGAGAATACACTTCAAGACATCATTCAAGAAAATTTTCCAAACCTGGCTCGGCAAGCTAATGTG |
| | CAAATCCAAGAGATCCAACGCACACCCCAGCGGTATAGCTCTCGGCGTGCCACCCCTAGGCATATTA |
| | TCGTGCGCTTTACTAAGGTGGAGATGAAAGAGAAGATGCTGCGAGCCGCTCGGGAAAAGGGAAGGGT |
| | GACTTTGAAGGGCAAACCTATTCGGCTGACGGTTGACCTTAGCGCCGAGACACTCCAGGCACGCCGG |
| | GAATGGGGCCCCATCTTTAATATCCTGAAGGAGAAGAACTTCCAGCCACGAATCTCTTACCCTGCAA |
| | AGTTGAGTTTTATCTCCGAGGGTGAGATTAAGTATTTCATCGATAAACAGATGCTGCGAGACTTCGT |
| | GACAACTCGCCCAGCTCTCAAGGAACTGCTCAAAGAGGCTCTTAATATGGAGCGCAATAATAGATAT |
| | CAACCCTTGCAGAACCACGCAAAGATGccaaagaagaagcggaaggtcTGAGACAGCCGTCAGACCA |
| | TCAAGACTAGGAAGAAACTGCATCAACTAATGAGCAAAATCACCAGCTAACATCATAGTATACATGA |
| | CCGGCTCTAACTCACATATCACCATCCTTACACTTAACATTAACGGCCTCAACTCAGCTATCAAGCG |
| | CCATCGGCTGGCCAGCTGGATCAAATCACAGGATCCAAGCGTTTGTTGCATCCAAGAGACCCACCTG |
| | ACCTGTAGAGATACTCACCGCCTCAAGATCAAGGGATGGCGAAAGATTTATCAGGCGAACGGTAAGC |
| | AGAAGAAAGCCGGAGTCGCAATTCTGGTCTCAGACAAGACGGATTTCAAGCCCACCAAAATTAAGCG |
| | TGATAAGGAAGGTCACTATATTATGGTGAAAGGCAGCATACAGCAGGAAGAACTTACCATATTGAAC |
| | ATCTACGCGCCAAACACCGGCGCACCTCGCTTTATCAAACAGGTCCTGTCCGATCTGCAGCGAGATC |
| | TGGATTCTCATACGTTGATTATGGGTGATTTCAATACACCATTGAGCACCCTGGATCGCAGCACCAG |
| | GCAAAAGGTAAATAAAGACACGCAAGAGCTCAATAGCGCACTGCATCAGGCAGATCTCATTGATATT |
| | TATCGCACTCTTCATCCTAAGAGTACCGAGTACACATTCTTCAGCGCCCCACATCATACATACTCAA |
| | AGATCGATCATATCGTCGGCTCAAAGGCTCTGCTGTCAAAGTGCAAGCGCACAGAGATAATTACAAA |
| | TTACCTGTCAGATCATAGCGCGATCAAGCTCGAGCTGAGAATCAAGAACCTGACCCAGAGCCGGAGT |
| | ACCACTTGGAAGCTTAATAACCTGCTGCTCAACGATTATTGGGTCCACAATGAGATGAAGGCAGAGA |
| | TTAAAATGTTCTTCGAAACAAATGAGAATAAGGATACTACCTATCAAAACCTTTGGGATGCCTTTAA |
| | GGCCGTCTGCAGAGGCAAGTTCATCGCCCTCAACGCCTATAAAAGAAAACAAGAGAGATCTAAGATC |
| | GATACTCTCACCTCTCAGCTGAAGGAGTTGGAGAAACAGGAACAGACCCACTCCAAGGCGTCAAGAC |
| | GGCAGGAGATCACAAAGATTCGCGCCGAGTTGAAAGAGATCGAAACCCAAAAGACTCTTCAGAAAAT |
| | TAACGAGTCTCGTAGTTGGTTCTTCGAGCGGATTAATAAGATAGACAGACCTCTGGCACGACTGATT |
| | AAGAAGAAGCGCGAAAAGAACCAGATTGATACCATCAAGAACGACAAGGGCGACATCACTACTGACC |
| | CGACCGAGATCCAGACCACTATTCGGGAGTATTATAAGCATTTGTATGCTAACAAGCTTGAGAACCT |
| | GGAAGAGATGGACACTTTTCTGGATACCTATACTCTGCCACGGCTTAATCAAGAGGAAGTCGAGTCC |
| | CTCAACCGCCCAATTACAGGAAGCGAGATTGTGGCCATAATTAACTCCCTGCCGACAAAGAAATCTC |
| | CTGGTCCGGACGGGTTTACAGCTGAGTTTTATCAACGGTATATGGAAGAGCTTGTACCGTTTCTGCT |
| | CAAGCTCTTTCAGTCTATAGAAAAGGAAGGCATCTTGCCCAATTCCTTCTACGAAGCTTCTATAATA |
| | CTTATTCCCAAACCAGGACGCGATACCACAAAGAAGGAAAACTTCCGGCCCATTAGTCTCATGAATA |
| | TCGACGCTAAAATATTGAACAAGATTCTCGCCAACAGAATCCAACAACATATTAAGAAATTGATACA |
| | TCACGACCAGGTGGGGTTTATACCTGGCATGCAGGGCTGGTTTAACATCCGGAAGAGTATTAACGTC |
| | ATTCAACACATTAATAGAGCTAAGGATAAGAATCATATGATCATCTCTATAGACGCGGAAAAGGCAT |
| | TCGATAAGATTCAGCAGCCATTTATGCTCAAGACTCTGAACAAACTCGGCATCGACGGAACATATTT |
| | TAAGATTATTCGCGCAATTTACGATAAGCCGACTGCTAACATTATCCTTAACGGCCAAAAGCTCGAG |
| | GCCTTTCCGCTCAAGACTGGAACCCGCCAAGGCTGTCCCCTCTCCCCGCTTTTGTTTAATATTGTAC |
| | TCGAGGTGCTGGCTAGGGCTATTCGTCAAGAGAAAGAGATTAAAGGGATACAGCTCGGGAAGGAAGA |
| | GGTCAAGCTTTCCTTGTTCGCCGATGATATGATTGTGTACCTGGAGAATCCTATTGTGTCTGCTCAG |
| | AACCTTCTTAAACTTATTTCTAACTTTAGCAAGGTCAGCGGCTATAAGATTAACGTCCAGAAATCTC |
| | AGGCCTTTCTGTACACAAATAATCGACAGACCGAATCCCAGATAATGGGTGAGCTTCCGTTTGTCAT |
| | AGCCAGCAAAAGGATAAAGTATCTCGGAATCCAGCTGACACGAGACGTTAAAGATTTGTTTAAGGAA |
| | AATTACAAGCCTCTCCTGAAAGAGATTAAGGAAGATACTAATAAGTGGAAGAATATCCCCTGTTCAT |
| | GGGTTGGCAGAATCAACATAGTGAAGATGGCAATACTTCCTAAAGTGATATATCGCTTTAACGCCAT |
| | CCCAATTAAACTGCCTATGACCTTCTTTACGGAGCTCGAGAAAACAACCCTTAAATTTATATGGAAT |
| | CAAAAGAGAGCAAGAATAGCGAAGTCCATCTTGAGCCAGAAGAATAAGGCCGGTGGGATTACTTTGC |
| | CTGATTTTAAGTTGTATTATAAAGCCACAGTAACTAAGACAGCCTGGTATTGGTATCAGAATAGAGA |
| | CATCGACCAGTGGAATCGGACCGAACCATCAGAGATAATGCCCCACATCTATAATTACCTTATATTC |
| | GATAAGCCAGAAAAGAATAAACAGTGGGGCAAAGACAGCCTCTTCAACAAGTGGTGTTGGGAGAATT |
| | GGCTGGCCATATGCCGGAAACTCAAGCTCGACCCCTTTCTTACACCCTACACTAAAATCAACAGTAG |
| | GTGGATCAAGGACTTGAATGTCAAGCCAAAGACTATAAAGACACTGGAAGAGAATCTTGGGATCACA |
| | ATACAAGATATAGGCGTCGGCAAAGATTTTATGTCAAAGACGCCCAAGGCCATGGCCACTAAGGATA |
| | AGATTGATAAGTGGGACCTTATTAAGCTCAAAAGCTTCTGTACTGCCAAGGAGACCACGATCAGAGT |
| | TAATAGGCAGCCCACTACATGGGAAAAGATTTTCGCCACTTATTCATCAGATAAGGGGTTGATAAGC |
| | AGAATATATAACGAGCTGAAGCAGATCTACAAGAAGAAAACGAATAATCCCATCAAGAAGTGGGCAA |
| | AAGATATGAACAGGCATTTTAGCAAAGAGGATATCTACGCCGCGAAGAAGCATATGAAGAAGTGTAG |
| | TTCAAGCTTGGCCATTCGTGAGATGCAGATTAAGACGACCATGCGATACCACCTTACCCCAGTGAGG |
| | ATGGCAATTATCAAGAAATCTGGCAATAATAGATGTTGGCGGGGCTGTGGCGAGATTGGCACCCTGC |
| | TCCATTGCTGGTGGGATTGCAAGCTGGTGCAGCCGCTTTGGAAATCAGTCTGGCGCTTTCTGAGGGA |
| | CCTCGAGCTTGAGATTCCCTTCGATCCCGCAATTCCCTTGCTCGGAATCTATCCTAACGAATACAAG |
| | AGCTGTTGTTACAAGGATACGTGTACCCGGATGTTCATCGCGGCCTTGTTTACGATAGCTAAGACGT |
| | GGAATCAGCCTAAGTGCCCCACAATGATCGATTGGATCAAGAAAATGTGGCATATTTATACCATGGA |
| | GTATTACGCAGCAATTAAGAATGACGAATTTATTTCCTTCGTTGGGACCTGGATGAAGCTGGAGACT |
| | ATTATTCTGAGCAAGCTGTCTCAGGAGCAAAAGACAAAGCATAGAATCTTCTCTCTCATTGGTGGTA |
| | ACGACTACAAAGACGATGACGACAAGTAAAGCGCTTCTAGAAGTTGTCTCCTCCTGCACTGACTGAC |
| | TGATACAATCGATTTCTGGATCCGCAGGCCTAATCAACCTCTGGATTACAAAATTTGTGAAAGATTG |
| | ACTGGTATTCTTAACTATGTTGCTCCTTTTACGCTATGTGGATACGCTGCTTTAATGCCTTTGTATC |
| | ATGCTATTGCTTCCCGTATGGCTTTCATTTTCTCCTCCTTGTATAAATCCTGGTTGCTGTCTCTTTA |
| | TGAGGAGTTGTGGCCCGTTGTCAGGCAACGTGGCGTGGTGTGCACTGTGTTTGCTGACGCAACCCCC |
| | ACTGGTTGGGGCATTGCCACCACCTGTCAGCTCCTTTCCGGGACTTTCGCTTTCCCCCTCCCTATTG |
| | CCACGGCGGAACTCATCGCCGCCTGCCTTGCCCGCTGCTGGACAGGGGCTCGGCTGTTGGGCACTGA |
| | CAATTCCGTGGTGTTGTCGGGGAAGCTGACGTCCTTTCCATGGCTGCTCGCCTGTGTTGCCACCTGG |
| | ATTCTGCGCGGGACGTCCTTCTGCTACGTCCCTTCGGCCCTCAATCCAGCGGACCTTCCTTCCCGCt |
| | gagagacacaaaaaattccaacacactattgcaatgaaaataaatttcctttattagccagaagtca |
| | gatgctcaaggggcttcatgatgtccccataatttttggcagagggaaaaagatctcagtggtattt |
| | gtgagccagggcattggccttctgataggcagcctgcacctgaggagtgcggccgctttacttgtac |
| | agctcgtccatgccgagagtgatcccggcggcggtcacgaactccagcaggaccatgtgatcgcgct |
| | tctcgttggggtctttgctcagggcggactgggtgctcaggtagtggttgtcgggcagcagcacggg |
| | gccgtcgccgatgggggtgttctgctggtagtggtcggcgagctgcacgctgccgtcctcgatgttg |
| | tggcggatcttgaagttcaccttgatgccgttcttctgcttgtcggccatgatatagacgttgtggc |
| | tgttgtagttgtactccagcttgtgccccaggatgttgccgtcctccttgaagtcgatgcccttcag |
| | ctcgatgcggttcaccagggtgtcgccctcgaacttcacctcggcgcgggtcttgtagttgccgtcg |
| | tccttgaagaagatggtgcgctcctggacgtagccttcgggcatggcggacttgaagaagtcgtgct |
| | gcttcatgtggtcggggtagcggctgaagcactgcacgccgtaggtcagggtggtcacgagggtggg |
| | ccagggcacgggcagcttgccggtggtgcagatgaacttcagggtcagcttgccgtaggtggcatcg |
| | ccctcgccctcgccggacacgctgaacttgtggccgtttacgtcgccgtccagctcgaccaggatgg |
| | gcaccaccccggtgaacagctcctcgcccttgctcaccatggtggcgggatctgacggttcactaaa |
| | ccagctctgcttatatagacctcccaccgtacacgcctaccgcccatttgcgtcaatggggcggagt |
| | tgttacgacattttggaaagtcccgttgattttggtgccaaaacaaactcccattgacgtcaatggg |
| | gtggagacttggaaatccccgtgagtcaaaccgctatccacgcccattgatgtactgccaaaaccgc |
| | atcaccatggtaatagcgatgactaatacgtagatgtactgccaagtaggaaagtcccataaggtca |
| | tgtactgggcataatgccaggcgggccatttaccgtcattgacgtcaatagggggcgtacttggcat |
| | atgatacacttgatgtactgccaagtgggcagtttaccgtaaatactccacccattgacgtcaatgg |
| | aaagtccctattggcgttactatgggaacatacgtcattattgacgtcaatgggcgggggtcgttgg |
| | gcggtcagccaggcgggccatttaccgtaagttatgtaacgGGCCTGCTGCCGGCTCTGCGGCCTCT |
| | TCCGCGTCTTCGCCTTCGCCCTCAGACGAGTCGGATCTCCCTTTGGGCCGCCTCCCCGCCTGTCTAG |
| | CTTGACTGACTGAGATACAGCGTACCTTCAGCTCACAGACATGATAAGATACATTGATGAGTTTGGA |
| | CAAACCACAACTAGAATGCAGTGAAAAAAATGCTTTATTTGTGAAATTTGTGATGCTATTGCTTTAT |
| | TTGTAACCATTATAAGCTGCAATAAACAAGTTAACAACAACAATTGCATTCATTTTATGTTTCAGGT |
| | TCAGGGGGAGGTGTGGGAGGTTTTTTAAAGCAAGTAAAACCTCTACAAATGTGGTATTGGCCCATCT |
| | CTATCGGTATCGTAGCATAACCCCTTGGGGCCTCTAAACGGGTCTTGAGGGGTTTTTTGTGCCCCTC |
| | GGGCCGGATTGCTATCTACCGGCATTGGCGCAGAAAAAAATGCCTGATGCGACGCTGCGCGTCTTAT |
| | ACTCCCACATATGCCAGATTCAGCAACGGATACGGCTTCCCCAACTTGCCCACTTCCATACGTGTCC |
| | TCCTTACCAGAAATTTATCCTTAAGGTCGTCAGCTATCCTGCAGGCGATCTCTCGATTTCGATCAAG |
| | ACATTCCTTTAATGGTCTTTTCTGGACACCACTAGGGGTCAGAAGTAGTTCATCAAACTTTCTTCCC |
| | TCCCTAATCTCATTGGTTACCTTGGGCTATCGAAACTTAATTAAGCGATCTGCATCTCAATTAGTCA |
| | GCAACCATAGTCCCGCCCCTAACTCCGCCCATCCCGCCCCTAACTCCGCCCAGTTCCGCCCATTCTC |
| | CGCCCCATCGCTGACTAATTTTTTTTATTTATGCAGAGGCCGAGGCCGCCTCGGCCTCTGAGCTATT |
| | CCAGAAGTAGTGAGGAGGCTTTTTTGGAGGCCTAGGCTTTTGCAAAGGAGGTAGCCAACATGATTGA |
| | ACAAGATGGATTGCACGCAGGTTCTCCCGCCGCTTGGGTGGAGAGGCTATTCGGCTATGACTGGGCA |
| | CAACAGACAATCGGCTGCTCTGATGCCGCCGTGTTCCGGCTGTCAGCGCAGGGGCGCCCGGTTCTTT |
| | TTGTCAAGACCGACCTGTCCGGTGCCCTGAATGAACTCCAGGACGAGGCAGCGCGGCTATCGTGGCT |
| | GGCCACGACGGGCGTTCCTTGCGCAGCTGTGCTCGACGTTGTCACTGAAGCGGGAAGGGACTGGCTG |
| | CTATTGGGCGAAGTGCCGGGGCAGGATCTCCTGTCATCTCACCTTGCTCCTGCCGAGAAAGTATCCA |
| | TCATGGCTGATGCAATGCGGCGGCTGCATACGCTTGATCCGGCTACCTGCCCATTCGACCACCAAGC |
| | GAAACATCGCATCGAGCGAGCACGTACTCGGATGGAAGCCGGTCTTGTCGATCAGGATGATCTGGAC |
| | GAAGAGCATCAGGGGCTCGCGCCAGCCGAACTGTTCGCCAGGCTCAAGGCGCGGATGCCCGACGGCG |
| | AGGATCTCGTCGTGACCCACGGCGATGCCTGCTTGCCGAATATCATGGTGGAAAATGGCCGCTTTTC |
| | TGGATTCATCGACTGTGGCCGGCTGGGTGTGGCGGACCGCTATCAGGACATAGCGTTGGCTACCCGT |
| | GATATTGCTGAAGAGCTTGGCGGCGAATGGGCTGACCGCTTCCTCGTGCTTTACGGTATCGCCGCTC |
| | CCGATTCGCAGCGCATCGCCTTCTATCGCCTTCTTGACGAGTTCTTCTAGTATGTAAGCCCTGTGCC |
| | TTCTAGTTGCCAGCCATCTGTTGTTTGCCCCTCCCCCGTGCCTTCCTTGACCCTGGAAGGTGCCACT |
| | CCCACTGTCCTTTCCTAATAAAATGAGGAAATTGCATCGCATTGTCTGAGTAGGTGTCATTCTATTC |
| | TGGGGGGTGGGGTGGGGCAGGACAGCAAGGGGGAGGATTGGGAAGACAATAGCAGGCATGCTGGGGA |
| | TGCGGTGGGCTCTATGGTTAATTAACCAGTCAAGTCAGCTACTTGGCGAGATCGACTTGTCTGGGTT |
| | TCGACTACGCTCAGAATTGCGTCAGTCAAGTTCGATCTGGTCCTTGCTATTGCACCCGTTCTCCGAT |
| | TACGAGTTTCATTTAAATCATGTGAGCAAAAGGCCAGCAAAAGGCCAGGAACCGTAAAAAGGCCGCG |
| | TTGCTGGCGTTTTTCCATAGGCTCCGCCCCCCTGACGAGCATCACAAAAATCGACGCTCAAGTCAGA |
| | GGTGGCGAAACCCGACAGGACTATAAAGATACCAGGCGTTTCCCCCTGGAAGCTCCCTCGTGCGCTC |
| | TCCTGTTCCGACCCTGCCGCTTACCGGATACCTGTCCGCCTTTCTCCCTTCGGGAAGCGTGGCGCTT |
| | TCTCATAGCTCACGCTGTAGGTATCTCAGTTCGGTGTAGGTCGTTCGCTCCAAGCTGGGCTGTGTGC |
| | ACGAACCCCCCGTTCAGCCCGACCGCTGCGCCTTATCCGGTAACTATCGTCTTGAGTCCAACCCGGT |
| | AAGACACGACTTATCGCCACTGGCAGCAGCCACTGGTAACAGGATTAGCAGAGCGAGGTATGTAGGC |
| | GGTGCTACAGAGTTCTTGAAGTGGTGGCCTAACTACGGCTACACTAGAAGAACAGTATTTGGTATCT |
| | GCGCTCTGCTGAAGCCAGTTACCTTCGGAAAAAGAGTTGGTAGCTCTTGATCCGGCAAACAAACCAC |
| | CGCTGGTAGCGGTGGTTTTTTTGTTTGCAAGCAGCAGATTACGCGCAGAAAAAAAGGATCTCAAGAA |
| | GATCCTTTGATCTTTTCTACGGGGTCTGACGCTCAGTGGAACGAAAACTCACGTTAAGGGATTTTGG |
| | TCATGAGATTATCAAAAAGGATCTTCACCTAGATCCTTTTAAATTAAAAATGAAGTTTTAAATCAAT |
| | CTAAAGTATATATGAGTAAACTTGGTCTGACAGTTACCAATGCTTAATCAGTGAGGCACCTATCTCA |
| | GCGATCTGTCTATTTCGTTCATCCATAGTTGCATTTAAATTTCCGAACTCTCCAAGGCCCTCGTCGG |
| | AAAATCTTCAAAC (SEQ ID NO: 92) |
| >ret- | CCTCTCAGTGCGAGTCTCGACGATCCATATCGTTGCTTGGCAGTCAGCCAGTCGGAATCCAGCTTGG |
| 003- | GACCCAGGAAGTCCAATCGTCAGATATTGTACTCAAGCCTGGTCACGGCAGCGTACCGATCTGTTTA |
| line1- | AACCTAGATATTGATAGTCTGATCGGTCAACGTATAATCGAGTCCTAGCTTTTGCAAACATCTATCA |
| gfp- | AGAGACAGGATCAGCAGGAGGCTTTCGCATGAGTATTCAACATTTCCGTGTCGCCCTTATTCCCTTT |
| orf1-c- | TTTGCGGCATTTTGCCTTCCTGTTTTTGCTCACCCAGAAACGCTGGTGAAAGTAAAAGATGCTGAAG |
| linker_ | ATCAGTTGGGTGCGCGAGTGGGTTACATCGAACTGGATCTCAACAGCGGTAAGATCCTTGAGAGTTT |
| sv40-nls | TCGCCCCGAAGAACGCTTTCCAATGATGAGCACTTTTAAAGTTCTGCTATGTGGCGCGGTATTATCC |
| | CGTATTGACGCCGGGCAAGAGCAACTCGGTCGCCGCATACACTATTCTCAGAATGACTTGGTTGAGT |
| | ATTCACCAGTCACAGAAAAGCATCTTACGGATGGCATGACAGTAAGAGAATTATGCAGTGCTGCCAT |
| | AACCATGAGTGATAACACTGCGGCCAACTTACTTCTGACAACGATTGGAGGACCGAAGGAGCTAACC |
| | GCTTTTTTGCACAACATGGGGGATCATGTAACTCGCCTTGATCGTTGGGAACCGGAGCTGAATGAAG |
| | CCATACCAAACGACGAGCGTGACACCACGATGCCTGTAGCAATGGCAACAACCTTGCGTAAACTATT |
| | AACTGGCGAACTACTTACTCTAGCTTCCCGGCAACAGTTGATAGACTGGATGGAGGCGGATAAAGTT |
| | GCAGGACCACTTCTGCGCTCGGCCCTTCCGGCTGGCTGGTTTATTGCTGATAAATCTGGAGCCGGTG |
| | AGCGTGGGTCTCGCGGTATCATTGCAGCACTGGGGCCAGATGGTAAGCCCTCCCGTATCGTAGTTAT |
| | CTACACGACGGGGAGTCAGGCAACTATGGATGAACGAAATAGACAGATCGCTGAGATAGGTGCCTCA |
| | CTGATTAAGCATTGGTAACCGATTCTAGGTGCATTGGCGCAGAAAAAAATGCCTGATGCGACGCTGC |
| | GCGTCTTATACTCCCACATATGCCAGATTCAGCAACGGATACGGCTTCCCCAACTTGCCCACTTCCA |
| | TACGTGTCCTCCTTACCAGAAATTTATCCTTAAGATCGTTTAAACTCGACTCTGGCTCTATCGAATC |
| | TCCGTCGTTTCGAGCTTACGCGAACAGCCGTGGCGCTCATTTGCTCGTCGGGCATCGAATCTCGTCA |
| | GCTATCGTCAGCTTACCTTTTTGGCAGCGATCGCGGCTCCCGACATCTTGGACCATTAGCTCCACAG |
| | GTATCTTCTTCCCTCTAGTGGTCATAACAGCAGCTTCAGCTACCTCTCAATTCAAAAAACCCCTCAA |
| | GACCCGTTTAGAGGCCCCAAGGGGTTATGCTATCAATCGTTGCGTTACACACACAAAAAACCAACAC |
| | ACATCCATCTTCGATGGATAGCGATTTTATTATCTAACTGCTGATCGAGTGTAGCCAGATCTAGTAA |
| | TCAATTACGGGGTCATTAGTTCATAGCCCATATATGGAGTTCCGCGTTACATAACTTACGGTAAATG |
| | GCCCGCCTGGCTGACCGCCCAACGACCCCCGCCCATTGACGTCAATAATGACGTATGTTCCCATAGT |
| | AACGCCAATAGGGACTTTCCATTGACGTCAATGGGTGGAGTATTTACGGTAAACTGCCCACTTGGCA |
| | GTACATCAAGTGTATCATATGCCAAGTACGCCCCCTATTGACGTCAATGACGGTAAATGGCCCGCCT |
| | GGCATTATGCCCAGTACATGACCTTATGGGACTTTCCTACTTGGCAGTACATCTACGTATTAGTCAT |
| | CGCTATTACCATGCTGATGCGGTTTTGGCAGTACATCAATGGGCGTGGATAGCGGTTTGACTCACGG |
| | GGATTTCCAAGTCTCCACCCCATTGACGTCAATGGGAGTTTGTTTTGGCACCAAAATCAACGGGACT |
| | TTCCAAAATGTCGTAACAACTCCGCCCCATTGACGCAAATGGGCGGTAGGCGTGTACGGTGGGAGGT |
| | CTATATAAGCAGAGCTGGTTTAGTGAACCGTCAGATCAGATCTTTGTCGATCCTACCATCCACTCGA |
| | CACACCCGCCAGCGGCCGCTAATACGACTCACTATAGGGAGAAGTACTGCCACCATGGGCAAGAAGC |
| | AAAATCGCAAGACGGGGAATTCCAAGACACAATCCGCTAGCCCACCACCTAAAGAGCGTTCTAGCTC |
| | CCCTGCTACTGAGCAGTCCTGGATGGAAAACGACTTCGATGAACTCCGGGAAGAGGGATTTAGGCGA |
| | TCCAACTATTCAGAACTCCGCGAAGATATCCAGACAAAGGGGAAGGAAGTCGAGAATTTCGAGAAGA |
| | ACCTCGAGGAGTGCATCACCCGTATCACAAACACTGAGAAATGTCTCAAAGAACTCATGGAACTTAA |
| | GACAAAAGCCAGGGAGCTTCGAGAGGAGTGTCGGAGTCTGAGATCCAGGTGTGACCAGCTCGAGGAG |
| | CGCGTGAGCGCGATGGAAGACGAGATGAACGAGATGAAAAGAGAGGGCAAATTCAGGGAGAAGCGCA |
| | TTAAGAGGAACGAACAGAGTCTGCAGGAGATTTGGGATTACGTCAAGAGGCCTAACCTGCGGTTGAT |
| | CGGCGTCCCCGAGAGCGACGTAGAAAACGGGACTAAACTGGAGAATACACTTCAAGACATCATTCAA |
| | GAAAATTTTCCAAACCTGGCTCGGCAAGCTAATGTGCAAATCCAAGAGATCCAACGCACACCCCAGC |
| | GGTATAGCTCTCGGCGTGCCACCCCTAGGCATATTATCGTGCGCTTTACTAAGGTGGAGATGAAAGA |
| | GAAGATGCTGCGAGCCGCTCGGGAAAAGGGAAGGGTGACTTTGAAGGGCAAACCTATTCGGCTGACG |
| | GTTGACCTTAGCGCCGAGACACTCCAGGCACGCCGGGAATGGGGCCCCATCTTTAATATCCTGAAGG |
| | AGAAGAACTTCCAGCCACGAATCTCTTACCCTGCAAAGTTGAGTTTTATCTCCGAGGGTGAGATTAA |
| | GTATTTCATCGATAAACAGATGCTGCGAGACTTCGTGACAACTCGCCCAGCTCTCAAGGAACTGCTC |
| | AAAGAGGCTCTTAATATGGAGCGCAATAATAGATATCAACCCTTGCAGAACCACGCAAAGATGggcg |
| | gcggcagcccaaagaagaagcggaaggtcTGAGACAGCCGTCAGACCATCAAGACTAGGAAGAAACT |
| | GCATCAACTAATGAGCAAAATCACCAGCTAACATCATAGTATACATGACCGGCTCTAACTCACATAT |
| | CACCATCCTTACACTTAACATTAACGGCCTCAACTCAGCTATCAAGCGCCATCGGCTGGCCAGCTGG |
| | ATCAAATCACAGGATCCAAGCGTTTGTTGCATCCAAGAGACCCACCTGACCTGTAGAGATACTCACC |
| | GCCTCAAGATCAAGGGATGGCGAAAGATTTATCAGGCGAACGGTAAGCAGAAGAAAGCCGGAGTCGC |
| | AATTCTGGTCTCAGACAAGACGGATTTCAAGCCCACCAAAATTAAGCGTGATAAGGAAGGTCACTAT |
| | ATTATGGTGAAAGGCAGCATACAGCAGGAAGAACTTACCATATTGAACATCTACGCGCCAAACACCG |
| | GCGCACCTCGCTTTATCAAACAGGTCCTGTCCGATCTGCAGCGAGATCTGGATTCTCATACGTTGAT |
| | TATGGGTGATTTCAATACACCATTGAGCACCCTGGATCGCAGCACCAGGCAAAAGGTAAATAAAGAC |
| | ACGCAAGAGCTCAATAGCGCACTGCATCAGGCAGATCTCATTGATATTTATCGCACTCTTCATCCTA |
| | AGAGTACCGAGTACACATTCTTCAGCGCCCCACATCATACATACTCAAAGATCGATCATATCGTCGG |
| | CTCAAAGGCTCTGCTGTCAAAGTGCAAGCGCACAGAGATAATTACAAATTACCTGTCAGATCATAGC |
| | GCGATCAAGCTCGAGCTGAGAATCAAGAACCTGACCCAGAGCCGGAGTACCACTTGGAAGCTTAATA |
| | ACCTGCTGCTCAACGATTATTGGGTCCACAATGAGATGAAGGCAGAGATTAAAATGTTCTTCGAAAC |
| | AAATGAGAATAAGGATACTACCTATCAAAACCTTTGGGATGCCTTTAAGGCCGTCTGCAGAGGCAAG |
| | TTCATCGCCCTCAACGCCTATAAAAGAAAACAAGAGAGATCTAAGATCGATACTCTCACCTCTCAGC |
| | TGAAGGAGTTGGAGAAACAGGAACAGACCCACTCCAAGGCGTCAAGACGGCAGGAGATCACAAAGAT |
| | TCGCGCCGAGTTGAAAGAGATCGAAACCCAAAAGACTCTTCAGAAAATTAACGAGTCTCGTAGTTGG |
| | TTCTTCGAGCGGATTAATAAGATAGACAGACCTCTGGCACGACTGATTAAGAAGAAGCGCGAAAAGA |
| | ACCAGATTGATACCATCAAGAACGACAAGGGCGACATCACTACTGACCCGACCGAGATCCAGACCAC |
| | TATTCGGGAGTATTATAAGCATTTGTATGCTAACAAGCTTGAGAACCTGGAAGAGATGGACACTTTT |
| | CTGGATACCTATACTCTGCCACGGCTTAATCAAGAGGAAGTCGAGTCCCTCAACCGCCCAATTACAG |
| | GAAGCGAGATTGTGGCCATAATTAACTCCCTGCCGACAAAGAAATCTCCTGGTCCGGACGGGTTTAC |
| | AGCTGAGTTTTATCAACGGTATATGGAAGAGCTTGTACCGTTTCTGCTCAAGCTCTTTCAGTCTATA |
| | GAAAAGGAAGGCATCTTGCCCAATTCCTTCTACGAAGCTTCTATAATACTTATTCCCAAACCAGGAC |
| | GCGATACCACAAAGAAGGAAAACTTCCGGCCCATTAGTCTCATGAATATCGACGCTAAAATATTGAA |
| | CAAGATTCTCGCCAACAGAATCCAACAACATATTAAGAAATTGATACATCACGACCAGGTGGGGTTT |
| | ATACCTGGCATGCAGGGCTGGTTTAACATCCGGAAGAGTATTAACGTCATTCAACACATTAATAGAG |
| | CTAAGGATAAGAATCATATGATCATCTCTATAGACGCGGAAAAGGCATTCGATAAGATTCAGCAGCC |
| | ATTTATGCTCAAGACTCTGAACAAACTCGGCATCGACGGAACATATTTTAAGATTATTCGCGCAATT |
| | TACGATAAGCCGACTGCTAACATTATCCTTAACGGCCAAAAGCTCGAGGCCTTTCCGCTCAAGACTG |
| | GAACCCGCCAAGGCTGTCCCCTCTCCCCGCTTTTGTTTAATATTGTACTCGAGGTGCTGGCTAGGGC |
| | TATTCGTCAAGAGAAAGAGATTAAAGGGATACAGCTCGGGAAGGAAGAGGTCAAGCTTTCCTTGTTC |
| | GCCGATGATATGATTGTGTACCTGGAGAATCCTATTGTGTCTGCTCAGAACCTTCTTAAACTTATTT |
| | CTAACTTTAGCAAGGTCAGCGGCTATAAGATTAACGTCCAGAAATCTCAGGCCTTTCTGTACACAAA |
| | TAATCGACAGACCGAATCCCAGATAATGGGTGAGCTTCCGTTTGTCATAGCCAGCAAAAGGATAAAG |
| | TATCTCGGAATCCAGCTGACACGAGACGTTAAAGATTTGTTTAAGGAAAATTACAAGCCTCTCCTGA |
| | AAGAGATTAAGGAAGATACTAATAAGTGGAAGAATATCCCCTGTTCATGGGTTGGCAGAATCAACAT |
| | AGTGAAGATGGCAATACTTCCTAAAGTGATATATCGCTTTAACGCCATCCCAATTAAACTGCCTATG |
| | ACCTTCTTTACGGAGCTCGAGAAAACAACCCTTAAATTTATATGGAATCAAAAGAGAGCAAGAATAG |
| | CGAAGTCCATCTTGAGCCAGAAGAATAAGGCCGGTGGGATTACTTTGCCTGATTTTAAGTTGTATTA |
| | TAAAGCCACAGTAACTAAGACAGCCTGGTATTGGTATCAGAATAGAGACATCGACCAGTGGAATCGG |
| | ACCGAACCATCAGAGATAATGCCCCACATCTATAATTACCTTATATTCGATAAGCCAGAAAAGAATA |
| | AACAGTGGGGCAAAGACAGCCTCTTCAACAAGTGGTGTTGGGAGAATTGGCTGGCCATATGCCGGAA |
| | ACTCAAGCTCGACCCCTTTCTTACACCCTACACTAAAATCAACAGTAGGTGGATCAAGGACTTGAAT |
| | GTCAAGCCAAAGACTATAAAGACACTGGAAGAGAATCTTGGGATCACAATACAAGATATAGGCGTCG |
| | GCAAAGATTTTATGTCAAAGACGCCCAAGGCCATGGCCACTAAGGATAAGATTGATAAGTGGGACCT |
| | TATTAAGCTCAAAAGCTTCTGTACTGCCAAGGAGACCACGATCAGAGTTAATAGGCAGCCCACTACA |
| | TGGGAAAAGATTTTCGCCACTTATTCATCAGATAAGGGGTTGATAAGCAGAATATATAACGAGCTGA |
| | AGCAGATCTACAAGAAGAAAACGAATAATCCCATCAAGAAGTGGGCAAAAGATATGAACAGGCATTT |
| | TAGCAAAGAGGATATCTACGCCGCGAAGAAGCATATGAAGAAGTGTAGTTCAAGCTTGGCCATTCGT |
| | GAGATGCAGATTAAGACGACCATGCGATACCACCTTACCCCAGTGAGGATGGCAATTATCAAGAAAT |
| | CTGGCAATAATAGATGTTGGCGGGGCTGTGGCGAGATTGGCACCCTGCTCCATTGCTGGTGGGATTG |
| | CAAGCTGGTGCAGCCGCTTTGGAAATCAGTCTGGCGCTTTCTGAGGGACCTCGAGCTTGAGATTCCC |
| | TTCGATCCCGCAATTCCCTTGCTCGGAATCTATCCTAACGAATACAAGAGCTGTTGTTACAAGGATA |
| | CGTGTACCCGGATGTTCATCGCGGCCTTGTTTACGATAGCTAAGACGTGGAATCAGCCTAAGTGCCC |
| | CACAATGATCGATTGGATCAAGAAAATGTGGCATATTTATACCATGGAGTATTACGCAGCAATTAAG |
| | AATGACGAATTTATTTCCTTCGTTGGGACCTGGATGAAGCTGGAGACTATTATTCTGAGCAAGCTGT |
| | CTCAGGAGCAAAAGACAAAGCATAGAATCTTCTCTCTCATTGGTGGTAACGACTACAAAGACGATGA |
| | CGACAAGTAAAGCGCTTCTAGAAGTTGTCTCCTCCTGCACTGACTGACTGATACAATCGATTTCTGG |
| | ATCCGCAGGCCTAATCAACCTCTGGATTACAAAATTTGTGAAAGATTGACTGGTATTCTTAACTATG |
| | TTGCTCCTTTTACGCTATGTGGATACGCTGCTTTAATGCCTTTGTATCATGCTATTGCTTCCCGTAT |
| | GGCTTTCATTTTCTCCTCCTTGTATAAATCCTGGTTGCTGTCTCTTTATGAGGAGTTGTGGCCCGTT |
| | GTCAGGCAACGTGGCGTGGTGTGCACTGTGTTTGCTGACGCAACCCCCACTGGTTGGGGCATTGCCA |
| | CCACCTGTCAGCTCCTTTCCGGGACTTTCGCTTTCCCCCTCCCTATTGCCACGGCGGAACTCATCGC |
| | CGCCTGCCTTGCCCGCTGCTGGACAGGGGCTCGGCTGTTGGGCACTGACAATTCCGTGGTGTTGTCG |
| | GGGAAGCTGACGTCCTTTCCATGGCTGCTCGCCTGTGTTGCCACCTGGATTCTGCGCGGGACGTCCT |
| | TCTGCTACGTCCCTTCGGCCCTCAATCCAGCGGACCTTCCTTCCCGCtgagagacacaaaaaattcc |
| | aacacactattgcaatgaaaataaatttcctttattagccagaagtcagatgctcaaggggcttcat |
| | gatgtccccataatttttggcagagggaaaaagatctcagtggtatttgtgagccagggcattggcc |
| | ttctgataggcagcctgcacctgaggagtgcggccgctttacttgtacagctcgtccatgccgagag |
| | tgatcccggcggcggtcacgaactccagcaggaccatgtgatcgcgcttctcgttggggtctttgct |
| | cagggcggactgggtgctcaggtagtggttgtcgggcagcagcacggggccgtcgccgatgggggtg |
| | ttctgctggtagtggtcggcgagctgcacgctgccgtcctcgatgttgtggcggatcttgaagttca |
| | ccttgatgccgttcttctgcttgtcggccatgatatagacgttgtggctgttgtagttgtactccag |
| | cttgtgccccaggatgttgccgtcctccttgaagtcgatgcccttcagctcgatgcggttcaccagg |
| | gtgtcgccctcgaacttcacctcggcgcgggtcttgtagttgccgtcgtccttgaagaagatggtgc |
| | gctcctggacgtagccttcgggcatggcggacttgaagaagtcgtgctgcttcatgtggtcggggta |
| | gcggctgaagcactgcacgccgtaggtcagggtggtcacgagggtgggccagggcacgggcagcttg |
| | ccggtggtgcagatgaacttcagggtcagcttgccgtaggtggcatcgccctcgccctcgccggaca |
| | cgctgaacttgtggccgtttacgtcgccgtccagctcgaccaggatgggcaccaccccggtgaacag |
| | ctcctcgcccttgctcaccatggtggcgggatctgacggttcactaaaccagctctgcttatataga |
| | cctcccaccgtacacgcctaccgcccatttgcgtcaatggggcggagttgttacgacattttggaaa |
| | gtcccgttgattttggtgccaaaacaaactcccattgacgtcaatggggtggagacttggaaatccc |
| | cgtgagtcaaaccgctatccacgcccattgatgtactgccaaaaccgcatcaccatggtaatagcga |
| | tgactaatacgtagatgtactgccaagtaggaaagtcccataaggtcatgtactgggcataatgcca |
| | ggcgggccatttaccgtcattgacgtcaatagggggcgtacttggcatatgatacacttgatgtact |
| | gccaagtgggcagtttaccgtaaatactccacccattgacgtcaatggaaagtccctattggcgtta |
| | ctatgggaacatacgtcattattgacgtcaatgggcgggggtcgttgggcggtcagccaggcgggcc |
| | atttaccgtaagttatgtaacgGGCCTGCTGCCGGCTCTGCGGCCTCTTCCGCGTCTTCGCCTTCGC |
| | CCTCAGACGAGTCGGATCTCCCTTTGGGCCGCCTCCCCGCCTGTCTAGCTTGACTGACTGAGATACA |
| | GCGTACCTTCAGCTCACAGACATGATAAGATACATTGATGAGTTTGGACAAACCACAACTAGAATGC |
| | AGTGAAAAAAATGCTTTATTTGTGAAATTTGTGATGCTATTGCTTTATTTGTAACCATTATAAGCTG |
| | CAATAAACAAGTTAACAACAACAATTGCATTCATTTTATGTTTCAGGTTCAGGGGGAGGTGTGGGAG |
| | GTTTTTTAAAGCAAGTAAAACCTCTACAAATGTGGTATTGGCCCATCTCTATCGGTATCGTAGCATA |
| | ACCCCTTGGGGCCTCTAAACGGGTCTTGAGGGGTTTTTTGTGCCCCTCGGGCCGGATTGCTATCTAC |
| | CGGCATTGGCGCAGAAAAAAATGCCTGATGCGACGCTGCGCGTCTTATACTCCCACATATGCCAGAT |
| | TCAGCAACGGATACGGCTTCCCCAACTTGCCCACTTCCATACGTGTCCTCCTTACCAGAAATTTATC |
| | CTTAAGGTCGTCAGCTATCCTGCAGGCGATCTCTCGATTTCGATCAAGACATTCCTTTAATGGTCTT |
| | TTCTGGACACCACTAGGGGTCAGAAGTAGTTCATCAAACTTTCTTCCCTCCCTAATCTCATTGGTTA |
| | CCTTGGGCTATCGAAACTTAATTAAGCGATCTGCATCTCAATTAGTCAGCAACCATAGTCCCGCCCC |
| | TAACTCCGCCCATCCCGCCCCTAACTCCGCCCAGTTCCGCCCATTCTCCGCCCCATCGCTGACTAAT |
| | TTTTTTTATTTATGCAGAGGCCGAGGCCGCCTCGGCCTCTGAGCTATTCCAGAAGTAGTGAGGAGGC |
| | TTTTTTGGAGGCCTAGGCTTTTGCAAAGGAGGTAGCCAACATGATTGAACAAGATGGATTGCACGCA |
| | GGTTCTCCCGCCGCTTGGGTGGAGAGGCTATTCGGCTATGACTGGGCACAACAGACAATCGGCTGCT |
| | CTGATGCCGCCGTGTTCCGGCTGTCAGCGCAGGGGCGCCCGGTTCTTTTTGTCAAGACCGACCTGTC |
| | CGGTGCCCTGAATGAACTCCAGGACGAGGCAGCGCGGCTATCGTGGCTGGCCACGACGGGCGTTCCT |
| | TGCGCAGCTGTGCTCGACGTTGTCACTGAAGCGGGAAGGGACTGGCTGCTATTGGGCGAAGTGCCGG |
| | GGCAGGATCTCCTGTCATCTCACCTTGCTCCTGCCGAGAAAGTATCCATCATGGCTGATGCAATGCG |
| | GCGGCTGCATACGCTTGATCCGGCTACCTGCCCATTCGACCACCAAGCGAAACATCGCATCGAGCGA |
| | GCACGTACTCGGATGGAAGCCGGTCTTGTCGATCAGGATGATCTGGACGAAGAGCATCAGGGGCTCG |
| | CGCCAGCCGAACTGTTCGCCAGGCTCAAGGCGCGGATGCCCGACGGCGAGGATCTCGTCGTGACCCA |
| | CGGCGATGCCTGCTTGCCGAATATCATGGTGGAAAATGGCCGCTTTTCTGGATTCATCGACTGTGGC |
| | CGGCTGGGTGTGGCGGACCGCTATCAGGACATAGCGTTGGCTACCCGTGATATTGCTGAAGAGCTTG |
| | GCGGCGAATGGGCTGACCGCTTCCTCGTGCTTTACGGTATCGCCGCTCCCGATTCGCAGCGCATCGC |
| | CTTCTATCGCCTTCTTGACGAGTTCTTCTAGTATGTAAGCCCTGTGCCTTCTAGTTGCCAGCCATCT |
| | GTTGTTTGCCCCTCCCCCGTGCCTTCCTTGACCCTGGAAGGTGCCACTCCCACTGTCCTTTCCTAAT |
| | AAAATGAGGAAATTGCATCGCATTGTCTGAGTAGGTGTCATTCTATTCTGGGGGGTGGGGTGGGGCA |
| | GGACAGCAAGGGGGAGGATTGGGAAGACAATAGCAGGCATGCTGGGGATGCGGTGGGCTCTATGGTT |
| | AATTAACCAGTCAAGTCAGCTACTTGGCGAGATCGACTTGTCTGGGTTTCGACTACGCTCAGAATTG |
| | CGTCAGTCAAGTTCGATCTGGTCCTTGCTATTGCACCCGTTCTCCGATTACGAGTTTCATTTAAATC |
| | ATGTGAGCAAAAGGCCAGCAAAAGGCCAGGAACCGTAAAAAGGCCGCGTTGCTGGCGTTTTTCCATA |
| | GGCTCCGCCCCCCTGACGAGCATCACAAAAATCGACGCTCAAGTCAGAGGTGGCGAAACCCGACAGG |
| | ACTATAAAGATACCAGGCGTTTCCCCCTGGAAGCTCCCTCGTGCGCTCTCCTGTTCCGACCCTGCCG |
| | CTTACCGGATACCTGTCCGCCTTTCTCCCTTCGGGAAGCGTGGCGCTTTCTCATAGCTCACGCTGTA |
| | GGTATCTCAGTTCGGTGTAGGTCGTTCGCTCCAAGCTGGGCTGTGTGCACGAACCCCCCGTTCAGCC |
| | CGACCGCTGCGCCTTATCCGGTAACTATCGTCTTGAGTCCAACCCGGTAAGACACGACTTATCGCCA |
| | CTGGCAGCAGCCACTGGTAACAGGATTAGCAGAGCGAGGTATGTAGGCGGTGCTACAGAGTTCTTGA |
| | AGTGGTGGCCTAACTACGGCTACACTAGAAGAACAGTATTTGGTATCTGCGCTCTGCTGAAGCCAGT |
| | TACCTTCGGAAAAAGAGTTGGTAGCTCTTGATCCGGCAAACAAACCACCGCTGGTAGCGGTGGTTTT |
| | TTTGTTTGCAAGCAGCAGATTACGCGCAGAAAAAAAGGATCTCAAGAAGATCCTTTGATCTTTTCTA |
| | CGGGGTCTGACGCTCAGTGGAACGAAAACTCACGTTAAGGGATTTTGGTCATGAGATTATCAAAAAG |
| | GATCTTCACCTAGATCCTTTTAAATTAAAAATGAAGTTTTAAATCAATCTAAAGTATATATGAGTAA |
| | ACTTGGTCTGACAGTTACCAATGCTTAATCAGTGAGGCACCTATCTCAGCGATCTGTCTATTTCGTT |
| | CATCCATAGTTGCATTTAAATTTCCGAACTCTCCAAGGCCCTCGTCGGAAAATCTTCAAACCTTTCG |
| | TCCGATCCATCTTGCAGGCTACCTCTCGAACGAACTATCGCAAGTCTCTTGGCCGGCCTTGCGCCTT |
| | GGCTATTGCTTGGCAGCGCCTATCGCCAGGTATTACTCCAATCCCGAATATCCGAGATCGGGATCAC |
| | CCGAGAGAAGTTCAACCTACATCCTCAATCCCGATCTATCCGAGATCCGAGGAATATCGAAATCGGG |
| | GCGCGCCTGGTGTACCGAGAACGAT (SEQ ID NO: 93) |
| >RET- | TCGAAATCGGGGCGCGCCTGGTGTACCGAGAACGATCCTCTCAGTGCGAGTCTCGACGATCCATATC |
| 004 | GTTGCTTGGCAGTCAGCCAGTCGGAATCCAGCTTGGGACCCAGGAAGTCCAATCGTCAGATATTGTA |
| LINE1- | CTCAAGCCTGGTCACGGCAGCGTACCGATCTGTTTAAACCTAGATATTGATAGTCTGATCGGTCAAC |
| GFP | GTATAATCGAGTCCTAGCTTTTGCAAACATCTATCAAGAGACAGGATCAGCAGGAGGCTTTCGCATG |
| ORF1-C | AGTATTCAACATTTCCGTGTCGCCCTTATTCCCTTTTTTGCGGCATTTTGCCTTCCTGTTTTTGCTC |
| Nucleo- | ACCCAGAAACGCTGGTGAAAGTAAAAGATGCTGAAGATCAGTTGGGTGCGCGAGTGGGTTACATCGA |
| plasmin- | ACTGGATCTCAACAGCGGTAAGATCCTTGAGAGTTTTCGCCCCGAAGAACGCTTTCCAATGATGAGC |
| NLS | ACTTTTAAAGTTCTGCTATGTGGCGCGGTATTATCCCGTATTGACGCCGGGCAAGAGCAACTCGGTC |
| | GCCGCATACACTATTCTCAGAATGACTTGGTTGAGTATTCACCAGTCACAGAAAAGCATCTTACGGA |
| | TGGCATGACAGTAAGAGAATTATGCAGTGCTGCCATAACCATGAGTGATAACACTGCGGCCAACTTA |
| | CTTCTGACAACGATTGGAGGACCGAAGGAGCTAACCGCTTTTTTGCACAACATGGGGGATCATGTAA |
| | CTCGCCTTGATCGTTGGGAACCGGAGCTGAATGAAGCCATACCAAACGACGAGCGTGACACCACGAT |
| | GCCTGTAGCAATGGCAACAACCTTGCGTAAACTATTAACTGGCGAACTACTTACTCTAGCTTCCCGG |
| | CAACAGTTGATAGACTGGATGGAGGCGGATAAAGTTGCAGGACCACTTCTGCGCTCGGCCCTTCCGG |
| | CTGGCTGGTTTATTGCTGATAAATCTGGAGCCGGTGAGCGTGGGTCTCGCGGTATCATTGCAGCACT |
| | GGGGCCAGATGGTAAGCCCTCCCGTATCGTAGTTATCTACACGACGGGGAGTCAGGCAACTATGGAT |
| | GAACGAAATAGACAGATCGCTGAGATAGGTGCCTCACTGATTAAGCATTGGTAACCGATTCTAGGTG |
| | CATTGGCGCAGAAAAAAATGCCTGATGCGACGCTGCGCGTCTTATACTCCCACATATGCCAGATTCA |
| | GCAACGGATACGGCTTCCCCAACTTGCCCACTTCCATACGTGTCCTCCTTACCAGAAATTTATCCTT |
| | AAGATCGTTTAAACTCGACTCTGGCTCTATCGAATCTCCGTCGTTTCGAGCTTACGCGAACAGCCGT |
| | GGCGCTCATTTGCTCGTCGGGCATCGAATCTCGTCAGCTATCGTCAGCTTACCTTTTTGGCAGCGAT |
| | CGCGGCTCCCGACATCTTGGACCATTAGCTCCACAGGTATCTTCTTCCCTCTAGTGGTCATAACAGC |
| | AGCTTCAGCTACCTCTCAATTCAAAAAACCCCTCAAGACCCGTTTAGAGGCCCCAAGGGGTTATGCT |
| | ATCAATCGTTGCGTTACACACACAAAAAACCAACACACATCCATCTTCGATGGATAGCGATTTTATT |
| | ATCTAACTGCTGATCGAGTGTAGCCAGATCTAGTAATCAATTACGGGGTCATTAGTTCATAGCCCAT |
| | ATATGGAGTTCCGCGTTACATAACTTACGGTAAATGGCCCGCCTGGCTGACCGCCCAACGACCCCCG |
| | CCCATTGACGTCAATAATGACGTATGTTCCCATAGTAACGCCAATAGGGACTTTCCATTGACGTCAA |
| | TGGGTGGAGTATTTACGGTAAACTGCCCACTTGGCAGTACATCAAGTGTATCATATGCCAAGTACGC |
| | CCCCTATTGACGTCAATGACGGTAAATGGCCCGCCTGGCATTATGCCCAGTACATGACCTTATGGGA |
| | CTTTCCTACTTGGCAGTACATCTACGTATTAGTCATCGCTATTACCATGCTGATGCGGTTTTGGCAG |
| | TACATCAATGGGCGTGGATAGCGGTTTGACTCACGGGGATTTCCAAGTCTCCACCCCATTGACGTCA |
| | ATGGGAGTTTGTTTTGGCACCAAAATCAACGGGACTTTCCAAAATGTCGTAACAACTCCGCCCCATT |
| | GACGCAAATGGGCGGTAGGCGTGTACGGTGGGAGGTCTATATAAGCAGAGCTGGTTTAGTGAACCGT |
| | CAGATCAGATCTTTGTCGATCCTACCATCCACTCGACACACCCGCCAGCGGCCGCTAATACGACTCA |
| | CTATAGGGAGAAGTACTGCCACCATGGGCAAGAAGCAAAATCGCAAGACGGGGAATTCCAAGACACA |
| | ATCCGCTAGCCCACCACCTAAAGAGCGTTCTAGCTCCCCTGCTACTGAGCAGTCCTGGATGGAAAAC |
| | GACTTCGATGAACTCCGGGAAGAGGGATTTAGGCGATCCAACTATTCAGAACTCCGCGAAGATATCC |
| | AGACAAAGGGGAAGGAAGTCGAGAATTTCGAGAAGAACCTCGAGGAGTGCATCACCCGTATCACAAA |
| | CACTGAGAAATGTCTCAAAGAACTCATGGAACTTAAGACAAAAGCCAGGGAGCTTCGAGAGGAGTGT |
| | CGGAGTCTGAGATCCAGGTGTGACCAGCTCGAGGAGCGCGTGAGCGCGATGGAAGACGAGATGAACG |
| | AGATGAAAAGAGAGGGCAAATTCAGGGAGAAGCGCATTAAGAGGAACGAACAGAGTCTGCAGGAGAT |
| | TTGGGATTACGTCAAGAGGCCTAACCTGCGGTTGATCGGCGTCCCCGAGAGCGACGTAGAAAACGGG |
| | ACTAAACTGGAGAATACACTTCAAGACATCATTCAAGAAAATTTTCCAAACCTGGCTCGGCAAGCTA |
| | ATGTGCAAATCCAAGAGATCCAACGCACACCCCAGCGGTATAGCTCTCGGCGTGCCACCCCTAGGCA |
| | TATTATCGTGCGCTTTACTAAGGTGGAGATGAAAGAGAAGATGCTGCGAGCCGCTCGGGAAAAGGGA |
| | AGGGTGACTTTGAAGGGCAAACCTATTCGGCTGACGGTTGACCTTAGCGCCGAGACACTCCAGGCAC |
| | GCCGGGAATGGGGCCCCATCTTTAATATCCTGAAGGAGAAGAACTTCCAGCCACGAATCTCTTACCC |
| | TGCAAAGTTGAGTTTTATCTCCGAGGGTGAGATTAAGTATTTCATCGATAAACAGATGCTGCGAGAC |
| | TTCGTGACAACTCGCCCAGCTCTCAAGGAACTGCTCAAAGAGGCTCTTAATATGGAGCGCAATAATA |
| | GATATCAACCCTTGCAGAACCACGCAAAGATGaaaaggccggcggccacgaaaaaggccggccaggc |
| | aaaaaagaaaaagTGAGACAGCCGTCAGACCATCAAGACTAGGAAGAAACTGCATCAACTAATGAGC |
| | AAAATCACCAGCTAACATCATAGTATACATGACCGGCTCTAACTCACATATCACCATCCTTACACTT |
| | AACATTAACGGCCTCAACTCAGCTATCAAGCGCCATCGGCTGGCCAGCTGGATCAAATCACAGGATC |
| | CAAGCGTTTGTTGCATCCAAGAGACCCACCTGACCTGTAGAGATACTCACCGCCTCAAGATCAAGGG |
| | ATGGCGAAAGATTTATCAGGCGAACGGTAAGCAGAAGAAAGCCGGAGTCGCAATTCTGGTCTCAGAC |
| | AAGACGGATTTCAAGCCCACCAAAATTAAGCGTGATAAGGAAGGTCACTATATTATGGTGAAAGGCA |
| | GCATACAGCAGGAAGAACTTACCATATTGAACATCTACGCGCCAAACACCGGCGCACCTCGCTTTAT |
| | CAAACAGGTCCTGTCCGATCTGCAGCGAGATCTGGATTCTCATACGTTGATTATGGGTGATTTCAAT |
| | ACACCATTGAGCACCCTGGATCGCAGCACCAGGCAAAAGGTAAATAAAGACACGCAAGAGCTCAATA |
| | GCGCACTGCATCAGGCAGATCTCATTGATATTTATCGCACTCTTCATCCTAAGAGTACCGAGTACAC |
| | ATTCTTCAGCGCCCCACATCATACATACTCAAAGATCGATCATATCGTCGGCTCAAAGGCTCTGCTG |
| | TCAAAGTGCAAGCGCACAGAGATAATTACAAATTACCTGTCAGATCATAGCGCGATCAAGCTCGAGC |
| | TGAGAATCAAGAACCTGACCCAGAGCCGGAGTACCACTTGGAAGCTTAATAACCTGCTGCTCAACGA |
| | TTATTGGGTCCACAATGAGATGAAGGCAGAGATTAAAATGTTCTTCGAAACAAATGAGAATAAGGAT |
| | ACTACCTATCAAAACCTTTGGGATGCCTTTAAGGCCGTCTGCAGAGGCAAGTTCATCGCCCTCAACG |
| | CCTATAAAAGAAAACAAGAGAGATCTAAGATCGATACTCTCACCTCTCAGCTGAAGGAGTTGGAGAA |
| | ACAGGAACAGACCCACTCCAAGGCGTCAAGACGGCAGGAGATCACAAAGATTCGCGCCGAGTTGAAA |
| | GAGATCGAAACCCAAAAGACTCTTCAGAAAATTAACGAGTCTCGTAGTTGGTTCTTCGAGCGGATTA |
| | ATAAGATAGACAGACCTCTGGCACGACTGATTAAGAAGAAGCGCGAAAAGAACCAGATTGATACCAT |
| | CAAGAACGACAAGGGCGACATCACTACTGACCCGACCGAGATCCAGACCACTATTCGGGAGTATTAT |
| | AAGCATTTGTATGCTAACAAGCTTGAGAACCTGGAAGAGATGGACACTTTTCTGGATACCTATACTC |
| | TGCCACGGCTTAATCAAGAGGAAGTCGAGTCCCTCAACCGCCCAATTACAGGAAGCGAGATTGTGGC |
| | CATAATTAACTCCCTGCCGACAAAGAAATCTCCTGGTCCGGACGGGTTTACAGCTGAGTTTTATCAA |
| | CGGTATATGGAAGAGCTTGTACCGTTTCTGCTCAAGCTCTTTCAGTCTATAGAAAAGGAAGGCATCT |
| | TGCCCAATTCCTTCTACGAAGCTTCTATAATACTTATTCCCAAACCAGGACGCGATACCACAAAGAA |
| | GGAAAACTTCCGGCCCATTAGTCTCATGAATATCGACGCTAAAATATTGAACAAGATTCTCGCCAAC |
| | AGAATCCAACAACATATTAAGAAATTGATACATCACGACCAGGTGGGGTTTATACCTGGCATGCAGG |
| | GCTGGTTTAACATCCGGAAGAGTATTAACGTCATTCAACACATTAATAGAGCTAAGGATAAGAATCA |
| | TATGATCATCTCTATAGACGCGGAAAAGGCATTCGATAAGATTCAGCAGCCATTTATGCTCAAGACT |
| | CTGAACAAACTCGGCATCGACGGAACATATTTTAAGATTATTCGCGCAATTTACGATAAGCCGACTG |
| | CTAACATTATCCTTAACGGCCAAAAGCTCGAGGCCTTTCCGCTCAAGACTGGAACCCGCCAAGGCTG |
| | TCCCCTCTCCCCGCTTTTGTTTAATATTGTACTCGAGGTGCTGGCTAGGGCTATTCGTCAAGAGAAA |
| | GAGATTAAAGGGATACAGCTCGGGAAGGAAGAGGTCAAGCTTTCCTTGTTCGCCGATGATATGATTG |
| | TGTACCTGGAGAATCCTATTGTGTCTGCTCAGAACCTTCTTAAACTTATTTCTAACTTTAGCAAGGT |
| | CAGCGGCTATAAGATTAACGTCCAGAAATCTCAGGCCTTTCTGTACACAAATAATCGACAGACCGAA |
| | TCCCAGATAATGGGTGAGCTTCCGTTTGTCATAGCCAGCAAAAGGATAAAGTATCTCGGAATCCAGC |
| | TGACACGAGACGTTAAAGATTTGTTTAAGGAAAATTACAAGCCTCTCCTGAAAGAGATTAAGGAAGA |
| | TACTAATAAGTGGAAGAATATCCCCTGTTCATGGGTTGGCAGAATCAACATAGTGAAGATGGCAATA |
| | CTTCCTAAAGTGATATATCGCTTTAACGCCATCCCAATTAAACTGCCTATGACCTTCTTTACGGAGC |
| | TCGAGAAAACAACCCTTAAATTTATATGGAATCAAAAGAGAGCAAGAATAGCGAAGTCCATCTTGAG |
| | CCAGAAGAATAAGGCCGGTGGGATTACTTTGCCTGATTTTAAGTTGTATTATAAAGCCACAGTAACT |
| | AAGACAGCCTGGTATTGGTATCAGAATAGAGACATCGACCAGTGGAATCGGACCGAACCATCAGAGA |
| | TAATGCCCCACATCTATAATTACCTTATATTCGATAAGCCAGAAAAGAATAAACAGTGGGGCAAAGA |
| | CAGCCTCTTCAACAAGTGGTGTTGGGAGAATTGGCTGGCCATATGCCGGAAACTCAAGCTCGACCCC |
| | TTTCTTACACCCTACACTAAAATCAACAGTAGGTGGATCAAGGACTTGAATGTCAAGCCAAAGACTA |
| | TAAAGACACTGGAAGAGAATCTTGGGATCACAATACAAGATATAGGCGTCGGCAAAGATTTTATGTC |
| | AAAGACGCCCAAGGCCATGGCCACTAAGGATAAGATTGATAAGTGGGACCTTATTAAGCTCAAAAGC |
| | TTCTGTACTGCCAAGGAGACCACGATCAGAGTTAATAGGCAGCCCACTACATGGGAAAAGATTTTCG |
| | CCACTTATTCATCAGATAAGGGGTTGATAAGCAGAATATATAACGAGCTGAAGCAGATCTACAAGAA |
| | GAAAACGAATAATCCCATCAAGAAGTGGGCAAAAGATATGAACAGGCATTTTAGCAAAGAGGATATC |
| | TACGCCGCGAAGAAGCATATGAAGAAGTGTAGTTCAAGCTTGGCCATTCGTGAGATGCAGATTAAGA |
| | CGACCATGCGATACCACCTTACCCCAGTGAGGATGGCAATTATCAAGAAATCTGGCAATAATAGATG |
| | TTGGCGGGGCTGTGGCGAGATTGGCACCCTGCTCCATTGCTGGTGGGATTGCAAGCTGGTGCAGCCG |
| | CTTTGGAAATCAGTCTGGCGCTTTCTGAGGGACCTCGAGCTTGAGATTCCCTTCGATCCCGCAATTC |
| | CCTTGCTCGGAATCTATCCTAACGAATACAAGAGCTGTTGTTACAAGGATACGTGTACCCGGATGTT |
| | CATCGCGGCCTTGTTTACGATAGCTAAGACGTGGAATCAGCCTAAGTGCCCCACAATGATCGATTGG |
| | ATCAAGAAAATGTGGCATATTTATACCATGGAGTATTACGCAGCAATTAAGAATGACGAATTTATTT |
| | CCTTCGTTGGGACCTGGATGAAGCTGGAGACTATTATTCTGAGCAAGCTGTCTCAGGAGCAAAAGAC |
| | AAAGCATAGAATCTTCTCTCTCATTGGTGGTAACGACTACAAAGACGATGACGACAAGTAAAGCGCT |
| | TCTAGAAGTTGTCTCCTCCTGCACTGACTGACTGATACAATCGATTTCTGGATCCGCAGGCCTAATC |
| | AACCTCTGGATTACAAAATTTGTGAAAGATTGACTGGTATTCTTAACTATGTTGCTCCTTTTACGCT |
| | ATGTGGATACGCTGCTTTAATGCCTTTGTATCATGCTATTGCTTCCCGTATGGCTTTCATTTTCTCC |
| | TCCTTGTATAAATCCTGGTTGCTGTCTCTTTATGAGGAGTTGTGGCCCGTTGTCAGGCAACGTGGCG |
| | TGGTGTGCACTGTGTTTGCTGACGCAACCCCCACTGGTTGGGGCATTGCCACCACCTGTCAGCTCCT |
| | TTCCGGGACTTTCGCTTTCCCCCTCCCTATTGCCACGGCGGAACTCATCGCCGCCTGCCTTGCCCGC |
| | TGCTGGACAGGGGCTCGGCTGTTGGGCACTGACAATTCCGTGGTGTTGTCGGGGAAGCTGACGTCCT |
| | TTCCATGGCTGCTCGCCTGTGTTGCCACCTGGATTCTGCGCGGGACGTCCTTCTGCTACGTCCCTTC |
| | GGCCCTCAATCCAGCGGACCTTCCTTCCCGCtgagagacacaaaaaattccaacacactattgcaat |
| | gaaaataaatttcctttattagccagaagtcagatgctcaaggggcttcatgatgtccccataattt |
| | ttggcagagggaaaaagatctcagtggtatttgtgagccagggcattggccttctgataggcagcct |
| | gcacctgaggagtgcggccgctttacttgtacagctcgtccatgccgagagtgatcccggcggcggt |
| | cacgaactccagcaggaccatgtgatcgcgcttctcgttggggtctttgctcagggcggactgggtg |
| | ctcaggtagtggttgtcgggcagcagcacggggccgtcgccgatgggggtgttctgctggtagtggt |
| | cggcgagctgcacgctgccgtcctcgatgttgtggcggatcttgaagttcaccttgatgccgttctt |
| | ctgcttgtcggccatgatatagacgttgtggctgttgtagttgtactccagcttgtgccccaggatg |
| | ttgccgtcctccttgaagtcgatgcccttcagctcgatgcggttcaccagggtgtcgccctcgaact |
| | tcacctcggcgcgggtcttgtagttgccgtcgtccttgaagaagatggtgcgctcctggacgtagcc |
| | ttcgggcatggcggacttgaagaagtcgtgctgcttcatgtggtcggggtagcggctgaagcactgc |
| | acgccgtaggtcagggtggtcacgagggtgggccagggcacgggcagcttgccggtggtgcagatga |
| | acttcagggtcagcttgccgtaggtggcatcgccctcgccctcgccggacacgctgaacttgtggcc |
| | gtttacgtcgccgtccagctcgaccaggatgggcaccaccccggtgaacagctcctcgcccttgctc |
| | accatggtggcgggatctgacggttcactaaaccagctctgcttatatagacctcccaccgtacacg |
| | cctaccgcccatttgcgtcaatggggcggagttgttacgacattttggaaagtcccgttgattttgg |
| | tgccaaaacaaactcccattgacgtcaatggggtggagacttggaaatccccgtgagtcaaaccgct |
| | atccacgcccattgatgtactgccaaaaccgcatcaccatggtaatagcgatgactaatacgtagat |
| | gtactgccaagtaggaaagtcccataaggtcatgtactgggcataatgccaggcgggccatttaccg |
| | tcattgacgtcaatagggggcgtacttggcatatgatacacttgatgtactgccaagtgggcagttt |
| | accgtaaatactccacccattgacgtcaatggaaagtccctattggcgttactatgggaacatacgt |
| | cattattgacgtcaatgggcgggggtcgttgggcggtcagccaggcgggccatttaccgtaagttat |
| | gtaacgGGCCTGCTGCCGGCTCTGCGGCCTCTTCCGCGTCTTCGCCTTCGCCCTCAGACGAGTCGGA |
| | TCTCCCTTTGGGCCGCCTCCCCGCCTGTCTAGCTTGACTGACTGAGATACAGCGTACCTTCAGCTCA |
| | CAGACATGATAAGATACATTGATGAGTTTGGACAAACCACAACTAGAATGCAGTGAAAAAAATGCTT |
| | TATTTGTGAAATTTGTGATGCTATTGCTTTATTTGTAACCATTATAAGCTGCAATAAACAAGTTAAC |
| | AACAACAATTGCATTCATTTTATGTTTCAGGTTCAGGGGGAGGTGTGGGAGGTTTTTTAAAGCAAGT |
| | AAAACCTCTACAAATGTGGTATTGGCCCATCTCTATCGGTATCGTAGCATAACCCCTTGGGGCCTCT |
| | AAACGGGTCTTGAGGGGTTTTTTGTGCCCCTCGGGCCGGATTGCTATCTACCGGCATTGGCGCAGAA |
| | AAAAATGCCTGATGCGACGCTGCGCGTCTTATACTCCCACATATGCCAGATTCAGCAACGGATACGG |
| | CTTCCCCAACTTGCCCACTTCCATACGTGTCCTCCTTACCAGAAATTTATCCTTAAGGTCGTCAGCT |
| | ATCCTGCAGGCGATCTCTCGATTTCGATCAAGACATTCCTTTAATGGTCTTTTCTGGACACCACTAG |
| | GGGTCAGAAGTAGTTCATCAAACTTTCTTCCCTCCCTAATCTCATTGGTTACCTTGGGCTATCGAAA |
| | CTTAATTAAGCGATCTGCATCTCAATTAGTCAGCAACCATAGTCCCGCCCCTAACTCCGCCCATCCC |
| | GCCCCTAACTCCGCCCAGTTCCGCCCATTCTCCGCCCCATCGCTGACTAATTTTTTTTATTTATGCA |
| | GAGGCCGAGGCCGCCTCGGCCTCTGAGCTATTCCAGAAGTAGTGAGGAGGCTTTTTTGGAGGCCTAG |
| | GCTTTTGCAAAGGAGGTAGCCAACATGATTGAACAAGATGGATTGCACGCAGGTTCTCCCGCCGCTT |
| | GGGTGGAGAGGCTATTCGGCTATGACTGGGCACAACAGACAATCGGCTGCTCTGATGCCGCCGTGTT |
| | CCGGCTGTCAGCGCAGGGGCGCCCGGTTCTTTTTGTCAAGACCGACCTGTCCGGTGCCCTGAATGAA |
| | CTCCAGGACGAGGCAGCGCGGCTATCGTGGCTGGCCACGACGGGCGTTCCTTGCGCAGCTGTGCTCG |
| | ACGTTGTCACTGAAGCGGGAAGGGACTGGCTGCTATTGGGCGAAGTGCCGGGGCAGGATCTCCTGTC |
| | ATCTCACCTTGCTCCTGCCGAGAAAGTATCCATCATGGCTGATGCAATGCGGCGGCTGCATACGCTT |
| | GATCCGGCTACCTGCCCATTCGACCACCAAGCGAAACATCGCATCGAGCGAGCACGTACTCGGATGG |
| | AAGCCGGTCTTGTCGATCAGGATGATCTGGACGAAGAGCATCAGGGGCTCGCGCCAGCCGAACTGTT |
| | CGCCAGGCTCAAGGCGCGGATGCCCGACGGCGAGGATCTCGTCGTGACCCACGGCGATGCCTGCTTG |
| | CCGAATATCATGGTGGAAAATGGCCGCTTTTCTGGATTCATCGACTGTGGCCGGCTGGGTGTGGCGG |
| | ACCGCTATCAGGACATAGCGTTGGCTACCCGTGATATTGCTGAAGAGCTTGGCGGCGAATGGGCTGA |
| | CCGCTTCCTCGTGCTTTACGGTATCGCCGCTCCCGATTCGCAGCGCATCGCCTTCTATCGCCTTCTT |
| | GACGAGTTCTTCTAGTATGTAAGCCCTGTGCCTTCTAGTTGCCAGCCATCTGTTGTTTGCCCCTCCC |
| | CCGTGCCTTCCTTGACCCTGGAAGGTGCCACTCCCACTGTCCTTTCCTAATAAAATGAGGAAATTGC |
| | ATCGCATTGTCTGAGTAGGTGTCATTCTATTCTGGGGGGTGGGGTGGGGCAGGACAGCAAGGGGGAG |
| | GATTGGGAAGACAATAGCAGGCATGCTGGGGATGCGGTGGGCTCTATGGTTAATTAACCAGTCAAGT |
| | CAGCTACTTGGCGAGATCGACTTGTCTGGGTTTCGACTACGCTCAGAATTGCGTCAGTCAAGTTCGA |
| | TCTGGTCCTTGCTATTGCACCCGTTCTCCGATTACGAGTTTCATTTAAATCATGTGAGCAAAAGGCC |
| | AGCAAAAGGCCAGGAACCGTAAAAAGGCCGCGTTGCTGGCGTTTTTCCATAGGCTCCGCCCCCCTGA |
| | CGAGCATCACAAAAATCGACGCTCAAGTCAGAGGTGGCGAAACCCGACAGGACTATAAAGATACCAG |
| | GCGTTTCCCCCTGGAAGCTCCCTCGTGCGCTCTCCTGTTCCGACCCTGCCGCTTACCGGATACCTGT |
| | CCGCCTTTCTCCCTTCGGGAAGCGTGGCGCTTTCTCATAGCTCACGCTGTAGGTATCTCAGTTCGGT |
| | GTAGGTCGTTCGCTCCAAGCTGGGCTGTGTGCACGAACCCCCCGTTCAGCCCGACCGCTGCGCCTTA |
| | TCCGGTAACTATCGTCTTGAGTCCAACCCGGTAAGACACGACTTATCGCCACTGGCAGCAGCCACTG |
| | GTAACAGGATTAGCAGAGCGAGGTATGTAGGCGGTGCTACAGAGTTCTTGAAGTGGTGGCCTAACTA |
| | CGGCTACACTAGAAGAACAGTATTTGGTATCTGCGCTCTGCTGAAGCCAGTTACCTTCGGAAAAAGA |
| | GTTGGTAGCTCTTGATCCGGCAAACAAACCACCGCTGGTAGCGGTGGTTTTTTTGTTTGCAAGCAGC |
| | AGATTACGCGCAGAAAAAAAGGATCTCAAGAAGATCCTTTGATCTTTTCTACGGGGTCTGACGCTCA |
| | GTGGAACGAAAACTCACGTTAAGGGATTTTGGTCATGAGATTATCAAAAAGGATCTTCACCTAGATC |
| | CTTTTAAATTAAAAATGAAGTTTTAAATCAATCTAAAGTATATATGAGTAAACTTGGTCTGACAGTT |
| | ACCAATGCTTAATCAGTGAGGCACCTATCTCAGCGATCTGTCTATTTCGTTCATCCATAGTTGCATT |
| | TAAATTTCCGAACTCTCCAAGGCCCTCGTCGGAAAATCTTCAAACCTTTCGTCCGATCCATCTTGCA |
| | GGCTACCTCTCGAACGAACTATCGCAAGTCTCTTGGCCGGCCTTGCGCCTTGGCTATTGCTTGGCAG |
| | CGCCTATCGCCAGGTATTACTCCAATCCCGAATATCCGAGATCGGGATCACCCGAGAGAAGTTCAAC |
| | CTACATCCTCAATCCCGATCTATCCGAGATCCGAGGAATA (SEQ ID NO: 94) |
| RET- | TATCGAAATCGGGGCGCGCCTGGTGTACCGAGAACGATCCTCTCAGTGCGAGTCTCGACGATCCATA |
| 005 | TCGTTGCTTGGCAGTCAGCCAGTCGGAATCCAGCTTGGGACCCAGGAAGTCCAATCGTCAGATATTG |
| LINE1- | TACTCAAGCCTGGTCACGGCAGCGTACCGATCTGTTTAAACCTAGATATTGATAGTCTGATCGGTCA |
| GFP | ACGTATAATCGAGTCCTAGCTTTTGCAAACATCTATCAAGAGACAGGATCAGCAGGAGGCTTTCGCA |
| ORF1-C | TGAGTATTCAACATTTCCGTGTCGCCCTTATTCCCTTTTTTGCGGCATTTTGCCTTCCTGTTTTTGC |
| Linker_ | TCACCCAGAAACGCTGGTGAAAGTAAAAGATGCTGAAGATCAGTTGGGTGCGCGAGTGGGTTACATC |
| Nucleo- | GAACTGGATCTCAACAGCGGTAAGATCCTTGAGAGTTTTCGCCCCGAAGAACGCTTTCCAATGATGA |
| plasmin- | GCACTTTTAAAGTTCTGCTATGTGGCGCGGTATTATCCCGTATTGACGCCGGGCAAGAGCAACTCGG |
| NLS | TCGCCGCATACACTATTCTCAGAATGACTTGGTTGAGTATTCACCAGTCACAGAAAAGCATCTTACG |
| | GATGGCATGACAGTAAGAGAATTATGCAGTGCTGCCATAACCATGAGTGATAACACTGCGGCCAACT |
| | TACTTCTGACAACGATTGGAGGACCGAAGGAGCTAACCGCTTTTTTGCACAACATGGGGGATCATGT |
| | AACTCGCCTTGATCGTTGGGAACCGGAGCTGAATGAAGCCATACCAAACGACGAGCGTGACACCACG |
| | ATGCCTGTAGCAATGGCAACAACCTTGCGTAAACTATTAACTGGCGAACTACTTACTCTAGCTTCCC |
| | GGCAACAGTTGATAGACTGGATGGAGGCGGATAAAGTTGCAGGACCACTTCTGCGCTCGGCCCTTCC |
| | GGCTGGCTGGTTTATTGCTGATAAATCTGGAGCCGGTGAGCGTGGGTCTCGCGGTATCATTGCAGCA |
| | CTGGGGCCAGATGGTAAGCCCTCCCGTATCGTAGTTATCTACACGACGGGGAGTCAGGCAACTATGG |
| | ATGAACGAAATAGACAGATCGCTGAGATAGGTGCCTCACTGATTAAGCATTGGTAACCGATTCTAGG |
| | TGCATTGGCGCAGAAAAAAATGCCTGATGCGACGCTGCGCGTCTTATACTCCCACATATGCCAGATT |
| | CAGCAACGGATACGGCTTCCCCAACTTGCCCACTTCCATACGTGTCCTCCTTACCAGAAATTTATCC |
| | TTAAGATCGTTTAAACTCGACTCTGGCTCTATCGAATCTCCGTCGTTTCGAGCTTACGCGAACAGCC |
| | GTGGCGCTCATTTGCTCGTCGGGCATCGAATCTCGTCAGCTATCGTCAGCTTACCTTTTTGGCAGCG |
| | ATCGCGGCTCCCGACATCTTGGACCATTAGCTCCACAGGTATCTTCTTCCCTCTAGTGGTCATAACA |
| | GCAGCTTCAGCTACCTCTCAATTCAAAAAACCCCTCAAGACCCGTTTAGAGGCCCCAAGGGGTTATG |
| | CTATCAATCGTTGCGTTACACACACAAAAAACCAACACACATCCATCTTCGATGGATAGCGATTTTA |
| | TTATCTAACTGCTGATCGAGTGTAGCCAGATCTAGTAATCAATTACGGGGTCATTAGTTCATAGCCC |
| | ATATATGGAGTTCCGCGTTACATAACTTACGGTAAATGGCCCGCCTGGCTGACCGCCCAACGACCCC |
| | CGCCCATTGACGTCAATAATGACGTATGTTCCCATAGTAACGCCAATAGGGACTTTCCATTGACGTC |
| | AATGGGTGGAGTATTTACGGTAAACTGCCCACTTGGCAGTACATCAAGTGTATCATATGCCAAGTAC |
| | GCCCCCTATTGACGTCAATGACGGTAAATGGCCCGCCTGGCATTATGCCCAGTACATGACCTTATGG |
| | GACTTTCCTACTTGGCAGTACATCTACGTATTAGTCATCGCTATTACCATGCTGATGCGGTTTTGGC |
| | AGTACATCAATGGGCGTGGATAGCGGTTTGACTCACGGGGATTTCCAAGTCTCCACCCCATTGACGT |
| | CAATGGGAGTTTGTTTTGGCACCAAAATCAACGGGACTTTCCAAAATGTCGTAACAACTCCGCCCCA |
| | TTGACGCAAATGGGCGGTAGGCGTGTACGGTGGGAGGTCTATATAAGCAGAGCTGGTTTAGTGAACC |
| | GTCAGATCAGATCTTTGTCGATCCTACCATCCACTCGACACACCCGCCAGCGGCCGCTAATACGACT |
| | CACTATAGGGAGAAGTACTGCCACCATGGGCAAGAAGCAAAATCGCAAGACGGGGAATTCCAAGACA |
| | CAATCCGCTAGCCCACCACCTAAAGAGCGTTCTAGCTCCCCTGCTACTGAGCAGTCCTGGATGGAAA |
| | ACGACTTCGATGAACTCCGGGAAGAGGGATTTAGGCGATCCAACTATTCAGAACTCCGCGAAGATAT |
| | CCAGACAAAGGGGAAGGAAGTCGAGAATTTCGAGAAGAACCTCGAGGAGTGCATCACCCGTATCACA |
| | AACACTGAGAAATGTCTCAAAGAACTCATGGAACTTAAGACAAAAGCCAGGGAGCTTCGAGAGGAGT |
| | GTCGGAGTCTGAGATCCAGGTGTGACCAGCTCGAGGAGCGCGTGAGCGCGATGGAAGACGAGATGAA |
| | CGAGATGAAAAGAGAGGGCAAATTCAGGGAGAAGCGCATTAAGAGGAACGAACAGAGTCTGCAGGAG |
| | ATTTGGGATTACGTCAAGAGGCCTAACCTGCGGTTGATCGGCGTCCCCGAGAGCGACGTAGAAAACG |
| | GGACTAAACTGGAGAATACACTTCAAGACATCATTCAAGAAAATTTTCCAAACCTGGCTCGGCAAGC |
| | TAATGTGCAAATCCAAGAGATCCAACGCACACCCCAGCGGTATAGCTCTCGGCGTGCCACCCCTAGG |
| | CATATTATCGTGCGCTTTACTAAGGTGGAGATGAAAGAGAAGATGCTGCGAGCCGCTCGGGAAAAGG |
| | GAAGGGTGACTTTGAAGGGCAAACCTATTCGGCTGACGGTTGACCTTAGCGCCGAGACACTCCAGGC |
| | ACGCCGGGAATGGGGCCCCATCTTTAATATCCTGAAGGAGAAGAACTTCCAGCCACGAATCTCTTAC |
| | CCTGCAAAGTTGAGTTTTATCTCCGAGGGTGAGATTAAGTATTTCATCGATAAACAGATGCTGCGAG |
| | ACTTCGTGACAACTCGCCCAGCTCTCAAGGAACTGCTCAAAGAGGCTCTTAATATGGAGCGCAATAA |
| | TAGATATCAACCCTTGCAGAACCACGCAAAGATGggcggcggcagcaaaaggccggcggccacgaaa |
| | aaggccggccaggcaaaaaagaaaaagTGAGACAGCCGTCAGACCATCAAGACTAGGAAGAAACTGC |
| | ATCAACTAATGAGCAAAATCACCAGCTAACATCATAGTATACATGACCGGCTCTAACTCACATATCA |
| | CCATCCTTACACTTAACATTAACGGCCTCAACTCAGCTATCAAGCGCCATCGGCTGGCCAGCTGGAT |
| | CAAATCACAGGATCCAAGCGTTTGTTGCATCCAAGAGACCCACCTGACCTGTAGAGATACTCACCGC |
| | CTCAAGATCAAGGGATGGCGAAAGATTTATCAGGCGAACGGTAAGCAGAAGAAAGCCGGAGTCGCAA |
| | TTCTGGTCTCAGACAAGACGGATTTCAAGCCCACCAAAATTAAGCGTGATAAGGAAGGTCACTATAT |
| | TATGGTGAAAGGCAGCATACAGCAGGAAGAACTTACCATATTGAACATCTACGCGCCAAACACCGGC |
| | GCACCTCGCTTTATCAAACAGGTCCTGTCCGATCTGCAGCGAGATCTGGATTCTCATACGTTGATTA |
| | TGGGTGATTTCAATACACCATTGAGCACCCTGGATCGCAGCACCAGGCAAAAGGTAAATAAAGACAC |
| | GCAAGAGCTCAATAGCGCACTGCATCAGGCAGATCTCATTGATATTTATCGCACTCTTCATCCTAAG |
| | AGTACCGAGTACACATTCTTCAGCGCCCCACATCATACATACTCAAAGATCGATCATATCGTCGGCT |
| | CAAAGGCTCTGCTGTCAAAGTGCAAGCGCACAGAGATAATTACAAATTACCTGTCAGATCATAGCGC |
| | GATCAAGCTCGAGCTGAGAATCAAGAACCTGACCCAGAGCCGGAGTACCACTTGGAAGCTTAATAAC |
| | CTGCTGCTCAACGATTATTGGGTCCACAATGAGATGAAGGCAGAGATTAAAATGTTCTTCGAAACAA |
| | ATGAGAATAAGGATACTACCTATCAAAACCTTTGGGATGCCTTTAAGGCCGTCTGCAGAGGCAAGTT |
| | CATCGCCCTCAACGCCTATAAAAGAAAACAAGAGAGATCTAAGATCGATACTCTCACCTCTCAGCTG |
| | AAGGAGTTGGAGAAACAGGAACAGACCCACTCCAAGGCGTCAAGACGGCAGGAGATCACAAAGATTC |
| | GCGCCGAGTTGAAAGAGATCGAAACCCAAAAGACTCTTCAGAAAATTAACGAGTCTCGTAGTTGGTT |
| | CTTCGAGCGGATTAATAAGATAGACAGACCTCTGGCACGACTGATTAAGAAGAAGCGCGAAAAGAAC |
| | CAGATTGATACCATCAAGAACGACAAGGGCGACATCACTACTGACCCGACCGAGATCCAGACCACTA |
| | TTCGGGAGTATTATAAGCATTTGTATGCTAACAAGCTTGAGAACCTGGAAGAGATGGACACTTTTCT |
| | GGATACCTATACTCTGCCACGGCTTAATCAAGAGGAAGTCGAGTCCCTCAACCGCCCAATTACAGGA |
| | AGCGAGATTGTGGCCATAATTAACTCCCTGCCGACAAAGAAATCTCCTGGTCCGGACGGGTTTACAG |
| | CTGAGTTTTATCAACGGTATATGGAAGAGCTTGTACCGTTTCTGCTCAAGCTCTTTCAGTCTATAGA |
| | AAAGGAAGGCATCTTGCCCAATTCCTTCTACGAAGCTTCTATAATACTTATTCCCAAACCAGGACGC |
| | GATACCACAAAGAAGGAAAACTTCCGGCCCATTAGTCTCATGAATATCGACGCTAAAATATTGAACA |
| | AGATTCTCGCCAACAGAATCCAACAACATATTAAGAAATTGATACATCACGACCAGGTGGGGTTTAT |
| | ACCTGGCATGCAGGGCTGGTTTAACATCCGGAAGAGTATTAACGTCATTCAACACATTAATAGAGCT |
| | AAGGATAAGAATCATATGATCATCTCTATAGACGCGGAAAAGGCATTCGATAAGATTCAGCAGCCAT |
| | TTATGCTCAAGACTCTGAACAAACTCGGCATCGACGGAACATATTTTAAGATTATTCGCGCAATTTA |
| | CGATAAGCCGACTGCTAACATTATCCTTAACGGCCAAAAGCTCGAGGCCTTTCCGCTCAAGACTGGA |
| | ACCCGCCAAGGCTGTCCCCTCTCCCCGCTTTTGTTTAATATTGTACTCGAGGTGCTGGCTAGGGCTA |
| | TTCGTCAAGAGAAAGAGATTAAAGGGATACAGCTCGGGAAGGAAGAGGTCAAGCTTTCCTTGTTCGC |
| | CGATGATATGATTGTGTACCTGGAGAATCCTATTGTGTCTGCTCAGAACCTTCTTAAACTTATTTCT |
| | AACTTTAGCAAGGTCAGCGGCTATAAGATTAACGTCCAGAAATCTCAGGCCTTTCTGTACACAAATA |
| | ATCGACAGACCGAATCCCAGATAATGGGTGAGCTTCCGTTTGTCATAGCCAGCAAAAGGATAAAGTA |
| | TCTCGGAATCCAGCTGACACGAGACGTTAAAGATTTGTTTAAGGAAAATTACAAGCCTCTCCTGAAA |
| | GAGATTAAGGAAGATACTAATAAGTGGAAGAATATCCCCTGTTCATGGGTTGGCAGAATCAACATAG |
| | TGAAGATGGCAATACTTCCTAAAGTGATATATCGCTTTAACGCCATCCCAATTAAACTGCCTATGAC |
| | CTTCTTTACGGAGCTCGAGAAAACAACCCTTAAATTTATATGGAATCAAAAGAGAGCAAGAATAGCG |
| | AAGTCCATCTTGAGCCAGAAGAATAAGGCCGGTGGGATTACTTTGCCTGATTTTAAGTTGTATTATA |
| | AAGCCACAGTAACTAAGACAGCCTGGTATTGGTATCAGAATAGAGACATCGACCAGTGGAATCGGAC |
| | CGAACCATCAGAGATAATGCCCCACATCTATAATTACCTTATATTCGATAAGCCAGAAAAGAATAAA |
| | CAGTGGGGCAAAGACAGCCTCTTCAACAAGTGGTGTTGGGAGAATTGGCTGGCCATATGCCGGAAAC |
| | TCAAGCTCGACCCCTTTCTTACACCCTACACTAAAATCAACAGTAGGTGGATCAAGGACTTGAATGT |
| | CAAGCCAAAGACTATAAAGACACTGGAAGAGAATCTTGGGATCACAATACAAGATATAGGCGTCGGC |
| | AAAGATTTTATGTCAAAGACGCCCAAGGCCATGGCCACTAAGGATAAGATTGATAAGTGGGACCTTA |
| | TTAAGCTCAAAAGCTTCTGTACTGCCAAGGAGACCACGATCAGAGTTAATAGGCAGCCCACTACATG |
| | GGAAAAGATTTTCGCCACTTATTCATCAGATAAGGGGTTGATAAGCAGAATATATAACGAGCTGAAG |
| | CAGATCTACAAGAAGAAAACGAATAATCCCATCAAGAAGTGGGCAAAAGATATGAACAGGCATTTTA |
| | GCAAAGAGGATATCTACGCCGCGAAGAAGCATATGAAGAAGTGTAGTTCAAGCTTGGCCATTCGTGA |
| | GATGCAGATTAAGACGACCATGCGATACCACCTTACCCCAGTGAGGATGGCAATTATCAAGAAATCT |
| | GGCAATAATAGATGTTGGCGGGGCTGTGGCGAGATTGGCACCCTGCTCCATTGCTGGTGGGATTGCA |
| | AGCTGGTGCAGCCGCTTTGGAAATCAGTCTGGCGCTTTCTGAGGGACCTCGAGCTTGAGATTCCCTT |
| | CGATCCCGCAATTCCCTTGCTCGGAATCTATCCTAACGAATACAAGAGCTGTTGTTACAAGGATACG |
| | TGTACCCGGATGTTCATCGCGGCCTTGTTTACGATAGCTAAGACGTGGAATCAGCCTAAGTGCCCCA |
| | CAATGATCGATTGGATCAAGAAAATGTGGCATATTTATACCATGGAGTATTACGCAGCAATTAAGAA |
| | TGACGAATTTATTTCCTTCGTTGGGACCTGGATGAAGCTGGAGACTATTATTCTGAGCAAGCTGTCT |
| | CAGGAGCAAAAGACAAAGCATAGAATCTTCTCTCTCATTGGTGGTAACGACTACAAAGACGATGACG |
| | ACAAGTAAAGCGCTTCTAGAAGTTGTCTCCTCCTGCACTGACTGACTGATACAATCGATTTCTGGAT |
| | CCGCAGGCCTAATCAACCTCTGGATTACAAAATTTGTGAAAGATTGACTGGTATTCTTAACTATGTT |
| | GCTCCTTTTACGCTATGTGGATACGCTGCTTTAATGCCTTTGTATCATGCTATTGCTTCCCGTATGG |
| | CTTTCATTTTCTCCTCCTTGTATAAATCCTGGTTGCTGTCTCTTTATGAGGAGTTGTGGCCCGTTGT |
| | CAGGCAACGTGGCGTGGTGTGCACTGTGTTTGCTGACGCAACCCCCACTGGTTGGGGCATTGCCACC |
| | ACCTGTCAGCTCCTTTCCGGGACTTTCGCTTTCCCCCTCCCTATTGCCACGGCGGAACTCATCGCCG |
| | CCTGCCTTGCCCGCTGCTGGACAGGGGCTCGGCTGTTGGGCACTGACAATTCCGTGGTGTTGTCGGG |
| | GAAGCTGACGTCCTTTCCATGGCTGCTCGCCTGTGTTGCCACCTGGATTCTGCGCGGGACGTCCTTC |
| | TGCTACGTCCCTTCGGCCCTCAATCCAGCGGACCTTCCTTCCCGCtgagagacacaaaaaattccaa |
| | cacactattgcaatgaaaataaatttcctttattagccagaagtcagatgctcaaggggcttcatga |
| | tgtccccataatttttggcagagggaaaaagatctcagtggtatttgtgagccagggcattggcctt |
| | ctgataggcagcctgcacctgaggagtgcggccgctttacttgtacagctcgtccatgccgagagtg |
| | atcccggcggcggtcacgaactccagcaggaccatgtgatcgcgcttctcgttggggtctttgctca |
| | gggcggactgggtgctcaggtagtggttgtcgggcagcagcacggggccgtcgccgatgggggtgtt |
| | ctgctggtagtggtcggcgagctgcacgctgccgtcctcgatgttgtggcggatcttgaagttcacc |
| | ttgatgccgttcttctgcttgtcggccatgatatagacgttgtggctgttgtagttgtactccagct |
| | tgtgccccaggatgttgccgtcctccttgaagtcgatgcccttcagctcgatgcggttcaccagggt |
| | gtcgccctcgaacttcacctcggcgcgggtcttgtagttgccgtcgtccttgaagaagatggtgcgc |
| | tcctggacgtagccttcgggcatggcggacttgaagaagtcgtgctgcttcatgtggtcggggtagc |
| | ggctgaagcactgcacgccgtaggtcagggtggtcacgagggtgggccagggcacgggcagcttgcc |
| | ggtggtgcagatgaacttcagggtcagcttgccgtaggtggcatcgccctcgccctcgccggacacg |
| | ctgaacttgtggccgtttacgtcgccgtccagctcgaccaggatgggcaccaccccggtgaacagct |
| | cctcgcccttgctcaccatggtggcgggatctgacggttcactaaaccagctctgcttatatagacc |
| | tcccaccgtacacgcctaccgcccatttgcgtcaatggggcggagttgttacgacattttggaaagt |
| | cccgttgattttggtgccaaaacaaactcccattgacgtcaatggggtggagacttggaaatccccg |
| | tgagtcaaaccgctatccacgcccattgatgtactgccaaaaccgcatcaccatggtaatagcgatg |
| | actaatacgtagatgtactgccaagtaggaaagtcccataaggtcatgtactgggcataatgccagg |
| | cgggccatttaccgtcattgacgtcaatagggggcgtacttggcatatgatacacttgatgtactgc |
| | caagtgggcagtttaccgtaaatactccacccattgacgtcaatggaaagtccctattggcgttact |
| | atgggaacatacgtcattattgacgtcaatgggcgggggtcgttgggcggtcagccaggcgggccat |
| | ttaccgtaagttatgtaacgGGCCTGCTGCCGGCTCTGCGGCCTCTTCCGCGTCTTCGCCTTCGCCC |
| | TCAGACGAGTCGGATCTCCCTTTGGGCCGCCTCCCCGCCTGTCTAGCTTGACTGACTGAGATACAGC |
| | GTACCTTCAGCTCACAGACATGATAAGATACATTGATGAGTTTGGACAAACCACAACTAGAATGCAG |
| | TGAAAAAAATGCTTTATTTGTGAAATTTGTGATGCTATTGCTTTATTTGTAACCATTATAAGCTGCA |
| | ATAAACAAGTTAACAACAACAATTGCATTCATTTTATGTTTCAGGTTCAGGGGGAGGTGTGGGAGGT |
| | TTTTTAAAGCAAGTAAAACCTCTACAAATGTGGTATTGGCCCATCTCTATCGGTATCGTAGCATAAC |
| | CCCTTGGGGCCTCTAAACGGGTCTTGAGGGGTTTTTTGTGCCCCTCGGGCCGGATTGCTATCTACCG |
| | GCATTGGCGCAGAAAAAAATGCCTGATGCGACGCTGCGCGTCTTATACTCCCACATATGCCAGATTC |
| | AGCAACGGATACGGCTTCCCCAACTTGCCCACTTCCATACGTGTCCTCCTTACCAGAAATTTATCCT |
| | TAAGGTCGTCAGCTATCCTGCAGGCGATCTCTCGATTTCGATCAAGACATTCCTTTAATGGTCTTTT |
| | CTGGACACCACTAGGGGTCAGAAGTAGTTCATCAAACTTTCTTCCCTCCCTAATCTCATTGGTTACC |
| | TTGGGCTATCGAAACTTAATTAAGCGATCTGCATCTCAATTAGTCAGCAACCATAGTCCCGCCCCTA |
| | ACTCCGCCCATCCCGCCCCTAACTCCGCCCAGTTCCGCCCATTCTCCGCCCCATCGCTGACTAATTT |
| | TTTTTATTTATGCAGAGGCCGAGGCCGCCTCGGCCTCTGAGCTATTCCAGAAGTAGTGAGGAGGCTT |
| | TTTTGGAGGCCTAGGCTTTTGCAAAGGAGGTAGCCAACATGATTGAACAAGATGGATTGCACGCAGG |
| | TTCTCCCGCCGCTTGGGTGGAGAGGCTATTCGGCTATGACTGGGCACAACAGACAATCGGCTGCTCT |
| | GATGCCGCCGTGTTCCGGCTGTCAGCGCAGGGGCGCCCGGTTCTTTTTGTCAAGACCGACCTGTCCG |
| | GTGCCCTGAATGAACTCCAGGACGAGGCAGCGCGGCTATCGTGGCTGGCCACGACGGGCGTTCCTTG |
| | CGCAGCTGTGCTCGACGTTGTCACTGAAGCGGGAAGGGACTGGCTGCTATTGGGCGAAGTGCCGGGG |
| | CAGGATCTCCTGTCATCTCACCTTGCTCCTGCCGAGAAAGTATCCATCATGGCTGATGCAATGCGGC |
| | GGCTGCATACGCTTGATCCGGCTACCTGCCCATTCGACCACCAAGCGAAACATCGCATCGAGCGAGC |
| | ACGTACTCGGATGGAAGCCGGTCTTGTCGATCAGGATGATCTGGACGAAGAGCATCAGGGGCTCGCG |
| | CCAGCCGAACTGTTCGCCAGGCTCAAGGCGCGGATGCCCGACGGCGAGGATCTCGTCGTGACCCACG |
| | GCGATGCCTGCTTGCCGAATATCATGGTGGAAAATGGCCGCTTTTCTGGATTCATCGACTGTGGCCG |
| | GCTGGGTGTGGCGGACCGCTATCAGGACATAGCGTTGGCTACCCGTGATATTGCTGAAGAGCTTGGC |
| | GGCGAATGGGCTGACCGCTTCCTCGTGCTTTACGGTATCGCCGCTCCCGATTCGCAGCGCATCGCCT |
| | TCTATCGCCTTCTTGACGAGTTCTTCTAGTATGTAAGCCCTGTGCCTTCTAGTTGCCAGCCATCTGT |
| | TGTTTGCCCCTCCCCCGTGCCTTCCTTGACCCTGGAAGGTGCCACTCCCACTGTCCTTTCCTAATAA |
| | AATGAGGAAATTGCATCGCATTGTCTGAGTAGGTGTCATTCTATTCTGGGGGGTGGGGTGGGGCAGG |
| | ACAGCAAGGGGGAGGATTGGGAAGACAATAGCAGGCATGCTGGGGATGCGGTGGGCTCTATGGTTAA |
| | TTAACCAGTCAAGTCAGCTACTTGGCGAGATCGACTTGTCTGGGTTTCGACTACGCTCAGAATTGCG |
| | TCAGTCAAGTTCGATCTGGTCCTTGCTATTGCACCCGTTCTCCGATTACGAGTTTCATTTAAATCAT |
| | GTGAGCAAAAGGCCAGCAAAAGGCCAGGAACCGTAAAAAGGCCGCGTTGCTGGCGTTTTTCCATAGG |
| | CTCCGCCCCCCTGACGAGCATCACAAAAATCGACGCTCAAGTCAGAGGTGGCGAAACCCGACAGGAC |
| | TATAAAGATACCAGGCGTTTCCCCCTGGAAGCTCCCTCGTGCGCTCTCCTGTTCCGACCCTGCCGCT |
| | TACCGGATACCTGTCCGCCTTTCTCCCTTCGGGAAGCGTGGCGCTTTCTCATAGCTCACGCTGTAGG |
| | TATCTCAGTTCGGTGTAGGTCGTTCGCTCCAAGCTGGGCTGTGTGCACGAACCCCCCGTTCAGCCCG |
| | ACCGCTGCGCCTTATCCGGTAACTATCGTCTTGAGTCCAACCCGGTAAGACACGACTTATCGCCACT |
| | GGCAGCAGCCACTGGTAACAGGATTAGCAGAGCGAGGTATGTAGGCGGTGCTACAGAGTTCTTGAAG |
| | TGGTGGCCTAACTACGGCTACACTAGAAGAACAGTATTTGGTATCTGCGCTCTGCTGAAGCCAGTTA |
| | CCTTCGGAAAAAGAGTTGGTAGCTCTTGATCCGGCAAACAAACCACCGCTGGTAGCGGTGGTTTTTT |
| | TGTTTGCAAGCAGCAGATTACGCGCAGAAAAAAAGGATCTCAAGAAGATCCTTTGATCTTTTCTACG |
| | GGGTCTGACGCTCAGTGGAACGAAAACTCACGTTAAGGGATTTTGGTCATGAGATTATCAAAAAGGA |
| | TCTTCACCTAGATCCTTTTAAATTAAAAATGAAGTTTTAAATCAATCTAAAGTATATATGAGTAAAC |
| | TTGGTCTGACAGTTACCAATGCTTAATCAGTGAGGCACCTATCTCAGCGATCTGTCTATTTCGTTCA |
| | TCCATAGTTGCATTTAAATTTCCGAACTCTCCAAGGCCCTCGTCGGAAAATCTTCAAACCTTTCGTC |
| | CGATCCATCTTGCAGGCTACCTCTCGAACGAACTATCGCAAGTCTCTTGGCCGGCCTTGCGCCTTGG |
| | CTATTGCTTGGCAGCGCCTATCGCCAGGTATTACTCCAATCCCGAATATCCGAGATCGGGATCACCC |
| | GAGAGAAGTTCAACCTACATCCTCAATCCCGATCTATCCGAGATCCGAGGAA (SEQ ID NO: |
| | 95) |
| RET- | GTCGGAAAATCTTCAAACCTTTCGTCCGATCCATCTTGCAGGCTACCTCTCGAACGAACTATCGCAA |
| 006 | GTCTCTTGGCCGGCCTTGCGCCTTGGCTATTGCTTGGCAGCGCCTATCGCCAGGTATTACTCCAATC |
| LINE1- | CCGAATATCCGAGATCGGGATCACCCGAGAGAAGTTCAACCTACATCCTCAATCCCGATCTATCCGA |
| GFP | GATCCGAGGAATATCGAAATCGGGGCGCGCCTGGTGTACCGAGAACGATCCTCTCAGTGCGAGTCTC |
| ORF1- | GACGATCCATATCGTTGCTTGGCAGTCAGCCAGTCGGAATCCAGCTTGGGACCCAGGAAGTCCAATC |
| NSV40 | GTCAGATATTGTACTCAAGCCTGGTCACGGCAGCGTACCGATCTGTTTAAACCTAGATATTGATAGT |
| NLS | CTGATCGGTCAACGTATAATCGAGTCCTAGCTTTTGCAAACATCTATCAAGAGACAGGATCAGCAGG |
| | AGGCTTTCGCATGAGTATTCAACATTTCCGTGTCGCCCTTATTCCCTTTTTTGCGGCATTTTGCCTT |
| | CCTGTTTTTGCTCACCCAGAAACGCTGGTGAAAGTAAAAGATGCTGAAGATCAGTTGGGTGCGCGAG |
| | TGGGTTACATCGAACTGGATCTCAACAGCGGTAAGATCCTTGAGAGTTTTCGCCCCGAAGAACGCTT |
| | TCCAATGATGAGCACTTTTAAAGTTCTGCTATGTGGCGCGGTATTATCCCGTATTGACGCCGGGCAA |
| | GAGCAACTCGGTCGCCGCATACACTATTCTCAGAATGACTTGGTTGAGTATTCACCAGTCACAGAAA |
| | AGCATCTTACGGATGGCATGACAGTAAGAGAATTATGCAGTGCTGCCATAACCATGAGTGATAACAC |
| | TGCGGCCAACTTACTTCTGACAACGATTGGAGGACCGAAGGAGCTAACCGCTTTTTTGCACAACATG |
| | GGGGATCATGTAACTCGCCTTGATCGTTGGGAACCGGAGCTGAATGAAGCCATACCAAACGACGAGC |
| | GTGACACCACGATGCCTGTAGCAATGGCAACAACCTTGCGTAAACTATTAACTGGCGAACTACTTAC |
| | TCTAGCTTCCCGGCAACAGTTGATAGACTGGATGGAGGCGGATAAAGTTGCAGGACCACTTCTGCGC |
| | TCGGCCCTTCCGGCTGGCTGGTTTATTGCTGATAAATCTGGAGCCGGTGAGCGTGGGTCTCGCGGTA |
| | TCATTGCAGCACTGGGGCCAGATGGTAAGCCCTCCCGTATCGTAGTTATCTACACGACGGGGAGTCA |
| | GGCAACTATGGATGAACGAAATAGACAGATCGCTGAGATAGGTGCCTCACTGATTAAGCATTGGTAA |
| | CCGATTCTAGGTGCATTGGCGCAGAAAAAAATGCCTGATGCGACGCTGCGCGTCTTATACTCCCACA |
| | TATGCCAGATTCAGCAACGGATACGGCTTCCCCAACTTGCCCACTTCCATACGTGTCCTCCTTACCA |
| | GAAATTTATCCTTAAGATCGTTTAAACTCGACTCTGGCTCTATCGAATCTCCGTCGTTTCGAGCTTA |
| | CGCGAACAGCCGTGGCGCTCATTTGCTCGTCGGGCATCGAATCTCGTCAGCTATCGTCAGCTTACCT |
| | TTTTGGCAGCGATCGCGGCTCCCGACATCTTGGACCATTAGCTCCACAGGTATCTTCTTCCCTCTAG |
| | TGGTCATAACAGCAGCTTCAGCTACCTCTCAATTCAAAAAACCCCTCAAGACCCGTTTAGAGGCCCC |
| | AAGGGGTTATGCTATCAATCGTTGCGTTACACACACAAAAAACCAACACACATCCATCTTCGATGGA |
| | TAGCGATTTTATTATCTAACTGCTGATCGAGTGTAGCCAGATCTAGTAATCAATTACGGGGTCATTA |
| | GTTCATAGCCCATATATGGAGTTCCGCGTTACATAACTTACGGTAAATGGCCCGCCTGGCTGACCGC |
| | CCAACGACCCCCGCCCATTGACGTCAATAATGACGTATGTTCCCATAGTAACGCCAATAGGGACTTT |
| | CCATTGACGTCAATGGGTGGAGTATTTACGGTAAACTGCCCACTTGGCAGTACATCAAGTGTATCAT |
| | ATGCCAAGTACGCCCCCTATTGACGTCAATGACGGTAAATGGCCCGCCTGGCATTATGCCCAGTACA |
| | TGACCTTATGGGACTTTCCTACTTGGCAGTACATCTACGTATTAGTCATCGCTATTACCATGCTGAT |
| | GCGGTTTTGGCAGTACATCAATGGGCGTGGATAGCGGTTTGACTCACGGGGATTTCCAAGTCTCCAC |
| | CCCATTGACGTCAATGGGAGTTTGTTTTGGCACCAAAATCAACGGGACTTTCCAAAATGTCGTAACA |
| | ACTCCGCCCCATTGACGCAAATGGGCGGTAGGCGTGTACGGTGGGAGGTCTATATAAGCAGAGCTGG |
| | TTTAGTGAACCGTCAGATCAGATCTTTGTCGATCCTACCATCCACTCGACACACCCGCCAGCGGCCG |
| | CTAATACGACTCACTATAGGGAGAAGTACTGCCACCATGccaaagaagaagcggaaggtcGGCAAGA |
| | AGCAAAATCGCAAGACGGGGAATTCCAAGACACAATCCGCTAGCCCACCACCTAAAGAGCGTTCTAG |
| | CTCCCCTGCTACTGAGCAGTCCTGGATGGAAAACGACTTCGATGAACTCCGGGAAGAGGGATTTAGG |
| | CGATCCAACTATTCAGAACTCCGCGAAGATATCCAGACAAAGGGGAAGGAAGTCGAGAATTTCGAGA |
| | AGAACCTCGAGGAGTGCATCACCCGTATCACAAACACTGAGAAATGTCTCAAAGAACTCATGGAACT |
| | TAAGACAAAAGCCAGGGAGCTTCGAGAGGAGTGTCGGAGTCTGAGATCCAGGTGTGACCAGCTCGAG |
| | GAGCGCGTGAGCGCGATGGAAGACGAGATGAACGAGATGAAAAGAGAGGGCAAATTCAGGGAGAAGC |
| | GCATTAAGAGGAACGAACAGAGTCTGCAGGAGATTTGGGATTACGTCAAGAGGCCTAACCTGCGGTT |
| | GATCGGCGTCCCCGAGAGCGACGTAGAAAACGGGACTAAACTGGAGAATACACTTCAAGACATCATT |
| | CAAGAAAATTTTCCAAACCTGGCTCGGCAAGCTAATGTGCAAATCCAAGAGATCCAACGCACACCCC |
| | AGCGGTATAGCTCTCGGCGTGCCACCCCTAGGCATATTATCGTGCGCTTTACTAAGGTGGAGATGAA |
| | AGAGAAGATGCTGCGAGCCGCTCGGGAAAAGGGAAGGGTGACTTTGAAGGGCAAACCTATTCGGCTG |
| | ACGGTTGACCTTAGCGCCGAGACACTCCAGGCACGCCGGGAATGGGGCCCCATCTTTAATATCCTGA |
| | AGGAGAAGAACTTCCAGCCACGAATCTCTTACCCTGCAAAGTTGAGTTTTATCTCCGAGGGTGAGAT |
| | TAAGTATTTCATCGATAAACAGATGCTGCGAGACTTCGTGACAACTCGCCCAGCTCTCAAGGAACTG |
| | CTCAAAGAGGCTCTTAATATGGAGCGCAATAATAGATATCAACCCTTGCAGAACCACGCAAAGATGT |
| | GAGACAGCCGTCAGACCATCAAGACTAGGAAGAAACTGCATCAACTAATGAGCAAAATCACCAGCTA |
| | ACATCATAGTATACATGACCGGCTCTAACTCACATATCACCATCCTTACACTTAACATTAACGGCCT |
| | CAACTCAGCTATCAAGCGCCATCGGCTGGCCAGCTGGATCAAATCACAGGATCCAAGCGTTTGTTGC |
| | ATCCAAGAGACCCACCTGACCTGTAGAGATACTCACCGCCTCAAGATCAAGGGATGGCGAAAGATTT |
| | ATCAGGCGAACGGTAAGCAGAAGAAAGCCGGAGTCGCAATTCTGGTCTCAGACAAGACGGATTTCAA |
| | GCCCACCAAAATTAAGCGTGATAAGGAAGGTCACTATATTATGGTGAAAGGCAGCATACAGCAGGAA |
| | GAACTTACCATATTGAACATCTACGCGCCAAACACCGGCGCACCTCGCTTTATCAAACAGGTCCTGT |
| | CCGATCTGCAGCGAGATCTGGATTCTCATACGTTGATTATGGGTGATTTCAATACACCATTGAGCAC |
| | CCTGGATCGCAGCACCAGGCAAAAGGTAAATAAAGACACGCAAGAGCTCAATAGCGCACTGCATCAG |
| | GCAGATCTCATTGATATTTATCGCACTCTTCATCCTAAGAGTACCGAGTACACATTCTTCAGCGCCC |
| | CACATCATACATACTCAAAGATCGATCATATCGTCGGCTCAAAGGCTCTGCTGTCAAAGTGCAAGCG |
| | CACAGAGATAATTACAAATTACCTGTCAGATCATAGCGCGATCAAGCTCGAGCTGAGAATCAAGAAC |
| | CTGACCCAGAGCCGGAGTACCACTTGGAAGCTTAATAACCTGCTGCTCAACGATTATTGGGTCCACA |
| | ATGAGATGAAGGCAGAGATTAAAATGTTCTTCGAAACAAATGAGAATAAGGATACTACCTATCAAAA |
| | CCTTTGGGATGCCTTTAAGGCCGTCTGCAGAGGCAAGTTCATCGCCCTCAACGCCTATAAAAGAAAA |
| | CAAGAGAGATCTAAGATCGATACTCTCACCTCTCAGCTGAAGGAGTTGGAGAAACAGGAACAGACCC |
| | ACTCCAAGGCGTCAAGACGGCAGGAGATCACAAAGATTCGCGCCGAGTTGAAAGAGATCGAAACCCA |
| | AAAGACTCTTCAGAAAATTAACGAGTCTCGTAGTTGGTTCTTCGAGCGGATTAATAAGATAGACAGA |
| | CCTCTGGCACGACTGATTAAGAAGAAGCGCGAAAAGAACCAGATTGATACCATCAAGAACGACAAGG |
| | GCGACATCACTACTGACCCGACCGAGATCCAGACCACTATTCGGGAGTATTATAAGCATTTGTATGC |
| | TAACAAGCTTGAGAACCTGGAAGAGATGGACACTTTTCTGGATACCTATACTCTGCCACGGCTTAAT |
| | CAAGAGGAAGTCGAGTCCCTCAACCGCCCAATTACAGGAAGCGAGATTGTGGCCATAATTAACTCCC |
| | TGCCGACAAAGAAATCTCCTGGTCCGGACGGGTTTACAGCTGAGTTTTATCAACGGTATATGGAAGA |
| | GCTTGTACCGTTTCTGCTCAAGCTCTTTCAGTCTATAGAAAAGGAAGGCATCTTGCCCAATTCCTTC |
| | TACGAAGCTTCTATAATACTTATTCCCAAACCAGGACGCGATACCACAAAGAAGGAAAACTTCCGGC |
| | CCATTAGTCTCATGAATATCGACGCTAAAATATTGAACAAGATTCTCGCCAACAGAATCCAACAACA |
| | TATTAAGAAATTGATACATCACGACCAGGTGGGGTTTATACCTGGCATGCAGGGCTGGTTTAACATC |
| | CGGAAGAGTATTAACGTCATTCAACACATTAATAGAGCTAAGGATAAGAATCATATGATCATCTCTA |
| | TAGACGCGGAAAAGGCATTCGATAAGATTCAGCAGCCATTTATGCTCAAGACTCTGAACAAACTCGG |
| | CATCGACGGAACATATTTTAAGATTATTCGCGCAATTTACGATAAGCCGACTGCTAACATTATCCTT |
| | AACGGCCAAAAGCTCGAGGCCTTTCCGCTCAAGACTGGAACCCGCCAAGGCTGTCCCCTCTCCCCGC |
| | TTTTGTTTAATATTGTACTCGAGGTGCTGGCTAGGGCTATTCGTCAAGAGAAAGAGATTAAAGGGAT |
| | ACAGCTCGGGAAGGAAGAGGTCAAGCTTTCCTTGTTCGCCGATGATATGATTGTGTACCTGGAGAAT |
| | CCTATTGTGTCTGCTCAGAACCTTCTTAAACTTATTTCTAACTTTAGCAAGGTCAGCGGCTATAAGA |
| | TTAACGTCCAGAAATCTCAGGCCTTTCTGTACACAAATAATCGACAGACCGAATCCCAGATAATGGG |
| | TGAGCTTCCGTTTGTCATAGCCAGCAAAAGGATAAAGTATCTCGGAATCCAGCTGACACGAGACGTT |
| | AAAGATTTGTTTAAGGAAAATTACAAGCCTCTCCTGAAAGAGATTAAGGAAGATACTAATAAGTGGA |
| | AGAATATCCCCTGTTCATGGGTTGGCAGAATCAACATAGTGAAGATGGCAATACTTCCTAAAGTGAT |
| | ATATCGCTTTAACGCCATCCCAATTAAACTGCCTATGACCTTCTTTACGGAGCTCGAGAAAACAACC |
| | CTTAAATTTATATGGAATCAAAAGAGAGCAAGAATAGCGAAGTCCATCTTGAGCCAGAAGAATAAGG |
| | CCGGTGGGATTACTTTGCCTGATTTTAAGTTGTATTATAAAGCCACAGTAACTAAGACAGCCTGGTA |
| | TTGGTATCAGAATAGAGACATCGACCAGTGGAATCGGACCGAACCATCAGAGATAATGCCCCACATC |
| | TATAATTACCTTATATTCGATAAGCCAGAAAAGAATAAACAGTGGGGCAAAGACAGCCTCTTCAACA |
| | AGTGGTGTTGGGAGAATTGGCTGGCCATATGCCGGAAACTCAAGCTCGACCCCTTTCTTACACCCTA |
| | CACTAAAATCAACAGTAGGTGGATCAAGGACTTGAATGTCAAGCCAAAGACTATAAAGACACTGGAA |
| | GAGAATCTTGGGATCACAATACAAGATATAGGCGTCGGCAAAGATTTTATGTCAAAGACGCCCAAGG |
| | CCATGGCCACTAAGGATAAGATTGATAAGTGGGACCTTATTAAGCTCAAAAGCTTCTGTACTGCCAA |
| | GGAGACCACGATCAGAGTTAATAGGCAGCCCACTACATGGGAAAAGATTTTCGCCACTTATTCATCA |
| | GATAAGGGGTTGATAAGCAGAATATATAACGAGCTGAAGCAGATCTACAAGAAGAAAACGAATAATC |
| | CCATCAAGAAGTGGGCAAAAGATATGAACAGGCATTTTAGCAAAGAGGATATCTACGCCGCGAAGAA |
| | GCATATGAAGAAGTGTAGTTCAAGCTTGGCCATTCGTGAGATGCAGATTAAGACGACCATGCGATAC |
| | CACCTTACCCCAGTGAGGATGGCAATTATCAAGAAATCTGGCAATAATAGATGTTGGCGGGGCTGTG |
| | GCGAGATTGGCACCCTGCTCCATTGCTGGTGGGATTGCAAGCTGGTGCAGCCGCTTTGGAAATCAGT |
| | CTGGCGCTTTCTGAGGGACCTCGAGCTTGAGATTCCCTTCGATCCCGCAATTCCCTTGCTCGGAATC |
| | TATCCTAACGAATACAAGAGCTGTTGTTACAAGGATACGTGTACCCGGATGTTCATCGCGGCCTTGT |
| | TTACGATAGCTAAGACGTGGAATCAGCCTAAGTGCCCCACAATGATCGATTGGATCAAGAAAATGTG |
| | GCATATTTATACCATGGAGTATTACGCAGCAATTAAGAATGACGAATTTATTTCCTTCGTTGGGACC |
| | TGGATGAAGCTGGAGACTATTATTCTGAGCAAGCTGTCTCAGGAGCAAAAGACAAAGCATAGAATCT |
| | TCTCTCTCATTGGTGGTAACGACTACAAAGACGATGACGACAAGTAAAGCGCTTCTAGAAGTTGTCT |
| | CCTCCTGCACTGACTGACTGATACAATCGATTTCTGGATCCGCAGGCCTAATCAACCTCTGGATTAC |
| | AAAATTTGTGAAAGATTGACTGGTATTCTTAACTATGTTGCTCCTTTTACGCTATGTGGATACGCTG |
| | CTTTAATGCCTTTGTATCATGCTATTGCTTCCCGTATGGCTTTCATTTTCTCCTCCTTGTATAAATC |
| | CTGGTTGCTGTCTCTTTATGAGGAGTTGTGGCCCGTTGTCAGGCAACGTGGCGTGGTGTGCACTGTG |
| | TTTGCTGACGCAACCCCCACTGGTTGGGGCATTGCCACCACCTGTCAGCTCCTTTCCGGGACTTTCG |
| | CTTTCCCCCTCCCTATTGCCACGGCGGAACTCATCGCCGCCTGCCTTGCCCGCTGCTGGACAGGGGC |
| | TCGGCTGTTGGGCACTGACAATTCCGTGGTGTTGTCGGGGAAGCTGACGTCCTTTCCATGGCTGCTC |
| | GCCTGTGTTGCCACCTGGATTCTGCGCGGGACGTCCTTCTGCTACGTCCCTTCGGCCCTCAATCCAG |
| | CGGACCTTCCTTCCCGCtgagagacacaaaaaattccaacacactattgcaatgaaaataaatttcc |
| | tttattagccagaagtcagatgctcaaggggcttcatgatgtccccataatttttggcagagggaaa |
| | aagatctcagtggtatttgtgagccagggcattggccttctgataggcagcctgcacctgaggagtg |
| | cggccgctttacttgtacagctcgtccatgccgagagtgatcccggcggcggtcacgaactccagca |
| | ggaccatgtgatcgcgcttctcgttggggtctttgctcagggcggactgggtgctcaggtagtggtt |
| | gtcgggcagcagcacggggccgtcgccgatgggggtgttctgctggtagtggtcggcgagctgcacg |
| | ctgccgtcctcgatgttgtggcggatcttgaagttcaccttgatgccgttcttctgcttgtcggcca |
| | tgatatagacgttgtggctgttgtagttgtactccagcttgtgccccaggatgttgccgtcctcctt |
| | gaagtcgatgcccttcagctcgatgcggttcaccagggtgtcgccctcgaacttcacctcggcgcgg |
| | gtcttgtagttgccgtcgtccttgaagaagatggtgcgctcctggacgtagccttcgggcatggcgg |
| | acttgaagaagtcgtgctgcttcatgtggtcggggtagcggctgaagcactgcacgccgtaggtcag |
| | ggtggtcacgagggtgggccagggcacgggcagcttgccggtggtgcagatgaacttcagggtcagc |
| | ttgccgtaggtggcatcgccctcgccctcgccggacacgctgaacttgtggccgtttacgtcgccgt |
| | ccagctcgaccaggatgggcaccaccccggtgaacagctcctcgcccttgctcaccatggtggcggg |
| | atctgacggttcactaaaccagctctgcttatatagacctcccaccgtacacgcctaccgcccattt |
| | gcgtcaatggggcggagttgttacgacattttggaaagtcccgttgattttggtgccaaaacaaact |
| | cccattgacgtcaatggggtggagacttggaaatccccgtgagtcaaaccgctatccacgcccattg |
| | atgtactgccaaaaccgcatcaccatggtaatagcgatgactaatacgtagatgtactgccaagtag |
| | gaaagtcccataaggtcatgtactgggcataatgccaggcgggccatttaccgtcattgacgtcaat |
| | agggggcgtacttggcatatgatacacttgatgtactgccaagtgggcagtttaccgtaaatactcc |
| | acccattgacgtcaatggaaagtccctattggcgttactatgggaacatacgtcattattgacgtca |
| | atgggcgggggtcgttgggcggtcagccaggcgggccatttaccgtaagttatgtaacgGGCCTGCT |
| | GCCGGCTCTGCGGCCTCTTCCGCGTCTTCGCCTTCGCCCTCAGACGAGTCGGATCTCCCTTTGGGCC |
| | GCCTCCCCGCCTGTCTAGCTTGACTGACTGAGATACAGCGTACCTTCAGCTCACAGACATGATAAGA |
| | TACATTGATGAGTTTGGACAAACCACAACTAGAATGCAGTGAAAAAAATGCTTTATTTGTGAAATTT |
| | GTGATGCTATTGCTTTATTTGTAACCATTATAAGCTGCAATAAACAAGTTAACAACAACAATTGCAT |
| | TCATTTTATGTTTCAGGTTCAGGGGGAGGTGTGGGAGGTTTTTTAAAGCAAGTAAAACCTCTACAAA |
| | TGTGGTATTGGCCCATCTCTATCGGTATCGTAGCATAACCCCTTGGGGCCTCTAAACGGGTCTTGAG |
| | GGGTTTTTTGTGCCCCTCGGGCCGGATTGCTATCTACCGGCATTGGCGCAGAAAAAAATGCCTGATG |
| | CGACGCTGCGCGTCTTATACTCCCACATATGCCAGATTCAGCAACGGATACGGCTTCCCCAACTTGC |
| | CCACTTCCATACGTGTCCTCCTTACCAGAAATTTATCCTTAAGGTCGTCAGCTATCCTGCAGGCGAT |
| | CTCTCGATTTCGATCAAGACATTCCTTTAATGGTCTTTTCTGGACACCACTAGGGGTCAGAAGTAGT |
| | TCATCAAACTTTCTTCCCTCCCTAATCTCATTGGTTACCTTGGGCTATCGAAACTTAATTAAGCGAT |
| | CTGCATCTCAATTAGTCAGCAACCATAGTCCCGCCCCTAACTCCGCCCATCCCGCCCCTAACTCCGC |
| | CCAGTTCCGCCCATTCTCCGCCCCATCGCTGACTAATTTTTTTTATTTATGCAGAGGCCGAGGCCGC |
| | CTCGGCCTCTGAGCTATTCCAGAAGTAGTGAGGAGGCTTTTTTGGAGGCCTAGGCTTTTGCAAAGGA |
| | GGTAGCCAACATGATTGAACAAGATGGATTGCACGCAGGTTCTCCCGCCGCTTGGGTGGAGAGGCTA |
| | TTCGGCTATGACTGGGCACAACAGACAATCGGCTGCTCTGATGCCGCCGTGTTCCGGCTGTCAGCGC |
| | AGGGGCGCCCGGTTCTTTTTGTCAAGACCGACCTGTCCGGTGCCCTGAATGAACTCCAGGACGAGGC |
| | AGCGCGGCTATCGTGGCTGGCCACGACGGGCGTTCCTTGCGCAGCTGTGCTCGACGTTGTCACTGAA |
| | GCGGGAAGGGACTGGCTGCTATTGGGCGAAGTGCCGGGGCAGGATCTCCTGTCATCTCACCTTGCTC |
| | CTGCCGAGAAAGTATCCATCATGGCTGATGCAATGCGGCGGCTGCATACGCTTGATCCGGCTACCTG |
| | CCCATTCGACCACCAAGCGAAACATCGCATCGAGCGAGCACGTACTCGGATGGAAGCCGGTCTTGTC |
| | GATCAGGATGATCTGGACGAAGAGCATCAGGGGCTCGCGCCAGCCGAACTGTTCGCCAGGCTCAAGG |
| | CGCGGATGCCCGACGGCGAGGATCTCGTCGTGACCCACGGCGATGCCTGCTTGCCGAATATCATGGT |
| | GGAAAATGGCCGCTTTTCTGGATTCATCGACTGTGGCCGGCTGGGTGTGGCGGACCGCTATCAGGAC |
| | ATAGCGTTGGCTACCCGTGATATTGCTGAAGAGCTTGGCGGCGAATGGGCTGACCGCTTCCTCGTGC |
| | TTTACGGTATCGCCGCTCCCGATTCGCAGCGCATCGCCTTCTATCGCCTTCTTGACGAGTTCTTCTA |
| | GTATGTAAGCCCTGTGCCTTCTAGTTGCCAGCCATCTGTTGTTTGCCCCTCCCCCGTGCCTTCCTTG |
| | ACCCTGGAAGGTGCCACTCCCACTGTCCTTTCCTAATAAAATGAGGAAATTGCATCGCATTGTCTGA |
| | GTAGGTGTCATTCTATTCTGGGGGGTGGGGTGGGGCAGGACAGCAAGGGGGAGGATTGGGAAGACAA |
| | TAGCAGGCATGCTGGGGATGCGGTGGGCTCTATGGTTAATTAACCAGTCAAGTCAGCTACTTGGCGA |
| | GATCGACTTGTCTGGGTTTCGACTACGCTCAGAATTGCGTCAGTCAAGTTCGATCTGGTCCTTGCTA |
| | TTGCACCCGTTCTCCGATTACGAGTTTCATTTAAATCATGTGAGCAAAAGGCCAGCAAAAGGCCAGG |
| | AACCGTAAAAAGGCCGCGTTGCTGGCGTTTTTCCATAGGCTCCGCCCCCCTGACGAGCATCACAAAA |
| | ATCGACGCTCAAGTCAGAGGTGGCGAAACCCGACAGGACTATAAAGATACCAGGCGTTTCCCCCTGG |
| | AAGCTCCCTCGTGCGCTCTCCTGTTCCGACCCTGCCGCTTACCGGATACCTGTCCGCCTTTCTCCCT |
| | TCGGGAAGCGTGGCGCTTTCTCATAGCTCACGCTGTAGGTATCTCAGTTCGGTGTAGGTCGTTCGCT |
| | CCAAGCTGGGCTGTGTGCACGAACCCCCCGTTCAGCCCGACCGCTGCGCCTTATCCGGTAACTATCG |
| | TCTTGAGTCCAACCCGGTAAGACACGACTTATCGCCACTGGCAGCAGCCACTGGTAACAGGATTAGC |
| | AGAGCGAGGTATGTAGGCGGTGCTACAGAGTTCTTGAAGTGGTGGCCTAACTACGGCTACACTAGAA |
| | GAACAGTATTTGGTATCTGCGCTCTGCTGAAGCCAGTTACCTTCGGAAAAAGAGTTGGTAGCTCTTG |
| | ATCCGGCAAACAAACCACCGCTGGTAGCGGTGGTTTTTTTGTTTGCAAGCAGCAGATTACGCGCAGA |
| | AAAAAAGGATCTCAAGAAGATCCTTTGATCTTTTCTACGGGGTCTGACGCTCAGTGGAACGAAAACT |
| | CACGTTAAGGGATTTTGGTCATGAGATTATCAAAAAGGATCTTCACCTAGATCCTTTTAAATTAAAA |
| | ATGAAGTTTTAAATCAATCTAAAGTATATATGAGTAAACTTGGTCTGACAGTTACCAATGCTTAATC |
| | AGTGAGGCACCTATCTCAGCGATCTGTCTATTTCGTTCATCCATAGTTGCATTTAAATTTCCGAACT |
| | CTCCAAGGCCCTC (SEQ ID NO: 96) |
| RET- | GGAAAATCTTCAAACCTTTCGTCCGATCCATCTTGCAGGCTACCTCTCGAACGAACTATCGCAAGTC |
| 007 | TCTTGGCCGGCCTTGCGCCTTGGCTATTGCTTGGCAGCGCCTATCGCCAGGTATTACTCCAATCCCG |
| LINE1- | AATATCCGAGATCGGGATCACCCGAGAGAAGTTCAACCTACATCCTCAATCCCGATCTATCCGAGAT |
| GFP | CCGAGGAATATCGAAATCGGGGCGCGCCTGGTGTACCGAGAACGATCCTCTCAGTGCGAGTCTCGAC |
| ORF1- | GATCCATATCGTTGCTTGGCAGTCAGCCAGTCGGAATCCAGCTTGGGACCCAGGAAGTCCAATCGTC |
| NSV40 | AGATATTGTACTCAAGCCTGGTCACGGCAGCGTACCGATCTGTTTAAACCTAGATATTGATAGTCTG |
| NLS | ATCGGTCAACGTATAATCGAGTCCTAGCTTTTGCAAACATCTATCAAGAGACAGGATCAGCAGGAGG |
| Linker | CTTTCGCATGAGTATTCAACATTTCCGTGTCGCCCTTATTCCCTTTTTTGCGGCATTTTGCCTTCCT |
| | GTTTTTGCTCACCCAGAAACGCTGGTGAAAGTAAAAGATGCTGAAGATCAGTTGGGTGCGCGAGTGG |
| | GTTACATCGAACTGGATCTCAACAGCGGTAAGATCCTTGAGAGTTTTCGCCCCGAAGAACGCTTTCC |
| | AATGATGAGCACTTTTAAAGTTCTGCTATGTGGCGCGGTATTATCCCGTATTGACGCCGGGCAAGAG |
| | CAACTCGGTCGCCGCATACACTATTCTCAGAATGACTTGGTTGAGTATTCACCAGTCACAGAAAAGC |
| | ATCTTACGGATGGCATGACAGTAAGAGAATTATGCAGTGCTGCCATAACCATGAGTGATAACACTGC |
| | GGCCAACTTACTTCTGACAACGATTGGAGGACCGAAGGAGCTAACCGCTTTTTTGCACAACATGGGG |
| | GATCATGTAACTCGCCTTGATCGTTGGGAACCGGAGCTGAATGAAGCCATACCAAACGACGAGCGTG |
| | ACACCACGATGCCTGTAGCAATGGCAACAACCTTGCGTAAACTATTAACTGGCGAACTACTTACTCT |
| | AGCTTCCCGGCAACAGTTGATAGACTGGATGGAGGCGGATAAAGTTGCAGGACCACTTCTGCGCTCG |
| | GCCCTTCCGGCTGGCTGGTTTATTGCTGATAAATCTGGAGCCGGTGAGCGTGGGTCTCGCGGTATCA |
| | TTGCAGCACTGGGGCCAGATGGTAAGCCCTCCCGTATCGTAGTTATCTACACGACGGGGAGTCAGGC |
| | AACTATGGATGAACGAAATAGACAGATCGCTGAGATAGGTGCCTCACTGATTAAGCATTGGTAACCG |
| | ATTCTAGGTGCATTGGCGCAGAAAAAAATGCCTGATGCGACGCTGCGCGTCTTATACTCCCACATAT |
| | GCCAGATTCAGCAACGGATACGGCTTCCCCAACTTGCCCACTTCCATACGTGTCCTCCTTACCAGAA |
| | ATTTATCCTTAAGATCGTTTAAACTCGACTCTGGCTCTATCGAATCTCCGTCGTTTCGAGCTTACGC |
| | GAACAGCCGTGGCGCTCATTTGCTCGTCGGGCATCGAATCTCGTCAGCTATCGTCAGCTTACCTTTT |
| | TGGCAGCGATCGCGGCTCCCGACATCTTGGACCATTAGCTCCACAGGTATCTTCTTCCCTCTAGTGG |
| | TCATAACAGCAGCTTCAGCTACCTCTCAATTCAAAAAACCCCTCAAGACCCGTTTAGAGGCCCCAAG |
| | GGGTTATGCTATCAATCGTTGCGTTACACACACAAAAAACCAACACACATCCATCTTCGATGGATAG |
| | CGATTTTATTATCTAACTGCTGATCGAGTGTAGCCAGATCTAGTAATCAATTACGGGGTCATTAGTT |
| | CATAGCCCATATATGGAGTTCCGCGTTACATAACTTACGGTAAATGGCCCGCCTGGCTGACCGCCCA |
| | ACGACCCCCGCCCATTGACGTCAATAATGACGTATGTTCCCATAGTAACGCCAATAGGGACTTTCCA |
| | TTGACGTCAATGGGTGGAGTATTTACGGTAAACTGCCCACTTGGCAGTACATCAAGTGTATCATATG |
| | CCAAGTACGCCCCCTATTGACGTCAATGACGGTAAATGGCCCGCCTGGCATTATGCCCAGTACATGA |
| | CCTTATGGGACTTTCCTACTTGGCAGTACATCTACGTATTAGTCATCGCTATTACCATGCTGATGCG |
| | GTTTTGGCAGTACATCAATGGGCGTGGATAGCGGTTTGACTCACGGGGATTTCCAAGTCTCCACCCC |
| | ATTGACGTCAATGGGAGTTTGTTTTGGCACCAAAATCAACGGGACTTTCCAAAATGTCGTAACAACT |
| | CCGCCCCATTGACGCAAATGGGCGGTAGGCGTGTACGGTGGGAGGTCTATATAAGCAGAGCTGGTTT |
| | AGTGAACCGTCAGATCAGATCTTTGTCGATCCTACCATCCACTCGACACACCCGCCAGCGGCCGCTA |
| | ATACGACTCACTATAGGGAGAAGTACTGCCACCATGccaaagaagaagcggaaggtcggcggcggca |
| | gcGGCAAGAAGCAAAATCGCAAGACGGGGAATTCCAAGACACAATCCGCTAGCCCACCACCTAAAGA |
| | GCGTTCTAGCTCCCCTGCTACTGAGCAGTCCTGGATGGAAAACGACTTCGATGAACTCCGGGAAGAG |
| | GGATTTAGGCGATCCAACTATTCAGAACTCCGCGAAGATATCCAGACAAAGGGGAAGGAAGTCGAGA |
| | ATTTCGAGAAGAACCTCGAGGAGTGCATCACCCGTATCACAAACACTGAGAAATGTCTCAAAGAACT |
| | CATGGAACTTAAGACAAAAGCCAGGGAGCTTCGAGAGGAGTGTCGGAGTCTGAGATCCAGGTGTGAC |
| | CAGCTCGAGGAGCGCGTGAGCGCGATGGAAGACGAGATGAACGAGATGAAAAGAGAGGGCAAATTCA |
| | GGGAGAAGCGCATTAAGAGGAACGAACAGAGTCTGCAGGAGATTTGGGATTACGTCAAGAGGCCTAA |
| | CCTGCGGTTGATCGGCGTCCCCGAGAGCGACGTAGAAAACGGGACTAAACTGGAGAATACACTTCAA |
| | GACATCATTCAAGAAAATTTTCCAAACCTGGCTCGGCAAGCTAATGTGCAAATCCAAGAGATCCAAC |
| | GCACACCCCAGCGGTATAGCTCTCGGCGTGCCACCCCTAGGCATATTATCGTGCGCTTTACTAAGGT |
| | GGAGATGAAAGAGAAGATGCTGCGAGCCGCTCGGGAAAAGGGAAGGGTGACTTTGAAGGGCAAACCT |
| | ATTCGGCTGACGGTTGACCTTAGCGCCGAGACACTCCAGGCACGCCGGGAATGGGGCCCCATCTTTA |
| | ATATCCTGAAGGAGAAGAACTTCCAGCCACGAATCTCTTACCCTGCAAAGTTGAGTTTTATCTCCGA |
| | GGGTGAGATTAAGTATTTCATCGATAAACAGATGCTGCGAGACTTCGTGACAACTCGCCCAGCTCTC |
| | AAGGAACTGCTCAAAGAGGCTCTTAATATGGAGCGCAATAATAGATATCAACCCTTGCAGAACCACG |
| | CAAAGATGTGAGACAGCCGTCAGACCATCAAGACTAGGAAGAAACTGCATCAACTAATGAGCAAAAT |
| | CACCAGCTAACATCATAGTATACATGACCGGCTCTAACTCACATATCACCATCCTTACACTTAACAT |
| | TAACGGCCTCAACTCAGCTATCAAGCGCCATCGGCTGGCCAGCTGGATCAAATCACAGGATCCAAGC |
| | GTTTGTTGCATCCAAGAGACCCACCTGACCTGTAGAGATACTCACCGCCTCAAGATCAAGGGATGGC |
| | GAAAGATTTATCAGGCGAACGGTAAGCAGAAGAAAGCCGGAGTCGCAATTCTGGTCTCAGACAAGAC |
| | GGATTTCAAGCCCACCAAAATTAAGCGTGATAAGGAAGGTCACTATATTATGGTGAAAGGCAGCATA |
| | CAGCAGGAAGAACTTACCATATTGAACATCTACGCGCCAAACACCGGCGCACCTCGCTTTATCAAAC |
| | AGGTCCTGTCCGATCTGCAGCGAGATCTGGATTCTCATACGTTGATTATGGGTGATTTCAATACACC |
| | ATTGAGCACCCTGGATCGCAGCACCAGGCAAAAGGTAAATAAAGACACGCAAGAGCTCAATAGCGCA |
| | CTGCATCAGGCAGATCTCATTGATATTTATCGCACTCTTCATCCTAAGAGTACCGAGTACACATTCT |
| | TCAGCGCCCCACATCATACATACTCAAAGATCGATCATATCGTCGGCTCAAAGGCTCTGCTGTCAAA |
| | GTGCAAGCGCACAGAGATAATTACAAATTACCTGTCAGATCATAGCGCGATCAAGCTCGAGCTGAGA |
| | ATCAAGAACCTGACCCAGAGCCGGAGTACCACTTGGAAGCTTAATAACCTGCTGCTCAACGATTATT |
| | GGGTCCACAATGAGATGAAGGCAGAGATTAAAATGTTCTTCGAAACAAATGAGAATAAGGATACTAC |
| | CTATCAAAACCTTTGGGATGCCTTTAAGGCCGTCTGCAGAGGCAAGTTCATCGCCCTCAACGCCTAT |
| | AAAAGAAAACAAGAGAGATCTAAGATCGATACTCTCACCTCTCAGCTGAAGGAGTTGGAGAAACAGG |
| | AACAGACCCACTCCAAGGCGTCAAGACGGCAGGAGATCACAAAGATTCGCGCCGAGTTGAAAGAGAT |
| | CGAAACCCAAAAGACTCTTCAGAAAATTAACGAGTCTCGTAGTTGGTTCTTCGAGCGGATTAATAAG |
| | ATAGACAGACCTCTGGCACGACTGATTAAGAAGAAGCGCGAAAAGAACCAGATTGATACCATCAAGA |
| | ACGACAAGGGCGACATCACTACTGACCCGACCGAGATCCAGACCACTATTCGGGAGTATTATAAGCA |
| | TTTGTATGCTAACAAGCTTGAGAACCTGGAAGAGATGGACACTTTTCTGGATACCTATACTCTGCCA |
| | CGGCTTAATCAAGAGGAAGTCGAGTCCCTCAACCGCCCAATTACAGGAAGCGAGATTGTGGCCATAA |
| | TTAACTCCCTGCCGACAAAGAAATCTCCTGGTCCGGACGGGTTTACAGCTGAGTTTTATCAACGGTA |
| | TATGGAAGAGCTTGTACCGTTTCTGCTCAAGCTCTTTCAGTCTATAGAAAAGGAAGGCATCTTGCCC |
| | AATTCCTTCTACGAAGCTTCTATAATACTTATTCCCAAACCAGGACGCGATACCACAAAGAAGGAAA |
| | ACTTCCGGCCCATTAGTCTCATGAATATCGACGCTAAAATATTGAACAAGATTCTCGCCAACAGAAT |
| | CCAACAACATATTAAGAAATTGATACATCACGACCAGGTGGGGTTTATACCTGGCATGCAGGGCTGG |
| | TTTAACATCCGGAAGAGTATTAACGTCATTCAACACATTAATAGAGCTAAGGATAAGAATCATATGA |
| | TCATCTCTATAGACGCGGAAAAGGCATTCGATAAGATTCAGCAGCCATTTATGCTCAAGACTCTGAA |
| | CAAACTCGGCATCGACGGAACATATTTTAAGATTATTCGCGCAATTTACGATAAGCCGACTGCTAAC |
| | ATTATCCTTAACGGCCAAAAGCTCGAGGCCTTTCCGCTCAAGACTGGAACCCGCCAAGGCTGTCCCC |
| | TCTCCCCGCTTTTGTTTAATATTGTACTCGAGGTGCTGGCTAGGGCTATTCGTCAAGAGAAAGAGAT |
| | TAAAGGGATACAGCTCGGGAAGGAAGAGGTCAAGCTTTCCTTGTTCGCCGATGATATGATTGTGTAC |
| | CTGGAGAATCCTATTGTGTCTGCTCAGAACCTTCTTAAACTTATTTCTAACTTTAGCAAGGTCAGCG |
| | GCTATAAGATTAACGTCCAGAAATCTCAGGCCTTTCTGTACACAAATAATCGAGAGACCGAATCCCA |
| | GATAATGGGTGAGCTTCCGTTTGTCATAGCCAGCAAAAGGATAAAGTATCTCGGAATCCAGCTGACA |
| | CGAGACGTTAAAGATTTGTTTAAGGAAAATTACAAGCCTCTCCTGAAAGAGATTAAGGAAGATACTA |
| | ATAAGTGGAAGAATATCCCCTGTTCATGGGTTGGCAGAATCAACATAGTGAAGATGGCAATACTTCC |
| | TAAAGTGATATATCGCTTTAACGCCATCCCAATTAAACTGCCTATGACCTTCTTTACGGAGCTCGAG |
| | AAAACAACCCTTAAATTTATATGGAATCAAAAGAGAGCAAGAATAGCGAAGTCCATCTTGAGCCAGA |
| | AGAATAAGGCCGGTGGGATTACTTTGCCTGATTTTAAGTTGTATTATAAAGCCACAGTAACTAAGAC |
| | AGCCTGGTATTGGTATCAGAATAGAGACATCGACCAGTGGAATCGGACCGAACCATCAGAGATAATG |
| | CCCCACATCTATAATTACCTTATATTCGATAAGCCAGAAAAGAATAAACAGTGGGGCAAAGACAGCC |
| | TCTTCAACAAGTGGTGTTGGGAGAATTGGCTGGCCATATGCCGGAAACTCAAGCTCGACCCCTTTCT |
| | TACACCCTACACTAAAATCAACAGTAGGTGGATCAAGGACTTGAATGTCAAGCCAAAGACTATAAAG |
| | ACACTGGAAGAGAATCTTGGGATCACAATACAAGATATAGGCGTCGGCAAAGATTTTATGTCAAAGA |
| | CGCCCAAGGCCATGGCCACTAAGGATAAGATTGATAAGTGGGACCTTATTAAGCTCAAAAGCTTCTG |
| | TACTGCCAAGGAGACCACGATCAGAGTTAATAGGCAGCCCACTACATGGGAAAAGATTTTCGCCACT |
| | TATTCATCAGATAAGGGGTTGATAAGCAGAATATATAACGAGCTGAAGCAGATCTACAAGAAGAAAA |
| | CGAATAATCCCATCAAGAAGTGGGCAAAAGATATGAACAGGCATTTTAGCAAAGAGGATATCTACGC |
| | CGCGAAGAAGCATATGAAGAAGTGTAGTTCAAGCTTGGCCATTCGTGAGATGCAGATTAAGACGACC |
| | ATGCGATACCACCTTACCCCAGTGAGGATGGCAATTATCAAGAAATCTGGCAATAATAGATGTTGGC |
| | GGGGCTGTGGCGAGATTGGCACCCTGCTCCATTGCTGGTGGGATTGCAAGCTGGTGCAGCCGCTTTG |
| | GAAATCAGTCTGGCGCTTTCTGAGGGACCTCGAGCTTGAGATTCCCTTCGATCCCGCAATTCCCTTG |
| | CTCGGAATCTATCCTAACGAATACAAGAGCTGTTGTTACAAGGATACGTGTACCCGGATGTTCATCG |
| | CGGCCTTGTTTACGATAGCTAAGACGTGGAATCAGCCTAAGTGCCCCACAATGATCGATTGGATCAA |
| | GAAAATGTGGCATATTTATACCATGGAGTATTACGCAGCAATTAAGAATGACGAATTTATTTCCTTC |
| | GTTGGGACCTGGATGAAGCTGGAGACTATTATTCTGAGCAAGCTGTCTCAGGAGCAAAAGACAAAGC |
| | ATAGAATCTTCTCTCTCATTGGTGGTAACGACTACAAAGACGATGACGACAAGTAAAGCGCTTCTAG |
| | AAGTTGTCTCCTCCTGCACTGACTGACTGATACAATCGATTTCTGGATCCGCAGGCCTAATCAACCT |
| | CTGGATTACAAAATTTGTGAAAGATTGACTGGTATTCTTAACTATGTTGCTCCTTTTACGCTATGTG |
| | GATACGCTGCTTTAATGCCTTTGTATCATGCTATTGCTTCCCGTATGGCTTTCATTTTCTCCTCCTT |
| | GTATAAATCCTGGTTGCTGTCTCTTTATGAGGAGTTGTGGCCCGTTGTCAGGCAACGTGGCGTGGTG |
| | TGCACTGTGTTTGCTGACGCAACCCCCACTGGTTGGGGCATTGCCACCACCTGTCAGCTCCTTTCCG |
| | GGACTTTCGCTTTCCCCCTCCCTATTGCCACGGCGGAACTCATCGCCGCCTGCCTTGCCCGCTGCTG |
| | GACAGGGGCTCGGCTGTTGGGCACTGACAATTCCGTGGTGTTGTCGGGGAAGCTGACGTCCTTTCCA |
| | TGGCTGCTCGCCTGTGTTGCCACCTGGATTCTGCGCGGGACGTCCTTCTGCTACGTCCCTTCGGCCC |
| | TCAATCCAGCGGACCTTCCTTCCCGCtgagagacacaaaaaattccaacacactattgcaatgaaaa |
| | taaatttcctttattagccagaagtcagatgctcaaggggcttcatgatgtccccataatttttggc |
| | agagggaaaaagatctcagtggtatttgtgagccagggcattggccttctgataggcagcctgcacc |
| | tgaggagtgcggccgctttacttgtacagctcgtccatgccgagagtgatcccggcggcggtcacga |
| | actccagcaggaccatgtgatcgcgcttctcgttggggtctttgctcagggcggactgggtgctcag |
| | gtagtggttgtcgggcagcagcacggggccgtcgccgatgggggtgttctgctggtagtggtcggcg |
| | agctgcacgctgccgtcctcgatgttgtggcggatcttgaagttcaccttgatgccgttcttctgct |
| | tgtcggccatgatatagacgttgtggctgttgtagttgtactccagcttgtgccccaggatgttgcc |
| | gtcctccttgaagtcgatgcccttcagctcgatgcggttcaccagggtgtcgccctcgaacttcacc |
| | tcggcgcgggtcttgtagttgccgtcgtccttgaagaagatggtgcgctcctggacgtagccttcgg |
| | gcatggcggacttgaagaagtcgtgctgcttcatgtggtcggggtagcggctgaagcactgcacgcc |
| | gtaggtcagggtggtcacgagggtgggccagggcacgggcagcttgccggtggtgcagatgaacttc |
| | agggtcagcttgccgtaggtggcatcgccctcgccctcgccggacacgctgaacttgtggccgttta |
| | cgtcgccgtccagctcgaccaggatgggcaccaccccggtgaacagctcctcgcccttgctcaccat |
| | ggtggcgggatctgacggttcactaaaccagctctgcttatatagacctcccaccgtacacgcctac |
| | cgcccatttgcgtcaatggggcggagttgttacgacattttggaaagtcccgttgattttggtgcca |
| | aaacaaactcccattgacgtcaatggggtggagacttggaaatccccgtgagtcaaaccgctatcca |
| | cgcccattgatgtactgccaaaaccgcatcaccatggtaatagcgatgactaatacgtagatgtact |
| | gccaagtaggaaagtcccataaggtcatgtactgggcataatgccaggcgggccatttaccgtcatt |
| | gacgtcaatagggggcgtacttggcatatgatacacttgatgtactgccaagtgggcagtttaccgt |
| | aaatactccacccattgacgtcaatggaaagtccctattggcgttactatgggaacatacgtcatta |
| | ttgacgtcaatgggcgggggtcgttgggcggtcagccaggcgggccatttaccgtaagttatgtaac |
| | gGGCCTGCTGCCGGCTCTGCGGCCTCTTCCGCGTCTTCGCCTTCGCCCTCAGACGAGTCGGATCTCC |
| | CTTTGGGCCGCCTCCCCGCCTGTCTAGCTTGACTGACTGAGATACAGCGTACCTTCAGCTCACAGAC |
| | ATGATAAGATACATTGATGAGTTTGGACAAACCACAACTAGAATGCAGTGAAAAAAATGCTTTATTT |
| | GTGAAATTTGTGATGCTATTGCTTTATTTGTAACCATTATAAGCTGCAATAAACAAGTTAACAACAA |
| | CAATTGCATTCATTTTATGTTTCAGGTTCAGGGGGAGGTGTGGGAGGTTTTTTAAAGCAAGTAAAAC |
| | CTCTACAAATGTGGTATTGGCCCATCTCTATCGGTATCGTAGCATAACCCCTTGGGGCCTCTAAACG |
| | GGTCTTGAGGGGTTTTTTGTGCCCCTCGGGCCGGATTGCTATCTACCGGCATTGGCGCAGAAAAAAA |
| | TGCCTGATGCGACGCTGCGCGTCTTATACTCCCACATATGCCAGATTCAGCAACGGATACGGCTTCC |
| | CCAACTTGCCCACTTCCATACGTGTCCTCCTTACCAGAAATTTATCCTTAAGGTCGTCAGCTATCCT |
| | GCAGGCGATCTCTCGATTTCGATCAAGACATTCCTTTAATGGTCTTTTCTGGACACCACTAGGGGTC |
| | AGAAGTAGTTCATCAAACTTTCTTCCCTCCCTAATCTCATTGGTTACCTTGGGCTATCGAAACTTAA |
| | TTAAGCGATCTGCATCTCAATTAGTCAGCAACCATAGTCCCGCCCCTAACTCCGCCCATCCCGCCCC |
| | TAACTCCGCCCAGTTCCGCCCATTCTCCGCCCCATCGCTGACTAATTTTTTTTATTTATGCAGAGGC |
| | CGAGGCCGCCTCGGCCTCTGAGCTATTCCAGAAGTAGTGAGGAGGCTTTTTTGGAGGCCTAGGCTTT |
| | TGCAAAGGAGGTAGCCAACATGATTGAACAAGATGGATTGCACGCAGGTTCTCCCGCCGCTTGGGTG |
| | GAGAGGCTATTCGGCTATGACTGGGCACAACAGACAATCGGCTGCTCTGATGCCGCCGTGTTCCGGC |
| | TGTCAGCGCAGGGGCGCCCGGTTCTTTTTGTCAAGACCGACCTGTCCGGTGCCCTGAATGAACTCCA |
| | GGACGAGGCAGCGCGGCTATCGTGGCTGGCCACGACGGGCGTTCCTTGCGCAGCTGTGCTCGACGTT |
| | GTCACTGAAGCGGGAAGGGACTGGCTGCTATTGGGCGAAGTGCCGGGGCAGGATCTCCTGTCATCTC |
| | ACCTTGCTCCTGCCGAGAAAGTATCCATCATGGCTGATGCAATGCGGCGGCTGCATACGCTTGATCC |
| | GGCTACCTGCCCATTCGACCACCAAGCGAAACATCGCATCGAGCGAGCACGTACTCGGATGGAAGCC |
| | GGTCTTGTCGATCAGGATGATCTGGACGAAGAGCATCAGGGGCTCGCGCCAGCCGAACTGTTCGCCA |
| | GGCTCAAGGCGCGGATGCCCGACGGCGAGGATCTCGTCGTGACCCACGGCGATGCCTGCTTGCCGAA |
| | TATCATGGTGGAAAATGGCCGCTTTTCTGGATTCATCGACTGTGGCCGGCTGGGTGTGGCGGACCGC |
| | TATCAGGACATAGCGTTGGCTACCCGTGATATTGCTGAAGAGCTTGGCGGCGAATGGGCTGACCGCT |
| | TCCTCGTGCTTTACGGTATCGCCGCTCCCGATTCGCAGCGCATCGCCTTCTATCGCCTTCTTGACGA |
| | GTTCTTCTAGTATGTAAGCCCTGTGCCTTCTAGTTGCCAGCCATCTGTTGTTTGCCCCTCCCCCGTG |
| | CCTTCCTTGACCCTGGAAGGTGCCACTCCCACTGTCCTTTCCTAATAAAATGAGGAAATTGCATCGC |
| | ATTGTCTGAGTAGGTGTCATTCTATTCTGGGGGGTGGGGTGGGGCAGGACAGCAAGGGGGAGGATTG |
| | GGAAGACAATAGCAGGCATGCTGGGGATGCGGTGGGCTCTATGGTTAATTAACCAGTCAAGTCAGCT |
| | ACTTGGCGAGATCGACTTGTCTGGGTTTCGACTACGCTCAGAATTGCGTCAGTCAAGTTCGATCTGG |
| | TCCTTGCTATTGCACCCGTTCTCCGATTACGAGTTTCATTTAAATCATGTGAGCAAAAGGCCAGCAA |
| | AAGGCCAGGAACCGTAAAAAGGCCGCGTTGCTGGCGTTTTTCCATAGGCTCCGCCCCCCTGACGAGC |
| | ATCACAAAAATCGAGGCTCAAGTCAGAGGTGGCGAAACCCGACAGGACTATAAAGATACCAGGCGTT |
| | TCCCCCTGGAAGCTCCCTCGTGCGCTCTCCTGTTCCGACCCTGCCGCTTACCGGATACCTGTCCGCC |
| | TTTCTCCCTTCGGGAAGCGTGGCGCTTTCTCATAGCTCACGCTGTAGGTATCTCAGTTCGGTGTAGG |
| | TCGTTCGCTCCAAGCTGGGCTGTGTGCACGAACCCCCCGTTCAGCCCGACCGCTGCGCCTTATCCGG |
| | TAACTATCGTCTTGAGTCCAACCCGGTAAGACACGACTTATCGCCACTGGCAGCAGCCACTGGTAAC |
| | AGGATTAGCAGAGCGAGGTATGTAGGCGGTGCTACAGAGTTCTTGAAGTGGTGGCCTAACTACGGCT |
| | ACACTAGAAGAACAGTATTTGGTATCTGCGCTCTGCTGAAGCCAGTTACCTTCGGAAAAAGAGTTGG |
| | TAGCTCTTGATCCGGCAAACAAACCACCGCTGGTAGCGGTGGTTTTTTTGTTTGCAAGCAGCAGATT |
| | ACGCGCAGAAAAAAAGGATCTCAAGAAGATCCTTTGATCTTTTCTACGGGGTCTGACGCTCAGTGGA |
| | ACGAAAACTCACGTTAAGGGATTTTGGTCATGAGATTATCAAAAAGGATCTTCACCTAGATCCTTTT |
| | AAATTAAAAATGAAGTTTTAAATCAATCTAAAGTATATATGAGTAAACTTGGTCTGACAGTTACCAA |
| | TGCTTAATCAGTGAGGCACCTATCTCAGCGATCTGTCTATTTCGTTCATCCATAGTTGCATTTAAAT |
| | TTCCGAACTCTCCAAGGCCCTCGTC (SEQ ID NO: 97) |
| RET- | GGAAAATCTTCAAACCTTTCGTCCGATCCATCTTGCAGGCTACCTCTCGAACGAACTATCGCAAGTC |
| 008 | TCTTGGCCGGCCTTGCGCCTTGGCTATTGCTTGGCAGCGCCTATCGCCAGGTATTACTCCAATCCCG |
| LINE1- | AATATCCGAGATCGGGATCACCCGAGAGAAGTTCAACCTACATCCTCAATCCCGATCTATCCGAGAT |
| GFP | CCGAGGAATATCGAAATCGGGGCGCGCCTGGTGTACCGAGAACGATCCTCTCAGTGCGAGTCTCGAC |
| ORF1-N | GATCCATATCGTTGCTTGGCAGTCAGCCAGTCGGAATCCAGCTTGGGACCCAGGAAGTCCAATCGTC |
| Nucleo- | AGATATTGTACTCAAGCCTGGTCACGGCAGCGTACCGATCTGTTTAAACCTAGATATTGATAGTCTG |
| plasmin | ATCGGTCAACGTATAATCGAGTCCTAGCTTTTGCAAACATCTATCAAGAGACAGGATCAGCAGGAGG |
| NLS | CTTTCGCATGAGTATTCAACATTTCCGTGTCGCCCTTATTCCCTTTTTTGCGGCATTTTGCCTTCCT |
| | GTTTTTGCTCACCCAGAAACGCTGGTGAAAGTAAAAGATGCTGAAGATCAGTTGGGTGCGCGAGTGG |
| | GTTACATCGAACTGGATCTCAACAGCGGTAAGATCCTTGAGAGTTTTCGCCCCGAAGAACGCTTTCC |
| | AATGATGAGCACTTTTAAAGTTCTGCTATGTGGCGCGGTATTATCCCGTATTGACGCCGGGCAAGAG |
| | CAACTCGGTCGCCGCATACACTATTCTCAGAATGACTTGGTTGAGTATTCACCAGTCACAGAAAAGC |
| | ATCTTACGGATGGCATGACAGTAAGAGAATTATGCAGTGCTGCCATAACCATGAGTGATAACACTGC |
| | GGCCAACTTACTTCTGACAACGATTGGAGGACCGAAGGAGCTAACCGCTTTTTTGCACAACATGGGG |
| | GATCATGTAACTCGCCTTGATCGTTGGGAACCGGAGCTGAATGAAGCCATACCAAACGACGAGCGTG |
| | ACACCACGATGCCTGTAGCAATGGCAACAACCTTGCGTAAACTATTAACTGGCGAACTACTTACTCT |
| | AGCTTCCCGGCAACAGTTGATAGACTGGATGGAGGCGGATAAAGTTGCAGGACCACTTCTGCGCTCG |
| | GCCCTTCCGGCTGGCTGGTTTATTGCTGATAAATCTGGAGCCGGTGAGCGTGGGTCTCGCGGTATCA |
| | TTGCAGCACTGGGGCCAGATGGTAAGCCCTCCCGTATCGTAGTTATCTACACGACGGGGAGTCAGGC |
| | AACTATGGATGAACGAAATAGACAGATCGCTGAGATAGGTGCCTCACTGATTAAGCATTGGTAACCG |
| | ATTCTAGGTGCATTGGCGCAGAAAAAAATGCCTGATGCGACGCTGCGCGTCTTATACTCCCACATAT |
| | GCCAGATTCAGCAACGGATACGGCTTCCCCAACTTGCCCACTTCCATACGTGTCCTCCTTACCAGAA |
| | ATTTATCCTTAAGATCGTTTAAACTCGACTCTGGCTCTATCGAATCTCCGTCGTTTCGAGCTTACGC |
| | GAACAGCCGTGGCGCTCATTTGCTCGTCGGGCATCGAATCTCGTCAGCTATCGTCAGCTTACCTTTT |
| | TGGCAGCGATCGCGGCTCCCGACATCTTGGACCATTAGCTCCACAGGTATCTTCTTCCCTCTAGTGG |
| | TCATAACAGCAGCTTCAGCTACCTCTCAATTCAAAAAACCCCTCAAGACCCGTTTAGAGGCCCCAAG |
| | GGGTTATGCTATCAATCGTTGCGTTACACACACAAAAAACCAACACACATCCATCTTCGATGGATAG |
| | CGATTTTATTATCTAACTGCTGATCGAGTGTAGCCAGATCTAGTAATCAATTACGGGGTCATTAGTT |
| | CATAGCCCATATATGGAGTTCCGCGTTACATAACTTACGGTAAATGGCCCGCCTGGCTGACCGCCCA |
| | ACGACCCCCGCCCATTGACGTCAATAATGACGTATGTTCCCATAGTAACGCCAATAGGGACTTTCCA |
| | TTGACGTCAATGGGTGGAGTATTTACGGTAAACTGCCCACTTGGCAGTACATCAAGTGTATCATATG |
| | CCAAGTACGCCCCCTATTGACGTCAATGACGGTAAATGGCCCGCCTGGCATTATGCCCAGTACATGA |
| | CCTTATGGGACTTTCCTACTTGGCAGTACATCTACGTATTAGTCATCGCTATTACCATGCTGATGCG |
| | GTTTTGGCAGTACATCAATGGGCGTGGATAGCGGTTTGACTCACGGGGATTTCCAAGTCTCCACCCC |
| | ATTGACGTCAATGGGAGTTTGTTTTGGCACCAAAATCAACGGGACTTTCCAAAATGTCGTAACAACT |
| | CCGCCCCATTGACGCAAATGGGCGGTAGGCGTGTACGGTGGGAGGTCTATATAAGCAGAGCTGGTTT |
| | AGTGAACCGTCAGATCAGATCTTTGTCGATCCTACCATCCACTCGACACACCCGCCAGCGGCCGCTA |
| | ATACGACTCACTATAGGGAGAAGTACTGCCACCATGaaaaggccggcggccacgaaaaaggccggcc |
| | aggcaaaaaagaaaaagGGCAAGAAGCAAAATCGCAAGACGGGGAATTCCAAGACACAATCCGCTAG |
| | CCCACCACCTAAAGAGCGTTCTAGCTCCCCTGCTACTGAGCAGTCCTGGATGGAAAACGACTTCGAT |
| | GAACTCCGGGAAGAGGGATTTAGGCGATCCAACTATTCAGAACTCCGCGAAGATATCCAGACAAAGG |
| | GGAAGGAAGTCGAGAATTTCGAGAAGAACCTCGAGGAGTGCATCACCCGTATCACAAACACTGAGAA |
| | ATGTCTCAAAGAACTCATGGAACTTAAGACAAAAGCCAGGGAGCTTCGAGAGGAGTGTCGGAGTCTG |
| | AGATCCAGGTGTGACCAGCTCGAGGAGCGCGTGAGCGCGATGGAAGACGAGATGAACGAGATGAAAA |
| | GAGAGGGCAAATTCAGGGAGAAGCGCATTAAGAGGAACGAACAGAGTCTGCAGGAGATTTGGGATTA |
| | CGTCAAGAGGCCTAACCTGCGGTTGATCGGCGTCCCCGAGAGCGACGTAGAAAACGGGACTAAACTG |
| | GAGAATACACTTCAAGACATCATTCAAGAAAATTTTCCAAACCTGGCTCGGCAAGCTAATGTGCAAA |
| | TCCAAGAGATCCAACGCACACCCCAGCGGTATAGCTCTCGGCGTGCCACCCCTAGGCATATTATCGT |
| | GCGCTTTACTAAGGTGGAGATGAAAGAGAAGATGCTGCGAGCCGCTCGGGAAAAGGGAAGGGTGACT |
| | TTGAAGGGCAAACCTATTCGGCTGACGGTTGACCTTAGCGCCGAGACACTCCAGGCACGCCGGGAAT |
| | GGGGCCCCATCTTTAATATCCTGAAGGAGAAGAACTTCCAGCCACGAATCTCTTACCCTGCAAAGTT |
| | GAGTTTTATCTCCGAGGGTGAGATTAAGTATTTCATCGATAAACAGATGCTGCGAGACTTCGTGACA |
| | ACTCGCCCAGCTCTCAAGGAACTGCTCAAAGAGGCTCTTAATATGGAGCGCAATAATAGATATCAAC |
| | CCTTGCAGAACCACGCAAAGATGTGAGACAGCCGTCAGACCATCAAGACTAGGAAGAAACTGCATCA |
| | ACTAATGAGCAAAATCACCAGCTAACATCATAGTATACATGACCGGCTCTAACTCACATATCACCAT |
| | CCTTACACTTAACATTAACGGCCTCAACTCAGCTATCAAGCGCCATCGGCTGGCCAGCTGGATCAAA |
| | TCACAGGATCCAAGCGTTTGTTGCATCCAAGAGACCCACCTGACCTGTAGAGATACTCACCGCCTCA |
| | AGATCAAGGGATGGCGAAAGATTTATCAGGCGAACGGTAAGCAGAAGAAAGCCGGAGTCGCAATTCT |
| | GGTCTCAGACAAGACGGATTTCAAGCCCACCAAAATTAAGCGTGATAAGGAAGGTCACTATATTATG |
| | GTGAAAGGCAGCATACAGCAGGAAGAACTTACCATATTGAACATCTACGCGCCAAACACCGGCGCAC |
| | CTCGCTTTATCAAACAGGTCCTGTCCGATCTGCAGCGAGATCTGGATTCTCATACGTTGATTATGGG |
| | TGATTTCAATACACCATTGAGCACCCTGGATCGCAGCACCAGGCAAAAGGTAAATAAAGACACGCAA |
| | GAGCTCAATAGCGCACTGCATCAGGCAGATCTCATTGATATTTATCGCACTCTTCATCCTAAGAGTA |
| | CCGAGTACACATTCTTCAGCGCCCCACATCATACATACTCAAAGATCGATCATATCGTCGGCTCAAA |
| | GGCTCTGCTGTCAAAGTGCAAGCGCACAGAGATAATTACAAATTACCTGTCAGATCATAGCGCGATC |
| | AAGCTCGAGCTGAGAATCAAGAACCTGACCCAGAGCCGGAGTACCACTTGGAAGCTTAATAACCTGC |
| | TGCTCAACGATTATTGGGTCCACAATGAGATGAAGGCAGAGATTAAAATGTTCTTCGAAACAAATGA |
| | GAATAAGGATACTACCTATCAAAACCTTTGGGATGCCTTTAAGGCCGTCTGCAGAGGCAAGTTCATC |
| | GCCCTCAACGCCTATAAAAGAAAACAAGAGAGATCTAAGATCGATACTCTCACCTCTCAGCTGAAGG |
| | AGTTGGAGAAACAGGAACAGACCCACTCCAAGGCGTCAAGACGGCAGGAGATCACAAAGATTCGCGC |
| | CGAGTTGAAAGAGATCGAAACCCAAAAGACTCTTCAGAAAATTAACGAGTCTCGTAGTTGGTTCTTC |
| | GAGCGGATTAATAAGATAGACAGACCTCTGGCACGACTGATTAAGAAGAAGCGCGAAAAGAACCAGA |
| | TTGATACCATCAAGAACGACAAGGGCGACATCACTACTGACCCGACCGAGATCCAGACCACTATTCG |
| | GGAGTATTATAAGCATTTGTATGCTAACAAGCTTGAGAACCTGGAAGAGATGGACACTTTTCTGGAT |
| | ACCTATACTCTGCCACGGCTTAATCAAGAGGAAGTCGAGTCCCTCAACCGCCCAATTACAGGAAGCG |
| | AGATTGTGGCCATAATTAACTCCCTGCCGACAAAGAAATCTCCTGGTCCGGACGGGTTTACAGCTGA |
| | GTTTTATCAACGGTATATGGAAGAGCTTGTACCGTTTCTGCTCAAGCTCTTTCAGTCTATAGAAAAG |
| | GAAGGCATCTTGCCCAATTCCTTCTACGAAGCTTCTATAATACTTATTCCCAAACCAGGACGCGATA |
| | CCACAAAGAAGGAAAACTTCCGGCCCATTAGTCTCATGAATATCGACGCTAAAATATTGAACAAGAT |
| | TCTCGCCAACAGAATCCAACAACATATTAAGAAATTGATACATCACGACCAGGTGGGGTTTATACCT |
| | GGCATGCAGGGCTGGTTTAACATCCGGAAGAGTATTAACGTCATTCAACACATTAATAGAGCTAAGG |
| | ATAAGAATCATATGATCATCTCTATAGACGCGGAAAAGGCATTCGATAAGATTCAGCAGCCATTTAT |
| | GCTCAAGACTCTGAACAAACTCGGCATCGACGGAACATATTTTAAGATTATTCGCGCAATTTACGAT |
| | AAGCCGACTGCTAACATTATCCTTAACGGCCAAAAGCTCGAGGCCTTTCCGCTCAAGACTGGAACCC |
| | GCCAAGGCTGTCCCCTCTCCCCGCTTTTGTTTAATATTGTACTCGAGGTGCTGGCTAGGGCTATTCG |
| | TCAAGAGAAAGAGATTAAAGGGATACAGCTCGGGAAGGAAGAGGTCAAGCTTTCCTTGTTCGCCGAT |
| | GATATGATTGTGTACCTGGAGAATCCTATTGTGTCTGCTCAGAACCTTCTTAAACTTATTTCTAACT |
| | TTAGCAAGGTCAGCGGCTATAAGATTAACGTCCAGAAATCTCAGGCCTTTCTGTACACAAATAATCG |
| | ACAGACCGAATCCCAGATAATGGGTGAGCTTCCGTTTGTCATAGCCAGCAAAAGGATAAAGTATCTC |
| | GGAATCCAGCTGACACGAGACGTTAAAGATTTGTTTAAGGAAAATTACAAGCCTCTCCTGAAAGAGA |
| | TTAAGGAAGATACTAATAAGTGGAAGAATATCCCCTGTTCATGGGTTGGCAGAATCAACATAGTGAA |
| | GATGGCAATACTTCCTAAAGTGATATATCGCTTTAACGCCATCCCAATTAAACTGCCTATGACCTTC |
| | TTTACGGAGCTCGAGAAAACAACCCTTAAATTTATATGGAATCAAAAGAGAGCAAGAATAGCGAAGT |
| | CCATCTTGAGCCAGAAGAATAAGGCCGGTGGGATTACTTTGCCTGATTTTAAGTTGTATTATAAAGC |
| | CACAGTAACTAAGACAGCCTGGTATTGGTATCAGAATAGAGACATCGACCAGTGGAATCGGACCGAA |
| | CCATCAGAGATAATGCCCCACATCTATAATTACCTTATATTCGATAAGCCAGAAAAGAATAAACAGT |
| | GGGGCAAAGACAGCCTCTTCAACAAGTGGTGTTGGGAGAATTGGCTGGCCATATGCCGGAAACTCAA |
| | GCTCGACCCCTTTCTTACACCCTACACTAAAATCAACAGTAGGTGGATCAAGGACTTGAATGTCAAG |
| | CCAAAGACTATAAAGACACTGGAAGAGAATCTTGGGATCACAATACAAGATATAGGCGTCGGCAAAG |
| | ATTTTATGTCAAAGACGCCCAAGGCCATGGCCACTAAGGATAAGATTGATAAGTGGGACCTTATTAA |
| | GCTCAAAAGCTTCTGTACTGCCAAGGAGACCACGATCAGAGTTAATAGGCAGCCCACTACATGGGAA |
| | AAGATTTTCGCCACTTATTCATCAGATAAGGGGTTGATAAGCAGAATATATAACGAGCTGAAGCAGA |
| | TCTACAAGAAGAAAACGAATAATCCCATCAAGAAGTGGGCAAAAGATATGAACAGGCATTTTAGCAA |
| | AGAGGATATCTACGCCGCGAAGAAGCATATGAAGAAGTGTAGTTCAAGCTTGGCCATTCGTGAGATG |
| | CAGATTAAGACGACCATGCGATACCACCTTACCCCAGTGAGGATGGCAATTATCAAGAAATCTGGCA |
| | ATAATAGATGTTGGCGGGGCTGTGGCGAGATTGGCACCCTGCTCCATTGCTGGTGGGATTGCAAGCT |
| | GGTGCAGCCGCTTTGGAAATCAGTCTGGCGCTTTCTGAGGGACCTCGAGCTTGAGATTCCCTTCGAT |
| | CCCGCAATTCCCTTGCTCGGAATCTATCCTAACGAATACAAGAGCTGTTGTTACAAGGATACGTGTA |
| | CCCGGATGTTCATCGCGGCCTTGTTTACGATAGCTAAGACGTGGAATCAGCCTAAGTGCCCCACAAT |
| | GATCGATTGGATCAAGAAAATGTGGCATATTTATACCATGGAGTATTACGCAGCAATTAAGAATGAC |
| | GAATTTATTTCCTTCGTTGGGACCTGGATGAAGCTGGAGACTATTATTCTGAGCAAGCTGTCTCAGG |
| | AGCAAAAGACAAAGCATAGAATCTTCTCTCTCATTGGTGGTAACGACTACAAAGACGATGACGACAA |
| | GTAAAGCGCTTCTAGAAGTTGTCTCCTCCTGCACTGACTGACTGATACAATCGATTTCTGGATCCGC |
| | AGGCCTAATCAACCTCTGGATTACAAAATTTGTGAAAGATTGACTGGTATTCTTAACTATGTTGCTC |
| | CTTTTACGCTATGTGGATACGCTGCTTTAATGCCTTTGTATCATGCTATTGCTTCCCGTATGGCTTT |
| | CATTTTCTCCTCCTTGTATAAATCCTGGTTGCTGTCTCTTTATGAGGAGTTGTGGCCCGTTGTCAGG |
| | CAACGTGGCGTGGTGTGCACTGTGTTTGCTGACGCAACCCCCACTGGTTGGGGCATTGCCACCACCT |
| | GTCAGCTCCTTTCCGGGACTTTCGCTTTCCCCCTCCCTATTGCCACGGCGGAACTCATCGCCGCCTG |
| | CCTTGCCCGCTGCTGGACAGGGGCTCGGCTGTTGGGCACTGACAATTCCGTGGTGTTGTCGGGGAAG |
| | CTGACGTCCTTTCCATGGCTGCTCGCCTGTGTTGCCACCTGGATTCTGCGCGGGACGTCCTTCTGCT |
| | ACGTCCCTTCGGCCCTCAATCCAGCGGACCTTCCTTCCCGCtgagagacacaaaaaattccaacaca |
| | ctattgcaatgaaaataaatttcctttattagccagaagtcagatgctcaaggggcttcatgatgtc |
| | cccataatttttggcagagggaaaaagatctcagtggtatttgtgagccagggcattggccttctga |
| | taggcagcctgcacctgaggagtgcggccgctttacttgtacagctcgtccatgccgagagtgatcc |
| | cggcggcggtcacgaactccagcaggaccatgtgatcgcgcttctcgttggggtctttgctcagggc |
| | ggactgggtgctcaggtagtggttgtcgggcagcagcacggggccgtcgccgatgggggtgttctgc |
| | tggtagtggtcggcgagctgcacgctgccgtcctcgatgttgtggcggatcttgaagttcaccttga |
| | tgccgttcttctgcttgtcggccatgatatagacgttgtggctgttgtagttgtactccagcttgtg |
| | ccccaggatgttgccgtcctccttgaagtcgatgcccttcagctcgatgcggttcaccagggtgtcg |
| | ccctcgaacttcacctcggcgcgggtcttgtagttgccgtcgtccttgaagaagatggtgcgctcct |
| | ggacgtagccttcgggcatggcggacttgaagaagtcgtgctgcttcatgtggtcggggtagcggct |
| | gaagcactgcacgccgtaggtcagggtggtcacgagggtgggccagggcacgggcagcttgccggtg |
| | gtgcagatgaacttcagggtcagcttgccgtaggtggcatcgccctcgccctcgccggacacgctga |
| | acttgtggccgtttacgtcgccgtccagctcgaccaggatgggcaccaccccggtgaacagctcctc |
| | gcccttgctcaccatggtggcgggatctgacggttcactaaaccagctctgcttatatagacctccc |
| | accgtacacgcctaccgcccatttgcgtcaatggggcggagttgttacgacattttggaaagtcccg |
| | ttgattttggtgccaaaacaaactcccattgacgtcaatggggtggagacttggaaatccccgtgag |
| | tcaaaccgctatccacgcccattgatgtactgccaaaaccgcatcaccatggtaatagcgatgacta |
| | atacgtagatgtactgccaagtaggaaagtcccataaggtcatgtactgggcataatgccaggcggg |
| | ccatttaccgtcattgacgtcaatagggggcgtacttggcatatgatacacttgatgtactgccaag |
| | tgggcagtttaccgtaaatactccacccattgacgtcaatggaaagtccctattggcgttactatgg |
| | gaacatacgtcattattgacgtcaatgggcgggggtcgttgggcggtcagccaggcgggccatttac |
| | cgtaagttatgtaacgGGCCTGCTGCCGGCTCTGCGGCCTCTTCCGCGTCTTCGCCTTCGCCCTCAG |
| | ACGAGTCGGATCTCCCTTTGGGCCGCCTCCCCGCCTGTCTAGCTTGACTGACTGAGATACAGCGTAC |
| | CTTCAGCTCACAGACATGATAAGATACATTGATGAGTTTGGACAAACCACAACTAGAATGCAGTGAA |
| | AAAAATGCTTTATTTGTGAAATTTGTGATGCTATTGCTTTATTTGTAACCATTATAAGCTGCAATAA |
| | ACAAGTTAACAACAACAATTGCATTCATTTTATGTTTCAGGTTCAGGGGGAGGTGTGGGAGGTTTTT |
| | TAAAGCAAGTAAAACCTCTACAAATGTGGTATTGGCCCATCTCTATCGGTATCGTAGCATAACCCCT |
| | TGGGGCCTCTAAACGGGTCTTGAGGGGTTTTTTGTGCCCCTCGGGCCGGATTGCTATCTACCGGCAT |
| | TGGCGCAGAAAAAAATGCCTGATGCGACGCTGCGCGTCTTATACTCCCACATATGCCAGATTCAGCA |
| | ACGGATACGGCTTCCCCAACTTGCCCACTTCCATACGTGTCCTCCTTACCAGAAATTTATCCTTAAG |
| | GTCGTCAGCTATCCTGCAGGCGATCTCTCGATTTCGATCAAGACATTCCTTTAATGGTCTTTTCTGG |
| | ACACCACTAGGGGTCAGAAGTAGTTCATCAAACTTTCTTCCCTCCCTAATCTCATTGGTTACCTTGG |
| | GCTATCGAAACTTAATTAAGCGATCTGCATCTCAATTAGTCAGCAACCATAGTCCCGCCCCTAACTC |
| | CGCCCATCCCGCCCCTAACTCCGCCCAGTTCCGCCCATTCTCCGCCCCATCGCTGACTAATTTTTTT |
| | TATTTATGCAGAGGCCGAGGCCGCCTCGGCCTCTGAGCTATTCCAGAAGTAGTGAGGAGGCTTTTTT |
| | GGAGGCCTAGGCTTTTGCAAAGGAGGTAGCCAACATGATTGAACAAGATGGATTGCACGCAGGTTCT |
| | CCCGCCGCTTGGGTGGAGAGGCTATTCGGCTATGACTGGGCACAACAGACAATCGGCTGCTCTGATG |
| | CCGCCGTGTTCCGGCTGTCAGCGCAGGGGCGCCCGGTTCTTTTTGTCAAGACCGACCTGTCCGGTGC |
| | CCTGAATGAACTCCAGGACGAGGCAGCGCGGCTATCGTGGCTGGCCACGACGGGCGTTCCTTGCGCA |
| | GCTGTGCTCGACGTTGTCACTGAAGCGGGAAGGGACTGGCTGCTATTGGGCGAAGTGCCGGGGCAGG |
| | ATCTCCTGTCATCTCACCTTGCTCCTGCCGAGAAAGTATCCATCATGGCTGATGCAATGCGGCGGCT |
| | GCATACGCTTGATCCGGCTACCTGCCCATTCGACCACCAAGCGAAACATCGCATCGAGCGAGCACGT |
| | ACTCGGATGGAAGCCGGTCTTGTCGATCAGGATGATCTGGACGAAGAGCATCAGGGGCTCGCGCCAG |
| | CCGAACTGTTCGCCAGGCTCAAGGCGCGGATGCCCGACGGCGAGGATCTCGTCGTGACCCACGGCGA |
| | TGCCTGCTTGCCGAATATCATGGTGGAAAATGGCCGCTTTTCTGGATTCATCGACTGTGGCCGGCTG |
| | GGTGTGGCGGACCGCTATCAGGACATAGCGTTGGCTACCCGTGATATTGCTGAAGAGCTTGGCGGCG |
| | AATGGGCTGACCGCTTCCTCGTGCTTTACGGTATCGCCGCTCCCGATTCGCAGCGCATCGCCTTCTA |
| | TCGCCTTCTTGACGAGTTCTTCTAGTATGTAAGCCCTGTGCCTTCTAGTTGCCAGCCATCTGTTGTT |
| | TGCCCCTCCCCCGTGCCTTCCTTGACCCTGGAAGGTGCCACTCCCACTGTCCTTTCCTAATAAAATG |
| | AGGAAATTGCATCGCATTGTCTGAGTAGGTGTCATTCTATTCTGGGGGGTGGGGTGGGGCAGGACAG |
| | CAAGGGGGAGGATTGGGAAGACAATAGCAGGCATGCTGGGGATGCGGTGGGCTCTATGGTTAATTAA |
| | CCAGTCAAGTCAGCTACTTGGCGAGATCGACTTGTCTGGGTTTCGACTACGCTCAGAATTGCGTCAG |
| | TCAAGTTCGATCTGGTCCTTGCTATTGCACCCGTTCTCCGATTACGAGTTTCATTTAAATCATGTGA |
| | GCAAAAGGCCAGCAAAAGGCCAGGAACCGTAAAAAGGCCGCGTTGCTGGCGTTTTTCCATAGGCTCC |
| | GCCCCCCTGACGAGCATCACAAAAATCGACGCTCAAGTCAGAGGTGGCGAAACCCGACAGGACTATA |
| | AAGATACCAGGCGTTTCCCCCTGGAAGCTCCCTCGTGCGCTCTCCTGTTCCGACCCTGCCGCTTACC |
| | GGATACCTGTCCGCCTTTCTCCCTTCGGGAAGCGTGGCGCTTTCTCATAGCTCACGCTGTAGGTATC |
| | TCAGTTCGGTGTAGGTCGTTCGCTCCAAGCTGGGCTGTGTGCACGAACCCCCCGTTCAGCCCGACCG |
| | CTGCGCCTTATCCGGTAACTATCGTCTTGAGTCCAACCCGGTAAGACACGACTTATCGCCACTGGCA |
| | GCAGCCACTGGTAACAGGATTAGCAGAGCGAGGTATGTAGGCGGTGCTACAGAGTTCTTGAAGTGGT |
| | GGCCTAACTACGGCTACACTAGAAGAACAGTATTTGGTATCTGCGCTCTGCTGAAGCCAGTTACCTT |
| | CGGAAAAAGAGTTGGTAGCTCTTGATCCGGCAAACAAACCACCGCTGGTAGCGGTGGTTTTTTTGTT |
| | TGCAAGCAGCAGATTACGCGCAGAAAAAAAGGATCTCAAGAAGATCCTTTGATCTTTTCTACGGGGT |
| | CTGACGCTCAGTGGAACGAAAACTCACGTTAAGGGATTTTGGTCATGAGATTATCAAAAAGGATCTT |
| | CACCTAGATCCTTTTAAATTAAAAATGAAGTTTTAAATCAATCTAAAGTATATATGAGTAAACTTGG |
| | TCTGACAGTTACCAATGCTTAATCAGTGAGGCACCTATCTCAGCGATCTGTCTATTTCGTTCATCCA |
| | TAGTTGCATTTAAATTTCCGAACTCTCCAAGGCCCTCGTC (SEQ ID NO: 98) |
| RET- | CAAACCTTTCGTCCGATCCATCTTGCAGGCTACCTCTCGAACGAACTATCGCAAGTCTCTTGGCCGG |
| 009 | CCTTGCGCCTTGGCTATTGCTTGGCAGCGCCTATCGCCAGGTATTACTCCAATCCCGAATATCCGAG |
| LINE1- | ATCGGGATCACCCGAGAGAAGTTCAACCTACATCCTCAATCCCGATCTATCCGAGATCCGAGGAATA |
| GFP | TCGAAATCGGGGCGCGCCTGGTGTACCGAGAACGATCCTCTCAGTGCGAGTCTCGACGATCCATATC |
| ORF1-N | GTTGCTTGGCAGTCAGCCAGTCGGAATCCAGCTTGGGACCCAGGAAGTCCAATCGTCAGATATTGTA |
| Nucleo- | CTCAAGCCTGGTCACGGCAGCGTACCGATCTGTTTAAACCTAGATATTGATAGTCTGATCGGTCAAC |
| plasmin | GTATAATCGAGTCCTAGCTTTTGCAAACATCTATCAAGAGACAGGATCAGCAGGAGGCTTTCGCATG |
| NLS | AGTATTCAACATTTCCGTGTCGCCCTTATTCCCTTTTTTGCGGCATTTTGCCTTCCTGTTTTTGCTC |
| Linker | ACCCAGAAACGCTGGTGAAAGTAAAAGATGCTGAAGATCAGTTGGGTGCGCGAGTGGGTTACATCGA |
| | ACTGGATCTCAACAGCGGTAAGATCCTTGAGAGTTTTCGCCCCGAAGAACGCTTTCCAATGATGAGC |
| | ACTTTTAAAGTTCTGCTATGTGGCGCGGTATTATCCCGTATTGACGCCGGGCAAGAGCAACTCGGTC |
| | GCCGCATACACTATTCTCAGAATGACTTGGTTGAGTATTCACCAGTCACAGAAAAGCATCTTACGGA |
| | TGGCATGACAGTAAGAGAATTATGCAGTGCTGCCATAACCATGAGTGATAACACTGCGGCCAACTTA |
| | CTTCTGACAACGATTGGAGGACCGAAGGAGCTAACCGCTTTTTTGCACAACATGGGGGATCATGTAA |
| | CTCGCCTTGATCGTTGGGAACCGGAGCTGAATGAAGCCATACCAAACGACGAGCGTGACACCACGAT |
| | GCCTGTAGCAATGGCAACAACCTTGCGTAAACTATTAACTGGCGAACTACTTACTCTAGCTTCCCGG |
| | CAACAGTTGATAGACTGGATGGAGGCGGATAAAGTTGCAGGACCACTTCTGCGCTCGGCCCTTCCGG |
| | CTGGCTGGTTTATTGCTGATAAATCTGGAGCCGGTGAGCGTGGGTCTCGCGGTATCATTGCAGCACT |
| | GGGGCCAGATGGTAAGCCCTCCCGTATCGTAGTTATCTACACGACGGGGAGTCAGGCAACTATGGAT |
| | GAACGAAATAGACAGATCGCTGAGATAGGTGCCTCACTGATTAAGCATTGGTAACCGATTCTAGGTG |
| | CATTGGCGCAGAAAAAAATGCCTGATGCGACGCTGCGCGTCTTATACTCCCACATATGCCAGATTCA |
| | GCAACGGATACGGCTTCCCCAACTTGCCCACTTCCATACGTGTCCTCCTTACCAGAAATTTATCCTT |
| | AAGATCGTTTAAACTCGACTCTGGCTCTATCGAATCTCCGTCGTTTCGAGCTTACGCGAACAGCCGT |
| | GGCGCTCATTTGCTCGTCGGGCATCGAATCTCGTCAGCTATCGTCAGCTTACCTTTTTGGCAGCGAT |
| | CGCGGCTCCCGACATCTTGGACCATTAGCTCCACAGGTATCTTCTTCCCTCTAGTGGTCATAACAGC |
| | AGCTTCAGCTACCTCTCAATTCAAAAAACCCCTCAAGACCCGTTTAGAGGCCCCAAGGGGTTATGCT |
| | ATCAATCGTTGCGTTACACACACAAAAAACCAACACACATCCATCTTCGATGGATAGCGATTTTATT |
| | ATCTAACTGCTGATCGAGTGTAGCCAGATCTAGTAATCAATTACGGGGTCATTAGTTCATAGCCCAT |
| | ATATGGAGTTCCGCGTTACATAACTTACGGTAAATGGCCCGCCTGGCTGACCGCCCAACGACCCCCG |
| | CCCATTGACGTCAATAATGACGTATGTTCCCATAGTAACGCCAATAGGGACTTTCCATTGACGTCAA |
| | TGGGTGGAGTATTTACGGTAAACTGCCCACTTGGCAGTACATCAAGTGTATCATATGCCAAGTACGC |
| | CCCCTATTGACGTCAATGACGGTAAATGGCCCGCCTGGCATTATGCCCAGTACATGACCTTATGGGA |
| | CTTTCCTACTTGGCAGTACATCTACGTATTAGTCATCGCTATTACCATGCTGATGCGGTTTTGGCAG |
| | TACATCAATGGGCGTGGATAGCGGTTTGACTCACGGGGATTTCCAAGTCTCCACCCCATTGACGTCA |
| | ATGGGAGTTTGTTTTGGCACCAAAATCAACGGGACTTTCCAAAATGTCGTAACAACTCCGCCCCATT |
| | GACGCAAATGGGCGGTAGGCGTGTACGGTGGGAGGTCTATATAAGCAGAGCTGGTTTAGTGAACCGT |
| | CAGATCAGATCTTTGTCGATCCTACCATCCACTCGACACACCCGCCAGCGGCCGCTAATACGACTCA |
| | CTATAGGGAGAAGTACTGCCACCATGaaaaggccggcggccacgaaaaaggccggccaggcaaaaaa |
| | gaaaaagggcggcggcagcGGCAAGAAGCAAAATCGCAAGACGGGGAATTCCAAGACACAATCCGCT |
| | AGCCCACCACCTAAAGAGCGTTCTAGCTCCCCTGCTACTGAGCAGTCCTGGATGGAAAACGACTTCG |
| | ATGAACTCCGGGAAGAGGGATTTAGGCGATCCAACTATTCAGAACTCCGCGAAGATATCCAGACAAA |
| | GGGGAAGGAAGTCGAGAATTTCGAGAAGAACCTCGAGGAGTGCATCACCCGTATCACAAACACTGAG |
| | AAATGTCTCAAAGAACTCATGGAACTTAAGACAAAAGCCAGGGAGCTTCGAGAGGAGTGTCGGAGTC |
| | TGAGATCCAGGTGTGACCAGCTCGAGGAGCGCGTGAGCGCGATGGAAGACGAGATGAACGAGATGAA |
| | AAGAGAGGGCAAATTCAGGGAGAAGCGCATTAAGAGGAACGAACAGAGTCTGCAGGAGATTTGGGAT |
| | TACGTCAAGAGGCCTAACCTGCGGTTGATCGGCGTCCCCGAGAGCGACGTAGAAAACGGGACTAAAC |
| | TGGAGAATACACTTCAAGACATCATTCAAGAAAATTTTCCAAACCTGGCTCGGCAAGCTAATGTGCA |
| | AATCCAAGAGATCCAACGCACACCCCAGCGGTATAGCTCTCGGCGTGCCACCCCTAGGCATATTATC |
| | GTGCGCTTTACTAAGGTGGAGATGAAAGAGAAGATGCTGCGAGCCGCTCGGGAAAAGGGAAGGGTGA |
| | CTTTGAAGGGCAAACCTATTCGGCTGACGGTTGACCTTAGCGCCGAGACACTCCAGGCACGCCGGGA |
| | ATGGGGCCCCATCTTTAATATCCTGAAGGAGAAGAACTTCCAGCCACGAATCTCTTACCCTGCAAAG |
| | TTGAGTTTTATCTCCGAGGGTGAGATTAAGTATTTCATCGATAAACAGATGCTGCGAGACTTCGTGA |
| | CAACTCGCCCAGCTCTCAAGGAACTGCTCAAAGAGGCTCTTAATATGGAGCGCAATAATAGATATCA |
| | ACCCTTGCAGAACCACGCAAAGATGTGAGACAGCCGTCAGACCATCAAGACTAGGAAGAAACTGCAT |
| | CAACTAATGAGCAAAATCACCAGCTAACATCATAGTATACATGACCGGCTCTAACTCACATATCACC |
| | ATCCTTACACTTAACATTAACGGCCTCAACTCAGCTATCAAGCGCCATCGGCTGGCCAGCTGGATCA |
| | AATCACAGGATCCAAGCGTTTGTTGCATCCAAGAGACCCACCTGACCTGTAGAGATACTCACCGCCT |
| | CAAGATCAAGGGATGGCGAAAGATTTATCAGGCGAACGGTAAGCAGAAGAAAGCCGGAGTCGCAATT |
| | CTGGTCTCAGACAAGACGGATTTCAAGCCCACCAAAATTAAGCGTGATAAGGAAGGTCACTATATTA |
| | TGGTGAAAGGCAGCATACAGCAGGAAGAACTTACCATATTGAACATCTACGCGCCAAACACCGGCGC |
| | ACCTCGCTTTATCAAACAGGTCCTGTCCGATCTGCAGCGAGATCTGGATTCTCATACGTTGATTATG |
| | GGTGATTTCAATACACCATTGAGCACCCTGGATCGCAGCACCAGGCAAAAGGTAAATAAAGACACGC |
| | AAGAGCTCAATAGCGCACTGCATCAGGCAGATCTCATTGATATTTATCGCACTCTTCATCCTAAGAG |
| | TACCGAGTACACATTCTTCAGCGCCCCACATCATACATACTCAAAGATCGATCATATCGTCGGCTCA |
| | AAGGCTCTGCTGTCAAAGTGCAAGCGCACAGAGATAATTACAAATTACCTGTCAGATCATAGCGCGA |
| | TCAAGCTCGAGCTGAGAATCAAGAACCTGACCCAGAGCCGGAGTACCACTTGGAAGCTTAATAACCT |
| | GCTGCTCAACGATTATTGGGTCCACAATGAGATGAAGGCAGAGATTAAAATGTTCTTCGAAACAAAT |
| | GAGAATAAGGATACTACCTATCAAAACCTTTGGGATGCCTTTAAGGCCGTCTGCAGAGGCAAGTTCA |
| | TCGCCCTCAACGCCTATAAAAGAAAACAAGAGAGATCTAAGATCGATACTCTCACCTCTCAGCTGAA |
| | GGAGTTGGAGAAACAGGAACAGACCCACTCCAAGGCGTCAAGACGGCAGGAGATCACAAAGATTCGC |
| | GCCGAGTTGAAAGAGATCGAAACCCAAAAGACTCTTCAGAAAATTAACGAGTCTCGTAGTTGGTTCT |
| | TCGAGCGGATTAATAAGATAGACAGACCTCTGGCACGACTGATTAAGAAGAAGCGCGAAAAGAACCA |
| | GATTGATACCATCAAGAACGACAAGGGCGACATCACTACTGACCCGACCGAGATCCAGACCACTATT |
| | CGGGAGTATTATAAGCATTTGTATGCTAACAAGCTTGAGAACCTGGAAGAGATGGACACTTTTCTGG |
| | ATACCTATACTCTGCCACGGCTTAATCAAGAGGAAGTCGAGTCCCTCAACCGCCCAATTACAGGAAG |
| | CGAGATTGTGGCCATAATTAACTCCCTGCCGACAAAGAAATCTCCTGGTCCGGACGGGTTTACAGCT |
| | GAGTTTTATCAACGGTATATGGAAGAGCTTGTACCGTTTCTGCTCAAGCTCTTTCAGTCTATAGAAA |
| | AGGAAGGCATCTTGCCCAATTCCTTCTACGAAGCTTCTATAATACTTATTCCCAAACCAGGACGCGA |
| | TACCACAAAGAAGGAAAACTTCCGGCCCATTAGTCTCATGAATATCGACGCTAAAATATTGAACAAG |
| | ATTCTCGCCAACAGAATCCAACAACATATTAAGAAATTGATACATCACGACCAGGTGGGGTTTATAC |
| | CTGGCATGCAGGGCTGGTTTAACATCCGGAAGAGTATTAACGTCATTCAACACATTAATAGAGCTAA |
| | GGATAAGAATCATATGATCATCTCTATAGACGCGGAAAAGGCATTCGATAAGATTCAGCAGCCATTT |
| | ATGCTCAAGACTCTGAACAAACTCGGCATCGACGGAACATATTTTAAGATTATTCGCGCAATTTACG |
| | ATAAGCCGACTGCTAACATTATCCTTAACGGCCAAAAGCTCGAGGCCTTTCCGCTCAAGACTGGAAC |
| | CCGCCAAGGCTGTCCCCTCTCCCCGCTTTTGTTTAATATTGTACTCGAGGTGCTGGCTAGGGCTATT |
| | CGTCAAGAGAAAGAGATTAAAGGGATACAGCTCGGGAAGGAAGAGGTCAAGCTTTCCTTGTTCGCCG |
| | ATGATATGATTGTGTACCTGGAGAATCCTATTGTGTCTGCTCAGAACCTTCTTAAACTTATTTCTAA |
| | CTTTAGCAAGGTCAGCGGCTATAAGATTAACGTCCAGAAATCTCAGGCCTTTCTGTACACAAATAAT |
| | CGACAGACCGAATCCCAGATAATGGGTGAGCTTCCGTTTGTCATAGCCAGCAAAAGGATAAAGTATC |
| | TCGGAATCCAGCTGACACGAGACGTTAAAGATTTGTTTAAGGAAAATTACAAGCCTCTCCTGAAAGA |
| | GATTAAGGAAGATACTAATAAGTGGAAGAATATCCCCTGTTCATGGGTTGGCAGAATCAACATAGTG |
| | AAGATGGCAATACTTCCTAAAGTGATATATCGCTTTAACGCCATCCCAATTAAACTGCCTATGACCT |
| | TCTTTACGGAGCTCGAGAAAACAACCCTTAAATTTATATGGAATCAAAAGAGAGCAAGAATAGCGAA |
| | GTCCATCTTGAGCCAGAAGAATAAGGCCGGTGGGATTACTTTGCCTGATTTTAAGTTGTATTATAAA |
| | GCCACAGTAACTAAGACAGCCTGGTATTGGTATCAGAATAGAGACATCGACCAGTGGAATCGGACCG |
| | AACCATCAGAGATAATGCCCCACATCTATAATTAGCTTATATTCGATAAGCCAGAAAAGAATAAACA |
| | GTGGGGCAAAGACAGCCTCTTCAACAAGTGGTGTTGGGAGAATTGGCTGGCCATATGCCGGAAACTC |
| | AAGCTCGACCCCTTTCTTACACCCTACACTAAAATCAACAGTAGGTGGATCAAGGACTTGAATGTCA |
| | AGCCAAAGACTATAAAGACACTGGAAGAGAATCTTGGGATCACAATACAAGATATAGGCGTCGGCAA |
| | AGATTTTATGTCAAAGACGCCCAAGGCCATGGCCACTAAGGATAAGATTGATAAGTGGGACCTTATT |
| | AAGCTCAAAAGCTTCTGTACTGCCAAGGAGACCACGATCAGAGTTAATAGGCAGCCCACTACATGGG |
| | AAAAGATTTTCGCCACTTATTCATCAGATAAGGGGTTGATAAGCAGAATATATAACGAGCTGAAGCA |
| | GATCTACAAGAAGAAAACGAATAATCCCATCAAGAAGTGGGCAAAAGATATGAACAGGCATTTTAGC |
| | AAAGAGGATATCTACGCCGCGAAGAAGCATATGAAGAAGTGTAGTTCAAGCTTGGCCATTCGTGAGA |
| | TGCAGATTAAGACGACCATGCGATACCACCTTACCCCAGTGAGGATGGCAATTATCAAGAAATCTGG |
| | CAATAATAGATGTTGGCGGGGCTGTGGCGAGATTGGCACCCTGCTCCATTGCTGGTGGGATTGCAAG |
| | CTGGTGCAGCCGCTTTGGAAATCAGTCTGGCGCTTTCTGAGGGACCTCGAGCTTGAGATTCCCTTCG |
| | ATCCCGCAATTCCCTTGCTCGGAATCTATCCTAACGAATACAAGAGCTGTTGTTACAAGGATACGTG |
| | TACCCGGATGTTCATCGCGGCCTTGTTTACGATAGCTAAGACGTGGAATCAGCCTAAGTGCCCCACA |
| | ATGATCGATTGGATCAAGAAAATGTGGCATATTTATACCATGGAGTATTACGCAGCAATTAAGAATG |
| | ACGAATTTATTTCCTTCGTTGGGACCTGGATGAAGCTGGAGACTATTATTCTGAGCAAGCTGTCTCA |
| | GGAGCAAAAGACAAAGCATAGAATCTTCTCTCTCATTGGTGGTAACGACTACAAAGACGATGACGAC |
| | AAGTAAAGCGCTTCTAGAAGTTGTCTCCTCCTGCACTGACTGACTGATACAATCGATTTCTGGATCC |
| | GCAGGCCTAATCAACCTCTGGATTACAAAATTTGTGAAAGATTGACTGGTATTCTTAACTATGTTGC |
| | TCCTTTTACGCTATGTGGATACGCTGCTTTAATGCCTTTGTATCATGCTATTGCTTCCCGTATGGCT |
| | TTCATTTTCTCCTCCTTGTATAAATCCTGGTTGCTGTCTCTTTATGAGGAGTTGTGGCCCGTTGTCA |
| | GGCAACGTGGCGTGGTGTGCACTGTGTTTGCTGACGCAACCCCCACTGGTTGGGGCATTGCCACCAC |
| | CTGTCAGCTCCTTTCCGGGACTTTCGCTTTCCCCCTCCCTATTGCCACGGCGGAACTCATCGCCGCC |
| | TGCCTTGCCCGCTGCTGGACAGGGGCTCGGCTGTTGGGCACTGACAATTCCGTGGTGTTGTCGGGGA |
| | AGCTGACGTCCTTTCCATGGCTGCTCGCCTGTGTTGCCACCTGGATTCTGCGCGGGACGTCCTTCTG |
| | CTACGTCCCTTCGGCCCTCAATCCAGCGGACCTTCCTTCCCGCtgagagacacaaaaaattccaaca |
| | cactattgcaatgaaaataaatttcctttattagccagaagtcagatgctcaaggggcttcatgatg |
| | tccccataatttttggcagagggaaaaagatctcagtggtatttgtgagccagggcattggccttct |
| | gataggcagcctgcacctgaggagtgcggccgctttacttgtacagctcgtccatgccgagagtgat |
| | cccggcggcggtcacgaactccagcaggaccatgtgatcgcgcttctcgttggggtctttgctcagg |
| | gcggactgggtgctcaggtagtggttgtcgggcagcagcacggggccgtcgccgatgggggtgttct |
| | gctggtagtggtcggcgagctgcacgctgccgtcctcgatgttgtggcggatcttgaagttcacctt |
| | gatgccgttcttctgcttgtcggccatgatatagacgttgtggctgttgtagttgtactccagcttg |
| | tgccccaggatgttgccgtcctccttgaagtcgatgcccttcagctcgatgcggttcaccagggtgt |
| | cgccctcgaacttcacctcggcgcgggtcttgtagttgccgtcgtccttgaagaagatggtgcgctc |
| | ctggacgtagccttcgggcatggcggacttgaagaagtcgtgctgcttcatgtggtcggggtagcgg |
| | ctgaagcactgcacgccgtaggtcagggtggtcacgagggtgggccagggcacgggcagcttgccgg |
| | tggtgcagatgaacttcagggtcagcttgccgtaggtggcatcgccctcgccctcgccggacacgct |
| | gaacttgtggccgtttacgtcgccgtccagctcgaccaggatgggcaccaccccggtgaacagctcc |
| | tcgcccttgctcaccatggtggcgggatctgacggttcactaaaccagctctgcttatatagacctc |
| | ccaccgtacacgcctaccgcccatttgcgtcaatggggcggagttgttacgacattttggaaagtcc |
| | cgttgattttggtgccaaaacaaactcccattgacgtcaatggggtggagacttggaaatccccgtg |
| | agtcaaaccgctatccacgcccattgatgtactgccaaaaccgcatcaccatggtaatagcgatgac |
| | taatacgtagatgtactgccaagtaggaaagtcccataaggtcatgtactgggcataatgccaggcg |
| | ggccatttaccgtcattgacgtcaatagggggcgtacttggcatatgatacacttgatgtactgcca |
| | agtgggcagtttaccgtaaatactccacccattgacgtcaatggaaagtccctattggcgttactat |
| | gggaacatacgtcattattgacgtcaatgggcgggggtcgttgggcggtcagccaggcgggccattt |
| | accgtaagttatgtaacgGGCCTGCTGCCGGCTCTGCGGCCTCTTCCGCGTCTTCGCCTTCGCCCTC |
| | AGACGAGTCGGATCTCCCTTTGGGCCGCCTCCCCGCCTGTCTAGCTTGACTGACTGAGATACAGCGT |
| | ACCTTCAGCTCACAGACATGATAAGATACATTGATGAGTTTGGACAAACCACAACTAGAATGCAGTG |
| | AAAAAAATGCTTTATTTGTGAAATTTGTGATGCTATTGCTTTATTTGTAACCATTATAAGCTGCAAT |
| | AAACAAGTTAACAACAACAATTGCATTCATTTTATGTTTCAGGTTCAGGGGGAGGTGTGGGAGGTTT |
| | TTTAAAGCAAGTAAAACCTCTACAAATGTGGTATTGGCCCATCTCTATCGGTATCGTAGCATAACCC |
| | CTTGGGGCCTCTAAACGGGTCTTGAGGGGTTTTTTGTGCCCCTCGGGCCGGATTGCTATCTACCGGC |
| | ATTGGCGCAGAAAAAAATGCCTGATGCGACGCTGCGCGTCTTATACTCCCACATATGCCAGATTCAG |
| | CAACGGATACGGCTTCCCCAACTTGCCCACTTCCATACGTGTCCTCCTTACCAGAAATTTATCCTTA |
| | AGGTCGTCAGCTATCCTGCAGGCGATCTCTCGATTTCGATCAAGACATTCCTTTAATGGTCTTTTCT |
| | GGACACCACTAGGGGTCAGAAGTAGTTCATCAAACTTTCTTCCCTCCCTAATCTCATTGGTTACCTT |
| | GGGCTATCGAAACTTAATTAAGCGATCTGCATCTCAATTAGTCAGCAACCATAGTCCCGCCCCTAAC |
| | TCCGCCCATCCCGCCCCTAACTCCGCCCAGTTCCGCCCATTCTCCGCCCCATCGCTGACTAATTTTT |
| | TTTATTTATGCAGAGGCCGAGGCCGCCTCGGCCTCTGAGCTATTCCAGAAGTAGTGAGGAGGCTTTT |
| | TTGGAGGCCTAGGCTTTTGCAAAGGAGGTAGCCAACATGATTGAACAAGATGGATTGCACGCAGGTT |
| | CTCCCGCCGCTTGGGTGGAGAGGCTATTCGGCTATGACTGGGCACAACAGACAATCGGCTGCTCTGA |
| | TGCCGCCGTGTTCCGGCTGTCAGCGCAGGGGCGCCCGGTTCTTTTTGTCAAGACCGACCTGTCCGGT |
| | GCCCTGAATGAACTCCAGGACGAGGCAGCGCGGCTATCGTGGCTGGCCACGACGGGCGTTCCTTGCG |
| | CAGCTGTGCTCGACGTTGTCACTGAAGCGGGAAGGGACTGGCTGCTATTGGGCGAAGTGCCGGGGCA |
| | GGATCTCCTGTCATCTCACCTTGCTCCTGCCGAGAAAGTATCCATCATGGCTGATGCAATGCGGCGG |
| | CTGCATACGCTTGATCCGGCTACCTGCCCATTCGACCACCAAGCGAAACATCGCATCGAGCGAGCAC |
| | GTACTCGGATGGAAGCCGGTCTTGTCGATCAGGATGATCTGGACGAAGAGCATCAGGGGCTCGCGCC |
| | AGCCGAACTGTTCGCCAGGCTCAAGGCGCGGATGCCCGACGGCGAGGATCTCGTCGTGACCCACGGC |
| | GATGCCTGCTTGCCGAATATCATGGTGGAAAATGGCCGCTTTTCTGGATTCATCGACTGTGGCCGGC |
| | TGGGTGTGGCGGACCGCTATCAGGACATAGCGTTGGCTACCCGTGATATTGCTGAAGAGCTTGGCGG |
| | CGAATGGGCTGACCGCTTCCTCGTGCTTTACGGTATCGCCGCTCCCGATTCGCAGCGCATCGCCTTC |
| | TATCGCCTTCTTGACGAGTTCTTCTAGTATGTAAGCCCTGTGCCTTCTAGTTGCCAGCCATCTGTTG |
| | TTTGCCCCTCCCCCGTGCCTTCCTTGACCCTGGAAGGTGCCACTCCCACTGTCCTTTCCTAATAAAA |
| | TGAGGAAATTGCATCGCATTGTCTGAGTAGGTGTCATTCTATTCTGGGGGGTGGGGTGGGGCAGGAC |
| | AGCAAGGGGGAGGATTGGGAAGACAATAGCAGGCATGCTGGGGATGCGGTGGGCTCTATGGTTAATT |
| | AACCAGTCAAGTCAGCTACTTGGCGAGATCGACTTGTCTGGGTTTCGACTACGCTCAGAATTGCGTC |
| | AGTCAAGTTCGATCTGGTCCTTGCTATTGCACCCGTTCTCCGATTACGAGTTTCATTTAAATCATGT |
| | GAGCAAAAGGCCAGCAAAAGGCCAGGAACCGTAAAAAGGCCGCGTTGCTGGCGTTTTTCCATAGGCT |
| | CCGCCCCCCTGACGAGCATCACAAAAATCGACGCTCAAGTCAGAGGTGGCGAAACCCGACAGGACTA |
| | TAAAGATACCAGGCGTTTCCCCCTGGAAGCTCCCTCGTGCGCTCTCCTGTTCCGACCCTGCCGCTTA |
| | CCGGATACCTGTCCGCCTTTCTCCCTTCGGGAAGCGTGGCGCTTTCTCATAGCTCACGCTGTAGGTA |
| | TCTCAGTTCGGTGTAGGTCGTTCGCTCCAAGCTGGGCTGTGTGCACGAACCCCCCGTTCAGCCCGAC |
| | CGCTGCGCCTTATCCGGTAACTATCGTCTTGAGTCCAACCCGGTAAGACACGACTTATCGCCACTGG |
| | CAGCAGCCACTGGTAACAGGATTAGCAGAGCGAGGTATGTAGGCGGTGCTACAGAGTTCTTGAAGTG |
| | GTGGCCTAACTACGGCTACACTAGAAGAACAGTATTTGGTATCTGCGCTCTGCTGAAGCCAGTTACC |
| | TTCGGAAAAAGAGTTGGTAGCTCTTGATCCGGCAAACAAACCACCGCTGGTAGCGGTGGTTTTTTTG |
| | TTTGCAAGCAGCAGATTACGCGCAGAAAAAAAGGATCTCAAGAAGATCCTTTGATCTTTTCTACGGG |
| | GTCTGACGCTCAGTGGAACGAAAACTCACGTTAAGGGATTTTGGTCATGAGATTATCAAAAAGGATC |
| | TTCACCTAGATCCTTTTAAATTAAAAATGAAGTTTTAAATCAATCTAAAGTATATATGAGTAAACTT |
| | GGTCTGACAGTTACCAATGCTTAATCAGTGAGGCACCTATCTCAGCGATCTGTCTATTTCGTTCATC |
| | CATAGTTGCATTTAAATTTCCGAACTCTCCAAGGCCCTCGTCGGAAAATCTT (SEQ ID NO: |
| | 99) |
| RET- | GGGGCGCGCCTGGTGTACCGAGAACGATCCTCTCAGTGCGAGTCTCGACGATCCATATCGTTGCTTG |
| 010 | GCAGTCAGCCAGTCGGAATCCAGCTTGGGACCCAGGAAGTCCAATCGTCAGATATTGTACTCAAGCC |
| LINE1- | TGGTCACGGCAGCGTACCGATCTGTTTAAACCTAGATATTGATAGTCTGATCGGTCAACGTATAATC |
| GFP | GAGTCCTAGCTTTTGCAAACATCTATCAAGAGACAGGATCAGCAGGAGGCTTTCGCATGAGTATTCA |
| ORF2- | ACATTTCCGTGTCGCCCTTATTCCCTTTTTTGCGGCATTTTGCCTTCCTGTTTTTGCTCACCCAGAA |
| NSV40- | ACGCTGGTGAAAGTAAAAGATGCTGAAGATCAGTTGGGTGCGCGAGTGGGTTACATCGAACTGGATC |
| NLS | TCAACAGCGGTAAGATCCTTGAGAGTTTTCGCCCCGAAGAACGCTTTCCAATGATGAGCACTTTTAA |
| | AGTTCTGCTATGTGGCGCGGTATTATCCCGTATTGACGCCGGGCAAGAGCAACTCGGTCGCCGCATA |
| | CACTATTCTCAGAATGACTTGGTTGAGTATTCACCAGTCACAGAAAAGCATCTTACGGATGGCATGA |
| | CAGTAAGAGAATTATGCAGTGCTGCCATAACCATGAGTGATAACACTGCGGCCAACTTAGTTCTGAC |
| | AACGATTGGAGGACCGAAGGAGCTAACCGCTTTTTTGCACAACATGGGGGATCATGTAACTCGCCTT |
| | GATCGTTGGGAACCGGAGCTGAATGAAGCCATACCAAACGACGAGCGTGACACCACGATGCCTGTAG |
| | CAATGGCAACAACCTTGCGTAAACTATTAACTGGCGAACTACTTACTCTAGCTTCCCGGCAACAGTT |
| | GATAGACTGGATGGAGGCGGATAAAGTTGCAGGACCACTTCTGCGCTCGGCCCTTCCGGCTGGCTGG |
| | TTTATTGCTGATAAATCTGGAGCCGGTGAGCGTGGGTCTCGCGGTATCATTGCAGCACTGGGGCCAG |
| | ATGGTAAGCCCTCCCGTATCGTAGTTATCTACACGACGGGGAGTCAGGCAACTATGGATGAACGAAA |
| | TAGACAGATCGCTGAGATAGGTGCCTCACTGATTAAGCATTGGTAACCGATTCTAGGTGCATTGGCG |
| | CAGAAAAAAATGCCTGATGCGACGCTGCGCGTCTTATACTCCCACATATGCCAGATTCAGCAACGGA |
| | TACGGCTTCCCCAACTTGCCCACTTCCATACGTGTCCTCCTTACCAGAAATTTATCCTTAAGATCGT |
| | TTAAACTCGACTCTGGCTCTATCGAATCTCCGTCGTTTCGAGCTTACGCGAACAGCCGTGGCGCTCA |
| | TTTGCTCGTCGGGCATCGAATCTCGTCAGCTATCGTCAGCTTACCTTTTTGGCAGCGATCGCGGCTC |
| | CCGACATCTTGGACCATTAGCTCCACAGGTATCTTCTTCCCTCTAGTGGTCATAACAGCAGCTTCAG |
| | CTACCTCTCAATTCAAAAAACCCCTCAAGACCCGTTTAGAGGCCCCAAGGGGTTATGCTATCAATCG |
| | TTGCGTTACACACACAAAAAACCAACACACATCCATCTTCGATGGATAGCGATTTTATTATCTAACT |
| | GCTGATCGAGTGTAGCCAGATCTAGTAATCAATTACGGGGTCATTAGTTCATAGCCCATATATGGAG |
| | TTCCGCGTTACATAACTTACGGTAAATGGCCCGCCTGGCTGACCGCCCAACGACCCCCGCCCATTGA |
| | CGTCAATAATGACGTATGTTCCCATAGTAACGCCAATAGGGACTTTCCATTGACGTCAATGGGTGGA |
| | GTATTTACGGTAAACTGCCCACTTGGCAGTACATCAAGTGTATCATATGCCAAGTACGCCCCCTATT |
| | GACGTCAATGACGGTAAATGGCCCGCCTGGCATTATGCCCAGTACATGACCTTATGGGACTTTCCTA |
| | CTTGGCAGTACATCTACGTATTAGTCATCGCTATTACCATGCTGATGCGGTTTTGGCAGTACATCAA |
| | TGGGCGTGGATAGCGGTTTGACTCACGGGGATTTCCAAGTCTCCACCCCATTGACGTCAATGGGAGT |
| | TTGTTTTGGCACCAAAATCAACGGGACTTTCCAAAATGTCGTAACAACTCCGCCCCATTGACGCAAA |
| | TGGGCGGTAGGCGTGTACGGTGGGAGGTCTATATAAGCAGAGCTGGTTTAGTGAACCGTCAGATCAG |
| | ATCTTTGTCGATCCTACCATCCACTCGACACACCCGCCAGCGGCCGCTAATACGACTCACTATAGGG |
| | AGAAGTACTGCCACCATGGGCAAGAAGCAAAATCGCAAGACGGGGAATTCCAAGACACAATCCGCTA |
| | GCCCACCACCTAAAGAGCGTTCTAGCTCCCCTGCTACTGAGCAGTCCTGGATGGAAAACGACTTCGA |
| | TGAACTCCGGGAAGAGGGATTTAGGCGATCCAACTATTCAGAACTCCGCGAAGATATCCAGACAAAG |
| | GGGAAGGAAGTCGAGAATTTCGAGAAGAACCTCGAGGAGTGCATCACCCGTATCACAAACACTGAGA |
| | AATGTCTCAAAGAACTCATGGAACTTAAGACAAAAGCCAGGGAGCTTCGAGAGGAGTGTCGGAGTCT |
| | GAGATCCAGGTGTGACCAGCTCGAGGAGCGCGTGAGCGCGATGGAAGACGAGATGAACGAGATGAAA |
| | AGAGAGGGCAAATTCAGGGAGAAGCGCATTAAGAGGAACGAACAGAGTCTGCAGGAGATTTGGGATT |
| | ACGTCAAGAGGCCTAACCTGCGGTTGATCGGCGTCCCCGAGAGCGACGTAGAAAACGGGACTAAACT |
| | GGAGAATACACTTCAAGACATCATTCAAGAAAATTTTCCAAACCTGGCTCGGCAAGCTAATGTGCAA |
| | ATCCAAGAGATCCAACGCACACCCCAGCGGTATAGCTCTCGGCGTGCCACCCCTAGGCATATTATCG |
| | TGCGCTTTACTAAGGTGGAGATGAAAGAGAAGATGCTGCGAGCCGCTCGGGAAAAGGGAAGGGTGAC |
| | TTTGAAGGGCAAACCTATTCGGCTGACGGTTGACCTTAGCGCCGAGACACTCCAGGCACGCCGGGAA |
| | TGGGGCCCCATCTTTAATATCCTGAAGGAGAAGAACTTCCAGCCACGAATCTCTTACCCTGCAAAGT |
| | TGAGTTTTATCTCCGAGGGTGAGATTAAGTATTTCATCGATAAACAGATGCTGCGAGACTTCGTGAC |
| | AACTCGCCCAGCTCTCAAGGAACTGCTCAAAGAGGCTCTTAATATGGAGCGCAATAATAGATATCAA |
| | CCCTTGCAGAACCACGCAAAGATGTGAGACAGCCGTCAGACCATCAAGACTAGGAAGAAACTGCATC |
| | AACTAATGAGCAAAATCACCAGCTAACATCATAGTATACATGccaaagaagaagcggaaggtcACCG |
| | GCTCTAACTCACATATCACCATCCTTACACTTAACATTAACGGCCTCAACTCAGCTATCAAGCGCCA |
| | TCGGCTGGCCAGCTGGATCAAATCACAGGATCCAAGCGTTTGTTGCATCCAAGAGACCCACCTGACC |
| | TGTAGAGATACTCACCGCCTCAAGATCAAGGGATGGCGAAAGATTTATCAGGCGAACGGTAAGCAGA |
| | AGAAAGCCGGAGTCGCAATTCTGGTCTCAGACAAGACGGATTTCAAGCCCACCAAAATTAAGCGTGA |
| | TAAGGAAGGTCACTATATTATGGTGAAAGGCAGCATACAGCAGGAAGAACTTACCATATTGAACATC |
| | TACGCGCCAAACACCGGCGCACCTCGCTTTATCAAACAGGTCCTGTCCGATCTGCAGCGAGATCTGG |
| | ATTCTCATACGTTGATTATGGGTGATTTCAATACACCATTGAGCACCCTGGATCGCAGCACCAGGCA |
| | AAAGGTAAATAAAGACACGCAAGAGCTCAATAGCGCACTGCATCAGGCAGATCTCATTGATATTTAT |
| | CGCACTCTTCATCCTAAGAGTACCGAGTACACATTCTTCAGCGCCCCACATCATACATACTCAAAGA |
| | TCGATCATATCGTCGGCTCAAAGGCTCTGCTGTCAAAGTGCAAGCGCACAGAGATAATTACAAATTA |
| | CCTGTCAGATCATAGCGCGATCAAGCTCGAGCTGAGAATCAAGAACCTGACCCAGAGCCGGAGTACC |
| | ACTTGGAAGCTTAATAACCTGCTGCTCAACGATTATTGGGTCCACAATGAGATGAAGGCAGAGATTA |
| | AAATGTTCTTCGAAACAAATGAGAATAAGGATACTACCTATCAAAACCTTTGGGATGCCTTTAAGGC |
| | CGTCTGCAGAGGCAAGTTCATCGCCCTCAACGCCTATAAAAGAAAACAAGAGAGATCTAAGATCGAT |
| | ACTCTCACCTCTCAGCTGAAGGAGTTGGAGAAACAGGAACAGACCCACTCCAAGGCGTCAAGACGGC |
| | AGGAGATCACAAAGATTCGCGCCGAGTTGAAAGAGATCGAAACCCAAAAGACTCTTCAGAAAATTAA |
| | CGAGTCTCGTAGTTGGTTCTTCGAGCGGATTAATAAGATAGACAGACCTCTGGCACGACTGATTAAG |
| | AAGAAGCGCGAAAAGAACCAGATTGATACCATCAAGAACGACAAGGGCGACATCACTACTGACCCGA |
| | CCGAGATCCAGACCACTATTCGGGAGTATTATAAGCATTTGTATGCTAACAAGCTTGAGAACCTGGA |
| | AGAGATGGACACTTTTCTGGATACCTATACTCTGCCACGGCTTAATCAAGAGGAAGTCGAGTCCCTC |
| | AACCGCCCAATTACAGGAAGCGAGATTGTGGCCATAATTAACTCCCTGCCGACAAAGAAATCTCCTG |
| | GTCCGGACGGGTTTACAGCTGAGTTTTATCAACGGTATATGGAAGAGCTTGTACCGTTTCTGCTCAA |
| | GCTCTTTCAGTCTATAGAAAAGGAAGGCATCTTGCCCAATTCCTTCTACGAAGCTTCTATAATACTT |
| | ATTCCCAAACCAGGACGCGATACCACAAAGAAGGAAAACTTCCGGCCCATTAGTCTCATGAATATCG |
| | ACGCTAAAATATTGAACAAGATTCTCGCCAACAGAATCCAACAACATATTAAGAAATTGATACATCA |
| | CGACCAGGTGGGGTTTATACCTGGCATGCAGGGCTGGTTTAACATCCGGAAGAGTATTAACGTCATT |
| | CAACACATTAATAGAGCTAAGGATAAGAATCATATGATCATCTCTATAGACGCGGAAAAGGCATTCG |
| | ATAAGATTCAGCAGCCATTTATGCTCAAGACTCTGAACAAACTCGGCATCGACGGAACATATTTTAA |
| | GATTATTCGCGCAATTTACGATAAGCCGACTGCTAACATTATCCTTAACGGCCAAAAGCTCGAGGCC |
| | TTTCCGCTCAAGACTGGAACCCGCCAAGGCTGTCCCCTCTCCCCGCTTTTGTTTAATATTGTACTCG |
| | AGGTGCTGGCTAGGGCTATTCGTCAAGAGAAAGAGATTAAAGGGATACAGCTCGGGAAGGAAGAGGT |
| | CAAGCTTTCCTTGTTCGCCGATGATATGATTGTGTACCTGGAGAATCCTATTGTGTCTGCTCAGAAC |
| | CTTCTTAAACTTATTTCTAACTTTAGCAAGGTCAGCGGCTATAAGATTAACGTCCAGAAATCTCAGG |
| | CCTTTCTGTACACAAATAATCGACAGACCGAATCCCAGATAATGGGTGAGCTTCCGTTTGTCATAGC |
| | CAGCAAAAGGATAAAGTATCTCGGAATCCAGCTGACACGAGACGTTAAAGATTTGTTTAAGGAAAAT |
| | TACAAGCCTCTCCTGAAAGAGATTAAGGAAGATACTAATAAGTGGAAGAATATCCCCTGTTCATGGG |
| | TTGGCAGAATCAACATAGTGAAGATGGCAATACTTCCTAAAGTGATATATCGCTTTAACGCCATCCC |
| | AATTAAACTGCCTATGACCTTCTTTACGGAGCTCGAGAAAACAACCCTTAAATTTATATGGAATCAA |
| | AAGAGAGCAAGAATAGCGAAGTCCATCTTGAGCCAGAAGAATAAGGCCGGTGGGATTACTTTGCCTG |
| | ATTTTAAGTTGTATTATAAAGCCACAGTAACTAAGACAGCCTGGTATTGGTATCAGAATAGAGACAT |
| | CGAGCAGTGGAATCGGACCGAACCATCAGAGATAATGCCCCACATCTATAATTACCTTATATTCGAT |
| | AAGCCAGAAAAGAATAAACAGTGGGGCAAAGACAGCCTCTTCAACAAGTGGTGTTGGGAGAATTGGC |
| | TGGCCATATGCCGGAAACTCAAGCTCGACCCCTTTCTTACACCCTACACTAAAATCAACAGTAGGTG |
| | GATCAAGGACTTGAATGTCAAGCCAAAGACTATAAAGACACTGGAAGAGAATCTTGGGATCACAATA |
| | CAAGATATAGGCGTCGGCAAAGATTTTATGTCAAAGACGCCCAAGGCCATGGCCACTAAGGATAAGA |
| | TTGATAAGTGGGACCTTATTAAGCTCAAAAGCTTCTGTACTGCCAAGGAGACCACGATCAGAGTTAA |
| | TAGGCAGCCCACTACATGGGAAAAGATTTTCGCCACTTATTCATCAGATAAGGGGTTGATAAGCAGA |
| | ATATATAACGAGCTGAAGCAGATCTACAAGAAGAAAACGAATAATCCCATCAAGAAGTGGGCAAAAG |
| | ATATGAACAGGCATTTTAGCAAAGAGGATATCTACGCCGCGAAGAAGCATATGAAGAAGTGTAGTTC |
| | AAGCTTGGCCATTCGTGAGATGCAGATTAAGACGACCATGCGATACCACCTTACCCCAGTGAGGATG |
| | GCAATTATCAAGAAATCTGGCAATAATAGATGTTGGCGGGGCTGTGGCGAGATTGGCACCCTGCTCC |
| | ATTGCTGGTGGGATTGCAAGCTGGTGCAGCCGCTTTGGAAATCAGTCTGGCGCTTTCTGAGGGACCT |
| | CGAGCTTGAGATTCCCTTCGATCCCGCAATTCCCTTGCTCGGAATCTATCCTAACGAATACAAGAGC |
| | TGTTGTTACAAGGATACGTGTACCCGGATGTTCATCGCGGCCTTGTTTACGATAGCTAAGACGTGGA |
| | ATCAGCCTAAGTGCCCCACAATGATCGATTGGATCAAGAAAATGTGGCATATTTATACCATGGAGTA |
| | TTACGCAGCAATTAAGAATGACGAATTTATTTCCTTCGTTGGGACCTGGATGAAGCTGGAGACTATT |
| | ATTCTGAGCAAGCTGTCTCAGGAGCAAAAGACAAAGCATAGAATCTTCTCTCTCATTGGTGGTAACG |
| | ACTACAAAGACGATGACGACAAGTAAAGCGCTTCTAGAAGTTGTCTCCTCCTGCACTGACTGACTGA |
| | TACAATCGATTTCTGGATCCGCAGGCCTAATCAACCTCTGGATTACAAAATTTGTGAAAGATTGACT |
| | GGTATTCTTAACTATGTTGCTCCTTTTACGCTATGTGGATACGCTGCTTTAATGCCTTTGTATCATG |
| | CTATTGCTTCCCGTATGGCTTTCATTTTCTCCTCCTTGTATAAATCCTGGTTGCTGTCTCTTTATGA |
| | GGAGTTGTGGCCCGTTGTCAGGCAACGTGGCGTGGTGTGCACTGTGTTTGCTGACGCAACCCCCACT |
| | GGTTGGGGCATTGCCACCACCTGTCAGCTCCTTTCCGGGACTTTCGCTTTCCCCCTCCCTATTGCCA |
| | CGGCGGAACTCATCGCCGCCTGCCTTGCCCGCTGCTGGACAGGGGCTCGGCTGTTGGGCACTGACAA |
| | TTCCGTGGTGTTGTCGGGGAAGCTGACGTCCTTTCCATGGCTGCTCGCCTGTGTTGCCACCTGGATT |
| | CTGCGCGGGACGTCCTTCTGCTACGTCCCTTCGGCCCTCAATCCAGCGGACCTTCCTTCCCGCtgag |
| | agacacaaaaaattccaacacactattgcaatgaaaataaatttcctttattagccagaagtcagat |
| | gctcaaggggcttcatgatgtccccataatttttggcagagggaaaaagatctcagtggtatttgtg |
| | agccagggcattggccttctgataggcagcctgcacctgaggagtgcggccgctttacttgtacagc |
| | tcgtccatgccgagagtgatcccggcggcggtcacgaactccagcaggaccatgtgatcgcgcttct |
| | cgttggggtctttgctcagggcggactgggtgctcaggtagtggttgtcgggcagcagcacggggcc |
| | gtcgccgatgggggtgttctgctggtagtggtcggcgagctgcacgctgccgtcctcgatgttgtgg |
| | cggatcttgaagttcaccttgatgccgttcttctgcttgtcggccatgatatagacgttgtggctgt |
| | tgtagttgtactccagcttgtgccccaggatgttgccgtcctccttgaagtcgatgcccttcagctc |
| | gatgcggttcaccagggtgtcgccctcgaacttcacctcggcgcgggtcttgtagttgccgtcgtcc |
| | ttgaagaagatggtgcgctcctggacgtagccttcgggcatggcggacttgaagaagtcgtgctgct |
| | tcatgtggtcggggtagcggctgaagcactgcacgccgtaggtcagggtggtcacgagggtgggcca |
| | gggcacgggcagcttgccggtggtgcagatgaacttcagggtcagcttgccgtaggtggcatcgccc |
| | tcgccctcgccggacacgctgaacttgtggccgtttacgtcgccgtccagctcgaccaggatgggca |
| | ccaccccggtgaacagctcctcgcccttgctcaccatggtggcgggatctgacggttcactaaacca |
| | gctctgcttatatagacctcccaccgtacacgcctaccgcccatttgcgtcaatggggcggagttgt |
| | tacgacattttggaaagtcccgttgattttggtgccaaaacaaactcccattgacgtcaatggggtg |
| | gagacttggaaatccccgtgagtcaaaccgctatccacgcccattgatgtactgccaaaaccgcatc |
| | accatggtaatagcgatgactaatacgtagatgtactgccaagtaggaaagtcccataaggtcatgt |
| | actgggcataatgccaggcgggccatttaccgtcattgacgtcaatagggggcgtacttggcatatg |
| | atacacttgatgtactgccaagtgggcagtttaccgtaaatactccacccattgacgtcaatggaaa |
| | gtccctattggcgttactatgggaacatacgtcattattgacgtcaatgggcgggggtcgttgggcg |
| | gtcagccaggcgggccatttaccgtaagttatgtaacgGGCCTGCTGCCGGCTCTGCGGCCTCTTCC |
| | GCGTCTTCGCCTTCGCCCTCAGACGAGTCGGATCTCCCTTTGGGCCGCCTCCCCGCCTGTCTAGCTT |
| | GACTGACTGAGATACAGCGTACCTTCAGCTCACAGACATGATAAGATACATTGATGAGTTTGGACAA |
| | ACCACAACTAGAATGCAGTGAAAAAAATGCTTTATTTGTGAAATTTGTGATGCTATTGCTTTATTTG |
| | TAACCATTATAAGCTGCAATAAACAAGTTAACAACAACAATTGCATTCATTTTATGTTTCAGGTTCA |
| | GGGGGAGGTGTGGGAGGTTTTTTAAAGCAAGTAAAACCTCTACAAATGTGGTATTGGCCCATCTCTA |
| | TCGGTATCGTAGCATAACCCCTTGGGGCCTCTAAACGGGTCTTGAGGGGTTTTTTGTGCCCCTCGGG |
| | CCGGATTGCTATCTACCGGCATTGGCGCAGAAAAAAATGCCTGATGCGACGCTGCGCGTCTTATACT |
| | CCCACATATGCCAGATTCAGCAACGGATACGGCTTCCCCAACTTGCCCACTTCCATACGTGTCCTCC |
| | TTACCAGAAATTTATCCTTAAGGTCGTCAGCTATCCTGCAGGCGATCTCTCGATTTCGATCAAGACA |
| | TTCCTTTAATGGTCTTTTCTGGACACCACTAGGGGTCAGAAGTAGTTCATCAAACTTTCTTCCCTCC |
| | CTAATCTCATTGGTTACCTTGGGCTATCGAAACTTAATTAAGCGATCTGCATCTCAATTAGTCAGCA |
| | ACCATAGTCCCGCCCCTAACTCCGCCCATCCCGCCCCTAACTCCGCCCAGTTCCGCCCATTCTCCGC |
| | CCCATCGCTGACTAATTTTTTTTATTTATGCAGAGGCCGAGGCCGCCTCGGCCTCTGAGCTATTCCA |
| | GAAGTAGTGAGGAGGCTTTTTTGGAGGCCTAGGCTTTTGCAAAGGAGGTAGCCAACATGATTGAACA |
| | AGATGGATTGCACGCAGGTTCTCCCGCCGCTTGGGTGGAGAGGCTATTCGGCTATGACTGGGCACAA |
| | CAGACAATCGGCTGCTCTGATGCCGCCGTGTTCCGGCTGTCAGCGCAGGGGCGCCCGGTTCTTTTTG |
| | TCAAGACCGACCTGTCCGGTGCCCTGAATGAACTCCAGGACGAGGCAGCGCGGCTATCGTGGCTGGC |
| | CACGACGGGCGTTCCTTGCGCAGCTGTGCTCGACGTTGTCACTGAAGCGGGAAGGGACTGGCTGCTA |
| | TTGGGCGAAGTGCCGGGGCAGGATCTCCTGTCATCTCACCTTGCTCCTGCCGAGAAAGTATCCATCA |
| | TGGCTGATGCAATGCGGCGGCTGCATACGCTTGATCCGGCTACCTGCCCATTCGACCACCAAGCGAA |
| | ACATCGCATCGAGCGAGCACGTACTCGGATGGAAGCCGGTCTTGTCGATCAGGATGATCTGGACGAA |
| | GAGCATCAGGGGCTCGCGCCAGCCGAACTGTTCGCCAGGCTCAAGGCGCGGATGCCCGACGGCGAGG |
| | ATCTCGTCGTGACCCACGGCGATGCCTGCTTGCCGAATATCATGGTGGAAAATGGCCGCTTTTCTGG |
| | ATTCATCGACTGTGGCCGGCTGGGTGTGGCGGACCGCTATCAGGACATAGCGTTGGCTACCCGTGAT |
| | ATTGCTGAAGAGCTTGGCGGCGAATGGGCTGACCGCTTCCTCGTGCTTTACGGTATCGCCGCTCCCG |
| | ATTCGCAGCGCATCGCCTTCTATCGCCTTCTTGACGAGTTCTTCTAGTATGTAAGCCCTGTGCCTTC |
| | TAGTTGCCAGCCATCTGTTGTTTGCCCCTCCCCCGTGCCTTCCTTGACCCTGGAAGGTGCCACTCCC |
| | ACTGTCCTTTCCTAATAAAATGAGGAAATTGCATCGCATTGTCTGAGTAGGTGTCATTCTATTCTGG |
| | GGGGTGGGGTGGGGCAGGACAGCAAGGGGGAGGATTGGGAAGACAATAGCAGGCATGCTGGGGATGC |
| | GGTGGGCTCTATGGTTAATTAACCAGTCAAGTCAGCTACTTGGCGAGATCGACTTGTCTGGGTTTCG |
| | ACTACGCTCAGAATTGCGTCAGTCAAGTTCGATCTGGTCCTTGCTATTGCACCCGTTCTCCGATTAC |
| | GAGTTTCATTTAAATCATGTGAGCAAAAGGCCAGCAAAAGGCCAGGAACCGTAAAAAGGCCGCGTTG |
| | CTGGCGTTTTTCCATAGGCTCCGCCCCCCTGACGAGCATCACAAAAATCGACGCTCAAGTCAGAGGT |
| | GGCGAAACCCGACAGGACTATAAAGATACCAGGCGTTTCCCCCTGGAAGCTCCCTCGTGCGCTCTCC |
| | TGTTCCGACCCTGCCGCTTACCGGATACCTGTCCGCCTTTCTCCCTTCGGGAAGCGTGGCGCTTTCT |
| | CATAGCTCACGCTGTAGGTATCTCAGTTCGGTGTAGGTCGTTCGCTCCAAGCTGGGCTGTGTGCACG |
| | AACCCCCCGTTCAGCCCGACCGCTGCGCCTTATCCGGTAACTATCGTCTTGAGTCCAACCCGGTAAG |
| | ACACGACTTATCGCCACTGGCAGCAGCCACTGGTAACAGGATTAGCAGAGCGAGGTATGTAGGCGGT |
| | GCTACAGAGTTCTTGAAGTGGTGGCCTAACTACGGCTACACTAGAAGAACAGTATTTGGTATCTGCG |
| | CTCTGCTGAAGCCAGTTACCTTCGGAAAAAGAGTTGGTAGCTCTTGATCCGGCAAACAAACCACCGC |
| | TGGTAGCGGTGGTTTTTTTGTTTGCAAGCAGCAGATTACGCGCAGAAAAAAAGGATCTCAAGAAGAT |
| | CCTTTGATCTTTTCTACGGGGTCTGACGCTCAGTGGAACGAAAACTCACGTTAAGGGATTTTGGTCA |
| | TGAGATTATCAAAAAGGATCTTCACCTAGATCCTTTTAAATTAAAAATGAAGTTTTAAATCAATCTA |
| | AAGTATATATGAGTAAACTTGGTCTGACAGTTACCAATGCTTAATCAGTGAGGCACCTATCTCAGCG |
| | ATCTGTCTATTTCGTTCATCCATAGTTGCATTTAAATTTCCGAACTCTCCAAGGCCCTCGTCGGAAA |
| | ATCTTCAAACCTTTCGTCCGATCCATCTTGCAGGCTACCTCTCGAACGAACTATCGCAAGTCTCTTG |
| | GCCGGCCTTGCGCCTTGGCTATTGCTTGGCAGCGCCTATCGCCAGGTATTACTCCAATCCCGAATAT |
| | CCGAGATCGGGATCACCCGAGAGAAGTTCAACCTACATCCTCAATCCCGATCTATCCGAGATCCGAG |
| | GAATATCGAAATC (SEQ ID NO: 100) |
| ret-011- | TGGCCGGCCTTGCGCCTTGGCTATTGCTTGGCAGCGCCTATCGCCAGGTATTACTCCAATCCCGAAT |
| line1- | ATCCGAGATCGGGATCACCCGAGAGAAGTTCAACCTACATCCTCAATCCCGATCTATCCGAGATCCG |
| gfp- | AGGAATATCGAAATCGGGGCGCGCCTGGTGTACCGAGAACGATCCTCTCAGTGCGAGTCTCGACGAT |
| orf2-n- | CCATATCGTTGCTTGGCAGTCAGCCAGTCGGAATCCAGCTTGGGACCCAGGAAGTCCAATCGTCAGA |
| sv40- | TATTGTACTCAAGCCTGGTCACGGCAGCGTACCGATCTGTTTAAACCTAGATATTGATAGTCTGATC |
| nls- | GGTCAACGTATAATCGAGTCCTAGCTTTTGCAAACATCTATCAAGAGACAGGATCAGCAGGAGGCTT |
| linker | TCGCATGAGTATTCAACATTTCCGTGTCGCCCTTATTCCCTTTTTTGCGGCATTTTGCCTTCCTGTT |
| | TTTGCTCACCCAGAAACGCTGGTGAAAGTAAAAGATGCTGAAGATCAGTTGGGTGCGCGAGTGGGTT |
| | ACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAA |
| | CTCGGTCGCCGCATACACTATTCTCAGAATGACTTGGTTGAGTATTCACCAGTCACAGAAAAGCATC |
| | TTACGGATGGCATGACAGTAAGAGAATTATGCAGTGCTGCCATAACCATGAGTGATAACACTGCGGC |
| | CAACTTACTTCTGACAACGATTGGAGGACCGAAGGAGCTAACCGCTTTTTTGCACAACATGGGGGAT |
| | CATGTAACTCGCCTTGATCGTTGGGAACCGGAGCTGAATGAAGCCATACCAAACGACGAGCGTGACA |
| | CCACGATGCCTGTAGCAATGGCAACAACCTTGCGTAAACTATTAACTGGCGAACTACTTACTCTAGC |
| | TTCCCGGCAACAGTTGATAGACTGGATGGAGGCGGATAAAGTTGCAGGACCACTTCTGCGCTCGGCC |
| | CTTCCGGCTGGCTGGTTTATTGCTGATAAATCTGGAGCCGGTGAGCGTGGGTCTCGCGGTATCATTG |
| | CAGCACTGGGGCCAGATGGTAAGCCCTCCCGTATCGTAGTTATCTACACGACGGGGAGTCAGGCAAC |
| | TATGGATGAACGAAATAGACAGATCGCTGAGATAGGTGCCTCACTGATTAAGCATTGGTAACCGATT |
| | CTAGGTGCATTGGCGCAGAAAAAAATGCCTGATGCGACGCTGCGCGTCTTATACTCCCACATATGCC |
| | AGATTCAGCAACGGATACGGCTTCCCCAACTTGCCCACTTCCATACGTGTCCTCCTTACCAGAAATT |
| | TATCCTTAAGATCGTTTAAACTCGACTCTGGCTCTATCGAATCTCCGTCGTTTCGAGCTTACGCGAA |
| | CAGCCGTGGCGCTCATTTGCTCGTCGGGCATCGAATCTCGTCAGCTATCGTCAGCTTACCTTTTTGG |
| | CAGCGATCGCGGCTCCCGACATCTTGGACCATTAGCTCCACAGGTATCTTCTTCCCTCTAGTGGTCA |
| | TAACAGCAGCTTCAGCTACCTCTCAATTCAAAAAACCCCTCAAGACCCGTTTAGAGGCCCCAAGGGG |
| | TTATGCTATCAATCGTTGCGTTACACACACAAAAAACCAACACACATCCATCTTCGATGGATAGCGA |
| | TTTTATTATCTAACTGCTGATCGAGTGTAGCCAGATCTAGTAATCAATTACGGGGTCATTAGTTCAT |
| | AGCCCATATATGGAGTTCCGCGTTACATAACTTACGGTAAATGGCCCGCCTGGCTGACCGCCCAACG |
| | ACCCCCGCCCATTGACGTCAATAATGACGTATGTTCCCATAGTAACGCCAATAGGGACTTTCCATTG |
| | ACGTCAATGGGTGGAGTATTTACGGTAAACTGCCCACTTGGCAGTACATCAAGTGTATCATATGCCA |
| | AGTACGCCCCCTATTGACGTCAATGACGGTAAATGGCCCGCCTGGCATTATGCCCAGTACATGACCT |
| | TATGGGACTTTCCTACTTGGCAGTACATCTACGTATTAGTCATCGCTATTACCATGCTGATGCGGTT |
| | TTGGCAGTACATCAATGGGCGTGGATAGCGGTTTGACTCACGGGGATTTCCAAGTCTCCACCCCATT |
| | GACGTCAATGGGAGTTTGTTTTGGCACCAAAATCAACGGGACTTTCCAAAATGTCGTAACAACTCCG |
| | CCCCATTGACGCAAATGGGCGGTAGGCGTGTACGGTGGGAGGTCTATATAAGCAGAGCTGGTTTAGT |
| | GAACCGTCAGATCAGATCTTTGTCGATCCTACCATCCACTCGACACACCCGCCAGCGGCCGCTAATA |
| | CGACTCACTATAGGGAGAAGTACTGCCACCATGGGCAAGAAGCAAAATCGCAAGACGGGGAATTCCA |
| | AGACACAATCCGCTAGCCCACCACCTAAAGAGCGTTCTAGCTCCCCTGCTACTGAGCAGTCCTGGAT |
| | GGAAAACGACTTCGATGAACTCCGGGAAGAGGGATTTAGGCGATCCAACTATTCAGAACTCCGCGAA |
| | GATATCCAGACAAAGGGGAAGGAAGTCGAGAATTTCGAGAAGAACCTCGAGGAGTGCATCACCCGTA |
| | TCACAAACACTGAGAAATGTCTCAAAGAACTCATGGAACTTAAGACAAAAGCCAGGGAGCTTCGAGA |
| | GGAGTGTCGGAGTCTGAGATCCAGGTGTGACCAGCTCGAGGAGCGCGTGAGCGCGATGGAAGACGAG |
| | ATGAACGAGATGAAAAGAGAGGGCAAATTCAGGGAGAAGCGCATTAAGAGGAACGAACAGAGTCTGC |
| | AGGAGATTTGGGATTACGTCAAGAGGCCTAACCTGCGGTTGATCGGCGTCCCCGAGAGCGACGTAGA |
| | AAACGGGACTAAACTGGAGAATACACTTCAAGACATCATTCAAGAAAATTTTCCAAACCTGGCTCGG |
| | CAAGCTAATGTGCAAATCCAAGAGATCCAACGCACACCCCAGCGGTATAGCTCTCGGCGTGCCACCC |
| | CTAGGCATATTATCGTGCGCTTTACTAAGGTGGAGATGAAAGAGAAGATGCTGCGAGCCGCTCGGGA |
| | AAAGGGAAGGGTGACTTTGAAGGGCAAACCTATTCGGCTGACGGTTGACCTTAGCGCCGAGACACTC |
| | CAGGCACGCCGGGAATGGGGCCCCATCTTTAATATCCTGAAGGAGAAGAACTTCCAGCCACGAATCT |
| | CTTACCCTGCAAAGTTGAGTTTTATCTCCGAGGGTGAGATTAAGTATTTCATCGATAAACAGATGCT |
| | GCGAGACTTCGTGACAACTCGCCCAGCTCTCAAGGAACTGCTCAAAGAGGCTCTTAATATGGAGCGC |
| | AATAATAGATATCAACCCTTGCAGAACCACGCAAAGATGTGAGACAGCCGTCAGACCATCAAGACTA |
| | GGAAGAAACTGCATCAACTAATGAGCAAAATCACCAGCTAACATCATAGTATACATGccaaagaaga |
| | agcggaaggtcggcggcggcagcACCGGCTCTAACTCACATATCACCATCCTTACACTTAACATTAA |
| | CGGCCTCAACTCAGCTATCAAGCGCCATCGGCTGGCCAGCTGGATCAAATCACAGGATCCAAGCGTT |
| | TGTTGCATCCAAGAGACCCACCTGACCTGTAGAGATACTCACCGCCTCAAGATCAAGGGATGGCGAA |
| | AGATTTATCAGGCGAACGGTAAGCAGAAGAAAGCCGGAGTCGCAATTCTGGTCTCAGACAAGACGGA |
| | TTTCAAGCCCACCAAAATTAAGCGTGATAAGGAAGGTCACTATATTATGGTGAAAGGCAGCATACAG |
| | CAGGAAGAACTTACCATATTGAACATCTACGCGCCAAACACCGGCGCACCTCGCTTTATCAAACAGG |
| | TCCTGTCCGATCTGCAGCGAGATCTGGATTCTCATACGTTGATTATGGGTGATTTCAATACACCATT |
| | GAGCACCCTGGATCGCAGCACCAGGCAAAAGGTAAATAAAGACACGCAAGAGCTCAATAGCGCACTG |
| | CATCAGGCAGATCTCATTGATATTTATCGCACTCTTCATCCTAAGAGTACCGAGTACACATTCTTCA |
| | GCGCCCCACATCATACATACTCAAAGATCGATCATATCGTCGGCTCAAAGGCTCTGCTGTCAAAGTG |
| | CAAGCGCACAGAGATAATTACAAATTACCTGTCAGATCATAGCGCGATCAAGCTCGAGCTGAGAATC |
| | AAGAACCTGACCCAGAGCCGGAGTACCACTTGGAAGCTTAATAACCTGCTGCTCAACGATTATTGGG |
| | TCCACAATGAGATGAAGGCAGAGATTAAAATGTTCTTCGAAACAAATGAGAATAAGGATACTACCTA |
| | TCAAAACCTTTGGGATGCCTTTAAGGCCGTCTGCAGAGGCAAGTTCATCGCCCTCAACGCCTATAAA |
| | AGAAAACAAGAGAGATCTAAGATCGATACTCTCACCTCTCAGCTGAAGGAGTTGGAGAAACAGGAAC |
| | AGACCCACTCCAAGGCGTCAAGACGGCAGGAGATCACAAAGATTCGCGCCGAGTTGAAAGAGATCGA |
| | AACCCAAAAGACTCTTCAGAAAATTAACGAGTCTCGTAGTTGGTTCTTCGAGCGGATTAATAAGATA |
| | GACAGACCTCTGGCACGACTGATTAAGAAGAAGCGCGAAAAGAACCAGATTGATACCATCAAGAACG |
| | ACAAGGGCGACATCACTACTGACCCGACCGAGATCCAGACCACTATTCGGGAGTATTATAAGCATTT |
| | GTATGCTAACAAGCTTGAGAACCTGGAAGAGATGGACACTTTTCTGGATACCTATACTCTGCCACGG |
| | CTTAATCAAGAGGAAGTCGAGTCCCTCAACCGCCCAATTACAGGAAGCGAGATTGTGGCCATAATTA |
| | ACTCCCTGCCGACAAAGAAATCTCCTGGTCCGGACGGGTTTACAGCTGAGTTTTATCAACGGTATAT |
| | GGAAGAGCTTGTACCGTTTCTGCTCAAGCTCTTTCAGTCTATAGAAAAGGAAGGCATCTTGCCCAAT |
| | TCCTTCTACGAAGCTTCTATAATACTTATTCCCAAACCAGGACGCGATACCACAAAGAAGGAAAACT |
| | TCCGGCCCATTAGTCTCATGAATATCGACGCTAAAATATTGAACAAGATTCTCGCCAACAGAATCCA |
| | ACAACATATTAAGAAATTGATACATCACGACCAGGTGGGGTTTATACCTGGCATGCAGGGCTGGTTT |
| | AACATCCGGAAGAGTATTAACGTCATTCAACACATTAATAGAGCTAAGGATAAGAATCATATGATCA |
| | TCTCTATAGACGCGGAAAAGGCATTCGATAAGATTCAGCAGCCATTTATGCTCAAGACTCTGAACAA |
| | ACTCGGCATCGACGGAACATATTTTAAGATTATTCGCGCAATTTACGATAAGCCGACTGCTAACATT |
| | ATCCTTAACGGCCAAAAGCTCGAGGCCTTTCCGCTCAAGACTGGAACCCGCCAAGGCTGTCCCCTCT |
| | CCCCGCTTTTGTTTAATATTGTACTCGAGGTGCTGGCTAGGGCTATTCGTCAAGAGAAAGAGATTAA |
| | AGGGATACAGCTCGGGAAGGAAGAGGTCAAGCTTTCCTTGTTCGCCGATGATATGATTGTGTACCTG |
| | GAGAATCCTATTGTGTCTGCTCAGAACCTTCTTAAACTTATTTCTAACTTTAGCAAGGTCAGCGGCT |
| | ATAAGATTAACGTCCAGAAATCTCAGGCCTTTCTGTACACAAATAATCGACAGACCGAATCCCAGAT |
| | AATGGGTGAGCTTCCGTTTGTCATAGCCAGCAAAAGGATAAAGTATCTCGGAATCCAGCTGACACGA |
| | GACGTTAAAGATTTGTTTAAGGAAAATTACAAGCCTCTCCTGAAAGAGATTAAGGAAGATACTAATA |
| | AGTGGAAGAATATCCCCTGTTCATGGGTTGGCAGAATCAACATAGTGAAGATGGCAATACTTCCTAA |
| | AGTGATATATCGCTTTAACGCCATCCCAATTAAACTGCCTATGACCTTCTTTACGGAGCTCGAGAAA |
| | ACAACCCTTAAATTTATATGGAATCAAAAGAGAGCAAGAATAGCGAAGTCCATCTTGAGCCAGAAGA |
| | ATAAGGCCGGTGGGATTACTTTGCCTGATTTTAAGTTGTATTATAAAGCCACAGTAACTAAGACAGC |
| | CTGGTATTGGTATCAGAATAGAGACATCGACCAGTGGAATCGGACCGAACCATCAGAGATAATGCCC |
| | CACATCTATAATTACCTTATATTCGATAAGCCAGAAAAGAATAAACAGTGGGGCAAAGACAGCCTCT |
| | TCAACAAGTGGTGTTGGGAGAATTGGCTGGCCATATGCCGGAAACTCAAGCTCGACCCCTTTCTTAC |
| | ACCCTACACTAAAATCAACAGTAGGTGGATCAAGGACTTGAATGTCAAGCCAAAGACTATAAAGACA |
| | CTGGAAGAGAATCTTGGGATCACAATACAAGATATAGGCGTCGGCAAAGATTTTATGTCAAAGACGC |
| | CCAAGGCCATGGCCACTAAGGATAAGATTGATAAGTGGGACCTTATTAAGCTCAAAAGCTTCTGTAC |
| | TGCCAAGGAGACCACGATCAGAGTTAATAGGCAGCCCACTACATGGGAAAAGATTTTCGCCACTTAT |
| | TCATCAGATAAGGGGTTGATAAGCAGAATATATAACGAGCTGAAGCAGATCTACAAGAAGAAAACGA |
| | ATAATCCCATCAAGAAGTGGGCAAAAGATATGAACAGGCATTTTAGCAAAGAGGATATCTACGCCGC |
| | GAAGAAGCATATGAAGAAGTGTAGTTCAAGCTTGGCCATTCGTGAGATGCAGATTAAGACGACCATG |
| | CGATACCACCTTACCCCAGTGAGGATGGCAATTATCAAGAAATCTGGCAATAATAGATGTTGGCGGG |
| | GCTGTGGCGAGATTGGCACCCTGCTCCATTGCTGGTGGGATTGCAAGCTGGTGCAGCCGCTTTGGAA |
| | ATCAGTCTGGCGCTTTCTGAGGGACCTCGAGCTTGAGATTCCCTTCGATCCCGCAATTCCCTTGCTC |
| | GGAATCTATCCTAACGAATACAAGAGCTGTTGTTACAAGGATACGTGTACCCGGATGTTCATCGCGG |
| | CCTTGTTTACGATAGCTAAGACGTGGAATCAGCCTAAGTGCCCCACAATGATCGATTGGATCAAGAA |
| | AATGTGGCATATTTATACCATGGAGTATTACGCAGCAATTAAGAATGACGAATTTATTTCCTTCGTT |
| | GGGACCTGGATGAAGCTGGAGACTATTATTCTGAGCAAGCTGTCTCAGGAGCAAAAGACAAAGCATA |
| | GAATCTTCTCTCTCATTGGTGGTAACGACTACAAAGACGATGACGACAAGTAAAGCGCTTCTAGAAG |
| | TTGTCTCCTCCTGCACTGACTGACTGATACAATCGATTTCTGGATCCGCAGGCCTAATCAACCTCTG |
| | GATTACAAAATTTGTGAAAGATTGACTGGTATTCTTAACTATGTTGCTCCTTTTACGCTATGTGGAT |
| | ACGCTGCTTTAATGCCTTTGTATCATGCTATTGCTTCCCGTATGGCTTTCATTTTCTCCTCCTTGTA |
| | TAAATCCTGGTTGCTGTCTCTTTATGAGGAGTTGTGGCCCGTTGTCAGGCAACGTGGCGTGGTGTGC |
| | ACTGTGTTTGCTGACGCAACCCCCACTGGTTGGGGCATTGCCACCACCTGTCAGCTCCTTTCCGGGA |
| | CTTTCGCTTTCCCCCTCCCTATTGCCACGGCGGAACTCATCGCCGCCTGCCTTGCCCGCTGCTGGAC |
| | AGGGGCTCGGCTGTTGGGCACTGACAATTCCGTGGTGTTGTCGGGGAAGCTGACGTCCTTTCCATGG |
| | CTGCTCGCCTGTGTTGCCACCTGGATTCTGCGCGGGACGTCCTTCTGCTACGTCCCTTCGGCCCTCA |
| | ATCCAGCGGACCTTCCTTCCCGCtgagagacacaaaaaattccaacacactattgcaatgaaaataa |
| | atttcctttattagccagaagtcagatgctcaaggggcttcatgatgtccccataatttttggcaga |
| | gggaaaaagatctcagtggtatttgtgagccagggcattggccttctgataggcagcctgcacctga |
| | ggagtgcggccgctttacttgtacagctcgtccatgccgagagtgatcccggcggcggtcacgaact |
| | ccagcaggaccatgtgatcgcgcttctcgttggggtctttgctcagggcggactgggtgctcaggta |
| | gtggttgtcgggcagcagcacggggccgtcgccgatgggggtgttctgctggtagtggtcggcgagc |
| | tgcacgctgccgtcctcgatgttgtggcggatcttgaagttcaccttgatgccgttcttctgcttgt |
| | cggccatgatatagacgttgtggctgttgtagttgtactccagcttgtgccccaggatgttgccgtc |
| | ctccttgaagtcgatgcccttcagctcgatgcggttcaccagggtgtcgccctcgaacttcacctcg |
| | gcgcgggtcttgtagttgccgtcgtccttgaagaagatggtgcgctcctggacgtagccttcgggca |
| | tggcggacttgaagaagtcgtgctgcttcatgtggtcggggtagcggctgaagcactgcacgccgta |
| | ggtcagggtggtcacgagggtgggccagggcacgggcagcttgccggtggtgcagatgaacttcagg |
| | gtcagcttgccgtaggtggcatcgccctcgccctcgccggacacgctgaacttgtggccgtttacgt |
| | cgccgtccagctcgaccaggatgggcaccaccccggtgaacagctcctcgcccttgctcaccatggt |
| | ggcgggatctgacggttcactaaaccagctctgcttatatagacctcccaccgtacacgcctaccgc |
| | ccatttgcgtcaatggggcggagttgttacgacattttggaaagtcccgttgattttggtgccaaaa |
| | caaactcccattgacgtcaatggggtggagacttggaaatccccgtgagtcaaaccgctatccacgc |
| | ccattgatgtactgccaaaaccgcatcaccatggtaatagcgatgactaatacgtagatgtactgcc |
| | aagtaggaaagtcccataaggtcatgtactgggcataatgccaggcgggccatttaccgtcattgac |
| | gtcaatagggggcgtacttggcatatgatacacttgatgtactgccaagtgggcagtttaccgtaaa |
| | tactccacccattgacgtcaatggaaagtccctattggcgttactatgggaacatacgtcattattg |
| | acgtcaatgggcgggggtcgttgggcggtcagccaggcgggccatttaccgtaagttatgtaacgGG |
| | CCTGCTGCCGGCTCTGCGGCCTCTTCCGCGTCTTCGCCTTCGCCCTCAGACGAGTCGGATCTCCCTT |
| | TGGGCCGCCTCCCCGCCTGTCTAGCTTGACTGACTGAGATACAGCGTACCTTCAGCTCACAGACATG |
| | ATAAGATACATTGATGAGTTTGGACAAACCACAACTAGAATGCAGTGAAAAAAATGCTTTATTTGTG |
| | AAATTTGTGATGCTATTGCTTTATTTGTAACCATTATAAGCTGCAATAAACAAGTTAACAACAACAA |
| | TTGCATTCATTTTATGTTTCAGGTTCAGGGGGAGGTGTGGGAGGTTTTTTAAAGCAAGTAAAACCTC |
| | TACAAATGTGGTATTGGCCCATCTCTATCGGTATCGTAGCATAACCCCTTGGGGCCTCTAAACGGGT |
| | CTTGAGGGGTTTTTTGTGCCCCTCGGGCCGGATTGCTATCTACCGGCATTGGCGCAGAAAAAAATGC |
| | CTGATGCGACGCTGCGCGTCTTATACTCCCACATATGCCAGATTCAGCAACGGATACGGCTTCCCCA |
| | ACTTGCCCACTTCCATACGTGTCCTCCTTACCAGAAATTTATCCTTAAGGTCGTCAGCTATCCTGCA |
| | GGCGATCTCTCGATTTCGATCAAGACATTCCTTTAATGGTCTTTTCTGGACACCACTAGGGGTCAGA |
| | AGTAGTTCATCAAACTTTCTTCCCTCCCTAATCTCATTGGTTACCTTGGGCTATCGAAACTTAATTA |
| | AGCGATCTGCATCTCAATTAGTCAGCAACCATAGTCCCGCCCCTAACTCCGCCCATCCCGCCCCTAA |
| | CTCCGCCCAGTTCCGCCCATTCTCCGCCCCATCGCTGACTAATTTTTTTTATTTATGCAGAGGCCGA |
| | GGCCGCCTCGGCCTCTGAGCTATTCCAGAAGTAGTGAGGAGGCTTTTTTGGAGGCCTAGGCTTTTGC |
| | AAAGGAGGTAGCCAACATGATTGAACAAGATGGATTGCACGCAGGTTCTCCCGCCGCTTGGGTGGAG |
| | AGGCTATTCGGCTATGACTGGGCACAACAGACAATCGGCTGCTCTGATGCCGCCGTGTTCCGGCTGT |
| | CAGCGCAGGGGCGCCCGGTTCTTTTTGTCAAGACCGACCTGTCCGGTGCCCTGAATGAACTCCAGGA |
| | CGAGGCAGCGCGGCTATCGTGGCTGGCCACGACGGGCGTTCCTTGCGCAGCTGTGCTCGACGTTGTC |
| | ACTGAAGCGGGAAGGGACTGGCTGCTATTGGGCGAAGTGCCGGGGCAGGATCTCCTGTCATCTCACC |
| | TTGCTCCTGCCGAGAAAGTATCCATCATGGCTGATGCAATGCGGCGGCTGCATACGCTTGATCCGGC |
| | TACCTGCCCATTCGACCACCAAGCGAAACATCGCATCGAGCGAGCACGTACTCGGATGGAAGCCGGT |
| | CTTGTCGATCAGGATGATCTGGACGAAGAGCATCAGGGGCTCGCGCCAGCCGAACTGTTCGCCAGGC |
| | TCAAGGCGCGGATGCCCGACGGCGAGGATCTCGTCGTGACCCACGGCGATGCCTGCTTGCCGAATAT |
| | CATGGTGGAAAATGGCCGCTTTTCTGGATTCATCGACTGTGGCCGGCTGGGTGTGGCGGACCGCTAT |
| | CAGGACATAGCGTTGGCTACCCGTGATATTGCTGAAGAGCTTGGCGGCGAATGGGCTGACCGCTTCC |
| | TCGTGCTTTACGGTATCGCCGCTCCCGATTCGCAGCGCATCGCCTTCTATCGCCTTCTTGACGAGTT |
| | CTTCTAGTATGTAAGCCCTGTGCCTTCTAGTTGCCAGCCATCTGTTGTTTGCCCCTCCCCCGTGCCT |
| | TCCTTGACCCTGGAAGGTGCCACTCCCACTGTCCTTTCCTAATAAAATGAGGAAATTGCATCGCATT |
| | GTCTGAGTAGGTGTCATTCTATTCTGGGGGGTGGGGTGGGGCAGGACAGCAAGGGGGAGGATTGGGA |
| | AGACAATAGCAGGCATGCTGGGGATGCGGTGGGCTCTATGGTTAATTAACCAGTCAAGTCAGCTACT |
| | TGGCGAGATCGACTTGTCTGGGTTTCGACTACGCTCAGAATTGCGTCAGTCAAGTTCGATCTGGTCC |
| | TTGCTATTGCACCCGTTCTCCGATTACGAGTTTCATTTAAATCATGTGAGCAAAAGGCCAGCAAAAG |
| | GCCAGGAACCGTAAAAAGGCCGCGTTGCTGGCGTTTTTCCATAGGCTCCGCCCCCCTGACGAGCATC |
| | ACAAAAATCGACGCTCAAGTCAGAGGTGGCGAAACCCGACAGGACTATAAAGATACCAGGCGTTTCC |
| | CCCTGGAAGCTCCCTCGTGCGCTCTCCTGTTCCGACCCTGCCGCTTACCGGATACCTGTCCGCCTTT |
| | CTCCCTTCGGGAAGCGTGGCGCTTTCTCATAGCTCACGCTGTAGGTATCTCAGTTCGGTGTAGGTCG |
| | TTCGCTCCAAGCTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCACTGGTAACAGG |
| | ATTAGCAGAGCGAGGTATGTAGGCGGTGCTACAGAGTTCTTGAAGTGGTGGCCTAACTACGGCTACA |
| | CTAGAAGAACAGTATTTGGTATCTGCGCTCTGCTGAAGCCAGTTACCTTCGGAAAAAGAGTTGGTAG |
| | CTCTTGATCCGGCAAACAAACCACCGCTGGTAGCGGTGGTTTTTTTGTTTGCAAGCAGCAGATTACG |
| | CGCAGAAAAAAAGGATCTCAAGAAGATCCTTTGATCTTTTCTACGGGGTCTGACGCTCAGTGGAACG |
| | AAAACTCACGTTAAGGGATTTTGGTCATGAGATTATCAAAAAGGATCTTCACCTAGATCCTTTTAAA |
| | TTAAAAATGAAGTTTTAAATCAATCTAAAGTATATATGAGTAAACTTGGTCTGACAGTTACCAATGC |
| | TTAATCAGTGAGGCACCTATCTCAGCGATCTGTCTATTTCGTTCATCCATAGTTGCATTTAAATTTC |
| | CGAACTCTCCAAGGCCCTCGTCGGAAAATCTTCAAACCTTTCGTCCGATCCATCTTGCAGGCTACCT |
| | CTCGAACGAACTATCGCAAGTCTCT (SEQ ID NO: 101) |
| RET- | GACGATCCATATCGTTGCTTGGCAGTCAGCCAGTCGGAATCCAGCTTGGGACCCAGGAAGTCCAATC |
| 012 | GTCAGATATTGTACTCAAGCCTGGTCACGGCAGCGTACCGATCTGTTTAAACCTAGATATTGATAGT |
| LINE1- | CTGATCGGTCAACGTATAATCGAGTCCTAGCTTTTGCAAACATCTATCAAGAGACAGGATCAGCAGG |
| GFP | AGGCTTTCGCATGAGTATTCAACATTTCCGTGTCGCCCTTATTCCCTTTTTTGCGGCATTTTGCCTT |
| ORF2-N | CCTGTTTTTGCTCACCCAGAAACGCTGGTGAAAGTAAAAGATGCTGAAGATCAGTTGGGTGCGCGAG |
| Nucleo- | TGGGTTACATCGAACTGGATCTCAACAGCGGTAAGATCCTTGAGAGTTTTCGCCCCGAAGAACGCTT |
| plasmin- | TCCAATGATGAGCACTTTTAAAGTTCTGCTATGTGGCGCGGTATTATCCCGTATTGACGCCGGGCAA |
| NLS | GAGCAACTCGGTCGCCGCATACACTATTCTCAGAATGACTTGGTTGAGTATTCACCAGTCACAGAAA |
| | AGCATCTTACGGATGGCATGACAGTAAGAGAATTATGCAGTGCTGCCATAACCATGAGTGATAACAC |
| | TGCGGCCAACTTACTTCTGACAACGATTGGAGGACCGAAGGAGCTAACCGCTTTTTTGCACAACATG |
| | GGGGATCATGTAACTCGCCTTGATCGTTGGGAACCGGAGCTGAATGAAGCCATACCAAACGACGAGC |
| | GTGACACCACGATGCCTGTAGCAATGGCAACAACCTTGCGTAAACTATTAACTGGCGAACTACTTAC |
| | TCTAGCTTCCCGGCAACAGTTGATAGACTGGATGGAGGCGGATAAAGTTGCAGGACCACTTCTGCGC |
| | TCGGCCCTTCCGGCTGGCTGGTTTATTGCTGATAAATCTGGAGCCGGTGAGCGTGGGTCTCGCGGTA |
| | TCATTGCAGCACTGGGGCCAGATGGTAAGCCCTCCCGTATCGTAGTTATCTACACGACGGGGAGTCA |
| | GGCAACTATGGATGAACGAAATAGACAGATCGCTGAGATAGGTGCCTCACTGATTAAGCATTGGTAA |
| | CCGATTCTAGGTGCATTGGCGCAGAAAAAAATGCCTGATGCGACGCTGCGCGTCTTATACTCCCACA |
| | TATGCCAGATTCAGCAACGGATACGGCTTCCCCAACTTGCCCACTTCCATACGTGTCCTCCTTACCA |
| | GAAATTTATCCTTAAGATCGTTTAAACTCGACTCTGGCTCTATCGAATCTCCGTCGTTTCGAGCTTA |
| | CGCGAACAGCCGTGGCGCTCATTTGCTCGTCGGGCATCGAATCTCGTCAGCTATCGTCAGCTTACCT |
| | TTTTGGCAGCGATCGCGGCTCCCGACATCTTGGACCATTAGCTCCACAGGTATCTTCTTCCCTCTAG |
| | TGGTCATAACAGCAGCTTCAGCTACCTCTCAATTCAAAAAACCCCTCAAGACCCGTTTAGAGGCCCC |
| | AAGGGGTTATGCTATCAATCGTTGCGTTACACACACAAAAAACCAACACACATCCATCTTCGATGGA |
| | TAGCGATTTTATTATCTAACTGCTGATCGAGTGTAGCCAGATCTAGTAATCAATTACGGGGTCATTA |
| | GTTCATAGCCCATATATGGAGTTCCGCGTTACATAACTTACGGTAAATGGCCCGCCTGGCTGACCGC |
| | CCAACGACCCCCGCCCATTGACGTCAATAATGACGTATGTTCCCATAGTAACGCCAATAGGGACTTT |
| | CCATTGACGTCAATGGGTGGAGTATTTACGGTAAACTGCCCACTTGGCAGTACATCAAGTGTATCAT |
| | ATGCCAAGTACGCCCCCTATTGACGTCAATGACGGTAAATGGCCCGCCTGGCATTATGCCCAGTACA |
| | TGACCTTATGGGACTTTCCTACTTGGCAGTACATCTACGTATTAGTCATCGCTATTACCATGCTGAT |
| | GCGGTTTTGGCAGTACATCAATGGGCGTGGATAGCGGTTTGACTCACGGGGATTTCCAAGTCTCCAC |
| | CCCATTGACGTCAATGGGAGTTTGTTTTGGCACCAAAATCAACGGGACTTTCCAAAATGTCGTAACA |
| | ACTCCGCCCCATTGACGCAAATGGGCGGTAGGCGTGTACGGTGGGAGGTCTATATAAGCAGAGCTGG |
| | TTTAGTGAACCGTCAGATCAGATCTTTGTCGATCCTACCATCCACTCGACACACCCGCCAGCGGCCG |
| | CTAATACGACTCACTATAGGGAGAAGTACTGCCACCATGGGCAAGAAGCAAAATCGCAAGACGGGGA |
| | ATTCCAAGACACAATCCGCTAGCCCACCACCTAAAGAGCGTTCTAGCTCCCCTGCTACTGAGCAGTC |
| | CTGGATGGAAAACGACTTCGATGAACTCCGGGAAGAGGGATTTAGGCGATCCAACTATTCAGAACTC |
| | CGCGAAGATATCCAGACAAAGGGGAAGGAAGTCGAGAATTTCGAGAAGAACCTCGAGGAGTGCATCA |
| | CCCGTATCACAAACACTGAGAAATGTCTCAAAGAACTCATGGAACTTAAGACAAAAGCCAGGGAGCT |
| | TCGAGAGGAGTGTCGGAGTCTGAGATCCAGGTGTGACCAGCTCGAGGAGCGCGTGAGCGCGATGGAA |
| | GACGAGATGAACGAGATGAAAAGAGAGGGCAAATTCAGGGAGAAGCGCATTAAGAGGAACGAACAGA |
| | GTCTGCAGGAGATTTGGGATTACGTCAAGAGGCCTAACCTGCGGTTGATCGGCGTCCCCGAGAGCGA |
| | CGTAGAAAACGGGACTAAACTGGAGAATACACTTCAAGACATCATTCAAGAAAATTTTCCAAACCTG |
| | GCTCGGCAAGCTAATGTGCAAATCCAAGAGATCCAACGCACACCCCAGCGGTATAGCTCTCGGCGTG |
| | CCACCCCTAGGCATATTATCGTGCGCTTTACTAAGGTGGAGATGAAAGAGAAGATGCTGCGAGCCGC |
| | TCGGGAAAAGGGAAGGGTGACTTTGAAGGGCAAACCTATTCGGCTGACGGTTGACCTTAGCGCCGAG |
| | ACACTCCAGGCACGCCGGGAATGGGGCCCCATCTTTAATATCCTGAAGGAGAAGAACTTCCAGCCAC |
| | GAATCTCTTACCCTGCAAAGTTGAGTTTTATCTCCGAGGGTGAGATTAAGTATTTCATCGATAAACA |
| | GATGCTGCGAGACTTCGTGACAACTCGCCCAGCTCTCAAGGAACTGCTCAAAGAGGCTCTTAATATG |
| | GAGCGCAATAATAGATATCAACCCTTGCAGAACCACGCAAAGATGTGAGACAGCCGTCAGACCATCA |
| | AGACTAGGAAGAAACTGCATCAACTAATGAGCAAAATCACCAGCTAACATCATAGTATACATGaaaa |
| | ggccggcggccacgaaaaaggccggccaggcaaaaaagaaaaagACCGGCTCTAACTCACATATCAC |
| | CATCCTTACACTTAACATTAACGGCCTCAACTCAGCTATCAAGCGCCATCGGCTGGCCAGCTGGATC |
| | AAATCACAGGATCCAAGCGTTTGTTGCATCCAAGAGACCCACCTGACCTGTAGAGATACTCACCGCC |
| | TCAAGATCAAGGGATGGCGAAAGATTTATCAGGCGAACGGTAAGCAGAAGAAAGCCGGAGTCGCAAT |
| | TCTGGTCTCAGACAAGACGGATTTCAAGCCCACCAAAATTAAGCGTGATAAGGAAGGTCACTATATT |
| | ATGGTGAAAGGCAGCATACAGCAGGAAGAACTTACCATATTGAACATCTACGCGCCAAACACCGGCG |
| | CACCTCGCTTTATCAAACAGGTCCTGTCCGATCTGCAGCGAGATCTGGATTCTCATACGTTGATTAT |
| | GGGTGATTTCAATACACCATTGAGCACCCTGGATCGCAGCACCAGGCAAAAGGTAAATAAAGACACG |
| | CAAGAGCTCAATAGCGCACTGCATCAGGCAGATCTCATTGATATTTATCGCACTCTTCATCCTAAGA |
| | GTACCGAGTACACATTCTTCAGCGCCCCACATCATACATACTCAAAGATCGATCATATCGTCGGCTC |
| | AAAGGCTCTGCTGTCAAAGTGCAAGCGCACAGAGATAATTACAAATTACCTGTCAGATCATAGCGCG |
| | ATCAAGCTCGAGCTGAGAATCAAGAACCTGACCCAGAGCCGGAGTACCACTTGGAAGCTTAATAACC |
| | TGCTGCTCAACGATTATTGGGTCCACAATGAGATGAAGGCAGAGATTAAAATGTTCTTCGAAACAAA |
| | TGAGAATAAGGATACTACCTATCAAAACCTTTGGGATGCCTTTAAGGCCGTCTGCAGAGGCAAGTTC |
| | ATCGCCCTCAACGCCTATAAAAGAAAACAAGAGAGATCTAAGATCGATACTCTCACCTCTCAGCTGA |
| | AGGAGTTGGAGAAACAGGAACAGACCCACTCCAAGGCGTCAAGACGGCAGGAGATCACAAAGATTCG |
| | CGCCGAGTTGAAAGAGATCGAAACCCAAAAGACTCTTCAGAAAATTAACGAGTCTCGTAGTTGGTTC |
| | TTCGAGCGGATTAATAAGATAGACAGACCTCTGGCACGACTGATTAAGAAGAAGCGCGAAAAGAACC |
| | AGATTGATACCATCAAGAACGACAAGGGCGACATCACTACTGACCCGACCGAGATCCAGACCACTAT |
| | TCGGGAGTATTATAAGCATTTGTATGCTAACAAGCTTGAGAACCTGGAAGAGATGGACACTTTTCTG |
| | GATACCTATACTCTGCCACGGCTTAATCAAGAGGAAGTCGAGTCCCTCAACCGCCCAATTACAGGAA |
| | GCGAGATTGTGGCCATAATTAACTCCCTGCCGACAAAGAAATCTCCTGGTCCGGACGGGTTTACAGC |
| | TGAGTTTTATCAACGGTATATGGAAGAGCTTGTACCGTTTCTGCTCAAGCTCTTTCAGTCTATAGAA |
| | AAGGAAGGCATCTTGCCCAATTCCTTCTACGAAGCTTCTATAATACTTATTCCCAAACCAGGACGCG |
| | ATACCACAAAGAAGGAAAACTTCCGGCCCATTAGTCTCATGAATATCGACGCTAAAATATTGAACAA |
| | GATTCTCGCCAACAGAATCCAACAACATATTAAGAAATTGATACATCACGACCAGGTGGGGTTTATA |
| | CCTGGCATGCAGGGCTGGTTTAACATCCGGAAGAGTATTAACGTCATTCAACACATTAATAGAGCTA |
| | AGGATAAGAATCATATGATCATCTCTATAGACGCGGAAAAGGCATTCGATAAGATTCAGCAGCCATT |
| | TATGCTCAAGACTCTGAACAAACTCGGCATCGACGGAACATATTTTAAGATTATTCGCGCAATTTAC |
| | GATAAGCCGACTGCTAACATTATCCTTAACGGCCAAAAGCTCGAGGCCTTTCCGCTCAAGACTGGAA |
| | CCCGCCAAGGCTGTCCCCTCTCCCCGCTTTTGTTTAATATTGTACTCGAGGTGCTGGCTAGGGCTAT |
| | TCGTCAAGAGAAAGAGATTAAAGGGATACAGCTCGGGAAGGAAGAGGTCAAGCTTTCCTTGTTCGCC |
| | GATGATATGATTGTGTACCTGGAGAATCCTATTGTGTCTGCTCAGAACCTTCTTAAACTTATTTCTA |
| | ACTTTAGCAAGGTCAGCGGCTATAAGATTAACGTCCAGAAATCTCAGGCCTTTCTGTACACAAATAA |
| | TCGACAGACCGAATCCCAGATAATGGGTGAGCTTCCGTTTGTCATAGCCAGCAAAAGGATAAAGTAT |
| | CTCGGAATCCAGCTGACACGAGACGTTAAAGATTTGTTTAAGGAAAATTACAAGCCTCTCCTGAAAG |
| | AGATTAAGGAAGATACTAATAAGTGGAAGAATATCCCCTGTTCATGGGTTGGCAGAATCAACATAGT |
| | GAAGATGGCAATACTTCCTAAAGTGATATATCGCTTTAACGCCATCCCAATTAAACTGCCTATGACC |
| | TTCTTTACGGAGCTCGAGAAAACAACCCTTAAATTTATATGGAATCAAAAGAGAGCAAGAATAGCGA |
| | AGTCCATCTTGAGCCAGAAGAATAAGGCCGGTGGGATTACTTTGCCTGATTTTAAGTTGTATTATAA |
| | AGCCACAGTAACTAAGACAGCCTGGTATTGGTATCAGAATAGAGACATCGACCAGTGGAATCGGACC |
| | GAACCATCAGAGATAATGCCCCACATCTATAATTACCTTATATTCGATAAGCCAGAAAAGAATAAAC |
| | AGTGGGGCAAAGACAGCCTCTTCAACAAGTGGTGTTGGGAGAATTGGCTGGCCATATGCCGGAAACT |
| | CAAGCTCGACCCCTTTCTTACACCCTACACTAAAATCAACAGTAGGTGGATCAAGGACTTGAATGTC |
| | AAGCCAAAGACTATAAAGACACTGGAAGAGAATCTTGGGATCACAATACAAGATATAGGCGTCGGCA |
| | AAGATTTTATGTCAAAGACGCCCAAGGCCATGGCCACTAAGGATAAGATTGATAAGTGGGACCTTAT |
| | TAAGCTCAAAAGCTTCTGTACTGCCAAGGAGACCACGATCAGAGTTAATAGGCAGCCCACTACATGG |
| | GAAAAGATTTTCGCCACTTATTCATCAGATAAGGGGTTGATAAGCAGAATATATAACGAGCTGAAGC |
| | AGATCTACAAGAAGAAAACGAATAATCCCATCAAGAAGTGGGCAAAAGATATGAACAGGCATTTTAG |
| | CAAAGAGGATATCTACGCCGCGAAGAAGCATATGAAGAAGTGTAGTTCAAGCTTGGCCATTCGTGAG |
| | ATGCAGATTAAGACGACCATGCGATACCACCTTACCCCAGTGAGGATGGCAATTATCAAGAAATCTG |
| | GCAATAATAGATGTTGGCGGGGCTGTGGCGAGATTGGCACCCTGCTCCATTGCTGGTGGGATTGCAA |
| | GCTGGTGCAGCCGCTTTGGAAATCAGTCTGGCGCTTTCTGAGGGACCTCGAGCTTGAGATTCCCTTC |
| | GATCCCGCAATTCCCTTGCTCGGAATCTATCCTAACGAATACAAGAGCTGTTGTTACAAGGATACGT |
| | GTACCCGGATGTTCATCGCGGCCTTGTTTACGATAGCTAAGACGTGGAATCAGCCTAAGTGCCCCAC |
| | AATGATCGATTGGATCAAGAAAATGTGGCATATTTATACCATGGAGTATTACGCAGCAATTAAGAAT |
| | GACGAATTTATTTCCTTCGTTGGGACCTGGATGAAGCTGGAGACTATTATTCTGAGCAAGCTGTCTC |
| | AGGAGCAAAAGACAAAGCATAGAATCTTCTCTCTCATTGGTGGTAACGACTACAAAGACGATGACGA |
| | CAAGTAAAGCGCTTCTAGAAGTTGTCTCCTCCTGCACTGACTGACTGATACAATCGATTTCTGGATC |
| | CGCAGGCCTAATCAACCTCTGGATTACAAAATTTGTGAAAGATTGACTGGTATTCTTAACTATGTTG |
| | CTCCTTTTACGCTATGTGGATACGCTGCTTTAATGCCTTTGTATCATGCTATTGCTTCCCGTATGGC |
| | TTTCATTTTCTCCTCCTTGTATAAATCCTGGTTGCTGTCTCTTTATGAGGAGTTGTGGCCCGTTGTC |
| | AGGCAACGTGGCGTGGTGTGCACTGTGTTTGCTGACGCAACCCCCACTGGTTGGGGCATTGCCACCA |
| | CCTGTCAGCTCCTTTCCGGGACTTTCGCTTTCCCCCTCCCTATTGCCACGGCGGAACTCATCGCCGC |
| | CTGCCTTGCCCGCTGCTGGACAGGGGCTCGGCTGTTGGGCACTGACAATTCCGTGGTGTTGTCGGGG |
| | AAGCTGACGTCCTTTCCATGGCTGCTCGCCTGTGTTGCCACCTGGATTCTGCGCGGGACGTCCTTCT |
| | GCTACGTCCCTTCGGCCCTCAATCCAGCGGACCTTCCTTCCCGCtgagagacacaaaaaattccaac |
| | acactattgcaatgaaaataaatttcctttattagccagaagtcagatgctcaaggggcttcatgat |
| | gtccccataatttttggcagagggaaaaagatctcagtggtatttgtgagccagggcattggccttc |
| | tgataggcagcctgcacctgaggagtgcggccgctttacttgtacagctcgtccatgccgagagtga |
| | tcccggcggcggtcacgaactccagcaggaccatgtgatcgcgcttctcgttggggtctttgctcag |
| | ggcggactgggtgctcaggtagtggttgtcgggcagcagcacggggccgtcgccgatgggggtgttc |
| | tgctggtagtggtcggcgagctgcacgctgccgtcctcgatgttgtggcggatcttgaagttcacct |
| | tgatgccgttcttctgcttgtcggccatgatatagacgttgtggctgttgtagttgtactccagctt |
| | gtgccccaggatgttgccgtcctccttgaagtcgatgcccttcagctcgatgcggttcaccagggtg |
| | tcgccctcgaacttcacctcggcgcgggtcttgtagttgccgtcgtccttgaagaagatggtgcgct |
| | cctggacgtagccttcgggcatggcggacttgaagaagtcgtgctgcttcatgtggtcggggtagcg |
| | gctgaagcactgcacgccgtaggtcagggtggtcacgagggtgggccagggcacgggcagcttgccg |
| | gtggtgcagatgaacttcagggtcagcttgccgtaggtggcatcgccctcgccctcgccggacacgc |
| | tgaacttgtggccgtttacgtcgccgtccagctcgaccaggatgggcaccaccccggtgaacagctc |
| | ctcgcccttgctcaccatggtggcgggatctgacggttcactaaaccagctctgcttatatagacct |
| | cccaccgtacacgcctaccgcccatttgcgtcaatggggcggagttgttacgacattttggaaagtc |
| | ccgttgattttggtgccaaaacaaactcccattgacgtcaatggggtggagacttggaaatccccgt |
| | gagtcaaaccgctatccacgcccattgatgtactgccaaaaccgcatcaccatggtaatagcgatga |
| | ctaatacgtagatgtactgccaagtaggaaagtcccataaggtcatgtactgggcataatgccaggc |
| | gggccatttaccgtcattgacgtcaatagggggcgtacttggcatatgatacacttgatgtactgcc |
| | aagtgggcagtttaccgtaaatactccacccattgacgtcaatggaaagtccctattggcgttacta |
| | tgggaacatacgtcattattgacgtcaatgggcgggggtcgttgggcggtcagccaggcgggccatt |
| | taccgtaagttatgtaacgGGCCTGCTGCCGGCTCTGCGGCCTCTTCCGCGTCTTCGCCTTCGCCCT |
| | CAGACGAGTCGGATCTCCCTTTGGGCCGCCTCCCCGCCTGTCTAGCTTGACTGACTGAGATACAGCG |
| | TACCTTCAGCTCACAGACATGATAAGATACATTGATGAGTTTGGACAAACCACAACTAGAATGCAGT |
| | GAAAAAAATGCTTTATTTGTGAAATTTGTGATGCTATTGCTTTATTTGTAACCATTATAAGCTGCAA |
| | TAAACAAGTTAACAACAACAATTGCATTCATTTTATGTTTCAGGTTCAGGGGGAGGTGTGGGAGGTT |
| | TTTTAAAGCAAGTAAAACCTCTACAAATGTGGTATTGGCCCATCTCTATCGGTATCGTAGCATAACC |
| | CCTTGGGGCCTCTAAACGGGTCTTGAGGGGTTTTTTGTGCCCCTCGGGCCGGATTGCTATCTACCGG |
| | CATTGGCGCAGAAAAAAATGCCTGATGCGACGCTGCGCGTCTTATACTCCCACATATGCCAGATTCA |
| | GCAACGGATACGGCTTCCCCAACTTGCCCACTTCCATACGTGTCCTCCTTACCAGAAATTTATCCTT |
| | AAGGTCGTCAGCTATCCTGCAGGCGATCTCTCGATTTCGATCAAGACATTCCTTTAATGGTCTTTTC |
| | TGGACACCACTAGGGGTCAGAAGTAGTTCATCAAACTTTCTTCCCTCCCTAATCTCATTGGTTACCT |
| | TGGGCTATCGAAACTTAATTAAGCGATCTGCATCTCAATTAGTCAGCAACCATAGTCCCGCCCCTAA |
| | CTCCGCCCATCCCGCCCCTAACTCCGCCCAGTTCCGCCCATTCTCCGCCCCATCGCTGACTAATTTT |
| | TTTTATTTATGCAGAGGCCGAGGCCGCCTCGGCCTCTGAGCTATTCCAGAAGTAGTGAGGAGGCTTT |
| | TTTGGAGGCCTAGGCTTTTGCAAAGGAGGTAGCCAACATGATTGAACAAGATGGATTGCACGCAGGT |
| | TCTCCCGCCGCTTGGGTGGAGAGGCTATTCGGCTATGACTGGGCACAACAGACAATCGGCTGCTCTG |
| | ATGCCGCCGTGTTCCGGCTGTCAGCGCAGGGGCGCCCGGTTCTTTTTGTCAAGACCGACCTGTCCGG |
| | TGCCCTGAATGAACTCCAGGACGAGGCAGCGCGGCTATCGTGGCTGGCCACGACGGGCGTTCCTTGC |
| | GCAGCTGTGCTCGACGTTGTCACTGAAGCGGGAAGGGACTGGCTGCTATTGGGCGAAGTGCCGGGGC |
| | AGGATCTCCTGTCATCTCACCTTGCTCCTGCCGAGAAAGTATCCATCATGGCTGATGCAATGCGGCG |
| | GCTGCATACGCTTGATCCGGCTACCTGCCCATTCGACCACCAAGCGAAACATCGCATCGAGCGAGCA |
| | CGTACTCGGATGGAAGCCGGTCTTGTCGATCAGGATGATCTGGACGAAGAGCATCAGGGGCTCGCGC |
| | CAGCCGAACTGTTCGCCAGGCTCAAGGCGCGGATGCCCGACGGCGAGGATCTCGTCGTGACCCACGG |
| | CGATGCCTGCTTGCCGAATATCATGGTGGAAAATGGCCGCTTTTCTGGATTCATCGACTGTGGCCGG |
| | CTGGGTGTGGCGGACCGCTATCAGGACATAGCGTTGGCTACCCGTGATATTGCTGAAGAGCTTGGCG |
| | GCGAATGGGCTGACCGCTTCCTCGTGCTTTACGGTATCGCCGCTCCCGATTCGCAGCGCATCGCCTT |
| | CTATCGCCTTCTTGACGAGTTCTTCTAGTATGTAAGCCCTGTGCCTTCTAGTTGCCAGCCATCTGTT |
| | GTTTGCCCCTCCCCCGTGCCTTCCTTGACCCTGGAAGGTGCCACTCCCACTGTCCTTTCCTAATAAA |
| | ATGAGGAAATTGCATCGCATTGTCTGAGTAGGTGTCATTCTATTCTGGGGGGTGGGGTGGGGCAGGA |
| | CAGCAAGGGGGAGGATTGGGAAGACAATAGCAGGCATGCTGGGGATGCGGTGGGCTCTATGGTTAAT |
| | TAACCAGTCAAGTCAGCTACTTGGCGAGATCGACTTGTCTGGGTTTCGACTACGCTCAGAATTGCGT |
| | CAGTCAAGTTCGATCTGGTCCTTGCTATTGCACCCGTTCTCCGATTACGAGTTTCATTTAAATCATG |
| | TGAGCAAAAGGCCAGCAAAAGGCCAGGAACCGTAAAAAGGCCGCGTTGCTGGCGTTTTTCCATAGGC |
| | TCCGCCCCCCTGACGAGCATCACAAAAATCGACGCTCAAGTCAGAGGTGGCGAAACCCGACAGGACT |
| | ATAAAGATACCAGGCGTTTCCCCCTGGAAGCTCCCTCGTGCGCTCTCCTGTTCCGACCCTGCCGCTT |
| | ACCGGATACCTGTCCGCCTTTCTCCCTTCGGGAAGCGTGGCGCTTTCTCATAGCTCACGCTGTAGGT |
| | ATCTCAGTTCGGTGTAGGTCGTTCGCTCCAAGCTGGGCTGTGTGCACGAACCCCCCGTTCAGCCCGA |
| | CCGCTGCGCCTTATCCGGTAACTATCGTCTTGAGTCCAACCCGGTAAGACACGACTTATCGCCACTG |
| | GCAGCAGCCACTGGTAACAGGATTAGCAGAGCGAGGTATGTAGGCGGTGCTACAGAGTTCTTGAAGT |
| | GGTGGCCTAACTACGGCTACACTAGAAGAACAGTATTTGGTATCTGCGCTCTGCTGAAGCCAGTTAC |
| | CTTCGGAAAAAGAGTTGGTAGCTCTTGATCCGGCAAACAAACCACCGCTGGTAGCGGTGGTTTTTTT |
| | GTTTGCAAGCAGCAGATTACGCGCAGAAAAAAAGGATCTCAAGAAGATCCTTTGATCTTTTCTACGG |
| | GGTCTGACGCTCAGTGGAACGAAAACTCACGTTAAGGGATTTTGGTCATGAGATTATCAAAAAGGAT |
| | CTTCACCTAGATCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCTATTTCGTTCAT |
| | CCATAGTTGCATTTAAATTTCCGAACTCTCCAAGGCCCTCGTCGGAAAATCTTCAAACCTTTCGTCC |
| | GATCCATCTTGCAGGCTACCTCTCGAACGAACTATCGCAAGTCTCTTGGCCGGCCTTGCGCCTTGGC |
| | TATTGCTTGGCAGCGCCTATCGCCAGGTATTACTCCAATCCCGAATATCCGAGATCGGGATCACCCG |
| | AGAGAAGTTCAACCTACATCCTCAATCCCGATCTATCCGAGATCCGAGGAATATCGAAATCGGGGCG |
| | CGCCTGGTGTACCGAGAACGATCCTCTCAGTGCGAGTCTC (SEQ ID NO: 102) |
| RET- | AGATCCGAGGAATATCGAAATCGGGGCGCGCCTGGTGTACCGAGAACGATCCTCTCAGTGCGAGTCT |
| 013 | CGACGATCCATATCGTTGCTTGGCAGTCAGCCAGTCGGAATCCAGCTTGGGACCCAGGAAGTCCAAT |
| LINE1- | CGTCAGATATTGTACTCAAGCCTGGTCACGGCAGCGTACCGATCTGTTTAAACCTAGATATTGATAG |
| GFP | TCTGATCGGTCAACGTATAATCGAGTCCTAGCTTTTGCAAACATCTATCAAGAGACAGGATCAGCAG |
| ORF2-N | GAGGCTTTCGCATGAGTATTCAACATTTCCGTGTCGCCCTTATTCCCTTTTTTGCGGCATTTTGCCT |
| Nucleo- | TCCTGTTTTTGCTCACCCAGAAACGCTGGTGAAAGTAAAAGATGCTGAAGATCAGTTGGGTGCGCGA |
| plasmin | GTGGGTTACATCGAACTGGATCTCAACAGCGGTAAGATCCTTGAGAGTTTTCGCCCCGAAGAACGCT |
| NLS | TTCCAATGATGAGCACTTTTAAAGTTCTGCTATGTGGCGCGGTATTATCCCGTATTGACGCCGGGCA |
| Linker | AGAGCAACTCGGTCGCCGCATACACTATTCTCAGAATGACTTGGTTGAGTATTCACCAGTCACAGAA |
| | AAGCATCTTACGGATGGCATGACAGTAAGAGAATTATGCAGTGCTGCCATAACCATGAGTGATAACA |
| | CTGCGGCCAACTTACTTCTGACAACGATTGGAGGACCGAAGGAGCTAACCGCTTTTTTGCACAACAT |
| | GGGGGATCATGTAACTCGCCTTGATCGTTGGGAACCGGAGCTGAATGAAGCCATACCAAACGACGAG |
| | CGTGACACCACGATGCCTGTAGCAATGGCAACAACCTTGCGTAAACTATTAACTGGCGAACTACTTA |
| | CTCTAGCTTCCCGGCAACAGTTGATAGACTGGATGGAGGCGGATAAAGTTGCAGGACCACTTCTGCG |
| | CTCGGCCCTTCCGGCTGGCTGGTTTATTGCTGATAAATCTGGAGCCGGTGAGCGTGGGTCTCGCGGT |
| | ATCATTGCAGCACTGGGGCCAGATGGTAAGCCCTCCCGTATCGTAGTTATCTACACGACGGGGAGTC |
| | AGGCAACTATGGATGAACGAAATAGACAGATCGCTGAGATAGGTGCCTCACTGATTAAGCATTGGTA |
| | ACCGATTCTAGGTGCATTGGCGCAGAAAAAAATGCCTGATGCGACGCTGCGCGTCTTATACTCCCAC |
| | ATATGCCAGATTCAGCAACGGATACGGCTTCCCCAACTTGCCCACTTCCATACGTGTCCTCCTTACC |
| | AGAAATTTATCCTTAAGATCGTTTAAACTCGACTCTGGCTCTATCGAATCTCCGTCGTTTCGAGCTT |
| | ACGCGAACAGCCGTGGCGCTCATTTGCTCGTCGGGCATCGAATCTCGTCAGCTATCGTCAGCTTACC |
| | TTTTTGGCAGCGATCGCGGCTCCCGACATCTTGGACCATTAGCTCCACAGGTATCTTCTTCCCTCTA |
| | GTGGTCATAACAGCAGCTTCAGCTACCTCTCAATTCAAAAAACCCCTCAAGACCCGTTTAGAGGCCC |
| | CAAGGGGTTATGCTATCAATCGTTGCGTTACACACACAAAAAACCAACACACATCCATCTTCGATGG |
| | ATAGCGATTTTATTATCTAACTGCTGATCGAGTGTAGCCAGATCTAGTAATCAATTACGGGGTCATT |
| | AGTTCATAGCCCATATATGGAGTTCCGCGTTACATAACTTACGGTAAATGGCCCGCCTGGCTGACCG |
| | CCCAACGACCCCCGCCCATTGACGTCAATAATGACGTATGTTCCCATAGTAACGCCAATAGGGACTT |
| | TCCATTGACGTCAATGGGTGGAGTATTTACGGTAAACTGCCCACTTGGCAGTACATCAAGTGTATCA |
| | TATGCCAAGTACGCCCCCTATTGACGTCAATGACGGTAAATGGCCCGCCTGGCATTATGCCCAGTAC |
| | ATGACCTTATGGGACTTTCCTACTTGGCAGTACATCTACGTATTAGTCATCGCTATTACCATGCTGA |
| | TGCGGTTTTGGCAGTACATCAATGGGCGTGGATAGCGGTTTGACTCACGGGGATTTCCAAGTCTCCA |
| | CCCCATTGACGTCAATGGGAGTTTGTTTTGGCACCAAAATCAACGGGACTTTCCAAAATGTCGTAAC |
| | AACTCCGCCCCATTGACGCAAATGGGCGGTAGGCGTGTACGGTGGGAGGTCTATATAAGCAGAGCTG |
| | GTTTAGTGAACCGTCAGATCAGATCTTTGTCGATCCTACCATCCACTCGACACACCCGCCAGCGGCC |
| | GCTAATACGACTCACTATAGGGAGAAGTACTGCCACCATGGGCAAGAAGCAAAATCGCAAGACGGGG |
| | AATTCCAAGACACAATCCGCTAGCCCACCACCTAAAGAGCGTTCTAGCTCCCCTGCTACTGAGCAGT |
| | CCTGGATGGAAAACGACTTCGATGAACTCCGGGAAGAGGGATTTAGGCGATCCAACTATTCAGAACT |
| | CCGCGAAGATATCCAGACAAAGGGGAAGGAAGTCGAGAATTTCGAGAAGAACCTCGAGGAGTGCATC |
| | ACCCGTATCACAAACACTGAGAAATGTCTCAAAGAACTCATGGAACTTAAGACAAAAGCCAGGGAGC |
| | TTCGAGAGGAGTGTCGGAGTCTGAGATCCAGGTGTGACCAGCTCGAGGAGCGCGTGAGCGCGATGGA |
| | AGACGAGATGAACGAGATGAAAAGAGAGGGCAAATTCAGGGAGAAGCGCATTAAGAGGAACGAACAG |
| | AGTCTGCAGGAGATTTGGGATTACGTCAAGAGGCCTAACCTGCGGTTGATCGGCGTCCCCGAGAGCG |
| | ACGTAGAAAACGGGACTAAACTGGAGAATACACTTCAAGACATCATTCAAGAAAATTTTCCAAACCT |
| | GGCTCGGCAAGCTAATGTGCAAATCCAAGAGATCCAACGCACACCCCAGCGGTATAGCTCTCGGCGT |
| | GCCACCCCTAGGCATATTATCGTGCGCTTTACTAAGGTGGAGATGAAAGAGAAGATGCTGCGAGCCG |
| | CTCGGGAAAAGGGAAGGGTGACTTTGAAGGGCAAACCTATTCGGCTGACGGTTGACCTTAGCGCCGA |
| | GACACTCCAGGCACGCCGGGAATGGGGCCCCATCTTTAATATCCTGAAGGAGAAGAACTTCCAGCCA |
| | CGAATCTCTTACCCTGCAAAGTTGAGTTTTATCTCCGAGGGTGAGATTAAGTATTTCATCGATAAAC |
| | AGATGCTGCGAGACTTCGTGACAACTCGCCCAGCTCTCAAGGAACTGCTCAAAGAGGCTCTTAATAT |
| | GGAGCGCAATAATAGATATCAACCCTTGCAGAACCACGCAAAGATGTGAGACAGCCGTCAGACCATC |
| | AAGACTAGGAAGAAACTGCATCAACTAATGAGCAAAATCACCAGCTAACATCATAGTATACATGaaa |
| | aggccggcggccacgaaaaaggccggccaggcaaaaaagaaaaagggcggcggcagcACCGGCTCTA |
| | ACTCACATATCACCATCCTTACACTTAACATTAACGGCCTCAACTCAGCTATCAAGCGCCATCGGCT |
| | GGCCAGCTGGATCAAATCACAGGATCCAAGCGTTTGTTGCATCCAAGAGACCCACCTGACCTGTAGA |
| | GATACTCACCGCCTCAAGATCAAGGGATGGCGAAAGATTTATCAGGCGAACGGTAAGCAGAAGAAAG |
| | CCGGAGTCGCAATTCTGGTCTCAGACAAGACGGATTTCAAGCCCACCAAAATTAAGCGTGATAAGGA |
| | AGGTCACTATATTATGGTGAAAGGCAGCATACAGCAGGAAGAACTTACCATATTGAACATCTACGCG |
| | CCAAACACCGGCGCACCTCGCTTTATCAAACAGGTCCTGTCCGATCTGCAGCGAGATCTGGATTCTC |
| | ATACGTTGATTATGGGTGATTTCAATACACCATTGAGCACCCTGGATCGCAGCACCAGGCAAAAGGT |
| | AAATAAAGACACGCAAGAGCTCAATAGCGCACTGCATCAGGCAGATCTCATTGATATTTATCGCACT |
| | CTTCATCCTAAGAGTACCGAGTACACATTCTTCAGCGCCCCACATCATACATACTCAAAGATCGATC |
| | ATATCGTCGGCTCAAAGGCTCTGCTGTCAAAGTGCAAGCGCACAGAGATAATTACAAATTACCTGTC |
| | AGATCATAGCGCGATCAAGCTCGAGCTGAGAATCAAGAACCTGACCCAGAGCCGGAGTACCACTTGG |
| | AAGCTTAATAACCTGCTGCTCAACGATTATTGGGTCCACAATGAGATGAAGGCAGAGATTAAAATGT |
| | TCTTCGAAACAAATGAGAATAAGGATACTACCTATCAAAACCTTTGGGATGCCTTTAAGGCCGTCTG |
| | CAGAGGCAAGTTCATCGCCCTCAACGCCTATAAAAGAAAACAAGAGAGATCTAAGATCGATACTCTC |
| | ACCTCTCAGCTGAAGGAGTTGGAGAAACAGGAACAGACCCACTCCAAGGCGTCAAGACGGCAGGAGA |
| | TCACAAAGATTCGCGCCGAGTTGAAAGAGATCGAAACCCAAAAGACTCTTCAGAAAATTAACGAGTC |
| | TCGTAGTTGGTTCTTCGAGCGGATTAATAAGATAGACAGACCTCTGGCACGACTGATTAAGAAGAAG |
| | CGCGAAAAGAACCAGATTGATACCATCAAGAACGACAAGGGCGACATCACTACTGACCCGACCGAGA |
| | TCCAGACCACTATTCGGGAGTATTATAAGCATTTGTATGCTAACAAGCTTGAGAACCTGGAAGAGAT |
| | GGACACTTTTCTGGATACCTATACTCTGCCACGGCTTAATCAAGAGGAAGTCGAGTCCCTCAACCGC |
| | CCAATTACAGGAAGCGAGATTGTGGCCATAATTAACTCCCTGCCGACAAAGAAATCTCCTGGTCCGG |
| | ACGGGTTTACAGCTGAGTTTTATCAACGGTATATGGAAGAGCTTGTACCGTTTCTGCTCAAGCTCTT |
| | TCAGTCTATAGAAAAGGAAGGCATCTTGCCCAATTCCTTCTACGAAGCTTCTATAATACTTATTCCC |
| | AAACCAGGACGCGATACCACAAAGAAGGAAAACTTCCGGCCCATTAGTCTCATGAATATCGACGCTA |
| | AAATATTGAACAAGATTCTCGCCAACAGAATCCAACAACATATTAAGAAATTGATACATCACGACCA |
| | GGTGGGGTTTATACCTGGCATGCAGGGCTGGTTTAACATCCGGAAGAGTATTAACGTCATTCAACAC |
| | ATTAATAGAGCTAAGGATAAGAATCATATGATCATCTCTATAGACGCGGAAAAGGCATTCGATAAGA |
| | TTCAGCAGCCATTTATGCTCAAGACTCTGAACAAACTCGGCATCGACGGAACATATTTTAAGATTAT |
| | TCGCGCAATTTACGATAAGCCGACTGCTAACATTATCCTTAACGGCCAAAAGCTCGAGGCCTTTCCG |
| | CTCAAGACTGGAACCCGCCAAGGCTGTCCCCTCTCCCCGCTTTTGTTTAATATTGTACTCGAGGTGC |
| | TGGCTAGGGCTATTCGTCAAGAGAAAGAGATTAAAGGGATACAGCTCGGGAAGGAAGAGGTCAAGCT |
| | TTCCTTGTTCGCCGATGATATGATTGTGTACCTGGAGAATCCTATTGTGTCTGCTCAGAACCTTCTT |
| | AAACTTATTTCTAACTTTAGCAAGGTCAGCGGCTATAAGATTAACGTCCAGAAATCTCAGGCCTTTC |
| | TGTACACAAATAATCGACAGACCGAATCCCAGATAATGGGTGAGCTTCCGTTTGTCATAGCCAGCAA |
| | AAGGATAAAGTATCTCGGAATCCAGCTGACACGAGACGTTAAAGATTTGTTTAAGGAAAATTACAAG |
| | CCTCTCCTGAAAGAGATTAAGGAAGATACTAATAAGTGGAAGAATATCCCCTGTTCATGGGTTGGCA |
| | GAATCAACATAGTGAAGATGGCAATACTTCCTAAAGTGATATATCGCTTTAACGCCATCCCAATTAA |
| | ACTGCCTATGACCTTCTTTACGGAGCTCGAGAAAACAACCCTTAAATTTATATGGAATCAAAAGAGA |
| | GCAAGAATAGCGAAGTCCATCTTGAGCCAGAAGAATAAGGCCGGTGGGATTACTTTGCCTGATTTTA |
| | AGTTGTATTATAAAGCCACAGTAACTAAGACAGCCTGGTATTGGTATCAGAATAGAGACATCGACCA |
| | GTGGAATCGGACCGAACCATCAGAGATAATGCCCCACATCTATAATTACCTTATATTCGATAAGCCA |
| | GAAAAGAATAAACAGTGGGGCAAAGACAGCCTCTTCAACAAGTGGTGTTGGGAGAATTGGCTGGCCA |
| | TATGCCGGAAACTCAAGCTCGACCCCTTTCTTACACCCTACACTAAAATCAACAGTAGGTGGATCAA |
| | GGACTTGAATGTCAAGCCAAAGACTATAAAGACACTGGAAGAGAATCTTGGGATCACAATACAAGAT |
| | ATAGGCGTCGGCAAAGATTTTATGTCAAAGACGCCCAAGGCCATGGCCACTAAGGATAAGATTGATA |
| | AGTGGGACCTTATTAAGCTCAAAAGCTTCTGTACTGCCAAGGAGACCACGATCAGAGTTAATAGGCA |
| | GCCCACTACATGGGAAAAGATTTTCGCCACTTATTCATCAGATAAGGGGTTGATAAGCAGAATATAT |
| | AACGAGCTGAAGCAGATCTACAAGAAGAAAACGAATAATCCCATCAAGAAGTGGGCAAAAGATATGA |
| | ACAGGCATTTTAGCAAAGAGGATATCTACGCCGCGAAGAAGCATATGAAGAAGTGTAGTTCAAGCTT |
| | GGCCATTCGTGAGATGCAGATTAAGACGACCATGCGATACCACCTTACCCCAGTGAGGATGGCAATT |
| | ATCAAGAAATCTGGCAATAATAGATGTTGGCGGGGCTGTGGCGAGATTGGCACCCTGCTCCATTGCT |
| | GGTGGGATTGCAAGCTGGTGCAGCCGCTTTGGAAATCAGTCTGGCGCTTTCTGAGGGACCTCGAGCT |
| | TGAGATTCCCTTCGATCCCGCAATTCCCTTGCTCGGAATCTATCCTAACGAATACAAGAGCTGTTGT |
| | TACAAGGATACGTGTACCCGGATGTTCATCGCGGCCTTGTTTACGATAGCTAAGACGTGGAATCAGC |
| | CTAAGTGCCCCACAATGATCGATTGGATCAAGAAAATGTGGCATATTTATACCATGGAGTATTACGC |
| | AGCAATTAAGAATGACGAATTTATTTCCTTCGTTGGGACCTGGATGAAGCTGGAGACTATTATTCTG |
| | AGCAAGCTGTCTCAGGAGCAAAAGACAAAGCATAGAATCTTCTCTCTCATTGGTGGTAACGACTACA |
| | AAGACGATGACGACAAGTAAAGCGCTTCTAGAAGTTGTCTCCTCCTGCACTGACTGACTGATACAAT |
| | CGATTTCTGGATCCGCAGGCCTAATCAACCTCTGGATTACAAAATTTGTGAAAGATTGACTGGTATT |
| | CTTAACTATGTTGCTCCTTTTACGCTATGTGGATACGCTGCTTTAATGCCTTTGTATCATGCTATTG |
| | CTTCCCGTATGGCTTTCATTTTCTCCTCCTTGTATAAATCCTGGTTGCTGTCTCTTTATGAGGAGTT |
| | GTGGCCCGTTGTCAGGCAACGTGGCGTGGTGTGCACTGTGTTTGCTGACGCAACCCCCACTGGTTGG |
| | GGCATTGCCACCACCTGTCAGCTCCTTTCCGGGACTTTCGCTTTCCCCCTCCCTATTGCCACGGCGG |
| | AACTCATCGCCGCCTGCCTTGCCCGCTGCTGGACAGGGGCTCGGCTGTTGGGCACTGACAATTCCGT |
| | GGTGTTGTCGGGGAAGCTGACGTCCTTTCCATGGCTGCTCGCCTGTGTTGCCACCTGGATTCTGCGC |
| | GGGACGTCCTTCTGCTACGTCCCTTCGGCCCTCAATCCAGCGGACCTTCCTTCCCGCtgagagacac |
| | aaaaaattccaacacactattgcaatgaaaataaatttcctttattagccagaagtcagatgctcaa |
| | ggggcttcatgatgtccccataatttttggcagagggaaaaagatctcagtggtatttgtgagccag |
| | ggcattggccttctgataggcagcctgcacctgaggagtgcggccgctttacttgtacagctcgtcc |
| | atgccgagagtgatcccggcggcggtcacgaactccagcaggaccatgtgatcgcgcttctcgttgg |
| | ggtctttgctcagggcggactgggtgctcaggtagtggttgtcgggcagcagcacggggccgtcgcc |
| | gatgggggtgttctgctggtagtggtcggcgagctgcacgctgccgtcctcgatgttgtggcggatc |
| | ttgaagttcaccttgatgccgttcttctgcttgtcggccatgatatagacgttgtggctgttgtagt |
| | tgtactccagcttgtgccccaggatgttgccgtcctccttgaagtcgatgcccttcagctcgatgcg |
| | gttcaccagggtgtcgccctcgaacttcacctcggcgcgggtcttgtagttgccgtcgtccttgaag |
| | aagatggtgcgctcctggacgtagccttcgggcatggcggacttgaagaagtcgtgctgcttcatgt |
| | ggtcggggtagcggctgaagcactgcacgccgtaggtcagggtggtcacgagggtgggccagggcac |
| | gggcagcttgccggtggtgcagatgaacttcagggtcagcttgccgtaggtggcatcgccctcgccc |
| | tcgccggacacgctgaacttgtggccgtttacgtcgccgtccagctcgaccaggatgggcaccaccc |
| | cggtgaacagctcctcgcccttgctcaccatggtggcgggatctgacggttcactaaaccagctctg |
| | cttatatagacctcccaccgtacacgcctaccgcccatttgcgtcaatggggcggagttgttacgac |
| | attttggaaagtcccgttgattttggtgccaaaacaaactcccattgacgtcaatggggtggagact |
| | tggaaatccccgtgagtcaaaccgctatccacgcccattgatgtactgccaaaaccgcatcaccatg |
| | gtaatagcgatgactaatacgtagatgtactgccaagtaggaaagtcccataaggtcatgtactggg |
| | cataatgccaggcgggccatttaccgtcattgacgtcaatagggggcgtacttggcatatgatacac |
| | ttgatgtactgccaagtgggcagtttaccgtaaatactccacccattgacgtcaatggaaagtccct |
| | attggcgttactatgggaacatacgtcattattgacgtcaatgggcgggggtcgttgggcggtcagc |
| | caggcgggccatttaccgtaagttatgtaacgGGCCTGCTGCCGGCTCTGCGGCCTCTTCCGCGTCT |
| | TCGCCTTCGCCCTCAGACGAGTCGGATCTCCCTTTGGGCCGCCTCCCCGCCTGTCTAGCTTGACTGA |
| | CTGAGATACAGCGTACCTTCAGCTCACAGACATGATAAGATACATTGATGAGTTTGGACAAACCACA |
| | ACTAGAATGCAGTGAAAAAAATGCTTTATTTGTGAAATTTGTGATGCTATTGCTTTATTTGTAACCA |
| | TTATAAGCTGCAATAAACAAGTTAACAACAACAATTGCATTCATTTTATGTTTCAGGTTCAGGGGGA |
| | GGTGTGGGAGGTTTTTTAAAGCAAGTAAAACCTCTACAAATGTGGTATTGGCCCATCTCTATCGGTA |
| | TCGTAGCATAACCCCTTGGGGCCTCTAAACGGGTCTTGAGGGGTTTTTTGTGCCCCTCGGGCCGGAT |
| | TGCTATCTACCGGCATTGGCGCAGAAAAAAATGCCTGATGCGACGCTGCGCGTCTTATACTCCCACA |
| | TATGCCAGATTCAGCAACGGATACGGCTTCCCCAACTTGCCCACTTCCATACGTGTCCTCCTTACCA |
| | GAAATTTATCCTTAAGGTCGTCAGCTATCCTGCAGGCGATCTCTCGATTTCGATCAAGACATTCCTT |
| | TAATGGTCTTTTCTGGACACCACTAGGGGTCAGAAGTAGTTCATCAAACTTTCTTCCCTCCCTAATC |
| | TCATTGGTTACCTTGGGCTATCGAAACTTAATTAAGCGATCTGCATCTCAATTAGTCAGCAACCATA |
| | GTCCCGCCCCTAACTCCGCCCATCCCGCCCCTAACTCCGCCCAGTTCCGCCCATTCTCCGCCCCATC |
| | GCTGACTAATTTTTTTTATTTATGCAGAGGCCGAGGCCGCCTCGGCCTCTGAGCTATTCCAGAAGTA |
| | GTGAGGAGGCTTTTTTGGAGGCCTAGGCTTTTGCAAAGGAGGTAGCCAACATGATTGAACAAGATGG |
| | ATTGCACGCAGGTTCTCCCGCCGCTTGGGTGGAGAGGCTATTCGGCTATGACTGGGCACAACAGACA |
| | ATCGGCTGCTCTGATGCCGCCGTGTTCCGGCTGTCAGCGCAGGGGCGCCCGGTTCTTTTTGTCAAGA |
| | CCGACCTGTCCGGTGCCCTGAATGAACTCCAGGACGAGGCAGCGCGGCTATCGTGGCTGGCCACGAC |
| | GGGCGTTCCTTGCGCAGCTGTGCTCGACGTTGTCACTGAAGCGGGAAGGGACTGGCTGCTATTGGGC |
| | GAAGTGCCGGGGCAGGATCTCCTGTCATCTCACCTTGCTCCTGCCGAGAAAGTATCCATCATGGCTG |
| | ATGCAATGCGGCGGCTGCATACGCTTGATCCGGCTACCTGCCCATTCGACCACCAAGCGAAACATCG |
| | CATCGAGCGAGCACGTACTCGGATGGAAGCCGGTCTTGTCGATCAGGATGATCTGGACGAAGAGCAT |
| | CAGGGGCTCGCGCCAGCCGAACTGTTCGCCAGGCTCAAGGCGCGGATGCCCGACGGCGAGGATCTCG |
| | TCGTGACCCACGGCGATGCCTGCTTGCCGAATATCATGGTGGAAAATGGCCGCTTTTCTGGATTCAT |
| | CGACTGTGGCCGGCTGGGTGTGGCGGACCGCTATCAGGACATAGCGTTGGCTACCCGTGATATTGCT |
| | GAAGAGCTTGGCGGCGAATGGGCTGACCGCTTCCTCGTGCTTTACGGTATCGCCGCTCCCGATTCGC |
| | AGCGCATCGCCTTCTATCGCCTTCTTGACGAGTTCTTCTAGTATGTAAGCCCTGTGCCTTCTAGTTG |
| | CCAGCCATCTGTTGTTTGCCCCTCCCCCGTGCCTTCCTTGACCCTGGAAGGTGCCACTCCCACTGTC |
| | CTTTCCTAATAAAATGAGGAAATTGCATCGCATTGTCTGAGTAGGTGTCATTCTATTCTGGGGGGTG |
| | GGGTGGGGCAGGACAGCAAGGGGGAGGATTGGGAAGACAATAGCAGGCATGCTGGGGATGCGGTGGG |
| | CTCTATGGTTAATTAACCAGTCAAGTCAGCTACTTGGCGAGATCGACTTGTCTGGGTTTCGACTACG |
| | CTCAGAATTGCGTCAGTCAAGTTCGATCTGGTCCTTGCTATTGCACCCGTTCTCCGATTACGAGTTT |
| | CATTTAAATCATGTGAGCAAAAGGCCAGCAAAAGGCCAGGAACCGTAAAAAGGCCGCGTTGCTGGCG |
| | TTTTTCCATAGGCTCCGCCCCCCTGACGAGCATCACAAAAATCGACGCTCAAGTCAGAGGTGGCGAA |
| | ACCCGACAGGACTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGCTTTCTCATAGC |
| | TCACGCTGTAGGTATCTCAGTTCGGTGTAGGTCGTTCGCTCCAAGCTGGGCTGTGTGCACGAACCCC |
| | CCGTTCAGCCCGACCGCTGCGCCTTATCCGGTAACTATCGTCTTGAGTCCAACCCGGTAAGACACGA |
| | CTTATCGCCACTGGCAGCAGCCACTGGTAACAGGATTAGCAGAGCGAGGTATGTAGGCGGTGCTACA |
| | GAGTTCTTGAAGTGGTGGCCTAACTACGGCTACACTAGAAGAACAGTATTTGGTATCTGCGCTCTGC |
| | TGAAGCCAGTTACCTTCGGAAAAAGAGTTGGTAGCTCTTGATCCGGCAAACAAACCACCGCTGGTAG |
| | CGGTGGTTTTTTTGTTTGCAAGCAGCAGATTACGCGCAGAAAAAAAGGATCTCAAGAAGATCCTTTG |
| | ATCTTTTCTACGGGGTCTGACGCTCAGTGGAACGAAAACTCACGTTAAGGGATTTTGGTCATGAGAT |
| | TATCAAAAAGGATCTTCACCTAGATCCTTTTAAATTAAAAATGAAGTTTTAAATCAATCTAAAGTAT |
| | ATATGAGTAAACTTGGTCTGACAGTTACCAATGCTTAATCAGTGAGGCACCTATCTCAGCGATCTGT |
| | CTATTTCGTTCATCCATAGTTGCATTTAAATTTCCGAACTCTCCAAGGCCCTCGTCGGAAAATCTTC |
| | AAACCTTTCGTCCGATCCATCTTGCAGGCTACCTCTCGAACGAACTATCGCAAGTCTCTTGGCCGGC |
| | CTTGCGCCTTGGCTATTGCTTGGCAGCGCCTATCGCCAGGTATTACTCCAATCCCGAATATCCGAGA |
| | TCGGGATCACCCGAGAGAAGTTCAACCTACATCCTCAATCCCGATCTATCCG (SEQ ID NO: |
| | 103) |
| RET- | AGAACGATCCTCTCAGTGCGAGTCTCGACGATCCATATCGTTGCTTGGCAGTCAGCCAGTCGGAATC |
| 014 | CAGCTTGGGACCCAGGAAGTCCAATCGTCAGATATTGTACTCAAGCCTGGTCACGGCAGCGTACCGA |
| LINE1- | TCTGTTTAAACCTAGATATTGATAGTCTGATCGGTCAACGTATAATCGAGTCCTAGCTTTTGCAAAC |
| GFP | ATCTATCAAGAGACAGGATCAGCAGGAGGCTTTCGCATGAGTATTCAACATTTCCGTGTCGCCCTTA |
| ORF2- | TTCCCTTTTTTGCGGCATTTTGCCTTCCTGTTTTTGCTCACCCAGAAACGCTGGTGAAAGTAAAAGA |
| CSV40- | TGCTGAAGATCAGTTGGGTGCGCGAGTGGGTTACATCGAACTGGATCTCAACAGCGGTAAGATCCTT |
| NLS | GAGAGTTTTCGCCCCGAAGAACGCTTTCCAATGATGAGCACTTTTAAAGTTCTGCTATGTGGCGCGG |
| | TATTATCCCGTATTGACGCCGGGCAAGAGCAACTCGGTCGCCGCATACACTATTCTCAGAATGACTT |
| | GGTTGAGTATTCACCAGTCACAGAAAAGCATCTTACGGATGGCATGACAGTAAGAGAATTATGCAGT |
| | GCTGCCATAACCATGAGTGATAACACTGCGGCCAACTTACTTCTGACAACGATTGGAGGACCGAAGG |
| | AGCTAACCGCTTTTTTGCACAACATGGGGGATCATGTAACTCGCCTTGATCGTTGGGAACCGGAGCT |
| | GAATGAAGCCATACCAAACGACGAGCGTGACACCACGATGCCTGTAGCAATGGCAACAACCTTGCGT |
| | AAACTATTAACTGGCGAACTACTTACTCTAGCTTCCCGGCAACAGTTGATAGACTGGATGGAGGCGG |
| | ATAAAGTTGCAGGACCACTTCTGCGCTCGGCCCTTCCGGCTGGCTGGTTTATTGCTGATAAATCTGG |
| | AGCCGGTGAGCGTGGGTCTCGCGGTATCATTGCAGCACTGGGGCCAGATGGTAAGCCCTCCCGTATC |
| | GTAGTTATCTACACGACGGGGAGTCAGGCAACTATGGATGAACGAAATAGACAGATCGCTGAGATAG |
| | GTGCCTCACTGATTAAGCATTGGTAACCGATTCTAGGTGCATTGGCGCAGAAAAAAATGCCTGATGC |
| | GACGCTGCGCGTCTTATACTCCCACATATGCCAGATTCAGCAACGGATACGGCTTCCCCAACTTGCC |
| | CACTTCCATACGTGTCCTCCTTACCAGAAATTTATCCTTAAGATCGTTTAAACTCGACTCTGGCTCT |
| | ATCGAATCTCCGTCGTTTCGAGCTTACGCGAACAGCCGTGGCGCTCATTTGCTCGTCGGGCATCGAA |
| | TCTCGTCAGCTATCGTCAGCTTACCTTTTTGGCAGCGATCGCGGCTCCCGACATCTTGGACCATTAG |
| | CTCCACAGGTATCTTCTTCCCTCTAGTGGTCATAACAGCAGCTTCAGCTACCTCTCAATTCAAAAAA |
| | CCCCTCAAGACCCGTTTAGAGGCCCCAAGGGGTTATGCTATCAATCGTTGCGTTACACACACAAAAA |
| | ACCAACACACATCCATCTTCGATGGATAGCGATTTTATTATCTAACTGCTGATCGAGTGTAGCCAGA |
| | TCTAGTAATCAATTACGGGGTCATTAGTTCATAGCCCATATATGGAGTTCCGCGTTACATAACTTAC |
| | GGTAAATGGCCCGCCTGGCTGACCGCCCAACGACCCCCGCCCATTGACGTCAATAATGACGTATGTT |
| | CCCATAGTAACGCCAATAGGGACTTTCCATTGACGTCAATGGGTGGAGTATTTACGGTAAACTGCCC |
| | ACTTGGCAGTACATCAAGTGTATCATATGCCAAGTACGCCCCCTATTGACGTCAATGACGGTAAATG |
| | GCCCGCCTGGCATTATGCCCAGTACATGACCTTATGGGACTTTCCTACTTGGCAGTACATCTACGTA |
| | TTAGTCATCGCTATTACCATGCTGATGCGGTTTTGGCAGTACATCAATGGGCGTGGATAGCGGTTTG |
| | ACTCACGGGGATTTCCAAGTCTCCACCCCATTGACGTCAATGGGAGTTTGTTTTGGCACCAAAATCA |
| | ACGGGACTTTCCAAAATGTCGTAACAACTCCGCCCCATTGACGCAAATGGGCGGTAGGCGTGTACGG |
| | TGGGAGGTCTATATAAGCAGAGCTGGTTTAGTGAACCGTCAGATCAGATCTTTGTCGATCCTACCAT |
| | CCACTCGACACACCCGCCAGCGGCCGCTAATACGACTCACTATAGGGAGAAGTACTGCCACCATGGG |
| | CAAGAAGCAAAATCGCAAGACGGGGAATTCCAAGACACAATCCGCTAGCCCACCACCTAAAGAGCGT |
| | TCTAGCTCCCCTGCTACTGAGCAGTCCTGGATGGAAAACGACTTCGATGAACTCCGGGAAGAGGGAT |
| | TTAGGCGATCCAACTATTCAGAACTCCGCGAAGATATCCAGACAAAGGGGAAGGAAGTCGAGAATTT |
| | CGAGAAGAACCTCGAGGAGTGCATCACCCGTATCACAAACACTGAGAAATGTCTCAAAGAACTCATG |
| | GAACTTAAGACAAAAGCCAGGGAGCTTCGAGAGGAGTGTCGGAGTCTGAGATCCAGGTGTGACCAGC |
| | TCGAGGAGCGCGTGAGCGCGATGGAAGACGAGATGAACGAGATGAAAAGAGAGGGCAAATTCAGGGA |
| | GAAGCGCATTAAGAGGAACGAACAGAGTCTGCAGGAGATTTGGGATTACGTCAAGAGGCCTAACCTG |
| | CGGTTGATCGGCGTCCCCGAGAGCGACGTAGAAAACGGGACTAAACTGGAGAATACACTTCAAGACA |
| | TCATTCAAGAAAATTTTCCAAACCTGGCTCGGCAAGCTAATGTGCAAATCCAAGAGATCCAACGCAC |
| | ACCCCAGCGGTATAGCTCTCGGCGTGCCACCCCTAGGCATATTATCGTGCGCTTTACTAAGGTGGAG |
| | ATGAAAGAGAAGATGCTGCGAGCCGCTCGGGAAAAGGGAAGGGTGACTTTGAAGGGCAAACCTATTC |
| | GGCTGACGGTTGACCTTAGCGCCGAGACACTCCAGGCACGCCGGGAATGGGGCCCCATCTTTAATAT |
| | CCTGAAGGAGAAGAACTTCCAGCCACGAATCTCTTACCCTGCAAAGTTGAGTTTTATCTCCGAGGGT |
| | GAGATTAAGTATTTCATCGATAAACAGATGCTGCGAGACTTCGTGACAACTCGCCCAGCTCTCAAGG |
| | AACTGCTCAAAGAGGCTCTTAATATGGAGCGCAATAATAGATATCAACCCTTGCAGAACCACGCAAA |
| | GATGTGAGACAGCCGTCAGACCATCAAGACTAGGAAGAAACTGCATCAACTAATGAGCAAAATCACC |
| | AGCTAACATCATAGTATACATGACCGGCTCTAACTCACATATCACCATCCTTACACTTAACATTAAC |
| | GGCCTCAACTCAGCTATCAAGCGCCATCGGCTGGCCAGCTGGATCAAATCACAGGATCCAAGCGTTT |
| | GTTGCATCCAAGAGACCCACCTGACCTGTAGAGATACTCACCGCCTCAAGATCAAGGGATGGCGAAA |
| | GATTTATCAGGCGAACGGTAAGCAGAAGAAAGCCGGAGTCGCAATTCTGGTCTCAGACAAGACGGAT |
| | TTCAAGCCCACCAAAATTAAGCGTGATAAGGAAGGTCACTATATTATGGTGAAAGGCAGCATACAGC |
| | AGGAAGAACTTACCATATTGAACATCTACGCGCCAAACACCGGCGCACCTCGCTTTATCAAACAGGT |
| | CCTGTCCGATCTGCAGCGAGATCTGGATTCTCATACGTTGATTATGGGTGATTTCAATACACCATTG |
| | AGCACCCTGGATCGCAGCACCAGGCAAAAGGTAAATAAAGACACGCAAGAGCTCAATAGCGCACTGC |
| | ATCAGGCAGATCTCATTGATATTTATCGCACTCTTCATCCTAAGAGTACCGAGTACACATTCTTCAG |
| | CGCCCCACATCATACATACTCAAAGATCGATCATATCGTCGGCTCAAAGGCTCTGCTGTCAAAGTGC |
| | AAGCGCACAGAGATAATTACAAATTACCTGTCAGATCATAGCGCGATCAAGCTCGAGCTGAGAATCA |
| | AGAACCTGACCCAGAGCCGGAGTACCACTTGGAAGCTTAATAACCTGCTGCTCAACGATTATTGGGT |
| | CCACAATGAGATGAAGGGAGAGATTAAAATGTTCTTCGAAACAAATGAGAATAAGGATACTACCTAT |
| | CAAAACCTTTGGGATGCCTTTAAGGCCGTCTGCAGAGGCAAGTTCATCGCCCTCAACGCCTATAAAA |
| | GAAAACAAGAGAGATCTAAGATCGATACTCTCACCTCTCAGCTGAAGGAGTTGGAGAAACAGGAACA |
| | GACCCACTCCAAGGCGTCAAGACGGCAGGAGATCACAAAGATTCGCGCCGAGTTGAAAGAGATCGAA |
| | ACCCAAAAGACTCTTCAGAAAATTAACGAGTCTCGTAGTTGGTTCTTCGAGCGGATTAATAAGATAG |
| | ACAGACCTCTGGCACGACTGATTAAGAAGAAGCGCGAAAAGAACCAGATTGATACCATCAAGAACGA |
| | CAAGGGCGACATCACTACTGACCCGACCGAGATCCAGACCACTATTCGGGAGTATTATAAGCATTTG |
| | TATGCTAACAAGCTTGAGAACCTGGAAGAGATGGACACTTTTCTGGATACCTATACTCTGCCACGGC |
| | TTAATCAAGAGGAAGTCGAGTCCCTCAACCGCCCAATTACAGGAAGCGAGATTGTGGCCATAATTAA |
| | CTCCCTGCCGACAAAGAAATCTCCTGGTCCGGACGGGTTTACAGCTGAGTTTTATCAACGGTATATG |
| | GAAGAGCTTGTACCGTTTCTGCTCAAGCTCTTTCAGTCTATAGAAAAGGAAGGCATCTTGCCCAATT |
| | CCTTCTACGAAGCTTCTATAATACTTATTCCCAAACCAGGACGCGATACCACAAAGAAGGAAAACTT |
| | CCGGCCCATTAGTCTCATGAATATCGACGCTAAAATATTGAACAAGATTCTCGCCAACAGAATCCAA |
| | CAACATATTAAGAAATTGATACATCACGACCAGGTGGGGTTTATACCTGGCATGCAGGGCTGGTTTA |
| | ACATCCGGAAGAGTATTAACGTCATTCAACACATTAATAGAGCTAAGGATAAGAATCATATGATCAT |
| | CTCTATAGACGCGGAAAAGGCATTCGATAAGATTCAGCAGCCATTTATGCTCAAGACTCTGAACAAA |
| | CTCGGCATCGACGGAACATATTTTAAGATTATTCGCGCAATTTACGATAAGCCGACTGCTAACATTA |
| | TCCTTAACGGCCAAAAGCTCGAGGCCTTTCCGCTCAAGACTGGAACCCGCCAAGGCTGTCCCCTCTC |
| | CCCGCTTTTGTTTAATATTGTACTCGAGGTGCTGGCTAGGGCTATTCGTCAAGAGAAAGAGATTAAA |
| | GGGATACAGCTCGGGAAGGAAGAGGTCAAGCTTTCCTTGTTCGCCGATGATATGATTGTGTACCTGG |
| | AGAATCCTATTGTGTCTGCTCAGAACCTTCTTAAACTTATTTCTAACTTTAGCAAGGTCAGCGGCTA |
| | TAAGATTAACGTCCAGAAATCTCAGGCCTTTCTGTACACAAATAATCGACAGACCGAATCCCAGATA |
| | ATGGGTGAGCTTCCGTTTGTCATAGCCAGCAAAAGGATAAAGTATCTCGGAATCCAGCTGACACGAG |
| | ACGTTAAAGATTTGTTTAAGGAAAATTACAAGCCTCTCCTGAAAGAGATTAAGGAAGATACTAATAA |
| | GTGGAAGAATATCCCCTGTTCATGGGTTGGCAGAATCAACATAGTGAAGATGGCAATACTTCCTAAA |
| | GTGATATATCGCTTTAACGCCATCCCAATTAAACTGCCTATGACCTTCTTTACGGAGCTCGAGAAAA |
| | CAACCCTTAAATTTATATGGAATCAAAAGAGAGCAAGAATAGCGAAGTCCATCTTGAGCCAGAAGAA |
| | TAAGGCCGGTGGGATTACTTTGCCTGATTTTAAGTTGTATTATAAAGCCACAGTAACTAAGACAGCC |
| | TGGTATTGGTATCAGAATAGAGACATCGACCAGTGGAATCGGACCGAACCATCAGAGATAATGCCCC |
| | ACATCTATAATTACCTTATATTCGATAAGCCAGAAAAGAATAAACAGTGGGGCAAAGACAGCCTCTT |
| | CAACAAGTGGTGTTGGGAGAATTGGCTGGCCATATGCCGGAAACTCAAGCTCGACCCCTTTCTTACA |
| | CCCTACACTAAAATCAACAGTAGGTGGATCAAGGACTTGAATGTCAAGCCAAAGACTATAAAGACAC |
| | TGGAAGAGAATCTTGGGATCACAATACAAGATATAGGCGTCGGCAAAGATTTTATGTCAAAGACGCC |
| | CAAGGCCATGGCCACTAAGGATAAGATTGATAAGTGGGACCTTATTAAGCTCAAAAGCTTCTGTACT |
| | GCCAAGGAGACCACGATCAGAGTTAATAGGCAGCCCACTACATGGGAAAAGATTTTCGCCACTTATT |
| | CATCAGATAAGGGGTTGATAAGCAGAATATATAACGAGCTGAAGCAGATCTACAAGAAGAAAACGAA |
| | TAATCCCATCAAGAAGTGGGCAAAAGATATGAACAGGCATTTTAGCAAAGAGGATATCTACGCCGCG |
| | AAGAAGCATATGAAGAAGTGTAGTTCAAGCTTGGCCATTCGTGAGATGCAGATTAAGACGACCATGC |
| | GATACCACCTTACCCCAGTGAGGATGGCAATTATCAAGAAATCTGGCAATAATAGATGTTGGCGGGG |
| | CTGTGGCGAGATTGGCACCCTGCTCCATTGCTGGTGGGATTGCAAGCTGGTGCAGCCGCTTTGGAAA |
| | TCAGTCTGGCGCTTTCTGAGGGACCTCGAGCTTGAGATTCCCTTCGATCCCGCAATTCCCTTGCTCG |
| | GAATCTATCCTAACGAATACAAGAGCTGTTGTTACAAGGATACGTGTACCCGGATGTTCATCGCGGC |
| | CTTGTTTACGATAGCTAAGACGTGGAATCAGCCTAAGTGCCCCACAATGATCGATTGGATCAAGAAA |
| | ATGTGGCATATTTATACCATGGAGTATTACGCAGCAATTAAGAATGACGAATTTATTTCCTTCGTTG |
| | GGACCTGGATGAAGCTGGAGACTATTATTCTGAGCAAGCTGTCTCAGGAGCAAAAGACAAAGCATAG |
| | AATCTTCTCTCTCATTGGTGGTAACGACTACAAAGACGATGACGACAAGccaaagaagaagcggaag |
| | gtcTAAAGCGCTTCTAGAAGTTGTCTCCTCCTGCACTGACTGACTGATACAATCGATTTCTGGATCC |
| | GCAGGCCTAATCAACCTCTGGATTACAAAATTTGTGAAAGATTGACTGGTATTCTTAACTATGTTGC |
| | TCCTTTTACGCTATGTGGATACGCTGCTTTAATGCCTTTGTATCATGCTATTGCTTCCCGTATGGCT |
| | TTCATTTTCTCCTCCTTGTATAAATCCTGGTTGCTGTCTCTTTATGAGGAGTTGTGGCCCGTTGTCA |
| | GGCAACGTGGCGTGGTGTGCACTGTGTTTGCTGACGCAACCCCCACTGGTTGGGGCATTGCCACCAC |
| | CTGTCAGCTCCTTTCCGGGACTTTCGCTTTCCCCCTCCCTATTGCCACGGCGGAACTCATCGCCGCC |
| | TGCCTTGCCCGCTGCTGGACAGGGGCTCGGCTGTTGGGCACTGACAATTCCGTGGTGTTGTCGGGGA |
| | AGCTGACGTCCTTTCCATGGCTGCTCGCCTGTGTTGCCACCTGGATTCTGCGCGGGACGTCCTTCTG |
| | CTACGTCCCTTCGGCCCTCAATCCAGCGGACCTTCCTTCCCGCtgagagacacaaaaaattccaaca |
| | cactattgcaatgaaaataaatttcctttattagccagaagtcagatgctcaaggggcttcatgatg |
| | tccccataatttttggcagagggaaaaagatctcagtggtatttgtgagccagggcattggccttct |
| | gataggcagcctgcacctgaggagtgcggccgctttacttgtacagctcgtccatgccgagagtgat |
| | cccggcggcggtcacgaactccagcaggaccatgtgatcgcgcttctcgttggggtctttgctcagg |
| | gcggactgggtgctcaggtagtggttgtcgggcagcagcacggggccgtcgccgatgggggtgttct |
| | gctggtagtggtcggcgagctgcacgctgccgtcctcgatgttgtggcggatcttgaagttcacctt |
| | gatgccgttcttctgcttgtcggccatgatatagacgttgtggctgttgtagttgtactccagcttg |
| | tgccccaggatgttgccgtcctccttgaagtcgatgcccttcagctcgatgcggttcaccagggtgt |
| | cgccctcgaacttcacctcggcgcgggtcttgtagttgccgtcgtccttgaagaagatggtgcgctc |
| | ctggacgtagccttcgggcatggcggacttgaagaagtcgtgctgcttcatgtggtcggggtagcgg |
| | ctgaagcactgcacgccgtaggtcagggtggtcacgagggtgggccagggcacgggcagcttgccgg |
| | tggtgcagatgaacttcagggtcagcttgccgtaggtggcatcgccctcgccctcgccggacacgct |
| | gaacttgtggccgtttacgtcgccgtccagctcgaccaggatgggcaccaccccggtgaacagctcc |
| | tcgcccttgctcaccatggtggcgggatctgacggttcactaaaccagctctgcttatatagacctc |
| | ccaccgtacacgcctaccgcccatttgcgtcaatggggcggagttgttacgacattttggaaagtcc |
| | cgttgattttggtgccaaaacaaactcccattgacgtcaatggggtggagacttggaaatccccgtg |
| | agtcaaaccgctatccacgcccattgatgtactgccaaaaccgcatcaccatggtaatagcgatgac |
| | taatacgtagatgtactgccaagtaggaaagtcccataaggtcatgtactgggcataatgccaggcg |
| | ggccatttaccgtcattgacgtcaatagggggcgtacttggcatatgatacacttgatgtactgcca |
| | agtgggcagtttaccgtaaatactccacccattgacgtcaatggaaagtccctattggcgttactat |
| | gggaacatacgtcattattgacgtcaatgggcgggggtcgttgggcggtcagccaggcgggccattt |
| | accgtaagttatgtaacgGGCCTGCTGCCGGCTCTGCGGCCTCTTCCGCGTCTTCGCCTTCGCCCTC |
| | AGACGAGTCGGATCTCCCTTTGGGCCGCCTCCCCGCCTGTCTAGCTTGACTGACTGAGATACAGCGT |
| | ACCTTCAGCTCACAGACATGATAAGATACATTGATGAGTTTGGACAAACCACAACTAGAATGCAGTG |
| | AAAAAAATGCTTTATTTGTGAAATTTGTGATGCTATTGCTTTATTTGTAACCATTATAAGCTGCAAT |
| | AAACAAGTTAACAACAACAATTGCATTCATTTTATGTTTCAGGTTCAGGGGGAGGTGTGGGAGGTTT |
| | TTTAAAGCAAGTAAAACCTCTACAAATGTGGTATTGGCCCATCTCTATCGGTATCGTAGCATAACCC |
| | CTTGGGGCCTCTAAACGGGTCTTGAGGGGTTTTTTGTGCCCCTCGGGCCGGATTGCTATCTACCGGC |
| | ATTGGCGCAGAAAAAAATGCCTGATGCGACGCTGCGCGTCTTATACTCCCACATATGCCAGATTCAG |
| | CAACGGATACGGCTTCCCCAACTTGCCCACTTCCATACGTGTCCTCCTTACCAGAAATTTATCCTTA |
| | AGGTCGTCAGCTATCCTGCAGGCGATCTCTCGATTTCGATCAAGACATTCCTTTAATGGTCTTTTCT |
| | GGACACCACTAGGGGTCAGAAGTAGTTCATCAAACTTTCTTCCCTCCCTAATCTCATTGGTTACCTT |
| | GGGCTATCGAAACTTAATTAAGCGATCTGCATCTCAATTAGTCAGCAACCATAGTCCCGCCCCTAAC |
| | TCCGCCCATCCCGCCCCTAACTCCGCCCAGTTCCGCCCATTCTCCGCCCCATCGCTGACTAATTTTT |
| | TTTATTTATGCAGAGGCCGAGGCCGCCTCGGCCTCTGAGCTATTCCAGAAGTAGTGAGGAGGCTTTT |
| | TTGGAGGCCTAGGCTTTTGCAAAGGAGGTAGCCAACATGATTGAACAAGATGGATTGCACGCAGGTT |
| | CTCCCGCCGCTTGGGTGGAGAGGCTATTCGGCTATGACTGGGCACAACAGACAATCGGCTGCTCTGA |
| | TGCCGCCGTGTTCCGGCTGTCAGCGCAGGGGCGCCCGGTTCTTTTTGTCAAGACCGACCTGTCCGGT |
| | GCCCTGAATGAACTCCAGGACGAGGCAGCGCGGCTATCGTGGCTGGCCACGACGGGCGTTCCTTGCG |
| | CAGCTGTGCTCGACGTTGTCACTGAAGCGGGAAGGGACTGGCTGCTATTGGGCGAAGTGCCGGGGCA |
| | GGATCTCCTGTCATCTCACCTTGCTCCTGCCGAGAAAGTATCCATCATGGCTGATGCAATGCGGCGG |
| | CTGCATACGCTTGATCCGGCTACCTGCCCATTCGACCACCAAGCGAAACATCGCATCGAGCGAGCAC |
| | GTACTCGGATGGAAGCCGGTCTTGTCGATCAGGATGATCTGGACGAAGAGCATCAGGGGCTCGCGCC |
| | AGCCGAACTGTTCGCCAGGCTCAAGGCGCGGATGCCCGACGGCGAGGATCTCGTCGTGACCCACGGC |
| | GATGCCTGCTTGCCGAATATCATGGTGGAAAATGGCCGCTTTTCTGGATTCATCGACTGTGGCCGGC |
| | TGGGTGTGGCGGACCGCTATCAGGACATAGCGTTGGCTACCCGTGATATTGCTGAAGAGCTTGGCGG |
| | CGAATGGGCTGACCGCTTCCTCGTGCTTTACGGTATCGCCGCTCCCGATTCGCAGCGCATCGCCTTC |
| | TATCGCCTTCTTGACGAGTTCTTCTAGTATGTAAGCCCTGTGCCTTCTAGTTGCCAGCCATCTGTTG |
| | TTTGCCCCTCCCCCGTGCCTTCCTTGACCCTGGAAGGTGCCACTCCCACTGTCCTTTCCTAATAAAA |
| | TGAGGAAATTGCATCGCATTGTCTGAGTAGGTGTCATTCTATTCTGGGGGGTGGGGTGGGGCAGGAC |
| | AGCAAGGGGGAGGATTGGGAAGACAATAGCAGGCATGCTGGGGATGCGGTGGGCTCTATGGTTAATT |
| | AACCAGTCAAGTCAGCTACTTGGCGAGATCGACTTGTCTGGGTTTCGACTACGCTCAGAATTGCGTC |
| | AGTCAAGTTCGATCTGGTCCTTGCTATTGCACCCGTTCTCCGATTACGAGTTTCATTTAAATCATGT |
| | GAGCAAAAGGCCAGCAAAAGGCCAGGAACCGTAAAAAGGCCGCGTTGCTGGCGTTTTTCCATAGGCT |
| | CCGCCCCCCTGACGAGCATCACAAAAATCGACGCTCAAGTCAGAGGTGGCGAAACCCGACAGGACTA |
| | TAAAGATACCAGGCGTTTCCCCCTGGAAGCTCCCTCGTGCGCTCTCCTGTTCCGACCCTGCCGCTTA |
| | CCGGATACCTGTCCGCCTTTCTCCCTTCGGGAAGCGTGGCGCTTTCTCATAGCTCACGCTGTAGGTA |
| | TCTCAGTTCGGTGTAGGTCGTTCGCTCCAAGCTGGGCTGTGTGCACGAACCCCCCGTTCAGCCCGAC |
| | CGCTGCGCCTTATCCGGTAACTATCGTCTTGAGTCCAACCCGGTAAGACACGACTTATCGCCACTGG |
| | CAGCAGCCACTGGTAACAGGATTAGCAGAGCGAGGTATGTAGGCGGTGCTACAGAGTTCTTGAAGTG |
| | GTGGCCTAACTACGGCTACACTAGAAGAACAGTATTTGGTATCTGCGCTCTGCTGAAGCCAGTTACC |
| | TTCGGAAAAAGAGTTGGTAGCTCTTGATCCGGCAAACAAACCACCGCTGGTAGCGGTGGTTTTTTTG |
| | TTTGCAAGCAGCAGATTACGCGCAGAAAAAAAGGATCTCAAGAAGATCCTTTGATCTTTTCTACGGG |
| | GTCTGACGCTCAGTGGAACGAAAACTCACGTTAAGGGATTTTGGTCATGAGATTATCAAAAAGGATC |
| | TTCACCTAGATCCTTTTAAATTAAAAATGAAGTTTTAAATCAATCTAAAGTATATATGAGTAAACTT |
| | GGTCTGACAGTTACCAATGCTTAATCAGTGAGGCACCTATCTCAGCGATCTGTCTATTTCGTTCATC |
| | CATAGTTGCATTTAAATTTCCGAACTCTCCAAGGCCCTCGTCGGAAAATCTTCAAACCTTTCGTCCG |
| | ATCCATCTTGCAGGCTACCTCTCGAACGAACTATCGCAAGTCTCTTGGCCGGCCTTGCGCCTTGGCT |
| | ATTGCTTGGCAGCGCCTATCGCCAGGTATTACTCCAATCCCGAATATCCGAGATCGGGATCACCCGA |
| | GAGAAGTTCAACCTACATCCTCAATCCCGATCTATCCGAGATCCGAGGAATATCGAAATCGGGGCGC |
| | GCCTGGTGTACCG (SEQ ID NO: 104) |
| RET- | TCGACGATCCATATCGTTGCTTGGCAGTCAGCCAGTCGGAATCCAGCTTGGGACCCAGGAAGTCCAA |
| 015 | TCGTCAGATATTGTACTCAAGCCTGGTCACGGCAGCGTACCGATCTGTTTAAACCTAGATATTGATA |
| LINE1- | GTCTGATCGGTCAACGTATAATCGAGTCCTAGCTTTTGCAAACATCTATCAAGAGACAGGATCAGCA |
| GFP | GGAGGCTTTCGCATGAGTATTCAACATTTCCGTGTCGCCCTTATTCCCTTTTTTGCGGCATTTTGCC |
| ORF2- | TTCCTGTTTTTGCTCACCCAGAAACGCTGGTGAAAGTAAAAGATGCTGAAGATCAGTTGGGTGCGCG |
| C_Linker_ | AGTGGGTTACATCGAACTGGATCTCAACAGCGGTAAGATCCTTGAGAGTTTTCGCCCCGAAGAACGC |
| SV40- | TTTCCAATGATGAGCACTTTTAAAGTTCTGCTATGTGGCGCGGTATTATCCCGTATTGACGCCGGGC |
| NLS | AAGAGCAACTCGGTCGCCGCATACACTATTCTCAGAATGACTTGGTTGAGTATTCACCAGTCACAGA |
| | AAAGCATCTTACGGATGGCATGACAGTAAGAGAATTATGCAGTGCTGCCATAACCATGAGTGATAAC |
| | ACTGCGGCCAACTTACTTCTGACAACGATTGGAGGACCGAAGGAGCTAACCGCTTTTTTGCACAACA |
| | TGGGGGATCATGTAACTCGCCTTGATCGTTGGGAACCGGAGCTGAATGAAGCCATACCAAACGACGA |
| | GCGTGACACCACGATGCCTGTAGCAATGGCAACAACCTTGCGTAAACTATTAACTGGCGAACTACTT |
| | ACTCTAGCTTCCCGGCAACAGTTGATAGACTGGATGGAGGCGGATAAAGTTGCAGGACCACTTCTGC |
| | GCTCGGCCCTTCCGGCTGGCTGGTTTATTGCTGATAAATCTGGAGCCGGTGAGCGTGGGTCTCGCGG |
| | TATCATTGCAGCACTGGGGCCAGATGGTAAGCCCTCCCGTATCGTAGTTATCTACACGACGGGGAGT |
| | CAGGCAACTATGGATGAACGAAATAGACAGATCGCTGAGATAGGTGCCTCACTGATTAAGCATTGGT |
| | AACCGATTCTAGGTGCATTGGCGCAGAAAAAAATGCCTGATGCGACGCTGCGCGTCTTATACTCCCA |
| | CATATGCCAGATTCAGCAACGGATACGGCTTCCCCAACTTGCCCACTTCCATACGTGTCCTCCTTAC |
| | CAGAAATTTATCCTTAAGATCGTTTAAACTCGACTCTGGCTCTATCGAATCTCCGTCGTTTCGAGCT |
| | TACGCGAACAGCCGTGGCGCTCATTTGCTCGTCGGGCATCGAATCTCGTCAGCTATCGTCAGCTTAC |
| | CTTTTTGGCAGCGATCGCGGCTCCCGACATCTTGGACCATTAGCTCCACAGGTATCTTCTTCCCTCT |
| | AGTGGTCATAACAGCAGCTTCAGCTACCTCTCAATTCAAAAAACCCCTCAAGACCCGTTTAGAGGCC |
| | CCAAGGGGTTATGCTATCAATCGTTGCGTTACACACACAAAAAACCAACACACATCCATCTTCGATG |
| | GATAGCGATTTTATTATCTAACTGCTGATCGAGTGTAGCCAGATCTAGTAATCAATTACGGGGTCAT |
| | TAGTTCATAGCCCATATATGGAGTTCCGCGTTACATAACTTACGGTAAATGGCCCGCCTGGCTGACC |
| | GCCCAACGACCCCCGCCCATTGACGTCAATAATGACGTATGTTCCCATAGTAACGCCAATAGGGACT |
| | TTCCATTGACGTCAATGGGTGGAGTATTTACGGTAAACTGCCCACTTGGCAGTACATCAAGTGTATC |
| | ATATGCCAAGTACGCCCCCTATTGACGTCAATGACGGTAAATGGCCCGCCTGGCATTATGCCCAGTA |
| | CATGACCTTATGGGACTTTCCTACTTGGCAGTACATCTACGTATTAGTCATCGCTATTACCATGCTG |
| | ATGCGGTTTTGGCAGTACATCAATGGGCGTGGATAGCGGTTTGACTCACGGGGATTTCCAAGTCTCC |
| | ACCCCATTGACGTCAATGGGAGTTTGTTTTGGCACCAAAATCAACGGGACTTTCCAAAATGTCGTAA |
| | CAACTCCGCCCCATTGACGCAAATGGGCGGTAGGCGTGTACGGTGGGAGGTCTATATAAGCAGAGCT |
| | GGTTTAGTGAACCGTCAGATCAGATCTTTGTCGATCCTACCATCCACTCGACACACCCGCCAGCGGC |
| | CGCTAATACGACTCACTATAGGGAGAAGTACTGCCACCATGGGCAAGAAGCAAAATCGCAAGACGGG |
| | GAATTCCAAGACACAATCCGCTAGCCCACCACCTAAAGAGCGTTCTAGCTCCCCTGCTACTGAGCAG |
| | TCCTGGATGGAAAACGACTTCGATGAACTCCGGGAAGAGGGATTTAGGCGATCCAACTATTCAGAAC |
| | TCCGCGAAGATATCCAGACAAAGGGGAAGGAAGTCGAGAATTTCGAGAAGAACCTCGAGGAGTGCAT |
| | CACCCGTATCACAAACACTGAGAAATGTCTCAAAGAACTCATGGAACTTAAGACAAAAGCCAGGGAG |
| | CTTCGAGAGGAGTGTCGGAGTCTGAGATCCAGGTGTGACCAGCTCGAGGAGCGCGTGAGCGCGATGG |
| | AAGACGAGATGAACGAGATGAAAAGAGAGGGCAAATTCAGGGAGAAGCGCATTAAGAGGAACGAACA |
| | GAGTCTGCAGGAGATTTGGGATTACGTCAAGAGGCCTAACCTGCGGTTGATCGGCGTCCCCGAGAGC |
| | GACGTAGAAAACGGGACTAAACTGGAGAATACACTTCAAGACATCATTCAAGAAAATTTTCCAAACC |
| | TGGCTCGGCAAGCTAATGTGCAAATCCAAGAGATCCAACGCACACCCCAGCGGTATAGCTCTCGGCG |
| | TGCCACCCCTAGGCATATTATCGTGCGCTTTACTAAGGTGGAGATGAAAGAGAAGATGCTGCGAGCC |
| | GCTCGGGAAAAGGGAAGGGTGACTTTGAAGGGCAAACCTATTCGGCTGACGGTTGACCTTAGCGCCG |
| | AGACACTCCAGGCACGCCGGGAATGGGGCCCCATCTTTAATATCCTGAAGGAGAAGAACTTCCAGCC |
| | ACGAATCTCTTACCCTGCAAAGTTGAGTTTTATCTCCGAGGGTGAGATTAAGTATTTCATCGATAAA |
| | CAGATGCTGCGAGACTTCGTGACAACTCGCCCAGCTCTCAAGGAACTGCTCAAAGAGGCTCTTAATA |
| | TGGAGCGCAATAATAGATATCAACCCTTGCAGAACCACGCAAAGATGTGAGACAGCCGTCAGACCAT |
| | CAAGACTAGGAAGAAACTGCATCAACTAATGAGCAAAATCACCAGCTAACATCATAGTATACATGAC |
| | CGGCTCTAACTCACATATCACCATCCTTACACTTAACATTAACGGCCTCAACTCAGCTATCAAGCGC |
| | CATCGGCTGGCCAGCTGGATCAAATCACAGGATCCAAGCGTTTGTTGCATCCAAGAGACCCACCTGA |
| | CCTGTAGAGATACTCACCGCCTCAAGATCAAGGGATGGCGAAAGATTTATCAGGCGAACGGTAAGCA |
| | GAAGAAAGCCGGAGTCGCAATTCTGGTCTCAGACAAGACGGATTTCAAGCCCACCAAAATTAAGCGT |
| | GATAAGGAAGGTCACTATATTATGGTGAAAGGCAGCATACAGCAGGAAGAACTTACCATATTGAACA |
| | TCTACGCGCCAAACACCGGCGCACCTCGCTTTATCAAACAGGTCCTGTCCGATCTGCAGCGAGATCT |
| | GGATTCTCATACGTTGATTATGGGTGATTTCAATACACCATTGAGCACCCTGGATCGCAGCACCAGG |
| | CAAAAGGTAAATAAAGACACGCAAGAGCTCAATAGCGCACTGCATCAGGCAGATCTCATTGATATTT |
| | ATCGCACTCTTCATCCTAAGAGTACCGAGTACACATTCTTCAGCGCCCCACATCATACATACTCAAA |
| | GATCGATCATATCGTCGGCTCAAAGGCTCTGCTGTCAAAGTGCAAGCGCACAGAGATAATTACAAAT |
| | TACCTGTCAGATCATAGCGCGATCAAGCTCGAGCTGAGAATCAAGAACCTGACCCAGAGCCGGAGTA |
| | CCACTTGGAAGCTTAATAACCTGCTGCTCAACGATTATTGGGTCCACAATGAGATGAAGGCAGAGAT |
| | TAAAATGTTCTTCGAAACAAATGAGAATAAGGATACTACCTATCAAAACCTTTGGGATGCCTTTAAG |
| | GCCGTCTGCAGAGGCAAGTTCATCGCCCTCAACGCCTATAAAAGAAAACAAGAGAGATCTAAGATCG |
| | ATACTCTCACCTCTCAGCTGAAGGAGTTGGAGAAACAGGAACAGACCCACTCCAAGGCGTCAAGACG |
| | GCAGGAGATCACAAAGATTCGCGCCGAGTTGAAAGAGATCGAAACCCAAAAGACTCTTCAGAAAATT |
| | AACGAGTCTCGTAGTTGGTTCTTCGAGCGGATTAATAAGATAGACAGACCTCTGGCACGACTGATTA |
| | AGAAGAAGCGCGAAAAGAACCAGATTGATACCATCAAGAACGACAAGGGCGACATCACTACTGACCC |
| | GACCGAGATCCAGACCACTATTCGGGAGTATTATAAGCATTTGTATGCTAACAAGCTTGAGAACCTG |
| | GAAGAGATGGACACTTTTCTGGATACCTATACTCTGCCACGGCTTAATCAAGAGGAAGTCGAGTCCC |
| | TCAACCGCCCAATTACAGGAAGCGAGATTGTGGCCATAATTAACTCCCTGCCGACAAAGAAATCTCC |
| | TGGTCCGGACGGGTTTACAGCTGAGTTTTATCAACGGTATATGGAAGAGCTTGTACCGTTTCTGCTC |
| | AAGCTCTTTCAGTCTATAGAAAAGGAAGGCATCTTGCCCAATTCCTTCTACGAAGCTTCTATAATAC |
| | TTATTCCCAAACCAGGACGCGATACCACAAAGAAGGAAAACTTCCGGCCCATTAGTCTCATGAATAT |
| | CGACGCTAAAATATTGAACAAGATTCTCGCCAACAGAATCCAACAACATATTAAGAAATTGATACAT |
| | CACGACCAGGTGGGGTTTATACCTGGCATGCAGGGCTGGTTTAACATCCGGAAGAGTATTAACGTCA |
| | TTCAACACATTAATAGAGCTAAGGATAAGAATCATATGATCATCTCTATAGACGCGGAAAAGGCATT |
| | CGATAAGATTCAGCAGCCATTTATGCTCAAGACTCTGAACAAACTCGGCATCGACGGAACATATTTT |
| | AAGATTATTCGCGCAATTTACGATAAGCCGACTGCTAACATTATCCTTAACGGCCAAAAGCTCGAGG |
| | CCTTTCCGCTCAAGACTGGAACCCGCCAAGGCTGTCCCCTCTCCCCGCTTTTGTTTAATATTGTACT |
| | CGAGGTGCTGGCTAGGGCTATTCGTCAAGAGAAAGAGATTAAAGGGATACAGCTCGGGAAGGAAGAG |
| | GTCAAGCTTTCCTTGTTCGCCGATGATATGATTGTGTACCTGGAGAATCCTATTGTGTCTGCTCAGA |
| | ACCTTCTTAAACTTATTTCTAACTTTAGCAAGGTCAGCGGCTATAAGATTAACGTCCAGAAATCTCA |
| | GGCCTTTCTGTACACAAATAATCGACAGACCGAATCCCAGATAATGGGTGAGCTTCCGTTTGTCATA |
| | GCCAGCAAAAGGATAAAGTATCTCGGAATCCAGCTGACACGAGACGTTAAAGATTTGTTTAAGGAAA |
| | ATTACAAGCCTCTCCTGAAAGAGATTAAGGAAGATACTAATAAGTGGAAGAATATCCCCTGTTCATG |
| | GGTTGGCAGAATCAACATAGTGAAGATGGCAATACTTCCTAAAGTGATATATCGCTTTAACGCCATC |
| | CCAATTAAACTGCCTATGACCTTCTTTACGGAGCTCGAGAAAACAACCCTTAAATTTATATGGAATC |
| | AAAAGAGAGCAAGAATAGCGAAGTCCATCTTGAGCCAGAAGAATAAGGCCGGTGGGATTACTTTGCC |
| | TGATTTTAAGTTGTATTATAAAGCCACAGTAACTAAGACAGCCTGGTATTGGTATCAGAATAGAGAC |
| | ATCGACCAGTGGAATCGGACCGAACCATCAGAGATAATGCCCCACATCTATAATTACCTTATATTCG |
| | ATAAGCCAGAAAAGAATAAACAGTGGGGCAAAGACAGCCTCTTCAACAAGTGGTGTTGGGAGAATTG |
| | GCTGGCCATATGCCGGAAACTCAAGCTCGACCCCTTTCTTACACCCTACACTAAAATCAACAGTAGG |
| | TGGATCAAGGACTTGAATGTCAAGCCAAAGACTATAAAGACACTGGAAGAGAATCTTGGGATCACAA |
| | TACAAGATATAGGCGTCGGCAAAGATTTTATGTCAAAGACGCCCAAGGCCATGGCCACTAAGGATAA |
| | GATTGATAAGTGGGACCTTATTAAGCTCAAAAGCTTCTGTACTGCCAAGGAGACCACGATCAGAGTT |
| | AATAGGCAGCCCACTACATGGGAAAAGATTTTCGCCACTTATTCATCAGATAAGGGGTTGATAAGCA |
| | GAATATATAACGAGCTGAAGCAGATCTACAAGAAGAAAACGAATAATCCCATCAAGAAGTGGGCAAA |
| | AGATATGAACAGGCATTTTAGCAAAGAGGATATCTACGCCGCGAAGAAGCATATGAAGAAGTGTAGT |
| | TCAAGCTTGGCCATTCGTGAGATGCAGATTAAGACGACCATGCGATACCACCTTACCCCAGTGAGGA |
| | TGGCAATTATCAAGAAATCTGGCAATAATAGATGTTGGCGGGGCTGTGGCGAGATTGGCACCCTGCT |
| | CCATTGCTGGTGGGATTGCAAGCTGGTGCAGCCGCTTTGGAAATCAGTCTGGCGCTTTCTGAGGGAC |
| | CTCGAGCTTGAGATTCCCTTCGATCCCGCAATTCCCTTGCTCGGAATCTATCCTAACGAATACAAGA |
| | GCTGTTGTTACAAGGATACGTGTACCCGGATGTTCATCGCGGCCTTGTTTACGATAGCTAAGACGTG |
| | GAATCAGCCTAAGTGCCCCACAATGATCGATTGGATCAAGAAAATGTGGCATATTTATACCATGGAG |
| | TATTACGCAGCAATTAAGAATGACGAATTTATTTCCTTCGTTGGGACCTGGATGAAGCTGGAGACTA |
| | TTATTCTGAGCAAGCTGTCTCAGGAGCAAAAGACAAAGCATAGAATCTTCTCTCTCATTGGTGGTAA |
| | CGACTACAAAGACGATGACGACAAGggcggcggcagcccaaagaagaagcggaaggtcTAAAGCGCT |
| | TCTAGAAGTTGTCTCCTCCTGCACTGACTGACTGATACAATCGATTTCTGGATCCGCAGGCCTAATC |
| | AACCTCTGGATTACAAAATTTGTGAAAGATTGACTGGTATTCTTAACTATGTTGCTCCTTTTACGCT |
| | ATGTGGATACGCTGCTTTAATGCCTTTGTATCATGCTATTGCTTCCCGTATGGCTTTCATTTTCTCC |
| | TCCTTGTATAAATCCTGGTTGCTGTCTCTTTATGAGGAGTTGTGGCCCGTTGTCAGGCAACGTGGCG |
| | TGGTGTGCACTGTGTTTGCTGACGCAACCCCCACTGGTTGGGGCATTGCCACCACCTGTCAGCTCCT |
| | TTCCGGGACTTTCGCTTTCCCCCTCCCTATTGCCACGGCGGAACTCATCGCCGCCTGCCTTGCCCGC |
| | TGCTGGACAGGGGCTCGGCTGTTGGGCACTGACAATTCCGTGGTGTTGTCGGGGAAGCTGACGTCCT |
| | TTCCATGGCTGCTCGCCTGTGTTGCCACCTGGATTCTGCGCGGGACGTCCTTCTGCTACGTCCCTTC |
| | GGCCCTCAATCCAGCGGACCTTCCTTCCCGCtgagagacacaaaaaattccaacacactattgcaat |
| | gaaaataaatttcctttattagccagaagtcagatgctcaaggggcttcatgatgtccccataattt |
| | ttggcagagggaaaaagatctcagtggtatttgtgagccagggcattggccttctgataggcagcct |
| | gcacctgaggagtgcggccgctttacttgtacagctcgtccatgccgagagtgatcccggcggcggt |
| | cacgaactccagcaggaccatgtgatcgcgcttctcgttggggtctttgctcagggcggactgggtg |
| | ctcaggtagtggttgtcgggcagcagcacggggccgtcgccgatgggggtgttctgctggtagtggt |
| | cggcgagctgcacgctgccgtcctcgatgttgtggcggatcttgaagttcaccttgatgccgttctt |
| | ctgcttgtcggccatgatatagacgttgtggctgttgtagttgtactccagcttgtgccccaggatg |
| | ttgccgtcctccttgaagtcgatgcccttcagctcgatgcggttcaccagggtgtcgccctcgaact |
| | tcacctcggcgcgggtcttgtagttgccgtcgtccttgaagaagatggtgcgctcctggacgtagcc |
| | ttcgggcatggcggacttgaagaagtcgtgctgcttcatgtggtcggggtagcggctgaagcactgc |
| | acgccgtaggtcagggtggtcacgagggtgggccagggcacgggcagcttgccggtggtgcagatga |
| | acttcagggtcagcttgccgtaggtggcatcgccctcgccctcgccggacacgctgaacttgtggcc |
| | gtttacgtcgccgtccagctcgaccaggatgggcaccaccccggtgaacagctcctcgcccttgctc |
| | accatggtggcgggatctgacggttcactaaaccagctctgcttatatagacctcccaccgtacacg |
| | cctaccgcccatttgcgtcaatggggcggagttgttacgacattttggaaagtcccgttgattttgg |
| | tgccaaaacaaactcccattgacgtcaatggggtggagacttggaaatccccgtgagtcaaaccgct |
| | atccacgcccattgatgtactgccaaaaccgcatcaccatggtaatagcgatgactaatacgtagat |
| | gtactgccaagtaggaaagtcccataaggtcatgtactgggcataatgccaggcgggccatttaccg |
| | tcattgacgtcaatagggggcgtacttggcatatgatacacttgatgtactgccaagtgggcagttt |
| | accgtaaatactccacccattgacgtcaatggaaagtccctattggcgttactatgggaacatacgt |
| | cattattgacgtcaatgggcgggggtcgttgggcggtcagccaggcgggccatttaccgtaagttat |
| | gtaacgGGCCTGCTGCCGGCTCTGCGGCCTCTTCCGCGTCTTCGCCTTCGCCCTCAGACGAGTCGGA |
| | TCTCCCTTTGGGCCGCCTCCCCGCCTGTCTAGCTTGACTGACTGAGATACAGCGTACCTTCAGCTCA |
| | CAGACATGATAAGATACATTGATGAGTTTGGACAAACCACAACTAGAATGCAGTGAAAAAAATGCTT |
| | TATTTGTGAAATTTGTGATGCTATTGCTTTATTTGTAACCATTATAAGCTGCAATAAACAAGTTAAC |
| | AACAACAATTGCATTCATTTTATGTTTCAGGTTCAGGGGGAGGTGTGGGAGGTTTTTTAAAGCAAGT |
| | AAAACCTCTACAAATGTGGTATTGGCCCATCTCTATCGGTATCGTAGCATAACCCCTTGGGGCCTCT |
| | AAACGGGTCTTGAGGGGTTTTTTGTGCCCCTCGGGCCGGATTGCTATCTACCGGCATTGGCGCAGAA |
| | AAAAATGCCTGATGCGACGCTGCGCGTCTTATACTCCCACATATGCCAGATTCAGCAACGGATACGG |
| | CTTCCCCAACTTGCCCACTTCCATACGTGTCCTCCTTACCAGAAATTTATCCTTAAGGTCGTCAGCT |
| | ATCCTGCAGGCGATCTCTCGATTTCGATCAAGACATTCCTTTAATGGTCTTTTCTGGACACCACTAG |
| | GGGTCAGAAGTAGTTCATCAAACTTTCTTCCCTCCCTAATCTCATTGGTTACCTTGGGCTATCGAAA |
| | CTTAATTAAGCGATCTGCATCTCAATTAGTCAGCAACCATAGTCCCGCCCCTAACTCCGCCCATCCC |
| | GCCCCTAACTCCGCCCAGTTCCGCCCATTCTCCGCCCCATCGCTGACTAATTTTTTTTATTTATGCA |
| | GAGGCCGAGGCCGCCTCGGCCTCTGAGCTATTCCAGAAGTAGTGAGGAGGCTTTTTTGGAGGCCTAG |
| | GCTTTTGCAAAGGAGGTAGCCAACATGATTGAACAAGATGGATTGCACGCAGGTTCTCCCGCCGCTT |
| | GGGTGGAGAGGCTATTCGGCTATGACTGGGCACAACAGACAATCGGCTGCTCTGATGCCGCCGTGTT |
| | CCGGCTGTCAGCGCAGGGGCGCCCGGTTCTTTTTGTCAAGACCGACCTGTCCGGTGCCCTGAATGAA |
| | CTCCAGGACGAGGCAGCGCGGCTATCGTGGCTGGCCACGACGGGCGTTCCTTGCGCAGCTGTGCTCG |
| | ACGTTGTCACTGAAGCGGGAAGGGACTGGCTGCTATTGGGCGAAGTGCCGGGGCAGGATCTCCTGTC |
| | ATCTCACCTTGCTCCTGCCGAGAAAGTATCCATCATGGCTGATGCAATGCGGCGGCTGCATACGCTT |
| | GATCCGGCTACCTGCCCATTCGACCACCAAGCGAAACATCGCATCGAGCGAGCACGTACTCGGATGG |
| | AAGCCGGTCTTGTCGATCAGGATGATCTGGACGAAGAGCATCAGGGGCTCGCGCCAGCCGAACTGTT |
| | CGCCAGGCTCAAGGCGCGGATGCCCGACGGCGAGGATCTCGTCGTGACCCACGGCGATGCCTGCTTG |
| | CCGAATATCATGGTGGAAAATGGCCGCTTTTCTGGATTCATCGACTGTGGCCGGCTGGGTGTGGCGG |
| | ACCGCTATCAGGACATAGCGTTGGCTACCCGTGATATTGCTGAAGAGCTTGGCGGCGAATGGGCTGA |
| | CCGCTTCCTCGTGCTTTACGGTATCGCCGCTCCCGATTCGCAGCGCATCGCCTTCTATCGCCTTCTT |
| | GACGAGTTCTTCTAGTATGTAAGCCCTGTGCCTTCTAGTTGCCAGCCATCTGTTGTTTGCCCCTCCC |
| | CCGTGCCTTCCTTGACCCTGGAAGGTGCCACTCCCACTGTCCTTTCCTAATAAAATGAGGAAATTGC |
| | ATCGCATTGTCTGAGTAGGTGTCATTCTATTCTGGGGGGTGGGGTGGGGCAGGACAGCAAGGGGGAG |
| | GATTGGGAAGACAATAGCAGGCATGCTGGGGATGCGGTGGGCTCTATGGTTAATTAACCAGTCAAGT |
| | CAGCTACTTGGCGAGATCGACTTGTCTGGGTTTCGACTACGCTCAGAATTGCGTCAGTCAAGTTCGA |
| | TCTGGTCCTTGCTATTGCACCCGTTCTCCGATTACGAGTTTCATTTAAATCATGTGAGCAAAAGGCC |
| | AGCAAAAGGCCAGGAACCGTAAAAAGGCCGCGTTGCTGGCGTTTTTCCATAGGCTCCGCCCCCCTGA |
| | CGAGCATCACAAAAATCGACGCTCAAGTCAGAGGTGGCGAAACCCGACAGGACTATAAAGATACCAG |
| | GCGTTTCCCCCTGGAAGCTCCCTCGTGCGCTCTCCTGTTCCGACCCTGCCGCTTACCGGATACCTGT |
| | CCGCCTTTCTCCCTTCGGGAAGCGTGGCGCTTTCTCATAGCTCACGCTGTAGGTATCTCAGTTCGGT |
| | GTAGGTCGTTCGCTCCAAGCTGGGCTGTGTGCACGAACCCCCCGTTCAGCCCGACCGCTGCGCCTTA |
| | TCCGGTAACTATCGTCTTGAGTCCAACCCGGTAAGACACGACTTATCGCCACTGGCAGCAGCCACTG |
| | GTAACAGGATTAGCAGAGCGAGGTATGTAGGCGGTGCTACAGAGTTCTTGAAGTGGTGGCCTAACTA |
| | CGGCTACACTAGAAGAACAGTATTTGGTATCTGCGCTCTGCTGAAGCCAGTTACCTTCGGAAAAAGA |
| | GTTGGTAGCTCTTGATCCGGCAAACAAACCACCGCTGGTAGCGGTGGTTTTTTTGTTTGCAAGCAGC |
| | AGATTACGCGCAGAAAAAAAGGATCTCAAGAAGATCCTTTGATCTTTTCTACGGGGTCTGACGCTCA |
| | GTGGAACGAAAACTCACGTTAAGGGATTTTGGTCATGAGATTATCAAAAAGGATCTTCACCTAGATC |
| | CTTTTAAATTAAAAATGAAGTTTTAAATCAATCTAAAGTATATATGAGTAAACTTGGTCTGACAGTT |
| | ACCAATGCTTAATCAGTGAGGCACCTATCTCAGCGATCTGTCTATTTCGTTCATCCATAGTTGCATT |
| | TAAATTTCCGAACTCTCCAAGGCCCTCGTCGGAAAATCTTCAAACCTTTCGTCCGATCCATCTTGCA |
| | GGCTACCTCTCGAACGAACTATCGCAAGTCTCTTGGCCGGCCTTGCGCCTTGGCTATTGCTTGGCAG |
| | CGCCTATCGCCAGGTATTACTCCAATCCCGAATATCCGAGATCGGGATCACCCGAGAGAAGTTCAAC |
| | CTACATCCTCAATCCCGATCTATCCGAGATCCGAGGAATATCGAAATCGGGGCGCGCCTGGTGTACC |
| | GAGAACGATCCTCTCAGTGCGAGTC (SEQ ID NO: 105) |
| ret-016- | ACCGAGAACGATCCTCTCAGTGCGAGTCTCGACGATCCATATCGTTGCTTGGCAGTCAGCCAGTCGG |
| line1- | AATCCAGCTTGGGACCCAGGAAGTCCAATCGTCAGATATTGTACTCAAGCCTGGTCACGGCAGCGTA |
| gfp- | CCGATCTGTTTAAACCTAGATATTGATAGTCTGATCGGTCAACGTATAATCGAGTCCTAGCTTTTGC |
| orf2- | AAACATCTATCAAGAGACAGGATCAGCAGGAGGCTTTCGCATGAGTATTCAACATTTCCGTGTCGCC |
| c_nucleo- | CTTATTCCCTTTTTTGCGGCATTTTGCCTTCCTGTTTTTGCTCACCCAGAAACGCTGGTGAAAGTAA |
| plasmin- | AAGATGCTGAAGATCAGTTGGGTGCGCGAGTGGGTTACATCGAACTGGATCTCAACAGCGGTAAGAT |
| nls | CCTTGAGAGTTTTCGCCCCGAAGAACGCTTTCCAATGATGAGCACTTTTAAAGTTCTGCTATGTGGC |
| | GCGGTATTATCCCGTATTGACGCCGGGCAAGAGCAACTCGGTCGCCGCATACACTATTCTCAGAATG |
| | ACTTGGTTGAGTATTCACCAGTCACAGAAAAGCATCTTACGGATGGCATGACAGTAAGAGAATTATG |
| | CAGTGCTGCCATAACCATGAGTGATAACACTGCGGCCAACTTACTTCTGACAACGATTGGAGGACCG |
| | AAGGAGCTAACCGCTTTTTTGCACAACATGGGGGATCATGTAACTCGCCTTGATCGTTGGGAACCGG |
| | AGCTGAATGAAGCCATACCAAACGACGAGCGTGACACCACGATGCCTGTAGCAATGGCAACAACCTT |
| | GCGTAAACTATTAACTGGCGAACTACTTACTCTAGCTTCCCGGCAACAGTTGATAGACTGGATGGAG |
| | GCGGATAAAGTTGCAGGACCACTTCTGCGCTCGGCCCTTCCGGCTGGCTGGTTTATTGCTGATAAAT |
| | CTGGAGCCGGTGAGCGTGGGTCTCGCGGTATCATTGCAGCACTGGGGCCAGATGGTAAGCCCTCCCG |
| | TATCGTAGTTATCTACACGACGGGGAGTCAGGCAACTATGGATGAACGAAATAGACAGATCGCTGAG |
| | ATAGGTGCCTCACTGATTAAGCATTGGTAACCGATTCTAGGTGCATTGGCGCAGAAAAAAATGCCTG |
| | ATGCGACGCTGCGCGTCTTATACTCCCACATATGCCAGATTCAGCAACGGATACGGCTTCCCCAACT |
| | TGCCCACTTCCATACGTGTCCTCCTTACCAGAAATTTATCCTTAAGATCGTTTAAACTCGACTCTGG |
| | CTCTATCGAATCTCCGTCGTTTCGAGCTTACGCGAACAGCCGTGGCGCTCATTTGCTCGTCGGGCAT |
| | CGAATCTCGTCAGCTATCGTCAGCTTACCTTTTTGGCAGCGATCGCGGCTCCCGACATCTTGGACCA |
| | TTAGCTCCACAGGTATCTTCTTCCCTCTAGTGGTCATAACAGCAGCTTCAGCTACCTCTCAATTCAA |
| | AAAACCCCTCAAGACCCGTTTAGAGGCCCCAAGGGGTTATGCTATCAATCGTTGCGTTACACACACA |
| | AAAAACCAACACACATCCATCTTCGATGGATAGCGATTTTATTATCTAACTGCTGATCGAGTGTAGC |
| | CAGATCTAGTAATCAATTACGGGGTCATTAGTTCATAGCCCATATATGGAGTTCCGCGTTACATAAC |
| | TTACGGTAAATGGCCCGCCTGGCTGACCGCCCAACGACCCCCGCCCATTGACGTCAATAATGACGTA |
| | TGTTCCCATAGTAACGCCAATAGGGACTTTCCATTGACGTCAATGGGTGGAGTATTTACGGTAAACT |
| | GCCCACTTGGCAGTACATCAAGTGTATCATATGCCAAGTACGCCCCCTATTGACGTCAATGACGGTA |
| | AATGGCCCGCCTGGCATTATGCCCAGTACATGACCTTATGGGACTTTCCTACTTGGCAGTACATCTA |
| | CGTATTAGTCATCGCTATTACCATGCTGATGCGGTTTTGGCAGTACATCAATGGGCGTGGATAGCGG |
| | TTTGACTCACGGGGATTTCCAAGTCTCCACCCCATTGACGTCAATGGGAGTTTGTTTTGGCACCAAA |
| | ATCAACGGGACTTTCCAAAATGTCGTAACAACTCCGCCCCATTGACGCAAATGGGCGGTAGGCGTGT |
| | ACGGTGGGAGGTCTATATAAGCAGAGCTGGTTTAGTGAACCGTCAGATCAGATCTTTGTCGATCCTA |
| | CCATCCACTCGACACACCCGCCAGCGGCCGCTAATACGACTCACTATAGGGAGAAGTACTGCCACCA |
| | TGGGCAAGAAGCAAAATCGCAAGACGGGGAATTCCAAGACACAATCCGCTAGCCCACCACCTAAAGA |
| | GCGTTCTAGCTCCCCTGCTACTGAGCAGTCCTGGATGGAAAACGACTTCGATGAACTCCGGGAAGAG |
| | GGATTTAGGCGATCCAACTATTCAGAACTCCGCGAAGATATCCAGACAAAGGGGAAGGAAGTCGAGA |
| | ATTTCGAGAAGAACCTCGAGGAGTGCATCACCCGTATCACAAACACTGAGAAATGTCTCAAAGAACT |
| | CATGGAACTTAAGACAAAAGCCAGGGAGCTTCGAGAGGAGTGTCGGAGTCTGAGATCCAGGTGTGAC |
| | CAGCTCGAGGAGCGCGTGAGCGCGATGGAAGACGAGATGAACGAGATGAAAAGAGAGGGCAAATTCA |
| | GGGAGAAGCGCATTAAGAGGAACGAACAGAGTCTGCAGGAGATTTGGGATTACGTCAAGAGGCCTAA |
| | CCTGCGGTTGATCGGCGTCCCCGAGAGCGACGTAGAAAACGGGACTAAACTGGAGAATACACTTCAA |
| | GACATCATTCAAGAAAATTTTCCAAACCTGGCTCGGCAAGCTAATGTGCAAATCCAAGAGATCCAAC |
| | GCACACCCCAGCGGTATAGCTCTCGGCGTGCCACCCCTAGGCATATTATCGTGCGCTTTACTAAGGT |
| | GGAGATGAAAGAGAAGATGCTGCGAGCCGCTCGGGAAAAGGGAAGGGTGACTTTGAAGGGCAAACCT |
| | ATTCGGCTGACGGTTGACCTTAGCGCCGAGACACTCCAGGCACGCCGGGAATGGGGCCCCATCTTTA |
| | ATATCCTGAAGGAGAAGAACTTCCAGCCACGAATCTCTTACCCTGCAAAGTTGAGTTTTATCTCCGA |
| | GGGTGAGATTAAGTATTTCATCGATAAACAGATGCTGCGAGACTTCGTGACAACTCGCCCAGCTCTC |
| | AAGGAACTGCTCAAAGAGGCTCTTAATATGGAGCGCAATAATAGATATCAACCCTTGCAGAACCACG |
| | CAAAGATGTGAGACAGCCGTCAGACCATCAAGACTAGGAAGAAACTGCATCAACTAATGAGCAAAAT |
| | CACCAGCTAACATCATAGTATACATGACCGGCTCTAACTCACATATCACCATCCTTACACTTAACAT |
| | TAACGGCCTCAACTCAGCTATCAAGCGCCATCGGCTGGCCAGCTGGATCAAATCACAGGATCCAAGC |
| | GTTTGTTGCATCCAAGAGACCCACCTGACCTGTAGAGATACTCACCGCCTCAAGATCAAGGGATGGC |
| | GAAAGATTTATCAGGCGAACGGTAAGCAGAAGAAAGCCGGAGTCGCAATTCTGGTCTCAGACAAGAC |
| | GGATTTCAAGCCCACCAAAATTAAGCGTGATAAGGAAGGTCACTATATTATGGTGAAAGGCAGCATA |
| | CAGCAGGAAGAACTTACCATATTGAACATCTACGCGCCAAACACCGGCGCACCTCGCTTTATCAAAC |
| | AGGTCCTGTCCGATCTGCAGCGAGATCTGGATTCTCATACGTTGATTATGGGTGATTTCAATACACC |
| | ATTGAGCACCCTGGATCGCAGCACCAGGCAAAAGGTAAATAAAGACACGCAAGAGCTCAATAGCGCA |
| | CTGCATCAGGCAGATCTCATTGATATTTATCGCACTCTTCATCCTAAGAGTACCGAGTACACATTCT |
| | TCAGCGCCCCACATCATACATACTCAAAGATCGATCATATCGTCGGCTCAAAGGCTCTGCTGTCAAA |
| | GTGCAAGCGCACAGAGATAATTACAAATTACCTGTCAGATCATAGCGCGATCAAGCTCGAGCTGAGA |
| | ATCAAGAACCTGACCCAGAGCCGGAGTACCACTTGGAAGCTTAATAACCTGCTGCTCAACGATTATT |
| | GGGTCCACAATGAGATGAAGGCAGAGATTAAAATGTTCTTCGAAACAAATGAGAATAAGGATACTAC |
| | CTATCAAAACCTTTGGGATGCCTTTAAGGCCGTCTGCAGAGGCAAGTTCATCGCCCTCAACGCCTAT |
| | AAAAGAAAACAAGAGAGATCTAAGATCGATACTCTCACCTCTCAGCTGAAGGAGTTGGAGAAACAGG |
| | AACAGACCCACTCCAAGGCGTCAAGACGGCAGGAGATCACAAAGATTCGCGCCGAGTTGAAAGAGAT |
| | CGAAACCCAAAAGACTCTTCAGAAAATTAACGAGTCTCGTAGTTGGTTCTTCGAGCGGATTAATAAG |
| | ATAGACAGACCTCTGGCACGACTGATTAAGAAGAAGCGCGAAAAGAACCAGATTGATACCATCAAGA |
| | ACGACAAGGGCGACATCACTACTGACCCGACCGAGATCCAGACCACTATTCGGGAGTATTATAAGCA |
| | TTTGTATGCTAACAAGCTTGAGAACCTGGAAGAGATGGACACTTTTCTGGATACCTATACTCTGCCA |
| | CGGCTTAATCAAGAGGAAGTCGAGTCCCTCAACCGCCCAATTACAGGAAGCGAGATTGTGGCCATAA |
| | TTAACTCCCTGCCGACAAAGAAATCTCCTGGTCCGGACGGGTTTACAGCTGAGTTTTATCAACGGTA |
| | TATGGAAGAGCTTGTACCGTTTCTGCTCAAGCTCTTTCAGTCTATAGAAAAGGAAGGCATCTTGCCC |
| | AATTCCTTCTACGAAGCTTCTATAATACTTATTCCCAAACCAGGACGCGATACCACAAAGAAGGAAA |
| | ACTTCCGGCCCATTAGTCTCATGAATATCGACGCTAAAATATTGAACAAGATTCTCGCCAACAGAAT |
| | CCAACAACATATTAAGAAATTGATACATCACGACCAGGTGGGGTTTATACCTGGCATGCAGGGCTGG |
| | TTTAACATCCGGAAGAGTATTAACGTCATTCAACACATTAATAGAGCTAAGGATAAGAATCATATGA |
| | TCATCTCTATAGACGCGGAAAAGGCATTCGATAAGATTCAGCAGCCATTTATGCTCAAGACTCTGAA |
| | CAAACTCGGCATCGACGGAACATATTTTAAGATTATTCGCGCAATTTACGATAAGCCGACTGCTAAC |
| | ATTATCCTTAACGGCCAAAAGCTCGAGGCCTTTCCGCTCAAGACTGGAACCCGCCAAGGCTGTCCCC |
| | TCTCCCCGCTTTTGTTTAATATTGTACTCGAGGTGCTGGCTAGGGCTATTCGTCAAGAGAAAGAGAT |
| | TAAAGGGATACAGCTCGGGAAGGAAGAGGTCAAGCTTTCCTTGTTCGCCGATGATATGATTGTGTAC |
| | CTGGAGAATCCTATTGTGTCTGCTCAGAACCTTCTTAAACTTATTTCTAACTTTAGCAAGGTCAGCG |
| | GCTATAAGATTAACGTCCAGAAATCTCAGGCCTTTCTGTACACAAATAATCGACAGACCGAATCCCA |
| | GATAATGGGTGAGCTTCCGTTTGTCATAGCCAGCAAAAGGATAAAGTATCTCGGAATCCAGCTGACA |
| | CGAGACGTTAAAGATTTGTTTAAGGAAAATTACAAGCCTCTCCTGAAAGAGATTAAGGAAGATACTA |
| | ATAAGTGGAAGAATATCCCCTGTTCATGGGTTGGCAGAATCAACATAGTGAAGATGGCAATACTTCC |
| | TAAAGTGATATATCGCTTTAACGCCATCCCAATTAAACTGCCTATGACCTTCTTTACGGAGCTCGAG |
| | AAAACAACCCTTAAATTTATATGGAATCAAAAGAGAGCAAGAATAGCGAAGTCCATCTTGAGCCAGA |
| | AGAATAAGGCCGGTGGGATTACTTTGCCTGATTTTAAGTTGTATTATAAAGCCACAGTAACTAAGAC |
| | AGCCTGGTATTGGTATCAGAATAGAGACATCGACCAGTGGAATCGGACCGAACCATCAGAGATAATG |
| | CCCCACATCTATAATTACCTTATATTCGATAAGCCAGAAAAGAATAAACAGTGGGGCAAAGACAGCC |
| | TCTTCAACAAGTGGTGTTGGGAGAATTGGCTGGCCATATGCCGGAAACTCAAGCTCGACCCCTTTCT |
| | TACACCCTACACTAAAATCAACAGTAGGTGGATCAAGGACTTGAATGTCAAGCCAAAGACTATAAAG |
| | ACACTGGAAGAGAATCTTGGGATCACAATACAAGATATAGGCGTCGGCAAAGATTTTATGTCAAAGA |
| | CGCCCAAGGCCATGGCCACTAAGGATAAGATTGATAAGTGGGACCTTATTAAGCTCAAAAGCTTCTG |
| | TACTGCCAAGGAGACCACGATCAGAGTTAATAGGCAGCCCACTACATGGGAAAAGATTTTCGCCACT |
| | TATTCATCAGATAAGGGGTTGATAAGCAGAATATATAACGAGCTGAAGCAGATCTACAAGAAGAAAA |
| | CGAATAATCCCATCAAGAAGTGGGCAAAAGATATGAACAGGCATTTTAGCAAAGAGGATATCTACGC |
| | CGCGAAGAAGCATATGAAGAAGTGTAGTTCAAGCTTGGCCATTCGTGAGATGCAGATTAAGACGACC |
| | ATGCGATACCACCTTACCCCAGTGAGGATGGCAATTATCAAGAAATCTGGCAATAATAGATGTTGGC |
| | GGGGCTGTGGCGAGATTGGCACCCTGCTCCATTGCTGGTGGGATTGCAAGCTGGTGCAGCCGCTTTG |
| | GAAATCAGTCTGGCGCTTTCTGAGGGACCTCGAGCTTGAGATTCCCTTCGATCCCGCAATTCCCTTG |
| | CTCGGAATCTATCCTAACGAATACAAGAGCTGTTGTTACAAGGATACGTGTACCCGGATGTTCATCG |
| | CGGCCTTGTTTACGATAGCTAAGACGTGGAATCAGCCTAAGTGCCCCACAATGATCGATTGGATCAA |
| | GAAAATGTGGCATATTTATACCATGGAGTATTACGCAGCAATTAAGAATGACGAATTTATTTCCTTC |
| | GTTGGGACCTGGATGAAGCTGGAGACTATTATTCTGAGCAAGCTGTCTCAGGAGCAAAAGACAAAGC |
| | ATAGAATCTTCTCTCTCATTGGTGGTAACGACTACAAAGACGATGACGACAAGaaaaggccggcggc |
| | cacgaaaaaggccggccaggcaaaaaagaaaaagTAAAGCGCTTCTAGAAGTTGTCTCCTCCTGCAC |
| | TGACTGACTGATACAATCGATTTCTGGATCCGCAGGCCTAATCAACCTCTGGATTACAAAATTTGTG |
| | AAAGATTGACTGGTATTCTTAACTATGTTGCTCCTTTTACGCTATGTGGATACGCTGCTTTAATGCC |
| | TTTGTATCATGCTATTGCTTCCCGTATGGCTTTCATTTTCTCCTCCTTGTATAAATCCTGGTTGCTG |
| | TCTCTTTATGAGGAGTTGTGGCCCGTTGTCAGGCAACGTGGCGTGGTGTGCACTGTGTTTGCTGACG |
| | CAACCCCCACTGGTTGGGGCATTGCCACCACCTGTCAGCTCCTTTCCGGGACTTTCGCTTTCCCCCT |
| | CCCTATTGCCACGGCGGAACTCATCGCCGCCTGCCTTGCCCGCTGCTGGACAGGGGCTCGGCTGTTG |
| | GGCACTGACAATTCCGTGGTGTTGTCGGGGAAGCTGACGTCCTTTCCATGGCTGCTCGCCTGTGTTG |
| | CCACCTGGATTCTGCGCGGGACGTCCTTCTGCTACGTCCCTTCGGCCCTCAATCCAGCGGACCTTCC |
| | TTCCCGCtgagagacacaaaaaattccaacacactattgcaatgaaaataaatttcctttattagcc |
| | agaagtcagatgctcaaggggcttcatgatgtccccataatttttggcagagggaaaaagatctcag |
| | tggtatttgtgagccagggcattggccttctgataggcagcctgcacctgaggagtgcggccgcttt |
| | acttgtacagctcgtccatgccgagagtgatcccggcggcggtcacgaactccagcaggaccatgtg |
| | atcgcgcttctcgttggggtctttgctcagggcggactgggtgctcaggtagtggttgtcgggcagc |
| | agcacggggccgtcgccgatgggggtgttctgctggtagtggtcggcgagctgcacgctgccgtcct |
| | cgatgttgtggcggatcttgaagttcaccttgatgccgttcttctgcttgtcggccatgatatagac |
| | gttgtggctgttgtagttgtactccagcttgtgccccaggatgttgccgtcctccttgaagtcgatg |
| | cccttcagctcgatgcggttcaccagggtgtcgccctcgaacttcacctcggcgcgggtcttgtagt |
| | tgccgtcgtccttgaagaagatggtgcgctcctggacgtagccttcgggcatggcggacttgaagaa |
| | gtcgtgctgcttcatgtggtcggggtagcggctgaagcactgcacgccgtaggtcagggtggtcacg |
| | agggtgggccagggcacgggcagcttgccggtggtgcagatgaacttcagggtcagcttgccgtagg |
| | tggcatcgccctcgccctcgccggacacgctgaacttgtggccgtttacgtcgccgtccagctcgac |
| | caggatgggcaccaccccggtgaacagctcctcgcccttgctcaccatggtggcgggatctgacggt |
| | tcactaaaccagctctgcttatatagacctcccaccgtacacgcctaccgcccatttgcgtcaatgg |
| | ggcggagttgttacgacattttggaaagtcccgttgattttggtgccaaaacaaactcccattgacg |
| | tcaatggggtggagacttggaaatccccgtgagtcaaaccgctatccacgcccattgatgtactgcc |
| | aaaaccgcatcaccatggtaatagcgatgactaatacgtagatgtactgccaagtaggaaagtccca |
| | taaggtcatgtactgggcataatgccaggcgggccatttaccgtcattgacgtcaatagggggcgta |
| | cttggcatatgatacacttgatgtactgccaagtgggcagtttaccgtaaatactccacccattgac |
| | gtcaatggaaagtccctattggcgttactatgggaacatacgtcattattgacgtcaatgggcgggg |
| | gtcgttgggcggtcagccaggcgggccatttaccgtaagttatgtaacgGGCCTGCTGCCGGCTCTG |
| | CGGCCTCTTCCGCGTCTTCGCCTTCGCCCTCAGACGAGTCGGATCTCCCTTTGGGCCGCCTCCCCGC |
| | CTGTCTAGCTTGACTGACTGAGATACAGCGTACCTTCAGCTCACAGACATGATAAGATACATTGATG |
| | AGTTTGGACAAACCACAACTAGAATGCAGTGAAAAAAATGCTTTATTTGTGAAATTTGTGATGCTAT |
| | TGCTTTATTTGTAACCATTATAAGCTGCAATAAACAAGTTAACAACAACAATTGCATTCATTTTATG |
| | TTTCAGGTTCAGGGGGAGGTGTGGGAGGTTTTTTAAAGCAAGTAAAACCTCTACAAATGTGGTATTG |
| | GCCCATCTCTATCGGTATCGTAGCATAACCCCTTGGGGCCTCTAAACGGGTCTTGAGGGGTTTTTTG |
| | TGCCCCTCGGGCCGGATTGCTATCTACCGGCATTGGCGCAGAAAAAAATGCCTGATGCGACGCTGCG |
| | CGTCTTATACTCCCACATATGCCAGATTCAGCAACGGATACGGCTTCCCCAACTTGCCCACTTCCAT |
| | ACGTGTCCTCCTTACCAGAAATTTATCCTTAAGGTCGTCAGCTATCCTGCAGGCGATCTCTCGATTT |
| | CGATCAAGACATTCCTTTAATGGTCTTTTCTGGACACCACTAGGGGTCAGAAGTAGTTCATCAAACT |
| | TTCTTCCCTCCCTAATCTCATTGGTTACCTTGGGCTATCGAAACTTAATTAAGCGATCTGCATCTCA |
| | ATTAGTCAGCAACCATAGTCCCGCCCCTAACTCCGCCCATCCCGCCCCTAACTCCGCCCAGTTCCGC |
| | CCATTCTCCGCCCCATCGCTGACTAATTTTTTTTATTTATGCAGAGGCCGAGGCCGCCTCGGCCTCT |
| | GAGCTATTCCAGAAGTAGTGAGGAGGCTTTTTTGGAGGCCTAGGCTTTTGCAAAGGAGGTAGCCAAC |
| | ATGATTGAACAAGATGGATTGCACGCAGGTTCTCCCGCCGCTTGGGTGGAGAGGCTATTCGGCTATG |
| | ACTGGGCACAACAGACAATCGGCTGCTCTGATGCCGCCGTGTTCCGGCTGTCAGCGCAGGGGCGCCC |
| | GGTTCTTTTTGTCAAGACCGACCTGTCCGGTGCCCTGAATGAACTCCAGGACGAGGCAGCGCGGCTA |
| | TCGTGGCTGGCCACGACGGGCGTTCCTTGCGCAGCTGTGCTCGACGTTGTCACTGAAGCGGGAAGGG |
| | ACTGGCTGCTATTGGGCGAAGTGCCGGGGCAGGATCTCCTGTCATCTCACCTTGCTCCTGCCGAGAA |
| | AGTATCCATCATGGCTGATGCAATGCGGCGGCTGCATACGCTTGATCCGGCTACCTGCCCATTCGAC |
| | CACCAAGCGAAACATCGCATCGAGCGAGCACGTACTCGGATGGAAGCCGGTCTTGTCGATCAGGATG |
| | ATCTGGACGAAGAGCATCAGGGGCTCGCGCCAGCCGAACTGTTCGCCAGGCTCAAGGCGCGGATGCC |
| | CGACGGCGAGGATCTCGTCGTGACCCACGGCGATGCCTGCTTGCCGAATATCATGGTGGAAAATGGC |
| | CGCTTTTCTGGATTCATCGACTGTGGCCGGCTGGGTGTGGCGGACCGCTATCAGGACATAGCGTTGG |
| | CTACCCGTGATATTGCTGAAGAGCTTGGCGGCGAATGGGCTGACCGCTTCCTCGTGCTTTACGGTAT |
| | CGCCGCTCCCGATTCGCAGCGCATCGCCTTCTATCGCCTTCTTGACGAGTTCTTCTAGTATGTAAGC |
| | CCTGTGCCTTCTAGTTGCCAGCCATCTGTTGTTTGCCCCTCCCCCGTGCCTTCCTTGACCCTGGAAG |
| | GTGCCACTCCCACTGTCCTTTCCTAATAAAATGAGGAAATTGCATCGCATTGTCTGAGTAGGTGTCA |
| | TTCTATTCTGGGGGGTGGGGTGGGGCAGGACAGCAAGGGGGAGGATTGGGAAGACAATAGCAGGCAT |
| | GCTGGGGATGCGGTGGGCTCTATGGTTAATTAACCAGTCAAGTCAGCTACTTGGCGAGATCGACTTG |
| | TCTGGGTTTCGACTACGCTCAGAATTGCGTCAGTCAAGTTCGATCTGGTCCTTGCTATTGCACCCGT |
| | TCTCCGATTACGAGTTTCATTTAAATCATGTGAGCAAAAGGCCAGCAAAAGGCCAGGAACCGTAAAA |
| | AGGCCGCGTTGCTGGCGTTTTTCCATAGGCTCCGCCCCCCTGACGAGCATCACAAAAATCGACGCTC |
| | AAGTCAGAGGTGGCGAAACCCGACAGGACTATAAAGATACCAGGCGTTTCCCCCTGGAAGCTCCCTC |
| | GTGCGCTCTCCTGTTCCGACCCTGCCGCTTACCGGATACCTGTCCGCCTTTCTCCCTTCGGGAAGCG |
| | TGGCGCTTTCTCATAGCTCACGCTGTAGGTATCTCAGTTCGGTGTAGGTCGTTCGCTCCAAGCTGGG |
| | CTGTGTGCACGAACCCCCCGTTCAGCCCGACCGCTGCGCCTTATCCGGTAACTATCGTCTTGAGTCC |
| | AACCCGGTAAGACACGACTTATCGCCACTGGCAGCAGCCACTGGTAACAGGATTAGCAGAGCGAGGT |
| | ATGTAGGCGGTGCTACAGAGTTCTTGAAGTGGTGGCCTAACTACGGCTACACTAGAAGAACAGTATT |
| | TGGTATCTGCGCTCTGCTGAAGCCAGTTACCTTCGGAAAAAGAGTTGGTAGCTCTTGATCCGGCAAA |
| | CAAACCACCGCTGGTAGCGGTGGTTTTTTTGTTTGCAAGCAGCAGATTACGCGCAGAAAAAAAGGAT |
| | CTCAAGAAGATCCTTTGATCTTTTCTACGGGGTCTGACGCTCAGTGGAACGAAAACTCACGTTAAGG |
| | GATTTTGGTCATGAGATTATCAAAAAGGATCTTCACCTAGATCCTTTTAAATTAAAAATGAAGTTTT |
| | AAATCAATCTAAAGTATATATGAGTAAACTTGGTCTGACAGTTACCAATGCTTAATCAGTGAGGCAC |
| | CTATCTCAGCGATCTGTCTATTTCGTTCATCCATAGTTGCATTTAAATTTCCGAACTCTCCAAGGCC |
| | CTCGTCGGAAAATCTTCAAACCTTTCGTCCGATCCATCTTGCAGGCTACCTCTCGAACGAACTATCG |
| | CAAGTCTCTTGGCCGGCCTTGCGCCTTGGCTATTGCTTGGCAGCGCCTATCGCCAGGTATTACTCCA |
| | ATCCCGAATATCCGAGATCGGGATCACCCGAGAGAAGTTCAACCTACATCCTCAATCCCGATCTATC |
| | CGAGATCCGAGGAATATCGAAATCGGGGCGCGCCTGGTGT (SEQ ID NO: 106) |
| RET- | CCGAGAACGATCCTCTCAGTGCGAGTCTCGACGATCCATATCGTTGCTTGGCAGTCAGCCAGTCGGA |
| 017 | ATCCAGCTTGGGACCCAGGAAGTCCAATCGTCAGATATTGTACTCAAGCCTGGTCACGGCAGCGTAC |
| LINE1- | CGATCTGTTTAAACCTAGATATTGATAGTCTGATCGGTCAACGTATAATCGAGTCCTAGCTTTTGCA |
| GFP | AACATCTATCAAGAGACAGGATCAGCAGGAGGCTTTCGCATGAGTATTCAACATTTCCGTGTCGCCC |
| ORF2- | TTATTCCCTTTTTTGCGGCATTTTGCCTTCCTGTTTTTGCTCACCCAGAAACGCTGGTGAAAGTAAA |
| C_Linker_ | AGATGCTGAAGATCAGTTGGGTGCGCGAGTGGGTTACATCGAACTGGATCTCAACAGCGGTAAGATC |
| Nucleo- | CTTGAGAGTTTTCGCCCCGAAGAACGCTTTCCAATGATGAGCACTTTTAAAGTTCTGCTATGTGGCG |
| plasmin- | CGGTATTATCCCGTATTGACGCCGGGCAAGAGCAACTCGGTCGCCGCATACACTATTCTCAGAATGA |
| NLS | CTTGGTTGAGTATTCACCAGTCACAGAAAAGCATCTTACGGATGGCATGACAGTAAGAGAATTATGC |
| | AGTGCTGCCATAACCATGAGTGATAACACTGCGGCCAACTTACTTCTGACAACGATTGGAGGACCGA |
| | AGGAGCTAACCGCTTTTTTGCACAACATGGGGGATCATGTAACTCGCCTTGATCGTTGGGAACCGGA |
| | GCTGAATGAAGCCATACCAAACGACGAGCGTGACACCACGATGCCTGTAGCAATGGCAACAACCTTG |
| | CGTAAACTATTAACTGGCGAACTACTTACTCTAGCTTCCCGGCAACAGTTGATAGACTGGATGGAGG |
| | CGGATAAAGTTGCAGGACCACTTCTGCGCTCGGCCCTTCCGGCTGGCTGGTTTATTGCTGATAAATC |
| | TGGAGCCGGTGAGCGTGGGTCTCGCGGTATCATTGCAGCACTGGGGCCAGATGGTAAGCCCTCCCGT |
| | ATCGTAGTTATCTACACGACGGGGAGTCAGGCAACTATGGATGAACGAAATAGACAGATCGCTGAGA |
| | TAGGTGCCTCACTGATTAAGCATTGGTAACCGATTCTAGGTGCATTGGCGCAGAAAAAAATGCCTGA |
| | TGCGACGCTGCGCGTCTTATACTCCCACATATGCCAGATTCAGCAACGGATACGGCTTCCCCAACTT |
| | GCCCACTTCCATACGTGTCCTCCTTACCAGAAATTTATCCTTAAGATCGTTTAAACTCGACTCTGGC |
| | TCTATCGAATCTCCGTCGTTTCGAGCTTACGCGAACAGCCGTGGCGCTCATTTGCTCGTCGGGCATC |
| | GAATCTCGTCAGCTATCGTCAGCTTACCTTTTTGGCAGCGATCGCGGCTCCCGACATCTTGGACCAT |
| | TAGCTCCACAGGTATCTTCTTCCCTCTAGTGGTCATAACAGCAGCTTCAGCTACCTCTCAATTCAAA |
| | AAACCCCTCAAGACCCGTTTAGAGGCCCCAAGGGGTTATGCTATCAATCGTTGCGTTACACACACAA |
| | AAAACCAACACACATCCATCTTCGATGGATAGCGATTTTATTATCTAACTGCTGATCGAGTGTAGCC |
| | AGATCTAGTAATCAATTACGGGGTCATTAGTTCATAGCCCATATATGGAGTTCCGCGTTACATAACT |
| | TACGGTAAATGGCCCGCCTGGCTGACCGCCCAACGACCCCCGCCCATTGACGTCAATAATGACGTAT |
| | GTTCCCATAGTAACGCCAATAGGGACTTTCCATTGACGTCAATGGGTGGAGTATTTACGGTAAACTG |
| | CCCACTTGGCAGTACATCAAGTGTATCATATGCCAAGTACGCCCCCTATTGACGTCAATGACGGTAA |
| | ATGGCCCGCCTGGCATTATGCCCAGTACATGACCTTATGGGACTTTCCTACTTGGCAGTACATCTAC |
| | GTATTAGTCATCGCTATTACCATGCTGATGCGGTTTTGGCAGTACATCAATGGGCGTGGATAGCGGT |
| | TTGACTCACGGGGATTTCCAAGTCTCCACCCCATTGACGTCAATGGGAGTTTGTTTTGGCACCAAAA |
| | TCAACGGGACTTTCCAAAATGTCGTAACAACTCCGCCCCATTGACGCAAATGGGCGGTAGGCGTGTA |
| | CGGTGGGAGGTCTATATAAGCAGAGCTGGTTTAGTGAACCGTCAGATCAGATCTTTGTCGATCCTAC |
| | CATCCACTCGACACACCCGCCAGCGGCCGCTAATACGACTCACTATAGGGAGAAGTACTGCCACCAT |
| | GGGCAAGAAGCAAAATCGCAAGACGGGGAATTCCAAGACACAATCCGCTAGCCCACCACCTAAAGAG |
| | CGTTCTAGCTCCCCTGCTACTGAGCAGTCCTGGATGGAAAACGACTTCGATGAACTCCGGGAAGAGG |
| | GATTTAGGCGATCCAACTATTCAGAACTCCGCGAAGATATCCAGACAAAGGGGAAGGAAGTCGAGAA |
| | TTTCGAGAAGAACCTCGAGGAGTGCATCACCCGTATCACAAACACTGAGAAATGTCTCAAAGAACTC |
| | ATGGAACTTAAGACAAAAGCCAGGGAGCTTCGAGAGGAGTGTCGGAGTCTGAGATCCAGGTGTGACC |
| | AGCTCGAGGAGCGCGTGAGCGCGATGGAAGACGAGATGAACGAGATGAAAAGAGAGGGCAAATTCAG |
| | GGAGAAGCGCATTAAGAGGAACGAACAGAGTCTGCAGGAGATTTGGGATTACGTCAAGAGGCCTAAC |
| | CTGCGGTTGATCGGCGTCCCCGAGAGCGACGTAGAAAACGGGACTAAACTGGAGAATACACTTCAAG |
| | ACATCATTCAAGAAAATTTTCCAAACCTGGCTCGGCAAGCTAATGTGCAAATCCAAGAGATCCAACG |
| | CACACCCCAGCGGTATAGCTCTCGGCGTGCCACCCCTAGGCATATTATCGTGCGCTTTACTAAGGTG |
| | GAGATGAAAGAGAAGATGCTGCGAGCCGCTCGGGAAAAGGGAAGGGTGACTTTGAAGGGCAAACCTA |
| | TTCGGCTGACGGTTGACCTTAGCGCCGAGACACTCCAGGCACGCCGGGAATGGGGCCCCATCTTTAA |
| | TATCCTGAAGGAGAAGAACTTCCAGCCACGAATCTCTTACCCTGCAAAGTTGAGTTTTATCTCCGAG |
| | GGTGAGATTAAGTATTTCATCGATAAACAGATGCTGCGAGACTTCGTGACAACTCGCCCAGCTCTCA |
| | AGGAACTGCTCAAAGAGGCTCTTAATATGGAGCGCAATAATAGATATCAACCCTTGCAGAACCACGC |
| | AAAGATGTGAGACAGCCGTCAGACCATCAAGACTAGGAAGAAACTGCATCAACTAATGAGCAAAATC |
| | ACCAGCTAACATCATAGTATACATGACCGGCTCTAACTCACATATCACCATCCTTACACTTAACATT |
| | AACGGCCTCAACTCAGCTATCAAGCGCCATCGGCTGGCCAGCTGGATCAAATCACAGGATCCAAGCG |
| | TTTGTTGCATCCAAGAGACCCACCTGACCTGTAGAGATACTCACCGCCTCAAGATCAAGGGATGGCG |
| | AAAGATTTATCAGGCGAACGGTAAGCAGAAGAAAGCCGGAGTCGCAATTCTGGTCTCAGACAAGACG |
| | GATTTCAAGCCCACCAAAATTAAGCGTGATAAGGAAGGTCACTATATTATGGTGAAAGGCAGCATAC |
| | AGCAGGAAGAACTTACCATATTGAACATCTACGCGCCAAACACCGGCGCACCTCGCTTTATCAAACA |
| | GGTCCTGTCCGATCTGCAGCGAGATCTGGATTCTCATACGTTGATTATGGGTGATTTCAATACACCA |
| | TTGAGCACCCTGGATCGCAGCACCAGGCAAAAGGTAAATAAAGACACGCAAGAGCTCAATAGCGCAC |
| | TGCATCAGGCAGATCTCATTGATATTTATCGCACTCTTCATCCTAAGAGTACCGAGTACACATTCTT |
| | CAGCGCCCCACATCATACATACTCAAAGATCGATCATATCGTCGGCTCAAAGGCTCTGCTGTCAAAG |
| | TGCAAGCGCACAGAGATAATTACAAATTACCTGTCAGATCATAGCGCGATCAAGCTCGAGCTGAGAA |
| | TCAAGAACCTGACCCAGAGCCGGAGTACCACTTGGAAGCTTAATAACCTGCTGCTCAACGATTATTG |
| | GGTCCACAATGAGATGAAGGCAGAGATTAAAATGTTCTTCGAAACAAATGAGAATAAGGATACTACC |
| | TATCAAAACCTTTGGGATGCCTTTAAGGCCGTCTGCAGAGGCAAGTTCATCGCCCTCAACGCCTATA |
| | AAAGAAAACAAGAGAGATCTAAGATCGATACTCTCACCTCTCAGCTGAAGGAGTTGGAGAAACAGGA |
| | ACAGACCCACTCCAAGGCGTCAAGACGGCAGGAGATCACAAAGATTCGCGCCGAGTTGAAAGAGATC |
| | GAAACCCAAAAGACTCTTCAGAAAATTAACGAGTCTCGTAGTTGGTTCTTCGAGCGGATTAATAAGA |
| | TAGACAGACCTCTGGCACGACTGATTAAGAAGAAGCGCGAAAAGAACCAGATTGATACCATCAAGAA |
| | CGACAAGGGCGACATCACTACTGACCCGACCGAGATCCAGACCACTATTCGGGAGTATTATAAGCAT |
| | TTGTATGCTAACAAGCTTGAGAACCTGGAAGAGATGGACACTTTTCTGGATACCTATACTCTGCCAC |
| | GGCTTAATCAAGAGGAAGTCGAGTCCCTCAACCGCCCAATTACAGGAAGCGAGATTGTGGCCATAAT |
| | TAACTCCCTGCCGACAAAGAAATCTCCTGGTCCGGACGGGTTTACAGCTGAGTTTTATCAACGGTAT |
| | ATGGAAGAGCTTGTACCGTTTCTGCTCAAGCTCTTTCAGTCTATAGAAAAGGAAGGCATCTTGCCCA |
| | ATTCCTTCTACGAAGCTTCTATAATACTTATTCCCAAACCAGGACGCGATACCACAAAGAAGGAAAA |
| | CTTCCGGCCCATTAGTCTCATGAATATCGACGCTAAAATATTGAACAAGATTCTCGCCAACAGAATC |
| | CAACAACATATTAAGAAATTGATACATCACGACCAGGTGGGGTTTATACCTGGCATGCAGGGCTGGT |
| | TTAACATCCGGAAGAGTATTAACGTCATTCAACACATTAATAGAGCTAAGGATAAGAATCATATGAT |
| | CATCTCTATAGACGCGGAAAAGGCATTCGATAAGATTCAGCAGCCATTTATGCTCAAGACTCTGAAC |
| | AAACTCGGCATCGACGGAACATATTTTAAGATTATTCGCGCAATTTACGATAAGCCGACTGCTAACA |
| | TTATCCTTAACGGCCAAAAGCTCGAGGCCTTTCCGCTCAAGACTGGAACCCGCCAAGGCTGTCCCCT |
| | CTCCCCGCTTTTGTTTAATATTGTACTCGAGGTGCTGGCTAGGGCTATTCGTCAAGAGAAAGAGATT |
| | AAAGGGATACAGCTCGGGAAGGAAGAGGTCAAGCTTTCCTTGTTCGCCGATGATATGATTGTGTACC |
| | TGGAGAATCCTATTGTGTCTGCTCAGAACCTTCTTAAACTTATTTCTAACTTTAGCAAGGTCAGCGG |
| | CTATAAGATTAACGTCCAGAAATCTCAGGCCTTTCTGTACACAAATAATCGACAGACCGAATCCCAG |
| | ATAATGGGTGAGCTTCCGTTTGTCATAGCCAGCAAAAGGATAAAGTATCTCGGAATCCAGCTGACAC |
| | GAGACGTTAAAGATTTGTTTAAGGAAAATTACAAGCCTCTCCTGAAAGAGATTAAGGAAGATACTAA |
| | TAAGTGGAAGAATATCCCCTGTTCATGGGTTGGCAGAATCAACATAGTGAAGATGGCAATACTTCCT |
| | AAAGTGATATATCGCTTTAACGCCATCCCAATTAAACTGCCTATGACCTTCTTTACGGAGCTCGAGA |
| | AAACAACCCTTAAATTTATATGGAATCAAAAGAGAGCAAGAATAGCGAAGTCCATCTTGAGCCAGAA |
| | GAATAAGGCCGGTGGGATTACTTTGCCTGATTTTAAGTTGTATTATAAAGCCACAGTAACTAAGACA |
| | GCCTGGTATTGGTATCAGAATAGAGACATCGACCAGTGGAATCGGACCGAACCATCAGAGATAATGC |
| | CCCACATCTATAATTACCTTATATTCGATAAGCCAGAAAAGAATAAACAGTGGGGCAAAGACAGCCT |
| | CTTCAACAAGTGGTGTTGGGAGAATTGGCTGGCCATATGCCGGAAACTCAAGCTCGACCCCTTTCTT |
| | ACACCCTACACTAAAATCAACAGTAGGTGGATCAAGGACTTGAATGTCAAGCCAAAGACTATAAAGA |
| | CACTGGAAGAGAATCTTGGGATCACAATACAAGATATAGGCGTCGGCAAAGATTTTATGTCAAAGAC |
| | GCCCAAGGCCATGGCCACTAAGGATAAGATTGATAAGTGGGACCTTATTAAGCTCAAAAGCTTCTGT |
| | ACTGCCAAGGAGACCACGATCAGAGTTAATAGGCAGCCCACTACATGGGAAAAGATTTTCGCCACTT |
| | ATTCATCAGATAAGGGGTTGATAAGCAGAATATATAACGAGCTGAAGCAGATCTACAAGAAGAAAAC |
| | GAATAATCCCATCAAGAAGTGGGCAAAAGATATGAACAGGCATTTTAGCAAAGAGGATATCTACGCC |
| | GCGAAGAAGCATATGAAGAAGTGTAGTTCAAGCTTGGCCATTCGTGAGATGCAGATTAAGACGACCA |
| | TGCGATACCACCTTACCCCAGTGAGGATGGCAATTATCAAGAAATCTGGCAATAATAGATGTTGGCG |
| | GGGCTGTGGCGAGATTGGCACCCTGCTCCATTGCTGGTGGGATTGCAAGCTGGTGCAGCCGCTTTGG |
| | AAATCAGTCTGGCGCTTTCTGAGGGACCTCGAGCTTGAGATTCCCTTCGATCCCGCAATTCCCTTGC |
| | TCGGAATCTATCCTAACGAATACAAGAGCTGTTGTTACAAGGATACGTGTACCCGGATGTTCATCGC |
| | GGCCTTGTTTACGATAGCTAAGACGTGGAATCAGCCTAAGTGCCCCACAATGATCGATTGGATCAAG |
| | AAAATGTGGCATATTTATACCATGGAGTATTACGCAGCAATTAAGAATGACGAATTTATTTCCTTCG |
| | TTGGGACCTGGATGAAGCTGGAGACTATTATTCTGAGCAAGCTGTCTCAGGAGCAAAAGACAAAGCA |
| | TAGAATCTTCTCTCTCATTGGTGGTAACGACTACAAAGACGATGACGACAAGggcggcggcagcaaa |
| | aggccggcggccacgaaaaaggccggccaggcaaaaaagaaaaagTAAAGCGCTTCTAGAAGTTGTC |
| | TCCTCCTGCACTGACTGACTGATACAATCGATTTCTGGATCCGCAGGCCTAATCAACCTCTGGATTA |
| | CAAAATTTGTGAAAGATTGACTGGTATTCTTAACTATGTTGCTCCTTTTACGCTATGTGGATACGCT |
| | GCTTTAATGCCTTTGTATCATGCTATTGCTTCCCGTATGGCTTTCATTTTCTCCTCCTTGTATAAAT |
| | CCTGGTTGCTGTCTCTTTATGAGGAGTTGTGGCCCGTTGTCAGGCAACGTGGCGTGGTGTGCACTGT |
| | GTTTGCTGACGCAACCCCCACTGGTTGGGGCATTGCCACCACCTGTCAGCTCCTTTCCGGGACTTTC |
| | GCTTTCCCCCTCCCTATTGCCACGGCGGAACTCATCGCCGCCTGCCTTGCCCGCTGCTGGACAGGGG |
| | CTCGGCTGTTGGGCACTGACAATTCCGTGGTGTTGTCGGGGAAGCTGACGTCCTTTCCATGGCTGCT |
| | CGCCTGTGTTGCCACCTGGATTCTGCGCGGGACGTCCTTCTGCTACGTCCCTTCGGCCCTCAATCCA |
| | GCGGACCTTCCTTCCCGCtgagagacacaaaaaattccaacacactattgcaatgaaaataaatttc |
| | ctttattagccagaagtcagatgctcaaggggcttcatgatgtccccataatttttggcagagggaa |
| | aaagatctcagtggtatttgtgagccagggcattggccttctgataggcagcctgcacctgaggagt |
| | gcggccgctttacttgtacagctcgtccatgccgagagtgatcccggcggcggtcacgaactccagc |
| | aggaccatgtgatcgcgcttctcgttggggtctttgctcagggcggactgggtgctcaggtagtggt |
| | tgtcgggcagcagcacggggccgtcgccgatgggggtgttctgctggtagtggtcggcgagctgcac |
| | gctgccgtcctcgatgttgtggcggatcttgaagttcaccttgatgccgttcttctgcttgtcggcc |
| | atgatatagacgttgtggctgttgtagttgtactccagcttgtgccccaggatgttgccgtcctcct |
| | tgaagtcgatgcccttcagctcgatgcggttcaccagggtgtcgccctcgaacttcacctcggcgcg |
| | ggtcttgtagttgccgtcgtccttgaagaagatggtgcgctcctggacgtagccttcgggcatggcg |
| | gacttgaagaagtcgtgctgcttcatgtggtcggggtagcggctgaagcactgcacgccgtaggtca |
| | gggtggtcacgagggtgggccagggcacgggcagcttgccggtggtgcagatgaacttcagggtcag |
| | cttgccgtaggtggcatcgccctcgccctcgccggacacgctgaacttgtggccgtttacgtcgccg |
| | tccagctcgaccaggatgggcaccaccccggtgaacagctcctcgcccttgctcaccatggtggcgg |
| | gatctgacggttcactaaaccagctctgcttatatagacctcccaccgtacacgcctaccgcccatt |
| | tgcgtcaatggggcggagttgttacgacattttggaaagtcccgttgattttggtgccaaaacaaac |
| | tcccattgacgtcaatggggtggagacttggaaatccccgtgagtcaaaccgctatccacgcccatt |
| | gatgtactgccaaaaccgcatcaccatggtaatagcgatgactaatacgtagatgtactgccaagta |
| | ggaaagtcccataaggtcatgtactgggcataatgccaggcgggccatttaccgtcattgacgtcaa |
| | tagggggcgtacttggcatatgatacacttgatgtactgccaagtgggcagtttaccgtaaatactc |
| | cacccattgacgtcaatggaaagtccctattggcgttactatgggaacatacgtcattattgacgtc |
| | aatgggcgggggtcgttgggcggtcagccaggcgggccatttaccgtaagttatgtaacgGGCCTGC |
| | TGCCGGCTCTGCGGCCTCTTCCGCGTCTTCGCCTTCGCCCTCAGACGAGTCGGATCTCCCTTTGGGC |
| | CGCCTCCCCGCCTGTCTAGCTTGACTGACTGAGATACAGCGTACCTTCAGCTCACAGACATGATAAG |
| | ATACATTGATGAGTTTGGACAAACCACAACTAGAATGCAGTGAAAAAAATGCTTTATTTGTGAAATT |
| | TGTGATGCTATTGCTTTATTTGTAACCATTATAAGCTGCAATAAACAAGTTAACAACAACAATTGCA |
| | TTCATTTTATGTTTCAGGTTCAGGGGGAGGTGTGGGAGGTTTTTTAAAGCAAGTAAAACCTCTACAA |
| | ATGTGGTATTGGCCCATCTCTATCGGTATCGTAGCATAACCCCTTGGGGCCTCTAAACGGGTCTTGA |
| | GGGGTTTTTTGTGCCCCTCGGGCCGGATTGCTATCTACCGGCATTGGCGCAGAAAAAAATGCCTGAT |
| | GCGACGCTGCGCGTCTTATACTCCCACATATGCCAGATTCAGCAACGGATACGGCTTCCCCAACTTG |
| | CCCACTTCCATACGTGTCCTCCTTACCAGAAATTTATCCTTAAGGTCGTCAGCTATCCTGCAGGCGA |
| | TCTCTCGATTTCGATCAAGACATTCCTTTAATGGTCTTTTCTGGACACCACTAGGGGTCAGAAGTAG |
| | TTCATCAAACTTTCTTCCCTCCCTAATCTCATTGGTTACCTTGGGCTATCGAAACTTAATTAAGCGA |
| | TCTGCATCTCAATTAGTCAGCAACCATAGTCCCGCCCCTAACTCCGCCCATCCCGCCCCTAACTCCG |
| | CCCAGTTCCGCCCATTCTCCGCCCCATCGCTGACTAATTTTTTTTATTTATGCAGAGGCCGAGGCCG |
| | CCTCGGCCTCTGAGCTATTCCAGAAGTAGTGAGGAGGCTTTTTTGGAGGCCTAGGCTTTTGCAAAGG |
| | AGGTAGCCAACATGATTGAACAAGATGGATTGCACGCAGGTTCTCCCGCCGCTTGGGTGGAGAGGCT |
| | ATTCGGCTATGACTGGGCACAACAGACAATCGGCTGCTCTGATGCCGCCGTGTTCCGGCTGTCAGCG |
| | CAGGGGCGCCCGGTTCTTTTTGTCAAGACCGACCTGTCCGGTGCCCTGAATGAACTCCAGGACGAGG |
| | CAGCGCGGCTATCGTGGCTGGCCACGACGGGCGTTCCTTGCGCAGCTGTGCTCGACGTTGTCACTGA |
| | AGCGGGAAGGGACTGGCTGCTATTGGGCGAAGTGCCGGGGCAGGATCTCCTGTCATCTCACCTTGCT |
| | CCTGCCGAGAAAGTATCCATCATGGCTGATGCAATGCGGCGGCTGCATACGCTTGATCCGGCTACCT |
| | GCCCATTCGACCACCAAGCGAAACATCGCATCGAGCGAGCACGTACTCGGATGGAAGCCGGTCTTGT |
| | CGATCAGGATGATCTGGACGAAGAGCATCAGGGGCTCGCGCCAGCCGAACTGTTCGCCAGGCTCAAG |
| | GCGCGGATGCCCGACGGCGAGGATCTCGTCGTGACCCACGGCGATGCCTGCTTGCCGAATATCATGG |
| | TGGAAAATGGCCGCTTTTCTGGATTCATCGACTGTGGCCGGCTGGGTGTGGCGGACCGCTATCAGGA |
| | CATAGCGTTGGCTACCCGTGATATTGCTGAAGAGCTTGGCGGCGAATGGGCTGACCGCTTCCTCGTG |
| | CTTTACGGTATCGCCGCTCCCGATTCGCAGCGCATCGCCTTCTATCGCCTTCTTGACGAGTTCTTCT |
| | AGTATGTAAGCCCTGTGCCTTCTAGTTGCCAGCCATCTGTTGTTTGCCCCTCCCCCGTGCCTTCCTT |
| | GACCCTGGAAGGTGCCACTCCCACTGTCCTTTCCTAATAAAATGAGGAAATTGCATCGCATTGTCTG |
| | AGTAGGTGTCATTCTATTCTGGGGGGTGGGGTGGGGCAGGACAGCAAGGGGGAGGATTGGGAAGACA |
| | ATAGCAGGCATGCTGGGGATGCGGTGGGCTCTATGGTTAATTAACCAGTCAAGTCAGCTACTTGGCG |
| | AGATCGACTTGTCTGGGTTTCGACTACGCTCAGAATTGCGTCAGTCAAGTTCGATCTGGTCCTTGCT |
| | ATTGCACCCGTTCTCCGATTACGAGTTTCATTTAAATCATGTGAGCAAAAGGCCAGCAAAAGGCCAG |
| | GAACCGTAAAAAGGCCGCGTTGCTGGCGTTTTTCCATAGGCTCCGCCCCCCTGACGAGCATCACAAA |
| | AATCGACGCTCAAGTCAGAGGTGGCGAAACCCGACAGGACTATAAAGATACCAGGCGTTTCCCCCTG |
| | GAAGCTCCCTCGTGCGCTCTCCTGTTCCGACCCTGCCGCTTACCGGATACCTGTCCGCCTTTCTCCC |
| | TTCGGGAAGCGTGGCGCTTTCTCATAGCTCACGCTGTAGGTATCTCAGTTCGGTGTAGGTCGTTCGC |
| | TCCAAGCTGGGCTGTGTGCACGAACCCCCCGTTCAGCCCGACCGCTGCGCCTTATCCGGTAACTATC |
| | GTCTTGAGTCCAACCCGGTAAGACACGACTTATCGCCACTGGCAGCAGCCACTGGTAACAGGATTAG |
| | CAGAGCGAGGTATGTAGGCGGTGCTACAGAGTTCTTGAAGTGGTGGCCTAACTACGGCTACACTAGA |
| | AGAACAGTATTTGGTATCTGCGCTCTGCTGAAGCCAGTTACCTTCGGAAAAAGAGTTGGTAGCTCTT |
| | GATCCGGCAAACAAACCACCGCTGGTAGCGGTGGTTTTTTTGTTTGCAAGCAGCAGATTACGCGCAG |
| | AAAAAAAGGATCTCAAGAAGATCCTTTGATCTTTTCTACGGGGTCTGACGCTCAGTGGAACGAAAAC |
| | TCACGTTAAGGGATTTTGGTCATGAGATTATCAAAAAGGATCTTCACCTAGATCCTTTTAAATTAAA |
| | AATGAAGTTTTAAATCAATCTAAAGTATATATGAGTAAACTTGGTCTGACAGTTACCAATGCTTAAT |
| | CAGTGAGGCACCTATCTCAGCGATCTGTCTATTTCGTTCATCCATAGTTGCATTTAAATTTCCGAAC |
| | TCTCCAAGGCCCTCGTCGGAAAATCTTCAAACCTTTCGTCCGATCCATCTTGCAGGCTACCTCTCGA |
| | ACGAACTATCGCAAGTCTCTTGGCCGGCCTTGCGCCTTGGCTATTGCTTGGCAGCGCCTATCGCCAG |
| | GTATTACTCCAATCCCGAATATCCGAGATCGGGATCACCCGAGAGAAGTTCAACCTACATCCTCAAT |
| | CCCGATCTATCCGAGATCCGAGGAATATCGAAATCGGGGCGCGCCTGGTGTA (SEQ ID NO: |
| | 107) |
| RET-018 | TGGCCGGCCTTGCGCCTTGGCTATTGCTTGGCAGCGCCTATCGCCAGGTATTACTCCAATCCCGAAT |
| LINE1- | ATCCGAGATCGGGATCACCCGAGAGAAGTTCAACCTACATCCTCAATCCCGATCTATCCGAGATCCG |
| GFP | AGGAATATCGAAATCGGGGCGCGCCTGGTGTACCGAGAACGATCCTCTCAGTGCGAGTCTCGACGAT |
| ORF2- | CCATATCGTTGCTTGGCAGTCAGCCAGTCGGAATCCAGCTTGGGACCCAGGAAGTCCAATCGTCAGA |
| N_SV40_ | TATTGTACTCAAGCCTGGTCACGGCAGCGTACCGATCTGTTTAAACCTAGATATTGATAGTCTGATC |
| NLS_ | GGTCAACGTATAATCGAGTCCTAGCTTTTGCAAACATCTATCAAGAGACAGGATCAGCAGGAGGCTT |
| Linker | TCGCATGAGTATTCAACATTTCCGTGTCGCCCTTATTCCCTTTTTTGCGGCATTTTGCCTTCCTGTT |
| ORF2- | TTTGCTCACCCAGAAACGCTGGTGAAAGTAAAAGATGCTGAAGATCAGTTGGGTGCGCGAGTGGGTT |
| C_Nucleo- | ACATCGAACTGGATCTCAACAGCGGTAAGATCCTTGAGAGTTTTCGCCCCGAAGAACGCTTTCCAAT |
| plasmin_ | GATGAGCACTTTTAAAGTTCTGCTATGTGGCGCGGTATTATCCCGTATTGACGCCGGGCAAGAGCAA |
| NLS | CTCGGTCGCCGCATACACTATTCTCAGAATGACTTGGTTGAGTATTCACCAGTCACAGAAAAGCATC |
| | TTACGGATGGCATGACAGTAAGAGAATTATGCAGTGCTGCCATAACCATGAGTGATAACACTGCGGC |
| | CAACTTACTTCTGACAACGATTGGAGGACCGAAGGAGCTAACCGCTTTTTTGCACAACATGGGGGAT |
| | CATGTAACTCGCCTTGATCGTTGGGAACCGGAGCTGAATGAAGCCATACCAAACGACGAGCGTGACA |
| | CCACGATGCCTGTAGCAATGGCAACAACCTTGCGTAAACTATTAACTGGCGAACTACTTACTCTAGC |
| | TTCCCGGCAACAGTTGATAGACTGGATGGAGGCGGATAAAGTTGCAGGACCACTTCTGCGCTCGGCC |
| | CTTCCGGCTGGCTGGTTTATTGCTGATAAATCTGGAGCCGGTGAGCGTGGGTCTCGCGGTATCATTG |
| | CAGCACTGGGGCCAGATGGTAAGCCCTCCCGTATCGTAGTTATCTACACGACGGGGAGTCAGGCAAC |
| | TATGGATGAACGAAATAGACAGATCGCTGAGATAGGTGCCTCACTGATTAAGCATTGGTAACCGATT |
| | CTAGGTGCATTGGCGCAGAAAAAAATGCCTGATGCGACGCTGCGCGTCTTATACTCCCACATATGCC |
| | AGATTCAGCAACGGATACGGCTTCCCCAACTTGCCCACTTCCATACGTGTCCTCCTTACCAGAAATT |
| | TATCCTTAAGATCGTTTAAACTCGACTCTGGCTCTATCGAATCTCCGTCGTTTCGAGCTTACGCGAA |
| | CAGCCGTGGCGCTCATTTGCTCGTCGGGCATCGAATCTCGTCAGCTATCGTCAGCTTACCTTTTTGG |
| | CAGCGATCGCGGCTCCCGACATCTTGGACCATTAGCTCCACAGGTATCTTCTTCCCTCTAGTGGTCA |
| | TAACAGCAGCTTCAGCTACCTCTCAATTCAAAAAACCCCTCAAGACCCGTTTAGAGGCCCCAAGGGG |
| | TTATGCTATCAATCGTTGCGTTACACACACAAAAAACCAACACACATCCATCTTCGATGGATAGCGA |
| | TTTTATTATCTAACTGCTGATCGAGTGTAGCCAGATCTAGTAATCAATTACGGGGTCATTAGTTCAT |
| | AGCCCATATATGGAGTTCCGCGTTACATAACTTACGGTAAATGGCCCGCCTGGCTGACCGCCCAACG |
| | ACCCCCGCCCATTGACGTCAATAATGACGTATGTTCCCATAGTAACGCCAATAGGGACTTTCCATTG |
| | ACGTCAATGGGTGGAGTATTTACGGTAAACTGCCCACTTGGCAGTACATCAAGTGTATCATATGCCA |
| | AGTACGCCCCCTATTGACGTCAATGACGGTAAATGGCCCGCCTGGCATTATGCCCAGTACATGACCT |
| | TATGGGACTTTCCTACTTGGCAGTACATCTACGTATTAGTCATCGCTATTACCATGCTGATGCGGTT |
| | TTGGCAGTACATCAATGGGCGTGGATAGCGGTTTGACTCACGGGGATTTCCAAGTCTCCACCCCATT |
| | GACGTCAATGGGAGTTTGTTTTGGCACCAAAATCAACGGGACTTTCCAAAATGTCGTAACAACTCCG |
| | CCCCATTGACGCAAATGGGCGGTAGGCGTGTACGGTGGGAGGTCTATATAAGCAGAGCTGGTTTAGT |
| | GAACCGTCAGATCAGATCTTTGTCGATCCTACCATCCACTCGACACACCCGCCAGCGGCCGCTAATA |
| | CGACTCACTATAGGGAGAAGTACTGCCACCATGGGCAAGAAGCAAAATCGCAAGACGGGGAATTCCA |
| | AGACACAATCCGCTAGCCCACCACCTAAAGAGCGTTCTAGCTCCCCTGCTACTGAGCAGTCCTGGAT |
| | GGAAAACGACTTCGATGAACTCCGGGAAGAGGGATTTAGGCGATCCAACTATTCAGAACTCCGCGAA |
| | GATATCCAGACAAAGGGGAAGGAAGTCGAGAATTTCGAGAAGAACCTCGAGGAGTGCATCACCCGTA |
| | TCACAAACACTGAGAAATGTCTCAAAGAACTCATGGAACTTAAGACAAAAGCCAGGGAGCTTCGAGA |
| | GGAGTGTCGGAGTCTGAGATCCAGGTGTGACCAGCTCGAGGAGCGCGTGAGCGCGATGGAAGACGAG |
| | ATGAACGAGATGAAAAGAGAGGGCAAATTCAGGGAGAAGCGCATTAAGAGGAACGAACAGAGTCTGC |
| | AGGAGATTTGGGATTACGTCAAGAGGCCTAACCTGCGGTTGATCGGCGTCCCCGAGAGCGACGTAGA |
| | AAACGGGACTAAACTGGAGAATACACTTCAAGACATCATTCAAGAAAATTTTCCAAACCTGGCTCGG |
| | CAAGCTAATGTGCAAATCCAAGAGATCCAACGCACACCCCAGCGGTATAGCTCTCGGCGTGCCACCC |
| | CTAGGCATATTATCGTGCGCTTTACTAAGGTGGAGATGAAAGAGAAGATGCTGCGAGCCGCTCGGGA |
| | AAAGGGAAGGGTGACTTTGAAGGGCAAACCTATTCGGCTGACGGTTGACCTTAGCGCCGAGACACTC |
| | CAGGCACGCCGGGAATGGGGCCCCATCTTTAATATCCTGAAGGAGAAGAACTTCCAGCCACGAATCT |
| | CTTACCCTGCAAAGTTGAGTTTTATCTCCGAGGGTGAGATTAAGTATTTCATCGATAAACAGATGCT |
| | GCGAGACTTCGTGACAACTCGCCCAGCTCTCAAGGAACTGCTCAAAGAGGCTCTTAATATGGAGCGC |
| | AATAATAGATATCAACCCTTGCAGAACCACGCAAAGATGTGAGACAGCCGTCAGACCATCAAGACTA |
| | GGAAGAAACTGCATCAACTAATGAGCAAAATCACCAGCTAACATCATAGTATACATGccaaagaaga |
| | agcggaaggtcggcggcggcagcACCGGCTCTAACTCACATATCACCATCCTTACACTTAACATTAA |
| | CGGCCTCAACTCAGCTATCAAGCGCCATCGGCTGGCCAGCTGGATCAAATCACAGGATCCAAGCGTT |
| | TGTTGCATCCAAGAGACCCACCTGACCTGTAGAGATACTCACCGCCTCAAGATCAAGGGATGGCGAA |
| | AGATTTATCAGGCGAACGGTAAGCAGAAGAAAGCCGGAGTCGCAATTCTGGTCTCAGACAAGACGGA |
| | TTTCAAGCCCACCAAAATTAAGCGTGATAAGGAAGGTCACTATATTATGGTGAAAGGCAGCATACAG |
| | CAGGAAGAACTTACCATATTGAACATCTACGCGCCAAACACCGGCGCACCTCGCTTTATCAAACAGG |
| | TCCTGTCCGATCTGCAGCGAGATCTGGATTCTCATACGTTGATTATGGGTGATTTCAATACACCATT |
| | GAGCACCCTGGATCGCAGCACCAGGCAAAAGGTAAATAAAGACACGCAAGAGCTCAATAGCGCACTG |
| | CATCAGGCAGATCTCATTGATATTTATCGCACTCTTCATCCTAAGAGTACCGAGTACACATTCTTCA |
| | GCGCCCCACATCATACATACTCAAAGATCGATCATATCGTCGGCTCAAAGGCTCTGCTGTCAAAGTG |
| | CAAGCGCACAGAGATAATTACAAATTACCTGTCAGATCATAGCGCGATCAAGCTCGAGCTGAGAATC |
| | AAGAACCTGACCCAGAGCCGGAGTACCACTTGGAAGCTTAATAACCTGCTGCTCAACGATTATTGGG |
| | TCCACAATGAGATGAAGGCAGAGATTAAAATGTTCTTCGAAACAAATGAGAATAAGGATACTACCTA |
| | TCAAAACCTTTGGGATGCCTTTAAGGCCGTCTGCAGAGGCAAGTTCATCGCCCTCAACGCCTATAAA |
| | AGAAAACAAGAGAGATCTAAGATCGATACTCTCACCTCTCAGCTGAAGGAGTTGGAGAAACAGGAAC |
| | AGACCCACTCCAAGGCGTCAAGACGGCAGGAGATCACAAAGATTCGCGCCGAGTTGAAAGAGATCGA |
| | AACCCAAAAGACTCTTCAGAAAATTAACGAGTCTCGTAGTTGGTTCTTCGAGCGGATTAATAAGATA |
| | GACAGACCTCTGGCACGACTGATTAAGAAGAAGCGCGAAAAGAACCAGATTGATACCATCAAGAACG |
| | ACAAGGGCGACATCACTACTGACCCGACCGAGATCCAGACCACTATTCGGGAGTATTATAAGCATTT |
| | GTATGCTAACAAGCTTGAGAACCTGGAAGAGATGGACACTTTTCTGGATACCTATACTCTGCCACGG |
| | CTTAATCAAGAGGAAGTCGAGTCCCTCAACCGCCCAATTACAGGAAGCGAGATTGTGGCCATAATTA |
| | ACTCCCTGCCGACAAAGAAATCTCCTGGTCCGGACGGGTTTACAGCTGAGTTTTATCAACGGTATAT |
| | GGAAGAGCTTGTACCGTTTCTGCTCAAGCTCTTTCAGTCTATAGAAAAGGAAGGCATCTTGCCCAAT |
| | TCCTTCTACGAAGCTTCTATAATACTTATTCCCAAACCAGGACGCGATACCACAAAGAAGGAAAACT |
| | TCCGGCCCATTAGTCTCATGAATATCGACGCTAAAATATTGAACAAGATTCTCGCCAACAGAATCCA |
| | ACAACATATTAAGAAATTGATACATCACGACCAGGTGGGGTTTATACCTGGCATGCAGGGCTGGTTT |
| | AACATCCGGAAGAGTATTAACGTCATTCAACACATTAATAGAGCTAAGGATAAGAATCATATGATCA |
| | TCTCTATAGACGCGGAAAAGGCATTCGATAAGATTCAGCAGCCATTTATGCTCAAGACTCTGAACAA |
| | ACTCGGCATCGACGGAACATATTTTAAGATTATTCGCGCAATTTACGATAAGCCGACTGCTAACATT |
| | ATCCTTAACGGCCAAAAGCTCGAGGCCTTTCCGCTCAAGACTGGAACCCGCCAAGGCTGTCCCCTCT |
| | CCCCGCTTTTGTTTAATATTGTACTCGAGGTGCTGGCTAGGGCTATTCGTCAAGAGAAAGAGATTAA |
| | AGGGATACAGCTCGGGAAGGAAGAGGTCAAGCTTTCCTTGTTCGCCGATGATATGATTGTGTACCTG |
| | GAGAATCCTATTGTGTCTGCTCAGAACCTTCTTAAACTTATTTCTAACTTTAGCAAGGTCAGCGGCT |
| | ATAAGATTAACGTCCAGAAATCTCAGGCCTTTCTGTACACAAATAATCGACAGACCGAATCCCAGAT |
| | AATGGGTGAGCTTCCGTTTGTCATAGCCAGCAAAAGGATAAAGTATCTCGGAATCCAGCTGACACGA |
| | GACGTTAAAGATTTGTTTAAGGAAAATTACAAGCCTCTCCTGAAAGAGATTAAGGAAGATACTAATA |
| | AGTGGAAGAATATCCCCTGTTCATGGGTTGGCAGAATCAACATAGTGAAGATGGCAATACTTCCTAA |
| | AGTGATATATCGCTTTAACGCCATCCCAATTAAACTGCCTATGACCTTCTTTACGGAGCTCGAGAAA |
| | ACAACCCTTAAATTTATATGGAATCAAAAGAGAGCAAGAATAGCGAAGTCCATCTTGAGCCAGAAGA |
| | ATAAGGCCGGTGGGATTACTTTGCCTGATTTTAAGTTGTATTATAAAGCCACAGTAACTAAGACAGC |
| | CTGGTATTGGTATCAGAATAGAGACATCGACCAGTGGAATCGGACCGAACCATCAGAGATAATGCCC |
| | CACATCTATAATTACCTTATATTCGATAAGCCAGAAAAGAATAAACAGTGGGGCAAAGACAGCCTCT |
| | TCAACAAGTGGTGTTGGGAGAATTGGCTGGCCATATGCCGGAAACTCAAGCTCGACCCCTTTCTTAC |
| | ACCCTACACTAAAATCAACAGTAGGTGGATCAAGGACTTGAATGTCAAGCCAAAGACTATAAAGACA |
| | CTGGAAGAGAATCTTGGGATCACAATACAAGATATAGGCGTCGGCAAAGATTTTATGTCAAAGACGC |
| | CCAAGGCCATGGCCACTAAGGATAAGATTGATAAGTGGGACCTTATTAAGCTCAAAAGCTTCTGTAC |
| | TGCCAAGGAGACCACGATCAGAGTTAATAGGCAGCCCACTACATGGGAAAAGATTTTCGCCACTTAT |
| | TCATCAGATAAGGGGTTGATAAGCAGAATATATAACGAGCTGAAGCAGATCTACAAGAAGAAAACGA |
| | ATAATCCCATCAAGAAGTGGGCAAAAGATATGAACAGGCATTTTAGCAAAGAGGATATCTACGCCGC |
| | GAAGAAGCATATGAAGAAGTGTAGTTCAAGCTTGGCCATTCGTGAGATGCAGATTAAGACGACCATG |
| | CGATACCACCTTACCCCAGTGAGGATGGCAATTATCAAGAAATCTGGCAATAATAGATGTTGGCGGG |
| | GCTGTGGCGAGATTGGCACCCTGCTCCATTGCTGGTGGGATTGCAAGCTGGTGCAGCCGCTTTGGAA |
| | ATCAGTCTGGCGCTTTCTGAGGGACCTCGAGCTTGAGATTCCCTTCGATCCCGCAATTCCCTTGCTC |
| | GGAATCTATCCTAACGAATACAAGAGCTGTTGTTACAAGGATACGTGTACCCGGATGTTCATCGCGG |
| | CCTTGTTTACGATAGCTAAGACGTGGAATCAGCCTAAGTGCCCCACAATGATCGATTGGATCAAGAA |
| | AATGTGGCATATTTATACCATGGAGTATTACGCAGCAATTAAGAATGACGAATTTATTTCCTTCGTT |
| | GGGACCTGGATGAAGCTGGAGACTATTATTCTGAGCAAGCTGTCTCAGGAGCAAAAGACAAAGCATA |
| | GAATCTTCTCTCTCATTGGTGGTAACGACTACAAAGACGATGACGACAAGaaaaggccggcggccac |
| | gaaaaaggccggccaggcaaaaaagaaaaagTAAAGCGCTTCTAGAAGTTGTCTCCTCCTGCACTGA |
| | CTGACTGATACAATCGATTTCTGGATCCGCAGGCCTAATCAACCTCTGGATTACAAAATTTGTGAAA |
| | GATTGACTGGTATTCTTAACTATGTTGCTCCTTTTACGCTATGTGGATACGCTGCTTTAATGCCTTT |
| | GTATCATGCTATTGCTTCCCGTATGGCTTTCATTTTCTCCTCCTTGTATAAATCCTGGTTGCTGTCT |
| | CTTTATGAGGAGTTGTGGCCCGTTGTCAGGCAACGTGGCGTGGTGTGCACTGTGTTTGCTGACGCAA |
| | CCCCCACTGGTTGGGGCATTGCCACCACCTGTCAGCTCCTTTCCGGGACTTTCGCTTTCCCCCTCCC |
| | TATTGCCACGGCGGAACTCATCGCCGCCTGCCTTGCCCGCTGCTGGACAGGGGCTCGGCTGTTGGGC |
| | ACTGACAATTCCGTGGTGTTGTCGGGGAAGCTGACGTCCTTTCCATGGCTGCTCGCCTGTGTTGCCA |
| | CCTGGATTCTGCGCGGGACGTCCTTCTGCTACGTCCCTTCGGCCCTCAATCCAGCGGACCTTCCTTC |
| | CCGCtgagagacacaaaaaattccaacacactattgcaatgaaaataaatttcctttattagccaga |
| | agtcagatgctcaaggggcttcatgatgtccccataatttttggcagagggaaaaagatctcagtgg |
| | tatttgtgagccagggcattggccttctgataggcagcctgcacctgaggagtgcggccgctttact |
| | tgtacagctcgtccatgccgagagtgatcccggcggcggtcacgaactccagcaggaccatgtgatc |
| | gcgcttctcgttggggtctttgctcagggcggactgggtgctcaggtagtggttgtcgggcagcagc |
| | acggggccgtcgccgatgggggtgttctgctggtagtggtcggcgagctgcacgctgccgtcctcga |
| | tgttgtggcggatcttgaagttcaccttgatgccgttcttctgcttgtcggccatgatatagacgtt |
| | gtggctgttgtagttgtactccagcttgtgccccaggatgttgccgtcctccttgaagtcgatgccc |
| | ttcagctcgatgcggttcaccagggtgtcgccctcgaacttcacctcggcgcgggtcttgtagttgc |
| | cgtcgtccttgaagaagatggtgcgctcctggacgtagccttcgggcatggcggacttgaagaagtc |
| | gtgctgcttcatgtggtcggggtagcggctgaagcactgcacgccgtaggtcagggtggtcacgagg |
| | gtgggccagggcacgggcagcttgccggtggtgcagatgaacttcagggtcagcttgccgtaggtgg |
| | catcgccctcgccctcgccggacacgctgaacttgtggccgtttacgtcgccgtccagctcgaccag |
| | gatgggcaccaccccggtgaacagctcctcgcccttgctcaccatggtggcgggatctgacggttca |
| | ctaaaccagctctgcttatatagacctcccaccgtacacgcctaccgcccatttgcgtcaatggggc |
| | ggagttgttacgacattttggaaagtcccgttgattttggtgccaaaacaaactcccattgacgtca |
| | atggggtggagacttggaaatccccgtgagtcaaaccgctatccacgcccattgatgtactgccaaa |
| | accgcatcaccatggtaatagcgatgactaatacgtagatgtactgccaagtaggaaagtcccataa |
| | ggtcatgtactgggcataatgccaggcgggccatttaccgtcattgacgtcaatagggggcgtactt |
| | ggcatatgatacacttgatgtactgccaagtgggcagtttaccgtaaatactccacccattgacgtc |
| | aatggaaagtccctattggcgttactatgggaacatacgtcattattgacgtcaatgggcgggggtc |
| | gttgggcggtcagccaggcgggccatttaccgtaagttatgtaacgGGCCTGCTGCCGGCTCTGCGG |
| | CCTCTTCCGCGTCTTCGCCTTCGCCCTCAGACGAGTCGGATCTCCCTTTGGGCCGCCTCCCCGCCTG |
| | TCTAGCTTGACTGACTGAGATACAGCGTACCTTCAGCTCACAGACATGATAAGATACATTGATGAGT |
| | TTGGACAAACCACAACTAGAATGCAGTGAAAAAAATGCTTTATTTGTGAAATTTGTGATGCTATTGC |
| | TTTATTTGTAACCATTATAAGCTGCAATAAACAAGTTAACAACAACAATTGCATTCATTTTATGTTT |
| | CAGGTTCAGGGGGAGGTGTGGGAGGTTTTTTAAAGCAAGTAAAACCTCTACAAATGTGGTATTGGCC |
| | CATCTCTATCGGTATCGTAGCATAACCCCTTGGGGCCTCTAAACGGGTCTTGAGGGGTTTTTTGTGC |
| | CCCTCGGGCCGGATTGCTATCTACCGGCATTGGCGCAGAAAAAAATGCCTGATGCGACGCTGCGCGT |
| | CTTATACTCCCACATATGCCAGATTCAGCAACGGATACGGCTTCCCCAACTTGCCCACTTCCATACG |
| | TGTCCTCCTTACCAGAAATTTATCCTTAAGGTCGTCAGCTATCCTGCAGGCGATCTCTCGATTTCGA |
| | TCAAGACATTCCTTTAATGGTCTTTTCTGGACACCACTAGGGGTCAGAAGTAGTTCATCAAACTTTC |
| | TTCCCTCCCTAATCTCATTGGTTACCTTGGGCTATCGAAACTTAATTAAGCGATCTGCATCTCAATT |
| | AGTCAGCAACCATAGTCCCGCCCCTAACTCCGCCCATCCCGCCCCTAACTCCGCCCAGTTCCGCCCA |
| | TTCTCCGCCCCATCGCTGACTAATTTTTTTTATTTATGCAGAGGCCGAGGCCGCCTCGGCCTCTGAG |
| | CTATTCCAGAAGTAGTGAGGAGGCTTTTTTGGAGGCCTAGGCTTTTGCAAAGGAGGTAGCCAACATG |
| | ATTGAACAAGATGGATTGCACGCAGGTTCTCCCGCCGCTTGGGTGGAGAGGCTATTCGGCTATGACT |
| | GGGCACAACAGACAATCGGCTGCTCTGATGCCGCCGTGTTCCGGCTGTCAGCGCAGGGGCGCCCGGT |
| | TCTTTTTGTCAAGACCGACCTGTCCGGTGCCCTGAATGAACTCCAGGACGAGGCAGCGCGGCTATCG |
| | TGGCTGGCCACGACGGGCGTTCCTTGCGCAGCTGTGCTCGACGTTGTCACTGAAGCGGGAAGGGACT |
| | GGCTGCTATTGGGCGAAGTGCCGGGGCAGGATCTCCTGTCATCTCACCTTGCTCCTGCCGAGAAAGT |
| | ATCCATCATGGCTGATGCAATGCGGCGGCTGCATACGCTTGATCCGGCTACCTGCCCATTCGACCAC |
| | CAAGCGAAACATCGCATCGAGCGAGCACGTACTCGGATGGAAGCCGGTCTTGTCGATCAGGATGATC |
| | TGGACGAAGAGCATCAGGGGCTCGCGCCAGCCGAACTGTTCGCCAGGCTCAAGGCGCGGATGCCCGA |
| | CGGCGAGGATCTCGTCGTGACCCACGGCGATGCCTGCTTGCCGAATATCATGGTGGAAAATGGCCGC |
| | TTTTCTGGATTCATCGACTGTGGCCGGCTGGGTGTGGCGGACCGCTATCAGGACATAGCGTTGGCTA |
| | CCCGTGATATTGCTGAAGAGCTTGGCGGCGAATGGGCTGACCGCTTCCTCGTGCTTTACGGTATCGC |
| | CGCTCCCGATTCGCAGCGCATCGCCTTCTATCGCCTTCTTGACGAGTTCTTCTAGTATGTAAGCCCT |
| | GTGCCTTCTAGTTGCCAGCCATCTGTTGTTTGCCCCTCCCCCGTGCCTTCCTTGACCCTGGAAGGTG |
| | CCACTCCCACTGTCCTTTCCTAATAAAATGAGGAAATTGCATCGCATTGTCTGAGTAGGTGTCATTC |
| | TATTCTGGGGGGTGGGGTGGGGCAGGACAGCAAGGGGGAGGATTGGGAAGACAATAGCAGGCATGCT |
| | GGGGATGCGGTGGGCTCTATGGTTAATTAACCAGTCAAGTCAGCTACTTGGCGAGATCGACTTGTCT |
| | GGGTTTCGACTACGCTCAGAATTGCGTCAGTCAAGTTCGATCTGGTCCTTGCTATTGCACCCGTTCT |
| | CCGATTAGGAGTTTCATTTAAATCATGTGAGCAAAAGGCCAGCAAAAGGCCAGGAACCGTAAAAAGG |
| | CCGCGTTGCTGGCGTTTTTCCATAGGCTCCGCCCCCCTGACGAGCATCACAAAAATCGACGCTCAAG |
| | TCAGAGGTGGCGAAACCCGACAGGACTATAAAGATACCAGGCGTTTCCCCCTGGAAGCTCCCTCGTG |
| | CGCTCTCCTGTTCCGACCCTGCCGCTTACCGGATACCTGTCCGCCTTTCTCCCTTCGGGAAGCGTGG |
| | CGCTTTCTCATAGCTCACGCTGTAGGTATCTCAGTTCGGTGTAGGTCGTTCGCTCCAAGCTGGGCTG |
| | TGTGCACGAACCCCCCGTTCAGCCCGACCGCTGCGCCTTATCCGGTAACTATCGTCTTGAGTCCAAC |
| | CCGGTAAGACACGACTTATCGCCACTGGCAGCAGCCACTGGTAACAGGATTAGCAGAGCGAGGTATG |
| | TAGGCGGTGCTACAGAGTTCTTGAAGTGGTGGCCTAACTACGGCTACACTAGAAGAACAGTATTTGG |
| | TATCTGCGCTCTGCTGAAGCCAGTTACCTTCGGAAAAAGAGTTGGTAGCTCTTGATCCGGCAAACAA |
| | ACCACCGCTGGTAGCGGTGGTTTTTTTGTTTGCAAGCAGCAGATTACGCGCAGAAAAAAAGGATCTC |
| | AAGAAGATCCTTTGATCTTTTCTACGGGGTCTGACGCTCAGTGGAACGAAAACTCACGTTAAGGGAT |
| | TTTGGTCATGAGATTATCAAAAAGGATCTTCACCTAGATCCTTTTAAATTAAAAATGAAGTTTTAAA |
| | TCAATCTAAAGTATATATGAGTAAACTTGGTCTGACAGTTACCAATGCTTAATCAGTGAGGCACCTA |
| | TCTCAGCGATCTGTCTATTTCGTTCATCCATAGTTGCATTTAAATTTCCGAACTCTCCAAGGCCCTC |
| | GTCGGAAAATCTTCAAACCTTTCGTCCGATCCATCTTGCAGGCTACCTCTCGAACGAACTATCGCAA |
| | GTCTCT (SEQ ID NO: 108) |
| ret-028- | TGGCCGGCCTTGCGCCTTGGCTATTGCTTGGCAGCGCCTATCGCCAGGTATTACTCCAATCCCGAAT |
| linel- | ATCCGAGATCGGGATCACCCGAGAGAAGTTCAACCTACATCCTCAATCCCGATCTATCCGAGATCCG |
| orf1-t2a- | AGGAATATCGAAATCGGGGCGCGCCTGGTGTACCGAGAACGATCCTCTCAGTGCGAGTCTCGACGAT |
| orf2- | CCATATCGTTGCTTGGCAGTCAGCCAGTCGGAATCCAGCTTGGGACCCAGGAAGTCCAATCGTCAGA |
| n_sv40_ | TATTGTACTCAAGCCTGGTCACGGCAGCGTACCGATCTGTTTAAACCTAGATATTGATAGTCTGATC |
| nls_linker- | GGTCAACGTATAATCGAGTCCTAGCTTTTGCAAACATCTATCAAGAGACAGGATCAGCAGGAGGCTT |
| gfp | TCGCATGAGTATTCAACATTTCCGTGTCGCCCTTATTCCCTTTTTTGCGGCATTTTGCCTTCCTGTT |
| | TTTGCTCACCCAGAAACGCTGGTGAAAGTAAAAGATGCTGAAGATCAGTTGGGTGCGCGAGTGGGTT |
| | ACATCGAACTGGATCTCAACAGCGGTAAGATCCTTGAGAGTTTTCGCCCCGAAGAACGCTTTCCAAT |
| | GATGAGCACTTTTAAAGTTCTGCTATGTGGCGCGGTATTATCCCGTATTGACGCCGGGCAAGAGCAA |
| | CTCGGTCGCCGCATACACTATTCTCAGAATGACTTGGTTGAGTATTCACCAGTCACAGAAAAGCATC |
| | TTACGGATGGCATGACAGTAAGAGAATTATGCAGTGCTGCCATAACCATGAGTGATAACACTGCGGC |
| | CAACTTACTTCTGACAACGATTGGAGGACCGAAGGAGCTAACCGCTTTTTTGCACAACATGGGGGAT |
| | CATGTAACTCGCCTTGATCGTTGGGAACCGGAGCTGAATGAAGCCATACCAAACGACGAGCGTGACA |
| | CCACGATGCCTGTAGCAATGGCAACAACCTTGCGTAAACTATTAACTGGCGAACTACTTACTCTAGC |
| | TTCCCGGCAACAGTTGATAGACTGGATGGAGGCGGATAAAGTTGCAGGACCACTTCTGCGCTCGGCC |
| | CTTCCGGCTGGCTGGTTTATTGCTGATAAATCTGGAGCCGGTGAGCGTGGGTCTCGCGGTATCATTG |
| | CAGCACTGGGGCCAGATGGTAAGCCCTCCCGTATCGTAGTTATCTACACGACGGGGAGTCAGGCAAC |
| | TATGGATGAACGAAATAGACAGATCGCTGAGATAGGTGCCTCACTGATTAAGCATTGGTAACCGATT |
| | CTAGGTGCATTGGCGCAGAAAAAAATGCCTGATGCGACGCTGCGCGTCTTATACTCCCACATATGCC |
| | AGATTCAGCAACGGATACGGCTTCCCCAACTTGCCCACTTCCATACGTGTCCTCCTTACCAGAAATT |
| | TATCCTTAAGATCGTTTAAACTCGACTCTGGCTCTATCGAATCTCCGTCGTTTCGAGCTTACGCGAA |
| | CAGCCGTGGCGCTCATTTGCTCGTCGGGCATCGAATCTCGTCAGCTATCGTCAGCTTACCTTTTTGG |
| | CAGCGATCGCGGCTCCCGACATCTTGGACCATTAGCTCCACAGGTATCTTCTTCCCTCTAGTGGTCA |
| | TAACAGCAGCTTCAGCTACCTCTCAATTCAAAAAACCCCTCAAGACCCGTTTAGAGGCCCCAAGGGG |
| | TTATGCTATCAATCGTTGCGTTACACACACAAAAAACCAACACACATCCATCTTCGATGGATAGCGA |
| | TTTTATTATCTAACTGCTGATCGAGTGTAGCCAGATCTAGTAATCAATTACGGGGTCATTAGTTCAT |
| | AGCCCATATATGGAGTTCCGCGTTACATAACTTACGGTAAATGGCCCGCCTGGCTGACCGCCCAACG |
| | ACCCCCGCCCATTGACGTCAATAATGACGTATGTTCCCATAGTAACGCCAATAGGGACTTTCCATTG |
| | ACGTCAATGGGTGGAGTATTTACGGTAAACTGCCCACTTGGCAGTACATCAAGTGTATCATATGCCA |
| | AGTACGCCCCCTATTGACGTCAATGACGGTAAATGGCCCGCCTGGCATTATGCCCAGTACATGACCT |
| | TATGGGACTTTCCTACTTGGCAGTACATCTACGTATTAGTCATCGCTATTACCATGCTGATGCGGTT |
| | TTGGCAGTACATCAATGGGCGTGGATAGCGGTTTGACTCACGGGGATTTCCAAGTCTCCACCCCATT |
| | GACGTCAATGGGAGTTTGTTTTGGCACCAAAATCAACGGGACTTTCCAAAATGTCGTAACAACTCCG |
| | CCCCATTGACGCAAATGGGCGGTAGGCGTGTACGGTGGGAGGTCTATATAAGCAGAGCTGGTTTAGT |
| | GAACCGTCAGATCAGATCTTTGTCGATCCTACCATCCACTCGACACACCCGCCAGCGGCCGCTAATA |
| | CGACTCACTATAGGGAGAAGTACTGCCACCATGGGCAAGAAGCAAAATCGCAAGACGGGGAATTCCA |
| | AGACACAATCCGCTAGCCCACCACCTAAAGAGCGTTCTAGCTCCCCTGCTACTGAGCAGTCCTGGAT |
| | GGAAAACGACTTCGATGAACTCCGGGAAGAGGGATTTAGGCGATCCAACTATTCAGAACTCCGCGAA |
| | GATATCCAGACAAAGGGGAAGGAAGTCGAGAATTTCGAGAAGAACCTCGAGGAGTGCATCACCCGTA |
| | TCACAAACACTGAGAAATGTCTCAAAGAACTCATGGAACTTAAGACAAAAGCCAGGGAGCTTCGAGA |
| | GGAGTGTCGGAGTCTGAGATCCAGGTGTGACCAGCTCGAGGAGCGCGTGAGCGCGATGGAAGACGAG |
| | ATGAACGAGATGAAAAGAGAGGGCAAATTCAGGGAGAAGCGCATTAAGAGGAACGAACAGAGTCTGC |
| | AGGAGATTTGGGATTACGTCAAGAGGCCTAACCTGCGGTTGATCGGCGTCCCCGAGAGCGACGTAGA |
| | AAACGGGACTAAACTGGAGAATACACTTCAAGACATCATTCAAGAAAATTTTCCAAACCTGGCTCGG |
| | CAAGCTAATGTGCAAATCCAAGAGATCCAACGCACACCCCAGCGGTATAGCTCTCGGCGTGCCACCC |
| | CTAGGCATATTATCGTGCGCTTTACTAAGGTGGAGATGAAAGAGAAGATGCTGCGAGCCGCTCGGGA |
| | AAAGGGAAGGGTGACTTTGAAGGGCAAACCTATTCGGCTGACGGTTGACCTTAGCGCCGAGACACTC |
| | CAGGCACGCCGGGAATGGGGCCCCATCTTTAATATCCTGAAGGAGAAGAACTTCCAGCCACGAATCT |
| | CTTACCCTGCAAAGTTGAGTTTTATCTCCGAGGGTGAGATTAAGTATTTCATCGATAAACAGATGCT |
| | GCGAGACTTCGTGACAACTCGCCCAGCTCTCAAGGAACTGCTCAAAGAGGCTCTTAATATGGAGCGC |
| | AATAATAGATATCAACCCTTGCAGAACCACGCAAAGATGggctccggcgagggcaggggaagCcttc |
| | taacatgcggggacgtggaggaaaatcccggcccaGGTAGCGGCccaaagaagaagcggaaggtcgg |
| | cggcggcagcACCGGCTCTAACTCACATATCACCATCCTTACACTTAACATTAACGGCCTCAACTCA |
| | GCTATCAAGCGCCATCGGCTGGCCAGCTGGATCAAATCACAGGATCCAAGCGTTTGTTGCATCCAAG |
| | AGACCCACCTGACCTGTAGAGATACTCACCGCCTCAAGATCAAGGGATGGCGAAAGATTTATCAGGC |
| | GAACGGTAAGCAGAAGAAAGCCGGAGTCGCAATTCTGGTCTCAGACAAGACGGATTTCAAGCCCACC |
| | AAAATTAAGCGTGATAAGGAAGGTCACTATATTATGGTGAAAGGCAGCATACAGCAGGAAGAACTTA |
| | CCATATTGAACATCTACGCGCCAAACACCGGCGCACCTCGCTTTATCAAACAGGTCCTGTCCGATCT |
| | GCAGCGAGATCTGGATTCTCATACGTTGATTATGGGTGATTTCAATACACCATTGAGCACCCTGGAT |
| | CGCAGCACCAGGCAAAAGGTAAATAAAGACACGCAAGAGCTCAATAGCGCACTGCATCAGGCAGATC |
| | TCATTGATATTTATCGCACTCTTCATCCTAAGAGTACCGAGTACACATTCTTCAGCGCCCCACATCA |
| | TACATACTCAAAGATCGATCATATCGTCGGCTCAAAGGCTCTGCTGTCAAAGTGCAAGCGCACAGAG |
| | ATAATTACAAATTAGCTGTCAGATCATAGCGCGATCAAGCTCGAGCTGAGAATCAAGAACCTGACCC |
| | AGAGCCGGAGTACCACTTGGAAGCTTAATAACCTGCTGCTCAACGATTATTGGGTCCACAATGAGAT |
| | GAAGGCAGAGATTAAAATGTTCTTCGAAACAAATGAGAATAAGGATACTACCTATCAAAACCTTTGG |
| | GATGCCTTTAAGGCCGTCTGCAGAGGCAAGTTCATCGCCCTCAACGCCTATAAAAGAAAACAAGAGA |
| | GATCTAAGATCGATACTCTCACCTCTCAGCTGAAGGAGTTGGAGAAACAGGAACAGACCCACTCCAA |
| | GGCGTCAAGACGGCAGGAGATCACAAAGATTCGCGCCGAGTTGAAAGAGATCGAAACCCAAAAGACT |
| | CTTCAGAAAATTAACGAGTCTCGTAGTTGGTTCTTCGAGCGGATTAATAAGATAGACAGACCTCTGG |
| | CACGACTGATTAAGAAGAAGCGCGAAAAGAACCAGATTGATACCATCAAGAACGACAAGGGCGACAT |
| | CACTACTGACCCGACCGAGATCCAGACCACTATTCGGGAGTATTATAAGCATTTGTATGCTAACAAG |
| | CTTGAGAACCTGGAAGAGATGGACACTTTTCTGGATACCTATACTCTGCCACGGCTTAATCAAGAGG |
| | AAGTCGAGTCCCTCAACCGCCCAATTACAGGAAGCGAGATTGTGGCCATAATTAACTCCCTGCCGAC |
| | AAAGAAATCTCCTGGTCCGGACGGGTTTACAGCTGAGTTTTATCAACGGTATATGGAAGAGCTTGTA |
| | CCGTTTCTGCTCAAGCTCTTTCAGTCTATAGAAAAGGAAGGCATCTTGCCCAATTCCTTCTACGAAG |
| | CTTCTATAATACTTATTCCCAAACCAGGACGCGATACCACAAAGAAGGAAAACTTCCGGCCCATTAG |
| | TCTCATGAATATCGACGCTAAAATATTGAACAAGATTCTCGCCAACAGAATCCAACAACATATTAAG |
| | AAATTGATACATCACGACCAGGTGGGGTTTATACCTGGCATGCAGGGCTGGTTTAACATCCGGAAGA |
| | GTATTAACGTCATTCAACACATTAATAGAGCTAAGGATAAGAATCATATGATCATCTCTATAGACGC |
| | GGAAAAGGCATTCGATAAGATTCAGCAGCCATTTATGCTCAAGACTCTGAACAAACTCGGCATCGAC |
| | GGAACATATTTTAAGATTATTCGCGCAATTTACGATAAGCCGACTGCTAACATTATCCTTAACGGCC |
| | AAAAGCTCGAGGCCTTTCCGCTCAAGACTGGAACCCGCCAAGGCTGTCCCCTCTCCCCGCTTTTGTT |
| | TAATATTGTACTCGAGGTGCTGGCTAGGGCTATTCGTCAAGAGAAAGAGATTAAAGGGATACAGCTC |
| | GGGAAGGAAGAGGTCAAGCTTTCCTTGTTCGCCGATGATATGATTGTGTACCTGGAGAATCCTATTG |
| | TGTCTGCTCAGAACCTTCTTAAACTTATTTCTAACTTTAGCAAGGTCAGCGGCTATAAGATTAACGT |
| | CCAGAAATCTCAGGCCTTTCTGTACACAAATAATCGACAGACCGAATCCCAGATAATGGGTGAGCTT |
| | CCGTTTGTCATAGCCAGCAAAAGGATAAAGTATCTCGGAATCCAGCTGACACGAGACGTTAAAGATT |
| | TGTTTAAGGAAAATTACAAGCCTCTCCTGAAAGAGATTAAGGAAGATACTAATAAGTGGAAGAATAT |
| | CCCCTGTTCATGGGTTGGCAGAATCAACATAGTGAAGATGGCAATACTTCCTAAAGTGATATATCGC |
| | TTTAACGCCATCCCAATTAAACTGCCTATGACCTTCTTTACGGAGCTCGAGAAAACAACCCTTAAAT |
| | TTATATGGAATCAAAAGAGAGCAAGAATAGCGAAGTCCATCTTGAGCCAGAAGAATAAGGCCGGTGG |
| | GATTACTTTGCCTGATTTTAAGTTGTATTATAAAGCCACAGTAACTAAGACAGCCTGGTATTGGTAT |
| | CAGAATAGAGACATCGACCAGTGGAATCGGACCGAACCATCAGAGATAATGCCCCACATCTATAATT |
| | ACCTTATATTCGATAAGCCAGAAAAGAATAAACAGTGGGGCAAAGACAGCCTCTTCAACAAGTGGTG |
| | TTGGGAGAATTGGCTGGCCATATGCCGGAAACTCAAGCTCGACCCCTTTCTTACACCCTACACTAAA |
| | ATCAACAGTAGGTGGATCAAGGACTTGAATGTCAAGCCAAAGACTATAAAGACACTGGAAGAGAATC |
| | TTGGGATCACAATACAAGATATAGGCGTCGGCAAAGATTTTATGTCAAAGACGCCCAAGGCCATGGC |
| | CACTAAGGATAAGATTGATAAGTGGGACCTTATTAAGCTCAAAAGCTTCTGTACTGCCAAGGAGACC |
| | ACGATCAGAGTTAATAGGCAGCCCACTACATGGGAAAAGATTTTCGCCACTTATTCATCAGATAAGG |
| | GGTTGATAAGCAGAATATATAACGAGCTGAAGCAGATCTACAAGAAGAAAACGAATAATCCCATCAA |
| | GAAGTGGGCAAAAGATATGAACAGGCATTTTAGCAAAGAGGATATCTACGCCGCGAAGAAGCATATG |
| | AAGAAGTGTAGTTCAAGCTTGGCCATTCGTGAGATGCAGATTAAGACGACCATGCGATACCACCTTA |
| | CCCCAGTGAGGATGGCAATTATCAAGAAATCTGGCAATAATAGATGTTGGCGGGGCTGTGGCGAGAT |
| | TGGCACCCTGCTCCATTGCTGGTGGGATTGCAAGCTGGTGCAGCCGCTTTGGAAATCAGTCTGGCGC |
| | TTTCTGAGGGACCTCGAGCTTGAGATTCCCTTCGATCCCGCAATTCCCTTGCTCGGAATCTATCCTA |
| | ACGAATACAAGAGCTGTTGTTACAAGGATACGTGTACCCGGATGTTCATCGCGGCCTTGTTTACGAT |
| | AGCTAAGACGTGGAATCAGCCTAAGTGCCCCACAATGATCGATTGGATCAAGAAAATGTGGCATATT |
| | TATACCATGGAGTATTACGCAGCAATTAAGAATGACGAATTTATTTCCTTCGTTGGGACCTGGATGA |
| | AGCTGGAGACTATTATTCTGAGCAAGCTGTCTCAGGAGCAAAAGACAAAGCATAGAATCTTCTCTCT |
| | CATTGGTGGTAACGACTACAAAGACGATGACGACAAGTAAAGCGCTTCTAGAAGTTGTCTCCTCCTG |
| | CACTGACTGACTGATACAATCGATTTCTGGATCCGCAGGCCTAATCAACCTCTGGATTACAAAATTT |
| | GTGAAAGATTGACTGGTATTCTTAACTATGTTGCTCCTTTTACGCTATGTGGATACGCTGCTTTAAT |
| | GCCTTTGTATCATGCTATTGCTTCCCGTATGGCTTTCATTTTCTCCTCCTTGTATAAATCCTGGTTG |
| | CTGTCTCTTTATGAGGAGTTGTGGCCCGTTGTCAGGCAACGTGGCGTGGTGTGCACTGTGTTTGCTG |
| | ACGCAACCCCCACTGGTTGGGGCATTGCCACCACCTGTCAGCTCCTTTCCGGGACTTTCGCTTTCCC |
| | CCTCCCTATTGCCACGGCGGAACTCATCGCCGCCTGCCTTGCCCGCTGCTGGACAGGGGCTCGGCTG |
| | TTGGGCACTGACAATTCCGTGGTGTTGTCGGGGAAGCTGACGTCCTTTCCATGGCTGCTCGCCTGTG |
| | TTGCCACCTGGATTCTGCGCGGGACGTCCTTCTGCTACGTCCCTTCGGCCCTCAATCCAGCGGACCT |
| | TCCTTCCCGCtgagagacacaaaaaattccaacacactattgcaatgaaaataaatttcctttatta |
| | gccagaagtcagatgctcaaggggcttcatgatgtccccataatttttggcagagggaaaaagatct |
| | cagtggtatttgtgagccagggcattggccttctgataggcagcctgcacctgaggagtgcggccgc |
| | tttacttgtacagctcgtccatgccgagagtgatcccggcggcggtcacgaactccagcaggaccat |
| | gtgatcgcgcttctcgttggggtctttgctcagggcggactgggtgctcaggtagtggttgtcgggc |
| | agcagcacggggccgtcgccgatgggggtgttctgctggtagtggtcggcgagctgcacgctgccgt |
| | cctcgatgttgtggcggatcttgaagttcaccttgatgccgttcttctgcttgtcggccatgatata |
| | gacgttgtggctgttgtagttgtactccagcttgtgccccaggatgttgccgtcctccttgaagtcg |
| | atgcccttcagctcgatgcggttcaccagggtgtcgccctcgaacttcacctcggcgcgggtcttgt |
| | agttgccgtcgtccttgaagaagatggtgcgctcctggacgtagccttcgggcatggcggacttgaa |
| | gaagtcgtgctgcttcatgtggtcggggtagcggctgaagcactgcacgccgtaggtcagggtggtc |
| | acgagggtgggccagggcacgggcagcttgccggtggtgcagatgaacttcagggtcagcttgccgt |
| | aggtggcatcgccctcgccctcgccggacacgctgaacttgtggccgtttacgtcgccgtccagctc |
| | gaccaggatgggcaccaccccggtgaacagctcctcgcccttgctcaccatggtggcgggatctgac |
| | ggttcactaaaccagctctgcttatatagacctcccaccgtacacgcctaccgcccatttgcgtcaa |
| | tggggcggagttgttacgacattttggaaagtcccgttgattttggtgccaaaacaaactcccattg |
| | acgtcaatggggtggagacttggaaatccccgtgagtcaaaccgctatccacgcccattgatgtact |
| | gccaaaaccgcatcaccatggtaatagcgatgactaatacgtagatgtactgccaagtaggaaagtc |
| | ccataaggtcatgtactgggcataatgccaggcgggccatttaccgtcattgacgtcaatagggggc |
| | gtacttggcatatgatacacttgatgtactgccaagtgggcagtttaccgtaaatactccacccatt |
| | gacgtcaatggaaagtccctattggcgttactatgggaacatacgtcattattgacgtcaatgggcg |
| | ggggtcgttgggcggtcagccaggcgggccatttaccgtaagttatgtaacgGGCCTGCTGCCGGCT |
| | CTGCGGCCTCTTCCGCGTCTTCGCCTTCGCCCTCAGACGAGTCGGATCTCCCTTTGGGCCGCCTCCC |
| | CGCCTGTCTAGCTTGACTGACTGAGATACAGCGTACCTTCAGCTCACAGACATGATAAGATACATTG |
| | ATGAGTTTGGACAAACCACAACTAGAATGCAGTGAAAAAAATGCTTTATTTGTGAAATTTGTGATGC |
| | TATTGCTTTATTTGTAACCATTATAAGCTGCAATAAACAAGTTAACAACAACAATTGCATTCATTTT |
| | ATGTTTCAGGTTCAGGGGGAGGTGTGGGAGGTTTTTTAAAGCAAGTAAAACCTCTACAAATGTGGTA |
| | TTGGCCCATCTCTATCGGTATCGTAGCATAACCCCTTGGGGCCTCTAAACGGGTCTTGAGGGGTTTT |
| | TTGTGCCCCTCGGGCCGGATTGCTATCTACCGGCATTGGCGCAGAAAAAAATGCCTGATGCGACGCT |
| | GCGCGTCTTATACTCCCACATATGCCAGATTCAGCAACGGATACGGCTTCCCCAACTTGCCCACTTC |
| | CATACGTGTCCTCCTTACCAGAAATTTATCCTTAAGGTCGTCAGCTATCCTGCAGGCGATCTCTCGA |
| | TTTCGATCAAGACATTCCTTTAATGGTCTTTTCTGGACACCACTAGGGGTCAGAAGTAGTTCATCAA |
| | ACTTTCTTCCCTCCCTAATCTCATTGGTTACCTTGGGCTATCGAAACTTAATTAAGCGATCTGCATC |
| | TCAATTAGTCAGCAACCATAGTCCCGCCCCTAACTCCGCCCATCCCGCCCCTAACTCCGCCCAGTTC |
| | CGCCCATTCTCCGCCCCATCGCTGACTAATTTTTTTTATTTATGCAGAGGCCGAGGCCGCCTCGGCC |
| | TCTGAGCTATTCCAGAAGTAGTGAGGAGGCTTTTTTGGAGGCCTAGGCTTTTGCAAAGGAGGTAGCC |
| | AACATGATTGAACAAGATGGATTGCACGCAGGTTCTCCCGCCGCTTGGGTGGAGAGGCTATTCGGCT |
| | ATGACTGGGCACAACAGACAATCGGCTGCTCTGATGCCGCCGTGTTCCGGCTGTCAGCGCAGGGGCG |
| | CCCGGTTCTTTTTGTCAAGACCGACCTGTCCGGTGCCCTGAATGAACTCCAGGACGAGGCAGCGCGG |
| | CTATCGTGGCTGGCCACGACGGGCGTTCCTTGCGCAGCTGTGCTCGACGTTGTCACTGAAGCGGGAA |
| | GGGACTGGCTGCTATTGGGCGAAGTGCCGGGGCAGGATCTCCTGTCATCTCACCTTGCTCCTGCCGA |
| | GAAAGTATCCATCATGGCTGATGCAATGCGGCGGCTGCATACGCTTGATCCGGCTACCTGCCCATTC |
| | GACCACCAAGCGAAACATCGCATCGAGCGAGCACGTACTCGGATGGAAGCCGGTCTTGTCGATCAGG |
| | ATGATCTGGACGAAGAGCATCAGGGGCTCGCGCCAGCCGAACTGTTCGCCAGGCTCAAGGCGCGGAT |
| | GCCCGACGGCGAGGATCTCGTCGTGACCCACGGCGATGCCTGCTTGCCGAATATCATGGTGGAAAAT |
| | GGCCGCTTTTCTGGATTCATCGACTGTGGCCGGCTGGGTGTGGCGGACCGCTATCAGGACATAGCGT |
| | TGGCTACCCGTGATATTGCTGAAGAGCTTGGCGGCGAATGGGCTGACCGCTTCCTCGTGCTTTACGG |
| | TATCGCCGCTCCCGATTCGCAGCGCATCGCCTTCTATCGCCTTCTTGACGAGTTCTTCTAGTATGTA |
| | AGCCCTGTGCCTTCTAGTTGCCAGCCATCTGTTGTTTGCCCCTCCCCCGTGCCTTCCTTGACCCTGG |
| | AAGGTGCCACTCCCACTGTCCTTTCCTAATAAAATGAGGAAATTGCATCGCATTGTCTGAGTAGGTG |
| | TCATTCTATTCTGGGGGGTGGGGTGGGGCAGGACAGCAAGGGGGAGGATTGGGAAGACAATAGCAGG |
| | CATGCTGGGGATGCGGTGGGCTCTATGGTTAATTAACCAGTCAAGTCAGCTACTTGGCGAGATCGAC |
| | TTGTCTGGGTTTCGACTACGCTCAGAATTGCGTCAGTCAAGTTCGATCTGGTCCTTGCTATTGCACC |
| | CGTTCTCCGATTACGAGTTTCATTTAAATCATGTGAGCAAAAGGCCAGCAAAAGGCCAGGAACCGTA |
| | AAAAGGCCGCGTTGCTGGCGTTTTTCCATAGGCTCCGCCCCCCTGACGAGCATCACAAAAATCGACG |
| | CTCAAGTCAGAGGTGGCGAAACCCGACAGGACTATAAAGATACCAGGCGTTTCCCCCTGGAAGCTCC |
| | CTCGTGCGCTCTCCTGTTCCGACCCTGCCGCTTACCGGATACCTGTCCGCCTTTCTCCCTTCGGGAA |
| | GCGTGGCGCTTTCTCATAGCTCACGCTGTAGGTATCTCAGTTCGGTGTAGGTCGTTCGCTCCAAGCT |
| | GGGCTGTGTGCACGAACCCCCCGTTCAGCCCGACCGCTGCGCCTTATCCGGTAACTATCGTCTTGAG |
| | TCCAACCCGGTAAGACACGACTTATCGCCACTGGCAGCAGCCACTGGTAACAGGATTAGCAGAGCGA |
| | GGTATGTAGGCGGTGCTACAGAGTTCTTGAAGTGGTGGCCTAACTACGGCTACACTAGAAGAACAGT |
| | ATTTGGTATCTGCGCTCTGCTGAAGCCAGTTACCTTCGGAAAAAGAGTTGGTAGCTCTTGATCCGGC |
| | AAACAAACCACCGCTGGTAGCGGTGGTTTTTTTGTTTGCAAGCAGCAGATTACGCGCAGAAAAAAAG |
| | GATCTCAAGAAGATCCTTTGATCTTTTCTACGGGGTCTGACGCTCAGTGGAACGAAAACTCACGTTA |
| | AGGGATTTTGGTCATGAGATTATCAAAAAGGATCTTCACCTAGATCCTTTTAAATTAAAAATGAAGT |
| | TTTAAATCAATCTAAAGTATATATGAGTAAACTTGGTCTGACAGTTACCAATGCTTAATCAGTGAGG |
| | CACCTATCTCAGCGATCTGTCTATTTCGTTCATCCATAGTTGCATTTAAATTTCCGAACTCTCCAAG |
| | GCCCTCGTCGGAAAATCTTCAAACCTTTCGTCCGATCCATCTTGCAGGCTACCTCTCGAACGAACTA |
| | TCGCAAGTCTCT (SEQ ID NO: 109) |
| ret-029- | ACCGAGAACGATCCTCTCAGTGCGAGTCTCGACGATCCATATCGTTGCTTGGCAGTCAGCCAGTCGG |
| line1- | AATCCAGCTTGGGACCCAGGAAGTCCAATCGTCAGATATTGTACTCAAGCCTGGTCACGGCAGCGTA |
| orf1-t2a- | CCGATCTGTTTAAACCTAGATATTGATAGTCTGATCGGTCAACGTATAATCGAGTCCTAGCTTTTGC |
| orf- | AAACATCTATCAAGAGACAGGATCAGCAGGAGGCTTTCGCATGAGTATTCAACATTTCCGTGTCGCC |
| c_nucleo- | CTTATTCCCTTTTTTGCGGCATTTTGCCTTCCTGTTTTTGCTCACCCAGAAACGCTGGTGAAAGTAA |
| plasmin_ | AAGATGCTGAAGATCAGTTGGGTGCGCGAGTGGGTTACATCGAACTGGATCTCAACAGCGGTAAGAT |
| nls-gfp | CCTTGAGAGTTTTCGCCCCGAAGAACGCTTTCCAATGATGAGCACTTTTAAAGTTCTGCTATGTGGC |
| | GCGGTATTATCCCGTATTGACGCCGGGCAAGAGCAACTCGGTCGCCGCATACACTATTCTCAGAATG |
| | ACTTGGTTGAGTATTCACCAGTCACAGAAAAGCATCTTACGGATGGCATGACAGTAAGAGAATTATG |
| | CAGTGCTGCCATAACCATGAGTGATAACACTGCGGCCAACTTACTTCTGACAACGATTGGAGGACCG |
| | AAGGAGCTAACCGCTTTTTTGCACAACATGGGGGATCATGTAACTCGCCTTGATCGTTGGGAACCGG |
| | AGCTGAATGAAGCCATACCAAACGACGAGCGTGACACCACGATGCCTGTAGCAATGGCAACAACCTT |
| | GCGTAAACTATTAACTGGCGAACTACTTACTCTAGCTTCCCGGCAACAGTTGATAGACTGGATGGAG |
| | GCGGATAAAGTTGCAGGACCACTTCTGCGCTCGGCCCTTCCGGCTGGCTGGTTTATTGCTGATAAAT |
| | CTGGAGCCGGTGAGCGTGGGTCTCGCGGTATCATTGCAGCACTGGGGCCAGATGGTAAGCCCTCCCG |
| | TATCGTAGTTATCTACACGACGGGGAGTCAGGCAACTATGGATGAACGAAATAGACAGATCGCTGAG |
| | ATAGGTGCCTCACTGATTAAGCATTGGTAACCGATTCTAGGTGCATTGGCGCAGAAAAAAATGCCTG |
| | ATGCGACGCTGCGCGTCTTATACTCCCACATATGCCAGATTCAGCAACGGATACGGCTTCCCCAACT |
| | TGCCCACTTCCATACGTGTCCTCCTTACCAGAAATTTATCCTTAAGATCGTTTAAACTCGACTCTGG |
| | CTCTATCGAATCTCCGTCGTTTCGAGCTTACGCGAACAGCCGTGGCGCTCATTTGCTCGTCGGGCAT |
| | CGAATCTCGTCAGCTATCGTCAGCTTACCTTTTTGGCAGCGATCGCGGCTCCCGACATCTTGGACCA |
| | TTAGCTCCACAGGTATCTTCTTCCCTCTAGTGGTCATAACAGCAGCTTCAGCTACCTCTCAATTCAA |
| | AAAACCCCTCAAGACCCGTTTAGAGGCCCCAAGGGGTTATGCTATCAATCGTTGCGTTACACACACA |
| | AAAAACCAACACACATCCATCTTCGATGGATAGCGATTTTATTATCTAACTGCTGATCGAGTGTAGC |
| | CAGATCTAGTAATCAATTACGGGGTCATTAGTTCATAGCCCATATATGGAGTTCCGCGTTACATAAC |
| | TTACGGTAAATGGCCCGCCTGGCTGACCGCCCAACGACCCCCGCCCATTGACGTCAATAATGACGTA |
| | TGTTCCCATAGTAACGCCAATAGGGACTTTCCATTGACGTCAATGGGTGGAGTATTTACGGTAAACT |
| | GCCCACTTGGCAGTACATCAAGTGTATCATATGCCAAGTACGCCCCCTATTGACGTCAATGACGGTA |
| | AATGGCCCGCCTGGCATTATGCCCAGTACATGACCTTATGGGACTTTCCTACTTGGCAGTACATCTA |
| | CGTATTAGTCATCGCTATTACCATGCTGATGCGGTTTTGGCAGTACATCAATGGGCGTGGATAGCGG |
| | TTTGACTCACGGGGATTTCCAAGTCTCCACCCCATTGACGTCAATGGGAGTTTGTTTTGGCACCAAA |
| | ATCAACGGGACTTTCCAAAATGTCGTAACAACTCCGCCCCATTGACGCAAATGGGCGGTAGGCGTGT |
| | ACGGTGGGAGGTCTATATAAGCAGAGCTGGTTTAGTGAACCGTCAGATCAGATCTTTGTCGATCCTA |
| | CCATCCACTCGACACACCCGCCAGCGGCCGCTAATACGACTCACTATAGGGAGAAGTACTGCCACCA |
| | TGGGCAAGAAGCAAAATCGCAAGACGGGGAATTCCAAGACACAATCCGCTAGCCCACCACCTAAAGA |
| | GCGTTCTAGCTCCCCTGCTACTGAGCAGTCCTGGATGGAAAACGACTTCGATGAACTCCGGGAAGAG |
| | GGATTTAGGCGATCCAACTATTCAGAACTCCGCGAAGATATCCAGACAAAGGGGAAGGAAGTCGAGA |
| | ATTTCGAGAAGAACCTCGAGGAGTGCATCACCCGTATCACAAACACTGAGAAATGTCTCAAAGAACT |
| | CATGGAACTTAAGACAAAAGCCAGGGAGCTTCGAGAGGAGTGTCGGAGTCTGAGATCCAGGTGTGAC |
| | CAGCTCGAGGAGCGCGTGAGCGCGATGGAAGACGAGATGAACGAGATGAAAAGAGAGGGCAAATTCA |
| | GGGAGAAGCGCATTAAGAGGAACGAACAGAGTCTGCAGGAGATTTGGGATTACGTCAAGAGGCCTAA |
| | CCTGCGGTTGATCGGCGTCCCCGAGAGCGACGTAGAAAACGGGACTAAACTGGAGAATACACTTCAA |
| | GACATCATTCAAGAAAATTTTCCAAACCTGGCTCGGCAAGCTAATGTGCAAATCCAAGAGATCCAAC |
| | GCACACCCCAGCGGTATAGCTCTCGGCGTGCCACCCCTAGGCATATTATCGTGCGCTTTACTAAGGT |
| | GGAGATGAAAGAGAAGATGCTGCGAGCCGCTCGGGAAAAGGGAAGGGTGACTTTGAAGGGCAAACCT |
| | ATTCGGCTGACGGTTGACCTTAGCGCCGAGACACTCCAGGCACGCCGGGAATGGGGCCCCATCTTTA |
| | ATATCCTGAAGGAGAAGAACTTCCAGCCACGAATCTCTTACCCTGCAAAGTTGAGTTTTATCTCCGA |
| | GGGTGAGATTAAGTATTTCATCGATAAACAGATGCTGCGAGACTTCGTGACAACTCGCCCAGCTCTC |
| | AAGGAACTGCTCAAAGAGGCTCTTAATATGGAGCGCAATAATAGATATCAACCCTTGCAGAACCACG |
| | CAAAGATGggctccggcgagggcaggggaagCcttctaacatgcggggacgtggaggaaaatcccgg |
| | cccaGGTAGCGGCACCGGCTCTAACTCACATATCACCATCCTTACACTTAACATTAACGGCCTCAAC |
| | TCAGCTATCAAGCGCCATCGGCTGGCCAGCTGGATCAAATCACAGGATCCAAGCGTTTGTTGCATCC |
| | AAGAGACCCACCTGACCTGTAGAGATACTCACCGCCTCAAGATCAAGGGATGGCGAAAGATTTATCA |
| | GGCGAACGGTAAGCAGAAGAAAGCCGGAGTCGCAATTCTGGTCTCAGACAAGACGGATTTCAAGCCC |
| | ACCAAAATTAAGCGTGATAAGGAAGGTCACTATATTATGGTGAAAGGCAGCATACAGCAGGAAGAAC |
| | TTACCATATTGAACATCTACGCGCCAAACACCGGCGCACCTCGCTTTATCAAACAGGTCCTGTCCGA |
| | TCTGCAGCGAGATCTGGATTCTCATACGTTGATTATGGGTGATTTCAATACACCATTGAGCACCCTG |
| | GATCGCAGCACCAGGCAAAAGGTAAATAAAGACACGCAAGAGCTCAATAGCGCACTGCATCAGGCAG |
| | ATCTCATTGATATTTATCGCACTCTTCATCCTAAGAGTACCGAGTACACATTCTTCAGCGCCCCACA |
| | TCATACATACTCAAAGATCGATCATATCGTCGGCTCAAAGGCTCTGCTGTCAAAGTGCAAGCGCACA |
| | GAGATAATTACAAATTACCTGTCAGATCATAGCGCGATCAAGCTCGAGCTGAGAATCAAGAACCTGA |
| | CCCAGAGCCGGAGTACCACTTGGAAGCTTAATAACCTGCTGCTCAACGATTATTGGGTCCACAATGA |
| | GATGAAGGCAGAGATTAAAATGTTCTTCGAAACAAATGAGAATAAGGATACTACCTATCAAAACCTT |
| | TGGGATGCCTTTAAGGCCGTCTGCAGAGGCAAGTTCATCGCCCTCAACGCCTATAAAAGAAAACAAG |
| | AGAGATCTAAGATCGATACTCTCACCTCTCAGCTGAAGGAGTTGGAGAAACAGGAACAGACCCACTC |
| | CAAGGCGTCAAGACGGCAGGAGATCACAAAGATTCGCGCCGAGTTGAAAGAGATCGAAACCCAAAAG |
| | ACTCTTCAGAAAATTAACGAGTCTCGTAGTTGGTTCTTCGAGCGGATTAATAAGATAGACAGACCTC |
| | TGGCACGACTGATTAAGAAGAAGCGCGAAAAGAACCAGATTGATACCATCAAGAACGACAAGGGCGA |
| | CATCACTACTGACCCGACCGAGATCCAGACCACTATTCGGGAGTATTATAAGCATTTGTATGCTAAC |
| | AAGCTTGAGAACCTGGAAGAGATGGACACTTTTCTGGATACCTATACTCTGCCACGGCTTAATCAAG |
| | AGGAAGTCGAGTCCCTCAACCGCCCAATTACAGGAAGCGAGATTGTGGCCATAATTAACTCCCTGCC |
| | GACAAAGAAATCTCCTGGTCCGGACGGGTTTACAGCTGAGTTTTATCAACGGTATATGGAAGAGCTT |
| | GTACCGTTTCTGCTCAAGCTCTTTCAGTCTATAGAAAAGGAAGGCATCTTGCCCAATTCCTTCTACG |
| | AAGCTTCTATAATACTTATTCCCAAACCAGGACGCGATACCACAAAGAAGGAAAACTTCCGGCCCAT |
| | TAGTCTCATGAATATCGACGCTAAAATATTGAACAAGATTCTCGCCAACAGAATCCAACAACATATT |
| | AAGAAATTGATACATCACGACCAGGTGGGGTTTATACCTGGCATGCAGGGCTGGTTTAACATCCGGA |
| | AGAGTATTAACGTCATTCAACACATTAATAGAGCTAAGGATAAGAATCATATGATCATCTCTATAGA |
| | CGCGGAAAAGGCATTCGATAAGATTCAGCAGCCATTTATGCTCAAGACTCTGAACAAACTCGGCATC |
| | GACGGAACATATTTTAAGATTATTCGCGCAATTTACGATAAGCCGACTGCTAACATTATCCTTAACG |
| | GCCAAAAGCTCGAGGCCTTTCCGCTCAAGACTGGAACCCGCCAAGGCTGTCCCCTCTCCCCGCTTTT |
| | GTTTAATATTGTACTCGAGGTGCTGGCTAGGGCTATTCGTCAAGAGAAAGAGATTAAAGGGATACAG |
| | CTCGGGAAGGAAGAGGTCAAGCTTTCCTTGTTCGCCGATGATATGATTGTGTACCTGGAGAATCCTA |
| | TTGTGTCTGCTCAGAACCTTCTTAAACTTATTTCTAACTTTAGCAAGGTCAGCGGCTATAAGATTAA |
| | CGTCCAGAAATCTCAGGCCTTTCTGTACACAAATAATCGACAGACCGAATCCCAGATAATGGGTGAG |
| | CTTCCGTTTGTCATAGCCAGCAAAAGGATAAAGTATCTCGGAATCCAGCTGACACGAGACGTTAAAG |
| | ATTTGTTTAAGGAAAATTACAAGCCTCTCCTGAAAGAGATTAAGGAAGATACTAATAAGTGGAAGAA |
| | TATCCCCTGTTCATGGGTTGGCAGAATCAACATAGTGAAGATGGCAATACTTCCTAAAGTGATATAT |
| | CGCTTTAACGCCATCCCAATTAAACTGCCTATGACCTTCTTTACGGAGCTCGAGAAAACAACCCTTA |
| | AATTTATATGGAATCAAAAGAGAGCAAGAATAGCGAAGTCCATCTTGAGCCAGAAGAATAAGGCCGG |
| | TGGGATTACTTTGCCTGATTTTAAGTTGTATTATAAAGCCACAGTAACTAAGACAGCCTGGTATTGG |
| | TATCAGAATAGAGACATCGACCAGTGGAATCGGACCGAACCATCAGAGATAATGCCCCACATCTATA |
| | ATTACCTTATATTCGATAAGCCAGAAAAGAATAAACAGTGGGGCAAAGACAGCCTCTTCAACAAGTG |
| | GTGTTGGGAGAATTGGCTGGCCATATGCCGGAAACTCAAGCTCGACCCCTTTCTTACACCCTACACT |
| | AAAATCAACAGTAGGTGGATCAAGGACTTGAATGTCAAGCCAAAGACTATAAAGACACTGGAAGAGA |
| | ATCTTGGGATCACAATACAAGATATAGGCGTCGGCAAAGATTTTATGTCAAAGACGCCCAAGGCCAT |
| | GGCCACTAAGGATAAGATTGATAAGTGGGACCTTATTAAGCTCAAAAGCTTCTGTACTGCCAAGGAG |
| | ACCACGATCAGAGTTAATAGGCAGCCCACTACATGGGAAAAGATTTTCGCCACTTATTCATCAGATA |
| | AGGGGTTGATAAGCAGAATATATAACGAGCTGAAGCAGATCTACAAGAAGAAAACGAATAATCCCAT |
| | CAAGAAGTGGGCAAAAGATATGAACAGGCATTTTAGCAAAGAGGATATCTACGCCGCGAAGAAGCAT |
| | ATGAAGAAGTGTAGTTCAAGCTTGGCCATTCGTGAGATGCAGATTAAGACGACCATGCGATACCACC |
| | TTACCCCAGTGAGGATGGCAATTATCAAGAAATCTGGCAATAATAGATGTTGGCGGGGCTGTGGCGA |
| | GATTGGCACCCTGCTCCATTGCTGGTGGGATTGCAAGCTGGTGCAGCCGCTTTGGAAATCAGTCTGG |
| | CGCTTTCTGAGGGACCTCGAGCTTGAGATTCCCTTCGATCCCGCAATTCCCTTGCTCGGAATCTATC |
| | CTAACGAATACAAGAGCTGTTGTTACAAGGATACGTGTACCCGGATGTTCATCGCGGCCTTGTTTAC |
| | GATAGCTAAGACGTGGAATCAGCCTAAGTGCCCCACAATGATCGATTGGATCAAGAAAATGTGGCAT |
| | ATTTATACCATGGAGTATTACGCAGCAATTAAGAATGACGAATTTATTTCCTTCGTTGGGACCTGGA |
| | TGAAGCTGGAGACTATTATTCTGAGCAAGCTGTCTCAGGAGCAAAAGACAAAGCATAGAATCTTCTC |
| | TCTCATTGGTGGTAACGACTACAAAGACGATGACGACAAGaaaaggccggcggccacgaaaaaggcc |
| | ggccaggcaaaaaagaaaaagTAAAGCGCTTCTAGAAGTTGTCTCCTCCTGCACTGACTGACTGATA |
| | CAATCGATTTCTGGATCCGCAGGCCTAATCAACCTCTGGATTACAAAATTTGTGAAAGATTGACTGG |
| | TATTCTTAACTATGTTGCTCCTTTTACGCTATGTGGATACGCTGCTTTAATGCCTTTGTATCATGCT |
| | ATTGCTTCCCGTATGGCTTTCATTTTCTCCTCCTTGTATAAATCCTGGTTGCTGTCTCTTTATGAGG |
| | AGTTGTGGCCCGTTGTCAGGCAACGTGGCGTGGTGTGCACTGTGTTTGCTGACGCAACCCCCACTGG |
| | TTGGGGCATTGCCACCACCTGTCAGCTCCTTTCCGGGACTTTCGCTTTCCCCCTCCCTATTGCCACG |
| | GCGGAACTCATCGCCGCCTGCCTTGCCCGCTGCTGGACAGGGGCTCGGCTGTTGGGCACTGACAATT |
| | CCGTGGTGTTGTCGGGGAAGCTGACGTCCTTTCCATGGCTGCTCGCCTGTGTTGCCACCTGGATTCT |
| | GCGCGGGACGTCCTTCTGCTACGTCCCTTCGGCCCTCAATCCAGCGGACCTTCCTTCCCGCtgagag |
| | acacaaaaaattccaacacactattgcaatgaaaataaatttcctttattagccagaagtcagatgc |
| | tcaaggggcttcatgatgtccccataatttttggcagagggaaaaagatctcagtggtatttgtgag |
| | ccagggcattggccttctgataggcagcctgcacctgaggagtgcggccgctttacttgtacagctc |
| | gtccatgccgagagtgatcccggcggcggtcacgaactccagcaggaccatgtgatcgcgcttctcg |
| | ttggggtctttgctcagggcggactgggtgctcaggtagtggttgtcgggcagcagcacggggccgt |
| | cgccgatgggggtgttctgctggtagtggtcggcgagctgcacgctgccgtcctcgatgttgtggcg |
| | gatcttgaagttcaccttgatgccgttcttctgcttgtcggccatgatatagacgttgtggctgttg |
| | tagttgtactccagcttgtgccccaggatgttgccgtcctccttgaagtcgatgcccttcagctcga |
| | tgcggttcaccagggtgtcgccctcgaacttcacctcggcgcgggtcttgtagttgccgtcgtcctt |
| | gaagaagatggtgcgctcctggacgtagccttcgggcatggcggacttgaagaagtcgtgctgcttc |
| | atgtggtcggggtagcggctgaagcactgcacgccgtaggtcagggtggtcacgagggtgggccagg |
| | gcacgggcagcttgccggtggtgcagatgaacttcagggtcagcttgccgtaggtggcatcgccctc |
| | gccctcgccggacacgctgaacttgtggccgtttacgtcgccgtccagctcgaccaggatgggcacc |
| | accccggtgaacagctcctcgcccttgctcaccatggtggcgggatctgacggttcactaaaccagc |
| | tctgcttatatagacctcccaccgtacacgcctaccgcccatttgcgtcaatggggcggagttgtta |
| | cgacattttggaaagtcccgttgattttggtgccaaaacaaactcccattgacgtcaatggggtgga |
| | gacttggaaatccccgtgagtcaaaccgctatccacgcccattgatgtactgccaaaaccgcatcac |
| | catggtaatagcgatgactaatacgtagatgtactgccaagtaggaaagtcccataaggtcatgtac |
| | tgggcataatgccaggcgggccatttaccgtcattgacgtcaatagggggcgtacttggcatatgat |
| | acacttgatgtactgccaagtgggcagtttaccgtaaatactccacccattgacgtcaatggaaagt |
| | ccctattggcgttactatgggaacatacgtcattattgacgtcaatgggcgggggtcgttgggcggt |
| | cagccaggcgggccatttaccgtaagttatgtaacgGGCCTGCTGCCGGCTCTGCGGCCTCTTCCGC |
| | GTCTTCGCCTTCGCCCTCAGACGAGTCGGATCTCCCTTTGGGCCGCCTCCCCGCCTGTCTAGCTTGA |
| | CTGACTGAGATACAGCGTACCTTCAGCTCACAGACATGATAAGATACATTGATGAGTTTGGACAAAC |
| | CACAACTAGAATGCAGTGAAAAAAATGCTTTATTTGTGAAATTTGTGATGCTATTGCTTTATTTGTA |
| | ACCATTATAAGCTGCAATAAACAAGTTAACAACAACAATTGCATTCATTTTATGTTTCAGGTTCAGG |
| | GGGAGGTGTGGGAGGTTTTTTAAAGCAAGTAAAACCTCTACAAATGTGGTATTGGCCCATCTCTATC |
| | GGTATCGTAGCATAACCCCTTGGGGCCTCTAAACGGGTCTTGAGGGGTTTTTTGTGCCCCTCGGGCC |
| | GGATTGCTATCTACCGGCATTGGCGCAGAAAAAAATGCCTGATGCGACGCTGCGCGTCTTATACTCC |
| | CACATATGCCAGATTCAGCAACGGATACGGCTTCCCCAACTTGCCCACTTCCATACGTGTCCTCCTT |
| | ACCAGAAATTTATCCTTAAGGTCGTCAGCTATCCTGCAGGCGATCTCTCGATTTCGATCAAGACATT |
| | CCTTTAATGGTCTTTTCTGGACACCACTAGGGGTCAGAAGTAGTTCATCAAACTTTCTTCCCTCCCT |
| | AATCTCATTGGTTACCTTGGGCTATCGAAACTTAATTAAGCGATCTGCATCTCAATTAGTCAGCAAC |
| | CATAGTCCCGCCCCTAACTCCGCCCATCCCGCCCCTAACTCCGCCCAGTTCCGCCCATTCTCCGCCC |
| | CATCGCTGACTAATTTTTTTTATTTATGCAGAGGCCGAGGCCGCCTCGGCCTCTGAGCTATTCCAGA |
| | AGTAGTGAGGAGGCTTTTTTGGAGGCCTAGGCTTTTGCAAAGGAGGTAGCCAACATGATTGAACAAG |
| | ATGGATTGCACGCAGGTTCTCCCGCCGCTTGGGTGGAGAGGCTATTCGGCTATGACTGGGCACAACA |
| | GACAATCGGCTGCTCTGATGCCGCCGTGTTCCGGCTGTCAGCGCAGGGGCGCCCGGTTCTTTTTGTC |
| | AAGACCGACCTGTCCGGTGCCCTGAATGAACTCCAGGACGAGGCAGCGCGGCTATCGTGGCTGGCCA |
| | CGACGGGCGTTCCTTGCGCAGCTGTGCTCGACGTTGTCACTGAAGCGGGAAGGGACTGGCTGCTATT |
| | GGGCGAAGTGCCGGGGCAGGATCTCCTGTCATCTCACCTTGCTCCTGCCGAGAAAGTATCCATCATG |
| | GCTGATGCAATGCGGCGGCTGCATACGCTTGATCCGGCTACCTGCCCATTCGACCACCAAGCGAAAC |
| | ATCGCATCGAGCGAGCACGTACTCGGATGGAAGCCGGTCTTGTCGATCAGGATGATCTGGACGAAGA |
| | GCATCAGGGGCTCGCGCCAGCCGAACTGTTCGCCAGGCTCAAGGCGCGGATGCCCGACGGCGAGGAT |
| | CTCGTCGTGACCCACGGCGATGCCTGCTTGCCGAATATCATGGTGGAAAATGGCCGCTTTTCTGGAT |
| | TCATCGACTGTGGCCGGCTGGGTGTGGCGGACCGCTATCAGGACATAGCGTTGGCTACCCGTGATAT |
| | TGCTGAAGAGCTTGGCGGCGAATGGGCTGACCGCTTCCTCGTGCTTTACGGTATCGCCGCTCCCGAT |
| | TCGCAGCGCATCGCCTTCTATCGCCTTCTTGACGAGTTCTTCTAGTATGTAAGCCCTGTGCCTTCTA |
| | GTTGCCAGCCATCTGTTGTTTGCCCCTCCCCCGTGCCTTCCTTGACCCTGGAAGGTGCCACTCCCAC |
| | TGTCCTTTCCTAATAAAATGAGGAAATTGCATCGCATTGTCTGAGTAGGTGTCATTCTATTCTGGGG |
| | GGTGGGGTGGGGCAGGACAGCAAGGGGGAGGATTGGGAAGACAATAGCAGGCATGCTGGGGATGCGG |
| | TGGGCTCTATGGTTAATTAACCAGTCAAGTCAGCTACTTGGCGAGATCGACTTGTCTGGGTTTCGAC |
| | TACGCTCAGAATTGCGTCAGTCAAGTTCGATCTGGTCCTTGCTATTGCACCCGTTCTCCGATTACGA |
| | GTTTCATTTAAATCATGTGAGCAAAAGGCCAGCAAAAGGCCAGGAACCGTAAAAAGGCCGCGTTGCT |
| | GGCGTTTTTCCATAGGCTCCGCCCCCCTGACGAGCATCACAAAAATCGACGCTCAAGTCAGAGGTGG |
| | CGAAACCCGACAGGACTATAAAGATACCAGGCGTTTCCCCCTGGAAGCTCCCTCGTGCGCTCTCCTG |
| | TTCCGACCCTGCCGCTTACCGGATACCTGTCCGCCTTTCTCCCTTCGGGAAGCGTGGCGCTTTCTCA |
| | TAGCTCACGCTGTAGGTATCTCAGTTCGGTGTAGGTCGTTCGCTCCAAGCTGGGCTGTGTGCACGAA |
| | CCCCCCGTTCAGCCCGACCGCTGCGCCTTATCCGGTAACTATCGTCTTGAGTCCAACCCGGTAAGAC |
| | ACGACTTATCGCCACTGGCAGCAGCCACTGGTAACAGGATTAGCAGAGCGAGGTATGTAGGCGGTGC |
| | TACAGAGTTCTTGAAGTGGTGGCCTAACTACGGCTACACTAGAAGAACAGTATTTGGTATCTGCGCT |
| | CTGCTGAAGCCAGTTACCTTCGGAAAAAGAGTTGGTAGCTCTTGATCCGGCAAACAAACCACCGCTG |
| | GTAGCGGTGGTTTTTTTGTTTGCAAGCAGCAGATTACGCGCAGAAAAAAAGGATCTCAAGAAGATCC |
| | TTTGATCTTTTCTACGGGGTCTGACGCTCAGTGGAACGAAAACTCACGTTAAGGGATTTTGGTCATG |
| | AGATTATCAAAAAGGATCTTCACCTAGATCCTTTTAAATTAAAAATGAAGTTTTAAATCAATCTAAA |
| | GTATATATGAGTAAACTTGGTCTGACAGTTACCAATGCTTAATCAGTGAGGCACCTATCTCAGCGAT |
| | CTGTCTATTTCGTTCATCCATAGTTGCATTTAAATTTCCGAACTCTCCAAGGCCCTCGTCGGAAAAT |
| | CTTCAAACCTTTCGTCCGATCCATCTTGCAGGCTACCTCTCGAACGAACTATCGCAAGTCTCTTGGC |
| | CGGCCTTGCGCCTTGGCTATTGCTTGGCAGCGCCTATCGCCAGGTATTACTCCAATCCCGAATATCC |
| | GAGATCGGGATCACCCGAGAGAAGTTCAACCTACATCCTCAATCCCGATCTATCCGAGATCCGAGGA |
| | ATATCGAAATCGGGGCGCGCCTGGTGT (SEQ ID NO: 110) |

### Example 22. Effects of Introducing a Nuclear Export Signal on Retrotransposition by Human LINE1 Constructs

In this example, addition of a nuclear export sequence is tested for improving translation of the cargo sequence. Addition of an NES at the C terminal or N terminal of the GFP is tested for increase in expression. Addition of multiple NESs at the C terminal or N terminal of the GFP is tested for increase in expression. Larger cargo (larger than 5 kB coding sequence, or larger than 7.5 kB coding sequence), will be tested to see if NES has effect on cargo of certain sizes. Additional cargo may be tested that specifically require trans-golgi or ER localization, e.g., cargo having a transmembrane domain.

### Example 23. Screen for Efficient LINE1 System for Retrotransposition of Various Human Cells

In this example, a phylogenetic screen is undertaken across a large number of organisms using bioinformatics followed by laboratory testing, for selecting one or more hyperactive retrotransposon element that can efficiently move within human genome. Over 10 million LINE-1 sequences from 503 different genomes were identified (Ivancevic et al., 2016), including ORF1 and ORF2 proteins with novel domain variations. Among these, the ‘hyperactive’LINE-1 species were shown to display retrotransposition activity superior to that of human, rat and mouse. These elements will be cloned into LINE-1-GFPai plasmid and screened in HEK293T cells. Test mRNA constructs comprising a sequence encoding GFP along with a promoter and a poly A sequence, inserted in reverse orientation relative to the ORF1/2 genes in the retrotransposon complex as described elsewhere in the specification are used for electroporating the cells. Efficiency of integration is determined by flow cytometry and PCR. As above, GFP is measured in negative control and positive control to set gates for flow cytometry. As above, an exemplary negative control used will be set at <0.1% GFP+. As above, an exemplary positive control used will be set at >90% GFP+. Integration will be tested using PCR. An exemplary measurement indicative of successful integration is measurement of GFP expression, as determined by flow cytometry, in >10% of cells by day 10 post retrotransposition.

### Example 24. Improvement of Cargo Gene Expression

To improve the cargo gene expression, various cargo gene promoters and poly(A) signals will be tested. To improve the cargo gene expression, various insertions of a short constitutively spliced introns will also be tested. To improve the cargo gene expression, codon optimized sequences, such as those generated using various alternative codon algorithms, will also be tested. As above, GFP is measured in negative control and positive control to set gates for flow cytometry. As above, an exemplary negative control used will be set at <0.1% GFP+. As above, an exemplary positive control used will be set at >90% GFP+. An exemplary measurement indicative of successful cargo gene expression is measurement of increased GFP expression, as determined by flow cytometry, in >10% of cells by day 10 post retrotransposition.

### Example 25. Adapting Efficiency-Optimized Retrotransposon System for In Vivo Cargo Gene Delivery

The retrotransposon mRNA will be engineered for in vivo delivery using liver-targeting LNPs to detect an intracellular or secreted protein, in mice. Cell-type specific miRNA targets in the 3′ UTR of the cargo gene mRNA will be applied to target it for degradation in undesirable cell types. LNPs will be tested for efficient delivery in vivo. Mice will be administered the LNP encapsulated mRNA constructs that comprise a sequence encoding GFP along with a promoter and a poly A sequence flanking the GFP encoding sequence. The GFP gene is inserted in reverse orientation relative to the ORF1/2 genes in the retrotransposon complex as described elsewhere in the specification. Protein expression will be examined by imaging and test mice will be sacrificed and tissues harvested for histological analysis and PCR at determined time points.

Cell-specific targeting is achieved following systemic administration of the LNP encapsulated mRNA wherein the LNPs comprise surface modification to display antibodies or ligands that recognize cognate counterparts in the target cell.

Cell specific targeting is tested following designing specific constructs, wherein the UTRs are modified to encode one or more cell-type specific miRNA. When the recombinant mRNA is taken up by a cell that is not the intended target cell, miRNA present in the cell will bind to and destroy the mRNA.

Hence the mRNA can express in the intended cell type.

An exemplary measurement indicative of successful in vivo mRNA delivery to liver measurement of GFP expression, as determined by flow cytometry or histology in hepatocytes.

### Example 26. Targeting Retrotransposition to Specific Genomic Locations

In this example, retrotransposition design modifications are undertaken for increasing specificity of targeting at specific genomic locations. Retrotransposon fusion constructs are generated in which a targeting moiety is incorporated to ensure increase in targeting specificity of the ORF2p. One method is to retarget LINE1 elements with cargo to specifically integrate into one of the genomic safe harbor (GHS) sites with a high and sustainable transcriptional activity. AAVS1, CCR5 and ROSA26 are some of the GHS sites. As described above, specific LINE1 elements are selected after suitable screen for identifying an efficient LINE1 element that readily transposes in human. The selected LINE1 can be one that integrates into one of the GHS loci.

Another method is that the retrotransposon ORF2 is engineered bearing fusion of ORF2 with Cas9 and its mutants. Other candidates elements having a heterologous DNA binding domain with or without endonuclease activity are Cpf1, zinc finger element, TAL effector, Cas6-8 ‘cascade’, restriction endonuclease) which will either replace or complement the endonuclease domain of ORF2p.

The addition of homology arms of different length around the mRNA cargo will also be tested.

An exemplary construct is designed, comprising sequences encoding: ORF1; RT and dCas9; the cargo gene in antisense orientation, and a guide RNA to target site for priming RT activity. In one exemplary construct use of two guide RNA is tested, each fused to a dCas9 nickase, to determine if introducing nicks upstream and downstream of targeted locations improves site-specific integration and orientation of gene cargo.

Exemplary constructs are designed and tested in which the ORF2p lacks an EN (deleted), and is fused with a Cas 9 or its mutants, a Cas 12a (Cpf1), a Cas6-8 ‘cascade’, a Meganuclease, a Zinc-finger nuclease, a TALEN, or a restriction endonuclease, which directs the specificity and introduces the nicks on the genomic DNA.

Exemplary constructs are designed and tested in which the ORF2p comprises a mutant EN that has reduced or altered activity, and is fused with a Cas 9 or its mutants, a Cas 12a (Cpf1), a Cas6-8 ‘cascade’, a Meganuclease, a Zinc-finger nuclease, a TALEN, or a restriction endonuclease, which directs the specificity and introduces the nicks on the genomic DNA. For example, constructs are designed and tested in which retrotransposon plasmids will be engineered that encode a fusion of ORF2 protein with Cas9 and Cas9 mutants. mRNA containing ORF1, and mRNA encoding RT and dCas9 and the cargo gene in antisense orientation, and pegRNA will be delivered to target sites for priming RT activity. The use of two sgRNA, each fused to a dCas9 nickase, may be evaluated to determine if introducing nicks upstream and downstream of targeted locations improves site-specific integration and orientation of gene cargo. If modified LINE-1 RT fusion protein can bind its own mRNA and transport to nucleus, pegRNA could target genomic region and prime RT activity (via dCas9 nickase activity) so the LINE-1 RT can reverse transcribe mRNA gene cargo for integration into genomic DNA.

Efficiency of integration will be determined by flow cytometry. above, GFP is measured in negative control and positive control to set gates for flow cytometry. As above, an exemplary negative control used will be set at <0.1% GFP+. As above, an exemplary positive control used will be set at >90% GFP+. Integration will be confirmed by PCR. The sites of genomic integration are analyzed using NGS. Any construct with preferential integration in the specific genome location is subjected to several cycles of directed evolution to improve its efficiency and/or integration precision. An exemplary measurement indicative of successful gene integration and expression is 90% of cargo gene integrations detected in a specific genome location and GFP expression, as determined by flow cytometry, in >2% of cells by day 10 post retrotransposition.

What is claimed is:

1. A composition comprising one or more polynucleic acid molecules, wherein the one or more polynucleic acid molecules comprise:
(a) an RNA molecule comprising a sequence encoding a Cas nickase;
(b) a first guide RNA or a polynucleic acid molecule comprising a sequence encoding the first guide RNA, wherein the first guide RNA specifically binds a DNA sequence upstream of a target DNA sequence;
(c) a second guide RNA or a polynucleic acid molecule comprising a sequence encoding the second guide RNA, wherein the second guide RNA specifically binds to a DNA sequence downstream of the target DNA sequence;
(d) an RNA molecule comprising:
i. a sequence that encodes an exogenous therapeutic polypeptide or a sequence that is a reverse complement of the sequence that encodes the exogenous therapeutic polypeptide; and
ii. a mobile genetic element, wherein the mobile genetic element comprises an RNA sequence encoding a polypeptide with target-primed reverse transcription (TPRT) activity, wherein the polypeptide with TPRT activity comprises an endonuclease domain with a mutation that abrogates endonuclease activity of the endonuclease domain.

2. The composition of claim 1, wherein the one or more polynucleic acid molecules are encapsulated in a nanoparticle delivery vehicle.

3. The composition of claim 2, wherein the nanoparticle delivery vehicle is a lipid nanoparticle or a polymeric nanoparticle.

4. The composition of claim 1, wherein the polypeptide with TPRT activity is a human ORF2p polypeptide.

5. The composition of claim 4, wherein the human ORF2p polypeptide comprises an amino acid sequence having at least 80% sequence identity to SEQ ID NO: 59.

6. The composition of claim 5, wherein the human ORF2p polypeptide comprises an endonuclease domain with a mutation at D205 of SEQ ID NO: 59.

7. The composition of claim 1, wherein the polypeptide with TPRT activity comprises a nuclear localization signal (NLS).

8. The composition of claim 1, wherein the mobile genetic element comprises an RNA sequence encoding a human ORF1p polypeptide.

9. The composition of claim 8, wherein the ORF1p polypeptide comprises an amino acid sequence having at least 80% sequence identity to SEQ ID NO: 57.

10. The composition of claim 1, wherein the exogenous therapeutic polypeptide is selected from the group consisting of a ligand, an antibody, a receptor, an enzyme, a transport protein, a structural protein, a hormone, a contractile protein, a storage protein and a transcription factor.

11. The composition of claim 10, wherein the exogenous human therapeutic polypeptide is a receptor selected from the group consisting of a chimeric antigen receptor (CAR) and a T cell receptor (TCR).

12. The composition of claim 1, wherein the one or more polynucleic acids comprise one or more homology arms.

13. The composition of claim 1, wherein the target DNA sequence is a genomic DNA sequence.

14. The composition of claim 1, wherein the target DNA sequence is at a genomic safe harbor locus.

15. The composition of claim 1, wherein the target DNA sequence is not a ribosomal DNA sequence.

16. The composition of claim 1, wherein the Cas nickase is a Cas9 nickase.

17. A pharmaceutical composition comprising (a) the composition of claim 1 and (b) a pharmaceutically acceptable excipient.

18. A method of treating a disease or condition in a subject in need thereof, the method comprising administering a therapeutically effective amount of the pharmaceutical composition of claim 17 to the subject.

19. The method of claim 18, wherein the disease or condition is cancer.

20. The method of claim 18, wherein the subject is a human.


SEQUENCE LISTING (113 sequences)

SEQ ID NO: 1 - protein, 116 aa, synthetic construct
  REGION 1..116: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTV

SEQ ID NO: 2 - protein, 107 aa, synthetic construct
  REGION 1..107: note = Description of Artificial Sequence: Synthetic polypeptide
DIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 3 - protein, 46 aa, synthetic construct
  REGION 1..46: note = Description of Artificial Sequence: Synthetic polypeptide
LYCRRLKIQVRKAAITSYEKSDGVYTGLSTRNQETYETLKHEKPPQ

SEQ ID NO: 4 - protein, 35 aa, synthetic construct
  REGION 1..35: note = Description of Artificial Sequence: Synthetic polypeptide
YEDMRGILYAAPQLRSIRGQPGPNHEEDADSYENM

SEQ ID NO: 5 - protein, 62 aa, synthetic construct
  REGION 1..62: note = Description of Artificial Sequence: Synthetic polypeptide
KKVAKKPTNKAPHPKQEPQEINFPDDLPGSNTAAPVQETLHGCQPVTQEDGKESRISVQERQ

SEQ ID NO: 6 - protein, 21 aa, synthetic construct
  REGION 1..21: note = Description of Artificial Sequence: Synthetic peptide
IYIWAPLAGTCGVLLLSLVIT

SEQ ID NO: 7 - protein, 62 aa, synthetic construct
  REGION 1..62: note = Description of Artificial Sequence: Synthetic polypeptide
ALSNSIMYFSHFVPVFLPAKPTTTPAPRPPTPAPTIASQPLSLRPEACRPAAGGAVHTRGLD

SEQ ID NO: 8 - protein, 130 aa, synthetic construct
  REGION 1..130: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVE

SEQ ID NO: 9 - protein, 108 aa, synthetic construct
  REGION 1..108: note = Description of Artificial Sequence: Synthetic polypeptide
LVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 10 - protein, 17 aa, synthetic construct
  REGION 1..17: note = Description of Artificial Sequence: Synthetic peptide
SSGGGGSGGGGSGGGGS

SEQ ID NO: 11 - protein, 7 aa, synthetic construct
  REGION 1..7: note = Description of Artificial Sequence: Synthetic peptide
SGGGGSG

SEQ ID NO: 12 - protein, 4 aa, synthetic construct
  REGION 1..4: note = Description of Artificial Sequence: Synthetic peptide
SGGG

SEQ ID NO: 13 - protein, 4 aa, synthetic construct
  REGION 1..4: note = Description of Artificial Sequence: Synthetic peptide
GSGS

SEQ ID NO: 14 - protein, 432 aa, synthetic construct
  REGION 1..432: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 15 - protein, 436 aa, synthetic construct
  REGION 1..436: note = Description of Artificial Sequence: Synthetic polypeptide
MWLQSLLLLGTVACSISDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 16 - protein, 454 aa, synthetic construct
  REGION 1..454: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQKKVAKKPTNKAPHPKQEPQEINFPDDLPGSNTAAPVQETLHGCQPVTQEDGKESRISVQERQ

SEQ ID NO: 17 - protein, 17 aa, synthetic construct
  REGION 1..17: note = Description of Artificial Sequence: Synthetic peptide
MWLQSLLLLGTVACSIS

SEQ ID NO: 18 - protein, 27 aa, synthetic construct
  REGION 1..27: note = Description of Artificial Sequence: Synthetic peptide
FWVLVVVGGVLACYSLLVTVAFIIFWV

SEQ ID NO: 19 - protein, 25 aa, synthetic construct
  REGION 1..25: note = Description of Artificial Sequence: Synthetic peptide
ILLPLIIGLILLGLLALVLIAFCII

SEQ ID NO: 20 - protein, 45 aa, synthetic construct
  REGION 1..45: note = Description of Artificial Sequence: Synthetic polypeptide
LYCRLKIQVRKAAITSYEKSDGVYTGLSTRNQETYETLKHEKPPQ

SEQ ID NO: 21 - protein, 219 aa, synthetic construct
  REGION 1..219: note = Description of Artificial Sequence: Synthetic polypeptide
QRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 22 - protein, 171 aa, synthetic construct
  REGION 1..171: note = Description of Artificial Sequence: Synthetic polypeptide
PLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 23 - protein, 211 aa, synthetic construct
  REGION 1..211: note = Description of Artificial Sequence: Synthetic polypeptide
MSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIEN

SEQ ID NO: 24 - protein, 607 aa, synthetic construct
  REGION 1..607: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSMSNGYSTDENFRYLISCFRARVKMYIQVEPVLDYLTFLPAEVKEQIQRTVATSGNMQAVELLLSTLEKGVWHLGWTREFVEALRRTGSPLAARYMNPELTDLPSPSFENAHDEYLQLLNLLQPTLVDKLLVRDVLDKCMEEELLTIEDRNRIAAAENNGNESGVRELLKRIVQKENWFSAFLNVLRQTGNNELVQELTGSDCSESNAEIEN

SEQ ID NO: 25 - protein, 615 aa, synthetic construct
  REGION 1..615: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 26 - protein, 567 aa, synthetic construct
  REGION 1..567: note = Description of Artificial Sequence: Synthetic polypeptide
MWLQSLLLLGTVACSISEIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQGSGSPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 27 - protein, 42 aa, synthetic construct
  REGION 1..42: note = Description of Artificial Sequence: Synthetic polypeptide
RLKIQVRKAAITSYEKSDGVYTGLSTRNQETYETLKHEKPPQ

SEQ ID NO: 28 - protein, 42 aa, synthetic construct; identical to SEQ ID NO: 27
  REGION 1..42: note = Description of Artificial Sequence: Synthetic polypeptide

SEQ ID NO: 29 - protein, 24 aa, synthetic construct
  REGION 1..24: note = Description of Artificial Sequence: Synthetic peptide
IYIWAPLAGTCGVLLLSLVITLYC

SEQ ID NO: 30 - protein, 86 aa, synthetic construct
  REGION 1..86: note = Description of Artificial Sequence: Synthetic polypeptide
ALSNSIMYFSHFVPVFLPAKPTTTPAPRPPTPAPTIASQPLSLRPEACRPAAGGAVHTRGLDIYIWAPLAGTCGVLLLSLVITLYC

SEQ ID NO: 31 - protein, 83 aa, synthetic construct
  REGION 1..83: note = Description of Artificial Sequence: Synthetic polypeptide
ALSNSIMYFSHFVPVFLPAKPTTTPAPRPPTPAPTIASQPLSLRPEACRPAAGGAVHTRGLDIYIWAPLAGTCGVLLLSLVIT

SEQ ID NO: 32 - protein, 255 aa, synthetic construct
  REGION 1..255: note = Description of Artificial Sequence: Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESSGGGGSGGGGSGGGGSLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTV

SEQ ID NO: 33 - protein, 240 aa, synthetic construct
  REGION 1..240: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIK

SEQ ID NO: 34 - protein, 26 aa, synthetic construct
  REGION 1..26: note = Description of Artificial Sequence: Synthetic peptide
IYLIIGICGGGSLLMVFVALLVFYIT

SEQ ID NO: 35 - DNA, 1075 bp, synthetic construct
  misc_feature 1..1075: note = Description of Artificial Sequence: Synthetic polynucleotide
  source 1..1075: mol_type = other DNA
taatacgactcactatagggagaaagacgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatggattataaggatgacgatgataaatga

SEQ ID NO: 36 - DNA, 5751 bp, synthetic construct
  misc_feature 1..5751: note = Description of Artificial Sequence: Synthetic polynucleotide
  source 1..5751: mol_type = other DNA
taatacgactcactatagggagaaagacgccaccatgacaggttcaaatagtcacattacgattctcactctgaatataaatgggctgaattctgcaattaaacggcacaggcttgcttcctggataaagtctcaagacccctcagtgtgctgtattcaggaaacgcatctcacgtgcagggacacccatcggctgaaaataaaaggctggcggaagatctaccaagccaatggaaaacaaaagaaggctggggtggcgatacttgtaagcgataaaacagactttaaaccaactaagatcaaacgggacaaagagggccattacatcatggtaaagggtagtattcaacaagaggagctgactatcctgaatatttatgcacctaatactggagcccccagattcataaagcaagtgttgagtgaccttcaacgcgacctcgactcccacactctgatcatgggagactttaacaccccgctgtccactctcgacagatctactagacagaaagtcaacaaggatacacaggaactgaacagtgctctccaccaagcggaccttatcgacatctacagaacactccaccccaaaagcacagaatataccttcttttcagcccctcaccacacctattccaaaattgaccacattgtggggagtaaagcccttctctccaaatgtaaacggaccgaaattatcactaactatctctccgaccacagtgcaataaaacttgaattgcgaattaagaatctcactcaaagtagatccacgacatggaaactgaacaatctcctcttgaatgactactgggtgcataacgaaatgaaggctgaaataaagatgttctttgagaccaacgaaaacaaagacaccacgtaccagaatctctgggacgctttcaaagcagtgtgtcgaggaaaatttattgcactgaatgcttacaagcggaagcaggaaagatccaaaatagacaccctgactagccaacttaaagaactggaaaagcaagagcaaactcatagcaaagctagccgtcgccaagaaattacgaaaatcagagctgaactgaaggaaattgagacacagaaaaccctgcaaaagataaatgaaagccgcagctggttctttgaacgcatcaacaaaatcgataggccacttgctcgccttatcaagaagaaaagggagaagaatcaaatcgacactataaagaatgataaaggcgatataaccaccgatcccacagaaattcaaacaaccatacgcgaatactacaaacacctctacgccaataaactcgaaaatctcgaggaaatggatacattcctcgacacgtacacccttcccaggctgaaccaggaagaagttgaatcactgaatcggcctatcacggggagtgaaatagtagctatcatcaattcactccctaccaagaagtcacccggacctgatggattcaccgccgaattctaccagagatacatggaagaactggtgcccttcttgctgaaacttttccaaagtattgagaaagagggaatacttccaaactcattttatgaggcatccatcattctgatcccgaagcccggcagggacacgaccaagaaagagaattttcgaccaatctcattgatgaacattgatgcaaagatcctcaataaaatactggcaaatcggattcagcagcacataaagaagctgatccaccatgatcaagtaggcttcatccccggtatgcaaggttggttcaatatacgaaaatcaatcaatgttatccagcatataaaccgggccaaagacaagaaccacatgattattagtatcgatgctgagaaagcctttgacaaaatacaacaacccttcatgctgaaaacattgaataagctgggaattgatggcacctacttcaaaatcatcagagccatatatgacaaaccaacagcaaatatcattctgaatggtcagaaattggaagcattccccttgaaaaccggcacacggcagggttgccctctgtcaccactcctcttcaacatcgtgttggaagttcttgcccgcgcaatccggcaggaaaaggaaatcaagggcattcaactgggcaaagaggaagttaaattgagcctgtttgcagacgacatgatcgtctatttggaaaaccccatagttagtgcacaaaatctgctgaagttgatcagtaatttctccaaagtgagtgggtacaaaatcaatgtgcaaaagagccaagctttcttgtacaccaacaacaggcaaactgagtctcaaatcatgggcgaactccccttcgtgattgcatccaagcggatcaaatacctggggattcaattgactcgtgatgtgaaggacctcttcaaggagaactacaaacccctgctcaaggaaatcaaagaggacacaaacaaatggaagaacattccatgctcttgggtgggaaggatcaatatcgtcaaaatggccatcctgcccaaggtaatttacaggttcaatgctatacccatcaagctccccatgacattcttcacagaacttgaaaagacgacgctgaagttcatttggaaccagaaacgtgccaggattgctaaatctattctctcccaaaagaacaaagctggcggaatcacactcccagacttcaaactttactacaaggcgaccgtgacgaaaacggcttggtactggtaccaaaacagggatatagatcaatggaaccgaacggagcccagcgaaattatgcctcatatatacaactatctgatctttgacaaaccggagaagaacaagcaatggggaaaggatagtctgtttaataaatggtgctgggaaaactggctcgcaatctgtaggaagctgaaactggatccattcttgacgccttatacaaagataaattcccgatggattaaagatctcaacgtgaaacccaaaacaattaaaaccctcgaggaaaacctgggtattacgattcaggacattggggtgggaaaggacttcatgtccaaaaccccaaaagcgatggcaaccaaagacaaaatcgacaaatgggatctcataaaacttaagtcattttgcacagctaaagaaacgacaattagggtgaaccgacaaccgaccacttgggagaaaatcttcgcaacatacagttctgacaaaggcctgatttccaggatctacaatgaattgaaacaaatttacaagaagaagacgaacaaccctataaagaaatgggccaaggacatgaacagacacttctctaaggaagacatttatgcagccaagaaacacatgaagaaatgcagctcttcactggcaatcagggaaatgcaaatcaaaacaacaatgagatatcatctcacacccgtcagaatggccatcattaagaagagcggaaacaaccggtgctggcgtggttgcggagaaatcggtactctccttcactgttggtgggactgtaaactcgttcaaccactgtggaagtctgtgtggcggttcctcagagatctggaactcgaaatcccatttgacccagccatccctctcctgggtatatacccgaatgagtataaatcctgctgctataaagacacctgcacaaggatgtttattgcagctctcttcacaatcgcgaagacgtggaaccaacccaaatgtccgactatgattgactggattaagaagatgtggcacatatacactatggaatactatgctgcgatcaagaacgatgagttcatatcatttgtgggcacatggatgaaactcgaaaccatcatactctctaaattgagtcaagaacagaaaactaaacaccgtatattttccctgatcggtgggaattagctacaaagacgatgacgacaaggaccatggagacggtgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgacgtctcagctgacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacatatgtaactaacctgcacaatgtgcacatgtaccctaaaacttagagtataatggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 37 - DNA, 18285 bp, synthetic construct
  misc_feature 1..18285: note = Description of Artificial Sequence: Synthetic polynucleotide
  source 1..18285: mol_type = other DNA
cggccgcggggggaggagccaagatggccgaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttccgagtcaaagaaaggggtgacggacgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagatcaaactgcaaggcggcaacgaggctgggggaggggcgcccgccattgcccaggcttgcttaggtaaacaaagcagcagggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgatgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaaggaaagcccatcagactaacagcggatctctcggcagaaaccctacaagccagaagagagtgggggccaatattcaacattcttaaagaaaagaattttcaacccagaatttcatatccagccaaactaagcttcataagtgaaggagaaataaaatactttatagacaagcaaatgttgagagattttgtcaccaccaggcctgccctaaaagagctcctgaaggaagcgctaaacatggaaaggaacaaccggtaccagccgctgcaaaatcatgccaaaatgtaaagaccatcaagactaggaagaaactgcatcaactaatgagcaaaatcaccagctaacatcataatgacaggatcaacttcacacataacaatattaactttaaatataaatggactaaattctgcaattaaaagacacagactggcaagttggataaagagtcaagacccatcagtgtgctgtattcaggaaacccatctcacgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctcagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatcgaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaaccggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcacaattgcttcaaagagaataaaatacctaggaatccaacttacaagggatgtgaaggacctcttcaaggagaactacaaaccactgctcaaggaaataaaagaggagacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaatttttgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgatcgactctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcactcgccgatagtggaaaccgacgccccagcactcgtccgagggcaaaggaataggggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgcattgctgcaggcgcagaactggtaggtatggaagatctctagaagctgggtaccagctgctagcaagcttgctagcggccggctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgccg

SEQ ID NO: 38 - DNA, 7264 bp, synthetic construct
  misc_feature 1..7264: note = Description of Artificial Sequence: Synthetic polynucleotide
  source 1..7264: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 39 - DNA, 18852 bp, synthetic construct
  misc_feature 1..18852: note = Description of Artificial Sequence: Synthetic polynucleotide
  source 1..18852: mol_type = other DNA
cggccgcggggggaggagccaagatggccgaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttccgagtcaaagaaaggggtgacggacgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagatcaaactgcaaggcggcaacgaggctgggggaggggcgcccgccattgcccaggcttgcttaggtaaacaaagcagcagggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgatgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaaggaaagcccatcagactaacagcggatctctcggcagaaaccctacaagccagaagagagtgggggccaatattcaacattcttaaagaaaagaattttcaacccagaatttcatatccagccaaactaagcttcataagtgaaggagaaataaaatactttatagacaagcaaatgttgagagattttgtcaccaccaggcctgccctaaaagagctcctgaaggaagcgctaaacatggaaaggaacaaccggtaccagccgctgcaaaatcatgccaaaatgtaaagaccatcaagactaggaagaaactgcatcaactaatgagcaaaatcaccagctaacatcataatgacaggatcaacttcacacataacaatattaactttaaatataaatggactaaattctgcaattaaaagacacagactggcaagttggataaagagtcaagacccatcagtgtgctgtattcaggaaacccatctcacgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctcagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatcgaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaaccggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcacaattgcttcaaagagaataaaatacctaggaatccaacttacaagggatgtgaaggacctcttcaaggagaactacaaaccactgctcaaggaaataaaagaggagacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaatttttgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctcatgttctcgtaggagtcggcgtcctcttcgtggttaggtccaggttggcctctgatagaccgcagctgaggagcggcgtacagaatgcctctcatgtcctcatagctgccgctgccttgtggaggcttctcgtgcttcagtgtctcgtatgtctcttgattccgggtgctcaggccggtgtacacgccatcagatttctcgtagctggtgatggcggccttccgcacttggatcttcagccgtctgcagtacagggtgatgaccagagacagcagcaggacaccacatgtgccagccagaggggcccaaatgtagatatccaggcctctggtatgcacagctccgcctgcagcaggtctacaggcttcaggtctgagagacagaggctggctggcgattgtaggagctggtgtaggtggtctaggagcgggtgttgttgtaggcttggcgggcagaaacacgggcacgaagtggctgaagtacatgatgctattgctcagggctccgcttcctccgccgcctgatttgatttccagcttggtgcctccgccaaatgtccaagggctctcgtcgtactgctggcagtagtagatgccgaagtcctcgtactgcaggctgctgattgtcagggtgtagtcggtgccagagccgctgccagaaaatctgcttggcacgccgctttccagtctgttggcccggtagatcagtgtcttaggggccttgccaggcttctgctggaaccagctcaggtagctgttgatgtcctggctggctctacaggtgatggtcactctatcgcccacagaggcagacaggctgctagggctctgtgtcatctggatatcagagccaccaccgccagatccaccgccacctgatcctccgcctccgctagaaactgtcactgtggtgccctggccccacacatcgaagtaccagtcgtagcctcttctggtgcagaagtacacggcggtatcctcggctctcaggctgttgatctgcaggtaggcggtgttcttgctgtcgtccaggctgaaggtgaatctgcccttaaagctatcggcgtaggttggctcgccggtgtgggtattgatccagcccatccactcaaggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttttccaggggcctgtcggacccagttcatgccgtagttggtgaaggtgtagccgctggcggcacagctgattctgacagatccgccaggtttcacaagtccgccgccagactgaaccagctggatctcagagatgctacaggccactgttcccagcagcagcagagactgcagccacatctggtggcgaattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgatcgactctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcactcgccgatagtggaaaccgacgccccagcactcgtccgagggcaaaggaataggggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgcattgctgcaggcgcagaactggtaggtatggaagatctctagaagctgggtaccagctgctagcaagcttgctagcggccggctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgccg

SEQ ID NO: 40 - DNA, 19625 bp, synthetic construct
  misc_feature 1..19625: note = Description of Artificial Sequence: Synthetic polynucleotide
  source 1..19625: mol_type = other DNA
cggccgcggggggaggagccaagatggccgaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttccgagtcaaagaaaggggtgacggacgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagatcaaactgcaaggcggcaacgaggctgggggaggggcgcccgccattgcccaggcttgcttaggtaaacaaagcagcagggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgatgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaaggaaagcccatcagactaacagcggatctctcggcagaaaccctacaagccagaagagagtgggggccaatattcaacattcttaaagaaaagaattttcaacccagaatttcatatccagccaaactaagcttcataagtgaaggagaaataaaatactttatagacaagcaaatgttgagagattttgtcaccaccaggcctgccctaaaagagctcctgaaggaagcgctaaacatggaaaggaacaaccggtaccagccgctgcaaaatcatgccaaaatgtaaagaccatcaagactaggaagaaactgcatcaactaatgagcaaaatcaccagctaacatcataatgacaggatcaacttcacacataacaatattaactttaaatataaatggactaaattctgcaattaaaagacacagactggcaagttggataaagagtcaagacccatcagtgtgctgtattcaggaaacccatctcacgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctcagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatcgaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaaccggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcacaattgcttcaaagagaataaaatacctaggaatccaacttacaagggatgtgaaggacctcttcaaggagaactacaaaccactgctcaaggaaataaaagaggagacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaatttttgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatagggccgggattctcctccacgtcaccgcatgttagaagacttcctctgccctccatgttctcgtaggagtcggcgtcctcttcgtggttaggtccaggttggcctctgatagaccgcagctgaggagcggcgtacagaatgcctctcatgtcctcatagctgccgctgccttgtggaggcttctcgtgcttcagtgtctcgtatgtctcttgattccgggtgctcaggccggtgtacacgccatcagatttctcgtagctggtgatggcggccttccgcacttggatcttcagccgtctgcagtacagggtgatgaccagagacagcagcaggacaccacatgtgccagccagaggggcccaaatgtagatatccaggcctctggtatgcacagctccgcctgcagcaggtctacaggcttcaggtctgagagacagaggctggctggcgattgtaggagctggtgtaggtggtctaggagcgggtgttgttgtaggcttggcgggcagaaacacgggcacgaagtggctgaagtacatgatgctattgctcagggctccgcttcctccgccgcctgatttgatttccagcttggtgcctccgccaaatgtccaagggctctcgtcgtactgctggcagtagtagatgccgaagtcctcgtactgcaggctgctgattgtcagggtgtagtcggtgccagagccgctgccagaaaatctgcttggcacgccgctttccagtctgttggcccggtagatcagtgtcttaggggccttgccaggcttctgctggaaccagctcaggtagctgttgatgtcctggctggctctacaggtgatggtcactctatcgcccacagaggcagacaggctgctagggctctgtgtcatctggatatcagagccaccaccgccagatccaccgccacctgatcctccgcctccgctagaaactgtcactgtggtgccctggccccacacatcgaagtaccagtcgtagcctcttctggtgcagaagtacacggcggtatcctcggctctcaggctgttgatctgcaggtaggcggtgttcttgctgtcgtccaggctgaaggtgaatctgcccttaaagctatcggcgtaggttggctcgccggtgtgggtattgatccagcccatccactcaaggccttttccaggggcctgtcggacccagttcatgccgtagttggtgaaggtgtagccgctggcggcacagctgattctgacagatccgccaggtttcacaagtccgccgccagactgaaccagctggatctcagagatgctacaggccactgttcccagcagcagcagagactgcagccacattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgatcgactctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcactcgccgatagtggaaaccgacgccccagcactcgtccgagggcaaaggaataggggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgcattgctgcaggcgcagaactggtaggtatggaagatctctagaagctgggtaccagctgctagcaagcttgctagcggccggctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgccg

SEQ ID NO: 41 - DNA, 19730 bp, synthetic construct
  misc_feature 1..19730: note = Description of Artificial Sequence: Synthetic polynucleotide
  source 1..19730: mol_type = other DNA
cggccgcggggggaggagccaagatggccgaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttccgagtcaaagaaaggggtgacggacgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagatcaaactgcaaggcggcaacgaggctgggggaggggcgcccgccattgcccaggcttgcttaggtaaacaaagcagcagggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagctggagatctgagaacgggcagactgcctcctcaagtgggtccctgacccctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgatgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaaggaaagcccatcagactaacagcggatctctcggcagaaaccctacaagccagaagagagtgggggccaatattcaacattcttaaagaaaagaattttcaacccagaatttcatatccagccaaactaagcttcataagtgaaggagaaataaaatactttatagacaagcaaatgttgagagattttgtcaccaccaggcctgccctaaaagagctcctgaaggaagcgctaaacatggaaaggaacaaccggtaccagccgctgcaaaatcatgccaaaatgtaaagaccatcaagactaggaagaaactgcatcaactaatgagcaaaatcaccagctaacatcataatgacaggatcaacttcacacataacaatattaactttaaatataaatggactaaattctgcaattaaaagacacagactggcaagttggataaagagtcaagacccatcagtgtgctgtattcaggaaacccatctcacgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctcagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacatttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatcgaccaataacaggctctgaaattgtggcaataatcaatagtttaccaaccaaaaagagtccaggaccagatggattcacagccgaattctaccagaggtacaaggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccagcagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaaccggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtttatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcacaattgcttcaaagagaataaaatacctaggaatccaacttacaagggatgtgaaggacctcttcaaggagaactacaaaccactgctcaaggaaataaaagaggagacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaatttttgcaacctactcatctgacaaagggctaatatccagaatctacaatgaactcaaacaaatttacaagaaaaaaacaaacaaccccatcaaaaagtgggcgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacggatccgaattctcgacggatcgatccgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatagggccgggattctcctccacgtcaccgcatgttagaagacttcctctgccctctcttggaggcagggcctgcatgtgcagggcatcgtaggtatccttggtggctgtgctcagtccctggtacagtccatcgtggcccttgcctcttcttctctcgcccttcatgccgatctcgctgtaggcctcggccatcttgtctttctgcagctcattatacaggccctcttgaggattctttctccgctggggcttgccgcccatctcaggatctctgcctctccgcttatccagcacgtcgtactcttctcttctccccaggttcagctcgttgtacagctgattctggccctgctggtaagcaggagcgtcggcggatctgctgaacttcactctgcagtacagggtgatgaccagagagagcagcagaacgccacatgtgccagccagaggggcccaaatgtagatatccaggcctctggtatgcacagctccgccagctgcaggtctacaggcttcaggtctgagagacagaggctggctggcgattgtaggagctggtgtaggtggtctaggagcgggtgttgttgtaggcttggcgggcagaaacacgggcacgaagtggctgaagtacatgatgctattgctcagggctccgcttcctccgcctccgctagaagaaactgtgaccagggtgccctgtccccaaacatccatggcgtagaagccgtcgcctccccatctagaacagtagtacacggcggtgtcctcggctctcaggctgttcatctgcaggtaggcggtgttcttgctggtgtcggcgctgatggtgaatctgcccttcacgctatcggcgtatctggtgtagccgttggtggggtagattctggcgacccattcaagtccctttccaggggcctgtcggacccagtggatgtaggtgtccttgatgttgaagccgctggcggcacaagacagtctcagagagccgccaggctgaacaagtcctccgccagattcaaccagctgcacctcagatccttcgccagatccaggctttccagagccgctggtgctgcctgttctcttgatttccaccttggtgccctggccaaaggttggaggtgtggtgtagtgctgctggcagtagtaggtggcgaagtcctcaggctgcaggctagagatggtcagggtgaagtcggtgccagatctgctgccgctgaatctgcttggcacgccgctgtacagaaagctggcgctgtagatcagcagcttaggggcttttccaggcttctgctgataccaggccacggcggtattcacatcctggctggctctacaggtgatggtcactctatcgcccacagaggcagacaggctgctagggctctgtgtcatctggatgtcgctgatgctgcaggccactgttcccagcagcagcagagactgcagccacattcgaagcttgagctcgagatctgagtccggtagcgctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattagcccgggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgatcgactctagaggatcgatccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgcatgcctgcaggtcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcactcgccgatagtggaaaccgacgccccagcactcgtccgagggcaaaggaataggggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtctggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgcattgctgcaggcgcagaactggtaggtatggaagatctctagaagctgggtaccagctgctagcaagcttgctagcggccggctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgccg

SEQ ID NO: 42 - DNA, 7291 bp, synthetic construct
  misc_feature 1..7291: note = Description of Artificial Sequence: Synthetic polynucleotide
  source 1..7291: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagcccgccgccaagagggtgaagctggactaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 43 - DNA, 7376 bp, synthetic construct
  misc_feature 1..7376: note = Description of Artificial Sequence: Synthetic polynucleotide
  source 1..7376: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcggccgggcgcggtggctcacgcctgtaatcccagcactttgggaggccgaggcgggaggatcgcagttcgagaccagcgcgagaccccgtctctacaaaaatacaaaaattagcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 44 - DNA, 14122 bp, synthetic construct
  misc_feature 1..14122: note = Description of Artificial Sequence: Synthetic polynucleotide
  source 1..14122: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagcacgagatctgagtccggtaggcctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgc

SEQ ID NO: 45 - DNA, 14124 bp, synthetic construct
  misc_feature 1..14124: note = Description of Artificial Sequence: Synthetic polynucleotide
  source 1..14124: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagttaaaacagctgtgggttgtcacccacccacagggtccactgggcgctagtacactggtatctcggtacctttgtacgcctgttttataccccctccctgatttgcaacttagaagcaacgcaaaccagatcaatagtaggtgtgacataccagtcgcatcttgatcaagcacttctgtatccccggaccgagtatcaatagactgtgcacacggttgaaggagaaaacgtccgttacccggctaactacttcgagaagcctagtaacgccattgaagttgcagagtgtttcgctcagcactccccccgtgtagatcaggtcgatgagtcaccgcattccccacgggcgaccgtggcggtggctgcgttggcggcctgcctatggggtaacccataggacgctctaatacggacatggcgtgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttctttttattcttgtattggctgcttatggtgacaattaaagaattgttaccatatagctattggattggccatccagtgtcaaacagagctattgtatatctctttgttggattcacacctctcactcttgaaacgttacacaccctcaattacattatactgctgaacacgaagcgtacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagcacgagatctgagtccggtaggcctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgc

SEQ ID NO: 46 - DNA, 13439 bp, synthetic construct
  misc_feature 1..13439: note = Description of Artificial Sequence: Synthetic polynucleotide
  source 1..13439: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgggaagcggacagtgtactaattatgctctcttgaaattggctggagatgttgagagcaaccctggacctatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagcacgagatctgagtccggtaggcctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgc

SEQ ID NO: 47 - DNA, 13436 bp, synthetic construct
  misc_feature 1..13436: note = Description of Artificial Sequence: Synthetic polynucleotide
  source 1..13436: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgggaagcggagctactaacttcagcctgctgaagcaggctggagacgtggaggagaaccctggacctatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagcacgagatctgagtccggtaggcctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgc

SEQ ID NO: 48 - DNA, 13433 bp, synthetic construct
  misc_feature 1..13433: note = Description of Artificial Sequence: Synthetic polynucleotide
  source 1..13433: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgggaagcggagagggcagaggaagtctgctaacatgcggtgacgtcgaggagaatcctggacctatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcgaacaaacgacccaacacccgtgcgttttattctgtctttttattgccgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggccaggtgagtccaggagatgtttcagcactgttgcctttagtctcgaggcaacttagacaactgagtattgatctgagcacagcagggtgtgagctgtttgaagatactggggttgggggtgaagaaactgcagaggactaactgggctgagacccagtggcaatgttttagggcctaaggaatgcctctgaaaatctagatggacaactttgactttgagaaaagagaggtggaaatgaggaaaatgacttttctttattagatttcggtagaaagaactttcatctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagctgggtgtgtagttatctggaggccaggctggagctctcagctcactatgggttcatctttattgtctcctttcatctcaacagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaattcgaagcttgagcacgagatctgagtccggtaggcctagcggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgc

SEQ ID NO: 49 - DNA, 9241 bp, synthetic construct
  misc_feature 1..9241: note = Description of Artificial Sequence: Synthetic polynucleotide
  source 1..9241: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacgccatggccagcaacttcacccagttcgtgctggtggacaacggcggcaccggcgacgtgaccgtggcccccagcaacttcgccaacggcatcgccgagtggatcagcagcaacagcagaagccaggcctacaaggtgacctgcagcgtgagacagagcagcgcccagaacagaaagtacaccatcaaggtggaggtgcccaagggcgcctggagaagctacctgaacatggagctgaccatccccatcttcgccaccaacagcgactgcgagctgatcgtgaaggccatgcagggcctgctgaaggacggcaaccccatccccagcgccatcgccgccaacagcggcatctacgactacaaagacgatgacgacaagtaaagcaacctacaaacgggtggaggatcaccccacccgacacttcacaatcaaggggtacaatacacaagggtggaggaacaccccaccctccagacacattacacagaaatccaatcaaacagaagcaccatcagggcttctgctaccaaatttatctcaaaaaactacaacaaggaatcaccatcagggattccctgtgcaatatacgtcaaacgagggccacgacgggaggacgatcacgcctcccgaatatcggcatgtctggctttcgaattcagtgcgtggagcatcagcccacgcagccaatcagagtcgaatacaagtcgactttcgcgaagagcatcagccttcgcgccattcttacacaaaccacactctcccctacaggaacagcatcagcgttcctgcccagtacccaactcaagaaaatttatgtccccatgcagcatcagcgcatgggccccaagaatacatccccaacaaaatcacatccgagcaccaacagggctcggagtgttgtttcttgtccaactggacaaaccctccatggaccatcaggccatggactctcaccaacaagacaaaaactactcttctcgaagcagcatcagcgcttcgaaacactcgagcatacattgtgcctatttcttgggtggacgatcacgccacccatgctctcacgaatttcaaaacacggacaaggacgagcaccaccagggctcgtcgttccacgtccaatacgattacttacctttcgggatcacgatcacggatcccgcagctacatcacttccactcaggacattcaagcatgcacgatcacggcatgctccacaagtctcaaccacagaaactaccaaatgggttcagcaccagcgaacccactcctacctcaaacctcttcccacaaaactggcaagcaggatcaccgcttgcccattccaacataccaaatcaaaaacaattactggtacagcatcagcgtaccagcccacatctctcactactatcaaaaaccaaaccgttcagcaacagcgaacggtacacacggaaaaatcaactggtttacaaatacgaaagacgatcacgctttcgtccagcgcaaactattacgaaaaacatccgacgggaagagcaacagccttcccgcggcggaaaacctcacaaaaacacgacaaacggatgcacgaacacggcatccgccgacaacccacaaacttacaaccaggcaaacggtgcaggatcaccgcaccgtacatcaaacacctcagatctcatgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 50 - DNA, 7309 bp, synthetic construct
  misc_feature 1..7309: note = Description of Artificial Sequence: Synthetic polynucleotide
  source 1..7309: mol_type = other DNA
taatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatggtcataggaacttacatttcgattattaccttaaacgtgaatgggttaaatgccccaaccaagagacatcggctggctgaatggattcagaaacaggacccctatatttgctgtctgcaggagacccacttccgtcctcgcgacacatacagactgaaagtgaggggctggaaaaagatcttccatgccaatggaaatcaaaagaaagctggagtggctattctcatctcagataaaattgacttcaaaataaagaatgttactcgagataaggagggacactacataatgatccaggggtccatccaagaagaggatataactattattaatatttatgcacccaacattggcgcccctcagtacatcaggcagctgcttacagctatcaaggaggaaatcgacagtaacacgattatcgtgggggactttaacaccagccttactccgatggatagatcatccaaaatgaaaataaataaggaaacagaggctcttaatgacaccattgaccagatagatctgattgatatatataggacattccatccaaaaactgccgattacactttcttcagcagtgcgcatggaaccttctccaggatagatcacatcttgggtcacaaaagtagcctcagtaagtttaagaaaattgaaatcattagcagcatcttttctgaccataacgctatgcgcctggagatgaatcacagggagaagaacgtaaagaagacaaacacctggaggctgaacaatacgctgctaaataaccaagagatcactgaggaaatcaaacaggaaataaaaaaatacttggagacaaatgacaatgaaaacacgaccacccagaacttgtgggatgcagctaaagcggttctgagagggaagtttatagctattcaagcctaccttaagaaacaggaaaaatctcaagtgaacaatttgaccttacacctaaagaaactggagaaggaggagcagaccaaacccaaagtgagcaggaggaaagaaatcatcaagatcagagccgaaatcaatgaaatagaaactaagaagacaattgccaagatcaataaaactaaatcctggttctttgagaagatcaacaaaattgataagccattagccagactcatcaagaaaaagagggagaggactcagatcaataagatcagaaatgagaaaggggaagttacaaccgacaccgcggagattcagaacatcctgagagactactacaagcaactttatgccaataaaatggacaacctggaagaaatggacaaattcctggaaaggtataaccttccccggctgaaccaggaggagactgaaaatatcaaccgcccaatcacaagtaatgagattgagactgtgattaagaatcttccaactaacaaaagtcccggccccgatggcttcacaggtgaattctatcagacctttcgggaggagttgacacccatccttctcaagctcttccaaaaaattgcagaggagggcacactcccgaactcattctatgaggccaccatcaccctgatcccaaagcccgacaaggacactacaaagaaagaaaattaccgaccaatttccctgatgaatatcgatgccaagatcctcaacaaaatcttggcaaacagaatccagcagcacattaagaggatcatacaccacgatcaggtgggctttatcccggggatgcaaggattcttcaatatccgcaaatcaatcaatgtgatccaccatattaacaagttgaagaagaagaaccatatgatcatctccatcgatgcagagaaagcttttgacaaaattcaacacccatttatgatcaaaactctccagaaggtgggcatcgaggggacctacctcaacataattaaggccatctatgataagcccacagccaacatcattctcaatggtgaaaagctgaaggcatttcctctgcggtccggaacgagacagggatgtcctctctctcctcttctgttcaacatcgttctggaagtcctagccaccgctatccgcgaggaaaaggaaattaaaggcatacagattggaaaggaagaggtaaaactgtctctgtttgcggatgatatgatactgtacatagagaatcctaaaactgccacccggaagctgttggagctaattaatgagtatggtaaggtcgccggttacaagattaatgctcagaagtctcttgctttcctgtacactaatgatgaaaagtctgaacgggaaattatggagacactcccctttaccattgcaaccaaacgtattaaataccttggcattaacctgcctaaggagacaaaagacctgtatgctgaaaactataagacactgatgaaagagattaaagatgataccaaccggtggcgggatatcccatgttcttggattggcagaatcaacattgtgaagatgagcatcctgcccaaggccatctacagattcaatgccatccctatcaaattacctatggcattttttacggagctggaacagatcatcttaaaatttgtgtggcgccacaagcggccccgaatcgccaaagcggtcttgaggcagaagaatggcgctgggggaatccgactccctgacttcagattgtactacaaagctaccgtcatcaagacaatctggtactggcacaagaacagaaacatcgatcagtggaacaagatcgaaagccctgagattaacccccgcacctatggtcaactgatctatgacaaagggggcaaggatatacaatggcgcaaggacagcctcttcaataagtggtgctgggaaaactggacagccacctgcaagcgtatgaagctggagtactccctgacaccatacacaaaaataaactcaaagtggattcgagacctcaatattcggctggacactataaaactcctggaggagaacattgggcgtacactctttgacattaatcatagcaagatctttttcgatccccctcctcgtgtaatggaaataaaaacaaaaataaacaagtgggatctgatgaaacttcagagcttttgcaccgcaaaggagaccataaacaagacgaagcgccaaccctcagaatgggagaaaatatttgcgaatgagtctacggacaaaggcttaatctccaaaatatataagcagctcattcagctcaatatcaaggaaacaaacaccccgatccaaaagtgggcagaggacctaaatcggcatttctccaaggaagacatccagacggccacgaagcacatgaagcgatgctcaacttccctgattattcgcgaaatgcagatcaagactactatgcgctatcacctcactcctgttcggatgggcatcatccggaaatctacaaacaacaagtgctggagagggtgtggcgaaaagggaaccctcttgcattgttggtgggagtgtaagttgatccagccactatggcggaccatatggaggttccttaaaaaactgaagattgagctgccatatgacccagcaatcccactgctgggcatatacccggagaaaaccgtgattcagaaagacacttgcacccgaatgttcattgcagcattgtttacaatagccaggtcatggaagcagcctaagtgcccctcgacagacgagtggatcaagaagatgtggtacatttatactatggaatattacagcgccatcaaacgcaacgaaattgggtcttttctggagacgtggatggatctagagactgtcatccagagtgaggtaagtcagaaagagaagaacaaatatcgtattttaacgcatatttgtggaacctggaagaatggtacagatgagccggtctgccgaaccgagattgagacccagatggactacaaagacgatgacgacaagtgaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 51 - RNA, 61 nt, synthetic construct
  misc_feature 1..61: note = Description of Artificial Sequence: Synthetic oligonucleotide
  source 1..61: mol_type = other RNA
actcctccccatcctctccctctgtccctctgtccctctgaccctgcactgtcccagcacc

SEQ ID NO: 52 - RNA, 118 nt, synthetic construct
  misc_feature 1..118: note = Description of Artificial Sequence: Synthetic polynucleotide
  source 1..118: mol_type = other RNA
caggacacagccttggatcaggacagagacttgggggccatcctgcccctccaacccgacatgtgtacctcagctttttccctcacttgcatcaataaagcttctgtgtttggaacag

SEQ ID NO: 53 - RNA, 76 nt, synthetic construct
  misc_feature 1..76: note = Description of Artificial Sequence: Synthetic oligonucleotide
  source 1..76: mol_type = other RNA
ggaaggtttttcttttcctgaggcgaaagtctcaggttttgctttttggcctttcttaaaaaaaaaaaaagcaaaa

SEQ ID NO: 54 - RNA, 93 nt, synthetic construct
  misc_feature 1..93: note = Description of Artificial Sequence: Synthetic oligonucleotide
  source 1..93: mol_type = other RNA
gaaggtttttcttttcctgagaaaacaacacgtattgttttctcaggttttgctttttggcctttttctagcttaaaaaaaaaaaaagcaaaa

SEQ ID NO: 55 - DNA, 19 bp, unidentified
  source 1..19: mol_type = other DNA
agatgtgtataagagacag

SEQ ID NO: 56 - DNA, 19 bp, unidentified
  misc_feature 1..19: note = Description of Unknown: Transposon end sequence
  source 1..19: mol_type = other DNA
ctgtctcttatacacatct

SEQ ID NO: 57 - protein, 338 aa, synthetic construct [sequence omitted]

SEQ ID NO: 58 - DNA, 1017 bp, synthetic construct
  misc_feature 1..1017: note = Description of Artificial Sequence: Synthetic polynucleotide
  source 1..1017: mol_type = other DNA
atgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtga

SEQ ID NO: 59 - protein, 1275 aa, synthetic construct
  REGION 1..1275: note = Description of Artificial Sequence: Synthetic polypeptide
MTGSNSHITILTLNINGLNSAIKRHRLASWIKSQDPSVCCIQETHLTCRDTHRLKIKGWRKIYQANGKQKKAGVAILVSDKTDFKPTKIKRDKEGHYIMVKGSIQQEELTILNIYAPNTGAPRFIKQVLSDLQRDLDSHTLIMGDFNTPLSTLDRSTRQKVNKDTQELNSALHQADLIDIYRTLHPKSTEYTFFSAPHHTYSKIDHIVGSKALLSKCKRTEIITNYLSDHSAIKLELRIKNLTQSRSTTWKLNNLLLNDYWVHNEMKAEIKMFFETNENKDTTYQNLWDAFKAVCRGKFIALNAYKRKQERSKIDTLTSQLKELEKQEQTHSKASRRQEITKIRAELKEIETQKTLQKINESRSWFFERINKIDRPLARLIKKKREKNQIDTIKNDKGDITTDPTEIQTTIREYYKHLYANKLENLEEMDTFLDTYTLPRLNQEEVESLNRPITGSEIVAIINSLPTKKSPGPDGFTAEFYQRYMEELVPFLLKLFQSIEKEGILPNSFYEASIILIPKPGRDTTKKENFRPISLMNIDAKILNKILANRIQQHIKKLIHHDQVGFIPGMQGWFNIRKSINVIQHINRAKDKNHMIISIDAEKAFDKIQQPFMLKTLNKLGIDGTYFKIIRAIYDKPTANIILNGQKLEAFPLKTGTRQGCPLSPLLFNIVLEVLARAIRQEKEIKGIQLGKEEVKLSLFADDMIVYLENPIVSAQNLLKLISNFSKVSGYKINVQKSQAFLYTNNRQTESQIMGELPFVIASKRIKYLGIQLTRDVKDLFKENYKPLLKEIKEDTNKWKNIPCSWVGRINIVKMAILPKVIYRFNAIPIKLPMTFFTELEKTTLKFIWNQKRARIAKSILSQKNKAGGITLPDFKLYYKATVTKTAWYWYQNRDIDQWNRTEPSEIMPHIYNYLIFDKPEKNKQWGKDSLFNKWCWENWLAICRKLKLDPFLTPYTKINSRWIKDLNVKPKTIKTLEENLGITIQDIGVGKDFMSKTPKAMATKDKIDKWDLIKLKSFCTAKETTIRVNRQPTTWEKIFATYSSDKGLISRIYNELKQIYKKKTNNPIKKWAKDMNRHFSKEDIYAAKKHMKKCSSSLAIREMQIKTTMRYHLTPVRMAIIKKSGNNRCWRGCGEIGTLLHCWWDCKLVQPLWKSVWRFLRDLELEIPFDPAIPLLGIYPNEYKSCCYKDTCTRMFIAALFTIAKTWNQPKCPTMIDWIKKMWHIYTMEYYAAIKNDEFISFVGTWMKLETIILSKLSQEQKTKHRIFSLIGGN

SEQ ID NO: 60 - DNA, 3828 bp, synthetic construct
  misc_feature 1..3828: note = Description of Artificial Sequence: Synthetic polynucleotide
  source 1..3828: mol_type = other DNA
atgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaactaa

SEQ ID NO: 61 - protein, 1290 aa, synthetic construct
  REGION 1..1290: note = Description of Artificial Sequence: Synthetic polypeptide
MVIGTYISIITLNVNGLNAPTKRHRLAEWIQKQDPYICCLQETHFRPRDTYRLKVRGWKKIFHANGNQKKAGVAILISDKIDFKIKNVTRDKEGHYIMIQGSIQEEDITIINIYAPNIGAPQYIRQLLTAIKEEIDSNTIIVGDFNTSLTPMDRSSKMKINKETEALNDTIDQIDLIDIYRTFHPKTADYTFFSSAHGTFSRIDHILGHKSSLSKFKKIEIISSIFSDHNAMRLEMNHREKNVKKTNTWRLNNTLLNNQEITEEIKQEIKKYLETNDNENTTTQNLWDAAKAVLRGKFIAIQAYLKKQEKSQVNNLTLHLKKLEKEEQTKPKVSRRKEIIKIRAEINEIETKKTIAKINKTKSWFFEKINKIDKPLARLIKKKRERTQINKIRNEKGEVTTDTAEIQNILRDYYKQLYANKMDNLEEMDKFLERYNLPRLNQEETENINRPITSNEIETVIKNLPTNKSPGPDGFTGEFYQTFREELTPILLKLFQKIAEEGTLPNSFYEATITLIPKPDKDTTKKENYRPISLMNIDAKILNKILANRIQQHIKRIIHHDQVGFIPGMQGFFNIRKSINVIHHINKLKKKNHMIISIDAEKAFDKIQHPFMIKTLQKVGIEGTYLNIIKAIYDKPTANIILNGEKLKAFPLRSGTRQGCPLSPLLFNIVLEVLATAIREEKEIKGIQIGKEEVKLSLFADDMILYIENPKTATRKLLELINEYGKVAGYKINAQKSLAFLYTNDEKSEREIMETLPFTIATKRIKYLGINLPKETKDLYAENYKTLMKEIKDDTNRWRDIPCSWIGRINIVKMSILPKAIYRFNAIPIKLPMAFFTELEQIILKFVWRHKRPRIAKAVLRQKNGAGGIRLPDFRLYYKATVIKTIWYWHKNRNIDQWNKIESPEINPRTYGQLIYDKGGKDIQWRKDSLFNKWCWENWTATCKRMKLEYSLTPYTKINSKWIRDLNIRLDTIKLLEENIGRTLFDINHSKIFFDPPPRVMEIKTKINKWDLMKLQSFCTAKETINKTKRQPSEWEKIFANESTDKGLISKIYKQLIQLNIKETNTPIQKWAEDLNRHFSKEDIQTATKHMKRCSTSLIIREMQIKTTMRYHLTPVRMGIIRKSTNNKCWRGCGEKGTLLHCWWECKLIQPLWRTIWRFLKKLKIELPYDPAIPLLGIYPEKTVIQKDTCTRMFIAALFTIARSWKQPKCPSTDEWIKKMWYIYTMEYYSAIKRNEIGSFLETWMDLETVIQSEVSQKEKNKYRILTHICGTWKNGTDEPVCRTEIETQM

SEQ ID NO: 62 - DNA, 3873 bp, synthetic construct
  misc_feature 1..3873: note = Description of Artificial Sequence: Synthetic polynucleotide
  source 1..3873: mol_type = other DNA
atggtcataggaacatacatatcgataattaccttaaacgtgaatggattaaatgccccaaccaaaagacatagactggctgaatggatacaaaaacaagacccatatatatgctgtctacaagagacccacttcagacctagggacacatacagactgaaagtgaggggatggaaaaagatattccatgcaaatggaaatcaaaagaaagctggagtagctatactcatatcagataaaatagactttaaaataaagaatgttacaagagacaaggaaggacactacataatgatccagggatcaatccaagaagaagatataacaattataaatatatatgcacccaacataggagcacctcaatacataaggcaactgctaacagctataaaagaggaaatcgacagtaacacaataatagtgggggactttaacacctcacttacaccaatggacagatcatccaaaatgaaaataaataaggaaacagaagctttaaatgacacaatagaccagatagatttaattgatatatataggacattccatccaaaaacagcagattacacgttcttctcaagtgcgcacggaacattctccaggatagatcacatcttgggtcacaaatcaagcctcagtaaatttaagaaaattgaaatcatatcaagcatcttttctgaccacaacgctatgagattagaaatgaatcacagggaaaaaaacgtaaaaaagacaaacacatggaggctaaacaatacgttactaaataaccaagagatcactgaagaaatcaaacaggaaataaaaaaatacctagagacaaatgacaatgaaaacacgacgacccaaaacctatgggatgcagcaaaagcggttctaagagggaagtttatagctatacaagcctacctaaagaaacaagaaaaatctcaagtaaacaatctaaccttacacctaaagaaactagagaaagaagaacaaacaaaacccaaagttagcagaaggaaagaaatcataaagatcagagcagaaataaatgaaatagaaacaaagaaaacaatagcaaagatcaataaaactaaaagttggttctttgagaagataaacaaaattgataagccattagccagactcatcaagaaaaagagggagaggactcaaatcaataaaatcagaaatgaaaaaggagaagttacaacagacaccgcagaaatacaaaacatcctaagagactactacaagcaactttatgccaataaaatggacaacctggaagaaatggacaaattcttagaaaggtataaccttccaagactgaaccaggaagaaacagaaaatatcaacagaccaatcacaagtaatgaaattgaaactgtgattaaaaatcttccaacaaacaaaagtccaggaccagatggcttcacaggtgaattctatcaaacatttagagaagagctaacacccatccttctcaaactcttccaaaaaattgcagaagaaggaacactcccaaactcattctatgaggccaccatcaccctgataccaaaaccagacaaagacactacaaaaaaagaaaattacagaccaatatcactgatgaatatagatgcaaaaatcctcaacaaaatactagcaaacagaatccaacaacacattaaaaggatcatacaccacgatcaagtgggatttatcccagggatgcaaggattcttcaatatacgcaaatcaatcaatgtgatacaccatattaacaaattgaagaagaaaaaccatatgatcatctcaatagatgcagaaaaagcttttgacaaaattcaacacccatttatgataaaaactctccagaaagtgggcatagagggaacctacctcaacataataaaggccatatatgacaaacccacagcaaacatcattctcaatggtgaaaaactgaaagcatttcctctaagatcaggaacgagacaaggatgtccactctcaccactattattcaacatagttctggaagtcctagccacggcaatcagagaagaaaaagaaataaaaggaatacaaattggaaaagaagaagtaaaactgtcactgtttgcggatgacatgatactatacatagagaatcctaaaactgccaccagaaaactgctagagctaattaatgaatatggtaaagttgcaggttacaaaattaatgcacagaaatctcttgcattcctatacactaatgatgaaaaatctgaaagagaaattatggaaacactcccatttaccattgcaacaaaaagaataaaatacctaggaataaacctacctaaggagacaaaagacctgtatgcagaaaactataagacactgatgaaagaaattaaagatgataccaacagatggagagatataccatgttcttggattggaagaatcaacattgtgaaaatgagtatactacccaaagcaatctacagattcaatgcaatccctatcaaattaccaatggcattttttacggagctagaacaaatcatcttaaaatttgtatggagacacaaaagaccccgaatagccaaagcagtcttgaggcaaaaaaatggagctggaggaatcagactccctgacttcagactatactacaaagctacagtaatcaagacaatatggtactggcacaaaaacagaaacatagatcaatggaacaagatagaaagcccagagattaacccacgcacctatggtcaactaatctatgacaaaggaggcaaagatatacaatggagaaaagacagtctcttcaataagtggtgctgggaaaactggacagccacatgtaaaagaatgaaattagaatactccctaacaccatacacaaaaataaactcaaaatggattagagacctaaatataagactggacactataaaactcttagaggaaaacataggaagaacactctttgacataaatcacagcaagatctttttcgatccacctcctagagtaatggaaataaaaacaaaaataaacaagtgggacctaatgaaacttcaaagcttttgcacagcaaaggaaaccataaacaagacgaaaagacaaccctcagaatgggagaaaatatttgcaaatgaatcaacggacaaaggattaatctccaaaatatataaacagctcattcagctcaatatcaaagaaacaaacaccccaatccaaaaatgggcagaagacctaaatagacatttctccaaagaagacatacagacggccacgaagcacatgaaaagatgctcaacatcactaattattagagaaatgcaaatcaaaactacaatgaggtatcacctcactcctgttagaatgggcatcatcagaaaatctacaaacaacaaatgctggagagggtgtggagaaaagggaaccctcttgcactgttggtgggaatgtaaattgatacagccactatggagaacaatatggaggttccttaaaaaactaaaaatagaattaccatatgacccagcaatcccactactgggcatatacccagagaaaaccgtaattcaaaaagacacatgcacccgaatgttcattgcagcactatttacaatagccaggtcatggaagcaacctaaatgcccatcgacagacgaatggataaagaagatgtggtacatatatacaatggaatattactcagccataaaaaggaacgaaattgggtcatttttagagacgtggatggatctagagactgtcatacagagtgaagtaagtcagaaagagaaaaacaaatatcgtatattaacgcatatatgtggaacctggaaaaatggtacagatgaaccggtctgcaggacagaaattgagacacaaatgtaa

SEQ ID NO: 63 - protein, 9 aa, synthetic construct
  REGION 1..9: note = Description of Artificial Sequence: Synthetic peptide
PAAKRVKLD

SEQ ID NO: 64 - protein, 8 aa, synthetic construct
  REGION 1..8: note = Description of Artificial Sequence: Synthetic peptide
DYKDDDDK

SEQ ID NO: 65 - protein, 234 aa, synthetic construct
  REGION 1..234: note = Description of Artificial Sequence: Synthetic polypeptide
ASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYAMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 66 - protein, 9 aa, unidentified
  REGION 1..9: note = Description of Unknown: "LAGLIDADG" family peptide motif sequence
LAGLIDADG

SEQ ID NO: 67 - protein, 45 aa, synthetic construct
  REGION 1..45: note = Description of Artificial Sequence: Synthetic polypeptide
LYCRRLKIQVRKAAITSYEKSDGVYTGLSTRNQETYETLKHEKPP

SEQ ID NO: 68 - protein, 61 aa, synthetic construct
  REGION 1..61: note = Description of Artificial Sequence: Synthetic polypeptide
KVAKKPTNKAPHPKQEPQEINFPDDLPGSNTAAPVQETLHGCQPVTQEDGKESRISVQERQ

SEQ ID NO: 69 - protein, 415 aa, synthetic construct
  REGION 1..415: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQGSGSYEDMRGILYAAPQLRSIRGQPGPNHEEDADSYENM

SEQ ID NO: 70 - protein, 437 aa, synthetic construct
  REGION 1..437: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRLKIQVRKAAITSYEKSDGVYTGLSTRNQETYETLKHEKPPQKKVAKKPTNKAPHPKQEPQEINFPDDLPGSNTAAPVQETLHGCQPVTQEDGKESRISVQERQ

SEQ ID NO: 71 - protein, 438 aa, synthetic construct
  REGION 1..438: note = Description of Artificial Sequence: Synthetic polypeptide
EIQLVQSGGGLVKPGGSVRISCAASGYTFTNYGMNWVRQAPGKGLEWMGWINTHTGEPTYADSFKGRFTFSLDDSKNTAYLQINSLRAEDTAVYFCTRRGYDWYFDVWGQGTTVTVSSGGGGSGGGGSGGGGSDIQMTQSPSSLSASVGDRVTITCRASQDINSYLSWFQQKPGKAPKTLIYRANRLESGVPSRFSGSGSGTDYTLTISSLQYEDFGIYYCQQYDESPWTFGGGTKLEIKSGGGGSGALSNSIMYFSHFVPVFLPAKPTTTPAPRPPTPAPTIASQPLSLRPEACRPAAGGAVHTRGLDIYIWAPLAGTCGVLLLSLVITLYCRRLKIQVRKAAITSYEKSDGVYTGLSTRNQETYETLKHEKPPQKKVAKKPTNKAPHPKQEPQEINFPDDLPGSNTAAPVQETLHGCQPVTQEDGKESRISVQERQ

SEQ ID NO: 72 - protein, 44 aa, synthetic construct
  REGION 1..44: note = Description of Artificial Sequence: Synthetic polypeptide
MRNKKILKEDELLSETQQAAFHQIAMEPFEINVPKPKRRNGVNF

SEQ ID NO: 73 - protein, 50 aa, synthetic construct
  REGION 1..50: note = Description of Artificial Sequence: Synthetic polypeptide
MEQWDHFHNQQEDTDSCSESVKFDARSMTALLPPNPKNSPSLQEKLKSFK

SEQ ID NO: 74 - RNA, 203 nt, synthetic construct
  misc_feature 1..203: note = Description of Artificial Sequence: Synthetic polynucleotide
  misc_difference 66..185
  misc_feature 1..203: note = See specification as filed for detailed description of substitutions and preferred embodiments
  source 1..203: mol_type = other RNA
gctgggtttttccttgttcgcaccggacacctccagtgaccagacggcaaggtttttatcccagtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaaaaaaaaaaaaaaaaa

SEQ ID NO: 75 - protein, 33 aa, synthetic construct
  REGION 1..33: note = Description of Artificial Sequence: Synthetic polypeptide
GAAPAAAPAKQEAAAPAPAAKAEAPAAAPAAKA

SEQ ID NO: 76 - protein, 20 aa, synthetic construct
  REGION 1..20: note = Description of Artificial Sequence: Synthetic peptide
  SITE 1..20: note = This sequence may encompass 1-4 "Gly Gly Gly Gly Ser" repeating units
  REGION 1..20: note = See specification as filed for detailed description of substitutions and preferred embodiments
GGGGSGGGGSGGGGSGGGGS

SEQ ID NO: 77 - protein, 6 aa, synthetic construct
  REGION 1..6: note = Description of Artificial Sequence: Synthetic peptide
GGGGGG

SEQ ID NO: 78 - protein, 8 aa, synthetic construct
  REGION 1..8: note = Description of Artificial Sequence: Synthetic peptide
GGGGGGGG

SEQ ID NO: 79 - protein, 20 aa, synthetic construct
  REGION 1..20: note = Description of Artificial Sequence: Synthetic peptide
  SITE 1..20: note = This sequence may encompass 1-4 "Glu Ala Ala Ala Lys" repeating units
  REGION 1..20: note = See specification as filed for detailed description of substitutions and preferred embodiments
EAAAKEAAAKEAAAKEAAAK

SEQ ID NO: 80 - protein, 7 aa, Simian virus 40
PKKKRKV

SEQ ID NO: 81 - DNA, 21 bp, Simian virus 40
  source 1..21: mol_type = other DNA
ccaaagaagaagcggaaggtc

SEQ ID NO: 82 - protein, 11 aa, synthetic construct
  REGION 1..11: note = Description of Artificial Sequence: Synthetic peptide
PKKKRKVGGGS

SEQ ID NO: 83 - DNA, 33 bp, synthetic construct
  misc_feature 1..33: note = Description of Artificial Sequence: Synthetic oligonucleotide
  source 1..33: mol_type = other DNA
ccaaagaagaagcggaaggtcggcggcggcagc

SEQ ID NO: 84 - protein, 16 aa, unidentified
  REGION 1..16: note = Description of Unknown: Nucleoplasmin bipartite NLS sequence
KRPAATKKAGQAKKKK

SEQ ID NO: 85 - DNA, 48 bp, unidentified
  misc_feature 1..48: note = Description of Unknown: Nucleoplasmin bipartite NLS sequence
  source 1..48: mol_type = other DNA
aaaaggccggcggccacgaaaaaggccggccaggcaaaaaagaaaaag

SEQ ID NO: 86 - protein, 20 aa, synthetic construct
  REGION 1..20: note = Description of Artificial Sequence: Synthetic peptide
KRPAATKKAGQAKKKKGGGS

SEQ ID NO: 87 - DNA, 60 bp, synthetic construct
  misc_feature 1..60: note = Description of Artificial Sequence: Synthetic oligonucleotide
  source 1..60: mol_type = other DNA
aaaaggccggcggccacgaaaaaggccggccaggcaaaaaagaaaaagggcggcggcagc

SEQ ID NO: 88 - protein, 20 aa, synthetic construct
  REGION 1..20: note = Description of Artificial Sequence: Synthetic peptide
GGGSKRPAATKKAGQAKKKK

SEQ ID NO: 89 - DNA, 60 bp, synthetic construct
  misc_feature 1..60: note = Description of Artificial Sequence: Synthetic oligonucleotide
  source 1..60: mol_type = other DNA
ggcggcggcagcaaaaggccggcggccacgaaaaaggccggccaggcaaaaaagaaaaag

SEQ ID NO: 90 - protein, 11 aa, synthetic construct
  REGION 1..11: note = Description of Artificial Sequence: Synthetic peptide
GGGSPKKKRKV

SEQ ID NO: 91 - DNA, 33 bp, synthetic construct
  misc_feature 1..33: note = Description of Artificial Sequence: Synthetic oligonucleotide
  source 1..33: mol_type = other DNA
ggcggcggcagcccaaagaagaagcggaaggtc

SEQ ID NO: 92 - DNA, 12542 bp, synthetic construct
  misc_feature 1..12542: note = Description of Artificial Sequence: Synthetic polynucleotide
  source 1..12542: mol_type = other DNA
ctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgccaaagaagaagcggaaggtctgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaac

SEQ ID NO: 93 - DNA, 12554 bp, synthetic construct
  misc_feature 1..12554: note = Description of Artificial Sequence: Synthetic polynucleotide
  source 1..12554: mol_type = other DNA
cctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgggcggcggcagcccaaagaagaagcggaaggtctgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgat

SEQ ID NO: 94 - DNA, 12569 bp, synthetic construct
  misc_feature 1..12569: note = Description of Artificial Sequence: Synthetic polynucleotide
  source 1..12569: mol_type = other DNA
tcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgaaaaggccggcggccacgaaaaaggccggccaggcaaaaaagaaaaagtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaata

SEQ ID NO: 95 - DNA, 12581 bp, synthetic construct
  misc_feature 1..12581: note = Description of Artificial Sequence: Synthetic polynucleotide
  source 1..12581: mol_type = other DNA
tatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgggcggcggcagcaaaaggccggcggccacgaaaaaggccggccaggcaaaaaagaaaaagtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaa

SEQ ID NO: 96 - DNA, 12542 bp, synthetic construct
  misc_feature 1..12542: note = Description of Artificial Sequence: Synthetic polynucleotide
  source 1..12542: mol_type = other DNA
gtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgccaaagaagaagcggaaggtcggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctc

SEQ ID NO: 97 - DNA, 12554 bp, synthetic construct
  misc_feature 1..12554: note = Description of Artificial Sequence: Synthetic polynucleotide
  source 1..12554: mol_type = other DNA
ggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgccaaagaagaagcggaaggtcggcggcggcagcggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtc

SEQ ID NO: 98 - DNA, 12569 bp, synthetic construct
  misc_feature 1..12569: note = Description of Artificial Sequence: Synthetic polynucleotide
  source 1..12569: mol_type = other DNA
ggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgaaaaggccggcggccacgaaaaaggccggccaggcaaaaaagaaaaagggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtc

SEQ ID NO: 99 - DNA, 12581 bp, synthetic construct
  misc_feature 1..12581: note = Description of Artificial Sequence: Synthetic polynucleotide
  source 1..12581: mol_type = other DNA
caaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgaaaaggccggcggccacgaaaaaggccggccaggcaaaaaagaaaaagggcggcggcagcggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatctt

SEQ ID NO: 100 - DNA, 12542 bp, synthetic construct
  misc_feature 1..12542: note = Description of Artificial Sequence: Synthetic polynucleotide
  source 1..12542: mol_type = other DNA
ggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgccaaagaagaagcggaaggtcaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatc

SEQ ID NO: 101 - DNA, 12554 bp, synthetic construct
  misc_feature 1..12554: note = Description of Artificial Sequence: Synthetic polynucleotide
  source 1..12554: mol_type = other DNA
tggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgccaaagaagaagcggaaggtcggcggcggcagcaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctct

SEQ ID NO: 102 - DNA, 12569 bp, synthetic construct
  misc_feature 1..12569: note = Description of Artificial Sequence: Synthetic polynucleotide
  source 1..12569: mol_type = other DNA
gacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaaaaggccggcggccacgaaaaaggccggccaggcaaaaaagaaaaagaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctc

SEQ ID NO: 103 - DNA, 12581 bp, synthetic construct
  misc_feature 1..12581: note = Description of Artificial Sequence: Synthetic polynucleotide
  source 1..12581: mol_type = other DNA
agatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaaaaggccggcggccacgaaaaaggccggccaggcaaaaaagaaaaagggcggcggcagcaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccg

SEQ ID NO: 104 - DNA, 12542 bp, synthetic construct
  misc_feature 1..12542: note = Description of Artificial Sequence: Synthetic polynucleotide
  source 1..12542: mol_type = other DNA
agaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagccaaagaagaagcggaaggtctaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccg

SEQ ID NO: 105 - DNA, 12554 bp, synthetic construct
  misc_feature 1..12554: note = Description of Artificial Sequence: Synthetic polynucleotide
  source 1..12554: mol_type = other DNA
tcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagggcggcggcagcccaaagaagaagcggaaggtctaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtc

SEQ ID NO: 106 - DNA, 12569 bp, synthetic construct
  misc_feature 1..12569: note = Description of Artificial Sequence: Synthetic polynucleotide
  source 1..12569: mol_type = other DNA
accgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagaaaaggccggcggccacgaaaaaggccggccaggcaaaaaagaaaaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgt

SEQ ID NO: 107 - DNA, 12581 bp, synthetic construct
  misc_feature 1..12581: note = Description of Artificial Sequence: Synthetic polynucleotide
  source 1..12581: mol_type = other DNA
ccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagggcggcggcagcaaaaggccggcggccacgaaaaaggccggccaggcaaaaaagaaaaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgta

SEQ ID NO: 108 - DNA, 12602 bp, synthetic construct
  misc_feature 1..12602: note = Description of Artificial Sequence: Synthetic polynucleotide
  source 1..12602: mol_type = other DNA
tggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgtgagacagccgtcagaccatcaagactaggaagaaactgcatcaactaatgagcaaaatcaccagctaacatcatagtatacatgccaaagaagaagcggaaggtcggcggcggcagcaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagaaaaggccggcggccacgaaaaaggccggccaggcaaaaaagaaaaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctct

SEQ ID NO: 109 - DNA, 12541 bp, synthetic construct
  misc_feature 1..12541: note = Description of Artificial Sequence: Synthetic polynucleotide
  source 1..12541: mol_type = other DNA
tggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgggctccggcgagggcaggggaagccttctaacatgcggggacgtggaggaaaatcccggcccaggtagcggcccaaagaagaagcggaaggtcggcggcggcagcaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctct

SEQ ID NO: 110 - DNA, 12556 bp, synthetic construct
  misc_feature 1..12556: note = Description of Artificial Sequence: Synthetic polynucleotide
  source 1..12556: mol_type = other DNA
accgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcgtttaaactcgactctggctctatcgaatctccgtcgtttcgagcttacgcgaacagccgtggcgctcatttgctcgtcgggcatcgaatctcgtcagctatcgtcagcttacctttttggcagcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgctaatacgactcactatagggagaagtactgccaccatgggcaagaagcaaaatcgcaagacggggaattccaagacacaatccgctagcccaccacctaaagagcgttctagctcccctgctactgagcagtcctggatggaaaacgacttcgatgaactccgggaagagggatttaggcgatccaactattcagaactccgcgaagatatccagacaaaggggaaggaagtcgagaatttcgagaagaacctcgaggagtgcatcacccgtatcacaaacactgagaaatgtctcaaagaactcatggaacttaagacaaaagccagggagcttcgagaggagtgtcggagtctgagatccaggtgtgaccagctcgaggagcgcgtgagcgcgatggaagacgagatgaacgagatgaaaagagagggcaaattcagggagaagcgcattaagaggaacgaacagagtctgcaggagatttgggattacgtcaagaggcctaacctgcggttgatcggcgtccccgagagcgacgtagaaaacgggactaaactggagaatacacttcaagacatcattcaagaaaattttccaaacctggctcggcaagctaatgtgcaaatccaagagatccaacgcacaccccagcggtatagctctcggcgtgccacccctaggcatattatcgtgcgctttactaaggtggagatgaaagagaagatgctgcgagccgctcgggaaaagggaagggtgactttgaagggcaaacctattcggctgacggttgaccttagcgccgagacactccaggcacgccgggaatggggccccatctttaatatcctgaaggagaagaacttccagccacgaatctcttaccctgcaaagttgagttttatctccgagggtgagattaagtatttcatcgataaacagatgctgcgagacttcgtgacaactcgcccagctctcaaggaactgctcaaagaggctcttaatatggagcgcaataatagatatcaacccttgcagaaccacgcaaagatgggctccggcgagggcaggggaagccttctaacatgcggggacgtggaggaaaatcccggcccaggtagcggcaccggctctaactcacatatcaccatccttacacttaacattaacggcctcaactcagctatcaagcgccatcggctggccagctggatcaaatcacaggatccaagcgtttgttgcatccaagagacccacctgacctgtagagatactcaccgcctcaagatcaagggatggcgaaagatttatcaggcgaacggtaagcagaagaaagccggagtcgcaattctggtctcagacaagacggatttcaagcccaccaaaattaagcgtgataaggaaggtcactatattatggtgaaaggcagcatacagcaggaagaacttaccatattgaacatctacgcgccaaacaccggcgcacctcgctttatcaaacaggtcctgtccgatctgcagcgagatctggattctcatacgttgattatgggtgatttcaatacaccattgagcaccctggatcgcagcaccaggcaaaaggtaaataaagacacgcaagagctcaatagcgcactgcatcaggcagatctcattgatatttatcgcactcttcatcctaagagtaccgagtacacattcttcagcgccccacatcatacatactcaaagatcgatcatatcgtcggctcaaaggctctgctgtcaaagtgcaagcgcacagagataattacaaattacctgtcagatcatagcgcgatcaagctcgagctgagaatcaagaacctgacccagagccggagtaccacttggaagcttaataacctgctgctcaacgattattgggtccacaatgagatgaaggcagagattaaaatgttcttcgaaacaaatgagaataaggatactacctatcaaaacctttgggatgcctttaaggccgtctgcagaggcaagttcatcgccctcaacgcctataaaagaaaacaagagagatctaagatcgatactctcacctctcagctgaaggagttggagaaacaggaacagacccactccaaggcgtcaagacggcaggagatcacaaagattcgcgccgagttgaaagagatcgaaacccaaaagactcttcagaaaattaacgagtctcgtagttggttcttcgagcggattaataagatagacagacctctggcacgactgattaagaagaagcgcgaaaagaaccagattgataccatcaagaacgacaagggcgacatcactactgacccgaccgagatccagaccactattcgggagtattataagcatttgtatgctaacaagcttgagaacctggaagagatggacacttttctggatacctatactctgccacggcttaatcaagaggaagtcgagtccctcaaccgcccaattacaggaagcgagattgtggccataattaactccctgccgacaaagaaatctcctggtccggacgggtttacagctgagttttatcaacggtatatggaagagcttgtaccgtttctgctcaagctctttcagtctatagaaaaggaaggcatcttgcccaattccttctacgaagcttctataatacttattcccaaaccaggacgcgataccacaaagaaggaaaacttccggcccattagtctcatgaatatcgacgctaaaatattgaacaagattctcgccaacagaatccaacaacatattaagaaattgatacatcacgaccaggtggggtttatacctggcatgcagggctggtttaacatccggaagagtattaacgtcattcaacacattaatagagctaaggataagaatcatatgatcatctctatagacgcggaaaaggcattcgataagattcagcagccatttatgctcaagactctgaacaaactcggcatcgacggaacatattttaagattattcgcgcaatttacgataagccgactgctaacattatccttaacggccaaaagctcgaggcctttccgctcaagactggaacccgccaaggctgtcccctctccccgcttttgtttaatattgtactcgaggtgctggctagggctattcgtcaagagaaagagattaaagggatacagctcgggaaggaagaggtcaagctttccttgttcgccgatgatatgattgtgtacctggagaatcctattgtgtctgctcagaaccttcttaaacttatttctaactttagcaaggtcagcggctataagattaacgtccagaaatctcaggcctttctgtacacaaataatcgacagaccgaatcccagataatgggtgagcttccgtttgtcatagccagcaaaaggataaagtatctcggaatccagctgacacgagacgttaaagatttgtttaaggaaaattacaagcctctcctgaaagagattaaggaagatactaataagtggaagaatatcccctgttcatgggttggcagaatcaacatagtgaagatggcaatacttcctaaagtgatatatcgctttaacgccatcccaattaaactgcctatgaccttctttacggagctcgagaaaacaacccttaaatttatatggaatcaaaagagagcaagaatagcgaagtccatcttgagccagaagaataaggccggtgggattactttgcctgattttaagttgtattataaagccacagtaactaagacagcctggtattggtatcagaatagagacatcgaccagtggaatcggaccgaaccatcagagataatgccccacatctataattaccttatattcgataagccagaaaagaataaacagtggggcaaagacagcctcttcaacaagtggtgttgggagaattggctggccatatgccggaaactcaagctcgacccctttcttacaccctacactaaaatcaacagtaggtggatcaaggacttgaatgtcaagccaaagactataaagacactggaagagaatcttgggatcacaatacaagatataggcgtcggcaaagattttatgtcaaagacgcccaaggccatggccactaaggataagattgataagtgggaccttattaagctcaaaagcttctgtactgccaaggagaccacgatcagagttaataggcagcccactacatgggaaaagattttcgccacttattcatcagataaggggttgataagcagaatatataacgagctgaagcagatctacaagaagaaaacgaataatcccatcaagaagtgggcaaaagatatgaacaggcattttagcaaagaggatatctacgccgcgaagaagcatatgaagaagtgtagttcaagcttggccattcgtgagatgcagattaagacgaccatgcgataccaccttaccccagtgaggatggcaattatcaagaaatctggcaataatagatgttggcggggctgtggcgagattggcaccctgctccattgctggtgggattgcaagctggtgcagccgctttggaaatcagtctggcgctttctgagggacctcgagcttgagattcccttcgatcccgcaattcccttgctcggaatctatcctaacgaatacaagagctgttgttacaaggatacgtgtacccggatgttcatcgcggccttgtttacgatagctaagacgtggaatcagcctaagtgccccacaatgatcgattggatcaagaaaatgtggcatatttataccatggagtattacgcagcaattaagaatgacgaatttatttccttcgttgggacctggatgaagctggagactattattctgagcaagctgtctcaggagcaaaagacaaagcatagaatcttctctctcattggtggtaacgactacaaagacgatgacgacaagaaaaggccggcggccacgaaaaaggccggccaggcaaaaaagaaaaagtaaagcgcttctagaagttgtctcctcctgcactgactgactgatacaatcgatttctggatccgcaggcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgctgagagacacaaaaaattccaacacactattgcaatgaaaataaatttcctttattagccagaagtcagatgctcaaggggcttcatgatgtccccataatttttggcagagggaaaaagatctcagtggtatttgtgagccagggcattggccttctgataggcagcctgcacctgaggagtgcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgggcctgctgccggctctgcggcctcttccgcgtcttcgccttcgccctcagacgagtcggatctccctttgggccgcctccccgcctgtctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaagcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaggaggtagccaacatgattgaacaagatggattgcacgcaggttctcccgccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccacggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctagtatgtaagccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgt

SEQ ID NO: 111 - RNA, 48 nt, synthetic construct
  misc_feature 1..48: note = Description of Artificial Sequence: Synthetic oligonucleotide
  source 1..48: mol_type = other RNA
ctaggaatctggaagtaccgaggaaactcggtacttcctgtgtcctag

SEQ ID NO: 112 - RNA, 37 nt, synthetic construct
  misc_feature 1..37: note = Description of Artificial Sequence: Synthetic oligonucleotide
  source 1..37: mol_type = other RNA
atatggaagatcctggggaactgggatcttcctaagt

SEQ ID NO: 113 - RNA, 76 nt, synthetic construct
  source 1..76: mol_type = other RNA
gcgcatttagctcagnngggagagcgccagactgaananctggagctcctgtgtncgatccacagaattcgcacca